(19) United States
(12) Patent Application Publication
(10) Pub. No.: US 2011/0053897 A1
(43) Pub. Date: Mar. 3, 2011

(54) COMPOUNDS AND COMPOSITIONS AS SYK KINASE INHIBITORS

(75) Inventors: Jianwei CHE, San Diego, CA (US); Bei Chen, San Diego, CA (US); Qiang Ding, San Diego, CA (US); Xueshi Hao, San Diego, CA (US); Xiaohui He, San Diego, CA (US); Songchun Jiang, San Diego, CA (US); Qihui Jin, San Diego, CA (US); Yunho Jin, San Diego, CA (US); Hong Liu, San Diego, CA (US); Yahua Liu, San Diego, CA (US); Barun Okram, San Diego, CA (US); Tetsuo Uno, San Diego, CA (US); Xu Wu, San Diego, CA (US); Kunyong Yang, San Diego, CA (US); Xuefeng Zhu, San Diego, CA (US)

(73) Assignee: IRM LLC, Hamilton (BM)

(21) Appl. No.: 12/843,771

(22) Filed: Jul. 26, 2010

Related U.S. Application Data
(60) Provisional application No. 61/229,975, filed on Jul. 30, 2009.

(51) Int. Cl. A61K 31/635; C07D 471/04; A61K 31/497; A61K 31/5377; A61K 31/506; A61K 31/444; A61P 11/06; A61P 19/02; A61P 35/00; A61P 7/06; A61P 25/00

(52) U.S. Cl. 514/156; 544/405; 544/122; 544/331; 546/122; 544/328; 544/127; 544/231; 540/575; 514/255.05; 514/234.5; 514/275; 514/300; 514/256; 514/210.21; 514/210.16

(57) ABSTRACT

Provided herein area novel class of compounds, pharmaceutical compositions comprising such compounds and methods of using such compounds to treat or prevent diseases or disorders associated with abnormal or deregulated Syk kinase activity.

### CROSS-REFERENCE TO RELATED APPLICATIONS

This application claims the benefit of U.S. Provisional Patent Application No. 61/229,975, filed Jul. 30, 2009, the disclosure of which is incorporated herein by reference in its entirety and for all purposes.

### FIELD OF THE INVENTION

The invention relates to protein kinase inhibitors, and methods of using such compounds.

### BACKGROUND OF THE INVENTION

Protein kinases (PK) are a large set of structurally related phosphoryl transferases having highly conserved structures and catalytic functions. Protein kinases are enzymatic components of the signal transduction pathways which catalyze the transfer of the terminal phosphate from ATP to the hydroxy group of tyrosine, serine and/or threonine residues of proteins, and are therefore categorized into families by the substrates they phosphorylate: Protein Tyrosine Kinases (PTK), and Protein Serine/Threonine Kinases.

Protein kinases play a critical role in the control of cell growth and differentiation and are responsible for the control of a wide variety of cellular signal transduction processes, wherein protein kinases are key mediators of cellular signals leading to the production of growth factors and cytokines. The overexpression or inappropriate expression of normal or mutant protein kinases plays a significant role in the development of many diseases and disorders including, central nervous system disorders such as Alzheimer's, inflammatory disorders such as arthritis, bone diseases such as osteoporosis, metabolic disorders such as diabetes, blood vessel proliferative disorders such as angiogenesis, autoimmune diseases such as rheumatoid arthritis, ocular diseases, cardiovascular disease, atherosclerosis, cancer, thrombosis, psoriasis, restenosis, schizophrenia, pain sensation, transplant rejection and infectious diseases such as viral, and fungal infections.

Examples of protein-tyrosine kinases include, but are not limited to, Irk, IGFR-1, Zap-70, Bmx, Btk, CHK (Csk homologous kinase), CSK (C-terminal Src Kinase), Itk-1, Src (c-Src, Lyn, Fyn, Lck, Hck, Yes, Blk, Fgr and Frk), Syk, Tec, Txk/Rlk, Abl, EGFR (EGFR-1/ErbB-1, ErbB-2/NEU/HER-2, ErbB-3 and ErbB-4), FAK, FGF1R (also FGFR1 or FGR-1), FGF2R (also FGR-2), MET (also Met-I or c-MET), PDGFR (.alpha. and .beta.), Tie-1, Tie-2 (also Tek-1 or Tek), VEGFR1 (also FLT-1), VEGFR2 (also KDR), FLT-3, FLT-4, c-KIT, JAK1, JAK2, JAK3, TYK2, LOK, RET, TRKA, PYK2, ALK (Anaplastic Lymphoma Kinase), EPHA (1-8), EPHB (1-6), RON, Fes, Fer or EPHB4 (also EPHB4-1).

Examples of protein-serine/threonine kinases include, but are not limited to, Ark, ATM (1-3), CamK (1-IV), CamKK, Chk1 and 2 (Checkpoint kinases), CK1, CK2, Erk, IKK-I (also IKK-ALPHA or CHUK), IKK-2 (also IKK-BETA), Ilk, Jnk (1-3), LimK (1 and 2), MLK3Raf (A, B, and C), CDK (1-10), PKC (including all PKC subtypes), Plk (1-3), NIK, Pak (1-3), PDK1, PKR, RhoK, RIP, RIP-2, GSK3 (.alpha. and .beta.), PKA, P38, Erk (1-3), PKB (including all PKB subtypes) (also AKT-1, AKT-2, AKT-3 or AKT3-1), IRAK1, FRK, SGK, TAK1 or Tp1-2 (also COT).

### SUMMARY OF THE INVENTION

Provided herein are compounds and pharmaceutical compositions thereof, which are useful as Syk kinase inhibitors.

In one aspect provided herein are compounds having the structure of Formula (I), and the pharmaceutically acceptable salts, pharmaceutically acceptable solvates (e.g. hydrates), the N-oxide derivatives, individual stereoisomers and mixture of stereoisomers thereof:

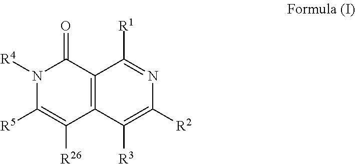

wherein:
R¹ is —NR⁶R⁷, a 4-8 membered heterocycloalkyl containing 1 to 2 heteroatoms independently selected from N, O and S or a 4-8 membered heterocycloalkyl containing 1 to 2 heteroatoms independently selected from N, O and S which is substituted with 1 to 3 substituents independently selected from hydroxyl and hydroxyl-C₁-C₆alkyl;
R² is selected from —NR⁸R¹⁰, R¹⁵, —C(O)R¹², —(CR¹²R¹²)ₙR¹⁴, —CR¹²═NOR¹², C₁-C₆alkyl, C₂-C₆alkene, phenyl, C₁₀aryl, C₁₄aryl, a 5, 6, 9, 10 or 14 membered heteroaryl containing 1 to 3 heteroatoms independently selected from N, O and S, and a 4-8 membered heterocycloalkyl containing 1 to 2 heteroatoms independently selected from N, O and S,
or R² is selected from C₁-C₆alkyl, C₂-C₆alkene, phenyl, C₁₀aryl, C₁₄aryl, a 5, 6, 9, 10 or 14 membered heteroaryl containing 1 to 3 heteroatoms independently selected from N, O and S, and a 4-8 membered heterocycloalkyl containing 1 to 2 heteroatoms independently selected from N, O and S, each of which is substituted with 1 to 3 substituents independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl;
R⁴ is H, C₁-C₆alkyl, deuterated C₁-C₆alkyl, —CD₃, C₁-C₆haloalkyl, C₂-C₆alkene, hydroxyl-C₁-C₆alkyl, R¹⁵, —(CR²⁷R²⁷)₁₋₆R¹⁴, —(CR²⁷R²⁷)(CR²⁷R²⁵)R¹¹, —(CR²⁷R²⁷)(CR²⁷R²⁵)R²⁵, —C(R²⁷R²⁵R²⁵) or —(CR²⁷R²⁷)ₙR¹¹;
   each R³ and each R⁵ are independently selected from H, halogen and C₁-C₆alkyl;
R⁶ is H, phenyl, C₁₀aryl, C₁₄aryl, C₁-C₆alkyl, C₃-C₈cycloalkyl, R¹⁵, —S(O)₂R¹³, —(CR¹²R¹²)₁₋₆R¹⁰, a 5, 6, 9, 10 or 14 membered heteroaryl containing 1 to 3 heteroatoms independently selected from N, O and S, or a 4-8 membered heterocycloalkyl containing 1 to 2 heteroatoms independently selected from N, O and S,
or R⁶ is phenyl, C₁₀aryl, C₁₄aryl, C₁-C₆alkyl, C₃-C₈cycloalkyl, a 5, 6, 9, 10 or 14 membered heteroaryl containing 1 to 3 heteroatoms independently selected from N, O and S, or a 4-8 membered heterocycloalkyl containing 1 to 2 heteroatoms independently selected from N, O and S, each of which is substituted with 1 to 3 substituents independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —C(O)R¹², —C(O)R¹³, —C(O)R¹⁵, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)₁₋₆R¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —C(R¹²R¹²R¹⁴), —(CR¹²R¹²)ₙR¹¹, —C(O)(CR¹²R¹²)₁₋₆R¹⁴, —C(O)C(R¹²R¹²R¹⁴), —NR¹²R¹², —S(O)₂NR¹²(CR¹²R¹²)₁₋₆R¹⁴, —S(O)₂NR¹²R¹², —S(O)₂R¹², C(O)C(R¹²R¹⁴)(C(R¹²R¹²))ₙR¹⁴, —(CR¹²R¹²)ₙC(O)OR¹², —C(O)N(R¹²)(CR¹²R¹²)ₙR¹¹, —(CR¹²R¹²)ₙC(O)R¹⁴, —(CR¹²R¹²)ₙC(R¹²R¹⁴)(C(R¹²R¹²))ₙR¹⁴ and —(CR¹²R¹²)ₙC(O)NR¹²(CR¹²R¹²)₁₋₆R¹⁴;
R⁷ is H or C₁-C₆ alkyl;
R⁸ is H or C₁-C₆ alkyl;
R¹⁰ is phenyl, C₁₀aryl, C₁₄aryl, a 5, 6, 9, 10 or 14 membered heteroaryl containing 1 to 3 heteroatoms independently selected from N, O and S, an N-oxide of a 5-6 membered heteroaryl containing 1-3 N heteroatoms, a 4-8 membered heterocycloalkyl containing 1 to 2 heteroatoms independently selected from N, O and S, C₃-C₈cycloalkyl or —(CR¹²R¹²)ₙR¹¹,
or R¹⁰ is phenyl, C₁₀aryl, C₁₄aryl, a 5, 6, 9, 10 or 14 membered heteroaryl containing 1 to 3 heteroatoms independently selected from N, O and S, an N-oxide of a 5-6 membered heteroaryl containing 1-3 N heteroatoms, a 4-8 membered heterocycloalkyl containing 1 to 2 heteroatoms independently selected from N, O and S, or C₃-C₈cycloalkyl, each of which is substituted with 1 to 3 substituents independently selected from halogen, hydroxyl, —NO₂, —CN, —C₁-C₆alkyl, —C₂-C₆alkene, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, R¹⁵, R¹¹, —OR¹², —OR¹¹, —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴, —O(CR¹²R¹²)₁₋₆R¹⁴, —O(CR¹²R¹²)ₙR¹¹, —(CR³R³)₁₋₆R¹⁴, —(CR¹²R¹²)ₙC(O)NR¹²(CR¹²R¹²)₁₋₆R¹⁴, —C(O)NR²⁷(CR¹²R¹²)ₙR¹¹, —(CR¹²R¹²)ₙC(O)NR¹²OR¹², —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₆R¹⁴, —C(O)(CR¹²R¹²)₁₋₆R¹⁴, and —C(O)C(R¹²R¹²R¹⁴);
R¹¹ is phenyl, C₁₀aryl, C₁₄aryl, a 5, 6, 9, 10 or 14 membered heteroaryl containing 1 to 4 heteroatoms independently selected from N, O and S, C₃-C₈cycloalkyl, or a 4-8 membered heterocycloalkyl containing 1 to 2 heteroatoms independently selected from N, O and S,
or R¹¹ is phenyl, C₁₀aryl, C₁₄aryl, a 5, 6, 9, 10 or 14 membered heteroaryl containing 1 to 4 heteroatoms independently selected from N, O and S, C₃-C₈cycloalkyl, or a 4-8 membered heterocycloalkyl containing 1 to 2 heteroatoms independently selected from N, O and S, each of which is substituted with 1 to 3 substituents independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴;
each R¹² is independently selected from H, C₁-C₆alkyl, hydroxyl-C₁-C₆alkyl and C₃-C₈cycloalkyl, or each R¹² is independently a C₁-C₆alkyl that together with N they are attached form a heterocycloalkyl;
R¹³ is H, C₁-C₆alkyl, halo-substituted C₁-C₆alkyl or a 4-8 membered heterocycloalkyl containing 1 to 2 heteroatoms independently selected from N, O and S;
R¹⁴ is H, halogen, hydroxyl, hydroxyl-C₁-C₆alkyl, R¹³, —OR¹³, —OR¹², —O(CR¹²R¹²)ₙOR¹³, C(O)R¹³, —N(R¹²)₂, —NR¹²OR¹³, —CN, —C(O)N(R¹²)₂, —S(O)₂R¹³, —(CR¹²R¹²)ₙOR¹³, C(O)R¹⁰, —OC(O)R¹³, —C(O)OR¹³, —S(O)₂N(R¹²)₂, —N(R¹²R¹⁰), —N(R¹²R¹¹), —(CR¹²R¹²)ₙN(R¹²)₂, —NR¹²C(O)(R¹²), —(CR¹²R¹²)ₙR¹³, —N(R¹²)C(O)(CR¹²R¹²)ₙOR¹³, —N(R¹²)(CR¹²R¹²)ₙOR¹³, —N(R¹²)(CR¹²R¹²)ₙR¹⁰, —C(O)N(R¹²)₂, —N(R¹²)C(O)R¹³, —N(R¹²)C(O)OR¹³, —(CR¹²R¹²)ₙR¹⁰, and R¹⁵;
R¹⁵ is

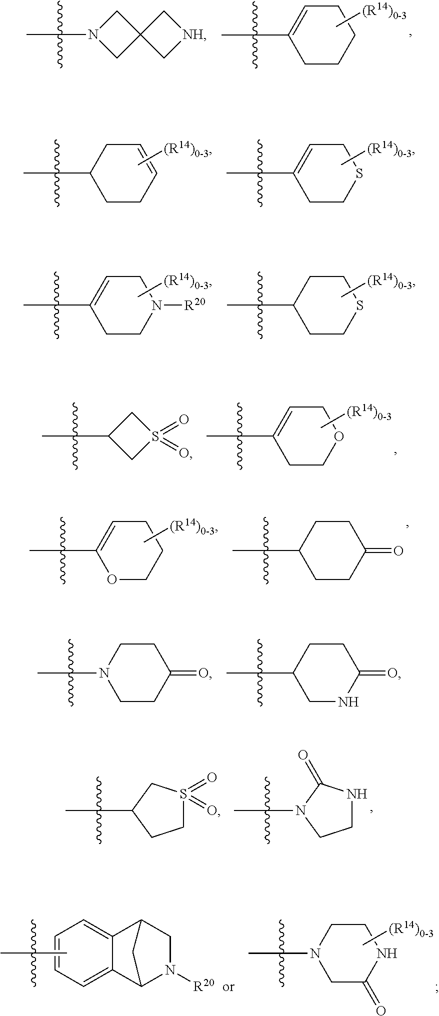

R²⁰ is H, —C₁-C₆alkyl, hydroxyl-C₁-C₆alkyl, —(CR¹²R¹²)₁₋₆R¹⁴ or —(CR¹²R¹²)ₙC(O)R¹³;
each R²⁵ is independently selected from H, hydroxyl, —C₁-C₆alkyl, —C₁-C₆haloalkyl, hydroxyl-C₁-C₆alkyl and —(CR¹²R¹²)ₙR¹⁴;
R²⁶ is H, halogen or C₁-C₆ alkyl;
each R²⁷ is independently selected from H or C₁-C₆alkyl, and
each n is independently 0, 1, 2, 3, 4, 5 or 6.

In certain embodiments of such compounds of Formula (I), R¹ is —NR⁶R⁷. In other embodiments of such compounds of Formulas (I), R⁷ and R⁸ are H.

In certain embodiments of the aforementioned compounds of Formula (I), R⁶ is H, phenyl, C₁-C₆alkyl, C₃-C₈cycloalkyl, R¹⁵, —S(O)₂R¹³, —(CR¹²R¹²)₁₋₆R¹⁰, a 5, 9, 10 or 14 membered heteroaryl containing 1 to 3 heteroatoms independently selected from N, O and S, or a 4-8 membered heterocycloalkyl containing 1 to 2 heteroatoms independently selected from N, O and S, and each n is independently 0, 1, 2, 3 or 4, while in other embodiments of the aforementioned compounds of Formula (I), R⁶ is phenyl, C₁-C₆alkyl, C₃-C₈cycloalkyl, a 5, 6, 9, 10 or 14 membered heteroaryl containing 1 to 3 heteroatoms independently selected from N, O and S, or a 4-8 membered heterocycloalkyl containing 1 to 2 heteroatoms independently selected from N, O and S, each of which is substituted with 1 to 3 substituents independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —C(O)R¹², —C(O)R¹³, —C(O)R¹⁵, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)₁₋₆R¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —C(R¹²R¹²R¹⁴), —(CR¹²R¹²)ₙR¹¹, —C(O)(CR¹²R¹²)₁₋₆R¹⁴, —C(O)C(R¹²R¹²R¹⁴), —NR¹²R¹², —S(O)₂NR¹²(CR¹²R¹²)₁₋₆R¹⁴, —S(O)₂NR¹²R¹², —S(O)₂R¹², —(CR¹²R¹²)ₙC(O)OR¹², C(O)C(R¹²R¹⁴)(C(R¹²R¹²))ₙR¹⁴, —C(O)N(R¹²)(CR¹²R¹²)ₙR¹¹, —(CR¹²R¹²)ₙC(O)R¹⁴, —(CR¹²R¹²)ₙC(R¹²R¹⁴)(C(R¹²R¹²))ₙR¹⁴ and —(CR¹²R¹²)ₙC(O)NR¹²(CR¹²R¹²)₁₋₆R¹⁴, and each n is independently 0, 1, 2, 3 or 4.

In certain embodiments of the aforementioned compounds of Formula (I), R⁶ is H, phenyl, C₁-C₆alkyl, C₃-C₈cycloalkyl, R¹⁵, —S(O)₂R¹³, —(CR¹²R¹²)₁₋₆R¹⁰, a 5, 6 or 9 membered heteroaryl containing 1 to 3 heteroatoms independently selected from N, O and S, or a 4-8 membered heterocycloalkyl containing 1 to 2 heteroatoms independently selected from N, O and S, and each n is independently 0, 1, 2, 3 or 4, while in other embodiments R⁶ is phenyl, C₁-C₆alkyl, C₃-C₈cycloalkyl, a 5, 6 or 9 membered heteroaryl containing 1 to 3 heteroatoms independently selected from N, O and S, or a 4-8 membered heterocycloalkyl containing 1 to 2 heteroatoms independently selected from N, O and S, each of which is optionally substituted with 1 to 3 substituents independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —(CR¹²R¹²)₁₋₆R¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)ₙR¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —(CR¹²R¹²)ₙR¹¹, —NR¹²R¹², —S(O)₂NR¹²(CR¹²R¹²)₁₋₆R¹⁴, —S(O)₂NR¹²R¹², —S(O)₂R¹², —(CR¹²R¹²)ₙC(O)NR¹²(CR¹²R¹²)₁₋₆R¹⁴, and —C(O)N(R¹²)(CR¹²R¹²)ₙR¹¹, and each n is independently 0, 1, 2, 3 or 4.

In certain embodiments of the aforementioned compounds of Formula (I), R⁶ is

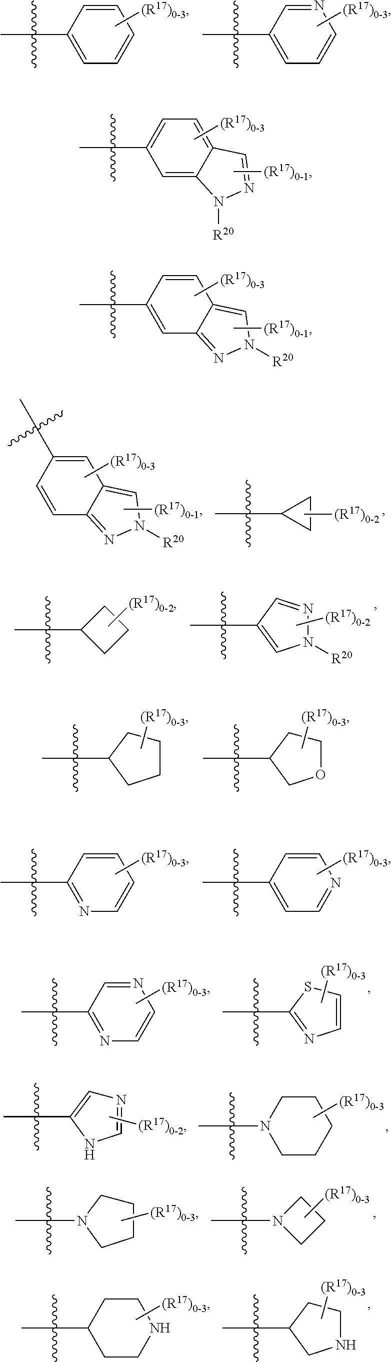

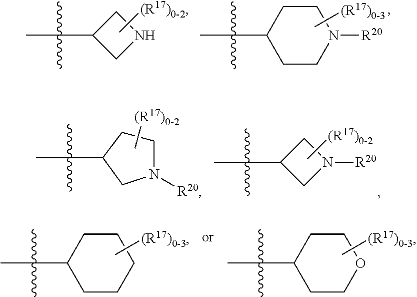

wherein
each R¹⁷ is independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)₁₋₆R¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —(CR¹²R¹²)ₙR¹¹, —NR¹²R¹², —S(O)₂NR¹²(CR¹²R¹²)₁₋₆R¹⁴, —S(O)₂NR¹²R¹², —S(O)₂R¹², —(CR¹²R¹²)ₙC(O)NR¹²(CR¹²R¹²)₁₋₆R¹⁴, and —C(O)N(R¹²)(CR¹²R¹²)ₙR¹¹;
R²⁰ is H, —C₁-C₆alkyl, hydroxyl-C₁-C₆alkyl or —(CR¹²R¹²)ₙR¹⁰, and
each n is independently 0, 1, 2, 3 or 4.

In certain embodiments of the aforementioned compounds of Formula (I), R⁶ is C₁-C₆alkyl or C₁-C₆alkyl substituted with 1 to 3 substituents independently selected from halogen, hydroxyl, C₁-C₆alkyl, C₁-C₆haloalkyl, hydroxyl-C₁-C₆alkyl, —OR¹², —O(CR¹²R¹²)ₙOR¹³, —C(O)R¹³, —N(R¹²)₂, —NR¹²OR¹³, —CN, —C(O)N(R¹²)₂, —S(O)₂R¹³ and R¹³.

In certain embodiments of the aforementioned compounds of Formula (I), R¹ is a 4-8 membered heterocycloalkyl containing 1 to 2 heteroatoms independently selected from N, O and S, or a 4-8 membered heterocycloalkyl containing 1 to 2 heteroatoms independently selected from N, O and S substituted with 1 to 3 substituents independently selected from hydroxyl and hydroxyl-C₁-C₆alkyl.

In certain embodiments of the aforementioned compounds of Formula (I), R¹ is selected from

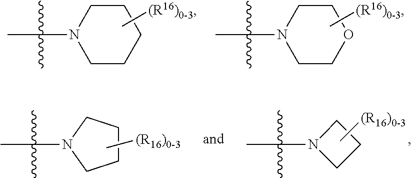

wherein each R¹⁶ is independently selected from hydroxyl and hydroxyl-C₁-C₆alkyl.

In certain embodiments of the aforementioned compounds of Formula (I), R² is R¹⁵, —C(O)R¹², —(CR¹²R¹²)ₙR¹⁴, —CR¹²═NOR¹², C₁-C₆alkyl, C₂-C₆alkene, a C₁-C₆alkyl substituted with 1 to 3 substituents independently selected from —OR¹², —C(O)OR¹², —C(O)R¹⁰, —N(R¹²)₂, —(CR¹²R¹²)ₙR¹⁴, C₁-C₆alkyl and hydroxyl-C₁-C₆alkyl or a C₂-C₆alkene substituted with 1 to 3 substituents independently selected from —OR¹², —C(O)OR¹², —C(O)R¹⁰, —N(R¹²)₂, —(CR¹²R¹²)ₙR¹⁴, C₁-C₆alkyl and hydroxyl-C₁-C₆alkyl.

In certain embodiments of the aforementioned compounds of Formula (I), R² is selected from phenyl, a 5, 6, or 9 membered heteroaryl containing 1 to 3 N heteroatoms, and a 4-8 membered heterocycloalkyl containing 1 to 2 heteroatoms independently selected from N, O and S and each n is independently 0, 1, 2, 3 or 4, while in other embodiments R² is selected from phenyl, a 5, 6, or 9 membered heteroaryl containing 1 to 3 N heteroatoms, and a 4-8 membered heterocycloalkyl containing 1 to 2 heteroatoms independently selected from N, O and S each of which is substituted with 1 to 3 substituents independently selected from —OR¹², —C(O)OR¹², —C(O)R¹⁰, —N(R¹²)₂, —(CR¹²R¹²)ₙR¹⁴, C₁-C₆alkyl and hydroxyl-C₁-C₆alkyl and each n is independently 0, 1, 2, 3 or 4.

In certain embodiments of the aforementioned compounds of Formula (I), R² is selected from

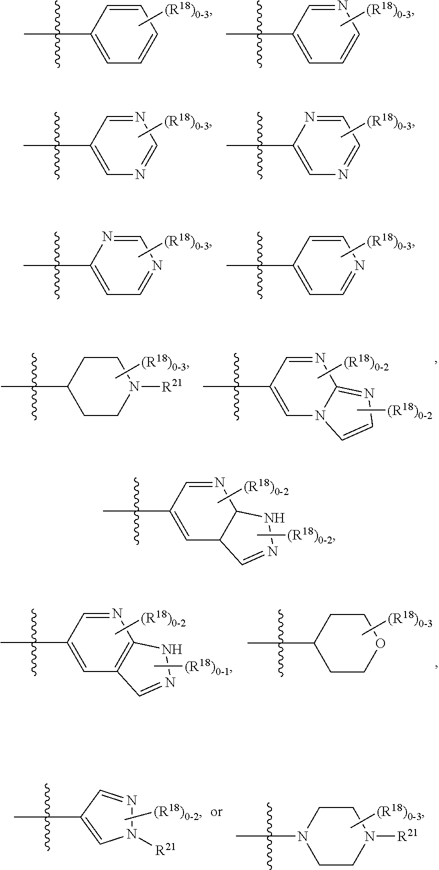

wherein each R¹⁸ is independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl; each R¹² is independently selected from H, —C₁-C₆alkyl and C₃-C₈cycloalkyl; R¹⁴ is —OR¹², R²¹ is H, C₁-C₆alkyl, —(CR¹²R¹²)₁₋₄R¹⁴ or hydroxyl-C₁-C₆alkyl, and each n is independently 0, 1, 2, 3 or 4.

In certain embodiments of the aforementioned compounds of Formula (I), R² is selected from,

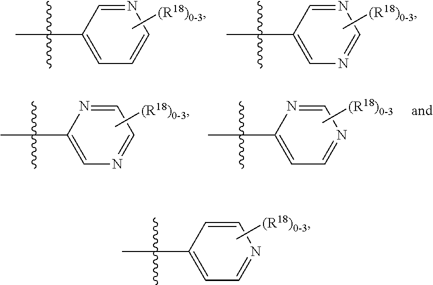

wherein each R¹⁸ is independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl and each n is independently 0, 1, 2, 3 or 4.

In certain embodiments of the aforementioned compounds of Formula (I), R² is —NR⁸R¹⁰.

In certain embodiments of the aforementioned compounds of Formula (I), R¹⁰ is phenyl, a 5, 6 or 9 membered heteroaryl containing 1 to 3 N heteroatoms, an N-oxide of a 5-6 membered heteroaryl containing 1-3 N heteroatoms, a 4-8 membered heterocycloalkyl containing 1 to 2 heteroatoms independently selected from N, O and S, C₃-C₈cycloalkyl or —(CR¹²R¹²)ₙR¹¹, while in other embodiments R¹⁰ is phenyl, a 5, 6 or 9 membered heteroaryl containing 1 to 3 N heteroatoms, an N-oxide of a 5-6 membered heteroaryl containing 1-3 N heteroatoms, a 4-8 membered heterocycloalkyl containing 1 to 2 heteroatoms independently selected from N, O and S, C₃-C₈cycloalkyl each of which is substituted with 1 to 3 substituents independently selected from halogen, hydroxyl, —NO₂, —CN, —C₁-C₆alkyl, —C₂-C₆alkene, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, R¹⁵, R¹¹, —OR¹², —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴, —(CR¹²R¹²)ₙC(O)NR¹²OR¹², —O(CR¹²R¹²)ₙR¹⁴, —O(CR¹²R¹²)ₙR¹¹, —(CR³R³)₁₋₆R¹⁴, —(CR¹²R¹²)ₙC(O)NR¹²(CR¹²R¹²)₁₋₆R¹⁴, —(C(O)NR²⁷(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₆R¹⁴, C(O)(CR¹²R¹²)₁₋₆R¹⁴, and —C(O)C(R¹²R¹²R¹⁴).

In certain embodiments of the aforementioned compounds of Formula (I), R¹⁰ is selected from

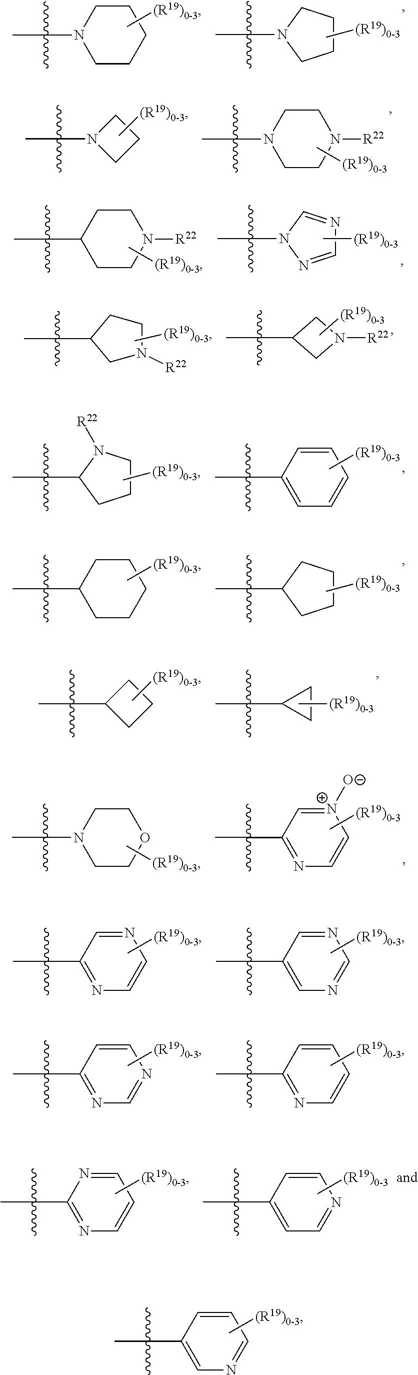

wherein each R¹⁹ is independently selected from halogen, hydroxyl, —NO₂, —CN, —C₁-C₆alkyl, —C₂-C₆alkene, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, R¹⁵, R¹¹, —OR¹², —OR¹¹, —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴, —(CR¹²R¹²)ₙC(O)NR¹²OR¹², —O(CR¹²R¹²)ₙR¹⁴, —O(CR¹²R¹²)ₙR¹¹, —(CR³R³)₁₋₄R¹⁴, —(CR¹²R¹²)ₙC(O)NR¹²(CR¹²R¹²)₁₋₆R¹⁴, —C(O)NR²⁷(CR¹²R¹²)ₙR¹¹, —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₄R¹⁴, —C(O)(CR¹²R¹²)₁₋₄R¹⁴, and —C(O)C(R¹²R¹²R¹⁴); R²² is H, —C₁-C₆alkyl, hydroxyl-C₁-C₆alkyl, —C(O)R¹², —C(O)R¹¹, R¹¹, —C(O)R¹⁵, —(CR¹²R¹²)₁₋₄R¹¹, —(CR¹²R¹²)₁₋₆R¹⁴,

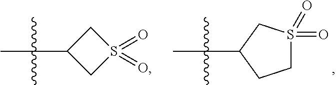

—(CR¹²R¹²)ₙC(O)N(R¹²R¹²), —(CR¹²R¹²)ₙC(O)NR¹²(CR¹²R¹²)₁₋₆R¹⁴, —C(O)C(R¹²R¹⁴)(C(R¹²R¹²))ₙR¹⁴, —(CR¹²R¹²)ₙC(O)NR¹²OR¹², —(CR¹²R¹²)ₙC(R¹²R¹⁴)(C(R¹²R¹²))ₙR¹⁴, —(CR¹²R¹²)ₙC(O)OR¹², —(CR¹²R¹²)ₙC(O)R¹¹ or —(CR¹²R¹²)ₙC(O)(CR¹²R¹²)₁₋₆R¹⁴, and each n is independently 0, 1, 2, 3 or 4.

In certain embodiments of the aforementioned compounds of Formula (I), R¹⁰ is —(CR¹²R¹²)ₙR¹¹.

In certain embodiments of the aforementioned compounds of Formula (I), R¹¹ is a 4-8 membered heterocycloalkyl containing 1 to 2 heteroatoms independently selected from N, O and S or a 4-8 membered heterocycloalkyl containing 1 to 2 heteroatoms independently selected from N, O and S substituted with 1 to 3 substituents independently selected from halogen, hydroxyl, —C₁-C₆alkyl, C₃-C₈cycloalkyl, hydroxyl-C₁-C₆alkyl and —(CR¹²R¹²)ₙR¹⁴, and each n is independently 0, 1, 2, 3, 4, 5 or 6.

In certain embodiments of the aforementioned compounds of Formula (I), R¹¹ is selected from

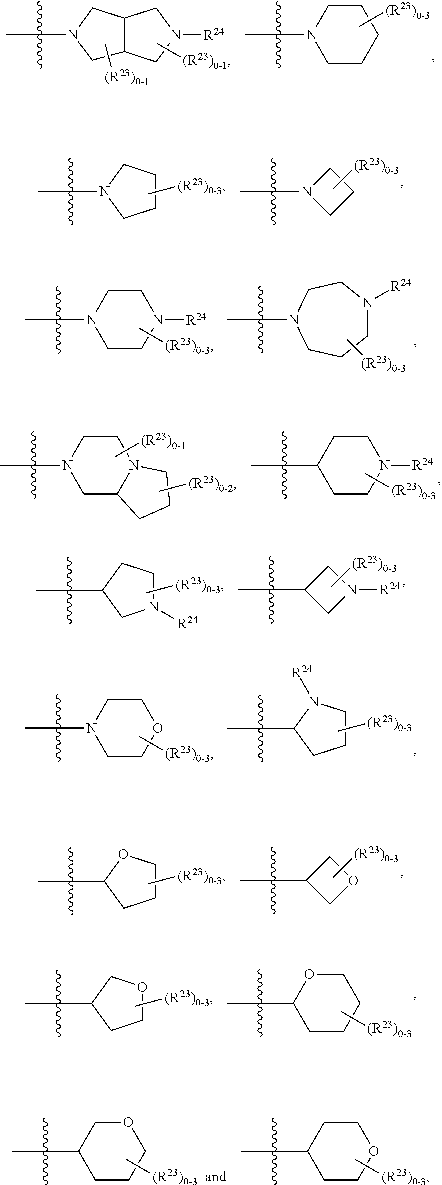

wherein each R²³ is independently selected from halogen, hydroxyl, —C₁-C₆alkyl, C₃-C₈cycloalkyl, hydroxyl-C₁-C₆alkyl and —(CR¹²R¹²)ₙR¹⁴; R²⁴ is H, C₁-C₆alkyl, hydroxyl-C₁-C₆alkyl or —(CR¹²R¹²)₁₋₄R¹⁴, and each n is independently 0, 1, 2, 3 or 4.

In certain embodiments of the aforementioned compounds of Formula (I), R¹¹ is a C₃-C₈cycloalkyl or a C₃-C₈cycloalkyl substituted with 1 to 3 substituents independently selected from halogen, hydroxyl, —C₁-C₆alkyl, C₃-C₈cycloalkyl, hydroxyl-C₁-C₆alkyl and —(CR¹²R¹²)ₙR¹⁴ and each n is independently 0, 1, 2, 3, 4, 5 or 6.

In certain embodiments of the aforementioned compounds of Formula (I), R¹¹ is selected from

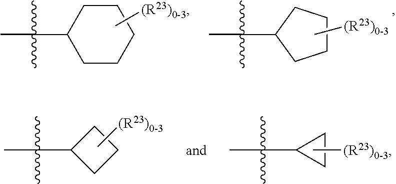

wherein, each R²³ is independently selected from halogen, hydroxyl, —C₁-C₆alkyl, C₃-C₈cycloalkyl, hydroxyl-C₁-C₆alkyl and —(CR¹²R¹²)ₙR¹⁴, and each n is independently 0, 1, 2, 3 or 4.

In certain embodiments of the aforementioned compounds of Formula (I), R¹¹ is a 5, 6 or 9 membered heteroaryl containing 1 to 4 heteroatoms independently selected from N, O and S, or a 5, 6 or 9 membered heteroaryl containing 1 to 4 heteroatoms independently selected from N, O and S substituted with 1 to 3 substituents independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴ and each n is independently 0, 1, 2, 3, 4, 5 or 6.

In certain embodiments of the aforementioned compounds of Formula (I), R¹¹ is selected from

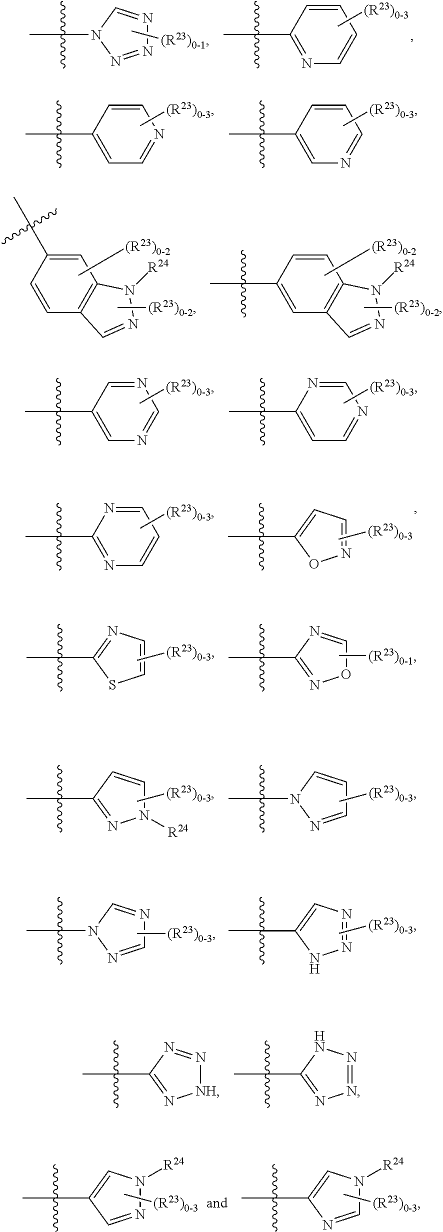

wherein each R²³ is independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴; R²⁴ is H, —C₁-C₆alkyl, hydroxyl-C₁-C₆alkyl or —(CR¹²R¹²)₁₋₄R¹⁴; and each n is independently 1, 2, 3 or 4.

In certain embodiments of the aforementioned compounds of Formula (I), R¹¹ is

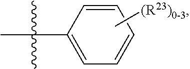

wherein each R²³ is independently selected from halogen, hydroxyl, —C₁-C₆alkyl, C₃-C₈cycloalkyl, hydroxyl-C₁-C₆alkyl and —(CR¹²R¹²)ₙR¹⁴, and each n is independently 0, 1, 2, 3 or 4.

In certain embodiments of the aforementioned compounds of Formula (I), R¹⁴ is selected from H, halogen, hydroxyl, hydroxyl-C₁-C₆alkyl, R¹³, —OR¹³, —OR¹², —O(CR¹²R¹²)ₙOR¹³, —C(O)R¹³, —N(R¹²)₂, —NR¹²OR¹³, —CN, —C(O)N(R¹²)₂, —S(O)₂R¹³—C(O)OR¹³, —S(O)₂N(R¹²)₂, —N(R¹²R¹⁰), —N(R¹²R¹¹), —(CR¹²R¹²)ₙR¹³, —N(R¹²)(CR¹²R¹²)ₙOR¹³, —C(O)N(R¹²)₂, and R¹⁵.

In certain embodiments of the aforementioned compounds of Formula (I), R³, R⁵ and R²⁶ are H.

In certain embodiments of the aforementioned compounds of Formula (I), R⁴ is H, C₁-C₆alkyl, deuterated C₁-C₆alkyl, C₁-C₆haloalkyl, C₂-C₆alkene, or —CD₃.

In certain embodiments of the aforementioned compounds of Formula (I), R⁴ is hydroxyl-C₁-C₆alkyl.

In certain embodiments of the aforementioned compounds of Formula (I), R⁴ is —(CR²⁷R²⁷)₁₋₆R¹⁴, —(CR²⁷R²⁷)(CR²⁷R²⁵)R¹¹, —(CR²⁷R²⁷)(CR²⁷R²⁵)R²⁵, —C(R²⁷R²⁵R²⁵) or —(CR²⁷R²⁷)ₙR¹¹.

In certain embodiments of the aforementioned compounds of Formula (I), each R²⁵ is independently selected from H, hydroxyl, and hydroxyl-C₁-C₆alkyl.

In certain embodiments the compounds of Formula (I) are selected from: 8-amino-2-methyl-6-(pyridin-2-ylamino)-1,2-dihydro-2,7-naphthyridin-1-one; 6-[(1-amino-7-methyl-8-oxo-7,8-dihydro-2,7-naphthyridin-3-yl)amino]pyridine-3-carbonitrile; 6-[(1-amino-7-methyl-8-oxo-7,8-dihydro-2,7-naphthyridin-3-yl)amino]pyridine-3-carboxamide; 8-amino-2-methyl-6-(pyrazin-2-ylamino)-1,2-dihydro-2,7-naphthyridin-1-one; 8-amino-2-benzyl-6-(pyrazin-2-ylamino)-1,2-dihydro-2,7-naphthyridin-1-one; 6-{3-methyl-1H-pyrazolo[3,4-b]pyridin-5-yl}-8-{[4-(morpholin-4-yl)phenyl]amino}-1,2-dihydro-2,7-naphthyridin-1-one; 8-{[3-fluoro-4-(2-methylpiperidin-4-yl)phenyl]amino}-6-(pyrimidin-5-yl)-1,2-dihydro-2,7-naphthyridin-1-one; 8-{[3-(4-ethylpiperazin-1-yl)-1H-indazol-6-yl]amino}-6-(pyrimidin-5-yl)-1,2-dihydro-2,7-naphthyridin-1-one; 8-{[3-(4-ethylpiperazin-1-yl)-1H-indazol-6-yl]amino}-6-(6-methylpyridin-3-yl)-1,2-dihydro-2,7-naphthyridin-1-one; 8-({3-[(4-ethylpiperazin-1-yl)methyl]-1H-indazol-6-yl}amino)-6-(pyrimidin-5-yl)-1,2-dihydro-2,7-naphthyridin-1-one; 8-({3-[(4-ethylpiperazin-1-yl)methyl]-1H-indazol-6-yl}amino)-6-(6-methylpyridin-3-yl)-1,2-dihydro-2,7-naphthyridin-1-one; 8-({3-[(4-ethylpiperazin-1-yl)methyl]-1H-indazol-6-yl}amino)-6-(2-methoxypyrimidin-5-yl)-1,2-dihydro-2,7-naphthyridin-1-one; 3-[4-(2-methyl-4-{[3-(6-methylpyridin-3-yl)-8-oxo-7,8-dihydro-2,7-naphthyridin-1-yl]amino}phenyl)piperidin-1-yl]propanenitrile; 6-(6-methylpyridin-3-yl)-8-({1-[2-(morpholin-4-yl)ethyl]-1H-pyrazol-4-yl}amino)-1,2-dihydro-2,7-naphthyridin-1-one; 8-({4-[1-(2,3-dihydroxypropyl)piperidin-4-yl]phenyl}amino)-6-(6-methoxypyrazin-2-yl)-1,2-dihydro-2,7-naphthyridin-1-one; 3-[4-(4-{[3-(6-methoxypyrazin-2-yl)-8-oxo-7,8-dihydro-2,7-naphthyridin-1-yl]amino}-2-methylphenyl)piperidin-1-yl]propanenitrile; 8-{[4-(morpholin-4-yl)phenyl]amino}-6-(pyrazin-2-ylamino)-1,2-dihydro-2,7-naphthyridin-1-one; 8-{[4-(morpholin-4-yl)phenyl]amino}-6-(pyridin-2-ylamino)-1,2-dihydro-2,7-naphthyridin-1-one; 6-[(3-methylpyridin-2-yl)amino]-8-{[4-(morpholin-4-yl)phenyl]amino}-1,2-dihydro-2,7-naphthyridin-1-one; 6-[(5-methylpyridin-2-yl)amino]-8-{[4-(morpholin-4-yl)phenyl]amino}-1,2-dihydro-2,7-naphthyridin-1-one; 6-[(6-methylpyridin-2-yl)amino]-8-{[4-(morpholin-4-yl)phenyl]amino}-1,2-dihydro-2,7-naphthyridin-1-one; 6-(6-methoxypyrazin-2-yl)-8-[(4-{2-[4-(propan-2-yl)piperazin-1-yl]ethyl}-phenyl)amino]-1,2-dihydro-2,7-naphthyridin-1-one; 8-{[4-(2-{6,9-diazaspiro[4.5]decan-9-yl}ethyl)phenyl]amino}-6-(6-methoxypyrazin-2-yl)-1,2-dihydro-2,7-naphthyridin-1-one; 8-({4-[2-(4-methoxypiperidin-1-yl)ethyl]phenyl}amino)-6-(6-methoxypyrazin-2-yl)-1,2-dihydro-2,7-naphthyridin-1-one; 6-(6-methoxypyrazin-2-yl)-8-({4-[2-(1H-1,2,4-triazol-1-yl)ethyl]phenyl}amino)-1,2-dihydro-2,7-naphthyridin-1-one; 6-(6-methoxypyrazin-2-yl)-8-({4-[3-(morpholin-4-yl)-3-oxopropyl]phenyl}amino)-1,2-dihydro-2,7-naphthyridin-1-one; 8-({4-[1-(3-methoxypropanoyl)piperidin-4-yl]phenyl}amino)-6-(6-methoxypyrazin-2-yl)-1,2-dihydro-2,7-naphthyridin-1-one; 8-[(4-{1-[2-(2-methoxyethoxy)acetyl]piperidin-4-yl}phenyl)amino]-6-(6-methoxypyrazin-2-yl)-1,2-dihydro-2,7-naphthyridin-1-one; 8-({4-[1-(2-ethoxyacetyl)piperidin-4-yl]phenyl}amino)-6-(6-methoxypyrazin-2-yl)-1,2-dihydro-2,7-naphthyridin-1-one; 2-{4-[(3-imidazo[pyrimidin-6-yl]-8-oxo-7,8-dihydro-2,7-naphthyridin-1-yl)amino]phenyl}-N,2-dimethylpropanamide; 6-{imidazo[1,2-a]pyrimidin-6-yl}-8-({4-[1-(2-methoxyethyl)piperidin-4-yl]-3-methylphenyl}amino)-1,2-dihydro-2,7-naphthyridin-1-one; 6-(2-aminopyrimidin-5-yl)-2-methyl-8-[(1-methylpiperidin-4-yl)amino]-1,2-dihydro-2,7-naphthyridin-1-one; 6-(2-aminopyrimidin-5-yl)-2-(2-hydroxyethyl)-8-(propan-2-ylamino)-1,2-dihydro-2,7-naphthyridin-1-one; 8-({3-fluoro-4-[1-(3-methoxypropyl)piperidin-4-yl]phenyl}amino)-6-(pyrimidin-5-yl)-1,2-dihydro-2,7-naphthyridin-1-one; 8-{[3-fluoro-4-(piperidin-4-yl)phenyl]amino}-6-(pyrimidin-5-yl)-1,2-dihydro-2,7-naphthyridin-1-one; 8-({3-fluoro-4-[1-(3-methoxypropyl)piperidin-4-yl]phenyl}amino)-6-(2-methoxypyrimidin-5-yl)-1,2-dihydro-2,7-naphthyridin-1-one; 8-{[3-fluoro-4-(piperidin-4-yl)phenyl]amino}-6-(2-methoxypyrimidin-5-yl)-1,2-dihydro-2,7-naphthyridin-1-one; 8-{[3-(4-ethylpiperazin-1-yl)-1H-indazol-6-yl]amino}-6-{5-[(morpholin-4-yl)carbonyl]pyridin-3-yl}-1,2-dihydro-2,7-naphthyridin-1-one; 8-({3-[(4-ethylpiperazin-1-yl)methyl]-1H-indazol-6-yl}amino)-6-{5-[(morpholin-4-yl)carbonyl]pyridin-3-yl}-1,2-dihydro-2,7-naphthyridin-1-one; 8-{[4-(1-cyclopropylpiperidin-4-yl)-3-methylphenyl]amino}-6-{5-[(morpholin-4-yl)carbonyl]pyridin-3-yl}-1,2-dihydro-2,7-naphthyridin-1-one; 8-({3-[(dimethylamino)methyl]-1H-indazol-6-yl}amino)-6-{5-[(morpholin-4-yl)carbonyl]pyridin-3-yl}-1,2-dihydro-2,7-naphthyridin-1-one; 8-({4-[2-(4-ethyl-3-methylpiperazin-1-yl)ethyl]phenyl}amino)-6-(6-methoxypyrazin-2-yl)-1,2-dihydro-2,7-naphthyridin-1-one; 6-(pyrimidin-5-yl)-8-({4-[2-(1H-1,2,4-triazol-1-yl)ethyl]phenyl}amino)-1,2-dihydro-2,7-naphthyridin-1-one; 6-(2-aminopyrimidin-5-yl)-8-(propan-2-ylamino)-1,2-dihydro-2,7-naphthyridin-1-one; 6-(2-aminopyrimidin-5-yl)-2-methyl-8-{[2-(morpholin-4-yl)ethyl]amino}-1,2-dihydro-2,7-naphthyridin-1-one; 6-(2-aminopyrimidin-5-yl)-8-(cyclopropylamino)-2-ethyl-1,2-dihydro-2,7-naphthyridin-1-one; 6-(2-aminopyrimidin-5-yl)-8-(cyclopropylamino)-2-methyl-1,2-dihydro-2,7-naphthyridin-1-one; 6-[(1-amino-7-methyl-8-oxo-7,8-dihydro-2,7-naphthyridin-3-yl)amino]-N-propylpyridine-3-carboxamide; 6-(3,6-dimethylpyrazin-2-yl)-8-{[4-(morpholin-4-yl)phenyl]amino}-1,2-dihydro-2,7-naphthyridin-1-one; 6-(6-methoxypyrazin-2-yl)-8-({4-[3-(morpholin-4-yl)propyl]phenyl}amino)-1,2-dihydro-2,7-naphthyridin-1-one; 8-({4-[3-(4-ethylpiperazin-1-yl)propyl]phenyl}amino)-6-(6-methoxypyrazin-2-yl)-1,2-dihydro-2,7-naphthyridin-1-one; 3-[4-(4-{[3-(6-methoxypyrazin-2-yl)-8-oxo-7,8-dihydro-2,7-naphthyridin-1-yl]amino}-2-methylphenyl)piperidin-1-yl]-1$1̂{6}-thietane-1,1-dione; 8-{[4-(azetidin-3-yl)phenyl]amino}-6-(pyrimidin-5-yl)-1,2-dihydro-2,7-naphthyridin-1-one; 8-{[4-(azetidin-3-yl)phenyl]amino}-6-(2-methoxypyrimidin-5-yl)-1,2-dihydro-2,7-naphthyridin-1-one; 8-{[4-(azetidin-3-yl)phenyl]amino}-6-(6-methylpyridin-3-yl)-1,2-dihydro-2,7-naphthyridin-1-one; 8-({4-[1-(2-methane sulfonylethyl)piperidin-4-yl]-3-methylphenyl}amino)-6-(6-methoxypyrazin-2-yl)-1,2-dihydro-2,7-naphthyridin-1-one; 8-({4-[1-(3-methoxypropanoyl)piperidin-4-yl]-3-methylphenyl}amino)-6-(6-methoxypyrazin-2-yl)-1,2-dihydro-2,7-naphthyridin-1-one; 8-({4-[2-(4,4-difluoropiperidin-1-yl)ethyl]phenyl}amino)-6-(6-methoxypyrazin-2-yl)-1,2-dihydro-2,7-naphthyridin-1-one; 8-({4-[2-(4-acetylpiperazin-1-yl)ethyl]phenyl}amino)-6-(6-methoxypyrazin-2-yl)-1,2-dihydro-2,7-naphthyridin-1-one; 2-[4-(4-{[3-(6-methoxypyrazin-2-yl)-8-oxo-7,8-dihydro-2,7-naphthyridin-1-yl]amino}-2-methylphenyl)piperidin-1-yl]acetonitrile; 2-[4-(4-{[3-(6-methoxypyrazin-2-yl)-8-oxo-7,8-dihydro-2,7-naphthyridin-1-yl]amino}-2-methylphenyl)piperidin-1-yl]acetamide; 2-[4-(4-{[3-(6-methoxypyrazin-2-yl)-8-oxo-7,8-dihydro-2,7-naphthyridin-1-yl]amino}-2-methylphenyl)piperidin-1-yl]-N-methylacetamide; 2-[4-(4-{[3-(6-methoxypyrazin-2-yl)-8-oxo-7,8-dihydro-2,7-naphthyridin-1-yl]amino}-2-methylphenyl)piperidin-1-yl]propanamide; 8-{[4-(1-ethylpiperidin-4-yl)-3-methylphenyl]amino}-6-(6-methoxypyrazin-2-yl)-1,2-dihydro-2,7-naphthyridin-1-one; 8-{[4-(1-methylazetidin-3-yl)phenyl]amino}-6-(pyrimidin-5-yl)-1,2-dihydro-2,7-naphthyridin-1-one; 6-(2-methoxypyrimidin-5-yl)-8-{[4-(1-methylazetidin-3-yl)phenyl]amino}-1,2-dihydro-2,7-naphthyridin-1-one; 8-{[4-(1-methylazetidin-3-yl)phenyl]amino}-6-(6-methylpyridin-3-yl)-1,2-dihydro-2,7-naphthyridin-1-one; 6-[(5-fluoropyridin-2-yl)amino]-8-{[4-(morpholin-4-yl)phenyl]amino}-1,2-dihydro-2,7-naphthyridin-1-one; 8-{[4-(morpholin-4-yl)phenyl]amino}-6-{[5-(trifluoromethyl)pyridin-2-yl]amino}-1,2-dihydro-2,7-naphthyridin-1-one; 6-[(1-{[4-(morpholin-4-yl)phenyl]amino}-8-oxo-7,8-dihydro-2,7-naphthyridin-3-yl)amino]pyridine-3-carbonitrile; 6-[(1-{[4-(morpholin-4-yl)phenyl]amino}-8-oxo-7,8-dihydro-2,7-naphthyridin-3-yl)amino]pyridine-3-carboxamide; 8-{[4-(morpholin-4-yl)phenyl]amino}-6-[(5-nitropyridin-2-yl)amino]-1,2-dihydro-2,7-naphthyridin-1-one; 8-({3-fluoro-4-[1-(2-methoxyethyl)piperidin-4-yl]phenyl}amino)-6-(pyrimidin-5-yl)-1,2-dihydro-2,7-naphthyridin-1-one; 8-{[3-(4-ethylpiperazin-1-yl)-1H-indazol-6-yl]amino}-6-(6-methoxypyrazin-2-yl)-1,2-dihydro-2,7-naphthyridin-1-one; 8-{[4-(1-cyclopropylpiperidin-4-yl)-3-methylphenyl]amino}-6-(6-methoxypyrazin-2-yl)-1,2-dihydro-2,7-naphthyridin-1-one; ethyl 6-[(1-{[4-(morpholin-4-yl)phenyl]amino}-8-oxo-7,8-dihydro-2,7-naphthyridin-3-yl)amino]pyridine-3-carboxylate; 6-[(5-chloropyridin-2-yl)amino]-8-{[4-(morpholin-4-yl)phenyl]amino}-1,2-dihydro-2,7-naphthyridin-1-one; 6-({5-[2-(morpholin-4-yl)ethyl]pyridin-2-yl}amino)-8-{[4-(morpholin-4-yl)phenyl]amino}-1,2-dihydro-2,7-naphthyridin-1-one; 6-(2-aminopyrimidin-5-yl)-4-fluoro-8-{[4-(morpholin-4-yl)phenyl]amino}-1,2-dihydro-2,7-naphthyridin-1-one; 4-fluoro-8-{[4-(morpholin-4-yl)phenyl]amino}-6-(pyrimidin-5-yl)-1,2-dihydro-2,7-naphthyridin-1-one; 4-fluoro-6-(2-methoxypyrimidin-5-yl)-8-{[4-(morpholin-4-yl)phenyl]amino}-1,2-dihydro-2,7-naphthyridin-1-one; 4-fluoro-6-(6-methylpyridin-3-yl)-8-{[4-(morpholin-4-yl)phenyl]amino}-1,2-dihydro-2,7-naphthyridin-1-one; 4-fluoro-6-(6-methoxypyrazin-2-yl)-8-{[4-(morpholin-4-yl)phenyl]amino}-1,2-dihydro-2,7-naphthyridin-1-one; 8-({4-[1-(3-methoxypropyl)piperidin-4-yl]-3-methylphenyl}amino)-6-(6-methoxypyrazin-2-yl)-1,2-dihydro-2,7-naphthyridin-1-one; 8-({4-[1-(2-hydroxy-3-methoxypropyl)piperidin-4-yl]-3-methylphenyl}amino)-6-(6-methoxypyrazin-2-yl)-1,2-dihydro-2,7-naphthyridin-1-one; 8-({4-[1-(2-hydroxyethyl)piperidin-4-yl]-3-methylphenyl}amino)-6-(6-methoxypyrazin-2-yl)-1,2-dihydro-2,7-naphthyridin-1-one; 8-{[4-(1-acetylpiperidin-4-yl)-3-methylphenyl]amino}-6-(6-methoxypyrazin-2-yl)-1,2-dihydro-2,7-naphthyridin-1-one; 8-{[4-(azetidin-3-yl)phenyl]amino}-6-(6-methoxypyrazin-2-yl)-1,2-dihydro-2,7-naphthyridin-1-one; 6-(6-methoxypyrazin-2-yl)-8-{[4-(1-methylazetidin-3-yl)phenyl]amino}-1,2-dihydro-2,7-naphthyridin-1-one; 8-({4-[1-(3-fluoropropyl)piperidin-4-yl]-3-methylphenyl}amino)-6-(6-methylpyridin-3-yl)-1,2-dihydro-2,7-naphthyridin-1-one; 8-({4-[1-(2-hydroxyethyl)azetidin-3-yl]phenyl}amino)-6-(6-methoxypyrazin-2-yl)-1,2-dihydro-2,7-naphthyridin-1-one; 8-({4-[1-(2-methanesulfonylethyl)azetidin-3-yl]phenyl}amino)-6-(6-methoxypyrazin-2-yl)-1,2-dihydro-2,7-naphthyridin-1-one; 8-{[4-(1-cyclopropylpiperidin-4-yl)-3-methylphenyl]amino}-6-(6-methylpyridin-3-yl)-1,2-dihydro-2,7-naphthyridin-1-one; 8-({4-[1-(2-methoxyethyl)piperidin-4-yl]-3-methylphenyl}amino)-6-(6-methylpyridin-3-yl)-1,2-dihydro-2,7-naphthyridin-1-one; 6-[6-(cyclopropylamino)pyrazin-2-yl]-8-{[4-(morpholin-4-yl)phenyl]amino}-1,2-dihydro-2,7-naphthyridin-1-one; 8-({4-[1-acetylazetidin-3-yl)phenyl]amino}-6-(6-methoxypyrazin-2-yl)-1,2-dihydro-2,7-naphthyridin-1-one; 8-({4-[1-(2-methoxyethyl)azetidin-3-yl]phenyl}amino)-6-(6-methoxypyrazin-2-yl)-1,2-dihydro-2,7-naphthyridin-1-one; 6-(2-methoxypyrimidin-5-yl)-8-({4-[2-(3-oxopiperazin-1-yl)ethyl]phenyl}amino)-1,2-dihydro-2,7-naphthyridin-1-one; 6-(6-methylpyridin-3-yl)-8-({4-[2-(3-oxopiperazin-1-yl)ethyl]phenyl}amino)-1,2-dihydro-2,7-naphthyridin-1-one; 3-[3-(4-{[3-(6-methoxypyrazin-2-yl)-8-oxo-7,8-dihydro-2,7-naphthyridin-1-yl]amino}phenyl)azetidin-1-yl]propanenitrile; 3-[4-(4-{[3-(5-amino-6-methoxypyrazin-2-yl)-8-oxo-7,8-dihydro-2,7-naphthyridin-1-yl]amino}-phenyl)piperidin-1-yl]propanenitrile; 8-({4-[1-(2-methoxyacetyl)piperidin-4-yl]-3-methylphenyl}amino)-6-(6-methoxypyrazin-2-yl)-1,2-dihydro-2,7-naphthyridin-1-one; 8-({4-[1-(2-ethoxyacetyl)piperidin-4-yl]-3-methylphenyl}amino)-6-(6-methoxypyrazin-2-yl)-1,2-dihydro-2,7-naphthyridin-1-one; 6-(2-amino-6-methylpyrimidin-4-yl)-8-({4-[1-(3-methoxypropanoyl)piperidin-4-yl]-3-methylphenyl}amino)-1,2-dihydro-2,7-naphthyridin-1-one; 8-({4-[1-(2-amino-2-methylpropanoyl)piperidin-4-yl]-3-methylphenyl}amino)-6-(6-methoxypyrazin-2-yl)-1,2-dihydro-2,7-naphthyridin-1-one; 6-(2-aminopyrimidin-4-yl)-8-{[4-(morpholin-4-yl)phenyl]amino}-1,2-dihydro-2,7-naphthyridin-1-one; 6-(6-methoxypyrazin-2-yl)-8-({4-[2-(piperazin-1-yl)ethyl]phenyl}amino)-1,2-dihydro-2,7-naphthyridin-1-one; 2-{4-[2-methyl-4-({3-[(5-methylpyridin-2-yl)amino]-8-oxo-7,8-dihydro-2,7-naphthyridin-1-yl}amino)phenyl]piperidin-1-yl}acetamide; 8-({4-[2-(4-acetylpiperazin-1-yl)ethyl]phenyl}amino)-6-[(5-methylpyridin-2-yl)amino]-1,2-dihydro-2,7-naphthyridin-1-one; 6-(6-methoxypyrazin-2-yl)-8-{[4-(2-{octahydropyrrolo[3,4-c]pyrrol-2-yl}ethyl)phenyl]amino}-1,2-dihydro-2,7-naphthyridin-1-one; 6-(6-methoxypyrazin-2-yl)-8-({4-[2-(4-methyl-1,4-diazepan-1-yl)ethyl]phenyl}amino)-1,2-dihydro-2,7-naphthyridin-1-one; 8-({4-[2-(4-methyl-1,4-diazepan-1-yl)ethyl]phenyl}amino)-6-[(5-methylpyridin-2-yl)amino]-1,2-dihydro-2,7-naphthyridin-1-one; 8-{[4-(1-cyclopropylpiperidin-4-yl)-3-methylphenyl]amino}-6-[1-(2-methylpropyl)-1H-pyrazol-4-yl]-1,2-dihydro-2,7-naphthyridin-1-one; 6-(6-methoxypyrazin-2-yl)-8-{[3-methyl-4-(1-propylpiperidin-4-yl)phenyl]amino}-1,2-dihydro-2,7-naphthyridin-1-one; 6-(6-methoxypyrazin-2-yl)-8-({4-[4-(morpholin-4-yl)cyclohexyl]phenyl}amino)-1,2-dihydro-2,7-naphthyridin-1-one; 6-(6-methoxypyrazin-2-yl)-8-({4-[2-(3-oxopiperazin-1-yl)ethyl]phenyl}amino)-1,2-dihydro-2,7-naphthyridin-1-one; 6-(6-methoxypyrazin-2-yl)-8-[(3-methyl-4-{1-[2-oxo-2-(pyrrolidin-1-yl)ethyl]piperidin-4-yl}phenyl)amino]-1,2-dihydro-2,7-naphthyridin-1-one; methyl 2-[4-(4-{[3-(6-methoxypyrazin-2-yl)-8-oxo-7,8-dihydro-2,7-naphthyridin-1-yl]amino}-2-methylphenyl)piperidin-1-yl]acetate; 3-[4-(4-{[3-(6-methoxypyrazin-2-yl)-8-oxo-7,8-dihydro-2,7-naphthyridin-1-yl]amino}-2-methylphenyl)piperidin-1-yl]propanamide; 3-[4-(4-{[3-(6-methoxypyrazin-2-yl)-8-oxo-7,8-dihydro-2,7-naphthyridin-1-yl]amino}-2-methylphenyl)piperidin-1-yl]-N,N-dimethylpropanamide; 6-[(5-methylpyridin-2-yl)amino]-8-{[4-(2-{octahydropyrrolo[3,4-c]pyrrol-2-yl}ethyl)phenyl]amino}-1,2-dihydro-2,7-naphthyridin-1-one; 8-({4-[2-(4-methyl-1,4-diazepan-1-yl)ethyl]phenyl}amino)-6-(pyrimidin-5-yl)-1,2-dihydro-2,7-naphthyridin-1-one; 6-(2-methoxypyrimidin-5-yl)-8-({4-[2-(4-methyl-1,4-diazepan-1-yl)ethyl]phenyl}amino)-1,2-dihydro-2,7-naphthyridin-1-one; 8-({4-[2-(4-methyl-1,4-diazepan-1-yl)ethyl]phenyl}amino)-6-(6-methylpyridin-3-yl)-1,2-dihydro-2,7-naphthyridin-1-one; 8-({4-[2-(4-ethylpiperazin-1-yl)ethyl]phenyl}amino)-6-[(5-methylpyridin-2-yl)amino]-1,2-dihydro-2,7-naphthyridin-1-one; 8-({4-[2-(4-ethylpiperazin-1-yl)ethyl]phenyl}amino)-6-(pyridin-2-ylamino)-1,2-dihydro-2,7-naphthyridin-1-one; 8-{[4-(1-cyclopropylpiperidin-4-yl)-3-methylphenyl]amino}-6-[(5-methylpyridin-2-yl)amino]-1,2-dihydro-2,7-naphthyridin-1-one; 6-[(5-chloropyridin-2-yl)amino]-8-{[4-(1-cyclopropylpiperidin-4-yl)-3-methylphenyl]amino}-1,2-dihydro-2,7-naphthyridin-1-one; 8-{[4-(1-cyclopropylpiperidin-4-yl)-3-methylphenyl]amino}-6-(2-methylpyridin-4-yl)-1,2-dihydro-2,7-naphthyridin-1-one; methyl 3-[4-(4-{[3-(6-methoxypyrazin-2-yl)-8-oxo-7,8-dihydro-2,7-naphthyridin-1-yl]amino}-2-methylphenyl)piperidin-1-yl]propanoate; 3-[4-(4-{[3-(6-methoxypyrazin-2-yl)-8-oxo-7,8-dihydro-2,7-naphthyridin-1-yl]amino}-2-methylphenyl)piperidin-1-yl]-N-methylpropanamide; 6-(6-methoxypyrazin-2-yl)-8-[(3-methyl-4-{1-[3-(morpholin-4-yl)-3-oxopropyl]piperidin-4-yl}phenyl)amino]-1,2-dihydro-2,7-naphthyridin-1-one; 3-[4-(4-{[3-(6-methoxypyrazin-2-yl)-8-oxo-7,8-dihydro-2,7-naphthyridin-1-yl]amino}-2-methylphenyl)piperidin-1-yl]propanoic acid; 8-{[4-(1-cyclopropylpiperidin-4-yl)-3-methylphenyl]amino}-6-[(5-methylpyrazin-2-yl)amino]-1,2-dihydro-2,7-naphthyridin-1-one; 8-({4-[4-(morpholin-4-yl)cyclohexyl]phenyl}amino)-6-(pyrimidin-5-yl)-1,2-dihydro-2,7-naphthyridin-1-one; 8-[(4-{1-[(2R)-2-methoxypropanoyl]piperidin-4-yl}-3-methylphenyl)amino]-6-(6-methoxypyrazin-2-yl)-1,2-dihydro-2,7-naphthyridin-1-one; 8-[(4-{1-[(2S)-2-methoxypropanoyl]piperidin-4-yl}-3-methylphenyl)amino]-6-(6-methoxypyrazin-2-yl)-1,2-dihydro-2,7-naphthyridin-1-one; N-(2-methoxyethyl)-4-{[3-(6-methoxypyrazin-2-yl)-8-oxo-7,8-dihydro-2,7-naphthyridin-1-yl]amino}benzamide; 6-{[1-({4-[2-(4-ethylpiperazin-1-yl)ethyl]phenyl}amino)-8-oxo-7,8-dihydro-2,7-naphthyridin-3-yl]amino}pyridine-3-carboxamide; 6-{[1-({4-[2-(4-ethylpiperazin-1-yl)ethyl]phenyl}amino)-8-oxo-7,8-dihydro-2,7-naphthyridin-3-yl]amino}pyridine-3-carbonitrile; 6-[(5-chloropyridin-2-yl)amino]-8-({4-[2-(4-ethylpiperazin-1-yl)ethyl]phenyl}amino)-1,2-dihydro-2,7-naphthyridin-1-one; 8-({4-[2-(4-ethylpiperazin-1-yl)ethyl]phenyl}amino)-4-fluoro-6-(pyrimidin-5-yl)-1,2-dihydro-2,7-naphthyridin-1-one; 8-({4-[2-(4-ethylpiperazin-1-yl)ethyl]phenyl}amino)-4-fluoro-6-(2-methoxypyrimidin-5-yl)-1,2-dihydro-2,7-naphthyridin-1-one; 8-({4-[2-(4-ethylpiperazin-1-yl)ethyl]phenyl}amino)-4-fluoro-6-(6-methoxypyrazin-2-yl)-1,2-dihydro-2,7-naphthyridin-1-one; 8-({4-[2-(4-ethylpiperazin-1-yl)ethyl]phenyl}amino)-6-(1-methyl-1H-pyrazol-4-yl)-1,2-dihydro-2,7-naphthyridin-1-one; 8-({4-[2-(4-ethylpiperazin-1-yl)ethyl]phenyl}amino)-4-fluoro-6-(1-methyl-1H-pyrazol-4-yl)-1,2-dihydro-2,7-naphthyridin-1-one; 8-({4-[2-(4-ethylpiperazin-1-yl)ethyl]phenyl}amino)-4-fluoro-6-(6-methylpyridin-3-yl)-1,2-dihydro-2,7-naphthyridin-1-one; 8-({4-[2-(4-ethylpiperazin-1-yl)ethyl]phenyl}amino)-6-[1-(2-methylpropyl)-1H-pyrazol-4-yl]-1,2-dihydro-2,7-naphthyridin-1-one; 8-({4-[2-(4-ethylpiperazin-1-yl)ethyl]phenyl}amino)-4-fluoro-6-[1-(2-methylpropyl)-1H-pyrazol-4-yl]-1,2-dihydro-2,7-naphthyridin-1-one; 8-({4-[2-(4-ethylpiperazin-1-yl)ethyl]phenyl}amino)-6-[(5-fluoropyridin-2-yl)amino]-1,2-dihydro-2,7-naphthyridin-1-one; 8-({4-[2-(4-ethylpiperazin-1-yl)ethyl]phenyl}amino)-6-{[5-(trifluoromethyl)pyridin-2-yl]amino}-1,2-dihydro-2,7-naphthyridin-1-one; 6-{[1-({4-[2-(4-ethylpiperazin-1-yl)ethyl]phenyl}amino)-8-oxo-7,8-dihydro-2,7-naphthyridin-3-yl]amino}pyridine-3-carboxylic acid; 8-({3-methyl-4-[(2R)-2-methylmorpholin-4-yl]phenyl}amino)-6-[(5-methylpyridin-2-yl)amino]-1,2-dihydro-2,7-naphthyridin-1-one; 6-{[1-({3-methyl-4-[(2R)-2-methylmorpholin-4-yl]phenyl}amino)-8-oxo-7,8-dihydro-2,7-naphthyridin-3-yl]amino}pyridine-3-carboxamide; 6-{[1-({3-methyl-4-[(2R)-2-methylmorpholin-4-yl]phenyl}amino)-8-oxo-7,8-dihydro-2,7-naphthyridin-3-yl]amino}pyridine-3-carbonitrile; 8-({3-methyl-4-[(2R)-2-methylmorpholin-4-yl]phenyl}amino)-6-{[5-(trifluoromethyl)pyridin-2-yl]amino}-1,2-dihydro-2,7-naphthyridin-1-one; 8-[(4-{1-[(1-aminocyclopropyl)carbonyl]piperidin-4-yl}-3-methylphenyl)amino]-6-(6-methoxypyrazin-2-yl)-1,2-dihydro-2,7-naphthyridin-1-one; 8-({4-[1-(3,3-dimethylbutyl)piperidin-4-yl]-3-methylphenyl}amino)-6-(6-methoxypyrazin-2-yl)-1,2-dihydro-2,7-naphthyridin-1-one; 8-{[4-(1-cyclopentylpiperidin-4-yl)-3-methylphenyl]amino}-6-(6-methoxypyrazin-2-yl)-1,2-dihydro-2,7-naphthyridin-1-one; 8-[(4-{1-[2-(dimethylamino)ethyl]piperidin-4-yl}-3-methylphenyl)amino]-6-(6-methoxypyrazin-2-yl)-1,2-dihydro-2,7-naphthyridin-1-one; 8-({4-[(4-ethylpiperazin-1-yl)carbonyl]phenyl}amino)-6-(6-methoxypyrazin-2-yl)-1,2-dihydro-2,7-naphthyridin-1-one; 3-[3-(4-{[3-(6-methoxypyrazin-2-yl)-8-oxo-7,8-dihydro-2,7-naphthyridin-1-yl]amino}-phenyl)pyrrolidin-1-yl]propanenitrile; 3-{4-[2-(4-{[3-(6-methoxypyrazin-2-yl)-8-oxo-7,8-dihydro-2,7-naphthyridin-1-yl]amino}-phenyl)ethyl]piperazin-1-yl}propanenitrile; 3-[4-(4-{[3-(2-aminopyrimidin-4-yl)-8-oxo-7,8-dihydro-2,7-naphthyridin-1-yl]amino}-2-methylphenyl)piperidin-1-yl]propanenitrile; 3-{4-[4-({3-[6-(cyclopropylamino)pyrazin-2-yl]-8-oxo-7,8-dihydro-2,7-naphthyridin-1-yl}amino)-2-methylphenyl]piperidin-1-yl}propanenitrile; 6-(6-methoxypyrazin-2-yl)-8-({3-methyl-4-[1-(oxetan-3-yl)piperidin-4-yl]phenyl}amino)-1,2-dihydro-2,7-naphthyridin-1-one; 3-[4-(4-{[3-(6-amino-5-methylpyridin-3-yl)-8-oxo-7,8-dihydro-2,7-naphthyridin-1-yl]amino}-2-methylphenyl)piperidin-1-yl]propanenitrile; 6-(6-methoxypyrazin-2-yl)-8-({3-methyl-4-[1-(propan-2-yl)piperidin-4-yl]phenyl}amino)-1,2-dihydro-2,7-naphthyridin-1-one; 3-[4-(4-{[3-(2-amino-6-methylpyrimidin-4-yl)-8-oxo-7,8-dihydro-2,7-naphthyridin-1-yl]amino}-2-methylphenyl)piperidin-1-yl]propanenitrile; 8-({4-[1-(2,3-dihydroxypropyl)piperidin-4-yl]-3-methylphenyl}amino)-6-(6-methoxypyrazin-2-yl)-1,2-dihydro-2,7-naphthyridin-1-one; 2-[4-(4-{[3-(6-methoxypyrazin-2-yl)-8-oxo-7,8-dihydro-2,7-naphthyridin-1-yl]amino}-2-methylphenyl)piperidin-1-yl]-N,N-dimethylacetamide; 6-(5-amino-6-methoxypyrazin-2-yl)-8-{[3-methyl-4-(piperidin-4-yl)phenyl]amino}-1,2-dihydro-2,7-naphthyridin-1-one; 3-[4-(4-{[3-(5-amino-6-methoxypyrazin-2-yl)-8-oxo-7,8-dihydro-2,7-naphthyridin-1-yl]amino}-2-methylphenyl)piperidin-1-yl]propanenitrile; 6-[(1-{[4-(1-cyclopropylpiperidin-4-yl)-3-methylphenyl]amino}-8-oxo-7,8-dihydro-2,7-naphthyridin-3-yl)amino]pyridine-3-carboxamide; 8-{[4-(1-cyclopropylpiperidin-4-yl)-3-methylphenyl]amino}-6-(3,4-dimethoxyphenyl)-1,2-dihydro-2,7-naphthyridin-1-one; 8-({3-[4-(2-methoxyethyl)piperazin-1-yl]-1H-indazol-6-yl}amino)-6-(pyrimidin-5-yl)-1,2-dihydro-2,7-naphthyridin-1-one; 8-({3-[4-(2-methoxyethyl)piperazin-1-yl]-1H-indazol-6-yl}amino)-6-(6-methylpyridin-3-yl)-1,2-dihydro-2,7-naphthyridin-1-one; 8-({3-[4-(2-methoxyethyl)piperazin-1-yl]-1H-indazol-6-yl}amino)-6-(2-methoxypyrimidin-5-yl)-1,2-dihydro-2,7-naphthyridin-1-one; 8-{[4-(1-cyclopropylpiperidin-4-yl)-3-methylphenyl]amino}-6-[(4-methylpyridin-2-yl)amino]-1,2-dihydro-2,7-naphthyridin-1-one; 8-{[4-(1-cyclopropylpiperidin-4-yl)-3-methylphenyl]amino}-6-[(4-ethylpyridin-2-yl)amino]-1,2-dihydro-2,7-naphthyridin-1-one; 8-{[4-(1-cyclopropylpiperidin-4-yl)-3-methylphenyl]amino}-6-[(4-propylpyridin-2-yl)amino]-1,2-dihydro-2,7-naphthyridin-1-one; 8-{[4-(1-cyclopropylpiperidin-4-yl)-3-methylphenyl]amino}-6-{[4-(trifluoromethyl)pyridin-2-yl]amino}-1,2-dihydro-2,7-naphthyridin-1-one; 8-{[4-(1-cyclopropylpiperidin-4-yl)-3-methylphenyl]amino}-6-[(4,6-dimethylpyridin-2-yl)amino]-1,2-dihydro-2,7-naphthyridin-1-one; 6-[(1-{[4-(1-cyclopropylpiperidin-4-yl)-3-methylphenyl]amino}-8-oxo-7,8-dihydro-2,7-naphthyridin-3-yl)amino]pyridine-3-sulfonamide; 8-({3-methyl-4-[(2R)-2-methylmorpholin-4-yl]phenyl}amino)-6-[(4-methylpyridin-2-yl)amino]-1,2-dihydro-2,7-naphthyridin-1-one; 8-({4-[2-(4-ethylpiperazin-1-yl)ethyl]phenyl}amino)-6-[(4-methylpyridin-2-yl)amino]-1,2-dihydro-2,7-naphthyridin-1-one; 8-({4-[2-(4-ethylpiperazin-1-yl)ethyl]phenyl}amino)-6-[(4-ethylpyridin-2-yl)amino]-1,2-dihydro-2,7-naphthyridin-1-one; 8-({4-[2-(4-ethylpiperazin-1-yl)ethyl]phenyl}amino)-6-{[4-(trifluoromethyl)pyridin-2-yl]amino}-1,2-dihydro-2,7-naphthyridin-1-one; 2-{[1-({4-[2-(4-ethylpiperazin-1-yl)ethyl]phenyl}amino)-8-oxo-7,8-dihydro-2,7-naphthyridin-3-yl]amino}pyridine-4-carboxylic acid; 6-[(4,6-dimethylpyridin-2-yl)amino]-8-({4-[2-(4-ethylpiperazin-1-yl)ethyl]phenyl}amino)-1,2-dihydro-2,7-naphthyridin-1-one; 6-[(4-methylpyridin-2-yl)amino]-8-{[4-(morpholin-4-yl)phenyl]amino}-1,2-dihydro-2,7-naphthyridin-1-one; 6-(2-methylpyrimidin-5-yl)-8-({4-[4-(morpholin-4-yl)cyclohexyl]phenyl}amino)-1,2-dihydro-2,7-naphthyridin-1-one; 8-({4-[1-(2-methoxyethyl)piperidin-4-yl]-3-methylphenyl}amino)-6-(2-methylpyrimidin-5-yl)-1,2-dihydro-2,7-naphthyridin-1-one; 8-({4-[1-(3-fluoropropyl)piperidin-4-yl]-3-methylphenyl}amino)-6-(2-methylpyrimidin-5-yl)-1,2-dihydro-2,7-naphthyridin-1-one; 3-[4-(2-methyl-4-{[3-(2-methylpyrimidin-5-yl)-8-oxo-7,8-dihydro-2,7-naphthyridin-1-yl]amino}-phenyl)piperidin-1-yl]propanenitrile; N-(2-methoxyethyl)-4-{[3-(2-methylpyrimidin-5-yl)-8-oxo-7,8-dihydro-2,7-naphthyridin-1-yl]amino}benzamide; 6-(6-methoxypyrazin-2-yl)-8-[(3-methyl-4-{1-[(3-methyloxetan-3-yl)carbonyl]piperidin-4-yl}phenyl)amino]-1,2-dihydro-2,7-naphthyridin-1-one; 6-(6-methoxypyrazin-2-yl)-8-({3-methyl-4-[1-(2-methylbutanoyl)piperidin-4-yl]phenyl}amino)-1,2-dihydro-2,7-naphthyridin-1-one; 6-(6-methoxypyrazin-2-yl)-8-({3-methyl-4-[1-(oxolan-2-ylmethyl)piperidin-4-yl]phenyl}amino)-1,2-dihydro-2,7-naphthyridin-1-one; 8-[(4-{1-[(2,2-difluorocyclopropyl)methyl]piperidin-4-yl}-3-methylphenyl)amino]-6-(6-methoxypyrazin-2-yl)-1,2-dihydro-2,7-naphthyridin-1-one; 8-({4-[1-(2-fluoroethyl)piperidin-4-yl]-3-methylphenyl}amino)-6-(6-methoxypyrazin-2-yl)-1,2-dihydro-2,7-naphthyridin-1-one; 6-(6-methoxypyrazin-2-yl)-8-{[3-methyl-4-(1-propanoylpiperidin-4-yl)phenyl]amino}-1,2-dihydro-2,7-naphthyridin-1-one; 8-({4-[1-(cyclopropylmethyl)piperidin-4-yl]-3-methylphenyl}amino)-6-(6-methoxypyrazin-2-yl)-1,2-dihydro-2,7-naphthyridin-1-one; 4-{[3-(6-methoxypyrazin-2-yl)-8-oxo-7,8-dihydro-2,7-naphthyridin-1-yl]amino}-N-[2-(pyrrolidin-1-yl)ethyl]benzamide; N-{[(2R)-1-ethylpyrrolidin-2-yl]methyl}-4-{[3-(6-methoxypyrazin-2-yl)-8-oxo-7,8-dihydro-2,7-naphthyridin-1-yl]amino}benzamide; 6-(6-methoxypyrazin-2-yl)-8-[(3-methyl-4-{1-[2-methyl-2-(methylamino)propanoyl]piperidin-4-yl}phenyl)amino]-1,2-dihydro-2,7-naphthyridin-1-one; 8-({4-[1-(2-ethoxyethyl)piperidin-4-yl]-3-methylphenyl}amino)-6-(6-methoxypyrazin-2-yl)-1,2-dihydro-2,7-naphthyridin-1-one; 2-[4-(4-{[3-(6-methoxypyrazin-2-yl)-8-oxo-7,8-dihydro-2,7-naphthyridin-1-yl]amino}-2-methylphenyl)piperidin-1-yl]-N,N-dimethylpropanamide; 3-[4-(4-{[3-(6-methoxypyrazin-2-yl)-8-oxo-7,8-dihydro-2,7-naphthyridin-1-yl]amino}-2-methylphenyl)piperidin-1-yl]butanenitrile; 4-[4-(4-{[3-(6-methoxypyrazin-2-yl)-8-oxo-7,8-dihydro-2,7-naphthyridin-1-yl]amino}-2-methylphenyl)piperidin-1-yl]butanenitrile; 2-[4-(4-{[3-(6-methoxypyrazin-2-yl)-8-oxo-7,8-dihydro-2,7-naphthyridin-1-yl]amino}-2-methylphenyl)piperidin-1-yl]-N-methylpropanamide; 6-[(4-ethylpyridin-2-yl)amino]-8-(propan-2-ylamino)-1,2-dihydro-2,7-naphthyridin-1-one; 2-{[8-oxo-1-(propan-2-ylamino)-7,8-dihydro-2,7-naphthyridin-3-yl]amino}pyridine-4-carboxylic acid; 6-[(4-methylpyridin-2-yl)amino]-8-(propan-2-ylamino)-1,2-dihydro-2,7-naphthyridin-1-one; 6-[(4-chloropyridin-2-yl)amino]-8-(propan-2-ylamino)-1,2-dihydro-2,7-naphthyridin-1-one; 6-[(4-chloropyridin-2-yl)amino]-8-{[4-(1-cyclopropylpiperidin-4-yl)-3-methylphenyl]amino}-1,2-dihydro-2,7-naphthyridin-1-one; 8-{[4-(1-cyclopropylpiperidin-4-yl)-3-methylphenyl]amino}-6-[6-(dimethylamino)pyridin-3-yl]-1,2-dihydro-2,7-naphthyridin-1-one; 6-(3,6-dihydro-2H-pyran-4-yl)-8-{[4-(morpholin-4-yl)phenyl]amino}-1,2-dihydro-2,7-naphthyridin-1-one; 8-{[4-(morpholin-4-yl)phenyl]amino}-6-(oxan-4-yl)-1,2-dihydro-2,7-naphthyridin-1-one; 8-{[4-(morpholin-4-yl)phenyl]amino}-6-(1,2,3,6-tetrahydropyridin-4-yl)-1,2-dihydro-2,7-naphthyridin-1-one; 6-(6-methoxypyrazin-2-yl)-8-[(3-methyl-4-{1-[(oxolan-3-yl)carbonyl]piperidin-4-yl}phenyl)amino]-1,2-dihydro-2,7-naphthyridin-1-one; 8-({4-[1-(2-chloro-3-methoxypropanoyl)piperidin-4-yl]-3-methylphenyl}amino)-6-(6-methoxypyrazin-2-yl)-1,2-dihydro-2,7-naphthyridin-1-one; 8-({4-[1-(1-methoxypropan-2-yl)piperidin-4-yl]-3-methylphenyl}amino)-6-(6-methoxypyrazin-2-yl)-1,2-dihydro-2,7-naphthyridin-1-one; 2-[4-(4-{[3-(6-methoxypyrazin-2-yl)-8-oxo-7,8-dihydro-2,7-naphthyridin-1-yl]amino}-2-methylphenyl)piperidin-1-yl]-N-(propan-2-yl)acetamide; N-methoxy-2-[4-(4-{[3-(6-methoxypyrazin-2-yl)-8-oxo-7,8-dihydro-2,7-naphthyridin-1-yl]amino}-2-methylphenyl)piperidin-1-yl]-N-methylacetamide; 4-(4-[3-(6-methoxypyrazin-2-yl)-8-oxo-7,8-dihydro-2,7-naphthyridin-1-yl]amino-2-methylphenyl)-N,N-dimethylpiperidine-1-carboxamide; 3-[4-(4-{[3-(6-methoxypyrazin-2-yl)-8-oxo-7,8-dihydro-2,7-naphthyridin-1-yl]amino}-2-methylphenyl)piperidin-1-yl]-2-methylpropanenitrile; N-ethyl-2-[4-(4-{[3-(6-methoxypyrazin-2-yl)-8-oxo-7,8-dihydro-2,7-naphthyridin-1-yl]amino}-2-methylphenyl)piperidin-1-yl]acetamide; N,N-diethyl-2-[4-(4-{[3-(6-methoxypyrazin-2-yl)-8-oxo-7,8-dihydro-2,7-naphthyridin-1-yl]amino}-2-methylphenyl)piperidin-1-yl]acetamide; 6-(6-methoxypyrazin-2-yl)-8-[(3-methyl-4-{1-[(pyrrolidin-1-yl)carbonyl]piperidin-4-yl}phenyl)amino]-1,2-dihydro-2,7-naphthyridin-1-one; 6-(6-methoxypyrazin-2-yl)-8-[(3-methyl-4-{1-[(2-oxoimidazolidin-1-yl)carbonyl]piperidin-4-yl}phenyl)amino]-1,2-dihydro-2,7-naphthyridin-1-one; 6-(6-methoxypyrazin-2-yl)-8-[(3-methyl-4-{1-[(morpholin-4-yl)carbonyl]piperidin-4-yl}phenyl)amino]-1,2-dihydro-2,7-naphthyridin-1-one; methyl 2-[4-(4-{[3-(6-methoxypyrazin-2-yl)-8-oxo-7,8-dihydro-2,7-naphthyridin-1-yl]amino}-2-methylphenyl)piperidin-1-yl]propanoate; 8-({3-[4-(2-methoxyethyl)piperazin-1-yl]-1H-indazol-6-yl}amino)-6-(6-methoxypyrazin-2-yl)-1,2-dihydro-2,7-naphthyridin-1-one; 8-{[4-(1-cyclopropylpiperidin-4-yl)-3-methylphenyl]amino}-6-[(4-methoxypyridin-2-yl)amino]-1,2-dihydro-2,7-naphthyridin-1-one; 8-{[4-(1-cyclopropylpiperidin-4-yl)-3-methylphenyl]amino}-6-{[4-(hydroxymethyl)pyridin-2-yl]amino}-1,2-dihydro-2,7-naphthyridin-1-one; 8-{[4-(morpholin-4-yl)phenyl]amino}-6-(piperidin-4-yl)-1,2-dihydro-2,7-naphthyridin-1-one; 6-[1-(2-methoxyethyl)piperidin-4-yl]-8-[4-(morpholin-4-yl)phenyl]amino-1,2-dihydro-2,7-naphthyridin-1-one; 8-{[4-(morpholin-4-yl)phenyl]amino}-6-[1-(propan-2-yl)piperidin-4-yl]-1,2-dihydro-2,7-naphthyridin-1-one; 6-(2-aminopyrimidin-5-yl)-2-[(2,2-difluorocyclopropyl)methyl]-8-{[4-(morpholin-4-yl)phenyl]amino}-1,2-dihydro-2,7-naphthyridin-1-one; 6-acetyl-8-({3-methyl-4-[(2R)-2-methylmorpholin-4-yl]phenyl}amino)-1,2-dihydro-2,7-naphthyridin-1-one; 8-[(4-{2,6-diazaspiro[3.3]heptan-2-yl}phenyl)amino]-6-(6-methoxypyrazin-2-yl)-1,2-dihydro-2,7-naphthyridin-1-one; 2-[(1-[4-(1-cyclopropylpiperidin-4-yl)-3-methylphenyl]amino-8-oxo-7,8-dihydro-2,7-naphthyridin-3-yl)amino]pyridine-4-carboxylic acid; 8-({4-[1-(2-hydroxy-3-methylbutanoyl)piperidin-4-yl]-3-methylphenyl}amino)-6-(6-methoxypyrazin-2-yl)-1,2-dihydro-2,7-naphthyridin-1-one; 6-(6-methoxypyrazin-2-yl)-8-{[4-(2-{octahydropyrrolo[1,2-a]piperazin-2-yl}ethyl)phenyl]amino}-1,2-dihydro-2,7-naphthyridin-1-one; 6-acetyl-8-[4-(1-cyclopropylpiperidin-4-yl)-3-methylphenyl]amino-1,2-dihydro-2,7-naphthyridin-1-one; 8-({4-[1-(3-methoxy-2,2-dimethylpropanoyl)piperidin-4-yl]-3-methylphenyl}amino)-6-(6-methoxypyrazin-2-yl)-1,2-dihydro-2,7-naphthyridin-1-one; 8-{[4-(1-cyclopropylpiperidin-4-yl)-3-methylphenyl]amino}-6-[(4,6-dimethylpyridin-2-yl)amino]-1,2-dihydro-2,7-naphthyridin-1-one; 6-[(4-methylpyridin-2-yl)amino]-8-(propan-2-ylamino)-1,2-dihydro-2,7-naphthyridin-1-one; 8-({4-[4-(morpholin-4-yl)cyclohexyl]phenyl}amino)-6-(pyrimidin-5-yl)-1,2-dihydro-2,7-naphthyridin-1-one; 6-(2-methylpyrimidin-5-yl)-8-({4-[4-(morpholin-4-yl)cyclohexyl]phenyl}amino)-1,2-dihydro-2,7-naphthyridin-1-one; 6-(6-methoxypyrazin-2-yl)-8-({4-[4-(morpholin-4-yl)cyclohexyl]phenyl}amino)-1,2-dihydro-2,7-naphthyridin-1-one; 6-(2-ethoxyethyl)-8-({3-methyl-4-[(2R)-2-methylmorpholin-4-yl]phenyl}amino)-1,2-dihydro-2,7-naphthyridin-1-one; 8-{[4-(3,6-dihydro-2H-thiopyran-4-yl)-3-methylphenyl]amino}-6-(6-methoxypyrazin-2-yl)-1,2-dihydro-2,7-naphthyridin-1-one; 6-(6-methoxypyrazin-2-yl)-8-{[3-methyl-4-(1,2,3,6-tetrahydropyridin-4-yl)phenyl]amino}-1,2-dihydro-2,7-naphthyridin-1-one; 8-{[4-(1-cyclopropylpiperidin-4-yl)-3-methylphenyl]amino}-6-[(Z)-2-ethoxyethenyl]-1,2-dihydro-2,7-naphthyridin-1-one; 6-(3,6-dihydro-2H-thiopyran-4-yl)-8-{[4-(morpholin-4-yl)phenyl]amino}-1,2-dihydro-2,7-naphthyridin-1-one; 8-({3-methyl-4-[(2R)-2-methylmorpholin-4-yl]phenyl}amino)-6-propyl-1,2-dihydro-2,7-naphthyridin-1-one; 8-{[4-(morpholin-4-yl)phenyl]amino}-6-(thian-4-yl)-1,2-dihydro-2,7-naphthyridin-1-one; 8-{[4-(1-cyclopropylpiperidin-4-yl)-3-methylphenyl]amino}-6-(2-ethoxyethyl)-1,2-dihydro-2,7-naphthyridin-1-one; 8-{[4-(1-cyclopropylpiperidin-4-yl)-3-methylphenyl]amino}-6-[(1E)-prop-1-en-1-yl]-1,2-dihydro-2,7-naphthyridin-1-one; 8-{[4-(1-cyclopropylpiperidin-4-yl)-3-methylphenyl]amino}-6-(prop-1-en-2-yl)-1,2-dihydro-2,7-naphthyridin-1-one; 2-[4-(4-{[3-(6-methoxypyrazin-2-yl)-8-oxo-7,8-dihydro-2,7-naphthyridin-1-yl]amino}-2-methylphenyl)piperidin-1-yl]propanoic acid; methyl 2-[4-(4-{[3-(6-methoxypyrazin-2-yl)-8-oxo-7,8-dihydro-2,7-naphthyridin-1-yl]amino}-2-methylphenyl)piperidin-1-yl]butanoate; 2-[4-(4-{[3-(6-methoxypyrazin-2-yl)-8-oxo-7,8-dihydro-2,7-naphthyridin-1-yl]amino}-2-methylphenyl)piperidin-1-yl]butanoic acid; N-ethyl-2-[4-(4-{[3-(6-methoxypyrazin-2-yl)-8-oxo-7,8-dihydro-2,7-naphthyridin-1-yl]amino}-2-methylphenyl)piperidin-1-yl]propanamide; N-cyclopropyl-2-[4-(4-{[3-(6-methoxypyrazin-2-yl)-8-oxo-7,8-dihydro-2,7-naphthyridin-1-yl]amino}-2-methylphenyl)piperidin-1-yl]propanamide; N-(2-hydroxyethyl)-2-[4-(4-{[3-(6-methoxypyrazin-2-yl)-8-oxo-7,8-dihydro-2,7-naphthyridin-1-yl]amino}-2-methylphenyl)piperidin-1-yl]propanamide; N-(2-methoxyethyl)-2-[4-(4-{[3-(6-methoxypyrazin-2-yl)-8-oxo-7,8-dihydro-2,7-naphthyridin-1-yl]amino}-2-methylphenyl)piperidin-1-yl]propanamide; 2-[4-(4-{[3-(6-methoxypyrazin-2-yl)-8-oxo-7,8-dihydro-2,7-naphthyridin-1-yl]amino}-2-methylphenyl)piperidin-1-yl]-N-methylbutanamide; 2-methyl-6,8-bis[(4-methylpyridin-2-yl)amino]-1,2-dihydro-2,7-naphthyridin-1-one; 8-(cyclopropylamino)-6-(pyridin-2-ylamino)-1,2-dihydro-2,7-naphthyridin-1-one; 8-(cyclopropylamino)-6-[(4-methylpyridin-2-yl)amino]-1,2-dihydro-2,7-naphthyridin-1-one; 6-[(4-chloropyridin-2-yl)amino]-8-(cyclopropylamino)-1,2-dihydro-2,7-naphthyridin-1-one; 8-(cyclopropylamino)-6-[(4-methoxypyridin-2-yl)amino]-1,2-dihydro-2,7-naphthyridin-1-one; 2-methyl-6-[(4-methylpyridin-2-yl)amino]-8-(propan-2-ylamino)-1,2-dihydro-2,7-naphthyridin-1-one; 8-{[4-(1-{[1-(methoxymethyl)cyclopropyl]carbonyl}piperidin-4-yl)-3-methylphenyl]amino}-6-(6-methoxypyrazin-2-yl)-1,2-dihydro-2,7-naphthyridin-1-one; 8-({6-[(2R,6S)-2,6-dimethylmorpholin-4-yl]pyridin-3-yl}amino)-6-(2-methylpyrimidin-5-yl)-1,2-dihydro-2,7-naphthyridin-1-one; 8-({6-[(2R,6S)-2,6-dimethylmorpholin-4-yl]pyridin-3-yl}amino)-6-(pyrimidin-5-yl)-1,2-dihydro-2,7-naphthyridin-1-one; 6-(5-amino-6-methylpyrazin-2-yl)-8-({6-[(2R,6S)-2,6-dimethylmorpholin-4-yl]pyridin-3-yl}amino)-1,2-dihydro-2,7-naphthyridin-1-one; 8-{[4-(4-methoxypiperidin-1-yl)phenyl]amino}-6-(6-methoxypyrazin-2-yl)-1,2-dihydro-2,7-naphthyridin-1-one; 8-({3-methyl-4-[(2R)-2-methylmorpholin-4-yl]phenyl}amino)-6-(propan-2-yl)-1,2-dihydro-2,7-naphthyridin-1-one; 8-{[4-(1-cyclopropylpiperidin-4-yl)-3-methylphenyl]amino}-6-propyl-1,2-dihydro-2,7-naphthyridin-1-one; 8-{[4-(1-cyclopropylpiperidin-4-yl)-3-methylphenyl]amino}-6-(propan-2-yl)-1,2-dihydro-2,7-naphthyridin-1-one; 6-acetyl-8-{[3-methyl-4-(piperidin-4-yl)phenyl]amino}-1,2-dihydro-2,7-naphthyridin-1-one; 6-[(1E)-1-(methoxyimino)ethyl]-8-({3-methyl-4-[(2R)-2-methylmorpholin-4-yl]phenyl}amino)-1,2-dihydro-2,7-naphthyridin-1-one; 8-(butan-2-ylamino)-6-[(4-methylpyridin-2-yl)amino]-1,2-dihydro-2,7-naphthyridin-1-one; 8-(butan-2-ylamino)-6-(pyridin-2-ylamino)-1,2-dihydro-2,7-naphthyridin-1-one; 8-(butan-2-ylamino)-6-[(5-fluoropyridin-2-yl)amino]-1,2-dihydro-2,7-naphthyridin-1-one; 8-[(1-hydroxypropan-2-yl)amino]-6-[(4-methylpyridin-2-yl)amino]-1,2-dihydro-2,7-naphthyridin-1-one; 8-[(1-methoxypropan-2-yl)amino]-6-[(4-methylpyridin-2-yl)amino]-1,2-dihydro-2,7-naphthyridin-1-one; 8-{[(1R,2R)-2-hydroxycyclopentyl]amino}-6-[(4-methylpyridin-2-yl)amino]-1,2-dihydro-2,7-naphthyridin-1-one; 8-[(3-methylbutan-2-yl)amino]-6-[(4-methylpyridin-2-yl)amino]-1,2-dihydro-2,7-naphthyridin-1-one; 6-(1-ethoxyethyl)-8-({3-methyl-4-[(2R)-2-methylmorpholin-4-yl]phenyl}amino)-1,2-dihydro-2,7-naphthyridin-1-one; 6-[(1E)-1-(methoxyimino)ethyl]-8-{[3-methyl-4-(piperidin-4-yl)phenyl]amino}-1,2-dihydro-2,7-naphthyridin-1-one; 8-(cyclopropylamino)-6-{[4-(hydroxymethyl)pyridin-2-yl]amino}-1,2-dihydro-2,7-naphthyridin-1-one; 8-(cyclopropylamino)-6-[(4-ethylpyridin-2-yl)amino]-1,2-dihydro-2,7-naphthyridin-1-one; 8-(cyclopropylamino)-6-{[4-(trifluoromethyl)pyridin-2-yl]amino}-1,2-dihydro-2,7-naphthyridin-1-one; 2-{[1-(cyclopropylamino)-8-oxo-7,8-dihydro-2,7-naphthyridin-3-yl]amino}pyridine-4-carboxamide; 8-(cyclopropylamino)-2-methyl-6-[(4-methylpyridin-2-yl)amino]-1,2-dihydro-2,7-naphthyridin-1-one; 8-(ethylamino)-6-[(4-methylpyridin-2-yl)amino]-1,2-dihydro-2,7-naphthyridin-1-one; 8-[(2-hydroxyethyl)amino]-6-[(4-methylpyridin-2-yl)amino]-1,2-dihydro-2,7-naphthyridin-1-one; 8-[(2-methoxyethyl)amino]-6-[(4-methylpyridin-2-yl)amino]-1,2-dihydro-2,7-naphthyridin-1-one; 8-{[2-(dimethylamino)ethyl]amino}-6-[(4-methylpyridin-2-yl)amino]-1,2-dihydro-2,7-naphthyridin-1-one; 8-({[(2R)-1-ethylpyrrolidin-2-yl]methyl}amino)-6-[(4-methylpyridin-2-yl)amino]-1,2-dihydro-2,7-naphthyridin-1-one; 8-[(1-methylpiperidin-4-yl)amino]-6-[(4-methylpyridin-2-yl)amino]-1,2-dihydro-2,7-naphthyridin-1-one; 6-[(4-methylpyridin-2-yl)amino]-8-({[4-(morpholin-4-yl)phenyl]methyl}amino)-1,2-dihydro-2,7-naphthyridin-1-one; 6-[(4-methylpyridin-2-yl)amino]-8-{[(2R)-oxolan-2-ylmethyl]amino}-1,2-dihydro-2,7-naphthyridin-1-one; 8-(butylamino)-6-[(4-methylpyridin-2-yl)amino]-1,2-dihydro-2,7-naphthyridin-1-one; 8-[(4-methylpentan-2-yl)amino]-6-[(4-methylpyridin-2-yl)amino]-1,2-dihydro-2,7-naphthyridin-1-one; 6-[(4-methylpyridin-2-yl)amino]-8-(pyrrolidin-1-yl)-1,2-dihydro-2,7-naphthyridin-1-one; 8-{[(1S,2R)-2-hydroxycyclopentyl]amino}-6-[(4-methylpyridin-2-yl)amino]-1,2-dihydro-2,7-naphthyridin-1-one; 6-[(4-methylpyridin-2-yl)amino]-8-(morpholin-4-yl)-1,2-dihydro-2,7-naphthyridin-1-one; 6-[(4-methylpyridin-2-yl)amino]-8-(oxolan-3-ylamino)-1,2-dihydro-2,7-naphthyridin-1-one; 8-[(1-hydroxypropan-2-yl)amino]-6-(pyridin-2-ylamino)-1,2-dihydro-2,7-naphthyridin-1-one; 8-[(4-methylpentan-2-yl)amino]-6-(pyridin-2-ylamino)-1,2-dihydro-2,7-naphthyridin-1-one; 8-[(1-methoxypropan-2-yl)amino]-6-(pyridin-2-ylamino)-1,2-dihydro-2,7-naphthyridin-1-one; 8-{[(1R,2R)-2-hydroxycyclopentyl]amino}-6-(pyridin-2-ylamino)-1,2-dihydro-2,7-naphthyridin-1-one; 8-[(3-methylbutan-2-yl)amino]-6-(pyridin-2-ylamino)-1,2-dihydro-2,7-naphthyridin-1-one; 6-(pyridin-2-ylamino)-8-(pyrrolidin-1-yl)-1,2-dihydro-2,7-naphthyridin-1-one; 8-{[(1S,2R)-2-hydroxycyclopentyl]amino}-6-(pyridin-2-ylamino)-1,2-dihydro-2,7-naphthyridin-1-one; 8-(morpholin-4-yl)-6-(pyridin-2-ylamino)-1,2-dihydro-2,7-naphthyridin-1-one; 8-(oxolan-3-ylamino)-6-(pyridin-2-ylamino)-1,2-dihydro-2,7-naphthyridin-1-one; 6-(2-aminopyrimidin-5-yl)-8-[(1-methoxypropan-2-yl)amino]-1,2-dihydro-2,7-naphthyridin-1-one; 6-(2-aminopyrimidin-5-yl)-8-(pyrrolidin-1-yl)-1,2-dihydro-2,7-naphthyridin-1-one; 6-(2-aminopyrimidin-5-yl)-8-{[(1S,2R)-2-hydroxycyclopentyl]amino}-1,2-dihydro-2,7-naphthyridin-1-one; 8-(propan-2-ylamino)-6-[(4-propylpyridin-2-yl)amino]-1,2-dihydro-2,7-naphthyridin-1-one; 8-[(1-benzylpyrrolidin-3-yl)amino]-6-[(4-methylpyridin-2-yl)amino]-1,2-dihydro-2,7-naphthyridin-1-one; 6-[(4-methylpyridin-2-yl)amino]-8-{[(2S)-oxolan-2-ylmethyl]amino}-1,2-dihydro-2,7-naphthyridin-1-one; 8-(cyclobutylamino)-6-[(4-methylpyridin-2-yl)amino]-1,2-dihydro-2,7-naphthyridin-1-one; 8-{[2-(3-chlorophenyl)-2-hydroxyethyl]amino}-6-[(4-methylpyridin-2-yl)amino]-1,2-dihydro-2,7-naphthyridin-1-one; 8-{[(2R)-2-hydroxypropyl]amino}-6-[(4-methylpyridin-2-yl)amino]-1,2-dihydro-2,7-naphthyridin-1-one; 8-{[(2S)-2-hydroxypropyl]amino}-6-[(4-methylpyridin-2-yl)amino]-1,2-dihydro-2,7-naphthyridin-1-one; 6-(2-aminopyrimidin-5-yl)-8-[(2-hydroxyethyl)amino]-1,2-dihydro-2,7-naphthyridin-1-one; 6-(2-aminopyrimidin-5-yl)-8-[(2-methoxyethyl)amino]-1,2-dihydro-2,7-naphthyridin-1-one; 8-[(3-hydroxypropyl)amino]-6-[(4-methylpyridin-2-yl)amino]-1,2-dihydro-2,7-naphthyridin-1-one; 6-[(4-methylpyridin-2-yl)amino]-8-[(piperidin-4-ylmethyl)amino]-1,2-dihydro-2,7-naphthyridin-1-one; 8-({[(2S)-1-ethylpyrrolidin-2-yl]methyl}amino)-6-[(4-methylpyridin-2-yl)amino]-1,2-dihydro-2,7-naphthyridin-1-one; 8-(benzylamino)-6-[(4-methylpyridin-2-yl)amino]-1,2-dihydro-2,7-naphthyridin-1-one; 6-[(4-methylpyridin-2-yl)amino]-8-[(2,2,2-trifluoroethyl)amino]-1,2-dihydro-2,7-naphthyridin-1-one; 6-(2-aminopyrimidin-5-yl)-8-[(1-hydroxypropan-2-yl)amino]-1,2-dihydro-2,7-naphthyridin-1-one; 8-(propan-2-ylamino)-6-(pyrazin-2-ylamino)-1,2-dihydro-2,7-naphthyridin-1-one; 6-(2-aminopyrimidin-5-yl)-8-{[(1R,2R)-2-hydroxycyclopentyl]amino}-1,2-dihydro-2,7-naphthyridin-1-one; 6-[(5-chloropyridin-2-yl)amino]-8-(propan-2-ylamino)-1,2-dihydro-2,7-naphthyridin-1-one; 8-{[4-(4-oxocyclohexyl)phenyl]amino}-6-(pyrimidin-5-yl)-1,2-dihydro-2,7-naphthyridin-1-one; 6-{[8-oxo-1-(propan-2-ylamino)-7,8-dihydro-2,7-naphthyridin-3-yl]amino}pyridine-3-carbonitrile; 6-(2-aminopyrimidin-5-yl)-8-(oxolan-3-ylamino)-1,2-dihydro-2,7-naphthyridin-1-one; 6-{[8-oxo-1-(propan-2-ylamino)-7,8-dihydro-2,7-naphthyridin-3-yl]amino}pyridine-3-carboxamide; 8-[(3-methoxypropyl)amino]-6-[(4-methylpyridin-2-yl)amino]-1,2-dihydro-2,7-naphthyridin-1-one; 8-[(2-methylpropyl)amino]-6-[(4-methylpyridin-2-yl)amino]-1,2-dihydro-2,7-naphthyridin-1-one; 8-({4-[4-(dimethylamino)cyclohexyl]phenyl}amino)-6-(pyrimidin-5-yl)-1,2-dihydro-2,7-naphthyridin-1-one; 8-[(2S)-butan-2-ylamino]-6-[(4-methylpyridin-2-yl)amino]-1,2-dihydro-2,7-naphthyridin-1-one; 6-[(4-methylpyridin-2-yl)amino]-8-{[2-(pyridin-3-yl)ethyl]amino}-1,2-dihydro-2,7-naphthyridin-1-one; 8-[(cyclopropylmethyl)amino]-6-[(4-methylpyridin-2-yl)amino]-1,2-dihydro-2,7-naphthyridin-1-one; 6-(2-aminopyrimidin-5-yl)-8-[(2-methylpropyl)amino]-1,2-dihydro-2,7-naphthyridin-1-one; 8-[(4-{4-[(2R)-2-methylmorpholin-4-yl]cyclohexyl}phenyl)amino]-6-(pyrimidin-5-yl)-1,2-dihydro-2,7-naphthyridin-1-one; 6-(2-aminopyrimidin-5-yl)-8-[(2S)-butan-2-ylamino]-1,2-dihydro-2,7-naphthyridin-1-one; 6-(2-aminopyrimidin-5-yl)-8-{[2-(pyridin-3-yl)ethyl]amino}-1,2-dihydro-2,7-naphthyridin-1-one; 8-{[4-(1-tert-butyl-1,2,3,6-tetrahydropyridin-4-yl)phenyl]amino}-6-(pyrimidin-5-yl)-1,2-dihydro-2,7-naphthyridin-1-one; 6-(2-aminopyrimidin-5-yl)-8-[(cyclopropylmethyl)amino]-1,2-dihydro-2,7-naphthyridin-1-one; 8-[(3-methoxypropyl)amino]-6-(pyridin-2-ylamino)-1,2-dihydro-2,7-naphthyridin-1-one; 8-[(2-methylpropyl)amino]-6-(pyridin-2-ylamino)-1,2-dihydro-2,7-naphthyridin-1-one; 6-(pyridin-2-ylamino)-8-{[2-(pyridin-3-yl)ethyl]amino}-1,2-dihydro-2,7-naphthyridin-1-one; 6-(6-amino-5-methylpyridin-3-yl)-8-({4-[4-(morpholin-4-yl)cyclohexyl]phenyl}amino)-1,2-dihydro-2,7-naphthyridin-1-one; 6-(5-amino-6-methylpyrazin-2-yl)-8-({4-[4-(morpholin-4-yl)cyclohexyl]phenyl}amino)-1,2-dihydro-2,7-naphthyridin-1-one; 6-(1-methyl-1H-pyrazol-4-yl)-8-({4-[4-(morpholin-4-yl)cyclohexyl]phenyl}amino)-1,2-dihydro-2,7-naphthyridin-1-one; 6-(2-amino-4-methylpyrimidin-5-yl)-8-({4-[4-(morpholin-4-yl)cyclohexyl]phenyl}amino)-1,2-dihydro-2,7-naphthyridin-1-one; 6-(6-methoxypyrazin-2-yl)-8-{[10-(2,2,2-trifluoroacetyl)-10-azatricyclo[6.3.1.0̂{2,7}]dodeca-2,4,6-trien-4-yl]amino}-1,2-dihydro-2,7-naphthyridin-1-one; 8-{10-azatricyclo[6.3.1.0̂{2,7}]dodeca-2,4,6-trien-4-ylamino}-6-(6-methoxypyrazin-2-yl)-1,2-dihydro-2,7-naphthyridin-1-one; 6-(pyrimidin-5-yl)-8-{[10-(2,2,2-trifluoroacetyl)-10-azatricyclo[6.3.1.0̂{2,7}]dodeca-2,4,6-trien-4-yl]amino}-1,2-dihydro-2,7-naphthyridin-1-one; 8-{10-azatricyclo[6.3.1.0̂{2,7}]dodeca-2,4,6-trien-4-ylamino}-6-(pyrimidin-5-yl)-1,2-dihydro-2,7-naphthyridin-1-one; 8-{[4-(1-cyclopropylpiperidin-4-yl)-3-methylphenyl]amino}-6-(3-methoxypropyl)-1,2-dihydro-2,7-naphthyridin-1-one; 6-(3-methoxypropyl)-8-(3-methyl-4-{[(2R)-2-methylmorpholin-4-yl]phenyl}amino)-1,2-dihydro-2,7-naphthyridin-1-one; 6-(3-methoxypropyl)-8-{[4-(morpholin-4-yl)phenyl]amino}-1,2-dihydro-2,7-naphthyridin-1-one; (2E)-3-[1-({6-[(2R)-2-methylmorpholin-4-yl]pyridin-3-yl}amino)-8-oxo-7,8-dihydro-2,7-naphthyridin-3-yl]prop-2-enoic acid; 8-[(2S)-butan-2-ylamino]-6-(pyridin-2-ylamino)-1,2-dihydro-2,7-naphthyridin-1-one; 8-[(cyclopropylmethyl)amino]-6-(pyridin-2-ylamino)-1,2-dihydro-2,7-naphthyridin-1-one; 8-(cyclopentylamino)-6-[(4-methylpyridin-2-yl)amino]-1,2-dihydro-2,7-naphthyridin-1-one; 6-[(4-methylpyridin-2-yl)amino]-8-(oxan-4-ylamino)-1,2-dihydro-2,7-naphthyridin-1-one; 8-{[(2S)-1-hydroxypropan-2-yl]amino}-6-[(4-methylpyridin-2-yl)amino]-1,2-dihydro-2,7-naphthyridin-1-one; 8-{[(2R)-1-hydroxypropan-2-yl]amino}-6-[(4-methylpyridin-2-yl)amino]-1,2-dihydro-2,7-naphthyridin-1-one; 8-(cyclopentylamino)-6-(pyridin-2-ylamino)-1,2-dihydro-2,7-naphthyridin-1-one; 8-(oxan-4-ylamino)-6-(pyridin-2-ylamino)-1,2-dihydro-2,7-naphthyridin-1-one; 8-{[(2S)-1-hydroxypropan-2-yl]amino}-6-(pyridin-2-ylamino)-1,2-dihydro-2,7-naphthyridin-1-one; 8-{[(2R)-1-hydroxypropan-2-yl]amino}-6-(pyridin-2-ylamino)-1,2-dihydro-2,7-naphthyridin-1-one; 8-({4-[4-(4-methylpiperazin-1-yl)cyclohexyl]phenyl}amino)-6-(pyrimidin-5-yl)-1,2-dihydro-2,7-naphthyridin-1-one; 6-(6-methoxypyrazin-2-yl)-8-[(3-methyl-4-{1-[(oxan-4-yl)carbonyl]piperidin-4-yl}phenyl)amino]-1,2-dihydro-2,7-naphthyridin-1-one; 6-[(4-methylpyridin-2-yl)amino]-8-{[2-(1-methylpyrrolidin-2-yl)ethyl]amino}-1,2-dihydro-2,7-naphthyridin-1-one; 8-{[(2S)-1-methoxypropan-2-yl]amino}-6-[(4-methylpyridin-2-yl)amino]-1,2-dihydro-2,7-naphthyridin-1-one; 8-(tert-butylamino)-6-[(4-methylpyridin-2-yl)amino]-1,2-dihydro-2,7-naphthyridin-1-one; 4-fluoro-2-methyl-6-[(4-methylpyridin-2-yl)amino]-8-(propan-2-ylamino)-1,2-dihydro-2,7-naphthyridin-1-one; ethyl (2E)-3-(1-{[4-(morpholin-4-yl)phenyl]amino}-8-oxo-7,8-dihydro-2,7-naphthyridin-3-yl)prop-2-enoate; 6-(2-ethoxyethyl)-8-{[4-(morpholin-4-yl)phenyl]amino}-1,2-dihydro-2,7-naphthyridin-1-one; 6-(2-hydroxyethyl)-8-{[4-(morpholin-4-yl)phenyl]amino}-1,2-dihydro-2,7-naphthyridin-1-one; ethyl 3-(1-{[4-(morpholin-4-yl)phenyl]amino}-8-oxo-7,8-dihydro-2,7-naphthyridin-3-yl)propanoate; 8-{[4-(1-cyclopropylpiperidin-4-yl)-3-methylphenyl]amino}-6-(2-hydroxyethyl)-1,2-dihydro-2,7-naphthyridin-1-one; 6-{[4-(hydroxymethyl)pyridin-2-yl]amino}-8-(propan-2-ylamino)-1,2-dihydro-2,7-naphthyridin-1-one; 2-{[8-oxo-1-(propan-2-ylamino)-7,8-dihydro-2,7-naphthyridin-3-yl]amino}pyridine-4-carboxamide; 8-[(2-methylbutan-2-yl)amino]-6-[(4-methylpyridin-2-yl)amino]-1,2-dihydro-2,7-naphthyridin-1-one; 8-[(1-hydroxy-2-methylpropan-2-yl)amino]-6-[(4-methylpyridin-2-yl)amino]-1,2-dihydro-2,7-naphthyridin-1-one; 6-(3-hydroxypropyl)-8-{[4-(morpholin-4-yl)phenyl]amino}-1,2-dihydro-2,7-naphthyridin-1-one; 8-(azetidin-3-ylamino)-6-[(4-methylpyridin-2-yl)amino]-1,2-dihydro-2,7-naphthyridin-1-one; 6-[(4-methylpyridin-2-yl)amino]-8-[(3R)-oxolan-3-ylamino]-1,2-dihydro-2,7-naphthyridin-1-one; 6-[(4-methylpyridin-2-yl)amino]-8-[(3S)-oxolan-3-ylamino]-1,2-dihydro-2,7-naphthyridin-1-one; 8-[(4-hydroxycyclohexyl)amino]-6-[(4-methylpyridin-2-yl)amino]-1,2-dihydro-2,7-naphthyridin-1-one; 6-(2-aminopyrimidin-5-yl)-8-{[(2S)-1-methoxypropan-2-yl]amino}-1,2-dihydro-2,7-naphthyridin-1-one; 6-(2-aminopyrimidin-5-yl)-8-{[(2S)-2-(3-chlorophenyl)-2-hydroxyethyl]amino}-1,2-dihydro-2,7-naphthyridin-1-one; 6-(2-aminopyrimidin-5-yl)-8-[(2,2,2-trifluoroethyl)amino]-1,2-dihydro-2,7-naphthyridin-1-one; 6-(2-aminopyrimidin-5-yl)-8-{[(2S)-2-hydroxypropyl]amino}-1,2-dihydro-2,7-naphthyridin-1-one; 6-(2-aminopyrimidin-5-yl)-8-{[(2R)-2-hydroxypropyl]amino}-1,2-dihydro-2,7-naphthyridin-1-one; 8-{[(2S)-2-(3-chlorophenyl)-2-hydroxyethyl]amino}-6-[(4-methylpyridin-2-yl)amino]-1,2-dihydro-2,7-naphthyridin-1-one; 8-[(3R)-3-hydroxypyrrolidin-1-yl]-6-[(4-methylpyridin-2-yl)amino]-1,2-dihydro-2,7-naphthyridin-1-one; 8-[(3S)-3-(hydroxymethyl)pyrrolidin-1-yl]-6-[(4-methylpyridin-2-yl)amino]-1,2-dihydro-2,7-naphthyridin-1-one; 8-[(3R)-3-(hydroxymethyl)pyrrolidin-1-yl]-6-[(4-methylpyridin-2-yl)amino]-1,2-dihydro-2,7-naphthyridin-1-one; 6-(2-aminopyrimidin-5-yl)-8-(cyclobutylamino)-1,2-dihydro-2,7-naphthyridin-1-one; 6-(2-aminopyrimidin-5-yl)-8-[(2-methylbutan-2-yl)amino]-1,2-dihydro-2,7-naphthyridin-1-one; 6-(2-aminopyrimidin-5-yl)-8-(tert-butylamino)-1,2-dihydro-2,7-naphthyridin-1-one; 2-ethyl-6-[(4-methylpyridin-2-yl)amino]-8-(propan-2-ylamino)-1,2-dihydro-2,7-naphthyridin-1-one; 2-ethyl-6-[(4-ethylpyridin-2-yl)amino]-8-(propan-2-ylamino)-1,2-dihydro-2,7-naphthyridin-1-one; 6-[(4-chloropyridin-2-yl)amino]-2-ethyl-8-(propan-2-ylamino)-1,2-dihydro-2,7-naphthyridin-1-one; 2-ethyl-8-(propan-2-ylamino)-6-[(4-propylpyridin-2-yl)amino]-1,2-dihydro-2,7-naphthyridin-1-one; 2-ethyl-8-(propan-2-ylamino)-6-(pyrazin-2-ylamino)-1,2-dihydro-2,7-naphthyridin-1-one; 2-methyl-6-[(4-methylpyridin-2-yl)amino]-8-{[4-(piperidin-4-yl)phenyl]amino}-1,2-dihydro-2,7-naphthyridin-1-one; 2-methyl-8-{[4-(1-methylpiperidin-4-yl)phenyl]amino}-6-[(4-methylpyridin-2-yl)amino]-1,2-dihydro-2,7-naphthyridin-1-one; 8-{[4-(1-acetylpiperidin-4-yl)phenyl]amino}-2-methyl-6-[(4-methylpyridin-2-yl)amino]-1,2-dihydro-2,7-naphthyridin-1-one; 8-({4-[1-(2-methanesulfonylethyl)piperidin-4-yl]phenyl}amino)-2-methyl-6-[(4-methylpyridin-2-yl)amino]-1,2-dihydro-2,7-naphthyridin-1-one; 3-{4-[4-({7-methyl-3-[(4-methylpyridin-2-yl)amino]-8-oxo-7,8-dihydro-2,7-naphthyridin-1-yl}amino)phenyl]piperidin-1-yl}propanenitrile; 2-(2-hydroxyethyl)-6-[(4-methylpyridin-2-yl)amino]-8-(propan-2-ylamino)-1,2-dihydro-2,7-naphthyridin-1-one; 2-(2-methoxyethyl)-6-[(4-methylpyridin-2-yl)amino]-8-(propan-2-ylamino)-1,2-dihydro-2,7-naphthyridin-1-one; 6-[(4-methylpyridin-2-yl)amino]-2-[2-(morpholin-4-yl)ethyl]-8-(propan-2-ylamino)-1,2-dihydro-2,7-naphthyridin-1-one; N-ethyl-2-{6-[(4-methylpyridin-2-yl)amino]-1-oxo-8-(propan-2-ylamino)-1,2-dihydro-2,7-naphthyridin-2-yl}acetamide; 2-(2-hydroxyethyl)-8-(propan-2-ylamino)-6-(pyrazin-2-ylamino)-1,2-dihydro-2,7-naphthyridin-1-one; 2-(2-methoxyethyl)-8-(propan-2-ylamino)-6-(pyrazin-2-ylamino)-1,2-dihydro-2,7-naphthyridin-1-one; 2-[2-(morpholin-4-yl)ethyl]-8-(propan-2-ylamino)-6-(pyrazin-2-ylamino)-1,2-dihydro-2,7-naphthyridin-1-one; N-ethyl-2-[1-oxo-8-(propan-2-ylamino)-6-(pyrazin-2-ylamino)-1,2-dihydro-2,7-naphthyridin-2-yl]acetamide; 8-{[(2S)-1-hydroxypropan-2-yl]amino}-2-methyl-6-[(4-methylpyridin-2-yl)amino]-1,2-dihydro-2,7-naphthyridin-1-one; 8-{[(1S,2R)-2-hydroxycyclopentyl]amino}-2-methyl-6-[(4-methylpyridin-2-yl)amino]-1,2-dihydro-2,7-naphthyridin-1-one; 2-methyl-6-[(4-methylpyridin-2-yl)amino]-8-[(3S)-oxolan-3-ylamino]-1,2-dihydro-2,7-naphthyridin-1-one; 8-[(4-hydroxycyclohexyl)amino]-2-methyl-6-[(4-methylpyridin-2-yl)amino]-1,2-dihydro-2,7-naphthyridin-1-one; 8-{[(2S)-1-hydroxypropan-2-yl]amino}-2-methyl-6-(pyrazin-2-ylamino)-1,2-dihydro-2,7-naphthyridin-1-one; 8-{[(1S,2R)-2-hydroxycyclopentyl]amino}-2-methyl-6-(pyrazin-2-ylamino)-1,2-dihydro-2,7-naphthyridin-1-one; 2-methyl-8-[(3S)-oxolan-3-ylamino]-6-(pyrazin-2-ylamino)-1,2-dihydro-2,7-naphthyridin-1-one; 8-[(4-hydroxycyclohexyl)amino]-2-methyl-6-(pyrazin-2-ylamino)-1,2-dihydro-2,7-naphthyridin-1-one; 8-[(3-hydroxypropyl)amino]-2-methyl-6-[(4-methylpyridin-2-yl)amino]-1,2-dihydro-2,7-naphthyridin-1-one; 2-methyl-6-[(4-methylpyridin-2-yl)amino]-8-[(2,2,2-trifluoroethyl)amino]-1,2-dihydro-2,7-naphthyridin-1-one; 8-(tert-butylamino)-2-methyl-6-[(4-methylpyridin-2-yl)amino]-1,2-dihydro-2,7-naphthyridin-1-one; 8-[(1-hydroxy-2-methylpropan-2-yl)amino]-2-methyl-6-[(4-methylpyridin-2-yl)amino]-1,2-dihydro-2,7-naphthyridin-1-one; 8-(cyclobutylamino)-2-methyl-6-[(4-methylpyridin-2-yl)amino]-1,2-dihydro-2,7-naphthyridin-1-one; 2-methyl-6-[(4-methylpyridin-2-yl)amino]-8-{[(2R)-oxolan-2-ylmethyl]amino}-1,2-dihydro-2,7-naphthyridin-1-one; 8-{[(2R)-2-hydroxypropyl]amino}-2-methyl-6-[(4-methylpyridin-2-yl)amino]-1,2-dihydro-2,7-naphthyridin-1-one; 2-methyl-6-[(4-methylpyridin-2-yl)amino]-8-({4-[(morpholin-4-yl)carbonyl]phenyl}amino)-1,2-dihydro-2,7-naphthyridin-1-one; N-[2-(diethylamino)ethyl]-4-({7-methyl-3-[(4-methylpyridin-2-yl)amino]-8-oxo-7,8-dihydro-2,7-naphthyridin-1-yl}amino)benzamide; 8-[(4-methanesulfonylphenyl)amino]-2-methyl-6-[(4-methylpyridin-2-yl)amino]-1,2-dihydro-2,7-naphthyridin-1-one; 3-({7-methyl-3-[(4-methylpyridin-2-yl)amino]-8-oxo-7,8-dihydro-2,7-naphthyridin-1-yl}amino)benzene-1-sulfonamide; 8-{[4-(2-ethoxyethoxy)phenyl]amino}-2-methyl-6-[(4-methylpyridin-2-yl)amino]-1,2-dihydro-2,7-naphthyridin-1-one; 8-{[3-(2-ethoxyethoxy)phenyl]amino}-2-methyl-6-[(4-methylpyridin-2-yl)amino]-1,2-dihydro-2,7-naphthyridin-1-one; 8-[(4-methanesulfonylphenyl)amino]-2-methyl-6-(pyrazin-2-ylamino)-1,2-dihydro-2,7-naphthyridin-1-one; 8-{[3-(2-ethoxyethoxy)phenyl]amino}-2-methyl-6-(pyrazin-2-ylamino)-1,2-dihydro-2,7-naphthyridin-1-one; 8-({4-[4-(dimethylamino)cyclohexyl]phenyl}amino)-6-(2-methylpyrimidin-5-yl)-1,2-dihydro-2,7-naphthyridin-1-one; 3-[4-(2-methyl-4-{[3-(2-methylpyrimidin-5-yl)-8-oxo-7,8-dihydro-2,7-naphthyridin-1-yl]amino}-phenyl)piperidin-1-yl]-1$1̂{6}-thietane-1,1-dione; 8-({4-[4-(4-ethylpiperazin-1-yl)cyclohexyl]phenyl}amino)-6-(pyrimidin-5-yl)-1,2-dihydro-2,7-naphthyridin-1-one; 8-({4-[4-(4-methylpiperazin-1-yl)cyclohexyl]phenyl}amino)-6-(pyrimidin-5-yl)-1,2-dihydro-2,7-naphthyridin-1-one; 8-({4-[4-(dimethylamino)cyclohexyl]phenyl}amino)-6-(6-methoxypyrazin-2-yl)-1,2-dihydro-2,7-naphthyridin-1-one; N-[2-(diethylamino)ethyl]-4-{[7-methyl-8-oxo-3-(pyrazin-2-ylamino)-7,8-dihydro-2,7-naphthyridin-1-yl]amino}benzamide; 3-{[7-methyl-8-oxo-3-(pyrazin-2-ylamino)-7,8-dihydro-2,7-naphthyridin-1-yl]amino}benzene-1-sulfonamide; 8-{[4-(2-ethoxyethoxy)phenyl]amino}-2-methyl-6-(pyrazin-2-ylamino)-1,2-dihydro-2,7-naphthyridin-1-one; 2-methyl-8-{[4-(piperidin-4-yl)phenyl]amino}-6-(pyrazin-2-ylamino)-1,2-dihydro-2,7-naphthyridin-1-one; N-methyl-4-({7-methyl-3-[(4-methylpyridin-2-yl)amino]-8-oxo-7,8-dihydro-2,7-naphthyridin-1-yl}amino)benzamide; N-methyl-4-{[7-methyl-8-oxo-3-(pyrazin-2-ylamino)-7,8-dihydro-2,7-naphthyridin-1-yl]amino}benzamide; 2-methyl-8-{[4-(1-methylpiperidin-4-yl)phenyl]amino}-6-(pyrazin-2-ylamino)-1,2-dihydro-2,7-naphthyridin-1-one; 8-{[4-(1-acetylpiperidin-4-yl)phenyl]amino}-2-methyl-6-(pyrazin-2-ylamino)-1,2-dihydro-2,7-naphthyridin-1-one; 2-methyl-8-({4-[1-(oxetan-3-yl)piperidin-4-yl]phenyl}amino)-6-(pyrazin-2-ylamino)-1,2-dihydro-2,7-naphthyridin-1-one; 2-methyl-8-(propan-2-ylamino)-6-(pyrazin-2-ylamino)-1,2-dihydro-2,7-naphthyridin-1-one; 2-ethyl-6-[(3-methylpyrazin-2-yl)amino]-8-(propan-2-ylamino)-1,2-dihydro-2,7-naphthyridin-1-one; 8-[(3-hydroxypropyl)amino]-2-methyl-6-(pyrazin-2-ylamino)-1,2-dihydro-2,7-naphthyridin-1-one; 2-methyl-6-(pyrazin-2-ylamino)-8-[(2,2,2-trifluoroethyl)amino]-1,2-dihydro-2,7-naphthyridin-1-one; 8-(tert-butylamino)-2-methyl-6-(pyrazin-2-ylamino)-1,2-dihydro-2,7-naphthyridin-1-one; 8-[(1-hydroxy-2-methylpropan-2-yl)amino]-2-methyl-6-(pyrazin-2-ylamino)-1,2-dihydro-2,7-naphthyridin-1-one; 8-(cyclobutylamino)-2-methyl-6-(pyrazin-2-ylamino)-1,2-dihydro-2,7-naphthyridin-1-one; 8-(tert-butylamino)-2-(2-hydroxyethyl)-6-[(4-methylpyridin-2-yl)amino]-1,2-dihydro-2,7-naphthyridin-1-one; 8-(tert-butylamino)-2-(2-hydroxyethyl)-6-(pyrazin-2-ylamino)-1,2-dihydro-2,7-naphthyridin-1-one; 8-[(1-hydroxy-2-methylpropan-2-yl)amino]-2-(2-hydroxyethyl)-6-[(4-methylpyridin-2-yl)amino]-1,2-dihydro-2,7-naphthyridin-1-one; 8-[(1-hydroxy-2-methylpropan-2-yl)amino]-2-(2-hydroxyethyl)-6-(pyrazin-2-ylamino)-1,2-dihydro-2,7-naphthyridin-1-one; N-methyl-4-({7-methyl-3-[(4-methylpyridin-2-yl)amino]-8-oxo-7,8-dihydro-2,7-naphthyridin-1-yl}amino)benzene-1-sulfonamide; N-methyl-4-{[7-methyl-8-oxo-3-(pyrazin-2-ylamino)-7,8-dihydro-2,7-naphthyridin-1-yl]amino}benzene-1-sulfonamide; 8-[(4-methanesulfonylphenyl)amino]-2-methyl-6-[(4-methylpyridin-2-yl)amino]-1,2-dihydro-2,7-naphthyridin-1-one; 2-ethyl-8-{[(2S)-1-hydroxypropan-2-yl]amino}-6-(pyrazin-2-ylamino)-1,2-dihydro-2,7-naphthyridin-1-one; 2-ethyl-8-{[(1S,2R)-2-hydroxycyclopentyl]amino}-6-(pyrazin-2-ylamino)-1,2-dihydro-2,7-naphthyridin-1-one; 2-ethyl-8-[(3S)-oxolan-3-ylamino]-6-(pyrazin-2-ylamino)-1,2-dihydro-2,7-naphthyridin-1-one; 2-ethyl-8-[(4-hydroxycyclohexyl)amino]-6-(pyrazin-2-ylamino)-1,2-dihydro-2,7-naphthyridin-1-one; 8-[(4-hydroxycyclohexyl)amino]-2-methyl-6-[(4-methylpyridin-2-yl)amino]-1,2-dihydro-2,7-naphthyridin-1-one; 8-[(4-hydroxycyclohexyl)amino]-2-methyl-6-(pyrazin-2-ylamino)-1,2-dihydro-2,7-naphthyridin-1-one; 2-ethyl-8-{[(2S)-1-hydroxypropan-2-yl]amino}-6-[(4-methylpyridin-2-yl)amino]-1,2-dihydro-2,7-naphthyridin-1-one; 2-ethyl-8-{[(1S,2R)-2-hydroxycyclopentyl]amino}-6-[(4-methylpyridin-2-yl)amino]-1,2-dihydro-2,7-naphthyridin-1-one; 2-ethyl-6-[(4-methylpyridin-2-yl)amino]-8-[(3S)-oxolan-3-ylamino]-1,2-dihydro-2,7-naphthyridin-1-one; 2-ethyl-8-[(4-hydroxycyclohexyl)amino]-6-[(4-methylpyridin-2-yl)amino]-1,2-dihydro-2,7-naphthyridin-1-one; 4-({7-methyl-3-[(4-methylpyridin-2-yl)amino]-8-oxo-7,8-dihydro-2,7-naphthyridin-1-yl}amino)-N-propylbenz amide; 8-[(4-methoxyphenyl)amino]-2-methyl-6-[(4-methylpyridin-2-yl)amino]-1,2-dihydro-2,7-naphthyridin-1-one; 8-[(4-methoxyphenyl)amino]-2-methyl-6-(pyrazin-2-ylamino)-1,2-dihydro-2,7-naphthyridin-1-one; 8-(tert-butylamino)-2-ethyl-6-[(4-methylpyridin-2-yl)amino]-1,2-dihydro-2,7-naphthyridin-1-one; 8-(tert-butylamino)-2-ethyl-6-(pyrazin-2-ylamino)-1,2-dihydro-2,7-naphthyridin-1-one; 2-ethyl-8-[(1-hydroxy-2-methylpropan-2-yl)amino]-6-[(4-methylpyridin-2-yl)amino]-1,2-dihydro-2,7-naphthyridin-1-one; 2-ethyl-8-[(1-hydroxy-2-methylpropan-2-yl)amino]-6-(pyrazin-2-ylamino)-1,2-dihydro-2,7-naphthyridin-1-one; 3-({7-methyl-3-[(4-methylpyridin-2-yl)amino]-8-oxo-7,8-dihydro-2,7-naphthyridin-1-yl}amino)benzamide; 8-[(1-hydroxy-2-methylpropan-2-yl)amino]-2-methyl-6-[(4-methylpyridin-2-yl)amino]-1,2-dihydro-2,7-naphthyridin-1-one; 8-(tert-butylamino)-2-methyl-6-(pyrazin-2-ylamino)-1,2-dihydro-2,7-naphthyridin-1-one; 4-{[7-methyl-8-oxo-3-(pyrazin-2-ylamino)-7,8-dihydro-2,7-naphthyridin-1-yl]amino}-N-propylbenzamide; 2-(2-hydroxyethyl)-8-{[(2S)-1-hydroxypropan-2-yl]amino}-6-[(4-methylpyridin-2-yl)amino]-1,2-dihydro-2,7-naphthyridin-1-one; 2-(2-hydroxyethyl)-6-[(4-methylpyridin-2-yl)amino]-8-[(3S)-oxolan-3-ylamino]-1,2-dihydro-2,7-naphthyridin-1-one; 8-{[(1S,2R)-2-hydroxycyclopentyl]amino}-2-(2-hydroxyethyl)-6-[(4-methylpyridin-2-yl)amino]-1,2-dihydro-2,7-naphthyridin-1-one; 8-[(4-hydroxycyclohexyl)amino]-2-(2-hydroxyethyl)-6-[(4-methylpyridin-2-yl)amino]-1,2-dihydro-2,7-naphthyridin-1-one; 8-[(3,4-dimethoxyphenyl)amino]-6-[(4-methylpyridin-2-yl)amino]-1,2-dihydro-2,7-naphthyridin-1-one; 2-methyl-6-[(4-methylpyridin-2-yl)amino]-8-{[6-(oxan-4-yl)pyridin-3-yl]amino}-1,2-dihydro-2,7-naphthyridin-1-one; 2-(2-hydroxyethyl)-8-{[(2S)-1-hydroxypropan-2-yl]amino}-6-(pyrazin-2-ylamino)-1,2-dihydro-2,7-naphthyridin-1-one; 8-{[(1S,2R)-2-hydroxycyclopentyl]amino}-2-(2-hydroxyethyl)-6-(pyrazin-2-ylamino)-1,2-dihydro-2,7-naphthyridin-1-one; 2-(2-hydroxyethyl)-8-[(3S)-oxolan-3-ylamino]-6-(pyrazin-2-ylamino)-1,2-dihydro-2,7-naphthyridin-1-one; 8-[(4-hydroxycyclohexyl)amino]-2-(2-hydroxyethyl)-6-(pyrazin-2-ylamino)-1,2-dihydro-2,7-naphthyridin-1-one; 8-[(4-hydroxycyclohexyl)amino]-2-methyl-6-(pyrazin-2-ylamino)-1,2-dihydro-2,7-naphthyridin-1-one; 8-[(4-hydroxycyclohexyl)amino]-2-methyl-6-[(4-methylpyridin-2-yl)amino]-1,2-dihydro-2,7-naphthyridin-1-one; 8-{[(2R)-2-hydroxypropyl]amino}-2-methyl-6-(pyrazin-2-ylamino)-1,2-dihydro-2,7-naphthyridin-1-one; 2-methyl-8-{[(2R)-oxolan-2-ylmethyl]amino}-6-(pyrazin-2-ylamino)-1,2-dihydro-2,7-naphthyridin-1-one; 3-{[7-methyl-8-oxo-3-(pyrazin-2-ylamino)-7,8-dihydro-2,7-naphthyridin-1-yl]amino}benzamide; 4-{[7-methyl-8-oxo-3-(pyrazin-2-ylamino)-7,8-dihydro-2,7-naphthyridin-1-yl]amino}benzamide; 2-methyl-8-({4-[(morpholin-4-yl)carbonyl]phenyl}amino)-6-(pyrazin-2-ylamino)-1,2-dihydro-2,7-naphthyridin-1-one; 6-[(4-chloropyridin-2-yl)amino]-8-[(4-hydroxycyclohexyl)amino]-2-methyl-1,2-dihydro-2,7-naphthyridin-1-one; N-ethyl-4-({7-methyl-3-[(4-methylpyridin-2-yl)amino]-8-oxo-7,8-dihydro-2,7-naphthyridin-1-yl}amino)benzamide; N-ethyl-4-{[7-methyl-8-oxo-3-(pyrazin-2-ylamino)-7,8-dihydro-2,7-naphthyridin-1-yl]amino}benzamide; 4-({3-[(4-chloropyridin-2-yl)amino]-7-methyl-8-oxo-7,8-dihydro-2,7-naphthyridin-1-yl}amino)-N-ethylbenzamide; 6-[(4-chloropyridin-2-yl)amino]-2-ethyl-8-[(4-hydroxycyclohexyl)amino]-1,2-dihydro-2,7-naphthyridin-1-one; 6-[(4-chloropyridin-2-yl)amino]-8-[(4-hydroxycyclohexyl)amino]-2-(2-hydroxyethyl)-1,2-dihydro-2,7-naphthyridin-1-one; 6-[(4-chloropyridin-2-yl)amino]-8-[(4-hydroxycyclohexyl)amino]-2-methyl-1,2-dihydro-2,7-naphthyridin-1-one; 2-[2-(dimethylamino)ethyl]-8-[(4-hydroxycyclohexyl)amino]-6-[(4-methylpyridin-2-yl)amino]-1,2-dihydro-2,7-naphthyridin-1-one; 2-[2-(dimethylamino)ethyl]-8-[(4-hydroxycyclohexyl)amino]-6-(pyrazin-2-ylamino)-1,2-dihydro-2,7-naphthyridin-1-one; 6-[(4-chloropyridin-2-yl)amino]-2-[2-(dimethylamino)ethyl]-8-[(4-hydroxycyclohexyl)amino]-1,2-dihydro-2,7-naphthyridin-1-one; 8-[(3-methanesulfonylphenyl)amino]-2-methyl-6-[(4-methylpyridin-2-yl)amino]-1,2-dihydro-2,7-naphthyridin-1-one; 8-[(3-methanesulfonylphenyl)amino]-2-methyl-6-(pyrazin-2-ylamino)-1,2-dihydro-2,7-naphthyridin-1-one; 8-[(4-hydroxycyclohexyl)amino]-2-(2-hydroxyethyl)-6-[(4-methylpyridin-2-yl)amino]-1,2-dihydro-2,7-naphthyridin-1-one; 2-ethyl-8-[(4-hydroxycyclohexyl)amino]-6-[(5-methylpyrazin-2-yl)amino]-1,2-dihydro-2,7-naphthyridin-1-one; 2-ethyl-6-[(5-fluoro-4-methylpyridin-2-yl)amino]-8-[(4-hydroxycyclohexyl)amino]-1,2-dihydro-2,7-naphthyridin-1-one; 2-ethyl-8-{[(2S)-1-hydroxypropan-2-yl]amino}-6-[(5-methylpyrazin-2-yl)amino]-1,2-dihydro-2,7-naphthyridin-1-one; 2-ethyl-6-[(5-fluoro-4-methylpyridin-2-yl)amino]-8-{[(2S)-1-hydroxypropan-2-yl]amino}-1,2-dihydro-2,7-naphthyridin-1-one; 2-[2-(dimethylamino)ethyl]-8-[(4-methanesulfonylphenyl)amino]-6-[(4-methylpyridin-2-yl)amino]-1,2-dihydro-2,7-naphthyridin-1-one; 2-[2-(dimethylamino)ethyl]-8-[(4-methanesulfonylphenyl)amino]-6-(pyrazin-2-ylamino)-1,2-dihydro-2,7-naphthyridin-1-one; 8-{[(2S)-1-hydroxypropan-2-yl]amino}-2-methyl-6-[(4-methylpyridin-2-yl)amino]-1,2-dihydro-2,7-naphthyridin-1-one; 8-(cyclopropylamino)-2-methyl-6-(pyrazin-2-ylamino)-1,2-dihydro-2,7-naphthyridin-1-one; 6-[(4-chloropyridin-2-yl)amino]-8-(cyclopropylamino)-2-methyl-1,2-dihydro-2,7-naphthyridin-1-one; 6-[(5-chloro-4-methylpyridin-2-yl)amino]-8-(cyclopropylamino)-2-methyl-1,2-dihydro-2,7-naphthyridin-1-one; 8-(cyclopropylamino)-6-[(5-fluoro-4-methylpyridin-2-yl)amino]-2-methyl-1,2-dihydro-2,7-naphthyridin-1-one; 6-[(4-chloropyridin-2-yl)amino]-8-(cyclobutylamino)-2-methyl-1,2-dihydro-2,7-naphthyridin-1-one; 8-(tert-butylamino)-2-methyl-6-(pyrazin-2-ylamino)-1,2-dihydro-2,7-naphthyridin-1-one; 8-(tert-butylamino)-6-[(4-chloropyridin-2-yl)amino]-2-methyl-1,2-dihydro-2,7-naphthyridin-1-one; 8-(tert-butylamino)-6-[(5-chloro-4-methylpyridin-2-yl)amino]-2-methyl-1,2-dihydro-2,7-naphthyridin-1-one; 8-(tert-butylamino)-6-[(5-fluoro-4-methylpyridin-2-yl)amino]-2-methyl-1,2-dihydro-2,7-naphthyridin-1-one; 6-[(5-chloro-4-methylpyridin-2-yl)amino]-8-[(1-hydroxy-2-methylpropan-2-yl)amino]-2-(2-hydroxyethyl)-1,2-dihydro-2,7-naphthyridin-1-one; 6-[(5-fluoro-4-methylpyridin-2-yl)amino]-8-[(1-hydroxy-2-methylpropan-2-yl)amino]-2-(2-hydroxyethyl)-1,2-dihydro-2,7-naphthyridin-1-one; 8-[(1-hydroxy-2-methylpropan-2-yl)amino]-2-methyl-6-[(4-methylpyridin-2-yl)amino]-1,2-dihydro-2,7-naphthyridin-1-one; 6-[(5-fluoro-4-methylpyridin-2-yl)amino]-8-[(1-hydroxy-2-methylpropan-2-yl)amino]-2-methyl-1,2-dihydro-2,7-naphthyridin-1-one; 2-methyl-8-{[(2S)-oxolan-2-ylmethyl]amino}-6-(pyrazin-2-ylamino)-1,2-dihydro-2,7-naphthyridin-1-one; 2-ethyl-6-(pyrazin-2-ylamino)-8-[(2,2,2-trifluoroethyl)amino]-1,2-dihydro-2,7-naphthyridin-1-one; 8-(propan-2-ylamino)-6-{[4-(pyridin-3-yl)pyridin-2-yl]amino}-1,2-dihydro-2,7-naphthyridin-1-one; 2-methyl-8-(propan-2-ylamino)-6-{[4-(pyridin-3-yl)pyridin-2-yl]amino}-1,2-dihydro-2,7-naphthyridin-1-one; 2-ethyl-8-{[(2S)-1-hydroxybutan-2-yl]amino}-6-(pyrazin-2-ylamino)-1,2-dihydro-2,7-naphthyridin-1-one; 2-ethyl-8-{[(2S)-1-hydroxy-3-methylbutan-2-yl]amino}-6-[(4-methylpyridin-2-yl)amino]-1,2-dihydro-2,7-naphthyridin-1-one; 2-ethyl-8-{[(2S)-1-hydroxy-3-methylbutan-2-yl]amino}-6-(pyrazin-2-ylamino)-1,2-dihydro-2,7-naphthyridin-1-one; 8-{[(2S)-1-hydroxypropan-2-yl]amino}-2-methyl-6-(pyrazin-2-ylamino)-1,2-dihydro-2,7-naphthyridin-1-one; 6-[(4-chloropyridin-2-yl)amino]-8-{[(2S)-1-hydroxypropan-2-yl]amino}-2-methyl-1,2-dihydro-2,7-naphthyridin-1-one; 2-(2-hydroxyethyl)-8-[(4-methanesulfonylphenyl)amino]-6-[(4-methylpyridin-2-yl)amino]-1,2-dihydro-2,7-naphthyridin-1-one; 2-(2-hydroxyethyl)-8-[(4-methanesulfonylphenyl)amino]-6-(pyrazin-2-ylamino)-1,2-dihydro-2,7-naphthyridin-1-one; 2-ethyl-8-{[(1S,2S)-2-hydroxycyclohexyl]amino}-6-[(4-methylpyridin-2-yl)amino]-1,2-dihydro-2,7-naphthyridin-1-one; 2-ethyl-8-{[(1S,2S)-2-hydroxycyclohexyl]amino}-6-(pyrazin-2-ylamino)-1,2-dihydro-2,7-naphthyridin-1-one; 2-ethyl-8-{[(1S,2R)-2-hydroxycyclohexyl]amino}-6-[(4-methylpyridin-2-yl)amino]-1,2-dihydro-2,7-naphthyridin-1-one; 2-ethyl-8-{[(1S,2R)-2-hydroxycyclohexyl]amino}-6-(pyrazin-2-ylamino)-1,2-dihydro-2,7-naphthyridin-1-one; 6-[(4-chloropyridin-2-yl)amino]-2-methyl-8-{[(2S)-oxolan-2-ylmethyl]amino}-1,2-dihydro-2,7-naphthyridin-1-one; 2-ethyl-8-({[(1S,3S)-3-hydroxycyclopentyl]methyl}amino)-6-[(4-methylpyridin-2-yl)amino]-1,2-dihydro-2,7-naphthyridin-1-one; 2-ethyl-8-({[(1S,3S)-3-hydroxycyclopentyl]methyl}amino)-6-(pyrazin-2-ylamino)-1,2-dihydro-2,7-naphthyridin-1-one; 2-ethyl-8-({[(1R,3R)-3-hydroxycyclopentyl]methyl}amino)-6-[(4-methylpyridin-2-yl)amino]-1,2-dihydro-2,7-naphthyridin-1-one; 2-ethyl-8-({[(1R,3R)-3-hydroxycyclopentyl]methyl}amino)-6-(pyrazin-2-ylamino)-1,2-dihydro-2,7-naphthyridin-1-one; 6-[(4-chloropyridin-2-yl)amino]-2-ethyl-8-[(2,2,2-trifluoroethyl)amino]-1,2-dihydro-2,7-naphthyridin-1-one; 2-(2-hydroxyethyl)-6-(pyrazin-2-ylamino)-8-[(2,2,2-trifluoroethyl)amino]-1,2-dihydro-2,7-naphthyridin-1-one; 6-[(4-chloropyridin-2-yl)amino]-2-(2-hydroxyethyl)-8-[(2,2,2-trifluoroethyl)amino]-1,2-dihydro-2,7-naphthyridin-1-one; 6-[(4-phenylpyridin-2-yl)amino]-8-(propan-2-ylamino)-1,2-dihydro-2,7-naphthyridin-1-one; 6-{[4-(1-methyl-1H-pyrazol-4-yl)pyridin-2-yl]amino}-8-(propan-2-ylamino)-1,2-dihydro-2,7-naphthyridin-1-one; 2-methyl-6-{[4-(1-methyl-1H-pyrazol-4-yl)pyridin-2-yl]amino}-8-(propan-2-ylamino)-1,2-dihydro-2,7-naphthyridin-1-one; 2-ethyl-8-{[(2S)-1-hydroxybutan-2-yl]amino}-6-[(4-methylpyridin-2-yl)amino]-1,2-dihydro-2,7-naphthyridin-1-one; 2-ethyl-8-{[(2R)-1-hydroxybutan-2-yl]amino}-6-[(4-methylpyridin-2-yl)amino]-1,2-dihydro-2,7-naphthyridin-1-one; 2-ethyl-8-{[(2R)-1-hydroxybutan-2-yl]amino}-6-(pyrazin-2-ylamino)-1,2-dihydro-2,7-naphthyridin-1-one; 2-(2-hydroxyethyl)-8-[(1-methylcyclobutyl)amino]-6-[(4-methylpyridin-2-yl)amino]-1,2-dihydro-2,7-naphthyridin-1-one; 2-(2-hydroxyethyl)-8-[(1-methylcyclobutyl)amino]-6-(pyrazin-2-ylamino)-1,2-dihydro-2,7-naphthyridin-1-one; 2-ethyl-8-({[(1R,3S)-3-hydroxy-3-methylcyclopentyl]methyl}amino)-6-[(4-methylpyridin-2-yl)amino]-1,2-dihydro-2,7-naphthyridin-1-one; 2-ethyl-8-({[(1R,3S)-3-hydroxy-3-methylcyclopentyl]methyl}amino)-6-(pyrazin-2-ylamino)-1,2-dihydro-2,7-naphthyridin-1-one; 2-ethyl-8-({[(1S,3R)-3-hydroxy-3-methylcyclopentyl]methyl}amino)-6-[(4-methylpyridin-2-yl)amino]-1,2-dihydro-2,7-naphthyridin-1-one; 2-ethyl-8-({[(1S,3R)-3-hydroxy-3-methylcyclopentyl]methyl}amino)-6-(pyrazin-2-ylamino)-1,2-dihydro-2,7-naphthyridin-1-one; 2-ethyl-8-{[(1R,2S)-2-hydroxycyclopentyl]amino}-6-[(4-methylpyridin-2-yl)amino]-1,2-dihydro-2,7-naphthyridin-1-one; 2-ethyl-8-{[(1R,2S)-2-hydroxycyclopentyl]amino}-6-(pyrazin-2-ylamino)-1,2-dihydro-2,7-naphthyridin-1-one; 2-ethyl-8-{[(1S,2R)-2-hydroxycyclopentyl]amino}-6-[(4-methylpyridin-2-yl)amino]-1,2-dihydro-2,7-naphthyridin-1-one; 2-ethyl-8-{[(1S,2R)-2-hydroxycyclopentyl]amino}-6-(pyrazin-2-ylamino)-1,2-dihydro-2,7-naphthyridin-1-one; 2-ethyl-8-({[(1S,2S)-2-hydroxycyclopentyl]amino}-6-[(4-methylpyridin-2-yl)amino]-1,2-dihydro-2,7-naphthyridin-1-one; 2-ethyl-8-{[(1S,2S)-2-hydroxycyclopentyl]amino}-6-(pyrazin-2-ylamino)-1,2-dihydro-2,7-naphthyridin-1-one; 2-ethyl-8-{[(1S,3R)-3-hydroxycyclopentyl]amino}-6-[(4-methylpyridin-2-yl)amino]-1,2-dihydro-2,7-naphthyridin-1-one; 2-ethyl-8-{[(1S,3R)-3-hydroxycyclopentyl]amino}-6-(pyrazin-2-ylamino)-1,2-dihydro-2,7-naphthyridin-1-one; 2-ethyl-8-{[(1R,3S)-3-hydroxycyclopentyl]amino}-6-[(4-methylpyridin-2-yl)amino]-1,2-dihydro-2,7-naphthyridin-1-one; 2-ethyl-8-{[(1R,3S)-3-hydroxycyclopentyl]amino}-6-(pyrazin-2-ylamino)-1,2-dihydro-2,7-naphthyridin-1-one; 8-(tert-butylamino)-6-({4-[(3-hydroxyazetidin-1-yl)methyl]pyridin-2-yl}amino)-2-(2-hydroxyethyl)-1,2-dihydro-2,7-naphthyridin-1-one; 8-(tert-butylamino)-6-({4-[(3-hydroxy-3-methylazetidin-1-yl)methyl]pyridin-2-yl}amino)-2-(2-hydroxyethyl)-1,2-dihydro-2,7-naphthyridin-1-one; 8-(tert-butylamino)-2-(2-hydroxyethyl)-6-{[4-(2,2,2-trifluoro-1-hydroxyethyl)pyridin-2-yl]amino}-1,2-dihydro-2,7-naphthyridin-1-one; 8-(tert-butylamino)-2-(2-hydroxyethyl)-6-{[4-(morpholin-4-ylmethyl)pyridin-2-yl]amino}-1,2-dihydro-2,7-naphthyridin-1-one; 8-(tert-butylamino)-2-(2-hydroxyethyl)-6-{[4-(piperidin-1-ylmethyl)pyridin-2-yl]amino}-1,2-dihydro-2,7-naphthyridin-1-one; 8-(tert-butylamino)-2-(2-hydroxyethyl)-6-({4-[(oxetan-3-ylamino)methyl]pyridin-2-yl}amino)-1,2-dihydro-2,7-naphthyridin-1-one; 8-(tert-butylamino)-2-(2-hydroxyethyl)-6-[(5-methoxypyridin-2-yl)amino]-1,2-dihydro-2,7-naphthyridin-1-one; 8-(tert-butylamino)-2-(2-hydroxyethyl)-6-{[6-(hydroxymethyl)pyrimidin-4-yl]amino}-1,2-dihydro-2,7-naphthyridin-1-one; 8-(tert-butylamino)-2-(2-hydroxyethyl)-6-[(4-{[(2-hydroxyethyl)amino]methyl}pyridin-2-yl)amino]-1,2-dihydro-2,7-naphthyridin-1-one; 8-(tert-butylamino)-2-(2-hydroxyethyl)-6-{[4-(3-hydroxyoxetan-3-yl)pyridin-2-yl]amino}-1,2-dihydro-2,7-naphthyridin-1-one; 8-(tert-butylamino)-6-({5-fluoro-4-[(3-hydroxy-3-methylazetidin-1-yl)methyl]pyridin-2-yl}amino)-2-(2-hydroxyethyl)-1,2-dihydro-2,7-naphthyridin-1-one; 8-(tert-butylamino)-6-({5-fluoro-4-[(3-hydroxyazetidin-1-yl)methyl]pyridin-2-yl}amino)-2-(2-hydroxyethyl)-1,2-dihydro-2,7-naphthyridin-1-one; 6-{[4-(2-amino-1-hydroxyethyl)pyridin-2-yl]amino}-8-(tert-butylamino)-2-(2-hydroxyethyl)-1,2-dihydro-2,7-naphthyridin-1-one; 2-[8-(tert-butylamino)-1-oxo-6-{[4-(2,2,2-trifluoro-1-hydroxyethyl)pyridin-2-yl]amino}-1,2-dihydro-2,7-naphthyridin-2-yl]acetamide; 2-{8-[(1-methylcyclopropyl)amino]-1-oxo-6-{[4-(2,2,2-trifluoro-1-hydroxyethyl)pyridin-2-yl]amino}-1,2-dihydro-2,7-naphthyridin-2-yl}acetamide; 8-(tert-butylamino)-6-{[4-(difluoromethyl)pyridin-2-yl]amino}-2-(2-hydroxyethyl)-1,2-dihydro-2,7-naphthyridin-1-one; 8-(tert-butylamino)-6-[(4-tert-butylpyridin-2-yl)amino]-2-(2-hydroxyethyl)-1,2-dihydro-2,7-naphthyridin-1-one; 2-(6-{[4-(3-hydroxyoxetan-3-yl)pyridin-2-yl]amino}-8-[(1-methylcyclopropyl)amino]-1-oxo-1,2-dihydro-2,7-naphthyridin-2-yl)acetamide; 8-(tert-butylamino)-6[(6-ethenylpyrimidin-4-yl)amino]-2-(2-hydroxyethyl)-1,2-dihydro-2,7-naphthyridin-1-one; 8-(tert-butylamino)-6-[(6-ethylpyrimidin-4-yl)amino]-2-(2-hydroxyethyl)-1,2-dihydro-2,7-naphthyridin-1-one; 2-(6-{[6-(2-hydroxypropan-2-yl)pyrimidin-4-yl]amino}-8-[(1-methylcyclopropyl)amino]-1-oxo-1,2-dihydro-2,7-naphthyridin-2-yl)acetamide; 8-(tert-butylamino)-2-(2-hydroxyethyl)-6-({6-[(1E)-3-methoxyprop-1-en-1-yl]pyrimidin-4-yl}amino)-1,2-dihydro-2,7-naphthyridin-1-one; 8-(tert-butylamino)-2-(2-hydroxyethyl)-6-{[6-(3-methoxypropyl)pyrimidin-4-yl]amino}-1,2-dihydro-2,7-naphthyridin-1-one; 8-(tert-butylamino)-6-{[6-(cyclohex-1-en-1-yl)pyrimidin-4-yl]amino}-2-(2-hydroxyethyl)-1,2-dihydro-2,7-naphthyridin-1-one; 8-(tert-butylamino)-6-{[6-(3,4-dihydro-2H-pyran-6-yl)pyrimidin-4-yl]amino}-2-(2-hydroxyethyl)-1,2-dihydro-2,7-naphthyridin-1-one; 8-(tert-butylamino)-6-[(6-cyclohexylpyrimidin-4-yl)amino]-2-(2-hydroxyethyl)-1,2-dihydro-2,7-naphthyridin-1-one; 8-(tert-butylamino)-2-(2-hydroxyethyl)-6-{[6-(oxan-2-yl)pyrimidin-4-yl]amino}-1,2-dihydro-2,7-naphthyridin-1-one; 8-(tert-butylamino)-6-({6-[(1E)-4-hydroxybut-1-en-1-yl]pyrimidin-4-yl}amino)-2-(2-hydroxyethyl)-1,2-dihydro-2,7-naphthyridin-1-one; 8-(tert-butylamino)-2-(2-hydroxyethyl)-6-{[4-(1,1,1-trifluoro-2-hydroxypropan-2-yl)pyridin-2-yl]amino}-1,2-dihydro-2,7-naphthyridin-1-one; 8-(tert-butylamino)-6-{[6-(4-hydroxybutyl)pyrimidin-4-yl]amino}-2-(2-hydroxyethyl)-1,2-dihydro-2,7-naphthyridin-1-one; 8-(tert-butylamino)-6-{[6-(3-hydroxy-3-methylazetidin-1-yl)pyrimidin-4-yl]amino}-2-(2-hydroxyethyl)-1,2-dihydro-2,7-naphthyridin-1-one; 8-(tert-butylamino)-6-{[6-(3-hydroxyazetidin-1-yl)pyrimidin-4-yl]amino}-2-(2-hydroxyethyl)-1,2-dihydro-2,7-naphthyridin-1-one; 8-(tert-butylamino)-2-(2-hydroxyethyl)-6-{[6-(4-oxopiperidin-1-yl)pyrimidin-4-yl]amino}-1,2-dihydro-2,7-naphthyridin-1-one; 6-[(6-acetylpyrimidin-4-yl)amino]-8-(tert-butylamino)-2-(2-hydroxyethyl)-1,2-dihydro-2,7-naphthyridin-1-one; 8-(tert-butylamino)-2-(2-hydroxyethyl)-6-{[6-(4-hydroxypiperidin-1-yl)pyrimidin-4-yl]amino}-1,2-dihydro-2,7-naphthyridin-1-one; 8-(tert-butylamino)-2-(2-hydroxyethyl)-6-({6-[3-(trifluoromethyl)-1H-pyrazol-4-yl]pyrimidin-4-yl}amino)-1,2-dihydro-2,7-naphthyridin-1-one; 8-(tert-butylamino)-2-(2-hydroxyethyl)-6-{[6-(1-methyl-1H-pyrazol-4-yl)pyrimidin-4-yl]amino}-1,2-dihydro-2,7-naphthyridin-1-one; 8-(tert-butylamino)-2-(2-hydroxyethyl)-6-{[6-(2-methoxypyrimidin-5-yl)pyrimidin-4-yl]amino}-1,2-dihydro-2,7-naphthyridin-1-one; 8-(tert-butylamino)-2-(2-hydroxyethyl)-6-{[6-(piperidin-4-yl)pyrimidin-4-yl]amino}-1,2-dihydro-2,7-naphthyridin-1-one; 8-(tert-butylamino)-2-(2-hydroxyethyl)-6-{[6-(1H-pyrazol-4-yl)pyrimidin-4-yl]amino}-1,2-dihydro-2,7-naphthyridin-1-one; 8-(tert-butylamino)-2-(2-hydroxyethyl)-6-({6-[1-(2-hydroxyethyl)piperidin-4-yl]pyrimidin-4-yl}amino)-1,2-dihydro-2,7-naphthyridin-1-one; 8-(tert-butylamino)-6-{[6-(2-hydroxy-2-methylpropoxy)pyrimidin-4-yl]amino}-2-(2-hydroxyethyl)-1,2-dihydro-2,7-naphthyridin-1-one; 8-(tert-butylamino)-2-[(2S)-2,3-dihydroxypropyl]-6-{[6-(2-hydroxyethoxy)pyrimidin-4-yl]amino}-1,2-dihydro-2,7-naphthyridin-1-one; 8-(tert-butylamino)-2-[(2S)-2,3-dihydroxypropyl]-6-[(6-[(2S)-1-hydroxypropan-2-yl]oxy pyrimidin-4-yl)amino]-1,2-dihydro-2,7-naphthyridin-1-one; 8-(tert-butylamino)-2-[(2S)-2,3-dihydroxypropyl]-6-{[6-(piperidin-4-yloxy)pyrimidin-4-yl]amino}-1,2-dihydro-2,7-naphthyridin-1-one; 8-(tert-butylamino)-2-(2-hydroxyethyl)-6-{[6-(piperidin-4-yloxy)pyrimidin-4-yl]amino}-1,2-dihydro-2,7-naphthyridin-1-one; 8-(tert-butylamino)-2-(2-hydroxyethyl)-6-[(6-{[1-(2-hydroxyethyl)piperidin-4-yl]oxy}pyrimidin-4-yl)amino]-1,2-dihydro-2,7-naphthyridin-1-one; 8-(tert-butylamino)-2-(2-hydroxyethyl)-6-[(6-{[1-(2-methoxyethyl)piperidin-4-yl]oxy}pyrimidin-4-yl)amino]-1,2-dihydro-2,7-naphthyridin-1-one; 8-(tert-butylamino)-2-(2-hydroxyethyl)-6-({6-[(1-methylpiperidin-4-yl)oxy]pyrimidin-4-yl}amino)-1,2-dihydro-2,7-naphthyridin-1-one; 8-(tert-butylamino)-2-[(2S)-2,3-dihydroxypropyl]-6-({6-[(1-methylpiperidin-4-yl)oxy]pyrimidin-4-yl}amino)-1,2-dihydro-2,7-naphthyridin-1-one; 2-(2-hydroxyethyl)-8-({3-methyl-4-[(morpholin-4-yl)carbonyl]phenyl}amino)-6-[(4-methylpyridin-2-yl)amino]-1,2-dihydro-2,7-naphthyridin-1-one; N-(3-methoxypropyl)-N-methyl-4-({7-methyl-3-[(4-methylpyridin-2-yl)amino]-8-oxo-7,8-dihydro-2,7-naphthyridin-1-yl}amino)benzene-1-sulfonamide; N-(2-methoxyethyl)-4-({7-methyl-3-[(4-methylpyridin-2-yl)amino]-8-oxo-7,8-dihydro-2,7-naphthyridin-1-yl}amino)benzamide; 2-methyl-6-[(4-methylpyridin-2-yl)amino]-8-({4-[2-(morpholin-4-yl)ethoxy]phenyl}amino)-1,2-dihydro-2,7-naphthyridin-1-one; 2-methyl-5-({7-methyl-3-[(4-methylpyridin-2-yl)amino]-8-oxo-7,8-dihydro-2,7-naphthyridin-1-yl}amino)benzene-1-sulfonamide; 2-(2-hydroxyethyl)-8-[(2-methyl-2H-indazol-6-yl)amino]-6-(pyrazin-2-ylamino)-1,2-dihydro-2,7-naphthyridin-1-one; 8-[(2,3-dimethyl-2H-indazol-6-yl)amino]-2-(2-hydroxyethyl)-6-(pyrazin-2-ylamino)-1,2-dihydro-2,7-naphthyridin-1-one; 6-[(4-hydroxycyclohexyl)amino]-8-{[4-(morpholin-4-yl)phenyl]amino}-1,2-dihydro-2,7-naphthyridin-1-one; 3-{[7-(2-hydroxyethyl)-1-[(2-methyl-2H-indazol-5-yl)amino]-8-oxo-7,8-dihydro-2,7-naphthyridin-3-yl]amino}pyrazin-1-ium-1-olate; 2-(2-hydroxyethyl)-8-[(2-methyl-2H-indazol-5-yl)amino]-6-(pyrazin-2-ylamino)-1,2-dihydro-2,7-naphthyridin-1-one; 2-[8-(tert-butylamino)-1-oxo-6-(pyrazin-2-ylamino)-1,2-dihydro-2,7-naphthyridin-2-yl]propanamide; 3-[8-(tert-butylamino)-1-oxo-6-(pyrazin-2-ylamino)-1,2-dihydro-2,7-naphthyridin-2-yl]propanamide; 2-[8-(tert-butylamino)-1-oxo-6-(pyrazin-2-ylamino)-1,2-dihydro-2,7-naphthyridin-2-yl]acetamide; 2-[6-(2-aminopyrimidin-5-yl)-8-(tert-butylamino)-1-oxo-1,2-dihydro-2,7-naphthyridin-2-yl]acetamide; 8-(tert-butylamino)-2-(2,3-dihydroxypropyl)-6-(pyrazin-2-ylamino)-1,2-dihydro-2,7-naphthyridin-1-one; 2-[6-(2-aminopyrimidin-5-yl)-8-(tert-butylamino)-1-oxo-1,2-dihydro-2,7-naphthyridin-2-yl]propanamide; 6-(2-aminopyrimidin-5-yl)-8-(tert-butylamino)-4-chloro-2-(2-hydroxyethyl)-1,2-dihydro-2,7-naphthyridin-1-one; 2-[6-(2-aminopyrimidin-5-yl)-8-(tert-butylamino)-1-oxo-1,2-dihydro-2,7-naphthyridin-2-yl]acetic acid; 8-(tert-butylamino)-2-[(2S)-2,3-dihydroxypropyl]-6-(pyrazin-2-ylamino)-1,2-dihydro-2,7-naphthyridin-1-one; 8-(tert-butylamino)-2-[(2R)-2,3-dihydroxypropyl]-6-(pyrazin-2-ylamino)-1,2-dihydro-2,7-naphthyridin-1-one; 8-[(2-cyclopropylpropan-2-yl)amino]-2-(2-hydroxyethyl)-6-(pyrazin-2-ylamino)-1,2-dihydro-2,7-naphthyridin-1-one; 6-(2-aminopyrimidin-5-yl)-8-(tert-butylamino)-2-(2-hydroxyethyl)-4-methyl-1,2-dihydro-2,7-naphthyridin-1-one; 2-[8-(tert-butylamino)-1-oxo-6-(pyrazin-2-ylamino)-1,2-dihydro-2,7-naphthyridin-2-yl]-2-methylpropanamide; 2-[6-(2-aminopyrimidin-5-yl)-8-(tert-butylamino)-1-oxo-1,2-dihydro-2,7-naphthyridin-2-yl]-2-methylpropanamide; 3-{[1-(tert-butylamino)-7-[(2S)-2,3-dihydroxypropyl]-8-oxo-7,8-dihydro-2,7-naphthyridin-3-yl]amino}pyrazin-1-ium-1-olate; 3-{[1-(tert-butylamino)-7-[(2R)-2,3-dihydroxypropyl]-8-oxo-7,8-dihydro-2,7-naphthyridin-3-yl]amino}pyrazin-1-ium-1-olate; 8-(tert-butylamino)-2-[(2S)-2-hydroxy-2-phenylethyl]-6-(pyrazin-2-ylamino)-1,2-dihydro-2,7-naphthyridin-1-one; 8-(tert-butylamino)-2-(2-hydroxy-3-methoxypropyl)-6-(pyrazin-2-ylamino)-1,2-dihydro-2,7-naphthyridin-1-one; 6-(2-aminopyrimidin-5-yl)-8-(tert-butylamino)-2-[2-(morpholin-4-yl)-2-oxoethyl]-1,2-dihydro-2,7-naphthyridin-1-one; 8-(tert-butylamino)-6-(pyrazin-2-ylamino)-1,2-dihydro-2,7-naphthyridin-1-one; 2-{[1-(tert-butylamino)-7-(2-hydroxyethyl)-8-oxo-7,8-dihydro-2,7-naphthyridin-3-yl]amino}pyridine-4-carboxylic acid; 2-{[1-(tert-butylamino)-7-(2-hydroxyethyl)-8-oxo-7,8-dihydro-2,7-naphthyridin-3-yl]amino}pyridine-4-carboxamide; 2-{[1-(tert-butylamino)-7-(2-hydroxyethyl)-8-oxo-7,8-dihydro-2,7-naphthyridin-3-yl]amino}-N-(2-methoxyethyl)pyridine-4-carboxamide; 2-{[1-(tert-butylamino)-7-(2-hydroxyethyl)-8-oxo-7,8-dihydro-2,7-naphthyridin-3-yl]amino}-N-[2-(dimethylamino)ethyl]pyridine-4-carboxamide; 6-[(4-acetylpyridin-2-yl)amino]-8-(tert-butylamino)-2-(2-hydroxyethyl)-1,2-dihydro-2,7-naphthyridin-1-one; 8-(tert-butylamino)-2-(2-hydroxyethyl)-6-({4-[(1E)-1-(hydroxyimino)ethyl]pyridin-2-yl}amino)-1,2-dihydro-2,7-naphthyridin-1-one; 8-(tert-butylamino)-6-({4-[(1E)-1-{[2-(dimethylamino)ethoxy]imino}ethyl]pyridin-2-yl}amino)-2-(2-hydroxyethyl)-1,2-dihydro-2,7-naphthyridin-1-one; 2-{[1-(tert-butylamino)-7-(2-hydroxyethyl)-8-oxo-7,8-dihydro-2,7-naphthyridin-3-yl]amino}-N-[2-(1H-imidazol-4-yl)ethyl]pyridine-4-carboxamide; 8-(tert-butylamino)-2-(2-hydroxyethyl)-6-[(4-{[3-(hydroxymethyl)piperidin-1-yl]carbonyl}pyridin-2-yl)amino]-1,2-dihydro-2,7-naphthyridin-1-one; 8-(tert-butylamino)-2-(2-hydroxyethyl)-6-{[4-(1,2-oxazol-5-yl)pyridin-2-yl]amino}-1,2-dihydro-2,7-naphthyridin-1-one; 8-(tert-butylamino)-2-(2-hydroxyethyl)-6-{[4-(1H-pyrazol-3-yl)pyridin-2-yl]amino}-1,2-dihydro-2,7-naphthyridin-1-one; 6-{[4-(2-aminopyrimidin-4-yl)pyridin-2-yl]amino}-8-(tert-butylamino)-2-(2-hydroxyethyl)-1,2-dihydro-2,7-naphthyridin-1-one; 8-(tert-butylamino)-2-(2-hydroxyethyl)-6-(1,3-thiazol-2-ylamino)-1,2-dihydro-2,7-naphthyridin-1-one; 8-(tert-butylamino)-2-(2-hydroxyethyl)-6-{[4-(hydroxymethyl)pyridin-2-yl]amino}-1,2-dihydro-2,7-naphthyridin-1-one; 8-(tert-butylamino)-2-(2-hydroxyethyl)-6-[(4-{[(2-methoxyethyl)amino]methyl}pyridin-2-yl)amino]-1,2-dihydro-2,7-naphthyridin-1-one; 8-(tert-butylamino)-2-(2-hydroxyethyl)-6-({4-[(1E)-1-(methoxyimino)ethyl]pyridin-2-yl}amino)-1,2-dihydro-2,7-naphthyridin-1-one; 8-(tert-butylamino)-6-[(5-fluoropyridin-2-yl)amino]-2-(2-hydroxyethyl)-1,2-dihydro-2,7-naphthyridin-1-one; 6-{[1-(tert-butylamino)-7-(2-hydroxyethyl)-8-oxo-7,8-dihydro-2,7-naphthyridin-3-yl]amino}pyridine-3-carbonitrile; 8-(tert-butylamino)-2-(2-hydroxyethyl)-6-{[4-(2-hydroxypropan-2-yl)pyridin-2-yl]amino}-1,2-dihydro-2,7-naphthyridin-1-one; 8-(tert-butylamino)-2-(2-hydroxyethyl)-6-[(4-methanesulfonylpyridin-2-yl)amino]-1,2-dihydro-2,7-naphthyridin-1-one; 8-(tert-butylamino)-2-(2-hydroxyethyl)-6-{[4-(1-hydroxyethyl)pyridin-2-yl]amino}-1,2-dihydro-2,7-naphthyridin-1-one; 2-ethyl-8-[(1-hydroxy-2-methylpropan-2-yl)amino]-6-{[4-(2-hydroxypropan-2-yl)pyridin-2-yl]amino}-1,2-dihydro-2,7-naphthyridin-1-one; 2-ethyl-6-{[4-(2-hydroxypropan-2-yl)pyridin-2-yl]amino}-8-[(1-methylcyclobutyl)amino]-1,2-dihydro-2,7-naphthyridin-1-one; 2-ethyl-8-{[1-(hydroxymethyl)cyclobutyl]amino}-6-{[4-(2-hydroxypropan-2-yl)pyridin-2-yl]amino}-1,2-dihydro-2,7-naphthyridin-1-one; 2-(2-hydroxyethyl)-6-{[4-(2-hydroxypropan-2-yl)pyridin-2-yl]amino}-8-[(1-methylcyclobutyl)amino]-1,2-dihydro-2,7-naphthyridin-1-one; 2-(2-chydroxyethyl)-6-{[4-(2-hydroxypropan-2-yl)pyridin-2-yl]amino}-8-(propan-2-ylamino)-1,2-dihydro-2,7-naphthyridin-1-one; 8-[(4-hydroxycyclohexyl)amino]-6-{[4-(2-hydroxypropan-2-yl)pyridin-2-yl]amino}-2-(propan-2-yl)-1,2-dihydro-2,7-naphthyridin-1-one; 6-{[4-(aminomethyl)pyridin-2-yl]amino}-8-(tert-butylamino)-2-(2-hydroxyethyl)-1,2-dihydro-2,7-naphthyridin-1-one; 8-(tert-butylamino)-2-(2-hydroxyethyl)-6-{[4-(3-hydroxypentan-3-yl)pyridin-2-yl]amino}-1,2-dihydro-2,7-naphthyridin-1-one; 8-(tert-butylamino)-2-(2-hydroxyethyl)-6-{[4-(methoxymethyl)pyridin-2-yl]amino}-1,2-dihydro-2,7-naphthyridin-1-one; 8-(tert-butylamino)-2-(2-hydroxyethyl)-6-{[4-(1-hydroxypropyl)pyridin-2-yl]amino}-1,2-dihydro-2,7-naphthyridin-1-one; 6-{[4-(2-aminopropan-2-yl)pyridin-2-yl]amino}-8-(tert-butylamino)-2-(2-hydroxyethyl)-1,2-dihydro-2,7-naphthyridin-1-one; 6-{[4-(1-aminoethyl)pyridin-2-yl]amino}-8-(tert-butylamino)-2-(2-hydroxyethyl)-1,2-dihydro-2,7-naphthyridin-1-one; 8-(tert-butylamino)-2-(2-hydroxyethyl)-6-({4-[(1R)-1-hydroxyethyl]pyridin-2-yl}amino)-1,2-dihydro-2,7-naphthyridin-1-one; 8-(tert-butylamino)-2-(2-hydroxyethyl)-6-({4-[(1S)-1-hydroxyethyl]pyridin-2-yl}amino)-1,2-dihydro-2,7-naphthyridin-1-one; 8-(tert-butylamino)-2-(2-hydroxyethyl)-6-{[4-(1-hydroxyethyl)pyrimidin-2-yl]amino}-1,2-dihydro-2,7-naphthyridin-1-one; 8-(tert-butylamino)-2-(2-hydroxyethyl)-6-{[6-(2-hydroxypropan-2-yl)pyrimidin-4-yl]amino}-1,2-dihydro-2,7-naphthyridin-1-one; 8-(tert-butylamino)-6-{[5-fluoro-4-(2-hydroxypropan-2-yl)pyridin-2-yl]amino}-2-(2-hydroxyethyl)-1,2-dihydro-2,7-naphthyridin-1-one; 2-(2-hydroxyethyl)-6-{[4-(1-hydroxyethyl)pyridin-2-yl]amino}-8-[(1-methylcyclobutyl)amino]-1,2-dihydro-2,7-naphthyridin-1-one; 8-(tert-butylamino)-2-(2-hydroxyethyl)-6-{[4-(1-hydroxyethyl)pyridin-2-yl]amino}-1,2-dihydro-2,7-naphthyridin-1-one; 6-[6-amino-5-(hydroxymethyl)pyridin-3-yl]-8-(tert-butylamino)-2-(2-hydroxyethyl)-1,2-dihydro-2,7-naphthyridin-1-one; 2-(6-{[4-(1-hydroxyethyl)pyridin-2-yl]amino}-8-[(1-methylcyclobutyl)amino]-1-oxo-1,2-dihydro-2,7-naphthyridin-2-yl)acetamide; 2-(6-{[4-(2-hydroxypropan-2-yl)pyridin-2-yl]amino}-8-[(1-methylcyclobutyl)amino]-1-oxo-1,2-dihydro-2,7-naphthyridin-2-yl)acetamide; 2-(6-{[4-(1-hydroxyethyl)pyridin-2-yl]amino}-8-[(1-methylcyclopropyl)amino]-1-oxo-1,2-dihydro-2,7-naphthyridin-2-yl)acetamide; 2-(6-{[4-(2-hydroxypropan-2-yl)pyridin-2-yl]amino}-8-[(1-methylcyclopropyl)amino]-1-oxo-1,2-dihydro-2,7-naphthyridin-2-yl)acetamide; 2-[8-(tert-butylamino)-6-{[4-(1-hydroxyethyl)pyridin-2-yl]amino}-1-oxo-1,2-dihydro-2,7-naphthyridin-2-yl]acetamide; 2-[8-(tert-butylamino)-6-{[4-(2-hydroxypropan-2-yl)pyridin-2-yl]amino}-1-oxo-1,2-dihydro-2,7-naphthyridin-2-yl]acetamide; 8-(tert-butylamino)-2-(2-hydroxyethyl)-6-{[6-(1-hydroxyethyl)pyrimidin-4-yl]amino}-1,2-dihydro-2,7-naphthyridin-1-one; 8-(tert-butylamino)-6-{[5-chloro-4-(2-hydroxypropan-2-yl)pyridin-2-yl]amino}-2-(2-hydroxyethyl)-1,2-dihydro-2,7-naphthyridin-1-one; 2-(2,3-dihydroxypropyl)-6-{[6-(2-hydroxypropan-2-yl)pyrimidin-4-yl]amino}-8-[(1-methylcyclopropyl)amino]-1,2-dihydro-2,7-naphthyridin-1-one; 8-(tert-butylamino)-2-(2,3-dihydroxypropyl)-6-{[6-(2-hydroxypropan-2-yl)pyrimidin-4-yl]amino}-1,2-dihydro-2,7-naphthyridin-1-one; 8-(tert-butylamino)-2-(2-hydroxy-2-methylpropyl)-6-{[6-(2-hydroxypropan-2-yl)pyrimidin-4-yl]amino}-1,2-dihydro-2,7-naphthyridin-1-one; 2-(2,3-dihydroxypropyl)-6-{[6-(2-hydroxypropan-2-yl)pyrimidin-4-yl]amino}-8-[(1-methylcyclobutyl)amino]-1,2-dihydro-2,7-naphthyridin-1-one; 2-(2-hydroxy-2-methylpropyl)-6-{[6-(2-hydroxypropan-2-yl)pyrimidin-4-yl]amino}-8-[(1-methylcyclobutyl)amino]-1,2-dihydro-2,7-naphthyridin-1-one; 8-(tert-butylamino)-6-{[6-(2-hydroxypropan-2-yl)pyrimidin-4-yl]amino}-2-(oxetan-3-yl)-1,2-dihydro-2,7-naphthyridin-1-one; 8-(tert-butylamino)-2-(2-hydroxyethyl)-6-[(6-methoxypyrimidin-4-yl)amino]-1,2-dihydro-2,7-naphthyridin-1-one; 8-(tert-butylamino)-2-(2-hydroxyethyl)-6-(pyrimidin-4-ylamino)-1,2-dihydro-2,7-naphthyridin-1-one; 6-(2-aminopyrimidin-5-yl)-8-(tert-butylamino)-2-(2-methanesulfonylethyl)-1,2-dihydro-2,7-naphthyridin-1-one; 8-(tert-butylamino)-2-(2-methanesulfonylethyl)-6-(pyrazin-2-ylamino)-1,2-dihydro-2,7-naphthyridin-1-one; 8-(tert-butylamino)-6-{[6-(2-hydroxypropan-2-yl)pyrimidin-4-yl]amino}-2-(2-methanesulfonylethyl)-1,2-dihydro-2,7-naphthyridin-1-one; 8-(tert-butylamino)-2-[(2S)-2,3-dihydroxypropyl]-6-{[6-(2-hydroxypropan-2-yl)pyrimidin-4-yl]amino}-1,2-dihydro-2,7-naphthyridin-1-one; 8-(tert-butylamino)-2-(1,3-dihydroxypropan-2-yl)-6-{[6-(2-hydroxypropan-2-yl)pyrimidin-4-yl]amino}-1,2-dihydro-2,7-naphthyridin-1-one; 6-(2-aminopyrimidin-5-yl)-8-(oxan-4-ylamino)-1,2-dihydro-2,7-naphthyridin-1-one; 8-amino-6-(2-aminopyrimidin-5-yl)-2-ethyl-1,2-dihydro-2,7-naphthyridin-1-one; N-[3-(2-aminopyrimidin-5-yl)-7-ethyl-8-oxo-7,8-dihydro-2,7-naphthyridin-1-yl]methanesulfonamide; N-[7-ethyl-8-oxo-3-(pyrazin-2-ylamino)-7,8-dihydro-2,7-naphthyridin-1-yl]-methanesulfonamide; 2-[6-(2-aminopyrimidin-5-yl)-8-{[3-(hydroxymethyl)cyclobutyl]amino}-1-oxo-1,2-dihydro-2,7-naphthyridin-2-yl]acetamide; 2-(8-{[3-(hydroxymethyl)cyclobutyl]amino}-1-oxo-6-(pyrazin-2-ylamino)-1,2-dihydro-2,7-naphthyridin-2-yl)acetamide; 6-(2-aminopyrimidin-5-yl)-2-ethyl-8-{[3-(hydroxymethyl)cyclobutyl]amino}-1,2-dihydro-2,7-naphthyridin-1-one; 6-(2-aminopyrimidin-5-yl)-2-ethyl-8-{[(1R)-3-fluorocyclopentyl]amino}-1,2-dihydro-2,7-naphthyridin-1-one; 2-[6-(2-aminopyrimidin-5-yl)-8-(oxan-4-ylamino)-1-oxo-1,2-dihydro-2,7-naphthyridin-2-yl]acetamide; 2-[6-(2-aminopyrimidin-5-yl)-8-(oxan-4-ylamino)-1-oxo-1,2-dihydro-2,7-naphthyridin-2-yl]acetonitrile; 2-[8-(cyclopentylamino)-1-oxo-6-(pyrazin-2-ylamino)-1,2-dihydro-2,7-naphthyridin-2-yl]acetamide; 2-[8-(cyclopentylamino)-1-oxo-6-(pyrazin-2-ylamino)-1,2-dihydro-2,7-naphthyridin-2-yl]acetonitrile; 2-[8-(cyclobutylamino)-1-oxo-6-(pyrazin-2-ylamino)-1,2-dihydro-2,7-naphthyridin-2-yl]acetamide; 8-(cyclobutylamino)-2-[(3-methyloxetan-3-yl)methyl]-6-(pyrazin-2-ylamino)-1,2-dihydro-2,7-naphthyridin-1-one; 2-[6-(2-aminopyrimidin-5-yl)-8-(cyclopentylamino)-1-oxo-1,2-dihydro-2,7-naphthyridin-2-yl]acetonitrile; 2-[6-(2-aminopyrimidin-5-yl)-8-(cyclopentylamino)-1-oxo-1,2-dihydro-2,7-naphthyridin-2-yl]acetamide; 6-(2-aminopyrimidin-5-yl)-8-[(3,3-difluorocyclobutyl)amino]-2-[(3-methyloxetan-3-yl)methyl]-1,2-dihydro-2,7-naphthyridin-1-one; 2-[6-(2-aminopyrimidin-5-yl)-8-[(3,3-difluorocyclobutyl)amino]-1-oxo-1,2-dihydro-2,7-naphthyridin-2-yl]acetamide; 6-(2-aminopyrimidin-5-yl)-8-(cyclopentylamino)-2-[(3-methyloxetan-3-yl)methyl]-1,2-dihydro-2,7-naphthyridin-1-one; 6-(2-aminopyrimidin-5-yl)-8-(cyclopentylamino)-2-{[3-(hydroxymethyl)oxetan-3-yl]methyl}-1,2-dihydro-2,7-naphthyridin-1-one; 8-(cyclopentylamino)-2-[(3-methyloxetan-3-yl)methyl]-6-(pyrazin-2-ylamino)-1,2-dihydro-2,7-naphthyridin-1-one; 8-(cyclopentylamino)-2-{[3-(hydroxymethyl)oxetan-3-yl]methyl}-6-(pyrazin-2-ylamino)-1,2-dihydro-2,7-naphthyridin-1-one; 8-[(3,3-difluorocyclobutyl)amino]-6-(pyrazin-2-ylamino)-1,2-dihydro-2,7-naphthyridin-1-one; 8-[(3,3-difluorocyclobutyl)amino]-2-[(3-methyloxetan-3-yl)methyl]-6-(pyrazin-2-ylamino)-1,2-dihydro-2,7-naphthyridin-1-one; 8-(cyclopentylamino)-6-(pyrazin-2-ylamino)-1,2-dihydro-2,7-naphthyridin-1-one; 8-(cyclobutylamino)-6-(pyrazin-2-ylamino)-1,2-dihydro-2,7-naphthyridin-1-one; 2-[(3-methyloxetan-3-yl)methyl]-8-(oxan-4-ylamino)-6-(pyrazin-2-ylamino)-1,2-dihydro-2,7-naphthyridin-1-one; 6-(2-aminopyrimidin-5-yl)-2-[(3-methyloxetan-3-yl)methyl]-8-(oxan-4-ylamino)-1,2-dihydro-2,7-naphthyridin-1-one; 6-(2-aminopyrimidin-5-yl)-8-(oxan-4-ylamino)-2-(oxetan-3-yl)-1,2-dihydro-2,7-naphthyridin-1-one; 6-(2-aminopyrimidin-5-yl)-8-(cyclobutylamino)-2-[(3-methyloxetan-3-yl)methyl]-1,2-dihydro-2,7-naphthyridin-1-one; 6-(2-aminopyrimidin-5-yl)-8-(cyclobutylamino)-2-(oxetan-3-yl)-1,2-dihydro-2,7-naphthyridin-1-one; 6-(2-aminopyrimidin-5-yl)-2-[(3-methyloxetan-3-yl)methyl]-8-(oxetan-3-ylamino)-1,2-dihydro-2,7-naphthyridin-1-one; 6-(2-aminopyrimidin-5-yl)-8-(cyclobutylamino)-2-[(2S)-2,3-dihydroxypropyl]-1,2-dihydro-2,7-naphthyridin-1-one; 8-(cyclopentylamino)-2-[(2S)-2,3-dihydroxypropyl]-6-(pyrazin-2-ylamino)-1,2-dihydro-2,7-naphthyridin-1-one; 6-(2-aminopyrimidin-5-yl)-8-(cyclopentylamino)-2-[(2S)-2,3-dihydroxypropyl]-1,2-dihydro-2,7-naphthyridin-1-one; 6-[(5-chloro-4-methylpyridin-2-yl)amino]-8-[(1-hydroxy-2-methylpropan-2-yl)amino]-2-methyl-1,2-dihydro-2,7-naphthyridin-1-one; 2-ethyl-6-[(4-methylpyridin-2-yl)amino]-8-[(3,3,3-trifluoro-2-hydroxypropyl)amino]-1,2-dihydro-2,7-naphthyridin-1-one; 2-ethyl-6-(pyrazin-2-ylamino)-8-[(3,3,3-trifluoro-2-hydroxypropyl)amino]-1,2-dihydro-2,7-naphthyridin-1-one; 2-ethyl-6-[(4-methylpyridin-2-yl)amino]-8-[(3,3,3-trifluoropropyl)amino]-1,2-dihydro-2,7-naphthyridin-1-one; 2-ethyl-6-(pyrazin-2-ylamino)-8-[(3,3,3-trifluoropropyl)amino]-1,2-dihydro-2,7-naphthyridin-1-one; 2-ethyl-8-[(4-hydroxy-2-methylbutan-2-yl)amino]-6-[(4-methylpyridin-2-yl)amino]-1,2-dihydro-2,7-naphthyridin-1-one; 2-ethyl-8-[(4-hydroxy-2-methylbutan-2-yl)amino]-6-(pyrazin-2-ylamino)-1,2-dihydro-2,7-naphthyridin-1-one; 4-chloro-8-[(4-hydroxycyclohexyl)amino]-2-methyl-6-[(4-methylpyridin-2-yl)amino]-1,2-dihydro-2,7-naphthyridin-1-one; 4-chloro-8-[(4-hydroxycyclohexyl)amino]-2-methyl-6-(pyrazin-2-ylamino)-1,2-dihydro-2,7-naphthyridin-1-one; 6-[(4-chloropyridin-2-yl)amino]-8-[(1-hydroxy-2-methylpropan-2-yl)amino]-2-methyl-1,2-dihydro-2,7-naphthyridin-1-one; 2-ethyl-8-{[1-(hydroxymethyl)cyclopentyl]amino}-6-[(4-methylpyridin-2-yl)amino]-1,2-dihydro-2,7-naphthyridin-1-one; 2-ethyl-8-{[1-(hydroxymethyl)cyclopentyl]amino}-6-(pyrazin-2-ylamino)-1,2-dihydro-2,7-naphthyridin-1-one; 2-ethyl-8-{[(1S,3S)-3-(hydroxymethyl)cyclopentyl]amino}-6-[(4-methylpyridin-2-yl)amino]-1,2-dihydro-2,7-naphthyridin-1-one; 2-ethyl-8-{[(1R,3R)-3-(hydroxymethyl)cyclopentyl]amino}-6-[(4-methylpyridin-2-yl)amino]-1,2-dihydro-2,7-naphthyridin-1-one; 2-ethyl-8-{[(1R,3R)-3-(hydroxymethyl)cyclopentyl]amino}-6-(pyrazin-2-ylamino)-1,2-dihydro-2,7-naphthyridin-1-one; 2-ethyl-8-{[(1S,3R)-3-(hydroxymethyl)cyclopentyl]amino}-6-[(4-methylpyridin-2-yl)amino]-1,2-dihydro-2,7-naphthyridin-1-one; 2-ethyl-8-{[(1S,3R)-3-(hydroxymethyl)cyclopentyl]amino}-6-(pyrazin-2-ylamino)-1,2-dihydro-2,7-naphthyridin-1-one; 2-ethyl-8-{[(1R,3S)-3-(hydroxymethyl)cyclopentyl]amino}-6-[(4-methylpyridin-2-yl)amino]-1,2-dihydro-2,7-naphthyridin-1-one; 2-ethyl-8-{[(1R,3S)-3-(hydroxymethyl)cyclopentyl]amino}-6-(pyrazin-2-ylamino)-1,2-dihydro-2,7-naphthyridin-1-one; 2-ethyl-8-{[1-(hydroxymethyl)cyclopropyl]amino}-6-[(4-methylpyridin-2-yl)amino]-1,2-dihydro-2,7-naphthyridin-1-one; 2-ethyl-8-{[1-(hydroxymethyl)cyclopropyl]amino}-6-(pyrazin-2-ylamino)-1,2-dihydro-2,7-naphthyridin-1-one; 8-[(4-hydroxycyclohexyl)amino]-6-[(4-hydroxypyrimidin-2-yl)amino]-2-methyl-1,2-dihydro-2,7-naphthyridin-1-one; 8-[(4-hydroxycyclohexyl)amino]-6-[(4-hydroxypyrimidin-2-yl)amino]-2-methyl-1,2-dihydro-2,7-naphthyridin-1-one; 8-(cyclobutylamino)-6-[(4-hydroxypyrimidin-2-yl)amino]-2-methyl-1,2-dihydro-2,7-naphthyridin-1-one; 8-amino-2-ethyl-6-[(4-methylpyridin-2-yl)amino]-1,2-dihydro-2,7-naphthyridin-1-one; 8-amino-2-ethyl-6-(pyrazin-2-ylamino)-1,2-dihydro-2,7-naphthyridin-1-one; 8-amino-2-ethyl-6-[(4-hydroxypyrimidin-2-yl)amino]-1,2-dihydro-2,7-naphthyridin-1-one; 4-({3-[(4-hydroxypyrimidin-2-yl)amino]-7-methyl-8-oxo-7,8-dihydro-2,7-naphthyridin-1-yl}amino)benzamide; 6-[(4-hydroxypyrimidin-2-yl)amino]-2-methyl-8-({4-[(morpholin-4-yl)carbonyl]phenyl}amino)-1,2-dihydro-2,7-naphthyridin-1-one; 2-ethyl-8-{[(2S)-1-hydroxy-3,3-dimethylbutan-2-yl]amino}-6-[(4-methylpyridin-2-yl)amino]-1,2-dihydro-2,7-naphthyridin-1-one; 2-ethyl-8-{[(2S)-1-hydroxy-3,3-dimethylbutan-2-yl]amino}-6-(pyrazin-2-ylamino)-1,2-dihydro-2,7-naphthyridin-1-one; 8-[(1,3-dihydroxy-2-methylpropan-2-yl)amino]-2-ethyl-6-[(4-methylpyridin-2-yl)amino]-1,2-dihydro-2,7-naphthyridin-1-one; 8-[(1,3-dihydroxy-2-methylpropan-2-yl)amino]-2-ethyl-6-(pyrazin-2-ylamino)-1,2-dihydro-2,7-naphthyridin-1-one; 2-ethyl-8-{[1-(hydroxymethyl)cyclobutyl]amino}-6-[(4-methylpyridin-2-yl)amino]-1,2-dihydro-2,7-naphthyridin-1-one; 8-amino-2-(2-hydroxyethyl)-6-(pyrazin-2-ylamino)-1,2-dihydro-2,7-naphthyridin-1-one; 8-amino-2-(2-hydroxyethyl)-6-[(4-hydroxypyrimidin-2-yl)amino]-1,2-dihydro-2,7-naphthyridin-1-one; 8-amino-2-(2-hydroxyethyl)-6-[(4-methylpyridin-2-yl)amino]-1,2-dihydro-2,7-naphthyridin-1-one; 8-[(2,2-dimethylpropyl)amino]-2-(2-hydroxyethyl)-6-[(4-methylpyridin-2-yl)amino]-1,2-dihydro-2,7-naphthyridin-1-one; 8-[(2,2-difluoroethyl)amino]-2-(2-hydroxyethyl)-6-[(4-methylpyridin-2-yl)amino]-1,2-dihydro-2,7-naphthyridin-1-one; 8-[(2,2-difluoroethyl)amino]-2-(2-hydroxyethyl)-6-(pyrazin-2-ylamino)-1,2-dihydro-2,7-naphthyridin-1-one; 8-(cyclopropylamino)-2-(2-hydroxyethyl)-6-[(4-methylpyridin-2-yl)amino]-1,2-dihydro-2,7-naphthyridin-1-one; 8-(cyclopropylamino)-2-(2-hydroxyethyl)-6-(pyrazin-2-ylamino)-1,2-dihydro-2,7-naphthyridin-1-one; 8-[(1-hydroxy-2-methylpropan-2-yl)amino]-6-[(4-methylpyridin-2-yl)amino]-2-(propan-2-yl)-1,2-dihydro-2,7-naphthyridin-1-one; 8-[(1-hydroxy-2-methylpropan-2-yl)amino]-2-(propan-2-yl)-6-(pyrazin-2-ylamino)-1,2-dihydro-2,7-naphthyridin-1-one; 8-(tert-butylamino)-6-[(4-methylpyridin-2-yl)amino]-2-(propan-2-yl)-1,2-dihydro-2,7-naphthyridin-1-one; 8-(tert-butylamino)-2-(propan-2-yl)-6-(pyrazin-2-ylamino)-1,2-dihydro-2,7-naphthyridin-1-one; 8-(tert-butylamino)-4-chloro-2-(2-hydroxyethyl)-6-(pyrazin-2-ylamino)-1,2-dihydro-2,7-naphthyridin-1-one; 8-(cyclopropylamino)-2-methyl-6-(pyrazin-2-ylamino)-1,2-dihydro-2,7-naphthyridin-1-one; 2-(2-hydroxyethyl)-6-(pyrazin-2-ylamino)-8-[(2,2,2-trifluoroethyl)amino]-1,2-dihydro-2,7-naphthyridin-1-one; 8-(tert-butylamino)-2-(2-hydroxyethyl)-6-[(4-hydroxypyrimidin-2-yl)amino]-1,2-dihydro-2,7-naphthyridin-1-one; 2-ethyl-8-{[(1S,3S)-3-(hydroxymethyl)cyclopentyl]amino}-6-(pyrazin-2-ylamino)-1,2-dihydro-2,7-naphthyridin-1-one; 8-(tert-butylamino)-2-(2-hydroxyethyl)-6-{[4-(pyridin-3-yl)pyridin-2-yl]amino}-1,2-dihydro-2,7-naphthyridin-1-one; 8-(tert-butylamino)-2-(2-hydroxyethyl)-6-[(4-phenylpyridin-2-yl)amino]-1,2-dihydro-2,7-naphthyridin-1-one; 8-(tert-butylamino)-2-(2-hydroxyethyl)-6-{[4-(1-methyl-1H-pyrazol-4-yl)pyridin-2-yl]amino}-1,2-dihydro-2,7-naphthyridin-1-one; 4-{[7-ethyl-8-oxo-3-(pyrazin-2-ylamino)-7,8-dihydro-2,7-naphthyridin-1-yl]amino}-N-(2-hydroxyethyl)benzamide; 2-ethyl-8-({4-[(4-hydroxypiperidin-1-yl)carbonyl]phenyl}amino)-6-(pyrazin-2-ylamino)-1,2-dihydro-2,7-naphthyridin-1-one; 2-ethyl-8-({4-[(3-hydroxypyrrolidin-1-yl)carbonyl]phenyl}amino)-6-(pyrazin-2-ylamino)-1,2-dihydro-2,7-naphthyridin-1-one; 4-({7-ethyl-3-[(4-methylpyridin-2-yl)amino]-8-oxo-7,8-dihydro-2,7-naphthyridin-1-yl}amino)-N-(2-hydroxyethyl)benzamide; 2-ethyl-8-({4-[(4-hydroxypiperidin-1-yl)carbonyl]phenyl}amino)-6-[(4-methylpyridin-2-yl)amino]-1,2-dihydro-2,7-naphthyridin-1-one; 2-ethyl-8-({4-[(3-hydroxypyrrolidin-1-yl)carbonyl]phenyl}amino)-6-[(4-methylpyridin-2-yl)amino]-1,2-dihydro-2,7-naphthyridin-1-one; 8-(tert-butylamino)-5-chloro-2-(2-hydroxyethyl)-6-(pyrazin-2-ylamino)-1,2-dihydro-2,7-naphthyridin-1-one; 2-(2-hydroxyethyl)-8-[(1-methylcyclopropyl)amino]-6-(pyrazin-2-ylamino)-1,2-dihydro-2,7-naphthyridin-1-one; 8-[(1-methylcyclopropyl)amino]-6-(pyrazin-2-ylamino)-1,2-dihydro-2,7-naphthyridin-1-one; 8-(tert-butylamino)-2-(2-hydroxyethyl)-6-{[4-(1H-pyrazol-4-yl)pyridin-2-yl]amino}-1,2-dihydro-2,7-naphthyridin-1-one; 8-(tert-butylamino)-2-(2-hydroxyethyl)-6-{[4-(pyridin-2-yl)pyridin-2-yl]amino}-1,2-dihydro-2,7-naphthyridin-1-one; 8-(tert-butylamino)-2-(2-hydroxyethyl)-6-{[4-(pyridin-4-yl)pyridin-2-yl]amino}-1,2-dihydro-2,7-naphthyridin-1-one; 8-(tert-butylamino)-2-(2-hydroxyethyl)-6-({4-[4-(hydroxymethyl)-1,3-thiazol-2-yl]pyridin-2-yl}amino)-1,2-dihydro-2,7-naphthyridin-1-one; 2-{[1-(tert-butylamino)-7-(2-hydroxyethyl)-8-oxo-7,8-dihydro-2,7-naphthyridin-3-yl]amino}pyridine-4-carbonitrile; 8-(tert-butylamino)-2-(2-hydroxyethyl)-6-({4-[3-(hydroxymethyl)piperidin-1-yl]pyridin-2-yl}amino)-1,2-dihydro-2,7-naphthyridin-1-one; 8-(tert-butylamino)-2-(2-hydroxyethyl)-6-{[4-(4-hydroxypiperidin-1-yl)pyridin-2-yl]amino}-1,2-dihydro-2,7-naphthyridin-1-one; 8-(tert-butylamino)-2-(2-hydroxyethyl)-6-({4-[4-(hydroxymethyl)piperidin-1-yl]pyridin-2-yl}amino)-1,2-dihydro-2,7-naphthyridin-1-one; 8-(tert-butylamino)-2-(2-hydroxyethyl)-6-{[4-(3-hydroxypiperidin-1-yl)pyridin-2-yl]amino}-1,2-dihydro-2,7-naphthyridin-1-one; 2-[4-(2-{[1-(tert-butylamino)-7-(2-hydroxyethyl)-8-oxo-7,8-dihydro-2,7-naphthyridin-3-yl]amino}pyridin-4-yl)-1H-pyrazol-1-yl]acetamide; 8-(tert-butylamino)-2-(2-hydroxyethyl)-6-{[4-(1H-indazol-6-yl)pyridin-2-yl]amino}-1,2-dihydro-2,7-naphthyridin-1-one; 8-(tert-butylamino)-2-(2-hydroxyethyl)-6-{[4-(1H-indazol-5-yl)pyridin-2-yl]amino}-1,2-dihydro-2,7-naphthyridin-1-one; 2-(2-hydroxyethyl)-6-{[4-(4-hydroxypiperidin-1-yl)pyridin-2-yl]amino}-8-(propan-2-ylamino)-1,2-dihydro-2,7-naphthyridin-1-one; 8-(tert-butylamino)-2-(2-hydroxyethyl)-6-{[4-(3-methylpyridin-4-yl)pyridin-2-yl]amino}-1,2-dihydro-2,7-naphthyridin-1-one; 8-(tert-butylamino)-2-(2-hydroxyethyl)-6-{[4-(2-methylpyridin-4-yl)pyridin-2-yl]amino}-1,2-dihydro-2,7-naphthyridin-1-one; 8-(tert-butylamino)-2-(2-hydroxyethyl)-6-{[4-(5-methyl-1H-pyrazol-4-yl)pyridin-2-yl]amino}-1,2-dihydro-2,7-naphthyridin-1-one; 1-(2-{[1-(tert-butylamino)-7-(2-hydroxyethyl)-8-oxo-7,8-dihydro-2,7-naphthyridin-3-yl]amino}pyridin-4-yl)cyclopropane-1-carboxylic acid; 8-(tert-butylamino)-2-(2-hydroxyethyl)-6-{[4-(2-hydroxyethyl)pyridin-2-yl]amino}-1,2-dihydro-2,7-naphthyridin-1-one; 8-(tert-butylamino)-6-[(4-ethenylpyridin-2-yl)amino]-2-(2-hydroxyethyl)-1,2-dihydro-2,7-naphthyridin-1-one; 1-(2-{[1-(tert-butylamino)-7-(2-hydroxyethyl)-8-oxo-7,8-dihydro-2,7-naphthyridin-3-yl]amino}pyridin-4-yl)cyclopropane-1-carboxamide; 8-(tert-butylamino)-2-(2-hydroxyethyl)-6-({4-[1-(hydroxymethyl)cyclopropyl]pyridin-2-yl}amino)-1,2-dihydro-2,7-naphthyridin-1-one; 2-(2-{[1-(tert-butylamino)-7-(2-hydroxyethyl)-8-oxo-7,8-dihydro-2,7-naphthyridin-3-yl]amino}pyridin-4-yl)-2-methylpropanoic acid; 2-(2-{[1-(tert-butylamino)-7-(2-hydroxyethyl)-8-oxo-7,8-dihydro-2,7-naphthyridin-3-yl]amino}pyridin-4-yl)-2-methylpropanamide; 8-(tert-butylamino)-6-{[4-(1-hydroxy-2-methylpropan-2-yl)pyridin-2-yl]amino}-2-(2-hydroxyethyl)-1,2-dihydro-2,7-naphthyridin-1-one; 8-(tert-butylamino)-2-(2-hydroxyethyl)-6-{[4-(1-hydroxypropan-2-yl)pyridin-2-yl]amino}-1,2-dihydro-2,7-naphthyridin-1-one; 2-(2-{[1-(tert-butylamino)-7-(2-hydroxyethyl)-8-oxo-7,8-dihydro-2,7-naphthyridin-3-yl]amino}pyridin-4-yl)-2-methylpropanenitrile; 8-(tert-butylamino)-6-{[4-(2-hydroxyethoxy)pyridin-2-yl]amino}-2-(2-hydroxyethyl)-1,2-dihydro-2,7-naphthyridin-1-one; 8-(tert-butylamino)-6-{[5-fluoro-4-(1-hydroxyethyl)pyridin-2-yl]amino}-2-(2-hydroxyethyl)-1,2-dihydro-2,7-naphthyridin-1-one; 8-(tert-butylamino)-2-(2-hydroxyethyl)-6-{[4-(1-methoxyethyl)pyridin-2-yl]amino}-1,2-dihydro-2,7-naphthyridin-1-one; 2-{[1-(tert-butylamino)-7-(2-hydroxyethyl)-8-oxo-7,8-dihydro-2,7-naphthyridin-3-yl]amino}-N′-hydroxypyridine-4-carboximidamide; 8-(tert-butylamino)-6-{[4-(1-hydroxycyclobutyl)pyridin-2-yl]amino}-2-(2-hydroxyethyl)-1,2-dihydro-2,7-naphthyridin-1-one; 8-(tert-butylamino)-6-{[4-(1,1-difluoro-2-hydroxyethyl)pyridin-2-yl]amino}-2-(2-hydroxyethyl)-1,2-dihydro-2,7-naphthyridin-1-one; 8-(tert-butylamino)-6-{[4-(1-fluoro-2-hydroxyethyl)pyridin-2-yl]amino}-2-(2-hydroxyethyl)-1,2-dihydro-2,7-naphthyridin-1-one; 8-(tert-butylamino)-2-(2-hydroxyethyl)-6-{[4-(5-methyl-1,2,4-oxadiazol-3-yl)pyridin-2-yl]amino}-1,2-dihydro-2,7-naphthyridin-1-one; 8-(tert-butylamino)-6-{[4-(1,1-difluoroethyl)pyridin-2-yl]amino}-2-(2-hydroxyethyl)-1,2-dihydro-2,7-naphthyridin-1-one; 2-[8-(tert-butylamino)-6-{[4-(hydroxymethyl)pyridin-2-yl]amino}-1-oxo-1,2-dihydro-2,7-naphthyridin-2-yl]acetic acid; 8-(tert-butylamino)-6-{[5-fluoro-4-(1-hydroxyethyl)pyridin-2-yl]amino}-2-(2-hydroxyethyl)-1,2-dihydro-2,7-naphthyridin-1-one; 2-[8-(tert-butylamino)-6-{[5-fluoro-4-(2-hydroxypropan-2-yl)pyridin-2-yl]amino}-1-oxo-1,2-dihydro-2,7-naphthyridin-2-yl]acetamide; 8-(tert-butylamino)-2-(2-hydroxyethyl)-6-{[4-(pyrimidin-2-yl)pyridin-2-yl]amino}-1,2-dihydro-2,7-naphthyridin-1-one; 2-{[1-(tert-butylamino)-7-(2-hydroxyethyl)-8-oxo-7,8-dihydro-2,7-naphthyridin-3-yl]amino}-N-hydroxypyridine-4-carboxamide; 8-(tert-butylamino)-6-{[4-(2-hydroxy-2-methylpropyl)pyridin-2-yl]amino}-2-(2-hydroxyethyl)-1,2-dihydro-2,7-naphthyridin-1-one; 8-(tert-butylamino)-6-(pyrazin-2-ylamino)-2-(1H-1,2,3-triazol-5-ylmethyl)-1,2-dihydro-2,7-naphthyridin-1-one; 8-(tert-butylamino)-2-(prop-2-yn-1-yl)-6-(pyrazin-2-ylamino)-1,2-dihydro-2,7-naphthyridin-1-one; 8-(tert-butylamino)-2-(2-hydroxy-2-methylpropyl)-6-(pyrazin-2-ylamino)-1,2-dihydro-2,7-naphthyridin-1-one; 8-(tert-butylamino)-6-(pyrazin-2-ylamino)-2-(1H-1,2,3,4-tetrazol-5-ylmethyl)-1,2-dihydro-2,7-naphthyridin-1-one; 8-(tert-butylamino)-2-(2,3-dihydroxypropyl)-6-{[6-(2-methoxyethoxy)pyrimidin-4-yl]amino}-1,2-dihydro-2,7-naphthyridin-1-one; 8-(tert-butylamino)-2-(2-hydroxyethyl)-6-{[6-methylpyrimidin-4-yl)amino]-1,2-dihydro-2,7-naphthyridin-1-one; 8-(tert-butylamino)-2-(2,3-dihydroxypropyl)-6-[6-(oxolan-3-ylmethoxy)pyrimidin-4-yl]amino}-1,2-dihydro-2,7-naphthyridin-1-one; 1-(6-{[1-(tert-butylamino)-7-(2-hydroxyethyl)-8-oxo-7,8-dihydro-2,7-naphthyridin-3-yl]amino}pyrimidin-4-yl)-1H-pyrazole-4-carboxylic acid; ethyl 1-(6-{[1-(tert-butylamino)-7-(2-hydroxyethyl)-8-oxo-7,8-dihydro-2,7-naphthyridin-3-yl]amino}pyrimidin-4-yl)-1H-pyrazole-4-carboxylate; 8-(tert-butylamino)-2-[(2S)-2,3-dihydroxypropyl]-6-{[6-(1H-pyrazol-4-yloxy)pyrimidin-4-yl]amino}-1,2-dihydro-2,7-naphthyridin-1-one; 8-(tert-butylamino)-2-(2,3-dihydroxypropyl)-6-{[6-(oxolan-2-ylmethoxy)pyrimidin-4-yl]amino}-1,2-dihydro-2,7-naphthyridin-1-one; 6-(2-aminopyrimidin-5-yl)-2-(2-hydroxyethyl)-8-(propan-2-ylamino)-1,2-dihydro-2,7-naphthyridin-1-one; 6-(2-aminopyrimidin-5-yl)-8-[(2-methylbutan-2-yl)amino]-1,2-dihydro-2,7-naphthyridin-1-one; 6-(2-aminopyrimidin-5-yl)-8-(tert-butylamino)-1,2-dihydro-2,7-naphthyridin-1-one; 2-ethyl-8-[(1-hydroxy-2-methylpropan-2-yl)amino]-6-(pyrazin-2-ylamino)-1,2-dihydro-2,7-naphthyridin-1-one; 2-(2-hydroxyethyl)-8-[(1-methylcyclobutyl)amino]-6-(pyrazin-2-ylamino)-1,2-dihydro-2,7-naphthyridin-1-one; 2-ethyl-8-{[1-(hydroxymethyl)cyclobutyl]amino}-6-(pyrazin-2-ylamino)-1,2-dihydro-2,7-naphthyridin-1-one; 8-[(2,2-dimethylpropyl)amino]-2-(2-hydroxyethyl)-6-(pyrazin-2-ylamino)-1,2-dihydro-2,7-naphthyridin-1-one; 6-(2-aminopyrimidin-5-yl)-8-(tert-butylamino)-2-(2-hydroxyethyl)-1,2-dihydro-2,7-naphthyridin-1-one; 6-(6-aminopyridin-3-yl)-8-(tert-butylamino)-2-(2-hydroxyethyl)-1,2-dihydro-2,7-naphthyridin-1-one; 8-(tert-butylamino)-2-(2-hydroxyethyl)-6-{[4-(trifluoromethyl)pyridin-2-yl]amino}-1,2-dihydro-2,7-naphthyridin-1-one; 6-(2-aminopyrimidin-5-yl)-8-[(1-hydroxy-2-methylpropan-2-yl)amino]-2-(2-hydroxyethyl)-1,2-dihydro-2,7-naphthyridin-1-one; 6-(2-aminopyrimidin-5-yl)-2-(2-hydroxyethyl)-8-{[1-(hydroxymethyl)cyclobutyl]amino}-1,2-dihydro-2,7-naphthyridin-1-one; 6-(2-aminopyrimidin-5-yl)-2-ethyl-8-([(1-hydroxy-2-methylpropan-2-yl)amino]-1,2-dihydro-2,7-naphthyridin-1-one; 6-(2-aminopyrimidin-5-yl)-2-ethyl-8-{[1-(hydroxymethyl)cyclobutyl]amino}-1,2-dihydro-2,7-naphthyridin-1-one; 6-(2-aminopyrimidin-5-yl)-2-ethyl-8-[(1-methylcyclobutyl)amino]-1,2-dihydro-2,7-naphthyridin-1-one; 6-(2-aminopyrimidin-5-yl)-2-(2-hydroxyethyl)-8-[(1-methylcyclobutyl)amino]-1,2-dihydro-2,7-naphthyridin-1-one; 6-(2-aminopyrimidin-5-yl)-2-(2-hydroxyethyl)-8-[(2-methylbutan-2-yl)amino]-1,2-dihydro-2,7-naphthyridin-1-one; 6-(2-aminopyrimidin-5-yl)-2-(2-hydroxyethyl)-8-(pyrrolidin-1-yl)-1,2-dihydro-2,7-naphthyridin-1-one; 6-(2-aminopyrimidin-5-yl)-8-(tert-butylamino)-2-(2-methoxyethyl)-1,2-dihydro-2,7-naphthyridin-1-one; 6-(2-aminopyrimidin-5-yl)-8-[(2,2-dimethylpropyl)amino]-2-(2-hydroxyethyl)-1,2-dihydro-2,7-naphthyridin-1-one; 6-(2-aminopyrimidin-5-yl)-2-(2-hydroxyethyl)-8-{[(1S,3R)-3-(hydroxymethyl)cyclopentyl]amino}-1,2-dihydro-2,7-naphthyridin-1-one; 6-(2-aminopyrimidin-5-yl)-2-ethyl-8-{[(1S,3R)-3-(hydroxymethyl)cyclopentyl]amino}-1,2-dihydro-2,7-naphthyridin-1-one; 6-(2-aminopyrimidin-5-yl)-8-(tert-butylamino)-2-propyl-1,2-dihydro-2,7-naphthyridin-1-one; 6-(2-aminopyrimidin-5-yl)-8-(tert-butylamino)-2-(3-hydroxypropyl)-1,2-dihydro-2,7-naphthyridin-1-one; 8-(tert-butylamino)-2-(2-hydroxyethyl)-6-[2-(methylamino)pyrimidin-5-yl]-1,2-dihydro-2,7-naphthyridin-1-one; 2-(2-hydroxyethyl)-8-[(2-methylbutan-2-yl)amino]-6-(pyrazin-2-ylamino)-1,2-dihydro-2,7-naphthyridin-1-one; 6-(2-aminopyrimidin-5-yl)-8-(tert-butylamino)-2-(oxolan-2-ylmethyl)-1,2-dihydro-2,7-naphthyridin-1-one; 6-(2-aminopyrimidin-5-yl)-8-(tert-butylamino)-2-(oxan-4-ylmethyl)-1,2-dihydro-2,7-naphthyridin-1-one; 6-(2-aminopyrimidin-5-yl)-8-(tert-butylamino)-2-(4-hydroxybutyl)-1,2-dihydro-2,7-naphthyridin-1-one; 6-(2-aminopyrimidin-5-yl)-8-(tert-butylamino)-2-(oxetan-3-yl)-1,2-dihydro-2,7-naphthyridin-1-one; 6-(2-aminopyrimidin-5-yl)-8-(tert-butylamino)-2-(1,3-dihydroxypropan-2-yl)-1,2-dihydro-2,7-naphthyridin-1-one; 6-(2-aminopyrimidin-5-yl)-8-(tert-butylamino)-2-{[3-(hydroxymethyl)oxetan-3-yl]methyl}-1,2-dihydro-2,7-naphthyridin-1-one; 6-(2-aminopyrimidin-5-yl)-8-(tert-butylamino)-2-[(3-methyloxetan-3-yl)methyl]-1,2-dihydro-2,7-naphthyridin-1-one; 6-(2-aminopyrimidin-5-yl)-8-{[2-(4-fluorophenyl)propan-2-yl]amino}-2-(2-hydroxyethyl)-1,2-dihydro-2,7-naphthyridin-1-one; 6-(2-aminopyrimidin-5-yl)-8-(tert-butylamino)-2-(3,3,3-trifluoro-2-hydroxypropyl)-1,2-dihydro-2,7-naphthyridin-1-one; 6-(2-aminopyrimidin-5-yl)-8-(tert-butylamino)-2-(2-hydroxypropyl)-1,2-dihydro-2,7-naphthyridin-1-one; 6-(2-aminopyrimidin-5-yl)-8-(tert-butylamino)-2-(2-hydroxy-2-methylpropyl)-1,2-dihydro-2,7-naphthyridin-1-one; 6-(2-aminopyrimidin-5-yl)-8-(tert-butylamino)-2-(2,3-dihydroxypropyl)-1,2-dihydro-2,7-naphthyridin-1-one; 6-(2-aminopyrimidin-5-yl)-8-[(1-methylcyclobutyl)amino]-2-(oxan-4-ylmethyl)-1,2-dihydro-2,7-naphthyridin-1-one; 6-(2-aminopyrimidin-5-yl)-8-[(1-methylcyclobutyl)amino]-2-(oxetan-3-yl)-1,2-dihydro-2,7-naphthyridin-1-one; 6-(2-aminopyrimidin-5-yl)-8-[(1-methylcyclobutyl)amino]-2-[(3-methyloxetan-3-yl)methyl]-1,2-dihydro-2,7-naphthyridin-1-one; 6-(2-aminopyrimidin-5-yl)-2-{[3-(hydroxymethyl)oxetan-3-yl]methyl}-8-[(1-methylcyclobutyl)amino]-1,2-dihydro-2,7-naphthyridin-1-one; 6-(2-aminopyrimidin-5-yl)-8-(tert-butylamino)-2-[(2S)-2,3-dihydroxypropyl]-1,2-dihydro-2,7-naphthyridin-1-one; 6-(2-aminopyrimidin-5-yl)-8-(tert-butylamino)-2-[(2R)-2,3-dihydroxypropyl]-1,2-dihydro-2,7-naphthyridin-1-one; 6-(2-aminopyrimidin-5-yl)-8-(tert-butylamino)-2-(1,3-dimethoxypropan-2-yl)-1,2-dihydro-2,7-naphthyridin-1-one; 8-(tert-butylamino)-2-(1,3-dimethoxypropan-2-yl)-6-(pyrazin-2-ylamino)-1,2-dihydro-2,7-naphthyridin-1-one; 2-ethyl-8-{[(1S,2R)-2-hydroxycyclopentyl]amino}-6-(pyrazin-2-ylamino)-1,2-dihydro-2,7-naphthyridin-1-one; 2-ethyl-8-{[(1R,2R)-2-hydroxycyclopentyl]amino}-6-[(4-methylpyridin-2-yl)amino]-1,2-dihydro-2,7-naphthyridin-1-one; 2-ethyl-8-{[(1R,2R)-2-hydroxycyclopentyl]amino}-6-(pyrazin-2-ylamino)-1,2-dihydro-2,7-naphthyridin-1-one; 2-ethyl-8-{[(1R,2S)-2-(hydroxymethyl)cyclopentyl]amino}-6-[(4-methylpyridin-2-yl)amino]-1,2-dihydro-2,7-naphthyridin-1-one; 2-ethyl-8-{[(1R,2S)-2-(hydroxymethyl)cyclopentyl]amino}-6-(pyrazin-2-ylamino)-1,2-dihydro-2,7-naphthyridin-1-one; 8-[(4-hydroxycyclohexyl)amino]-6-[(4-methylpyridin-2-yl)amino]-2-(propan-2-yl)-1,2-dihydro-2,7-naphthyridin-1-one; 8-[(4-hydroxycyclohexyl)amino]-2-(propan-2-yl)-6-(pyrazin-2-ylamino)-1,2-dihydro-2,7-naphthyridin-1-one; 2-ethyl-8-{[(1S,3S)-3-hydroxycyclopentyl]amino}-6-[(4-methylpyridin-2-yl)amino]-1,2-dihydro-2,7-naphthyridin-1-one; 2-ethyl-8-{[(1S,3S)-3-hydroxycyclopentyl]amino}-6-(pyrazin-2-ylamino)-1,2-dihydro-2,7-naphthyridin-1-one; 8-[(4-hydroxycyclohexyl)amino]-2-(3-hydroxypropyl)-6-[(4-methylpyridin-2-yl)amino]-1,2-dihydro-2,7-naphthyridin-1-one; 8-[(4-hydroxycyclohexyl)amino]-2-(3-hydroxypropyl)-6-(pyrazin-2-ylamino)-1,2-dihydro-2,7-naphthyridin-1-one; 2-ethyl-8-[(4-hydroxycyclohexyl)amino]-6-(pyrazin-2-ylamino)-1,2-dihydro-2,7-naphthyridin-1-one; 2-ethyl-8-[(4-hydroxy-4-methylcyclohexyl)amino]-6-[(4-methylpyridin-2-yl)amino]-1,2-dihydro-2,7-naphthyridin-1-one; 2-ethyl-8-[(4-hydroxy-4-methylcyclohexyl)amino]-6-[(4-methylpyridin-2-yl)amino]-1,2-dihydro-2,7-naphthyridin-1-one; 2-ethyl-8-[(4-hydroxy-4-methylcyclohexyl)amino]-6-(pyrazin-2-ylamino)-1,2-dihydro-2,7-naphthyridin-1-one; 2-ethyl-8-[(4-hydroxy-4-methylcyclohexyl)amino]-6-(pyrazin-2-ylamino)-1,2-dihydro-2,7-naphthyridin-1-one; 2-ethyl-8-[(4-methylcyclohex-3-en-1-yl)amino]-6-[(4-methylpyridin-2-yl)amino]-1,2-dihydro-2,7-naphthyridin-1-one; 2-ethyl-8-[(4-methylcyclohex-3-en-1-yl)amino]-6-(pyrazin-2-ylamino)-1,2-dihydro-2,7-naphthyridin-1-one; 2-ethyl-8-{[(1S,2S)-2-hydroxycyclopentyl]amino}-6-[(4-hydroxypyrimidin-2-yl)amino]-1,2-dihydro-2,7-naphthyridin-1-one; 2-ethyl-8-{[(1S,2R)-2-hydroxycyclopentyl]amino}-6-[(4-hydroxypyrimidin-2-yl)amino]-1,2-dihydro-2,7-naphthyridin-1-one; 2-ethyl-8-{[(1R,3S)-3-hydroxycyclopentyl]amino}-6-[(4-hydroxypyrimidin-2-yl)amino]-1,2-dihydro-2,7-naphthyridin-1-one; 2-ethyl-8-({[(1R,3R)-3-hydroxycyclopentyl]methyl}amino)-6-[(4-hydroxypyrimidin-2-yl)amino]-1,2-dihydro-2,7-naphthyridin-1-one; 2-ethyl-8-{[(1R,3R)-3-hydroxycyclopentyl]amino}-6-[(4-hydroxypyrimidin-2-yl)amino]-1,2-dihydro-2,7-naphthyridin-1-one; 2-ethyl-8-{[(1S,3R)-3-hydroxycyclopentyl]amino}-6-[(4-hydroxypyrimidin-2-yl)amino]-1,2-dihydro-2,7-naphthyridin-1-one; 2-ethyl-8-{[(2R)-1-hydroxypropan-2-yl]amino}-6-[(4-methylpyridin-2-yl)amino]-1,2-dihydro-2,7-naphthyridin-1-one; 2-ethyl-8-{[(2R)-1-hydroxypropan-2-yl]amino}-6-(pyrazin-2-ylamino)-1,2-dihydro-2,7-naphthyridin-1-one; 8-[(4-hydroxycyclohexyl)amino]-6-[(4-methylpyridin-2-yl)amino]-2-(propan-2-yl)-1,2-dihydro-2,7-naphthyridin-1-one; 8-[(4-hydroxycyclohexyl)amino]-2-(propan-2-yl)-6-(pyrazin-2-ylamino)-1,2-dihydro-2,7-naphthyridin-1-one; 8-{[(2S)-1-hydroxypropan-2-yl]amino}-6-[(4-methylpyridin-2-yl)amino]-2-(propan-2-yl)-1,2-dihydro-2,7-naphthyridin-1-one; 8-{[(2S)-1-hydroxypropan-2-yl]amino}-2-(propan-2-yl)-6-(pyrazin-2-ylamino)-1,2-dihydro-2,7-naphthyridin-1-one; 2-ethyl-6-[(4-methylpyridin-2-yl)amino]-8-{[(3R)-6-oxopiperidin-3-yl]amino}-1,2-dihydro-2,7-naphthyridin-1-one; 2-ethyl-8-{[(3R)-6-oxopiperidin-3-yl]amino}-6-(pyrazin-2-ylamino)-1,2-dihydro-2,7-naphthyridin-1-one; 3-({7-ethyl-3-[(4-methylpyridin-2-yl)amino]-8-oxo-7,8-dihydro-2,7-naphthyridin-1-yl}amino)-1$1̂{6}-thiolane-1,1-dione; 3-{[7-ethyl-8-oxo-3-(pyrazin-2-ylamino)-7,8-dihydro-2,7-naphthyridin-1-yl]amino}-1$1̂{6}-thiolane-1,1-dione; 2-ethyl-8-[(3-hydroxycyclohexyl)amino]-6-[(4-methylpyridin-2-yl)amino]-1,2-dihydro-2,7-naphthyridin-1-one; 2-ethyl-8-[(3-hydroxycyclohexyl)amino]-6-(pyrazin-2-ylamino)-1,2-dihydro-2,7-naphthyridin-1-one; 3-{[7-(2-hydroxyethyl)-3-[(4-methylpyridin-2-yl)amino]-8-oxo-7,8-dihydro-2,7-naphthyridin-1-yl]amino}-1$1̂{6}-thiolane-1,1-dione; 3-{[7-(2-hydroxyethyl)-8-oxo-3-(pyrazin-2-ylamino)-7,8-dihydro-2,7-naphthyridin-1-yl]amino}-1$1̂{6}-thiolane-1,1-dione; 8-[(4-hydroxycyclohexyl)amino]-6-(pyrazin-2-ylamino)-1,2-dihydro-2,7-naphthyridin-1-one; 8-{[(1S,3R)-3-hydroxycyclopentyl]amino}-6-[(4-methylpyridin-2-yl)amino]-2-(propan-2-yl)-1,2-dihydro-2,7-naphthyridin-1-one; 8-{[(1S,3R)-3-hydroxycyclopentyl]amino}-2-(propan-2-yl)-6-(pyrazin-2-ylamino)-1,2-dihydro-2,7-naphthyridin-1-one; 8-{[(1S,2R)-2-hydroxycyclopentyl]amino}-6-[(4-methylpyridin-2-yl)amino]-2-(propan-2-yl)-1,2-dihydro-2,7-naphthyridin-1-one; 8-{[(1S,2R)-2-hydroxycyclopentyl]amino}-2-(propan-2-yl)-6-(pyrazin-2-ylamino)-1,2-dihydro-2,7-naphthyridin-1-one; 8-[(4-hydroxycyclohexyl)amino]-6-(pyrazin-2-ylamino)-1,2-dihydro-2,7-naphthyridin-1-one; 8-[(4-hydroxycyclohexyl)amino]-2-(2-methanesulfonylethyl)-6-(pyrazin-2-ylamino)-1,2-dihydro-2,7-naphthyridin-1-one; 8-[(4-hydroxycyclohexyl)amino]-2-methyl-6-[(4-methylpyridin-2-yl)amino]-1,2-dihydro-2,7-naphthyridin-1-one; 8-[(4-hydroxycyclohexyl)amino]-2-methyl-6-(pyrazin-2-ylamino)-1,2-dihydro-2,7-naphthyridin-1-one; 8-{[(1S,3R)-3-(hydroxymethyl)cyclopentyl]amino}-2-methyl-6-[(4-methylpyridin-2-yl)amino]-1,2-dihydro-2,7-naphthyridin-1-one; 8-{[(1S,3R)-3-(hydroxymethyl)cyclopentyl]amino}-2-methyl-6-(pyrazin-2-ylamino)-1,2-dihydro-2,7-naphthyridin-1-one; 8-{[(1S,3R)-3-(hydroxymethyl)cyclopentyl]amino}-6-[(4-methylpyridin-2-yl)amino]-2-(propan-2-yl)-1,2-dihydro-2,7-naphthyridin-1-one; 8-{[(1S,3R)-3-(hydroxymethyl)cyclopentyl]amino}-2-(propan-2-yl)-6-(pyrazin-2-ylamino)-1,2-dihydro-2,7-naphthyridin-1-one; 8-[(4-hydroxycyclohexyl)amino]-2-(2-hydroxyethyl)-6-(pyrazin-2-ylamino)-1,2-dihydro-2,7-naphthyridin-1-one; 2-ethyl-8-[(4-hydrogenio-4-hydroxycyclohexyl)amino]-6-[(4-methylpyridin-2-yl)amino]-1,2-dihydro-2,7-naphthyridin-1-one; 2-ethyl-8-[(4-hydrogenio-4-hydroxycyclohexyl)amino]-6-(pyrazin-2-ylamino)-1,2-dihydro-2,7-naphthyridin-1-one; 2-ethyl-8-[(4-hydrogenio-4-hydroxycyclohexyl)amino]-6-(pyrazin-2-ylamino)-1,2-dihydro-2,7-naphthyridin-1-one; 8-[(4-hydroxycyclohexyl)amino]-6-[(4-methylpyridin-2-yl)amino]-2-[2-(morpholin-4-yl)ethyl]-1,2-dihydro-2,7-naphthyridin-1-one; 8-[(4-hydroxycyclohexyl)amino]-2-[2-(morpholin-4-yl)ethyl]-6-(pyrazin-2-ylamino)-1,2-dihydro-2,7-naphthyridin-1-one; 8-{[(2S)-1-hydroxypropan-2-yl]amino}-6-[(4-methylpyridin-2-yl)amino]-2-[2-(morpholin-4-yl)ethyl]-1,2-dihydro-2,7-naphthyridin-1-one; 8-{[(2S)-1-hydroxypropan-2-yl]amino}-2-[2-(morpholin-4-yl)ethyl]-6-(pyrazin-2-ylamino)-1,2-dihydro-2,7-naphthyridin-1-one; 8-(tert-butylamino)-6-[(4-methylpyridin-2-yl)amino]-2-[2-(morpholin-4-yl)ethyl]-1,2-dihydro-2,7-naphthyridin-1-one; 8-(tert-butylamino)-2-[2-(morpholin-4-yl)ethyl]-6-(pyrazin-2-ylamino)-1,2-dihydro-2,7-naphthyridin-1-one; 2-(2-fluoroethyl)-8-[(4-hydroxycyclohexyl)amino]-6-(pyrazin-2-ylamino)-1,2-dihydro-2,7-naphthyridin-1-one; 2-(2-fluoroethyl)-8-{[(2S)-1-hydroxypropan-2-yl]amino}-6-(pyrazin-2-ylamino)-1,2-dihydro-2,7-naphthyridin-1-one; 8-(tert-butylamino)-2-(2-fluoroethyl)-6-(pyrazin-2-ylamino)-1,2-dihydro-2,7-naphthyridin-1-one; 3-{8-[(4-hydroxycyclohexyl)amino}-1-oxo-6-(pyrazin-2-ylamino)-1,2-dihydro-2,7-naphthyridin-2-yl]propanenitrile; 3-(8-{[(2S)-1-hydroxypropan-2-yl]amino}-1-oxo-6-(pyrazin-2-ylamino)-1,2-dihydro-2,7-naphthyridin-2-yl)propanenitrile; 3-[8-(tert-butylamino)-1-oxo-6-(pyrazin-2-ylamino)-1,2-dihydro-2,7-naphthyridin-2-yl]propanenitrile; 8-[(4-hydroxycyclohexyl)amino]-2-(2-methoxyethyl)-6-(pyrazin-2-ylamino)-1,2-dihydro-2,7-naphthyridin-1-one; 8-(tert-butylamino)-2-(2-methoxyethyl)-6-(pyrazin-2-ylamino)-1,2-dihydro-2,7-naphthyridin-1-one; 8-[(4-hydroxycyclohexyl)amino]-2-[2-(morpholin-4-yl)-2-oxoethyl]-6-(pyrazin-2-ylamino)-1,2-dihydro-2,7-naphthyridin-1-one; 8-(tert-butylamino)-2-[2-(morpholin-4-yl)-2-oxoethyl]-6-(pyrazin-2-ylamino)-1,2-dihydro-2,7-naphthyridin-1-one; 8-[(4-hydroxycyclohexyl)amino]-2-(oxetan-3-yl)-6-(pyrazin-2-ylamino)-1,2-dihydro-2,7-naphthyridin-1-one; 3-{8-[(4-hydroxycyclohexyl)amino]-1-oxo-6-(pyrazin-2-ylamino)-1,2-dihydro-2,7-naphthyridin-2-yl}-1$1̂{6}-thietane-1,1-dione; 8-(tert-butylamino)-6-(pyrazin-2-ylamino)-2-(3,3,3-trifluoro-2-hydroxypropyl)-1,2-dihydro-2,7-naphthyridin-1-one; 8-[(4-hydroxycyclohexyl)amino]-6-(pyrazin-2-ylamino)-2-(3,3,3-trifluoro-2-hydroxypropyl)-1,2-dihydro-2,7-naphthyridin-1-one; 8-(tert-butylamino)-2-[2-(2-hydroxyethoxy)ethyl]-6-(pyrazin-2-ylamino)-1,2-dihydro-2,7-naphthyridin-1-one; 8-(tert-butylamino)-2-(oxetan-3-yl)-6-(pyrazin-2-ylamino)-1,2-dihydro-2,7-naphthyridin-1-one; 3-[8-(tert-butylamino)-1-oxo-6-(pyrazin-2-ylamino)-1,2-dihydro-2,7-naphthyridin-2-yl]-1$1̂{6}-thietane-1,1-dione; 3-({1-[(4-hydroxycyclohexyl)amino]-8-oxo-7-(propan-2-yl)-7,8-dihydro-2,7-naphthyridin-3-yl}amino)pyrazin-1-ium-1-olate; 3-[(1-{[(1S,3R)-3-hydroxycyclopentyl]amino}-8-oxo-7-(propan-2-yl)-7,8-dihydro-2,7-naphthyridin-3-yl)amino]pyrazin-1-ium-1-olate; 3-[(1-{[(1S,3R)-3-(hydroxymethyl)cyclopentyl]amino}-8-oxo-7-(propan-2-yl)-7,8-dihydro-2,7-naphthyridin-3-yl)amino]pyrazin-1-ium-1-olate; 8-(tert-butylamino)-6-(pyrazin-2-ylamino)-2-(pyridin-4-ylmethyl)-1,2-dihydro-2,7-naphthyridin-1-one; 8-(tert-butylamino)-6-(pyrazin-2-ylamino)-2-(pyridin-3-ylmethyl)-1,2-dihydro-2,7-naphthyridin-1-one; 8-(tert-butylamino)-6-(pyrazin-2-ylamino)-2-(pyridin-2-ylmethyl)-1,2-dihydro-2,7-naphthyridin-1-one; 2-(2,2-difluoroethyl)-8-[(4-hydroxycyclohexyl)amino]-6-(pyrazin-2-ylamino)-1,2-dihydro-2,7-naphthyridin-1-one; 8-(tert-butylamino)-2-(2,2-difluoroethyl)-6-(pyrazin-2-ylamino)-1,2-dihydro-2,7-naphthyridin-1-one; 8-[(4-hydroxycyclohexyl)amino]-6-(pyrazin-2-ylamino)-2-(pyridin-4-ylmethyl)-1,2-dihydro-2,7-naphthyridin-1-one; 8-[(4-hydroxycyclohexyl)amino]-6-(pyrazin-2-ylamino)-2-(2,2,2-trifluoroethyl)-1,2-dihydro-2,7-naphthyridin-1-one; 6-(2-aminopyrimidin-5-yl)-8-[(4-hydroxycyclohexyl)amino]-2-methyl-1,2-dihydro-2,7-naphthyridin-1-one; 6-(2-aminopyrimidin-5-yl)-8-[(4-hydroxycyclohexyl)amino]-2-(propan-2-yl)-1,2-dihydro-2,7-naphthyridin-1-one; 6-(2-aminopyrimidin-5-yl)-8-[(4-hydroxycyclohexyl)amino]-2-(2-hydroxyethyl)-1,2-dihydro-2,7-naphthyridin-1-one; 6-(2-aminopyrimidin-5-yl)-8-[(4-hydroxycyclohexyl)amino]-2-[2-(morpholin-4-yl)ethyl]-1,2-dihydro-2,7-naphthyridin-1-one; 6-(2-aminopyrimidin-5-yl)-8-{[(1S,3R)-3-(hydroxymethyl)cyclopentyl]amino}-2-(propan-2-yl)-1,2-dihydro-2,7-naphthyridin-1-one; 6-(2-aminopyrimidin-5-yl)-8-{[(1S,3R)-3-hydroxycyclopentyl]amino}-2-(propan-2-yl)-1,2-dihydro-2,7-naphthyridin-1-one; 6-(2-aminopyrimidin-5-yl)-2-ethyl-8-[(4-hydroxycyclohexyl)amino]-1,2-dihydro-2,7-naphthyridin-1-one; 6-(2-aminopyrimidin-5-yl)-8-(tert-butylamino)-2-[2-(morpholin-4-yl)ethyl]-1,2-dihydro-2,7-naphthyridin-1-one; 8-(tert-butylamino)-2-(3-hydroxypropyl)-6-(pyrazin-2-ylamino)-1,2-dihydro-2,7-naphthyridin-1-one; 6-(2-aminopyrimidin-5-yl)-8-(cyclopropylamino)-2-(2-hydroxyethyl)-1,2-dihydro-2,7-naphthyridin-1-one; 6-(2-aminopyrimidin-5-yl)-2-(2-hydroxyethyl)-8-[(1-methylcyclopropyl)amino]-1,2-dihydro-2,7-naphthyridin-1-one; 6-(2-aminopyrimidin-5-yl)-2-ethyl-8-[(4-hydroxy-2-methylbutan-2-yl)amino]-1,2-dihydro-2,7-naphthyridin-1-one; 6-(2-aminopyrimidin-5-yl)-2-ethyl-8-{[1-(hydroxymethyl)cyclopropyl]amino}-1,2-dihydro-2,7-naphthyridin-1-one, and 6-(2-aminopyrimidin-5-yl)-2-ethyl-8-{[(1S,3R)-3-hydroxycyclopentyl]amino}-1,2-dihydro-2,7-naphthyridin-1-one.

Another aspect provided herein are pharmaceutical compositions for treating a Syk kinase mediated disease comprising a therapeutically effective amount of any aforementioned compound of Formula (I) and a pharmaceutically acceptable excipient.

Another aspect provided herein are medicaments for treating a Syk kinase mediated disease, wherein the medicament comprises a therapeutically effective amount of any aforementioned compound of Formula (I).

Another aspect provided herein is the use of any one of the aforementioned compounds of a Formula (I) in the manufacture of a medicament for treating a Syk-mediated disease in a subject in need thereof.

Another aspect provided herein is a method for inhibiting a Syk kinase, comprising administering to a system or a subject in need thereof a therapeutically effective amount of any one of the aforementioned compounds of Formula (I).

Another aspect provided herein is a method for treating a Syk-mediated disease comprising administering to a subject in need thereof a therapeutically effective amount of any one of the aforementioned compounds of Formula (I).

In certain embodiments of such aspects the Syk kinase mediated disease is an inflammatory disease, an allergic disease, a cell-proliferative disease, an autoimmune disease or cytopenia.

In certain embodiments of such aspects the Syk kinase mediated disease is allergic asthma, allergic rhinitis, rheumatoid arthritis, multiple sclerosis, lupus, systemic lupus erythematosus, lymphoma, B cell lymphoma, T cell lymphoma, myelodysplasic syndrome, anemia, leucopenia, neutropenia, thrombocytopenia, granuloctopenia, pancytoia or idiopathic thrombocytopenic purpura.

Another aspect provided herein is a compound for use in a method of medical treatment, wherein the method of medical treatment is for treating a Syk kinase mediated disease, wherein the disease is selected from allergic asthma, allergic rhinitis, rheumatoid arthritis, multiple sclerosis, lupus, systemic lupus erythematosus, lymphoma, B cell lymphoma, T cell lymphoma, myelodysplasic syndrome, anemia, leucopenia, neutropenia, thrombocytopenia, granuloctopenia, pancytoia and idiopathic thrombocytopenic purpura, and wherein the compound is any one of the aforementioned compounds of Formula (I).

### DETAILED DESCRIPTION OF THE INVENTION

### Definitions

The terms “alkenyl” or “alkene”, as used herein, refers to a partially unsaturated branched or straight chain hydrocarbon having at least one carbon-carbon double bond. Atoms oriented about the double bond are in either the cis (Z) or trans (E) conformation. As used herein, the terms “C₂-C₄alkenyl”, “C₂-C₅alkenyl”, “C₂-C₆alkenyl”, “C₂-C₇alkenyl”, “C₂-C₈alkenyl”, “C₂-C₄alkene”, “C₂-C₅alkene”, “C₂-C₆alkene,” “C₂-C₇alkene”, and “C₂-C₈alkene” refer to an alkyenyl group containing at least 2, and at most 4, 5, 6, 7 or 8 carbon atoms, respectively. Non-limiting examples of alkenyl groups, as used herein, include ethenyl, ethane, propenyl, propene, allyl (2-propenyl), 2-propene, butenyl, butene, pentenyl, pentene, hexenyl, hexene, heptenyl, heptene, octenyl, octene, nonenyl, nonene, decenyl, decene and the like.

The term “alkyl”, as used herein, refers to a saturated branched or straight chain hydrocarbon. As used herein, the terms “C₁-C₃alkyl”, “C₁-C₄alkyl”, “C₁-C₅alkyl”, “C₁-C₆alkyl”, “C₁-C₇alkyl” and “C₁-C₈alkyl” refer to an alkyl group containing at least 1, and at most 3, 4, 5, 6, 7 or 8 carbon atoms, respectively. Non-limiting examples of alkyl groups as used herein include methyl, ethyl, n-propyl, isopropyl, n-butyl, isobutyl, sec-butyl, t-butyl, n-pentyl, isopentyl, hexyl, heptyl, octyl, nonyl, decyl and the like.

The term “alkylene”, as used herein, refers to a saturated branched or straight chain divalent hydrocarbon radical, wherein the radical is derived by the removal of one hydrogen atom from each of two carbon atoms. As used herein, the terms “C₁-C₃alkylene”, “C₁-C₄alkylene”, “C₁-C₅alkylene”, and “C₁-C₆alkylene” refer to an alkylene group containing at least 1, and at most 3, 4, 5 or 6 carbon atoms respectively. Non-limiting examples of alkylene groups as used herein include, methylene, ethylene, n-propylene, isopropylene, n-butylene, isobutylene, sec-butylene, t-butylene, n-pentylene, isopentylene, hexylene and the like.

The term “alkynyl”, as used herein, refers to a partially unsaturated branched or straight chain hydrocarbon radical having at least one carbon-carbon triple bond. As used herein, the terms “C₂-C₄alkynyl”, “C₂-C₅alkynyl”, “C₂-C₆alkynyl”, “C₂-C₇alkynyl”, and “C₂-C₈alkynyl” refer to an alkynyl group containing at least 2, and at most 4, 5, 6, 7 or 8 carbon atoms, respectively. Non-limiting examples of alkynyl groups, as used herein, include ethynyl, propynyl, butynyl, pentynyl, hexynyl, heptynyl, octynyl, nonynyl, decynyl and the like.

The term “alkoxy”, as used herein, refers to the group —ORₐ, where Rₐ is an alkyl group as defined herein. As used herein, the terms “C₁-C₃alkoxy”, “C₁-C₄alkoxy”, “C₁-C₅alkoxy”, “C₁-C₆alkoxy”, “C₁-C₇alkoxy” and “C₁-C₈alkoxy” refer to an alkoxy group wherein the alkyl moiety contains at least 1, and at most 3, 4, 5, 6, 7 or 8, carbon atoms. Non-limiting examples of alkoxy groups, as used herein, include methoxy, ethoxy, n-propoxy, isopropoxy, n-butoxy, t-butoxy, pentoxy, hexoxy, heptoxy, and the like.

The term “aryl”, as used herein, refers to monocyclic, bicyclic, and tricyclic ring systems having a total of six to fourteen ring members, wherein at least one ring in the system is aromatic. Non-limiting examples of aryl groups, as used herein, include phenyl, naphthyl, fluorenyl, indenyl, azulenyl, anthracenyl and the like. An aryl group may contain one or more substituents and thus may be “optionally substituted”. Unless otherwise defined herein, suitable substituents on the unsaturated carbon atom of an aryl group are generally selected from halogen; —R, —OR, —SR, —NO₂, —CN, —N(R)₂, —NRC(O)R, —NRC(S)R, —NRC(O)N(R)₂, —NRC(S)N(R)₂, —NRCO₂R, —NRNRC(O)R, —NRNRC(O)N(R)₂, —NRNRCO₂R, —C(O)C(O)R, —C(O)CH₂C(O)R, —CO₂R, —C(O)R^{o}, —C(S)R, —C(O)N(R)₂, —C(S)N(R)₂, —OC(O)N(R)₂, —OC(O)R, —C(O)N(OR)R, —C(NOR)R, —S(O)₂R, —S(O)₃R, —SO₂N(R)₂, —S(O)R, —NRSO₂N(R)₂, —NRSO₂R, —N(OR)R, —C(═NH)—N(R)₂, —P(O)₂R, —PO(R)₂, —OPO(R)₂, —(CH₂)₀₋₂NHC(O)R, phenyl (Ph) optionally substituted with R, —O(Ph) optionally substituted with R, —(CH₂)₁₋₂(Ph), optionally substituted with R, or —CH═CH(Ph), optionally substituted with R, wherein each independent occurrence of R is selected from hydrogen, optionally substituted C₁-C₆alkyl, optionally substituted C₁-C₆alkoxy, an unsubstituted 5-6 membered heteroaryl, phenyl, —O(Ph), or —CH₂(Ph), or two independent occurrences of R, on the same substituent or different substituents, taken together with the atom(s) to which each R is bound, to form an optionally substituted 3-12 membered saturated, partially unsaturated, or fully unsaturated monocyclic or bicyclic ring having 0-4 heteroatoms independently selected from nitrogen, oxygen, or sulfur.

The term “arylene”, as used means a divalent radical derived from an aryl group.

The term “cyano”, as used herein, refers to a —CN group.

The term “cycloalkyl”, as used herein, refers to a saturated monocyclic, fused bicyclic, fused tricyclic or bridged polycyclic ring assembly. As used herein, the terms “C₃-C₅cycloalkyl”, “C₃-C₆cycloalkyl”, “C₃-C₇cycloalkyl”, “C₃-C₈cycloalkyl, “C₃-C₉cycloalkyl and “C₃-C₁₀cycloalkyl refer to a cycloalkyl group wherein the monocyclic, fused bicyclic or bridged polycyclic ring assembly contain at least 3, and at most 5, 6, 7, 8, 9 or 10, carbon atoms. Non-limiting examples of cycloalkyl groups, as used herein, include cyclopropyl, cyclobutyl, cyclopentyl, cyclohexyl, cycloheptyl, cyclooctyl, cyclononyl, cyclodecyl, cyclopentyl, cyclohexyl, decahydronaphthalenyl, 2,3,4,5,6,7-hexahydro-1H-indenyl and the like.

The term “halogen”, as used herein, refers to fluorine (F), chlorine (Cl), bromine (Br), or iodine (I).

The term “halo”, as used herein, refers to the halogen radicals: fluoro (—F), chloro (—Cl), bromo (—Br), and iodo (—I).

The terms “haloalkyl” or “halo-substituted alkyl”, as used herein, refers to an alkyl group as defined herein, substituted with at least one halo group or combinations thereof. Non-limiting examples of such branched or straight chained haloalkyl groups, as used herein, include methyl, ethyl, propyl, isopropyl, isobutyl and n-butyl substituted with one or more halo groups or combinations thereof, and include, but are not limited to, trifluoromethyl, pentafluoroethyl, and the like.

The term “heteroalkyl”, as used herein, refers to refers to an alkyl group as defined herein wherein one or more carbon atoms are independently replaced by one or more of oxygen, sulfur, nitrogen, or combinations thereof.

The term “heteroaryl”, as used herein, refers to monocyclic, bicyclic, and tricyclic ring systems having a total of five to fourteen ring members, wherein at least one ring in the system is aromatic and at least one ring in the system contains one or more heteroatoms selected from nitrogen, oxygen and sulfur. Non-limiting examples of heteroaryl groups, as used herein, include benzofuranyl, benzofurazanyl, benzoxazolyl, benzopyranyl, benzthiazolyl, benzothienyl, benzazepinyl, benzimidazolyl, benzothiopyranyl, benzo[1,3]dioxole, benzo[b]furyl, benzo[b]thienyl, cinnolinyl, furazanyl, furyl, furopyridinyl, imidazolyl, indolyl, indolizinyl, indolin-2-one, indazolyl, isoindolyl, isoquinolinyl, isoxazolyl, isothiazolyl, 1,8-naphthyridinyl, oxazolyl, oxaindolyl, oxadiazolyl, pyrazolyl, pyrrolyl, phthalazinyl, pteridinyl, purinyl, pyridyl, pyridazinyl, pyrazinyl, pyrimidinyl, quinoxalinyl, quinolinyl, quinazolinyl, 4H-quinolizinyl, thiazolyl, thiadiazolyl, thienyl, triazinyl, triazolyl and tetrazolyl. Unless otherwise defined above and herein, suitable substituents on the unsaturated carbon atom of a heteroaryl group are generally selected from halogen; —R, —OR, —SR, —NO₂, —CN, —N(R)₂, —NRC(O)R, —NRC(S)R, —NRC(O)N(R)₂, —NRC(S)N(R)₂, —NRCO₂R, —NRNRC(O)R, —NRNRC(O)N(R)₂, —NRNRCO₂R, —C(O)C(O)R, —C(O)CH₂C(O)R, —CO₂R, —C(O)R^{o}, —C(S)R, —C(O)N(R)₂, —C(S)N(R)₂, —OC(O)N(R)₂, —OC(O)R, —C(O)N(OR)R, —C(NOR)R, —S(O)₂R, —S(O)₃R, —SO₂N(R)₂, —S(O)R, —NRSO₂N(R)₂, —NRSO₂R, —N(OR)R, —C(═NH)—N(R)₂, —P(O)₂R, —PO(R)₂, —OPO(R)₂, —(CH₂)₀₋₂NHC(O)R, phenyl (Ph) optionally substituted with R, —O(Ph) optionally substituted with R, —(CH₂)₁₋₂(Ph), optionally substituted with R, or —CH═CH(Ph), optionally substituted with R, wherein each independent occurrence of R is selected from hydrogen, optionally substituted C₁-C₆alkyl, optionally substituted C₁-C₆alkoxy, an unsubstituted 5-6 membered heteroaryl, phenyl, —O(Ph), or —CH₂(Ph), or two independent occurrences of R, on the same substituent or different substituents, taken together with the atom(s) to which each R is bound, to form an optionally substituted 3-12 membered saturated, partially unsaturated, or fully unsaturated monocyclic or bicyclic ring having 0-4 heteroatoms independently selected from nitrogen, oxygen, or sulfur.

The term “heterocycloalkyl”, as used herein, refers to a monocyclic ring assembly having a total of three to ten ring members or a or bicyclic ring assembly having up to ten ring members, wherein the ring assembly contains one to three groups selected from —O—, —N═, —NR—, —C(O)—, —S—, —S(O)— or —S(O)₂—, wherein R is hydrogen or an N substituent provided herein, with the proviso that the ring does not contain two adjacent O or S atoms. Non-limiting examples of heterocycloalkyl groups, as used herein, include morpholino, pyrrolidinyl, pyrrolidinyl-2-one, piperazinyl, piperidinyl, piperidinylone, 1,4-dioxa-8-aza-spiro[4.5]dec-8-yl, 2H-pyrrolyl, 2-pyrrolinyl, 3-pyrrolinyl, 1,3-dioxolanyl, 2-imidazolinyl, imidazolidinyl, 2-pyrazolinyl, pyrazolidinyl, 1,4-dioxanyl, 1,4-dithianyl, thiomorpholinyl, azepanyl, hexahydro-1,4-diazepinyl, tetrahydrofuranyl, dihydrofuranyl, tetrahydrothienyl, tetrahydropyranyl, dihydropyranyl, tetrahydrothiopyranyl, thioxanyl, azetidinyl, oxetanyl, thietanyl, oxepanyl, thiepanyl, 1,2,3,6-tetrahydropyridinyl, 2H-pyranyl, 4H-pyranyl, dioxanyl, 1,3-dioxolanyl, dithianyl, dithiolanyl, dihydropyranyl, dihydrothienyl, dihydrofuranyl, imidazolinyl, imidazolidinyl, 3-azabicyclo[3.1.0]hexanyl, and 3-azabicyclo[4.1.0]heptanyl.

The term “heteroatom”, as used herein, refers to one or more of oxygen, sulfur, nitrogen, phosphorus, or silicon.

The term “hydroxyl”, as used herein, refers to the group —OH.

The term “hydroxyalkyl”, as used herein refers to an alkyl group as defined herein substituted with at least one hydroxyl, hydroxyl being as defined herein. Non-limiting examples of branched or straight chained “C₁-C₆ hydroxyalkyl groups as used herein include methyl, ethyl, propyl, isopropyl, isobutyl and n-butyl substituted independently with one or more hydroxyl groups.

The term “isocyanato”, as used herein, refers to a —N═C═O group.

The term “isothiocyanato”, as used herein, refers to a —N═C═S group.

The term “mercaptyl”, as used herein, refers to an (alkyl)S— group.

The term “optionally substituted”, as used herein, means that the referenced group may or may not be substituted with one or more additional group(s) individually and independently selected from alkyl, alkenyl, alkynyl, cycloalkyl, aryl, heteroaryl, heterocycloalkyl, hydroxyl, alkoxy, mercaptyl, cyano, halo, carbonyl, thiocarbonyl, isocyanato, thiocyanato, isothiocyanato, nitro, perhaloalkyl, perfluoroalkyl, and amino, including mono- and di-substituted amino groups, and the protected derivatives thereof. Non-limiting examples of optional substituents include, halo, —CN, —OR, —C(O)R, —OC(O)R, —C(O)OR, OC(O)NHR, —C(O)N(R)₂, —SR—, —S(═O)R, —S(═O)₂R, —NHR, —N(R)₂, —NHC(O)—, NHC(O)O—, —C(O)NH—, S(═O)₂NHR, —S(O)₂N(R)₂, —NHS(═O)₂, —NHS(O)₂R, C₁-C₆ alkyl, C₁-C₆ alkoxy, aryl, heteroaryl, cycloalkyl, heterocycloalkyl, halo-substituted C₁-C₆alkyl, halo-substituted C₁-C₆alkoxy, where each R is independently selected from H, halo, C₁-C₆ alkyl, C₁-C₆ alkoxy, aryl, heteroaryl, cycloalkyl, heterocycloalkyl, halo-substituted C₁-C₆alkyl, halo-substituted C₁-C₆alkoxy.

The term “solvate”, as used herein, refers to a complex of variable stoichiometry formed by a solute (in this invention, a compound of Formula (I), or a salt thereof) and a solvent. Such solvents for the purpose provided herein may not interfere with the biological activity of the solute. Non-limiting examples of suitable solvents include water, acetone, methanol, ethanol and acetic acid. Preferably the solvent used is a pharmaceutically acceptable solvent. Non-liniting examples of suitable pharmaceutically acceptable solvents include water, ethanol and acetic acid.

The term “acceptable” with respect to a formulation, composition or ingredient, as used herein, means having no persistent detrimental effect on the general health of the subject being treated.

The term “administration” or “administering” of the subject compound means providing a compound provided herein and prodrugs thereof to a subject in need of treatment.

The term “bone disease,’ as used herein, refers to a disease or condition of the bone, including, but not limited to, inapproriate bone remodeling, loss or gain, osteopenia, osteomalacia, osteofibrosis, and Paget's disease.

The term “cardiovascular disease,” as used herein refers to diseases affecting the heart or blood vessels or both, including but not limited to: arrhythmia; atherosclerosis and its sequelae; angina; myocardial ischemia; myocardial infarction; cardiac or vascular aneurysm; vasculitis, stroke; peripheral obstructive arteriopathy of a limb, an organ, or a tissue; reperfusion injury following ischemia of the brain, heart or other organ or tissue; endotoxic, surgical, or traumatic shock; hypertension, valvular heart disease, heart failure, abnormal blood pressure; shock; vasoconstriction (including that associated with migraines); vascular abnormality, inflammation, insufficiency limited to a single organ or tissue.

The term “cancer,” as used herein refers to an abnormal growth of cells which tend to proliferate in an uncontrolled way and, in some cases, to metastasize (spread). The types of cancer include, but is not limited to, solid tumors (such as those of the bladder, bowel, brain, breast, endometrium, heart, kidney, lung, lymphatic tissue (lymphoma), ovary, pancreas or other endocrine organ (thyroid), prostate, skin (melanoma) or hematological tumors (such as the leukemias).

The term “carrier,” as used herein, refers to chemical compounds or agents that facilitate the incorporation of a compound provided herein into cells or tissues.

The terms “co-administration” or “combined administration” or the like as used herein are meant to encompass administration of the selected therapeutic agents to a single patient, and are intended to include treatment regimens in which the agents are not necessarily administered by the same route of administration or at the same time.

The term “dermatological disorder,” as used herein refers to a skin disorder. Such dermatological disorders include, but are not limited to, proliferative or inflammatory disorders of the skin such as, atopic dermatitis, bullous disorders, collagenoses, contact dermatitis eczema, Kawasaki Disease, rosacea, Sjogren-Larsso Syndrome, and urticaria.

The term “diluent” as used herein, refers to chemical compounds that are used to dilute a compound provided herein prior to delivery. Diluents can also be used to stabilize compounds provided herein.

The terms “effective amount” or “therapeutically effective amount,” as used herein, refer to a sufficient amount of a compound provided herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “fibrosis” or “fibrosing disorder,” as used herein, refers to conditions that follow acute or chronic inflammation and are associated with the abnormal accumulation of cells and/or collagen and include but are not limited to fibrosis of individual organs or tissues such as the heart, kidney, joints, lung, or skin, and includes such disorders as idiopathic pulmonary fibrosis and cryptogenic fibrosing alveolitis.

The term “iatrogenic”, as used herein, means a condition, disorder, or disease created or worsened by medical or surgical therapy.

The term “inflammatory disorders”, as used herein, refers to those diseases or conditions that are characterized by one or more of the signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and loss of function (functio laesa, which may be partial or complete, temporary or permanent). Inflammation takes many forms and includes, but is not limited to, inflammation that is one or more of the following: acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders further include, without being limited to those affecting the blood vessels (polyarteritis, temporarl arteritis); joints (arthritis: crystalline, osteo-, psoriatic, reactive, rheumatoid, Reiter's); gastrointestinal tract (Disease,); skin (dermatitis); or multiple organs and tissues (systemic lupus erythematosus).

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The terms “neurogenerative disease” or “nervous system disorder,” as used herein, refers to conditions that alter the structure or function of the brain, spinal cord or peripheral nervous system, including but not limited to Alzheimer's Disease, cerebral edema, cerebral ischemia, multiple sclerosis, neuropathies, Parkinson's Disease, those found after blunt or surgical trauma (including post-surgical cognitive dysfunction and spinal cord or brain stem injury), as well as the neurological aspects of disorders such as degenerative disk disease and sciatica. The acronym “CNS” refers to disorders of the central nervous system (brain and spinal cord).

The terms “ocular disease” or “ophthalmic disease,” as used herein, refer to diseases which affect the eye or eyes and potentially the surrounding tissues as well. Ocular or ophthalmic diseases include, but are not limited to, conjunctivitis, retinitis, scleritis, uveitis, allergic conjuctivitis, vernal conjunctivitis, pappillary conjunctivitis.

The term “pharmaceutically acceptable,” as used herein, refers a material, such as a carrier or diluent, which does not abrogate the biological activity or properties of the compounds provided herein. Such materials are administered to an individual without causing undesirable biological effects or interacting in a deleterious manner with any of the components of the composition in which it is contained.

The term “pharmaceutically acceptable salt”, as used herein, refers to a formulation of a compound that does not cause significant irritation to an organism to which it is administered and does not abrogate the biological activity and properties of the compounds provided herein.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a coagent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

The term “pharmaceutical composition”, as used herein, refers to a mixture of a compound provided herein with other chemical components, such as carriers, stabilizers, diluents, dispersing agents, suspending agents, thickening agents, and/or excipients.

The term “prodrug”, as used herein, refers to an agent that is converted into the parent drug in vivo. Prodrugs are often useful because, in some situations, they may be easier to administer than the parent drug. Prodrugs are bioavailable by oral administration whereas the parent is not. Prodrugs improve solubility in pharmaceutical compositions over the parent drug. A non-limiting example of a prodrug of the compounds provided herein is a compound provided herein administered as an ester which is then metabolically hydrolyzed to a carboxylic acid, the active entity, once inside the cell. A further example of a prodrug is a short peptide bonded to an acid group where the peptide is metabolized to reveal the active moiety.

The term “respiratory disease,” as used herein, refers to diseases affecting the organs that are involved in breathing, such as the nose, throat, larynx, trachea, bronchi, and lungs. Respiratory diseases include, but are not limited to, asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia.

The term “subject” or “patient”, as used herein, encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish and the like.

The term “Syk inhibitor”, as used herein, refers to a compound which inhibits the Syk receptor.

The term “Syk mediated disease” or a “disorder or disease or condition mediated by inappropriate Syk activity”, as used herein, refers to any disease state mediated or modulated by Syk kinase mechanisms. Such disease states include, but are not limited to, an inflammatory disease, an allergic disease, a cell-proliferative disease, an autoimmune disease and cytopenia, such as, by way of example only, allergic asthma, allergic rhinitis, rheumatoid arthritis, multiple sclerosis, lupus, systemic lupus erythematosus, lymphoma, B cell lymphoma, T cell lymphoma, myelodysplasic syndrome, anemia, leucopenia, neutropenia, thrombocytopenia, granuloctopenia, pancytoia or idiopathic thrombocytopenic purpura.

The term “therapeutically effective amount”, as used herein, refers to any amount of a compound which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The terms “treat”, “treating” or “treatment,” as used herein, refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

The compound names provided herein were obtained using ChemDraw Ultra 10.0 (CambridgeSoft®) or JChem version 5.2.2 (ChemAxon).

Other objects, features and advantages of the methods and compositions provided herein will become apparent from the following detailed description. It should be understood, however, that the detailed description and the specific examples, while indicating specific embodiments, are given by way of illustration only.

### DESCRIPTION OF THE PREFERRED EMBODIMENTS

Provided herein are compounds and pharmaceutical compositions thereof, which are Syk kinase inhibitors. Also provided herein are compounds, pharmaceutical compositions and methods for the treatment and/or prevention of Syk kinase mediated diseases or conditions/disorders, including diseases or conditions/disorders associated with abnormal or deregulated Syk kinase activity.

The Syk kinase inhibitors provided herein are compounds having a structure of Formula (I), and pharmaceutically acceptable salts, pharmaceutically acceptable solvates (e.g. hydrates), the N-oxide derivatives, individual stereoisomers and mixture of stereoisomers thereof:

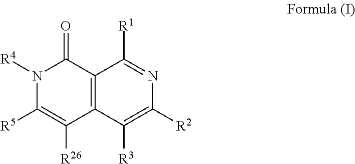

wherein:
R¹ is —NR⁶R⁷ or heterocycloalkyl optionally substituted with 1 to 3 substituents independently selected from halogen, hydroxyl, hydroxyl-C₁-C₆alkyl, —(CR⁹R⁹)ₙOR⁹, R¹⁰, ═N—OH, —(CR⁹R⁹)ₙSR⁹, —(CR⁹R⁹)ₙOS(O)₂N(R⁹)₂, —(CR⁹R⁹)ₙOS(O)₂N(R⁹)₂, —(CR⁹R⁹)ₙN₃, —(CR⁹R⁹)ₙNR⁹R⁹, —(CR⁹R⁹)ₙC(O)NR⁹R⁹, —(CR⁹R⁹)ₙC(O)OR⁹ and —(CR⁹R⁹)ₙC(O)R⁹;
R² is selected from —NR⁸R¹⁰, R¹⁵, —C(O)R¹², —(CR¹²R¹²)ₙR¹⁴, —CR¹²═NOR¹², C₁-C₆alkyl, C₂-C₆alkene, aryl, heteroaryl and heterocycloalkyl, wherein the aryl, C₁-C₆alkyl, C₂-C₆alkene, heteroaryl, and heterocycloalkyl of R² is optionally substituted with 1 to 3 substituents independently selected from —OR¹², —C(O)¹⁰, —C(O)OR¹²—C(O)R¹⁰, —N(R¹²)₂, —(CR¹²R¹²)ₙR¹⁴, —C₁-C₆alkyl and hydroxyl-C₁-C₆alkyl;
R⁴ is H, C₁-C₆alkyl, deuterated C₁-C₆alkyl, —CD₃, C₁-C₆haloalkyl, C₂-C₆alkene, hydroxyl-C₁-C₆alkyl, —R¹⁵, —(CR²⁷R²⁷)₁₋₆R¹⁴, —(CR²⁷R²⁷)(CR²⁷R²⁵)R¹¹, —(CR²⁷R²⁷)(CR²⁷R²⁵)R²⁵, —C(R²⁷R²⁵R²⁵) or —(CR²⁷R²⁷)ₙR¹¹, —(CR³R³)ₙR¹⁴ or —(CR³R³)ₙR¹¹;
each R³ and each R⁵ are independently selected from H, halogen and C₁-C₆alkyl;
R⁶ is H, aryl, heteroaryl, heterocycloalkyl, C₁-C₆alkyl, C₃-C₈cycloalkyl, R¹⁵, —S(O)₂R¹³, —(CR¹²R¹²)ₙR¹⁴ or —(CR¹²R¹²)ₙR¹⁰, wherein the aryl, heteroaryl, C₁-C₆alkyl, heterocycloalkyl and C₃-C₈cycloalkyl of R⁶ are optionally substituted with 1 to 3 substituents independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —C(O)R¹², —C(O)R¹³, —C(O)R¹⁵, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)ₙR¹⁴, —O(CR¹²R¹²)ₙR¹⁰, —(CR¹²R¹²)ₙC(O)N(R¹²)₂, —C(O)N(R¹²)(CR¹²R¹²)ₙR¹⁴, —C(R¹²R¹²R¹⁴), —(CR¹²R¹²)ₙR¹¹, —C(O)(CR¹²R¹²)ₙR¹⁴, —C(O)C(R¹²R¹²R¹⁴), —NR¹²R¹², —S(O)₂NR¹²(CR¹²R¹²)₁₋₆R¹⁴, —S(O)₂NR¹²R¹², —S(O)₂R¹²—C(O)C(R¹²R¹⁴)(C(R¹²R¹²))ₙR¹⁴, —(CR¹²R¹²)ₙC(O)OR¹², —C(O)N(R¹²)(CR¹²R¹²)ₙR¹¹, —(CR¹²R¹²)ₙC(O)R¹⁴, —(CR¹²R¹²)ₙC(R¹²R¹⁴)(C(R¹²R¹²))ₙR¹⁴ and —(CR¹²R¹²)ₙC(O)NR¹²(CR¹²R¹²)ₙR¹⁴;
R⁷ is H or C₁-C₆ alkyl;
R⁸ is H or C₁-C₆ alkyl;
each R⁹ is independently selected from H and C₁-C₆alkyl;
R¹⁰ is aryl, heteroaryl, a heteroaryl N-oxide, heterocycloalkyl, C₃-C₈cycloalkyl or —(CR¹²R¹²)ₙR¹¹, wherein the aryl, heteroaryl, heterocycloalkyl and C₃-C₈cycloalkyl of R¹⁰ are optionally substituted with 1 to 3 substituents independently selected from halogen, hydroxyl, —NO₂, —CN, —C₁-C₆alkyl, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, —OR¹², —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴, —O(CR¹²R¹²)₁₋₆R¹⁴, —O(CR¹²R¹²)ₙR¹¹, —(CR³R³)₁₋₆R¹⁴, —(CR¹²R¹²)ₙC(O)NR¹²(CR¹²R¹²)ₙR¹⁴, C(O)NR²⁷(CR¹²R¹²)ₙR¹¹, —(CR¹²R¹²)ₙC(O)NR¹²OR¹², —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₆R¹⁴, —(CR¹²R¹²)ₙC(O)R¹⁴, —C(O)C(R¹²R¹⁴)(C(R¹²R¹²))ₙR¹⁴, R¹⁵, R¹¹, —C(O)(CR¹²R¹²)ₙR¹⁴, and —C(O)C(R¹²R¹²R¹⁴);
R¹¹ is aryl, heteroaryl, C₃-C₈cycloalkyl, or heterocycloalkyl, wherein the aryl, heteroaryl, C₃-C₈cycloalkyl and heterocycloalkyl of R¹¹ are optionally substituted with 1 to 3 substituents independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴;
each R¹² is independently selected from H, C₁-C₆alkyl, hydroxyl-C₁-C₆alkyl and C₃-C₈cycloalkyl, or each R¹² is independently a C₁-C₆alkyl that together with N they are attached form a heterocycloalkyl, or each R^{u} is independently a C₁-C₆alkyl that together with C they are attached form a C₃-C₈cycloalkyl;
R¹³ is H, C₁-C₆alkyl, halo-substituted C₁-C₆alkyl or heterocycloalkyl;
R¹⁴ is H, halogen, hydroxyl, hydroxyl-C₁-C₆alkyl, —OR¹³, —OR¹², —O(CR¹²R¹²)ₙOR¹³, —C(O)R¹³, —N(R¹²)₂, —NR¹²OR¹³, —CN, —C(O)N(R¹²)₂, —S(O)₂R¹³, R¹³, —(CR¹²R¹²)ₙOR¹³, —C(O)R¹⁰, —OC(O)R¹³, —C(O)OR¹³, —S(O)₂N(R¹²)₂, —N(R¹²R¹⁰), —N(R¹²R¹⁰, —(CR¹²R¹²)ₙN(R¹²)₂, —NR¹²C(O)(R¹²), —(CR¹²R¹²)ₙR¹³, —N(R¹²)C(O)(CR¹²R¹²)ₙOR¹³, N(R¹²)(CR¹²R¹²)ₙOR¹³, N(R¹²)(CR¹²R¹²)ₙR¹⁰, —C(O)N(R¹²)₂, —N(R¹²)C(O)R¹³, —N(R¹²)C(O)OR¹³, —(CR¹²R¹²)ₙR¹⁰, and R¹⁵;
R¹⁵ is

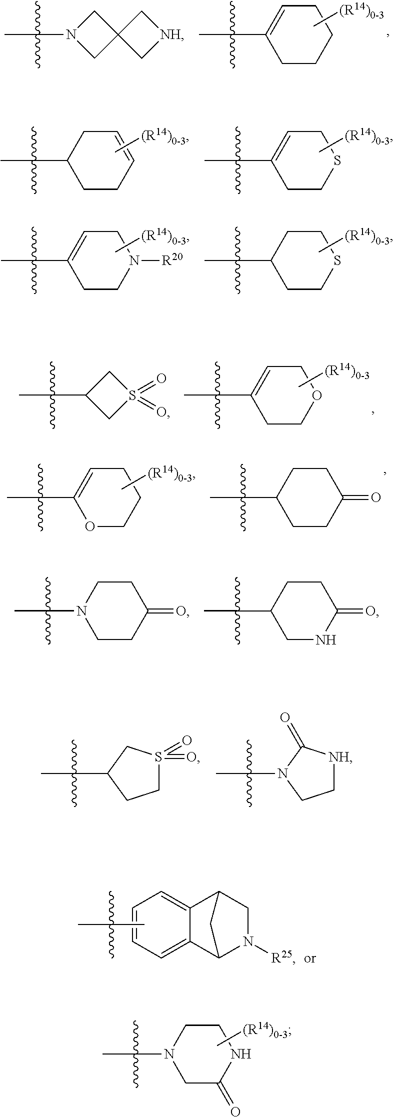

R²⁰ is H, —C₁-C₆alkyl, hydroxyl-C₁-C₆alkyl, —(CR¹²R¹²)₁₋₆R¹⁴ or (CR¹²R¹²)ₙC(O)R¹³;
each R²⁵ is independently selected from H, hydroxyl, —C₁-C₆alkyl, —C₁-C₆haloalkyl, hydroxyl-C₁-C₆alkyl and —(CR¹²R¹²)ₙR¹⁴;
R²⁶ is H, halogen or C₁-C₆ alkyl;
each R²⁷ is independently selected from H or C₁-C₆alkyl, and
each n is independently 0, 1, 2, 3, 4, 5 or 6.

In certain embodiments of such compounds of Formula (I):
R¹ is —NR⁶R⁷, a 4-8 membered heterocycloalkyl containing 1 to 2 heteroatoms independently selected from N, O and S or a 4-8 membered heterocycloalkyl containing 1 to 2 heteroatoms independently selected from N, O and S which is substituted with 1 to 3 substituents independently selected from hydroxyl and hydroxyl-C₁-C₆alkyl;
R² is selected from —NR⁸R¹⁰, R¹⁵, —C(O)R¹², —(CR¹²R¹²)ₙR¹⁴, CR¹²═NOR¹², C₁-C₆alkyl, C₂-C₆alkene, phenyl, C₁₀aryl, C₁₄aryl, a 5, 6, 9, 10 or 14 membered heteroaryl containing 1 to 3 heteroatoms independently selected from N, O and S, and a 4-8 membered heterocycloalkyl containing 1 to 2 heteroatoms independently selected from N, O and S,
or R² is selected from C₁-C₆alkyl, C₂-C₆alkene, phenyl, C₁₀aryl, C₁₄aryl, a 5, 6, 9, 10 or 14 membered heteroaryl containing 1 to 3 heteroatoms independently selected from N, O and S, and a 4-8 membered heterocycloalkyl containing 1 to 2 heteroatoms independently selected from N, O and S, each of which is substituted with 1 to 3 substituents independently selected from —OR¹², —OR¹⁰, —C(O)OR¹², —C(O)R¹⁰, —N(R¹²)₂, —(CR¹²R¹²)ₙR¹⁴; —C₁-C₆alkyl and hydroxyl-C₁-C₆alkyl;
R⁴ is H, C₁-C₆alkyl, deuterated C₁-C₆alkyl, —CD₃, C₁-C₆haloalkyl, C₂-C₆alkene, hydroxyl-C₁-C₆alkyl, R¹⁵, —(CR²⁷R²⁷)₁₋₆R¹⁴, —(CR²⁷R²⁷)(CR²⁷R²⁵)R¹¹, —C(R²⁷R²⁷)(CR²⁷R²⁵)R²⁵, —C(R²⁷R²⁵R²⁵) or —(CR²⁷R²⁷)ₙR¹¹;
each R³ and each R⁵ are independently selected from H, halogen and C₁-C₆alkyl;
R⁶ is H, phenyl, C₁₀aryl, C₁₄aryl, C₁-C₆alkyl, C₃-C₈cycloalkyl, R¹⁵, —S(O)₂R¹³, —(CR¹²R¹²)₁₋₆R¹⁰, a 5, 6, 9, 10 or 14 membered heteroaryl containing 1 to 3 heteroatoms independently selected from N, O and S, or a 4-8 membered heterocycloalkyl containing 1 to 2 heteroatoms independently selected from N, O and S,
or R⁶ is phenyl, C₁₀aryl, C₁₄aryl, C₁-C₆alkyl, C₃-C₈cycloalkyl, a 5, 6, 9, 10 or 14 membered heteroaryl containing 1 to 3 heteroatoms independently selected from N, O and S, or a 4-8 membered heterocycloalkyl containing 1 to 2 heteroatoms independently selected from N, O and S, each of which is substituted with 1 to 3 substituents independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —C(O)R¹², —C(O)R¹³, —C(O)R¹⁵, —(CR¹²R¹²)ₙR¹⁴; (CR¹²R¹²)ₙR¹⁰, —(CR¹²R¹²)ₙC(O)R¹³, —(CR¹²R¹²)ₙR¹⁵; —(CR¹²R¹²)ₙC(O)R¹⁰, —O(CR¹²R¹²)₁₋₆R¹⁴; —O(CR¹²R¹²)ₙR¹⁰, —(CR¹²R¹²)ₙC(O)N(R¹²)₂, —C(O)N(R¹²)(CR¹²R¹²)₁₋₆R¹⁴, —C(R¹²R¹²R¹⁴), —(CR¹²R¹²)ₙR¹¹, —C(O)(CR¹²R¹²)₁₋₆R¹⁴, —C(O)C(R¹²R¹²R¹⁴), —NR¹²R¹², —S(O)₂NR¹²(CR¹²R¹²)₁₋₆R¹⁴, —S(O)₂NR¹²R¹², —S(O)₂R¹², C(O)C(R¹²R¹⁴)(C(R¹²R¹²))ₙR¹⁴, —(CR¹²R¹²)ₙC(O)OR¹², —C(O)N(R¹²(CR¹²R¹²)ₙR¹¹, —(CR¹²R¹²)ₙC(O)R¹⁴, —(CR¹²R¹²)ₙC(R¹²R¹⁴)(C(R¹²R¹²))ₙR¹² and —(CR¹²R¹²)ₙC(O)NR¹²(CR¹²R¹²)₁₋₆R¹⁴;
R⁷ is H or C₁-C₆ alkyl;
R⁸ is H or C₁-C₆ alkyl;
R¹⁰ is phenyl, C₁₀aryl, C₁₄aryl, a 5, 6, 9, 10 or 14 membered heteroaryl containing 1 to 3 heteroatoms independently selected from N, O and S, an N-oxide of a 5-6 membered heteroaryl containing 1-3 N heteroatoms, a 4-8 membered heterocycloalkyl containing 1 to 2 heteroatoms independently selected from N, O and S, C₃-C₈cycloalkyl or —(CR¹²R¹²)ₙR¹¹,
or R¹⁰ is phenyl, C₁₀aryl, C₁₄aryl, a 5, 6, 9, 10 or 14 membered heteroaryl containing 1 to 3 heteroatoms independently selected from N, O and S, an N-oxide of a 5-6 membered heteroaryl containing 1-3 N heteroatoms, a 4-8 membered heterocycloalkyl containing 1 to 2 heteroatoms independently selected from N, O and S, or C₃-C₈cycloalkyl, each of which is substituted with 1 to 3 substituents independently selected from halogen, hydroxyl, —NO₂, —CN, —Cₜ—C₆alkyl, —C₂-C₆alkene, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, R¹⁵, R¹¹, —OR¹², —OR¹¹, —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴; —O(CR¹²R¹²)₁₋₆R¹⁴, —O(CR¹²R¹²)ₙR¹¹, —(CR³R³)₁₋₆R¹⁴, —(CR¹²R¹²)ₙC(O)NR¹²(CR¹²R¹²)₁₋₆R¹⁴, —C(O)NR²⁷(CR¹²R¹²)ₙR¹¹, —(CR¹²R¹²)ₙC(O)NR¹²OR¹², —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₆R¹⁴, —C(O)(CR¹²R¹²)₁₋₆R¹⁴, and —C(O)C(R¹²R¹²R¹⁴);
R¹¹ is phenyl, C₁₀aryl, C₁₄aryl, a 5, 6, 9, 10 or 14 membered heteroaryl containing 1 to 4 heteroatoms independently selected from N, O and S, C₃-C₈cycloalkyl, or a 4-8 membered heterocycloalkyl containing 1 to 2 heteroatoms independently selected from N, O and S,
or R¹¹ is phenyl, C₁₀aryl, C₁₄aryl, a 5, 6, 9, 10 or 14 membered heteroaryl containing 1 to 4 heteroatoms independently selected from N, O and S, C₃-C₈cycloalkyl, or a 4-8 membered heterocycloalkyl containing 1 to 2 heteroatoms independently selected from N, O and S, each of which is substituted with 1 to 3 substituents independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴;
each R¹² is independently selected from H, C₁-C₆alkyl, hydroxyl-C₁-C₆alkyl and C₃-C₈cycloalkyl, or each R¹² is independently a C₁-C₆alkyl that together with N they are attached form a heterocycloalkyl;
R¹³ is H, C₁-C₆alkyl, halo-substituted C₁-C₆alkyl or a 4-8 membered heterocycloalkyl containing 1 to 2 heteroatoms independently selected from N, O and S;
R¹⁴ is H, halogen, hydroxyl, hydroxyl-C₁-C₆alkyl, R¹³, —OR¹³, —OR¹², —O(CR¹²R¹²)ₙOR¹³, —C(O)R¹³, —N(R¹²)₂, —NR¹²OR¹³, —CN, —C(O)N(R¹²)₂, —S(O)₂R¹³—C(O)R¹⁰, —OC(O)R¹³, —C(O)OR¹³, —S(O)₂N(R¹²)₂, —N(R¹²R¹⁰), —N(R¹²R¹¹), —(CR¹²R¹²)ₙN(R¹²)₂, —NR¹²C(O)(R¹²), —(CR¹²R¹²)ₙR¹³, —N(R¹²)C(O)(CR¹²R¹²)ₙOR¹³, —N(R¹²)(CR¹²R¹²)ₙOR¹³, —N(R¹²)(CR¹²R¹²)ₙR¹⁰, —C(O)N(R¹²)₂, —N(R¹²)C(O)R¹³, —N(R¹²)C(O)OR¹³, —(CR¹²R¹²)ₙR¹⁰, and R¹⁵;
R¹⁵ is

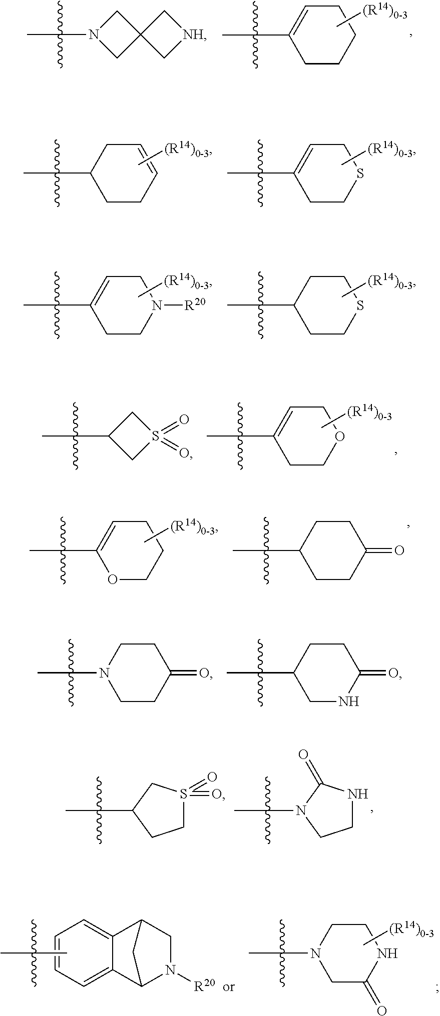

R²⁰ is H, —C₁-C₆alkyl, hydroxyl-C₁-C₆alkyl, —(CR¹²R¹²)₁₋₆R¹⁴ or —(CR¹²R¹²)ₙC(O)R¹³;
each R²⁵ is independently selected from H, hydroxyl, —C₁-C₆alkyl, —C₁-C₆haloalkyl, hydroxyl-C₁-C₆alkyl and —(CR¹²R¹²)ₙR¹⁴;
R²⁶ is H, halogen or C₁-C₆ alkyl;
each R²⁷ is independently selected from H or C₁-C₆alkyl, and
each n is independently 0, 1, 2, 3, 4, 5 or 6.

In certain embodiments of the compounds of Formula (I), R¹ is —NR⁶R⁷, and certain Syk kinase inhibitors provided herein are compounds having a structure of Formula (II), and pharmaceutically acceptable salts, pharmaceutically acceptable solvates (e.g. hydrates), the N-oxide derivatives, individual stereoisomers and mixture of stereoisomers thereof:

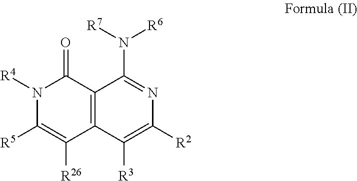

In certain embodiments of such compounds of Formulas (I) and Formula (II), R⁷ is H. In certain embodiments of such compounds of Formulas (I) and Formula (II), R⁸ is H. In certain embodiments of such compounds of Formulas (I) and Formula (II), R⁷ and R⁸ are H.

In certain embodiments of the Syk kinase inhibitors provided herein having the structure of Formula (I) and Formula (II), R⁶ is aryl, heteroaryl, heterocycloalkyl, C₁-C₆alkyl, C₃-C₈cycloalkyl, R¹⁵, —(CR¹²R¹²)ₙR¹⁴ or —(CR¹²R¹²)ₙR¹⁰, wherein the aryl, heteroaryl, C₁-C₆alkyl, heterocycloalkyl and C₃-C₈cycloalkyl of R⁶ are optionally substituted with 1 to 3 substituents independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —C(O)R¹², —C(O)R¹³, —C(O)R¹⁵, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)₁₋₆R¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —C(R¹²R¹²R¹⁴), —(CR¹²R¹²)ₙR¹¹, —C(O)(CR¹²R¹²)₁₋₆R¹⁴, —C(O)C(R¹²R¹²R¹⁴), —NR¹²R¹², —S(O)₂NR¹²(CR¹²R¹²)₁₋₆R¹⁴, —S(O)₂NR¹²R¹², —S(O)₂R¹², C(O)C(R¹²R¹⁴)(C(R¹²R¹²))ₙR¹⁴, —(CR¹²R¹²)ₙC(O)OR¹², —C(O)N(R¹²)(CR¹²R¹²)ₙR¹¹, —(CR¹²R¹²)ₙC(O)R¹⁴, —(CR¹²R¹²)ₙC(R¹²R¹⁴)(C(R¹²R¹²))ₙR¹⁴ and —(CR¹²R¹²)ₙC(O)NR¹²(CR¹²R¹²)₁₋₆R¹⁴, and each n is independently 0, 1, 2, 3 or 4.

In certain embodiments of the Syk kinase inhibitors provided herein having the structure of Formula (I) and Formula (II), R⁶ is H, phenyl, C₁-C₆alkyl, C₃-C₈cycloalkyl, R¹⁵, —S(O)₂R¹³, —(CR¹²R¹²)₁₋₆R¹⁰, a 5, 6, 9, 10 or 14 membered heteroaryl containing 1 to 3 heteroatoms independently selected from N, O and S, or a 4-8 membered heterocycloalkyl containing 1 to 2 heteroatoms independently selected from N, O and S, and each n is independently 0, 1, 2, 3 or 4, while in other embodiments of the aforementioned compounds of Formula (I), R⁶ is phenyl, C₁-C₆alkyl, C₃-C₈cycloalkyl, a 5, 6, 9, 10 or 14 membered heteroaryl containing 1 to 3 heteroatoms independently selected from N, O and S, or a 4-8 membered heterocycloalkyl containing 1 to 2 heteroatoms independently selected from N, O and S, each of which is substituted with 1 to 3 substituents independently selected from halogen, hydroxyl, —C₁-C₆alkyl, —Cₜ—C₆haloalkyl, deuterium, hydroxyl-C₁-C₆alkyl, —OR¹², R¹⁰, R¹⁵, —C(O)R¹⁰, —C(O)R¹¹, —C(O)R¹², —C(O)R¹³, —C(O)R¹⁵, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)₁₋₆R¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —C(R¹²R¹²R¹⁴), —(CR¹²R¹²)ₙR¹¹, —C(O)(CR¹²R¹²)₁₋₆R¹⁴, —C(O)C(R¹²R¹²R¹⁴), —NR¹²R¹², —S(O)₂NR¹²(CR¹²R¹²)₁₋₆R¹⁴, —S(O)₂NR¹²R¹², —S(O)₂R¹², —(CR¹²R¹²)ₙC(O)OR¹², C(O)C(R¹²R¹⁴)(C(R¹²R¹²))ₙR¹⁴, —C(O)N(R¹²)(CR¹²R¹²)ₙR¹¹, —(CR¹²R¹²)ₙC(O)R¹⁴, —(CR¹²R¹²)ₙC(R¹²R¹⁴)(C(R¹²R¹²))ₙR¹⁴ and —(CR¹²R¹²)ₙC(O)NR¹²(CR¹²R¹²)₁₋₆R¹⁴, and each n is independently 0, 1, 2, 3 or 4.

In other embodiments of the Syk kinase inhibitors provided herein having the structure of Formula (I) and Formula (II), R⁶ is aryl, heteroaryl, heterocycloalkyl, C₃-C₈cycloalkyl, each of which is optionally substituted with 1 to 3 substituents independently selected hydroxyl, —C₁-C₆alkyl, hydroxyl-C₁-C₆alkyl, —OR¹², R¹⁰, R¹⁵, —C(O)R¹⁰, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)ₙR¹⁴, —(CR¹²R¹²)ₙC(O)N(R¹²)₂, —C(O)N(R¹²)(CR¹²R¹²)ₙR¹⁴ and —C(O)N(R¹²)(CR¹²R¹²)ₙR¹¹, and each n is independently 0, 1, 2, 3 or 4.

In certain embodiments of the Syk kinase inhibitors provided herein having the structure of Formula (I) and Formula (II), R⁶ is H, phenyl, C₁-C₆alkyl, C₃-C₈cycloalkyl, R¹⁵, —S(O)₂R¹³, —(CR¹²R¹²)₁₋₆R¹⁰, a 5, 6 or 9 membered heteroaryl containing 1 to 3 heteroatoms independently selected from N, O and S, or a 4-8 membered heterocycloalkyl containing 1 to 2 heteroatoms independently selected from N, O and S, and each n is independently 0, 1, 2, 3 or 4, while in other embodiments R⁶ is phenyl, C₁-C₆alkyl, C₃-C₈cycloalkyl, a 5, 6 or 9 membered heteroaryl containing 1 to 3 heteroatoms independently selected from N, O and S, or a 4-8 membered heterocycloalkyl containing 1 to 2 heteroatoms independently selected from N, O and S, each of which is optionally substituted with 1 to 3 substituents independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —(CR¹²R¹²)₁₋₆R¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)ₙR¹⁴, —O(CR¹²R¹²)ₙR¹⁰, (CR¹²R¹²)ₙC(O)N(R¹²)₂, —C(O)N(R¹²)(CR¹²R¹²)₁₋₆R¹⁴, —(CR¹²R¹²)ₙR¹¹, —NR¹²R¹², —S(O)₂NR¹²(CR¹²R¹²)₁₋₆R¹⁴, —S(O)₂NR¹²R¹², —S(O)₂R¹², —(CR¹²R¹²)ₙC(O)NR¹²(CR¹²R¹²)₁₋₆R¹⁴, and —C(O)N(R¹²)(CR¹²R¹²)ₙR¹¹, and each n is independently 0, 1, 2, 3 or 4.

In certain embodiments of the Syk kinase inhibitors provided herein having the structure of Formula (I) and Formula (II), R⁶ is

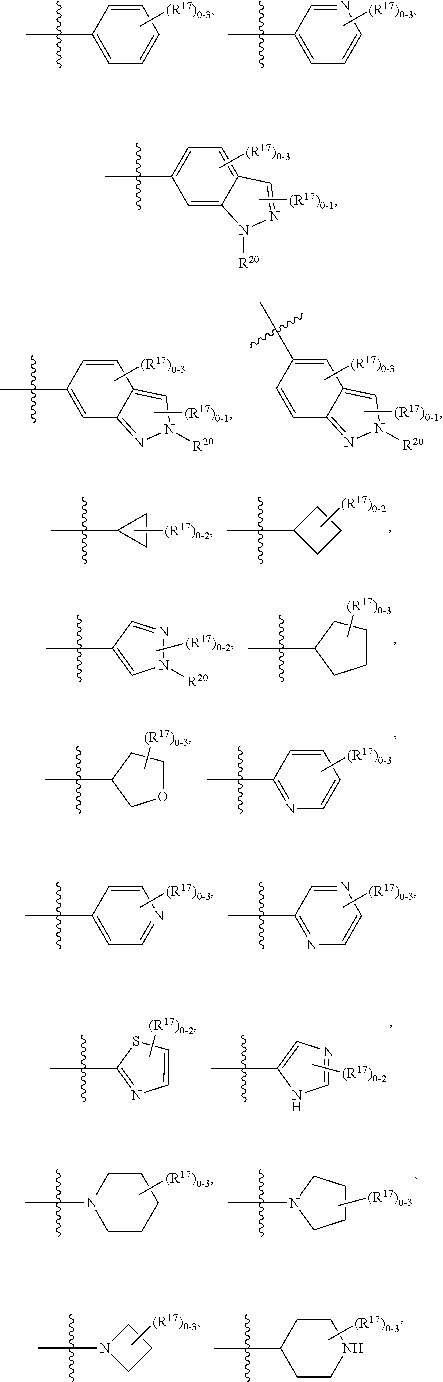

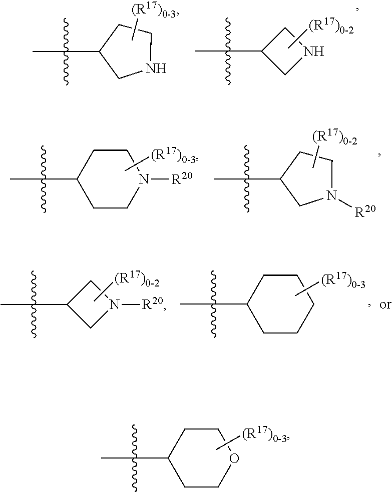

wherein each R¹⁷ is independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)₁₋₆R¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —(CR¹²R¹²)ₙR¹¹, —NR¹²R¹², —S(O)₂NR¹²(CR¹²R¹²)₁₋₆R¹⁴, —S(O)₂NR¹²R¹², —S(O)₂R¹², —(CR¹²R¹²)ₙC(O)NR¹²(CR¹²R¹²)₁₋₆R¹⁴, and —C(O)N(R¹²)(CR¹²R¹²)ₙR¹¹; R²⁰ is H, hydroxyl, —C₁-C₆alkyl, hydroxyl-C₁-C₆alkyl, —OR¹², R¹⁰, R¹⁵, —C(O)R¹⁰, —(CR¹²R¹²)ₙR¹⁴, —(CR¹²R¹²)ₙR¹⁰, —(CR¹²R¹²)ₙC(O)R¹³, —(CR¹²R¹²)ₙC(O)N(R¹²)₂, —C(O)N(R¹²)(CR¹²R¹²)ₙR¹⁴, —C(O)N(R¹²)(CR¹²R¹²)ₙR¹¹ or

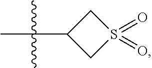

and each n is independently 0, 1, 2, 3 or 4.

In certain embodiments of such R⁶ groups, R²⁰ is H, —C₁-C₆alkyl, hydroxyl-C₁-C₆alkyl or —(CR¹²R¹²)ₙR¹⁰.

In certain embodiments of the Syk kinase inhibitors provided herein having the structure of Formula (I) and Formula (II), R⁶ is —(CR¹²R¹²)ₙR¹⁴. In certain embodiments of such Syk kinase inhibitors provided herein having the structure of Formula (I) and Formula (II), R¹⁴ is selected from halogen, hydroxyl, hydroxyl-C₁-C₆alkyl, —OR¹³, —O(CR¹²R¹²)ₙOR¹³, C(O)R¹³, —N(R¹²)₂, —NR¹²OR¹³, —CN, —C(O)N(R¹²)₂, —S(O)₂R¹³ and R¹³.

In certain embodiments of the aforementioned compounds of Formula (I), R⁶ is C₁-C₆alkyl or C₁-C₆alkyl substituted with 1 to 3 substituents independently selected from halogen, hydroxyl, C₁-C₆alkyl, C₁-C₆haloalkyl, hydroxyl-C₁-C₆alkyl, —OR¹², —O(CR¹²R¹²)ₙOR¹³, —C(O)R¹³, —N(R¹²)₂, —NR¹²OR¹³, —CN, —C(O)N(R¹²)₂, —S(O)₂R¹³ and R¹³.

In certain embodiments of the Syk kinase inhibitors provided herein having the structure of Formula (I), R¹ is a heterocycloalkyl optionally substituted with 1 to 3 substituents independently selected from halogen, hydroxyl, hydroxyl-C₁-C₆alkyl, —(CR⁹R⁹)ₙOR⁹, R¹⁰, ═N—OH, —(CR⁹R⁹)ₙSR⁹, —(CR⁹R⁹)ₙOS(O)₂N(R⁹)₂, —(CR⁹R⁹)ₙOS(O)₂N(R⁹)₂, —(CR⁹R⁹)ₙN₃, —(CR⁹R⁹)ₙNR⁹R⁹, —(CR⁹R⁹)ₙC(O)NR⁹R⁹, —(CR⁹R⁹)ₙC(O)OR⁹ and —(CR⁹R⁹)ₙC(O)R⁹. In certain embodiments of the Syk kinase inhibitors provided herein having the structure of Formula (I), R¹ is a 4-8 membered heterocycloalkyl containing 1 to 2 heteroatoms independently selected from N, O and S, or a 4-8 membered heterocycloalkyl containing 1 to 2 heteroatoms independently selected from N, O and S substituted with 1 to 3 substituents independently selected from hydroxyl and hydroxyl-C₁-C₆alkyl.

In other embodiments of the Syk kinase inhibitors provided herein having the structure of Formula (I), R¹ is selected from

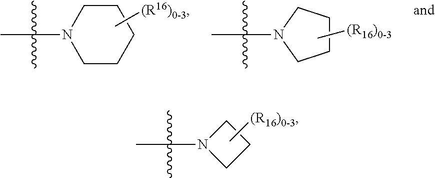

wherein
each R¹⁶ is independently selected from halogen, hydroxyl, hydroxyl-C₁-C₆alkyl, —(CR⁹R⁹)ₙOR⁹, R¹⁰, ═N—OH, —(CR⁹R⁹)ₙSR⁹, —(CR⁹R⁹)ₙOS(O)₂N(R⁹)₂, —(CR⁹R⁹)ₙOS(O)₂N(R⁹)₂, —(CR⁹R⁹)ₙN₃, —(CR⁹R⁹)ₙNR⁹R⁹, —(CR⁹R⁹)ₙC(O)NR⁹R⁹, —(CR⁹R⁹)ₙC(O)OR⁹ and —(CR⁹R⁹)ₙC(O)R⁹, and the Syk kinase inhibitors provided herein are compounds having a structure of Formula (III), and pharmaceutically acceptable salts, pharmaceutically acceptable solvates (e.g. hydrates), the N-oxide derivatives, individual isomers and mixture of isomers thereof:

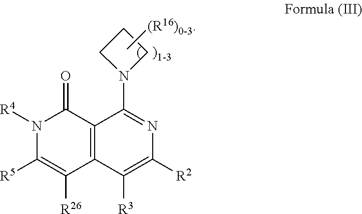

In other embodiments of the Syk kinase inhibitors provided herein having the structure of Formula (I), R¹ is selected from

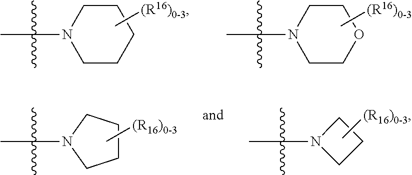

wherein each R¹⁶ is independently selected from hydroxyl and hydroxyl-C₁-C₆alkyl.

In other embodiments of the Syk kinase inhibitors provided herein having the structure of Formula (I), R¹ is an aryl or heteroaryl optionally substituted with 1 to 3 substituents independently selected from halogen, hydroxyl, hydroxyl-C₁-C₆alkyl, —(CR⁹R⁹)ₙOR⁹, R¹⁰, —(CR⁹R⁹)ₙSR⁹, —(CR⁹R⁹)ₙOS(O)₂N(R⁹)₂, —(CR⁹R⁹)ₙOS(O)₂N(R⁹)₂, —(CR⁹R⁹)ₙN₃, —(CR⁹R⁹)ₙNR⁹R⁹, —(CR⁹R⁹)ₙC(O)NR⁹R⁹, —(CR⁹R⁹)ₙC(O)OR⁹ and —(CR⁹R⁹)ₙC(O)R⁹.

In certain embodiments of the Syk kinase inhibitors provided herein having the structure of Formula (I), Formula (II) and Formula (III), R² is R¹⁵, —C(O)R¹², —(CR¹²R¹²)ₙR¹⁴, —CR¹²═NOR¹², C₁-C₆alkyl, C₂-C₆alkene, a C₁-C₆alkyl substituted with 1 to 3 substituents independently selected from —OR¹², —OR¹⁰, —C(O)OR¹², —C(O)R¹⁰, —N(R¹²)₂, —(CR¹²R¹²)ₙR¹⁴, C₁-C₆alkyl and hydroxyl-C₁-C₆alkyl or a C₂-C₆alkene substituted with 1 to 3 substituents independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl.

In certain embodiments of the Syk kinase inhibitors provided herein having the structure of Formula (I), Formula (II) and Formula (III), R² is selected from aryl, heteroaryl and heterocycloalkyl, each of which is optionally substituted with 1 to 3 substituents independently selected from —OR¹², —OR¹⁰, —C(O)OR¹², —C(O)R¹⁰, —N(R¹²)₂, —(CR¹²R¹²)ₙR¹⁴, C₁-C₆alkyl and hydroxyl-C₁-C₆alkyl and each n is independently 0, 1, 2, 3 or 4.

In certain embodiments of the Syk kinase inhibitors provided herein having the structure of Formula (I), Formula (II) and Formula (III), R² is selected from phenyl, a 5, 6, or 9 membered heteroaryl containing 1 to 3 N heteroatoms, and a 4-8 membered heterocycloalkyl containing 1 to 2 heteroatoms independently selected from N, O and S and each n is independently 0, 1, 2, 3 or 4, while in other embodiments R² is selected from phenyl, a 5, 6, or 9 membered heteroaryl containing 1 to 3 N heteroatoms, and a 4-8 membered heterocycloalkyl containing 1 to 2 heteroatoms independently selected from N, O and S each of which is substituted with 1 to 3 substituents independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl and each n is independently 0, 1, 2, 3 or 4.

In certain embodiments of the Syk kinase inhibitors provided herein having the structure of Formula (I), Formula (II) and Formula (III), R² is selected from

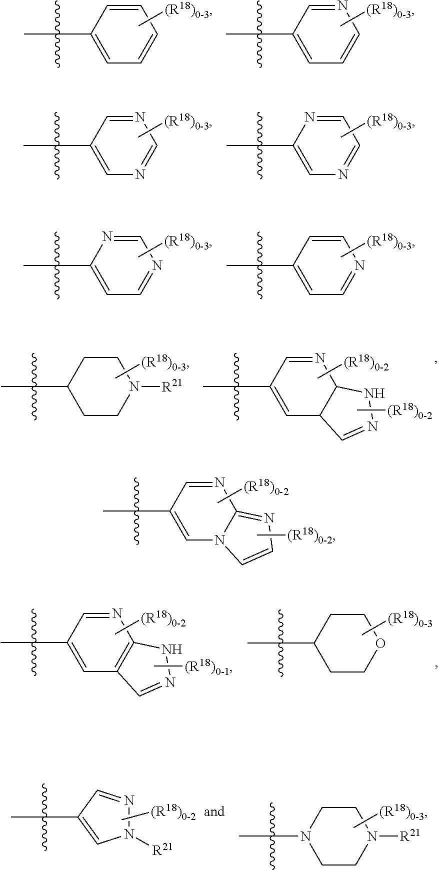

wherein each R¹⁸ is independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl; R¹⁴ is —OR¹², R²¹ is H, C₁-C₆alkyl, —(CR¹²R¹²)₁₋₄R¹⁴ or hydroxyl-C₁-C₆alkyl and each n is independently 0, 1, 2, 3 or 4.

In certain embodiments of the Syk kinase inhibitors provided herein having the structure of Formula (I), Formula (II) and Formula (III), R² is selected from,

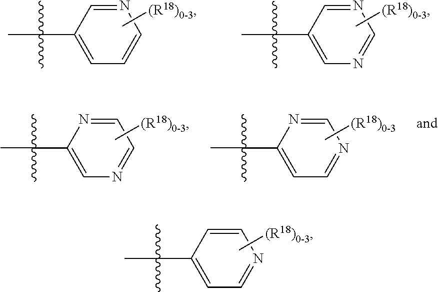

wherein each R¹⁸ independently selected from —OR¹², —C(O)OR¹², —C(O)R¹⁰, —N(R¹²)₂, —(CR¹²R¹²)ₙR¹⁴, —C₁-C₆alkyl and hydroxyl-C₁-C₆alkyl, and each n is independently 0, 1, 2, 3 or 4.

In certain embodiments of the Syk kinase inhibitors provided herein having the structure of Formula (I), Formula (II) and Formula (III), R² is —NR⁸R¹⁰, and the Syk kinase inhibitors provided herein are compounds having a structure of Formula (IV), Formula (V) or Formula (VI), and pharmaceutically acceptable salts, pharmaceutically acceptable solvates (e.g. hydrates), the N-oxide derivatives, individual stereoisomers and mixture of stereoisomers thereof:

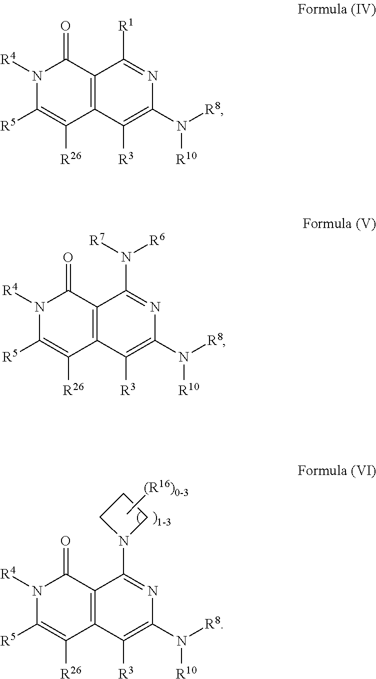

In certain embodiments of the Syk kinase inhibitors provided herein having the structure of Formula (I), Formula (II), Formula (III), Formula (IV), Formula (V) and Formula (VI), R¹⁰ is aryl, heteroaryl, a heteroaryl N-oxide, heterocycloalkyl, C₃-C₈cycloalkyl or —(CR¹²R¹²)ₙR¹¹, wherein the is aryl, heteroaryl, a heteroaryl N-oxide, heterocycloalkyl and C₃-C₈cycloalkyl are each optionally substituted with 1 to 3 substituents independently selected from halogen, hydroxyl, —NO₂, —CN, —C₁-C₆alkyl, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, —OR¹², —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), (CR¹²R¹²)ₙC(O)OR¹², —C(R¹²R¹²R¹⁴), —(CR¹²R¹²)ₙR¹⁴, —O(CR¹²R¹²)₁₋₆R¹⁴, —O(CR¹²R¹²)ₙR¹¹, —(CR³R³)₁₋₆R¹⁴, —(CR¹²R¹²)ₙC(O)NR¹²(CR¹²R¹²)ₙR¹⁴, C(O)NR²⁷(CR¹²R¹²)ₙR¹¹, —(CR¹²R¹²)ₙC(O)NR¹²OR¹², —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, C(R¹²R²⁵R¹³), —C(R¹²R²⁵)(CR¹²R¹²)ₙR¹⁴, CR¹²═CR¹²(CR¹²R¹²)ₙR¹⁴, CR²⁷═N—OR²⁷, C(N(R²⁷)₂)═N—OR²⁷, —CR²⁷═N—O(CR¹²R¹²)₁₋₆R¹⁴, —(CR¹²R¹²)ₙC(O)R¹⁴, —C(O)C(R¹²R¹⁴)(C(R¹²R¹²))ₙR¹⁴, R¹⁵, R¹¹, —C(O)(CR¹²R¹²)ₙR¹⁴, and C(O)C(R¹²R¹²R¹⁴).

In certain embodiments of the Syk kinase inhibitors provided herein having the structure of Formula (I), Formula (II), Formula (III), Formula (IV), Formula (V) and Formula (VI), R¹⁰ is phenyl, a 5, 6 or 9 membered heteroaryl containing 1 to 3 N heteroatoms, an N-oxide of a 5-6 membered heteroaryl containing 1-3 N heteroatoms, a 4-8 membered heterocycloalkyl containing 1 to 2 heteroatoms independently selected from N, O and S, C₃-C₈cycloalkyl or —(CR¹²R¹²)ₙR¹¹, while in other embodiments R¹⁰ is phenyl, a 5, 6 or 9 membered heteroaryl containing 1 to 3 N heteroatoms, an N-oxide of a 5-6 membered heteroaryl containing 1-3 N heteroatoms, a 4-8 membered heterocycloalkyl containing 1 to 2 heteroatoms independently selected from N, O and S, C₃-C₈cycloalkyl each of which is substituted with 1 to 3 substituents independently selected from halogen, hydroxyl, —NO₂, —CN, —C₁-C₆alkyl, —C₂-C₆alkene, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, R¹⁵, R¹¹, —OR¹², —OR¹¹, —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²), —(CR¹²R¹²)ₙC(O)OR¹², —C(R¹²R¹²R¹⁴), —(CR¹²R¹²)ₙR¹⁴, —(CR¹²R¹²)ₙC(O)NR¹²OR¹², —O(CR¹²R¹²)ₙR¹⁴, —O(CR¹²R¹²)ₙR¹¹, —(CR³R³)₁₋₆R¹⁴, —(CR¹²R¹²)ₙC(O)NR¹²(CR¹²R¹²)₁₋₆R¹⁴, —C(O)NR²⁷(CR¹²R¹²)ₙR¹¹, —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₆R¹⁴, —C(O)(CR¹²R¹²)₁₋₆R¹⁴, and —C(O)C(R¹²R¹²R¹⁴).

In certain embodiments of the Syk kinase inhibitors provided herein having the structure of Formula (I), Formula (II), Formula (III), Formula (IV), Formula (V) and Formula (VI), R¹⁰ is selected from

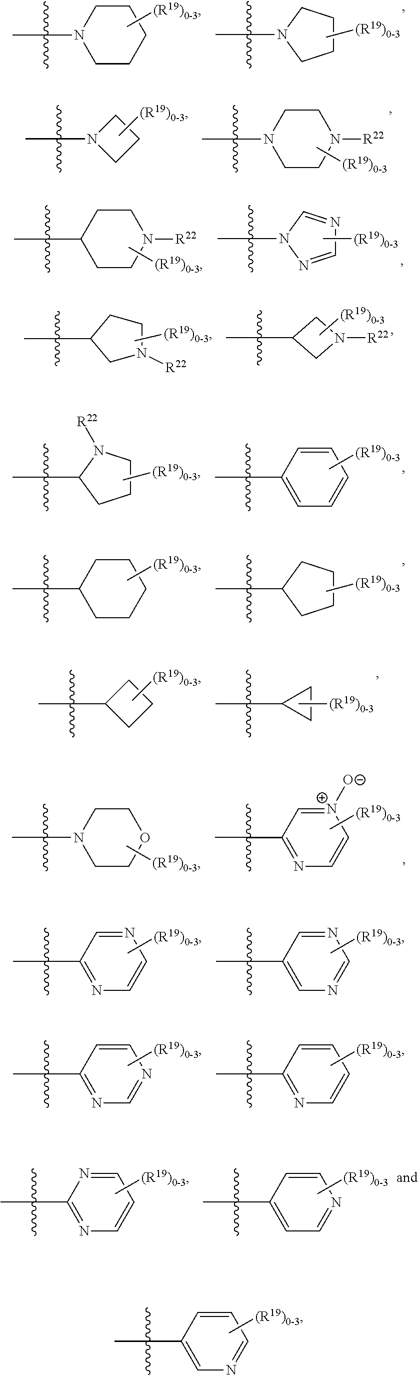

wherein each R¹⁹ is independently selected from halogen, hydroxyl, —NO₂, —CN, —C₁-C₆alkyl, —C₂-C₆alkene, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, R¹⁵, R¹¹, —OR¹², —OR¹¹, —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴, —(CR¹²R¹²)ₙC(O)NR¹²OR¹², —O(CR¹²R¹²)ₙR¹⁴, —O(CR¹²R¹²)ₙR¹¹, —(CR³R³)₁₋₄R¹⁴, —(CR¹²R¹²)ₙC(O)NR¹²(CR¹²R¹²)₁₋₆R¹⁴, —C(O)NR²⁷(CR¹²R¹²)ₙR¹¹, —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₄R¹⁴, —C(O)(CR¹²R¹²)₁₋₄R¹⁴, and —C(O)C(R¹²R¹²R¹⁴); R²² is H, —C₁-C₆alkyl, hydroxyl-C₁-C₆alkyl, —C(O)R¹², —C(O)R¹¹, R¹¹, —C(O)R¹⁵, —(CR¹²R¹²)₁₋₄R¹¹, —(CR¹²R¹²)₁₋₆R¹⁴; R²² is H, hydroxyl, —C₁-C₆alkyl, hydroxyl-C₁-C₆alkyl, —OR¹², —C(O)R¹², —C(O)R¹¹, —C(O)R¹⁵, —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴, —(CR¹²R¹²)ₙC(O)NR¹²(CR¹²R¹²)ₙR¹⁴, —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)R¹⁴, —CO)C(R¹²R¹⁴)(C(R¹²R¹²))ₙR¹⁴, R¹¹, C(O)(CR¹²R¹²)ₙR¹⁴, C(O)C(R¹²R¹²R¹⁴), and

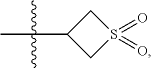

and each n is independently 0, 1, 2, 3 or 4.

In certain embodiments of such R¹⁰ groups, R²² is H, —C₁-C₆alkyl, hydroxyl-C₁-C₆alkyl, —C(O)R¹², —C(O)R¹¹, R¹¹, —C(O)R¹⁵, —(CR¹²R¹²)₁₋₄R¹¹, —(CR¹²R¹²)₁₋₆R¹⁴,

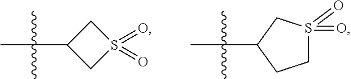

—(CR¹²R¹²)ₙC(O)N(R¹²R¹²), —(CR¹²R¹²)ₙC(O)NR¹²(CR¹²R¹²)₁₋₆R¹⁴, —C(O)C(R¹²R¹⁴)(C(R¹²R¹²))ₙR¹⁴, —(CR¹²R¹²)ₙC(O)NR¹²OR¹², —(CR¹²R¹²)ₙC(R¹²R¹⁴)(C(R¹²R¹²))ₙR¹⁴, —(CR¹²R¹²)ₙC(O)OR¹², —(CR¹²R¹²)ₙC(O)R¹¹ or —(CR¹²R¹²)ₙC(O)(CR¹²R¹²)₁₋₆R¹⁴.

In certain embodiments of the Syk kinase inhibitors provided herein having the structure of Formula (I), Formula (II), Formula (III), Formula (IV), Formula (V) and Formula (VI), R¹⁰ is —(CR¹²R¹²)ₙR¹¹.

In certain embodiments of the Syk kinase inhibitors provided herein having the structure of Formula (I), Formula (II), Formula (III), Formula (IV), Formula (V) and Formula (VI), R¹¹ is a heterocycloalkyl, optionally substituted with 1 to 3 substituents independently selected from halogen, hydroxyl, —C₁-C₆alkyl, C₃-C₈cycloalkyl, hydroxyl-C₁-C₆alkyl and —(CR¹²R¹²)ₙR¹⁴, and each n is independently 0, 1, 2, 3, 4, 5 or 6.

In certain embodiments of the Syk kinase inhibitors provided herein having the structure of Formula (I), Formula (II), Formula (III), Formula (IV), Formula (V) and Formula (VI), R¹¹ is a 4-8 membered heterocycloalkyl containing 1 to 2 heteroatoms independently selected from N, O and S or a 4-8 membered heterocycloalkyl containing 1 to 2 heteroatoms independently selected from N, O and S substituted with 1 to 3 substituents independently selected from halogen, hydroxyl, —C₁-C₆alkyl, C₃-C₈cycloalkyl, hydroxyl-C₁-C₆alkyl and —(CR¹²R¹²)ₙR¹⁴, and each n is independently 0, 1, 2, 3, 4, 5 or 6.

In certain embodiments of the Syk kinase inhibitors provided herein having the structure of Formula (I), Formula (II), Formula (III), Formula (IV), Formula (V) and Formula (VI), R¹¹ is selected from

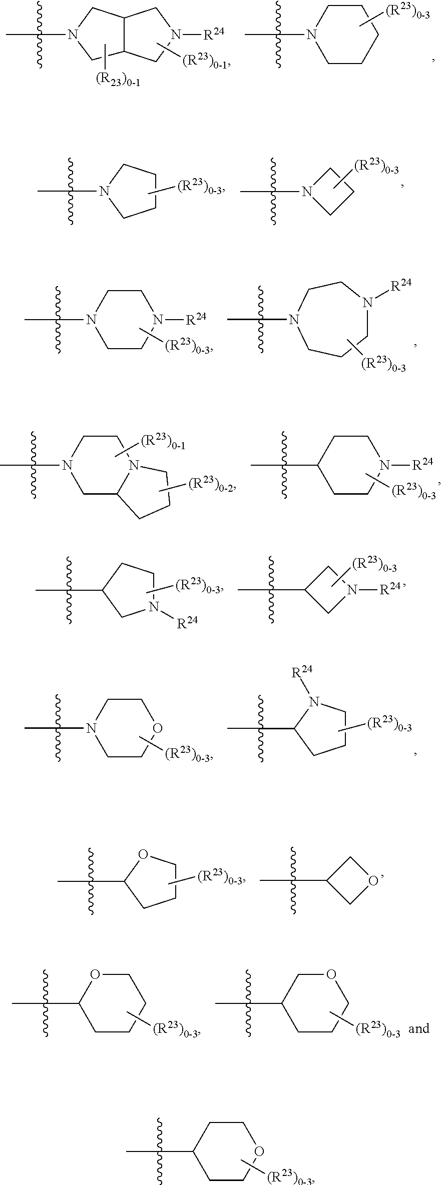

wherein each R²³ is independently selected from halogen, hydroxyl, —C₁-C₆alkyl, C₃-C₈cycloalkyl, hydroxyl-C₁-C₆alkyl and —(CR¹²R¹²)ₙR¹⁴; R²⁴ is H, —C₁-C₆alkyl, C₃-C₈cycloalkyl, hydroxyl-C₁-C₆alkyl or —(CR¹²R¹²)ₙR¹⁴, and each n is independently 0, 1, 2, 3 or 4.

In certain embodiments of the Syk kinase inhibitors provided herein having the structure of Formula (I), Formula (II), Formula (III), Formula (IV), Formula (V) and Formula (VI), is a C₃-C₈cycloalkyl or a C₃-C₈cycloalkyl substituted with 1 to 3 substituents independently selected from halogen, hydroxyl, —C₁-C₆alkyl, C₃-C₈cycloalkyl, hydroxyl-C₁-C₆alkyl and —(CR¹²R¹²)ₙR¹⁴ and each n is independently 0, 1, 2, 3, 4, 5 or 6.

In certain embodiments of the Syk kinase inhibitors provided herein having the structure of Formula (I), Formula (II), Formula (III), Formula (IV), Formula (V) and Formula (VI), R¹¹ is selected from

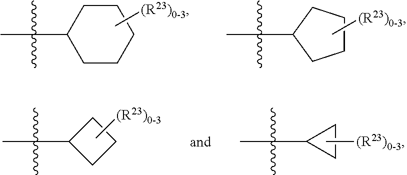

wherein each R²³ is independently selected from halogen, hydroxyl, —C₁-C₆alkyl, C₃-C₈cycloalkyl, hydroxyl-C₁-C₆alkyl and —(CR¹²R¹²)ₙR¹⁴, and each n is independently 0, 1, 2, 3 or 4.

In certain embodiments of the Syk kinase inhibitors provided herein having the structure of Formula (I), Formula (II), Formula (III), Formula (IV), Formula (V) and Formula (VI), R¹¹ is a heteroaryl optionally substituted with 1 to 3 substituents independently selected from halogen, hydroxyl, —C₁-C₆alkyl, C₃-C₈cycloalkyl, hydroxyl-C₁-C₆alkyl and —(CR¹²R¹²)ₙR¹⁴ₙR¹⁴ and each n is independently 0, 1, 2, 3, 4, 5 or 6.

In certain embodiments of the aforementioned compounds of Formula (I), R¹¹ is a 5, 6 or 9 membered heteroaryl containing 1 to 4 heteroatoms independently selected from N, O and S, or a 5, 6 or 9 membered heteroaryl containing 1 to 4 heteroatoms independently selected from N, O and S substituted with 1 to 3 substituents independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴ and each n is independently 0, 1, 2, 3, 4, 5 or 6.

In certain embodiments of the Syk kinase inhibitors provided herein having the structure of Formula (I), Formula (II), Formula (III), Formula (IV), Formula (V) and Formula (VI), R¹¹ is selected from

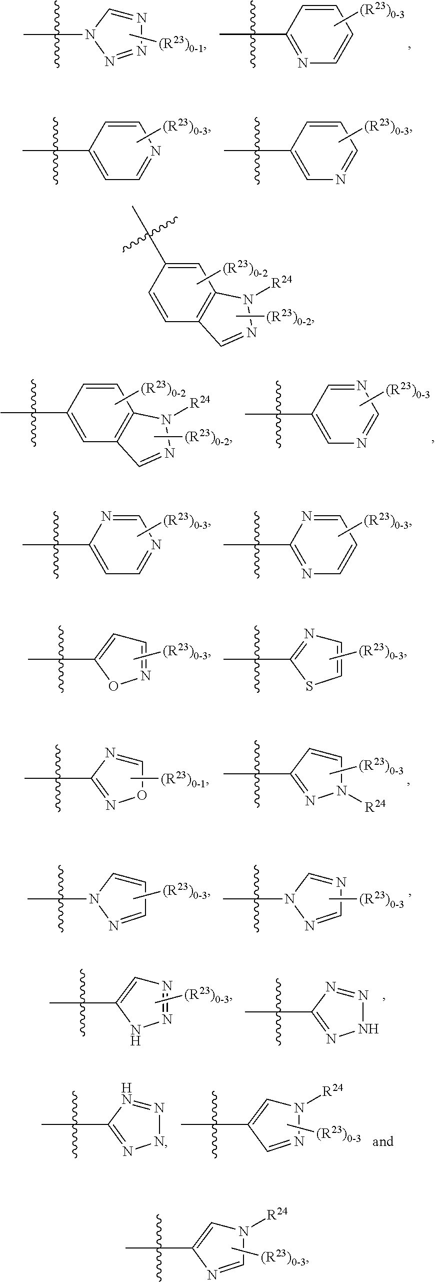

wherein each R²³ is independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴; R²⁴ is H, —C₁-C₆alkyl, C₃-C₈cycloalkyl, hydroxyl-C₁-C₆alkyl or —(CR¹²R¹²)ₙR¹⁴, and each n is independently 0, 1, 2, 3 or 4.

In certain embodiments of the Syk kinase inhibitors provided herein having the structure of Formula (I), Formula (II), Formula (III), Formula (IV), Formula (V) and Formula (VI), R¹¹ is

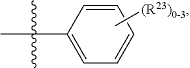

wherein each R²³ is independently selected from halogen, hydroxyl, —C₁-C₆alkyl, C₃-C₈cycloalkyl, hydroxyl-C₁-C₆alkyl and —(CR¹²R¹²)ₙR¹⁴, and each n is independently 0, 1, 2, 3 or 4.

In certain embodiments of the Syk kinase inhibitors provided herein having the structure of Formula (I), Formula (II), Formula (III), Formula (IV), Formula (V) and Formula (VI), R¹⁴ is selected from H, halogen, hydroxyl, hydroxyl-C₁-C₆alkyl, R¹³, —OR¹³, —OR¹², —O(CR¹²R¹²)ₙOR¹³, —C(O)R¹³, —N(R¹²)₂, —NR¹²OR¹³, —CN, —C(O)N(R¹²)₂, —S(O)₂R¹³—C(O)OR¹³, —S(O)₂N(R¹²)₂, —N(R¹²R¹⁰), N(R¹²R¹¹), —(CR¹²R¹²)ₙR¹³, —N(R¹²)(CR¹²R¹²)ₙOR¹³, —C(O)N(R¹²)₂, and R¹⁵. In certain embodiments of the Syk kinase inhibitors provided herein having the structure of Formula (I), Formula (II), Formula (III), Formula (IV), Formula (V) and Formula (VI), R³, R⁵ and R²⁶ are H. In certain embodiments of the Syk kinase inhibitors provided herein having the structure of Formula (I), Formula (II), Formula (III), Formula (IV), Formula (V) and Formula (VI), R⁸ is H. In certain embodiments of the Syk kinase inhibitors provided herein having the structure of Formula (I), Formula (II), Formula (III), Formula (IV), Formula (V) and Formula (VI), R⁴ is H, C₁-C₆alkyl, deuterated C₁-C₆alkyl, C₁-C₆haloalkyl, C₂-C₆alkene, or —CD₃. In certain embodiments of the Syk kinase inhibitors provided herein having the structure of Formula (I), Formula (II), Formula (III), Formula (IV), Formula (V) and Formula (VI), R⁴ is hydroxyl-C₁-C₆alkyl. In certain embodiments of the Syk kinase inhibitors provided herein having the structure of Formula (I), Formula (II), Formula (III), Formula (IV), Formula (V) and Formula (VI), R⁴ is —(CR²⁷R²⁷)₁₋₆R¹⁴, —(CR²⁷R²⁷)(CR²⁷R²⁵)R¹¹, —(CR²⁷R²⁷)(CR²⁷R²⁵)R²⁵, —C(R²⁷R²⁵R²⁵) or —(CR²⁷R²⁷)ₙR¹¹. In certain embodiments of the Syk kinase inhibitors provided herein having the structure of Formula (I), Formula (II), Formula (III), Formula (IV), Formula (V) and Formula (VI), each R²⁵ is independently selected from H, hydroxyl, and hydroxyl-C₁-C₆alkyl.

The present invention also includes all suitable isotopic variations of the compounds provided herein, or pharmaceutically acceptable salts thereof. An isotopic variation of a compound provided herein or a pharmaceutically acceptable salt thereof is defined as one in which at least one atom is replaced by an atom having the same atomic number but an atomic mass different from the atomic mass usually found in nature. Examples of isotopes that may be incorporated into the compounds provided herein and pharmaceutically acceptable salts thereof include but are not limited to isotopes of hydrogen, carbon, nitrogen and oxygen such as ²H, ³H, ¹¹C, ¹³C, ¹⁴C, ¹⁵N, ¹⁷O, ¹⁸O, ³⁵S, ¹⁸F, ³⁶Cl and ¹²³I. Certain isotopic variations of the compounds provided herein and pharmaceutically acceptable salts thereof, for example, those in which a radioactive isotope such as or ³H or ¹⁴C is incorporated, are useful in drug and/or substrate tissue distribution studies. In particular examples, ³H and ¹⁴C isotopes may be used for their ease of preparation and detectability. In other examples, substitution with isotopes such as may afford certain therapeutic advantages resulting from greater metabolic stability, such as increased in vivo half-life or reduced dosage requirements. Isotopic variations of the compounds provided herein or pharmaceutically acceptable salts thereof can generally be prepared by conventional procedures using appropriate isotopic variations of suitable reagents.

The compounds and compositions provided herein are useful for treating or preventing a variety of disorders, including, but not limited to, cytopenias, inflammatory disease, allergic diseases, cell-proliferative diseases, and autoimmune diseased, including, but not limited to, allergic asthma, allergic rhinitis, rheumatoid arthritis, multiple sclerosis, lupus, systemic lupus erythematosus, lymphoma, B cell lymphoma, T cell lymphoma, myelodysplasic syndrome, anemia, leucopenia, neutropenia, thrombocytopenia, granuloctopenia, pancytoia or idiopathic thrombocytopenic purpura.

Certain embodiments of compounds of Formula (I) are useful for treating or preventing a variety of disorders, including, but not limited to, heart disease, diabetes, Alzheimer's disease, immunodeficiency disorders, inflammatory diseases, neurological inflammation, chronic arthritis inflammation, hypertension, respiratory diseases, autoimmune diseases, destructive bone disorders such as osteoporosis, proliferative disorders, infectious diseases, immunologically-mediated diseases, and viral diseases. The compositions are also useful in methods for preventing cell death and hyperplasia and therefore may be used to treat or prevent reperfusion/ischemia in stroke, heart attacks, and organ hypoxia. The compositions are also useful in methods for preventing thrombin-induced platelet aggregation. The compositions are especially useful for disorders such as chronic myelogenous leukemia (CML), acute myeloid leukemia (AML), acute promyelocytic leukemia (APL), rheumatoid arthritis, asthma, osteoarthritis, ischemia, cancer (including, but not limited to, prostate cancer, ovarian cancer, breast cancer and endometrial cancer), liver disease including hepatic ischemia, heart disease such as myocardial infarction and congestive heart failure, pathologic immune conditions involving T cell activation, and neurodegenerative disorders.

### Pharmacology and Utility

Protein kinases (PK) play a central role in the regulation of a wide variety of cellular processes and maintaining control over cellular function. Protein kinases catalyze and regulate the process of phosphorylation, whereby the kinases covalently attach phosphate groups to proteins or lipid targets in response to a variety of extracellular signals. Examples of such stimuli include hormones, neurotransmitters, growth and differentiation factors, cell cycle events, environmental stresses and nutritional stresses. An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events. These diseases include, but are not limited to, autoimmune diseases, inflammatory diseases, bone diseases, metabolic diseases, neurological and neurodegenerative diseases, cancer, cardiovascular diseases, respiratory diseases, allergies and asthma, Alzheimer's disease, and hormone-related diseases.

Examples of protein kinases include, but are not limited to,
(a) tyrosine kinases such as Irk, IGFR-1, Zap-70, Bmx, Btk, CHK (Csk homologous kinase), CSK (C-terminal Src Kinase), Itk-1, Src (c-Src, Lyn, Fyn, Lck, Hck, Yes, Blk, Fgr and Frk), Syk, Tec, Txk/Rlk, Abl, EGFR (EGFR-1/ErbB-1, ErbB-2/NEU/HER-2, ErbB-3 and ErbB-4), FAK, FGF1R (also FGFR1 or FGR-1), FGF2R (also FGR-2), MET (also Met-I or c-MET), PDGFR (.alpha. and .beta.), Tie-1, Tie-2 (also Tek-1 or Tek), VEGFR1 (also FLT-1), VEGFR2 (also KDR), FLT-3, FLT-4, c-KIT, JAK1, JAK2, JAK3, TYK2, LOK, RET, TRKA, PYK2, ALK (Anaplastic Lymphoma Kinase), EPHA (1-8), EPHB (1-6), RON, Fes, Fer or EPHB4 (also EPHB4-1), and
(b) and serine/threonine kinases such as Aurora, c-RAF, SGK, MAP kinases (e.g., MKK4, MKK6, etc.), SAPK2α, SAPK2β, Ark, ATM (1-3), CamK (1-IV), CamKK, Chk1 and 2 (Checkpoint kinases), CK1, CK2, Erk, IKK-I (also IKK-ALPHA or CHUK), IKK-2 (also IKK-BETA), Ilk, Jnk (1-3), LimK (1 and 2), MLK3Raf (A, B, and C), CDK (1-10), PKC (including all PKC subtypes), Plk (1-3), NIK, Pak (1-3), PDK1, PKR, RhoK, RIP, RIP-2, GSK3 (α and β), PKA, P38, Erk (1-3), PKB (including all PKB subtypes) (also AKT-1, AKT-2, AKT-3 or AKT3-1), IRAK1, FRK, SGK, TAK1 and Tp1-2 (also COT).

Phosphorylation modulates or regulates a variety of cellular processes such as proliferation, growth, differentiation, metabolism, apoptosis, motility, transcription, translation and other signaling processes. Aberrant or excessive PTK activity has been observed in many disease states including, but not limited to, benign and malignant proliferative disorders, diseases resulting from inappropriate activation of the immune system and diseases resulting from inappropriate activation of the nervous systems. Specific diseases and disease conditions include, but are not limited to, autoimmune disorders, allograft rejection, graft vs. host disease, diabetic retinopathy, choroidal neovascularization due to age-related macular degeneration, psoriasis, arthritis, osteoarthritis, rheumatoid arthritis, synovial pannus invasion in arthritis, multiple sclerosis, myasthenia gravis, diabetes mellitus, diabetic angiopathy, retinopathy of prematurity, infantile hemangiomas, non-small cell lung, bladder and head and neck cancers, prostate cancer, breast cancer, ovarian cancer, gastric and pancreatic cancer, psoriasis, fibrosis, rheumatoid arthritis, atherosclerosis, restenosis, auto-immune disease, allergy, respiratory diseases, asthma, transplantation rejection, inflammation, thrombosis, retinal vessel proliferation, inflammatory bowel disease, Crohn's disease, ulcerative colitis, bone diseases, transplant or bone marrow transplant rejection, lupus, chronic pancreatitis, cachexia, septic shock, fibroproliferative and differentiative skin diseases or disorders, central nervous system diseases, neurodegenerative diseases, disorders or conditions related to nerve damage and axon degeneration subsequent to a brain or spinal cord injury, acute or chronic cancer, ocular diseases, viral infections, heart disease, lung or pulmonary diseases or kidney or renal diseases and bronchitis.

Tyrosine kinases can be broadly classified as receptor-type (having extracellular, transmembrane and intracellular domains) or the non-receptor type (being wholly intracellular) protein tyrosine kinases. Inappropriate or uncontrolled activation of many of these kinase (aberrant protein tyrosine kinase activity), for example by over-expression or mutation, results in uncontrolled cell growth. Many of the protein tyrosine kinases, whether a receptor or non-receptor tyrosine kinase have been found to be involved in cellular signaling pathways involved in numerous pathogenic conditions, including, but not limited to, immunomodulation, inflammation, or proliferative disorders such as cancer.

Compounds provided herein are inhibitors of Syk kinase activity and as such, the compounds and compositions provided herein are useful for treating diseases or disorders in which Syk kinase contributes to the pathology and/or symptomology of a disease or disorder associated with Syk kinase. Such diseases or disorders include, but are not limited to, lymphomas (by way of example only, B and T cell lymphomas), myelodysplasic syndrome, autoimmune diseases (by way of example only, rheumatoid arthritis and multiple scherosis), cytopenias (by way of example only, anemia, leucopenia, neutropenia, thrombocytopenia, granuloctopenia, pancytoia and idiopathic thrombocytopenic purpura), lupus (by way of example, systemic lupus erythematosus), cancer and allergic disorders (by way of example only, allergic asthma and allergic rhinitis).

In certain embodiments, compounds provided herein are inhibitors of one or more kinases selected from ZAP70, KDR, FMS, FLT3, c-Kit, RET, TrkA, TrkB, TrkC, IGR-1R, Alk and c-FMS kinases, and such compounds are useful for treating diseases or disorders in which ZAP70, KDR, FMS, FLT3, c-Kit, RET, TrkA, TrkB, TrkC, IGR-1R, Alk and c-FMS kinase contributes to the pathology and/or symptomology of a disease or disorder. Non-limiting examples of diseases or disorders associated with ZAP70, KDR, FMS, FLT3, c-Kit, RET, TrkA, TrkB, TrkC, IGR-1R, Alk or c-FMS kinases are provided herein, including, but not limited to, chronic obstructive pulmonary disease (COPD), adult respiratory distress syndrome (ARDS), ulcerative colitis, Crohns disease, bronchitis, dermatitis, psoriasis, scleroderma, urticaria, cancer, breast cancer, HIV, pancreatic cancer, papillary thyroid carcinoma, ovarian carcinoma, human adenoid cystic carcinoma, non small cell lung cancer, secretory breast carcinoma, congenital fibrosarcoma, congenital mesoblastic nephroma, acute myelogenous leukemia, metastasis, cancer-related pain, neuroblastoma, osteosarcoma, melanoma, or a tumor of breast, renal, prostate, colorectal, thyroid, ovarian, pancreatic, neuronal, lung, uterine or gastrointestinal tumor.

### Receptor Tyrosine Kinases (RTKs).

The Receptor Tyrosine Kinases (RTKs) comprise a large family of transmembrane receptors with diverse biological activities. A number of distinct RTK subfamilies have been identified including, but not limited to, EGF receptor family, the Insulin receptor family, the PDGF receptor family, the FGF receptor family, the VEGF receptor family, the HGF receptor family, the Trk receptor family), the EPH receptor family, the AXL receptor family, the LTK receptor family, the TIE receptor family), the ROR receptor family, the DDR receptor family, the RET receptor family, the KLG receptor family, the RYK receptor family and the MuSK receptor family.

Receptor tyrosine kinases have been shown to be not only key regulators of normal cellular processes but also to have a critical role in the development and progression of many types of cancer. The receptor tyrosine kinase (RTK) family includes receptors that are crucial for the growth and differentiation of a variety of cell types. The intrinsic function of RTK mediated signal transduction is initiated by extracellular interaction with a specific growth factor (ligand), typically followed by receptor dimerization, stimulation of the intrinsic protein tyrosine kinase activity and receptor trans-phosphorylation. Binding sites are thereby created for intracellular signal transduction molecules and lead to the formation of complexes with a spectrum of cytoplasmic signaling molecules that facilitate the appropriate cellular response such as, by way of example only, cell division, differentiation, metabolic effects, and changes in the extracellular microenvironment.

Tropomyosin-Receptor-Kinase (Trk) Family

The Trk family receptor tyrosine kinases, TrkA (NTRK1), TrkB (NTRK2), and TrkC (NTRK3), are the signaling receptors that mediate the biological actions of the peptide hormones of the neurotrophin family. Trk receptors are membrane-bound receptor that, through several signal cascades, controls neuronal growth and survival, and differentiation, migration and metastasis of tumor cells. The neurotrophin family of growth factors includes nerve growth factor (NGF), brain-derived neurotrophic factor (BDNF), and two neurotrophins (NT), NT-3, and NT-4. Neurotrophins are critical to the functioning of the nervous system, and the activation of Trk receptors by neurotrophin binding leads to activation of signal cascades resulting in promoting survival and other functional regulation of cells. Each type of neurotrophin has a different binding affinity toward its corresponding Trk receptor, and upon neurotrophin binding, the Trk receptors phosphorylates themselves and members of the MAPK pathway. The differences in the signaling initiated by these distinct types of receptors are important for generating diverse biological responses.

The Trk receptors are implicated in the development and progression of cancer, possibly by upregulation of either the receptor, their ligand (NGF), BDNF, NT-3, and NT-4), or both. In many cases high Trk expression is associated with aggressive tumor behavior, poor prognosis and metastasis. Thus, diseases and disorders related to Trk receptors result from 1) expression of a Trk receptor(s) in cells which normally do not express such a receptor(s); 2) expression of a Trk receptor(s) by cells which normally do not express such a receptor(s); 3) increased expression of Trk receptor(s) leading to unwanted cell proliferation; 4) increased expression of Trk receptor(s) leading to adhesion independent cell survival; 5) mutations leading to constitutive activation of Trk receptor(s); 6) over stimulation of Trk receptor(s) due to abnormally high amount of, or mutations in, Trk receptor(s), and/or 7) abnormally high amount of Trk receptor(s) activity due to abnormally high amount of, or mutations in, Trk receptor(s).

Genetic abnormalities, i.e. point mutations and chromosomal rearrangements involving both the genes expressing TrkB and TrkC have been found in a variety of cancer types. In a kinome-wide approach to identify point mutants in tyrosine kinases, mutations in the genes expressing TrkB and TrkC were found in cell lines and primary samples from patients with colorectal cancer. In addition, chromosomal translocations involving the genes expressing TrkA and TrkB have been found in several different types of tumors. Gene rearrangements involving the genes expressing TrkA and a set of different fusion partners (TPM3, TPR, TFG) are a hallmark of a subset of papillary thyroid cancers. Moreover, secretary breast cancer, infant fibrosarcoma and congenital mesoblastic nephroma have been shown to be associated with a chromosomal rearrangement t(12;15) generating a ETV6-NTRK3 fusion gene that was shown to have constitutive kinase activity and transforming potential in several different cell lines including fibroblasts, hematopoietic cells and breast epithelial cells.

TrkA has the highest affinity to the binding nerve growth factor (NGF). NGF is important in both local and nuclear actions, regulating growth cones, motility, and expression of genes encoding the biosynthesis enzymes for neurotransmitters. Nocireceptive sensory neurons express mostly trkA and not trkB or trkC.

TrkB serves as a receptor for both BDNF and NT-4, and is expressed in neuroendocrine-type cells in the small intestine and the colon, in the alpha cells of the pancreas, in the monocytes and macrophages of the lymph nodes and of the spleen, and in the granular layers of the epidermis. TrkB is also expressed in cancerous prostate cells but not in normal cells.

The binding of BDNF to TrkB receptor causes activation of intercellular cascades which regulate neuronal development and plasticity, long-term potentiation, and apoptosis. BDNF promotes the proliferation, differentiation and growth and survival of normal neural components such as retinal cells and glial cells. In addition, TrkB activation is a potent and specific suppressor of anchorage independent cell death (anoikis), which is apoptosis induced by loss of attachment of a cell to its matrix. By way of example, activation of the Phosphatidylinositol-3kinase/Protein Kinase B signaling axis by TrkB promotes the survival of non-transformed epithelial cells in 3-dimensional cultures and induces tumor formation and metastasis of those cells in immuno-compromised mice. Anchorage independent cell survival is a metastatic process allowing tumor cells to migrate through the systemic circulation and grow at distant organs. Agonism of TrkB results in the failure of induced cell death by cancer treatments. Thus, TrkB modulation is a target for treatment of benign and malignant proliferative diseases, especially tumor diseases.

Diseases and disorders related to the TrkB receptor include, but are not limited to, cancers, such as, by way of example only, neuroblastoma progression, Wilm's tumor progression, breast cancer, pancreatic cancer, colon cancer, prostate cancer, and lung cancer. The TrkB receptor has been shown to be associated with Alzheimer's disease.

TrkC is activated by binding with NT-3 and is expressed by proprioceptive sensory neurons. The axons of these proprioceptive sensory neurons are much thicker than those of nocireceptive sensory neurons, which express TrkA. Signalling through TrkC leads to cell differentiation and development of proprioceptive neurons that sense body position. Mutations in this gene expressing TrkC is associated with medulloblastomas, secretory breast carcinomas and other cancers. In addition, high expression of TrkC is a hallmark of melanoma, especially in cases with brain metastasis.

Certain embodiments of compounds of Formula (I) are also used for the treatment of diseases which respond to an inhibition of the Trk receptor tyrosine kinases (TrkA, TrkB, and TrkC). Certain embodiments of compounds of Formula (I) inhibit Trk receptor tyrosine kinases (TrkA, TrkB, and TrkC) activity and are, therefore, suitable for the treatment of diseases, such as, neuroblastoma, Wilm's tumor, breast cancer, pancreatic cancer, colon cancer, prostate cancer, and lung cancer.

Platelet-Derived Growth Factor (PDGF) Receptor Family

PDGF (Platelet-derived Growth Factor) is a very commonly occurring growth factor, which plays an important role both in normal growth and also in pathological cell proliferation, such as is seen in carcinogenesis and in diseases of the smooth-muscle cells of blood vessels, for example in atherosclerosis and thrombosis. The PDGF growth factor family consists of PDGF-A, PDGF-B, PDGF-C and PDGF-D, which form either homo- or heterodimers (AA, AB, BB, CC, DD) that bind to the protein tyrosine kinase receptors PDGFR-α and PDGFR-β. Dimerization of the growth factors is a prerequisite for activation of the kinase, as the monomeric forms are inactive. The two receptor isoforms dimerize upon binding resulting in three possible receptor combinations, PDGFR-αα, PDGFR-ββ and PDGFR-αβ. Growth factor AA binds only to -αα, growth factor BB can bind with -αα, -ββ and -αβ, growth factors CC and AB specifically interact with -αα and -αβ, and growth factor DD binds to -ββ.

Key downstream mediators of PDGFR signaling are Ras/mitogen-activated protein kinase (MAPK), PI-3 kinase and phospholipase-γ (PLCγ) pathways. MAPK family members regulate various biological functions by phosphorylation of target molecules (transcription factors and other kinases) and thus contribute to regulation of cellular processes such as proliferation, differentiation, apoptosis and immunoresponses. PI-3 kinase activation generated PIPS which functions as a second messenger to activate downstream tyrosine kinases Btk and Itk, the Ser/Thr kinases PDK1 and Akt (PKB). Akt activation is involved in survival, proliferation and cell growth. After activation PLCγ hydrolyses its substrate, PtdIns(4,5)P2, and forms two secondary messengers, diacylglycerol and Ins(1,4,5)P3 which stimulates intracellular processes such as proliferation, angiogenesis and cell motility. The PDGF-receptor plays an important role in the maintenance, growth and development of hematopoietic and non-hematopoietic cells.

PDGFR is expressed on early stem cells, mast cells, myeloid cells, mesenchymal cells and smooth muscle cells. Only PDGFR-β is implicated in myeloid leukemias-usually as a translocation partner with Tel, Huntingtin interacting protein (HIP1) or Rabaptin5. Activation mutations in PDGFR-α kinase domain are associated with gastrointestinal stromal tumors (GIST).

Vascular Endothelial Growth Factor (VEGF) Receptor Family

VEGF, also known as fms-related tyrosine kinase-1 (FLT1), is an important signaling protein involved in both vasculogenesis (formation of embryonic circulatory system) and angiogenesis (growth of blood vessels from pre-existing vasculature). Structurally VEGF belongs to the PDGF family of cytokine-knot growth factors. The VEGF sub-family of growth factors includes VEGF-A, VEGF-B, VEGF-C and VEGF-D. VEGF-A binds to receptor VEGFR-1 (Flt-1) and to VEGFR-2 (KDR/Flk-1). VEGF-C and VEGF-D bind to receptor VEGFR-3 and mediate lymphangiogenesis. The VGFR receptors mediate the angiogenic process, and are thus involved in supporting the progression of cancers and other diseases involving inappropriate vascularization (e.g., diabetic retinopathy, choroidal neovascularization due to age-related macular degeneration, psoriasis, arthritis, retinopathy of prematurity, and infantile hemangiomas).

Fms-Like Tyrosine Kinase

The fms-like tyrosine kinase-3 (FLT3) ligand (FLT3L) is one of the cytokines that affects the development of multiple hematopoietic lineages. These effects occur through the binding of FLT3L to the FLT3 receptor, also referred to as fetal liver tkinase-2 (flk-2) and STK-1, a receptor tyrosine kinase (RTK) expressed on hematopoietic stem and progenitor cells. FLT3 is a member of the type III receptor tyrosine kinase (RTK) family. The ligand for FLT3 is expressed by the marrow stromal cells and other cells and synergizes with other growth factors to stimulate proliferation of stem cells, progenitor cells, dendritic cells, and natural killer cells. Flt3 plays an important role in the maintenance, growth and development of hematopoietic and non-hematopoietic cells.

The FLT3 gene encodes a membrane-bound RTK that plays an important role in proliferation, differentiation and apoptosis of cells during normal hematopoiesis. The FLT3 gene is mainly expressed by early meyloid and lymphoid progenitor cells. Hematopoietic disorders are pre-malignant disorders and include, for instance, the myeloproliferative disorders, such as thrombocythemia, essential thrombocytosis (ET), angiogenic myeloid metaplasia, myelofibrosis (MF), myelofibrosis with myeloid metaplasia (MMM), chronic idiopathic myelofibrosis (IMF), and polycythemia vera (PV), the cytopenias, and pre-malignant myelodysplastic syndromes. Hematological malignancies include leukemias, lymphomas (non-Hodgkin's lymphoma), Hodgkin's disease (also called Hodgkin's lymphoma), and myeloma—for instance, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), multiple myeloma, (MM) and myeloid sarcoma.

Aberrant expression of the Flt3 gene has been documented in both adult and childhood leukemias including acute myeloid leukemia (AML), AML with trilineage myelodysplasia (AML/TMDS), acute lymphoblastic leukemia (ALL), and myelodysplastic syndrome (MDS). Activating mutations of the Flt3 receptor have been found in about 35% of patients with acute myeloblastic leukemia (AML), and are associated with a poor prognosis. The most common mutation involves in-frame duplication within the juxtamembrane domain, with an additional 5-10% of patients having a point mutation at asparagine 835. Both of these mutations are associated with constitutive activation of the tyrosine kinase activity of Flt3, and result in proliferation and viability signals in the absence of ligand. Patients expressing the mutant form of the receptor have been shown to have a decreased chance for cure. Thus, there is accumulating evidence for a role for hyper-activated (mutated) Flt3 kinase activity in human leukemias and myelodysplastic syndrome.

FLT-3 and c-Kit regulate maintenance of stem cell/early progenitor pools as well the development of mature lymphoid and myeloid cells. Both receptors contain an intrinsic kinase domain that is activated upon ligand-mediated dimerization of the receptors. Upon activation, the kinase domain induces autophosphorylation of the receptor as well as the phosphorylation of various cytoplasmic proteins that help propogate the activation signal leading to growth, differentiation and survival. Some of the downstream regulators of FLT-3 and c-Kit receptor signaling include, PLCγ, PI3-kinase, Grb-2, SHIP and Src related kinases. Both receptor tyrosine kinases have been shown to play a role in a variety of hematopoietic and non-hematopoietic malignancies. Mutations that induce ligand independent activation of FLT-3 and c-Kit have been implicated acute-myelogenous leukemia (AML), acute lymphocytic leukemia (ALL), mastocytosis and gastrointestinal stromal tumor (GIST). These mutations include single amino acid changes in the kinase domain or internal tandem duplications, point mutations or in-frame deletions of the juxtamembrane region of the receptors. In addition to activating mutations, ligand dependent (autocrine or paracrine) stimulation of over-expressed wild-type FLT-3 or c-Kit can contribute to the malignant phenotype.

c-Fms encodes for macrophage colony stimulating factor receptor (M-CSF-1R) which is expressed predominately in the monocytes/macrophage lineage. MCSF-1R and its ligand regulate macrophage lineage growth and differentiation. Like the other family members, MCSF-1R contains an intrinsic kinase domain that is activated upon ligand-induced dimerization of the receptor. MCSF-1R is also expressed in non-hematopoietic cells including mammary gland epithelial cells and neurons. Mutations in this receptor are potentially linked to myeloid leukemias and its expression is correlated with metastatic breast, ovarian and endometrial carcinomas. Another possible indication for antagonists of MCSF-1R is osteoporosis.

Certain embodiments of compounds of Formula (I) are inhibitors of FLT-3 and c-kit and are used for the treatment of diseases which respond to an inhibition of the FLT-3 c-kit receptors.

Insulin-Like Growth Factor 1 (IGF-1) Receptor

The Insulin-like Growth Factor 1 (IGF-1) Receptor is a transmembrane receptor that is activated by IGF-1 and by the related growth factor IGF-2. IGF-1R mediates the effects of IGF-1, which is a polypeptide protein hormone similar in molecular structure to insulin. IGF-1 plays an important role in survival and proliferation in mitosis-competent cells, and growth (hypertrophy) in tissues such as skeletal muscle and cardiac muscle. The IGFR signalling pathway is of critical importance during normal development of mammary gland tissue during pregnancy and lactation. During pregnancy, there is intense proliferation of epithelial cells which form the duct and gland tissue. Following weaning, the cells undergo apoptosis and all the tissue is destroyed. Several growth factors and hormones are involved in this overall process, and IGF-1R is believed to have roles in the differentiation of the cells and a key role in inhibiting apoptosis until weaning is complete.

The IGF-1R is implicated in several cancers including, but not limited to, breast cancer. In some instances its anti-apoptotic properties allow cancerous cells to resist the cytotoxic properties of chemotheraputic drugs or radiotherapy. It is further implicated in breast cancer by increasing the metastatic potential of the original tumour by inferring the ability to promote vascularisation.

RET Receptor Family

The RET proto-oncogene encodes a receptor tyrosine kinase for members of the glial cell line derived neurotrophic factor (GDNF) family of extracellular signalling molecules. RET loss of function mutations are associated with the development of Hirschsprung's disease, while gain of function mutaions are associated with development of various types of cancer, including medullar thyroid carcinoma and multiple endocrine neoplasias type II and III.

RET is the receptor for members of the glial cell line derived neurotrophic factor (GDNF) family of extracellular signalling molecules (GFL's). There are three different isoforms, RET51, RET43 and RET9, containing 51, 43 and 9 amino acids in their C-terminal tail, respectively. RET signal transducition is key to the development of normal kidneys and the enteric nervous system.

In order to activate RET GFLs first need to form a complex with a glycosylphosphatidylinositol (GPI)-anchored co-receptor. The co-receptors themselves are classified as members of the GDNF receptor-(GFRα) protein family. Different members of the GFRα family (GFRα1-GFRα4) exhibit a specific binding activity for a specific GFLs. Upon GFL-GFRα complex formation, the complex then brings together two molecules of RET, triggering trans-autophosphorylation of specific tyrosine residues within the tyrosine kinase domain of each RET molecule. Tyr900 and Tyr905 within the activation loop (A-loop) of the kinase domain have been shown to be autophosphorylation sites by mass spectrometry. Phosphorylation of Tyr905 stabilizes the active conformation of the kinase which in turn results in the autophosphorylation of other tyrosine residues mainly located in the C-terminal tail region of the molecule.

c-Kit Receptor

Certain embodiments of compounds of Formula (I) inhibit cellular processes involving stem-cell factor (SCF, also known as the c-kit ligand or steel factor), such as inhibiting SCF receptor (kit) autophosphorylation and SCF-stimulated activation of MAPK kinase (mitogen-activated protein kinase). MO7e cells are a human promegakaryocytic leukemia cell line, which depends on SCF for proliferation.

c-Kit has a substantial homology to the PDGF receptor and to the CSF-1 receptor (c-Fms). Investigations on various erythroid and myeloid cell lines indicate an expression of the c-Kit gene in early stages of differentiation. Certain tumors such as glioblastoma cells likewise exhibit a pronounced expression of the c-Kit gene.

Anaplastic Lymphoma Kinase (Ki-1 or ALK)

ALK is a receptor protein-tyrosine kinase having a putative transmembrane domain and an extracellular domain. ALK plays an important role in the development of the brain and exerts its effects on specific neurons in the nervous system.

Anaplastic lymphoma kinase (ALK), a member of the insulin receptor superfamily of receptor tyrosine kinases, has been implicated in oncogenesis in hematopoietic and non-hematopoietic tumors. The aberrant expression of full-length ALK receptor proteins has been reported in neuroblastomas and glioblastomas; and ALK fusion proteins have occurred in anaplastic large cell lymphoma.

### Non-Receptor Tyrosine Kinases.

Non-receptor tyrosine kinases represent a collection of cellular enzymes that lack extracellular and transmembrane sequences. Over twenty-four individual non-receptor tyrosine kinases, comprising eleven (11) subfamilies (Src, Frk, Btk, Csk, Abl, Zap70, Fes/Fps, Fak, Jak, Ack and LIMK) have been identified. The Src subfamily of non-receptor tyrosine kinases is comprised of the largest number of PTKs and includes Src, Yes, Fyn, Lyn, Lck, Blk, Hck, Fgr and Yrk. The Src subfamily of enzymes has been linked to oncogenesis and immune responses.

The Src family of kinases is implicated in cancer, immune system dysfunction osteopetrosis, and bone remodeling diseases, and therefore Src kinases are considered as potential therapeutic targets for various human diseases. Src expression is linked to cancers such as colon, breast, hepatic and pancreatic cancer, certain B-cell leukemias and lymphomas. In addition, antisense Src expressed in ovarian and colon tumor cells inhibits tumor growth.

Csk, or C-terminal Src kinase, phosphorylates and thereby inhibits Src catalytic activity. Suppression of arthritic bone destruction has been achieved by the overexpression of Csk in rheumatoid synoviocytes and osteoclasts. This implies that Src inhibition may prevent joint destruction that is characteristic in patients suffering from rheumatoid arthritis. Src also plays a role in the replication of hepatitis B virus. The virally encoded transcription factor HBx activates Src in a step required for propagation of the virus.

Other Src family kinases are also potential therapeutic targets. Lck plays a role in T-cell signaling, and mice that lack the Lck gene have a poor ability to develop thymocytes. The function of Lck as a positive activator of T-cell signaling suggests that Lck inhibitors may be useful for treating autoimmune disease such as rheumatoid arthritis. Hck, Fgr and Lyn are important mediators of integrin signaling in myeloid leukocytes. Inhibition of these kinase mediators may therefore be useful for treating inflammation.

Spleen Tyrosine Kinase (Syk)

Spleen tyrosine kinase (Syk) and Zap-70 are members of the Syk family of tyrosine kinases. These non-receptor cytoplasmic tyrosine kinases share a characteristic by a carboxy terminal kinase domain and a dual SH2 domain separated by a linker domain. Syk is a non-receptor linked protein tyrosine kinase which plays a critical role in mediator of immunoreceptor signalling in a host of inflammatory cells including mast cells, B-cells, macrophages and neutrophils. These immunoreceptors, including Fc receptors (such as FcεRI) and the B-cell receptor, are important for both allergic diseases and antibody-mediated autoimmune diseases. Syk also plays a role in FcεRI mediated mast cell degranulation and eosiniphil activation. Accordingly, Syk kinase is implicated in various allergic disorders, in particular asthma.

Inhibition of eosinophil apoptosis has been proposed as key mechanisms for the development of blood and tissue eosinophilia in asthma. IL-5 and GM-CSF are upregulated in asthma and are proposed to cause blood and tissue eosinophilia by inhibition of eosinophil apoptosis. Inhibition of eosinophil apoptosis has been proposed as a key mechanism for the development of blood and tissue eosinophilia in asthma. Syk kinase is required for the prevention of eosinophil apoptosis by cytokines.

While Syk and Zap-70 are primarily expressed in hematopoietic tissues, Syk is also expressed in a variety of other tissues. Within B and T cells respectively, Syk and Zap-70 transmit signals from the B-cell receptor and T-cell receptor. Syk plays a similar role in transmitting signals from a variety of cell surface receptors including CD74, Fc Receptor, and integrins.

Abnormal function of Syk has been implicated in several instances of hematopoeitic malignancies including translocations involving Itk and Tel. Constitutive Syk activity can transform B cells. Several transforming viruses contain “Immunoreceptor Tyrosine Activation Motifs” (ITAMs) which lead to activation of Syk including Epstein Barr virus, bovine leukemia virus, and mouse mammary tumor virus.

Syk kinase is known to play a critical role in other signaling cascades. For example, Syk kinase is an effector of B-cell receptor (BCR) signaling and is an essential component of integrin beta(1), beta(2) and beta(3) signaling in neutrophils.

Syk kinase is important in transducing the downstream cellular signals associated with cross-linking Fc epsilon RI (Fcer1) and or Fc epsilon RI (Fcer1) receptors, and is positioned early in the signalling cascade. In mast cells, for example, the early sequence of Fc epsilon RI (Fcer1) signalling following allergen cross-linking of receptor-IgE complexes involves first Lyn (a Src family tyrosine kinase) and then Syk. Inhibitors of Syk activity would therefore be expected to inhibit all downstream signalling cascades thereby alleviating the immediate allergic response and adverse events initiated by the release of pro-inflammatory mediators and spasmogens.

Allergic rhinitis and asthma are diseases associated with hypersensitivity reactions and inflammatory events involving a multitude of cell types including mast cells, eosinophils, T cells and dendritic cells. Following exposure to allergen, high affinity immunoglobulin receptors for IgE (Fc epsilon RI) and IgG (Fc epsilon.RI) become cross-linked and activate downstream processes in mast cells and other cell types leading to the release of pro-inflammatory mediators and airway spasmogens. In the mast cell, for example, IgE receptor cross-linking by allergen leads to release of mediators including histamine from pre-formed granules, as well as the synthesis and release of newly synthesised lipid mediators including prostaglandins and leukotrienes.

Rheumatoid Arthritis (RA) is an auto-immune disease affecting approximately 1% of the population. It is characterised by inflammation of articular joints leading to debilitating destruction of bone and cartilage. Targeting B cell function is a therapeutic strategy in auto-immune diseases such as RA, with B cell function and auto-antibody production being central to the ongoing pathology in the disease.

Studies using cells from mice deficient in the Spleen Tyrosine Kinase (Syk) have demonstrated a non-redundant role of this kinase in B cell function. The deficiency in Syk is characterised by a block in B cell development. These studies, along with studies on mature B cells deficient in Syk, demonstrate that Syk is required for the differentiation and activation of B cells. Hence, inhibition of Syk in RA patients is likely to block B cell function and thereby reduce Rheumatoid Factor production. In addition to the role of Syk in B cell function, and of further relevance to the treatment of RA, is the requirement for Syk activity in Fc receptor (FcR) signalling. FcR activation by immune complexes in RA has been suggested to contribute to the release of multiple pro-inflammatory mediators.

Syk also plays a role in FcγR dependent and independent response in bone marrow derived macrophages. Syk deficient macrophages are defective in phagocytosis induced by FcγR, but have normal phagocytosis in response to complement. Aerosolized Syk antisense suppresses Syk expression and mediator release from macrophages.

Loss of Syk was found in childhood pro-B cell ALL. Syk is an important suppressor of breast cancer cell growth and metastasis. Tel-Syk fusion protein was found in patients with atypical myelodysplastic syndrome and constitutively activates PI3-K/Akt, MAPK and Jak2 independent STATS signaling. Overexpression of Tel-Syk fusion protein causes B-cell lymphoma in mice (differentiation defect in pre-B-cells). ITK-Syk fusion protein was found in 17% of patients with unspecified peripheral T-cell lymphomas. Syk overexpression is associated with mantle cell lymphoma and Waldenstroem's makroglobulinaemia.

The compounds provided herein are inhibitors of Syk kinase activity and have therapeutic benefit in the treatment of disorders associated with inappropriate Syk activity, in particular in the treatment and prevention of disease states mediated by Syk. Such disease states include cytopenias, inflammatory disease, allergic diseases, cell-proliferative diseases, and autoimmune diseased, including, but not limited to, allergic asthma, allergic rhinitis, rheumatoid arthritis, multiple sclerosis, lupus, systemic lupus erythematosus, lymphoma, B cell lymphoma, T cell lymphoma, myelodysplasic syndrome, anemia, leucopenia, neutropenia, thrombocytopenia, granuloctopenia, pancytoia or idiopathic thrombocytopenic purpura.

Furthermore, the compounds, compositions and methods provided herein include methods of regulating, and in particular inhibiting, signal transduction cascades in which Syk plays a role. The method generally involves contacting a Syk-dependent receptor or a cell expressing a Syk-dependent receptor with an amount of a compound provided herein, or prodrug a compound provided herein, or an acceptable salt, hydrate, solvate, N-oxide and/or composition thereof, effective to regulate or inhibit the signal transduction cascade. The methods are used to regulate, and in particular inhibit, downstream processes or cellular responses elicited by activation of the particular Syk-dependent signal transduction cascade. The methods are practiced to regulate any signal trasduction cascade where Syk is not known or later discovered to play a role. The methods are practiced in in-vitro contexts or in in-vivo contexts as a therapeutic approach towards the treatment or prevention of diseases characterized by, caused by or associated with activation of the Syk-dependent signal transduction cascade. Non-limited examples of such diseases include those provided above.

The compounds and compositions provided herein are inhibitors of Syk kinase, and therefore regulate, and in particular inhibit, any signaling cascade where Syk plays a role, such as, fore example, the Fc receptor, BCR and integrin signaling cascades, as well as the cellular responses elicited through these signaling cascades. The particular cellular response regulated or inhibited will depend, in part, on the specific cell type and receptor signaling cascade. Non-limiting examples of cellular responses that may be regulated or inhibited with the compounds provided herein include a respiratory burst, cellular adhesion, cellular degranulation, cell spreading, cell migration, phagocytosis (e.g., in macrophages), calcium ion flux (e.g., in mast, basophil, neutrophil, eosinophil and B-cells), platelet aggregation, and cell maturation (e.g., in B-cells).

Zeta-Chain-Associated Protein Kinase 70 (ZAP70) Kinase

ZAP-70 is normally expressed in T cells and natural killer cells and has a critical role in the initiation of T-cell signaling. ZAP-70 in B cells is used as a prognostic marker in identifying different forms of chronic lymphocytic leukemia (CLL).

T lymphocytes are activated by engagement of the T cell receptor with processed antigen fragments presented by professional antigen presenting cells (e.g. macrophages, dendritic cells and B cells). Upon this activation, the tyrosine kinase Lck becomes activated and phosphorylates the intracellular portions of the CD3 complex (called ITAMs). The most important member of the CD3 family is CD3-zeta to which ZAP-70 binds. The tandem SH2-domains of ZAP-70 are engaged by the doubly phosphorylated ITAMs of CD3-zeta, which positions ZAP-70 to phosphorylate the transmembrane protein LAT (Linker of Activated T cells). Phosphorylated LAT in turn serves as a docking site to which a number of signaling proteins bind. The final outcome of T cell activation is the transcription of several gene products which allow the T cells to differentiate, proliferate and secrete a number of cytokines.

Certain embodiments of compounds of Formula (I) are inhibitors of ZAP-70 kinase activity and have therapeutic benefit in the treatment of disorders associated with inappropriate ZAP-70 activity, in particular in the treatment and prevention of disease states mediated by ZAP-70.

In accordance with the foregoing, further provided herein are methods for preventing or treating any of the diseases or disorders provided above in a subject in need of such treatment, which method comprises administering to said subject a therapeutically effective amount (See, “Administration and Pharmaceutical Compositions”, infra) of a compound of Formula (I), Formula (II), Formula (III), Formula (IV), Formula (V) or Formula (VI), or a pharmaceutically acceptable salts, pharmaceutically acceptable solvates (e.g. hydrates), the N-oxide derivatives, individual isomers and mixture of isomers thereof. For any of the above uses, the required dosage will vary depending on the mode of administration, the particular condition to be treated and the effect desired.

### Administration and Pharmaceutical Compositions

For the therapeutic uses of compounds provided herein, including compounds of Formulas (I)-(VI), or a pharmaceutically acceptable salts, pharmaceutically acceptable solvates (e.g. hydrates), the N-oxide derivatives, individual isomers and mixture of isomers thereof, such compounds are administered in therapeutically effective amounts either alone or as part of a pharmaceutical composition. Accordingly, provided herein are pharmaceutical compositions, which comprise at least one compound provided herein, including at least one compound of Formulas (I)-(VI), or a pharmaceutically acceptable salts, pharmaceutically acceptable solvates (e.g. hydrates), the N-oxide derivatives, individual isomers and mixture of isomers thereof, and one or more pharmaceutically acceptable carriers, diluents, adjuvant or excipients. In addition, such compounds and compositions are administered singly or in combination with one or more additional therapeutic agents. The method of administration of such compounds and compositions include, but are not limited to, oral administration, rectal administration, parenteral, intravenous administration, intravitreal administration, subcutaneous administration, intramuscular administration, inhalation, intranasal administration, dermal administration, topical administration, ophthalmic administration or buccal administration, tracheal administration, bronchial administration, sublingual administration or otic administration.

The therapeutically effective amount will vary depending on, among others, the disease indicated, the severity of the disease, the age and relative health of the subject, the potency of the compound administered, the mode of administration and the treatment desired. In certain embodiments, the daily dosage of a compound of Formula (I), Formula (II), Formula (III), Formula (IV), Formula (V) or Formula (VI), satisfactory results are indicated to be obtained systemically at daily dosages of from about 0.03 to 2.5 mg/kg per body weight. In certain embodiments, the daily dosage of a compound of Formula (I), Formula (II), Formula (III), Formula (IV), Formula (V) or Formula (VI), administered by inhalation, is in the range from 0.05 micrograms per kilogram body weight (μg/kg) to 100 micrograms per kilogram body weight (μg/kg). In other embodiments, the daily dosage of a compound of Formula (I), Formula (II), Formula (III), Formula (IV), Formula (V) or Formula (VI), administered orally, is in the range from 0.01 micrograms per kilogram body weight (μg/kg) to 100 milligrams per kilogram body weight (mg/kg). An indicated daily dosage in the larger mammal, e.g. humans, is in the range from about 0.5 mg to about 100 mg of a compound of Formula (I), Formula (II), Formula (III), Formula (IV), Formula (V) or Formula (VI), administered, e.g. in divided doses up to four times a day or in controlled release form. In certain embodiment, unit dosage forms for oral administration comprise from about 1 to 50 mg of a compound of Formula (I), Formula (II), Formula (III), Formula (IV), Formula (V) or Formula (VI).

In certain embodiments, compounds provided herein, and pharmaceutically acceptable salts, pharmaceutically acceptable solvates (e.g. hydrates), the N-oxide derivatives, individual isomers and mixture of isomers thereof, are administered as the raw chemical, while in other embodiments the compounds provided herein, and pharmaceutically acceptable salts, pharmaceutically acceptable solvates (e.g. hydrates), the N-oxide derivatives, individual isomers and mixture of isomers thereof, are administered as a pharmaceutical composition. Accordingly, provided herein are pharmaceutical compositions, which comprise at least one compound of Formulas (I), Formula (II) or Formula (III), pharmaceutically acceptable salts, pharmaceutically acceptable solvates (e.g. hydrates), the N-oxide derivatives, individual isomers and mixture of isomers thereof, and one or more pharmaceutically acceptable carriers, diluents, or excipients. Furthermore, another aspect provided herein is a process for the preparation of such pharmaceutical composition including admixing a compound of the Formula (I), Formula (II), Formula (III), Formula (IV), Formula (V) or Formula (VI), provided herein, and pharmaceutically acceptable salts, pharmaceutically acceptable solvates (e.g. hydrates), the N-oxide derivatives, individual isomers and mixture of isomers thereof, with one or more pharmaceutically acceptable carriers, diluents or excipients. Pharmaceutical compositions comprising a compound provided herein in free form or in a pharmaceutically acceptable salt form in association with at least one pharmaceutically acceptable carrier or diluent can be manufactured in a conventional manner by mixing, granulating or coating methods. The compositions may be sterilized and/or contain adjuvants, such as preserving, stabilizing, wetting or emulsifying agents, solution promoters, salts for regulating the osmotic pressure and/or buffers.

Compounds provided herein, and pharmaceutically acceptable salts, pharmaceutically acceptable solvates (e.g. hydrates), the N-oxide derivatives, individual isomers and mixture of isomers thereof, can be administered as pharmaceutical compositions by any conventional route including, but not limited to, intravenous administration (parenteral), oral administration, rectal administration, inhalation, nasal administration, topical administration, ophthalmic administration or otic administration.

Compounds provided herein are administered alone, or are administered by way of known pharmaceutical formulations, including tablets, capsules or elixirs for oral administration, suppositories for rectal administration, sterile solutions or suspensions for parenteral or intramuscular administration, lotions, gels, ointments or creams for topical administration, and the like.

In certain embodiments, pharmaceutical formulations (pharmaceutical compositions) provided herein include those in which the active ingredient is present in at least 1% by weight. In certain embodiments, pharmaceutical formulations (pharmaceutical compositions) provided herein include those in which the active ingredient is present in at least 5% by weight. In certain embodiments, pharmaceutical formulations (pharmaceutical compositions) provided herein include those in which the active ingredient is present in at least 10% by weight. In certain embodiments, pharmaceutical formulations (pharmaceutical compositions) provided herein include those in which the active ingredient is present in at least 20% by weight. In certain embodiments, pharmaceutical formulations (pharmaceutical compositions) provided herein include those in which the active ingredient is present in at least 20% by weight. In certain embodiments, pharmaceutical formulations (pharmaceutical compositions) provided herein include those in which the active ingredient is present in at least 40% by weight. In certain embodiments, pharmaceutical formulations (pharmaceutical compositions) provided herein include those in which the active ingredient is present in at least 50% by weight. That is, the ratio of active ingredient to the other components (by way of example, the addition of adjuvant, diluent and carrier) of the pharmaceutical composition is at least 1:99, 5:95, 10:90, 20:80, 30:70, 40:60 or at least 50:50 by weight.

### Oral Dosage Forms

In certain embodiments, the pharmaceutical compositions containing at least one compound of Formula (I), Formula (II), Formula (III), Formula (IV), Formula (V) or Formula (VI), are administered orally as discrete dosage forms, wherein such dosage forms include, but are not limited to, capsules, gelatin capsules, caplets, tablets, chewable tablets, powders, granules, syrups, flavored syrups, solutions or suspensions in aqueous or non-aqueous liquids, edible foams or whips, and oil-in-water liquid emulsions or water-in-oil liquid emulsions.

The capsules, gelatin capsules, caplets, tablets, chewable tablets, powders or granules, used for the oral administration of at least one compound of Formula (I), Formula (II), Formula (III), Formula (IV), Formula (V) or Formula (VI), are prepared by admixing at least one compound of Formula (I), Formula (II), Formula (III), Formula (IV), Formula (V) or Formula (VI), (active ingredient) together with at least one excipient using conventional pharmaceutical compounding techniques. Non-limiting examples of excipients used in oral dosage forms provided herein include, but are not limited to, binders, fillers, disintegrants, lubricants, absorbents, colorants, flavors, preservatives and sweeteners.

Non-limiting examples of such binders include, but are not limited to, corn starch, potato starch, starch paste, pre-gelatinized starch, or other starches, sugars, gelatin, natural and synthetic gums such as acacia, sodium alginate, alginic acid, other alginates, tragacanth, guar gum, cellulose and its derivatives (by way of example only, ethyl cellulose, cellulose acetate, carboxymethyl cellulose calcium, sodium carboxymethylcellulose, methyl cellulose, hydroxypropyl methylcellulose and microcrystalline cellulose), magnesium aluminum silicate, polyvinyl pyrrolidone and combinations thereof.

Non-limiting examples of such fillers include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein are present in from about 50 to about 99 weight percent of the pharmaceutical composition or dosage form.

Non-limiting examples of such disintegrants include, but are not limited to, agar-agar, alginic acid, sodium alginate, calcium carbonate, sodium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and combinations thereof. In certain embodiments, the amount of disintegrant used in the pharmaceutical compositions provided herein is from about 0.5 to about 15 weight percent of disintegrant, while in other embodiments the amount is from about 1 to about 5 weight percent of disintegrant.

Non-limiting examples of such lubricants include, but are not limited to, sodium stearate, calcium stearate, magnesium stearate, stearic acid, mineral oil, light mineral oil, glycerin, sorbitol, mannitol, polyethylene glycol, other glycols, sodium lauryl sulfate, talc, hydrogenated vegetable oil (by way of example only, peanut oil, cottonseed oil, sunflower oil, sesame oil, olive oil, corn oil, and soybean oil), zinc stearate, sodium oleate, ethyl oleate, ethyl laureate, agar, silica, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.) and combinations thereof. In certain embodiments, the amount of lubricants used in the pharmaceutical compositions provided herein is in an amount of less than about 1 weight percent of the pharmaceutical compositions or dosage forms.

Non-limiting examples of such diluents include, but are not limited to, lactose, dextrose, sucrose, mannitol, sorbitol, cellulose, glycine or combinations thereof.

In certain embodiments, tablets and capsules are prepared by uniformly admixing at least one compound of Formula (I), Formula (II), Formula (III), Formula (IV), Formula (V) or Formula (VI), (active ingredients) with liquid carriers, finely divided solid carriers, or both, and then shaping the product into the desired presentation if necessary. In certain embodiments, tablets are prepared by compression. In other embodiments, tablets are prepared by molding.

In certain embodiments, at least one compound of Formula (I), Formula (II), Formula (III), Formula (IV), Formula (V) or Formula (VI), is orally administered as a controlled release dosage form. Such dosage forms are used to provide slow or controlled-release of one or more compounds of Formula (I), Formula (II), Formula (III), Formula (IV), Formula (V) or Formula (VI). Controlled release is obtained using, for example, hydroxypropylmethyl cellulose, other polymer matrices, gels, permeable membranes, osmotic systems, multilayer coatings, microparticles, liposomes, microspheres, or a combination thereof. In certain embodiments, controlled-release dosage forms are used to extend activity of the compound of Formula (I), Formula (II), Formula (III), Formula (IV), Formula (V) or Formula (VI), dosage frequency, and increase patient compliance.

Administration of compounds of Formula (I), Formula (II), Formula (III), Formula (IV), Formula (V) or Formula (VI), as oral fluids such as solution, syrups and elixirs are prepared in unit dosage forms such that a given quantity of solution, syrups or elixirs contains a predetermined amount of a compound of Formula (I), Formula (II), Formula (III), Formula (IV), Formula (V) or Formula (VI). Syrups are prepared by dissolving the compound in a suitably flavored aqueous solution, while elixirs are prepared through the use of a non-toxic alcoholic vehicle. Suspensions are formulated by dispersing the compound in a non-toxic vehicle. Non-limiting examples of excipients used in as oral fluids for oral administration include, but are not limited to, solubilizers, emulsifiers, flavoring agents, preservatives, and coloring agents. Non-limiting examples of solubilizers and emulsifiers include, but are not limited to, water, glycols, oils, alcohols, ethoxylated isostearyl alcohols and polyoxy ethylene sorbitol ethers. Non-limiting examples of preservatives include, but are not limited to, sodium benzoate. Non-limiting examples of flavoring agents include, but are not limited to, peppermint oil or natural sweeteners or saccharin or other artificial sweeteners.

### Parenteral Dosage Forms

In certain embodiments pharmaceutical compositions containing at least one compound of Formula (I), Formula (II), Formula (III), Formula (IV), Formula (V) or Formula (VI), are administered parenterally by various routes including, but not limited to, subcutaneous, intravenous (including bolus injection), intramuscular, and intraarterial.

Such parenteral dosage forms are administered in the form of sterile or sterilizable injectable solutions, suspensions, dry and/or lyophylized products ready to be dissolved or suspended in a pharmaceutically acceptable vehicle for injection (reconstitutable powders) and emulsions. Vehicles used in such dosage forms include, but are not limited to, Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### Transdermal Dosage Forms

In certain embodiments pharmaceutical compositions containing at least one compound of Formula (I), Formula (II), Formula (III), Formula (IV), Formula (V) or Formula (VI), are administered transdemally. Such transdermal dosage forms include “reservoir type” or “matrix type” patches, which are applied to the skin and worn for a specific period of time to permit the penetration of a desired amount of a compound of Formula (I), Formula (II), Formula (III), Formula (IV), Formula (V) or Formula (VI). By way of example only, such transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. In other embodiments, matrix transdermal formulations are used.

Formulations for transdermal delivery of a compound of Formula (I), Formula (II), Formula (III), Formula (IV), Formula (V) or Formula (VI), include an effective amount of a compound of Formula (I), Formula (II), Formula (III), Formula (IV), Formula (V) or Formula (VI), a carrier and an optional diluent. A carrier includes, but is not limited to, absorbable pharmacologically acceptable solvents to assist passage through the skin of the host, such as water, acetone, ethanol, ethylene glycol, propylene glycol, butane-1,3-diol, isopropyl myristate, isopropyl palmitate, mineral oil, and combinations thereof.

In certain embodiments, such transdermal delivery systems include penetration enhancers to assist in delivering one or more compounds of Formula (I), Formula (II), Formula (III), Formula (IV), Formula (V) or Formula (VI), to the tissue. Such penetration enhancers include, but are not limited to, acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

In other embodiments, the pH of such a transdermal pharmaceutical composition or dosage form, or of the tissue to which the pharmaceutical composition or dosage form is applied, is adjusted to improve delivery of one or more compounds of Formula (I), Formula (II), Formula (III), Formula (IV), Formula (V) or Formula (VI). In other embodiments, the polarity of a solvent carrier, its ionic strength, or tonicity are adjusted to improve delivery. In other embodiments, compounds such as stearates are added to advantageously alter the hydrophilicity or lipophilicity of one or more compounds of Formula (I), Formula (II), Formula (III), Formula (IV), Formula (V) or Formula (VI), so as to improve delivery. In certain embodiments, such stearates serve as a lipid vehicle for the formulation, as an emulsifying agent or surfactant, and as a delivery-enhancing or penetration-enhancing agent. In other embodiments, different salts, hydrates or solvates of the compounds of Formula (I), Formula (II), Formula (III), Formula (IV), Formula (V) or Formula (VI), are used to further adjust the properties of the resulting composition.

### Topical Dosage Forms

In certain embodiments at least one compound of Formula (I), Formula (II), Formula (III), Formula (IV), Formula (V) or Formula (VI), is administered by topical application of pharmaceutical composition containing at least one compound of Formula (I), Formula (II), Formula (III), Formula (IV), Formula (V) or Formula (VI), in the form of lotions, gels, ointments solutions, emulsions, suspensions or creams. Suitable formulations for topical application to the skin are aqueous solutions, ointments, creams or gels, while formulations for ophthalmic administration are aqueous solutions. Such formulations optionally contain solubilizers, stabilizers, tonicity enhancing agents, buffers and preservatives.

Such topical formulations include at least one carrier, and optionally at least one diluent. Such carriers and diluents include, but are not limited to, water, acetone, ethanol, ethylene glycol, propylene glycol, butane-1,3-diol, isopropyl myristate, isopropyl palmitate, mineral oil, and combinations thereof.

In certain embodiments, such topical formulations include penetration enhancers to assist in delivering one or more compounds of Formula (I), Formula (II), Formula (III), Formula (IV), Formula (V) or Formula (VI), to the tissue. Such penetration enhancers include, but are not limited to, acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

In certain embodiments pharmaceutical compositions containing at least one compound of Formula (I), Formula (II), Formula (III), Formula (IV), Formula (V) or Formula (VI), are administered by inhalation. Dosage forms for inhaled administration are formulated as aerosols or dry powders. Aerosol formulations for inhalation administration comprise a solution or fine suspension of at least one compound of Formula (I), Formula (II), Formula (III), Formula (IV), Formula (V) or Formula (VI), in a pharmaceutically acceptable aqueous or non-aqueous solvent. In addition, such pharmaceutical compositions optionally comprise a powder base such as lactose, glucose, trehalose, mannitol or starch, and optionally a performance modifier such as L-leucine or another amino acid, and/or metals salts of stearic acid such as magnesium or calcium stearate.

In certain embodiments, compounds of Formula (I), Formula (II), Formula (III), Formula (IV), Formula (V) or Formula (VI), are be administered directly to the lung by inhalation using a Metered Dose Inhaler (“MDI”), which utilizes canisters that contain a suitable low boiling propellant, e.g., dichlorodifluoromethane, trichlorofluoromethane, dichlorotetrafluoroethane, carbon dioxide or other suitable gas, or a Dry Powder Inhaler (DPI) device which uses a burst of gas to create a cloud of dry powder inside a container, which is then be inhaled by the patient. In certain embodiments, capsules and cartridges of gelatin for use in an inhaler or insufflator are formulated containing a powder mixture of a compound of Formula (I), Formula (II), Formula (III), Formula (IV), Formula (V) or Formula (VI), and a powder base such as lactose or starch. In certain embodiments, compounds of Formula (I), Formula (II), Formula (III), Formula (IV), Formula (V) or Formula (VI), are delivered to the lung using a liquid spray device, wherein such devices use extremely small nozzle holes to aerosolize liquid drug formulations that can then be directly inhaled into the lung. In other embodiments, compounds of Formula (I), Formula (II), Formula (III), Formula (IV), Formula (V) or Formula (VI), are delivered to the lung using a nebulizer device, wherein a nebulizers creates an aerosols of liquid drug formulations by using ultrasonic energy to form fine particles that can be readily inhaled. In other embodiments, compounds of Formula (I), Formula (II), Formula (III), Formula (IV), Formula (V) or Formula (VI), are delivered to the lung using an electrohydrodynamic (“EHD”) aerosol device wherein such EHD aerosol devices use electrical energy to aerosolize liquid drug solutions or suspensions.

In certain embodiments, the pharmaceutical composition containing at least one compound of Formula (I), Formula (II), Formula (III), Formula (IV), Formula (V) or Formula (VI), or pharmaceutically acceptable salts and solvates thereof, provided herein, also contain one or more absorption enhancers. In certain embodiments, such absorption enhancers include, but are not limited to, sodium glycocholate, sodium caprate, N-lauryl-β-D-maltopyranoside, EDTA, and mixed micelles.

In certain embodiments pharmaceutical compositions containing at least one compound of Formula (I), Formula (II), Formula (III), Formula (IV), Formula (V) or Formula (VI), are administered nasally. The dosage forms for nasal administration are formulated as aerosols, solutions, drops, gels or dry powders.

In certain embodiments pharmaceutical compositions containing at least one compound of Formula (I), Formula (II), Formula (III), Formula (IV), Formula (V) or Formula (VI), are administered rectally in the form of suppositories, enemas, ointment, creams rectal foams or rectal gels. In certain embodiments such suppositories are prepared from fatty emulsions or suspensions, cocoa butter or other glycerides.

In certain embodiments pharmaceutical compositions containing at least one compound of Formula (I), Formula (II), Formula (III), Formula (IV), Formula (V) or Formula (VI), are administered opthamically as eye drops. Such formulations are aqueous solutions that optionally contain solubilizers, stabilizers, tonicity enhancing agents, buffers and preservatives.

In certain embodiments pharmaceutical compositions containing at least one compound of Formula (I), Formula (II), Formula (III), Formula (IV), Formula (V) or Formula (VI), are administered otically as ear drops. Such formulations are aqueous solutions that optionally contain solubilizers, stabilizers, tonicity enhancing agents, buffers and preservatives.

In certain embodiments pharmaceutical compositions containing at least one compound of Formula (I), Formula (II), Formula (III), Formula (IV), Formula (V) or Formula (VI), are formulated as a depot preparation. Such long acting formulations are administered by implantation (for example subcutaneously or intramuscularly) or by intramuscular injection. In certain embodiments, such formulations include polymeric or hydrophobic materials (for example, as an emulsion in an acceptable oil) or ion exchange resins, or as sparingly soluble derivatives, for example, as a sparingly soluble salt.

In other embodiments, a compound of Formula (I), Formula (II), Formula (III), Formula (IV), Formula (V) or Formula (VI), provided herein, or a pharmaceutically acceptable salt, N-oxide, isomer or solvate thereof, or a pharmaceutical composition containing such compounds of Formula (I), Formula (II), Formula (III), Formula (IV), Formula (V) or Formula (VI), is administered to a patient who has not previously undergone or is not currently undergoing treatment with another therapeutic agent.

In certain embodiments pharmaceutical compositions containing at least one compound of Formula (I), Formula (II), Formula (III), Formula (IV), Formula (V) or Formula (VI), are administered in the form of liposome delivery systems, such as small unilamellar vesicles, large unilamellar vesicles and multilamellar vesicles. Liposomes are formed from cholesterol, stearylamine, or a variety of phospholipids, such as phosphatidylcholines.

Provided herein are compounds of Formula (I), Formula (II), Formula (III), Formula (IV), Formula (V) or Formula (VI), and salts, solvates and pharmaceutical compositions thereof for use in modulating Syk activity, including inhibiting Syk activity. Provided herein are compounds of Formula (I), Formula (II), Formula (III), Formula (IV), Formula (V) or Formula (VI), and salts, solvates and pharmaceutical compositions thereof for use in the treatment of diseases and conditions mediated by inappropriate Syk activity. By way of example only, this inappropriate Syk activity is any Syk activity that deviates from the normal Syk activity expected in a particular mammalian subject. Inappropriate Syk activity may take the form of, for instance, an abnormal increase in activity, or an aberration in the timing and or control of Syk activity. Such inappropriate activity may result then, for example, from overexpression or mutation of the protein kinase leading to inappropriate or uncontrolled activation.

In a further embodiment, the present invention is directed to methods of regulating, modulating, or inhibiting Syk, using compounds of Formula (I), Formula (II), Formula (III), Formula (IV), Formula (V) or Formula (VI), or a pharmaceutically acceptable salt or solvate thereof, for the prevention and/or treatment of disorders related to unregulated Syk activity.

In a further embodiment, the present invention provides a method of treatment of a mammal suffering from a disorder mediated by Syk activity, which includes administering to said subject an effective amount of a compound of Formula (I), Formula (II), Formula (III), Formula (IV), Formula (V) or Formula (VI), or a pharmaceutically acceptable salt, solvate, or a physiologically functional derivative thereof.

In a further embodiment, the present invention provides for the use of a compound of Formula (I), Formula (II), Formula (III), Formula (IV), Formula (V) or Formula (VI), or a pharmaceutically acceptable salt or solvate thereof, in the preparation of a medicament for the treatment of a disease or condition/disorder mediated by Syk activity.

In a further embodiment, the disease or condition mediated by inappropriate Syk activity is rheumatoid arthritis. In a further embodiment, the disease or condition mediated by inappropriate Syk activity is allergic rhinitis. In a further embodiment, the disease or condition mediated by inappropriate Syk activity is rheumatoid arthritis. In a further embodiment, the disease or condition mediated by inappropriate Syk activity is asthma or allergic rhinitis. In a further embodiment, the disease or condition mediated by inappropriate Syk activity is lymphoma.

In a further embodiment, the present invention provides a pharmaceutical composition comprising at least one compound of Formula (I), Formula (II), Formula (III), Formula (IV), Formula (V) or Formula (VI), adapted for administration by the oral route, for treating, for example, rheumatoid arthritis. In a further embodiment, the present invention provides a pharmaceutical composition comprising at least one compound of Formula (I), Formula (II), Formula (III), Formula (IV), Formula (V) or Formula (VI), adapted for administration by the nasal route, for treating, for example, allergic rhinitis. In a further embodiment, the present invention provides a pharmaceutical composition comprising at least one compound of Formula (I), Formula (II), Formula (III), Formula (IV), Formula (V) or Formula (VI), adapted for administration by the inhaled route, for treating, for example, asthma or allergic rhinitis.

Combination Therapies

In certain embodiments, a compound of Formula (I), Formula (II), Formula (III), Formula (IV), Formula (V) or Formula (VI), is used in combination with a second therapeutic agent, for ameliorating a condition mediated by a protein kinase, such as a Syk-mediated condition. In certain embodiments, the compounds provided herein are used in combination with a chemotherapeutic agent to treat a cell proliferative disorder, including but not limited to, lymphoma, osteosarcoma, melanoma, or a tumor of breast, renal, prostate, colorectal, thyroid, ovarian, pancreatic, neuronal, lung, uterine or gastrointestinal tumor. In certain embodiments, the compounds provided herein are used in combination with an agent to treat respiratory diseases.

In certain embodiments, compounds of the present invention, and their salts and solvates thereof, are administered alone or in combination with other therapeutic agents (pharmaceutical combinations) for the treatment of diseases and conditions associated with inappropriate Syk activity. In certain embodiment, the compounds and pharmaceutically acceptable compositions provided herein are administered concurrently with one or more other desired therapeutics or medical procedures. In other embodiment, the compounds and pharmaceutically acceptable compositions provided herein are administered prior to one or more other desired therapeutics or medical procedures. In certain embodiment, the compounds and pharmaceutically acceptable compositions provided herein are administered subsequent to one or more other desired therapeutics or medical procedures.

Chemotherapeutic agents or other anti-proliferative agents used in combination with the compounds provided herein to treat proliferative diseases and cancer include, but are not limited to, surgery, radiotherapy (gamma.-radiation, neutron beam radiotherapy, electron beam radiotherapy, proton therapy, brachytherapy, and systemic radioactive isotopes), endocrine therapy, biologic response modifiers (interferons, interleukins, and tumor necrosis factor (TNF)), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabine, Gemcitabine), spindle poisons (Vinblastine, Vincristine, Vinorelbine, Paclitaxel), podophyllotoxins, camptothecin, camptothecin analogs (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), GLEEVEC™, adriamycin and dexamethasone.

Other chemotherapeutic agents which are used in the compositions and methods provided herein include but are not limited to anthracyclines, alkylating agents (e.g., mitomycin C), alkyl sulfonates, aziridines, ethylenimines, methylmelamines, nitrogen mustards, nitrosoureas, antibiotics, antimetabolites, folic acid analogs (e.g., dihydrofolate reductase inhibitors such as methotrexate), purine analogs, pyrimidine analogs, enzymes, podophyllotoxins, platinum-containing agents, interferons, and interleukins Particular examples of known chemotherapeutic agents which may be used in the compositions and methods provided herein include, but are not limited to, busulfan, improsulfan, piposulfan, benzodepa, carboquone, meturedepa, uredepa, altretamine, triethylenemelamine, triethylenephosphoramide, triethylenethiophosphoramide, trimethylolomelamine, chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, doxorubicin, epirubicin, mitomycin C, mycophenolic acid, nogalamycin, olivomycin, peplomycin, plicamycin, porfiromycin, puromycin, streptonigrin, streptozocin, tubercidin, ubenimex, zinostatin, zorubicin, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, fluorouracil, tegafur, L-asparaginase, pulmozyme, aceglatone, aldophosphamide glycoside, aminolevulinic acid, amsacrine, bestrabucil, bisantrene, carboplatin, cisplatin, defofamide, demecolcine, diaziquone, elformithine, elliptinium acetate, etoglucid, etoposide, flutamide, gallium nitrate, hydroxyurea, interferon-alpha, interferon-beta, interferon-gamma, interleukin-2, lentinan, lonidamine, mitoguazone, mitoxantrone, mopidamol, nitracrine, pentostatin, phenamet, pirarubicin, podophyllinic acid, 2-ethylhydrazide, procarbazine, razoxane, sizofuran, spirogermanium, paclitaxel, tamoxifen, teniposide, tenuazonic acid, triaziquone, 2,2′,2″-trichlorotriethylamine, urethane, vinblastine, vincristine, and vindesine.

Other agents used in combination with the compounds provided herein include, but are not limited to: treatments for Alzheimer's Disease such as ARRICEPT™ and EXCELON™; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., AVONEX™ and REBIF™), COPAXONE™, and mitoxantrone; treatments for asthma such as albuterol and SINGULAIR™; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

Other agents having synergic effects when used in combination with the compounds include, but are not limited to, immunomodulatory or anti-inflammatory substances, for example cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where the compounds provided herein are administered in conjunction with other therapies, dosages of the co-administered compounds will of course vary depending on the type of co-drug employed, on the specific drug employed, on the condition being treated and so forth.

Also provided herein are pharmaceutical combinations, e.g. a kit, comprising a) a first agent which is a compound provided herein as disclosed herein, in free form or in pharmaceutically acceptable salt form, and b) at least one co-agent. The kit can comprise instructions for its administration.

### Processes for Making Compounds of the Invention

General procedures for preparing compounds provided herein are provided in the Examples, infra. In the reactions provided, reactive functional groups, for example hydroxy, amino, imino, thio or carboxy groups, where these are desired in the final product, may be protected to avoid their unwanted participation in the reactions. Conventional protecting groups may be used in accordance with standard practice (see e.g., T. W. Greene and P. G. M. Wuts in “Protective Groups in Organic Chemistry”, John Wiley and Sons, 1991).

In certain embodiments compounds provided herein are prepared as a pharmaceutically acceptable acid addition salt by reacting the free base form of the compound with a pharmaceutically acceptable inorganic or organic acid. In other embodiments, a pharmaceutically acceptable base addition salt of a compound provided herein is prepared by reacting the free acid form of the compound with a pharmaceutically acceptable inorganic or organic base. Alternatively, the salt forms of the compounds provided herein are prepared using salts of the starting materials or intermediates. In certain embodiments, the compounds provided herein are in the form of other salts including, but not limited to, oxalates or trifluoroacetates.

A pharmaceutically acceptable acid addition salt is formed by reaction of the free base form a compound of Formula (I), Formula (II), Formula (III), Formula (IV), Formula (V) or Formula (VI), with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid. A pharmaceutically acceptable acid addition salt of a compound of formula (I) can comprise or be, for example, a hydrobromide, hydrochloride, sulfate, nitrate, phosphate, succinate, maleate, formarate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

The free acid or free base forms of the compounds provided herein may be prepared from the corresponding base addition salt or acid addition salt from, respectively. For example a compound provided herein in an acid addition salt form may be converted to the corresponding free base by treating with a suitable base (e.g., ammonium hydroxide solution, sodium hydroxide, and the like). A compound provided herein in a base addition salt form may be converted to the corresponding free acid by treating with a suitable acid (e.g., hydrochloric acid, etc.).

Compounds provided herein in unoxidized form may be prepared from N-oxides of compounds provided herein by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds provided herein may be prepared by methods known to those of ordinary skill in the art (e.g., for further details see Saulnier et al., (1994), Bioorganic and Medicinal Chemistry Letters, Vol. 4, p. 1985). For example, appropriate prodrugs may be prepared by reacting a non-derivatized compound provided herein with a suitable carbamylating agent (e.g., 1,1-acyloxyalkylcarbanochloridate, para-nitrophenyl carbonate, or the like).

Protected derivatives of the compounds provided herein may be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention may be conveniently prepared or formed during the process provided herein, as solvates (e.g., hydrates). Hydrates of compounds of the present invention may be conveniently prepared by recrystallization from an aqueous/organic solvent mixture, using organic solvents such as dioxin, tetrahydrofuran or methanol.

Compounds provided herein may be prepared as their individual stereoisomers by reacting a racemic mixture of the compound with an optically active resolving agent to form a pair of diastereoisomeric compounds, separating the diastereomers and recovering the optically pure enantiomers. Resolution of enantiomers may be carried out using covalent diastereomeric derivatives of the compounds provided herein, or by using dissociable complexes (e.g., crystalline diastereomeric salts). Diastereomers have distinct physical properties (e.g., melting points, boiling points, solubility, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

Compounds of Formula (I) are made by processes provided herein and in the Examples. In certain embodiments, compounds of Formula (I) are made by:
(a) optionally converting a compound provided herein into a pharmaceutically acceptable salt;
(c) optionally converting a salt form of a compound provided herein to a non-salt form;
(d) optionally converting an unoxidized form of a compound provided herein into a pharmaceutically acceptable N-oxide;
(e) optionally converting an N-oxide form of a compound provided herein to its unoxidized form;
(f) optionally resolving an individual isomer of a compound provided herein from a mixture of isomers;
(g) optionally converting a non-derivatized compound provided herein into a pharmaceutically acceptable prodrug derivative; and
(h) optionally converting a prodrug derivative of a compound provided herein to its non-derivatized form.

One of skill in the art will appreciate that the above transformations are only representative of methods for preparation of the compounds of the present invention, and that other well known methods can similarly be used.

Certain methods for the synthesis of compounds of Formula (I) is provided in reaction schemes (I)-(XII), wherein schemes (I)-(VI) illustrate the synthesis of intermediates used to make compounds of Formula (I), and schemes (VII)-(XII) illustrate the use of these intermediates to make certain compounds of Formula (I).

The synthesis of certain compounds of Formula (I) is illustrated in scheme (I), scheme (II), scheme (III) and scheme (IV).

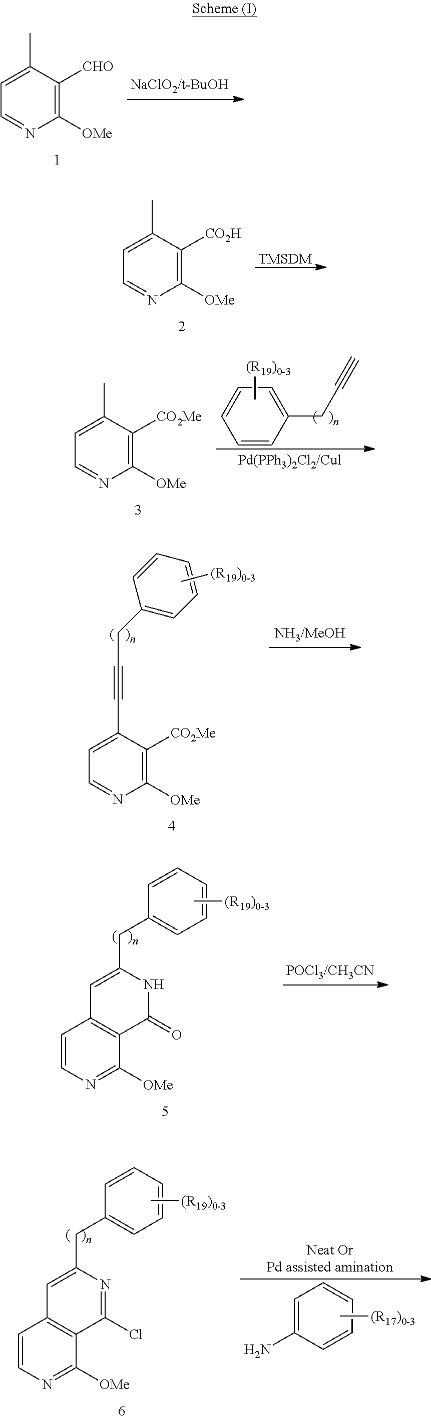

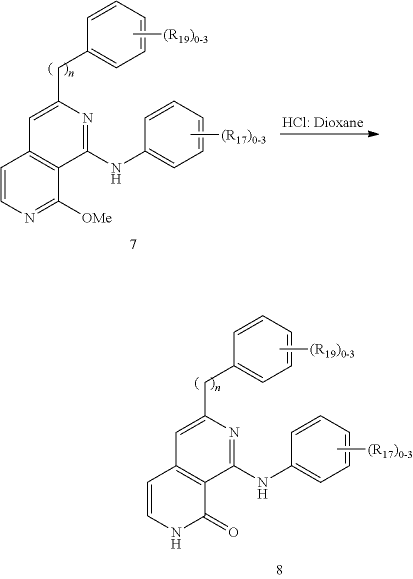

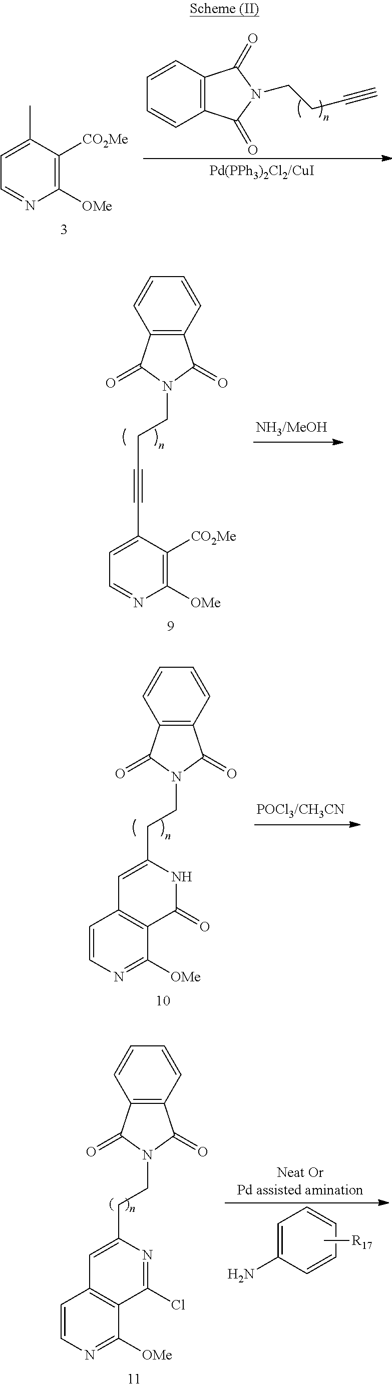

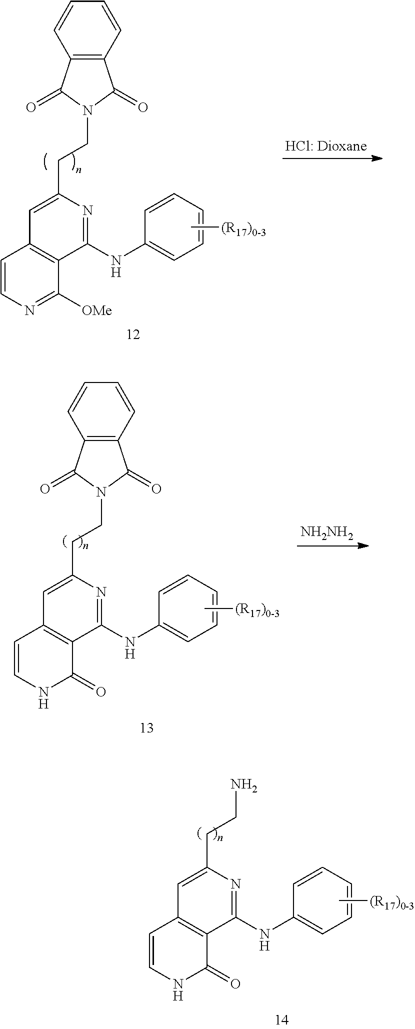

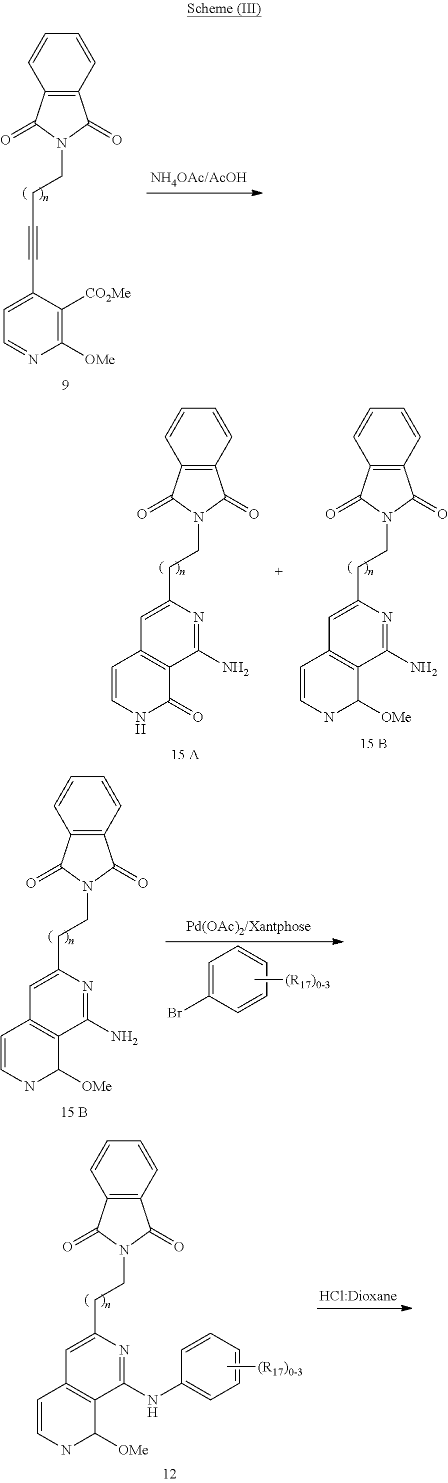

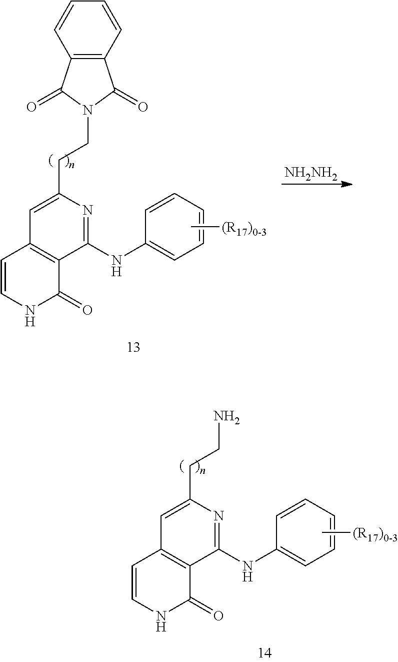

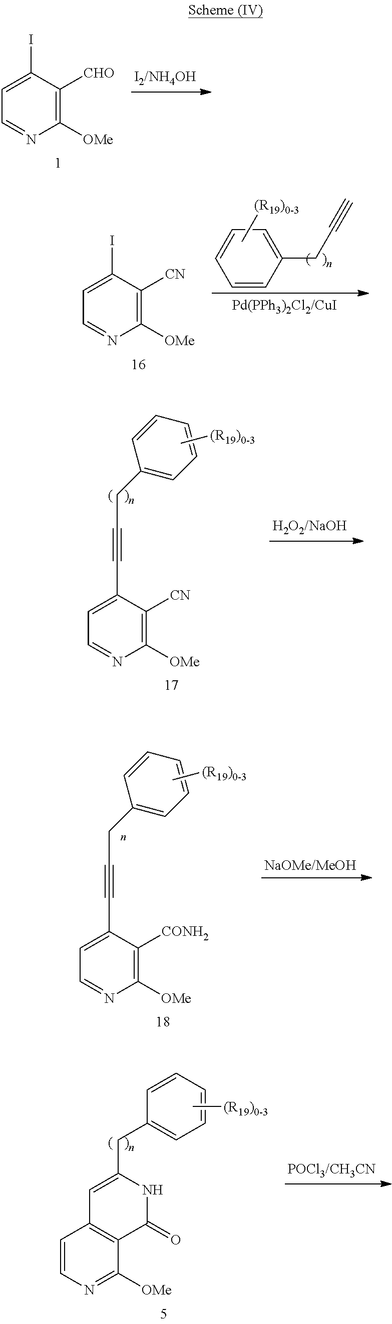

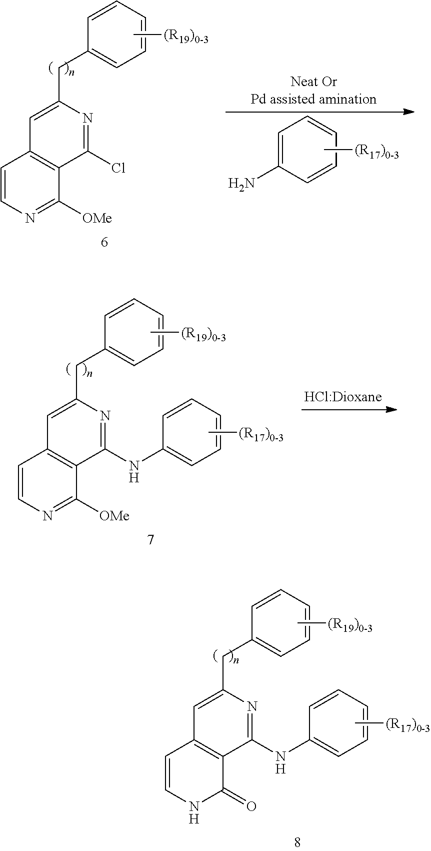

The synthesis of certain intermediates used in the synthesis of compounds of Formula (I) is illustrated in scheme (V) and scheme (VI).

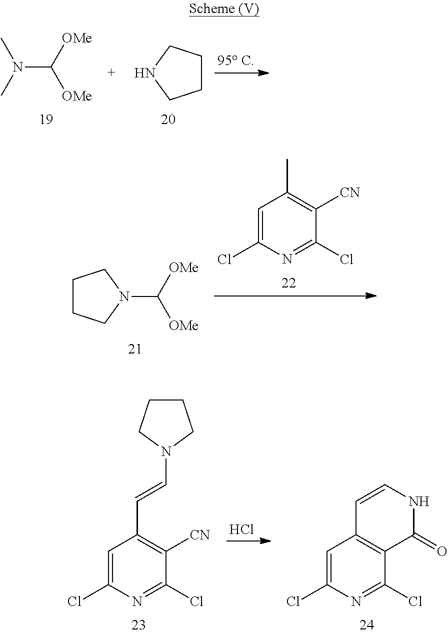

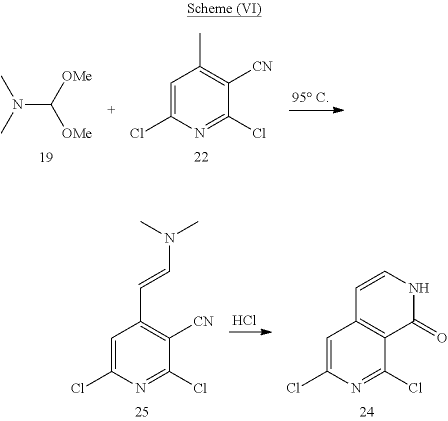

The synthesis of certain compounds of Formula (I) using the 6,8-dichloro-2,7-naphthyridin-1(2H)-one (24) intermediate compound is illustrated in scheme (VII) and scheme (VIII).

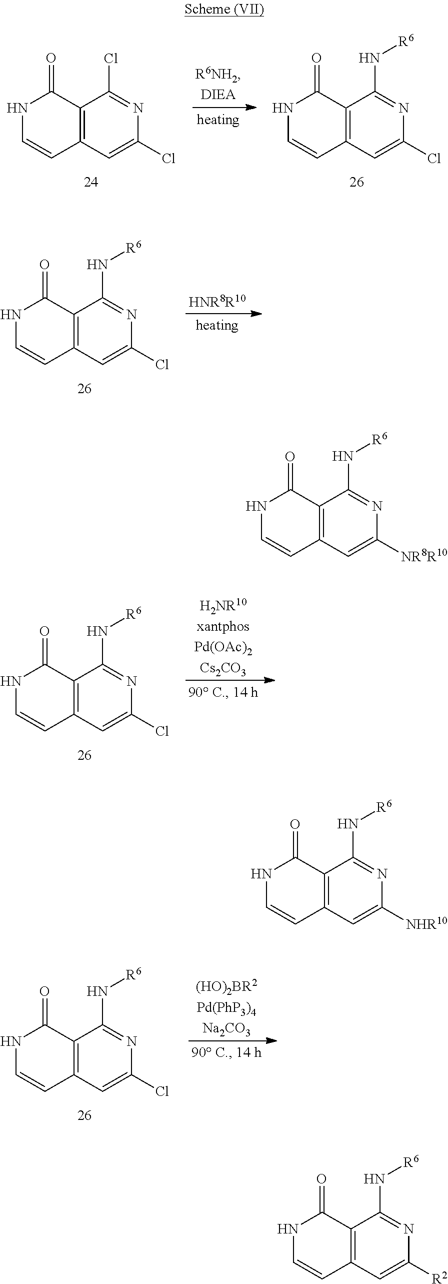

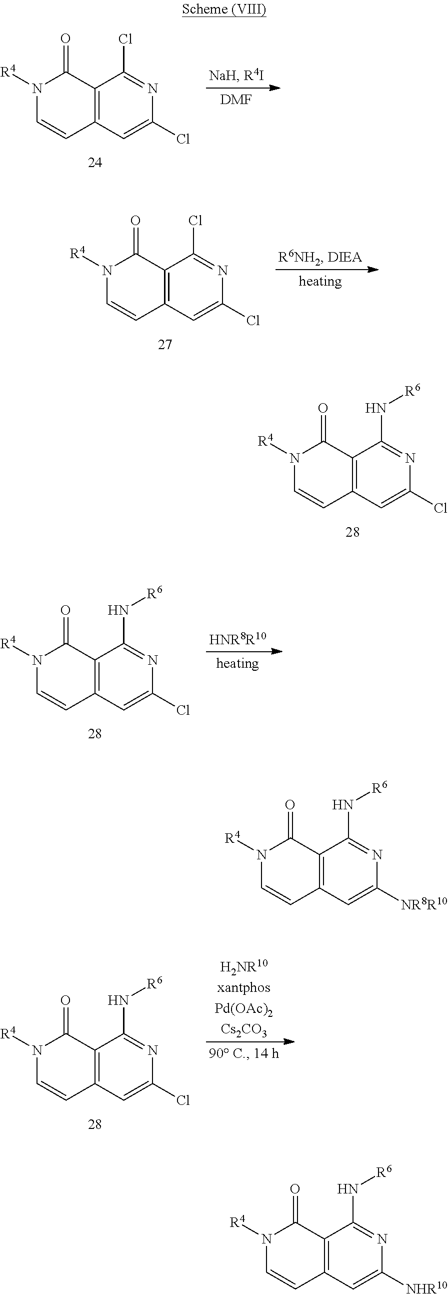

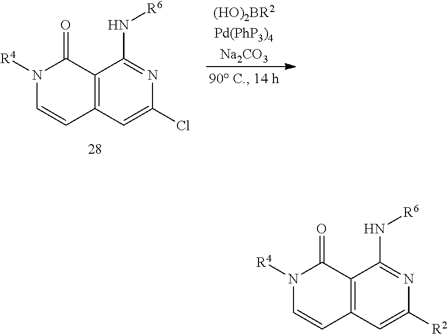

The R², R⁴, R⁶, R⁸, R¹⁰, R¹⁷, and R¹⁹⁶ of Schemes (I) to (IV) and (VII) to (VIII) are as defined herein.

### EXAMPLES

The present invention is further exemplified, but not limited, by the following examples that illustrate the preparation of compounds of Formula (I) according to the invention.

### Example 1

### Preparation of 8-(4-(1-(3-methoxy-2,2-dimethylpropanoyl)piperidin-4-yl)-3-methylphenylamino)-6-(6-methoxypyrazin-2-yl)-2,7-naphthyridin-1(2H)-one

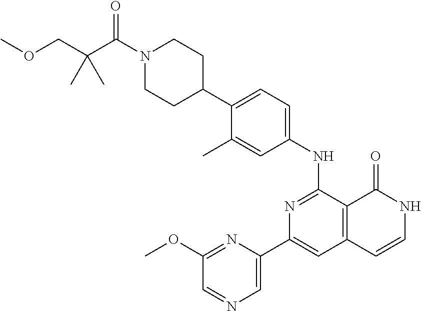

### Example 1a

### tert-butyl 4-(4-(3-chloro-8-oxo-7,8-dihydro-2,7-naphthyridin-1-ylamino)-2-methylphenyl)piperidine-1-carboxylate

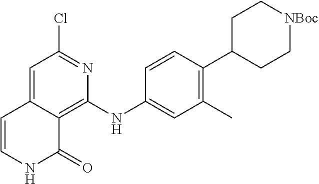

A suspension of 6,8-dichloro-2,7-naphthyridin-1(2H)-one (150 mg, 0.70 mmol) and tert-butyl 4-(4-amino-2-methylphenyl)piperidine-1-carboxylate (203 mg, 0.70 mmol) in 1 mL 2-propanol was irradiated by microwave at 170° C. for 45 minutes. LC/MS showed quantitative conversion and the crude product was filtered and washed with 10% ethyl acetate/hexane. The crude brown solid was used for the next reaction step without any further purification. MS m/z 469.19 (M+1).

### Example 1b

### tert-butyl 4-(4-(3-(6-methoxypyrazin-2-yl)-8-oxo-7,8-dihydro-2,7-naphthyridin-1-ylamino)-2-methylphenyl)piperidine-1-carboxylate

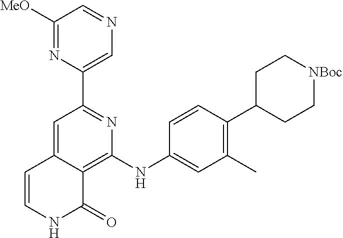

A mixture of tert-butyl 4-(4-(3-chloro-8-oxo-7,8-dihydro-2,7-naphthyridin-1-ylamino)-2-methylphenyl)piperidine-1-carboxylate (100 mg, 0.21 mmol), 2-methoxy-6-(tributylstannyl)pyrazine (92.3 mg, 0.23 mmol), PdCl₂dppf (19 mg, 0.021 mmol) in dimethyl formamide (DMF) (1.5 mL) was purged with N₂ and irradiated by microwave at 135° C. for 1 hour. The DMF was removed and the compound was purified by silica gel column chromatography using 15% ethyl acetate/hexane as eluent. MS m/z 543.19 (M+1).

Deprotection of the Boc group was performed by treating the above compound with 6 N HCl (0.2 mL) in 1 mL of dichloromethane (DCM). The mixture was stirred for another 14 hours at 60° C. The desired compound was precipitated out, filtered and washed with 20% ethyl acetate/hexane. ¹H NMR (400 MHz, DMSO-d6) δ 11.90 (s, 1H), 11.89 (brs, 1H), 9.04 (s, 1H), 8.42 (s, 1H), 7.82 (s, 1H), 7.73 (dd, 1H), 7.62 (s, 1H), 7.42 (t, 1H), 7.19 (d, 1H), 6.64 (d, 1H), 4.06 (s, 3H), 3.59-3.58 (m, 2H), 3.30-3.27 (m, 2H), 2.82-2.76 (m, 1H), 2.39 (s, 3H) 1.80 (brm, 4H); MS m/z 443.14 (M+1).

### Example 1c

### 8-(4-(1-(3-methoxy-2,2-dimethylpropanoyl)piperidin-4-yl)-3-methylphenylamino)-6-(6-methoxypyrazin-2-yl)-2,7-naphthyridin-1(2H)-one

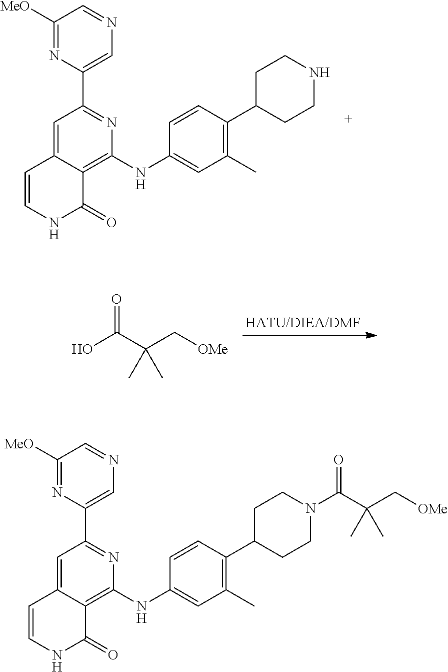

DIEA (10 uL) was added to a suspension of 3-methoxy-2,2-dimethylpropanoic acid (6.5 mg, 0.048 mM) and HATU (18 mg, 0.048 mM) in DMF (0.5 mL). 6-(6-methoxypyrazin-2-yl)-8-(3-methyl-4-(piperidin-4-yl)phenylamino)-2,7-naphthyridin-1(2H)-one (18 mg, 0.04 mM) was added to the above solution and the reaction mixture was allowed to stir for 4 hours. The DMF was removed and the residue dissolved in DMSO. The compound was purified by preparative HPLC to afford the title compound as a TFA salt. ¹H NMR (400 MHz, DMSO-d6) δ 11.94 (s, 1H), 11.88 (brs, 1H), 9.07 (s, 1H), 8.40 (s, 1H), 7.81 (s, 1H), 7.74 (dd, 1H), 7.65 (s, 1H), 7.46 (t, 1H), 7.22 (d, 1H), 6.67 (d, 1H), 4.07 (s, 3H), 3.59-3.58 (m, 2H), 3.30-3.27 (m, 2H), 3.14-3.06 (m, 2H), 2.82-2.76 (m, 1H), 2.54 (s, 2H), 2.39 (s, 3H) 2.14 (m, 2H), 1.22 (s, 6H); MS m/z 557.1 (M+1).

### Example 2

### Preparation of 8-(4-((1r,4r)-4-morpholinocyclohexyl)phenylamino)-6-(pyrimidin-5-yl)-2,7-naphthyridin-1(2H)-one

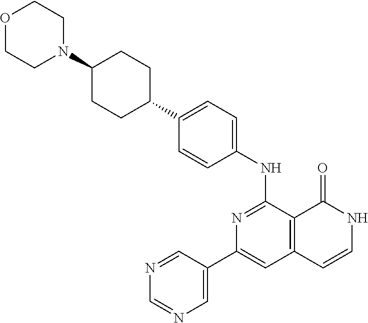

### Example 2a

### 6-chloro-8-(4-((1r,4r)-4-morpholinocyclohexyl)phenylamino)-2,7-naphthyridin-1(2H)-one

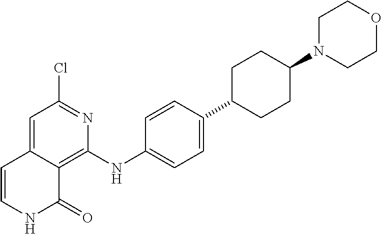

A suspension of 6,8-dichloro-2,7-naphthyridin-1(2H)-one (150 mg, 0.70 mmol) and 4-((1r,4r)-4-morpholinocyclohexyl)aniline (182 mg, 0.70 mmol) in 1 mL of 2-propanol was irradiated by microwave at 170° C. for 45 minutes. LC/MS showed quantitative conversion and the crude product was filtered and washed with 10% ethyl acetate/hexane. The crude yellow solid was used in next reaction step without any further purification. MS m/z 439.20 (M+1).

### Example 2b

### 8-(4-((1r,4r)-4-morpholinocyclohexyl)phenylamino)-6-(pyrimidin-5-yl)-2,7-naphthyridin-1(2H)-one

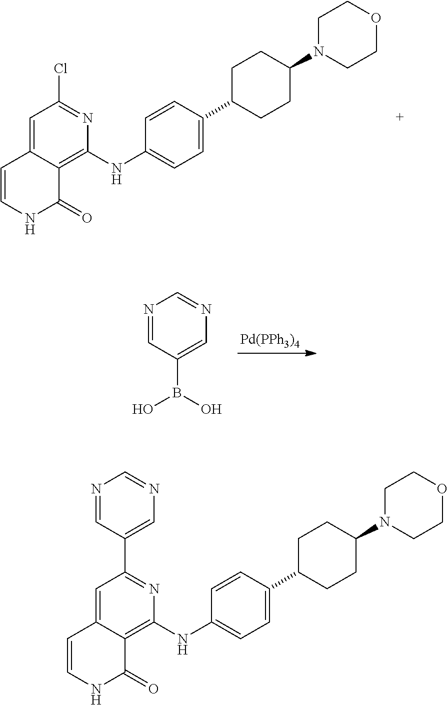

A mixture of 6-chloro-8-(4-((1r,4r)-4-morpholinocyclohexyl)phenylamino)-2,7-naphthyridin-1(2H)-one (20 mg, 0.045 mmol), pyrimidin-5-ylboronic acid (5.6 mg, 0.0.045 mmol), Pd(PPh₃)₄ (4.6 mg, 0.004 mmol) and Na₂CO₃ (24 mg, 0.23 mol) in a mixed solvent of dioxane (1.5 mL) and H₂O (0.5 mL) was purged with N₂, heated at 90° C. for 16 hours, evaporated to result in a residue which was diluted with DMSO, acidified with TFA, and subject to HPLC purification to afford the TFA salt of the title compound. ¹H NMR (400 MHz, DMSO-d6) δ 11.97 (s, 1H), 11.94 (brs, 1H), 9.45 (s, 2H), 9.28 (s, 1H), 7.76 (d, 2H), 7.64 (s, 1H), 7.47 (t, 1H), 7.27 (d, 2H), 6.55 (d, 1H), 3.96-3.95 (m, 4H), 3.40 (m, 2H), 3.21-3.10 (m, 2H), 2.27 (d, 2H), 1.98 (d, 2H), 1.98 (d, 2H), 1.71-1.63 (m, 2H), 1.57-1.51 (m, 2H); MS m/z 483.3 (M+1).

### Example 3

### Preparation of 3-(4-(4-(3-(6-methoxypyrazin-2-yl)-8-oxo-7,8-dihydro-2,7-naphthyridin-1-ylamino)-2-methylphenyl)piperidin-1-yl)propanenitrile

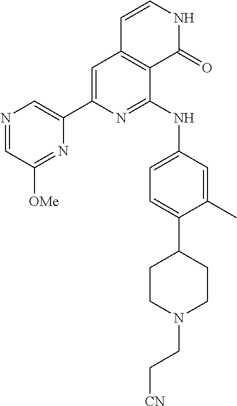

### Example 3a

tert-butyl 4-(4-(3-chloro-8-oxo-7-((2-(trimethylsilyl)ethoxy)methyl)-7,8-dihydro-2,7-naphthyridin-1-ylamino)-2-methylphenyl)piperidine-1-carboxylate

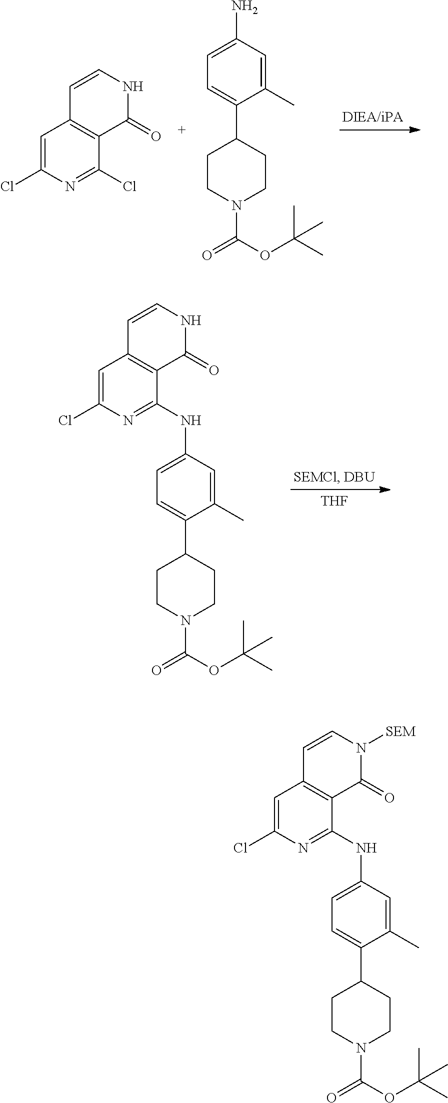

To a solution of 6,8-dichloro-2,7-naphthyridin-1(2H)-one (0.67 g, 3.13 mmol) and DIEA (817 μL, 4.69 mmol) in 10 mL of isopropanol was added tert-butyl 4-(4-amino-2-methylphenyl)piperidine-1-carboxylate (1.00 g 3.45 mmol). The mixture was irradiated at 150° C. for 1 hour and then 2 mL of water was added. The mixture was then stirred at ambient temperature overnight. The precipitate was collected by filtration to provide tert-butyl 4-(4-(3-chloro-8-oxo-7,8-dihydro-2,7-naphthyridin-1-ylamino)-2-methylphenyl)piperidine-1-carboxylate.

To a solution of tert-butyl 4-(4-(3-chloro-8-oxo-7,8-dihydro-2,7-naphthyridin-1-ylamino)-2-methylphenyl)piperidine-1-carboxylate (1.00 g, 2.12 mmol) in 20 mL of anhydrous THF was added DBU (637 μL, 4.26 mmol) and trimethylsilylethoxymethychloride (564 μL, 3.20 mmol). The mixture was stirred at ambient temperature overnight and then 100 mL of ethyl acetate (EtOAc) was added. The organic layer was washed with water and brine solution, dried over MgSO₄, filtered and concentrated. The residue was purified by column chromatography on a silica gel (EtOAc:Hexane=6:1) to provide tert-butyl 4-(4-(3-chloro-8-oxo-7-((2-(trimethylsilyl)ethoxy)methyl)-7,8-dihydro-2,7-naphthyridin-1-ylamino)-2-methylphenyl)piperidine-1-carboxylate. ¹H NMR (DMSO-d₆) δ 1.50 (s, 9H), 1.60 (m, 2H), 1.76 (d, J=12.8 Hz, 2H), 2.37 (s, 3H), 2.93 (m, 3H), 4.23 (d, J=13.2 Hz, 2H), 6.42 (d, J=7.2 Hz, 1H), 6.75 (s, 1H), 7.15 (d, J=8.4 Hz, 1H), 7.29 (d, J=7.2 Hz, 1H), 7.49 (d, J=2.0 Hz, 1H), 7.65 (dd, J=8.4 and 2.0 Hz, 1H), 11.77 (s, 1H); ESI-MS m/z 600.2 (MH⁺).

### Example 3b

### tert-butyl 4-(4-(3-(6-methoxypyrazin-2-yl)-8-oxo-7-((2-(trimethylsilyl)ethoxy)methyl)-7,8-dihydro-2,7-naphthyridin-1-ylamino)-2-methylphenyl)piperidine-1-carboxylate

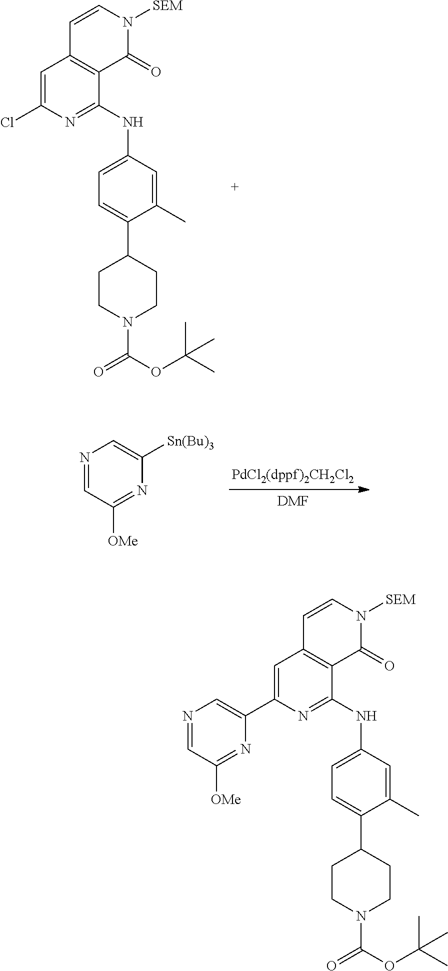

To a solution of tert-butyl 4-(4-(3-chloro-8-oxo-7((2-(trimethylsilyl)ethoxy)methyl)-7,8-dihydro-2,7-naphthyridin-1-ylamino)-2-methylphenyl)piperidine-1-carboxylate (1.00 g, 1.67 mmol) in 20 mL of anhydrous DMF was added 2-methoxy-6-(tributylstannyl)pyrazine (799 mg, 2.00 mmol) and PdCl₂(dppf)₂CH₂Cl₂ (136 mg, 0.16 mmol). After being irradiated at 135° C. for 1 hour, the reaction mixture was added to 100 mL of EtOAc and 100 mL of diethylether, and then washed with 100 mL of brine solution three times. The organic layer was dried over MgSO₄, filtered and concentrated. The residue was purified by column chromatography on a silica gel (EtOAc:Hexane=1:3) to provide tert-butyl 4-(4-(3-(6-methoxypyrazin-2-yl)-8-oxo-7-((2-(trimethylsilyl)ethoxy)methyl)-7,8-dihydro-2,7-naphthyridin-1-ylamino)-2-methylphenyl)piperidine-1-carboxylate. ESI-MS m/z 673.2 (MH⁺).

### Example 3c

### 3-(4-(4-(3-(6-methoxypyrazin-2-yl)-8-oxo-7,8-dihydro-2,7-naphthyridin-1-ylamino)-2-methylphenyl)piperidin-1-yl)propanenitrile

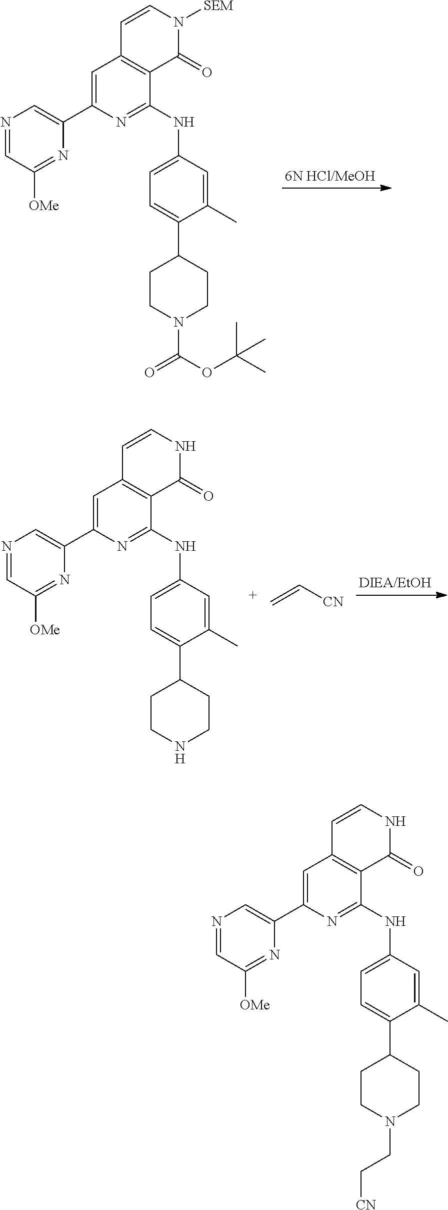

To a solution of tert-butyl 4-(4-(3-(6-methoxypyrazin-2-yl)-8-oxo-7-((2-(trimethylsilyl)ethoxy)methyl)-7,8-dihydro-2,7-naphthyridin-1-ylamino)-2-methylphenyl)piperidine-1-carboxylate (640 mg, 0.91 mmol) in 10 mL of methanol (MeOH) was added 10 mL of 6N HCl. The mixture was stirred at 60° C. overnight and concentrated by rotary evaporation. 20 mL of MeOH was added to the residue and the precipitate was collected by filtration to provide 6-(6-methoxypyrazin-2-yl)-8-(3-methyl-4-(piperidin-4-yl)phenylamino)-2,7-naphthyridin-1(2H)-one. To a solution of 6-(6-methoxypyrazin-2-yl)-8-(3-methyl-4-(piperidin-4-yl)phenylamino)-2,7-naphthyridin-1(2H)-one (20 mg, 0.045 mmol) in 2 mL of EtOH was added DIEA (19.6 μL, 0.113 mmol) and acrylonitrile (6.0 μL, 0.09 mmol). After being stirred at 40° C. overnight, the precipitate was collected by filtration to provide 3-(4-(4-(3-(6-methoxypyrazin-2-yl)-8-oxo-7,8-dihydro-2,7-naphthyridin-1-ylamino)-2-methylphenyl)piperidin-1-yl)propanenitrile (21 mg, 93.9% yield) as a yellowish powder. ¹H NMR (DMSO-d₆) δ 1.98 (s, 3H), 2.40 (s, 3H), 3.03 (bs, 1H), 3.17 (m, 4H), 3.48 (d, J=4.8 Hz, 2H), 3.60 (d, J=10.8 Hz, 2H), 4.09 (s, 3H), 6.68 (dd, J=7.2 and 1.2 Hz, 1H), 7.20 (d, J=8.4 Hz, 1H), 7.48 (t, J=6.4 Hz, 1H), 7.70 (s, 1H), 7.78 (d, J=8.4 Hz, 1H), 7.83 (s, 1H), 8.41 (s, 1H), 9.07 (s, 1H), 10.42 (bs, 1H), 11.92 (d, J=5.6 Hz, 1H), 11.98 (s, 1H); ESI-MS m/z 496.2 (MH⁺).

### Example 4

### Preparation of 6-(2-aminopyrimidin-5-yl)-8-(1-hydroxypropan-2-ylamino)-2,7-naphthyridin-1(2H)-one

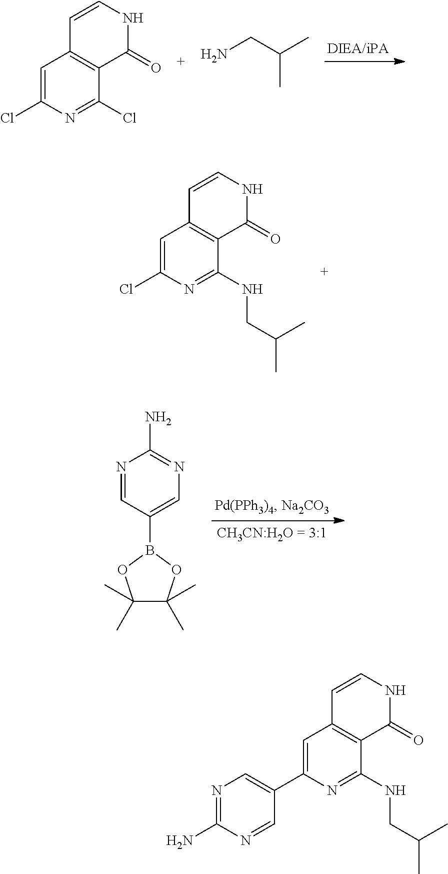

To a solution 6,8-dichloro-2,7-naphthyridin-1(2H)-one (400 mg, 1.87 mmol) in 20 mL of isopropanol was added DIEA (488 μL, 2.80 mmol) and 2-aminopropanol (223 μL, 2.80 mmol). The mixture was irradiated at 150° C. for 1 hour and treated with 10 mL of H₂0. The precipitate was collected by filtration to provide 6-chloro-8-(1-hydroxypropan-2-ylamino)-2,7-naphthyridin-1(2H)-one. To a solution of 6-chloro-8-(1-hydroxypropan-2-ylamino)-2,7-naphthyridin-1(2H)-one (20 mg, 0.080 mmol) in 2 mL of CH₃CN was added Pd(PPh3)4 (7.0 mg, 10% mol), Na₂CO₃ (16 mg, 0.199 mmol) and 0.7 mL of H₂0. The mixture was irradiated at 120° C. for 0.5 hour and purified on a reverse phase C-18 preparative HPLC eluting with acetonitrile (0.05% TFA)/water (0.0375% TFA). After lyophilization, 6-(2-aminopyrimidin-5-yl)-8-(1-hydroxypropan-2-ylamino)-2,7-naphthyridin-1(2H)-one was obtained. ¹H NMR (DMSO-d₆) δ 0.98 (d, J=6.4 Hz, 6H), 1.97 (m, 1H), 3.39 (t, J=6.0 Hz, 2H), 3.35 (d, J=6.0 Hz, 1H), 7.09 (s, 1H), 7.17 (bs, 1H), 7.30 (t, J=6.4 Hz, 1H), 8.95 (s, 2H), 9.55 (bs, 1H), 11.41 (bs, 1H); ESI-MS m/z 311.

### Example 5

### Preparation of 8-(isopropylamino)-6-(4-methylpyridin-2-ylamino)-2,7-naphthyridin-1(2H)-one

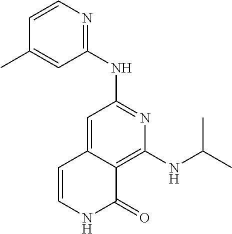

### Example 5a

### 6-chloro-8-(isopropylamino)-2,7-naphthyridin-1(2H)-one

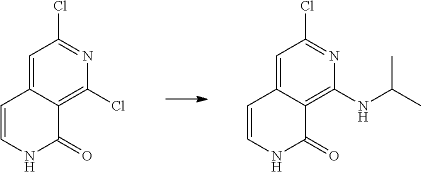

6,8-dichloro-2,7-naphthyridin-1(2H)-one (1.0752 gram, 5 mmol), 2-propanamine (426 μL, 1.15 equiv) and DIEA (875 μL, 1.0 equiv) were dissolved in 2-propanol (3.0 mL). The mixture was heated with microwave at 150° C. for 30 minutes. The mixture was filtered after cool down. The solid was collected, washed with 2-propanol several times and dried to give 6-chloro-8-(isopropylamino)-2,7-naphthyridin-1(2H)-one. ¹H NMR (400 MHz, DMSO-d6): δ 11.58 (s, 1H), 9.48 (s, 1H), 7.33 (d, J=7.2 Hz, 1H), 6.63 (s, 1H), 6.34 (d, J=7.2 Hz, 1H), 4.17 (hep, J=6.4 Hz, 1H), 1.21 (d, J=6.4 Hz, 6H); ¹³C NMR (100 MHz, DMSO-d6): δ 163.05, 157.80, 151.53, 148.47, 134.28, 104.62, 103.51, 103.42, 41.64, 22.29; ESI-MS (m/z) 238.07 (MH⁺).

### Example 5b

### 8-(isopropylamino)-6-(4-methylpyridin-2-ylamino)-2,7-naphthyridin-1(2H)-one

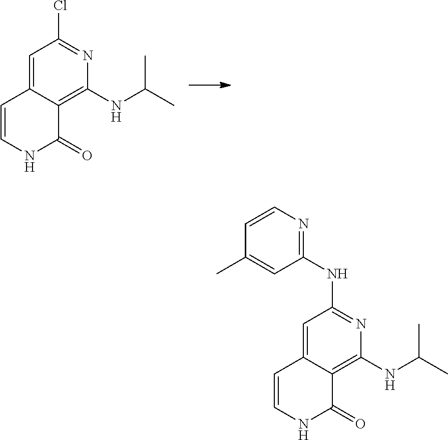

1.0 mL tert-Butanol was added to the mixture of 6-chloro-8-(isopropylamino)-2,7-naphthyridin-1(2H)-one (19.0 mg, 0.08 mmol), 4-methylpyridin-2-amine (17.3 mg, 0.16 mmol), Pd₂(DBA)₃ (3.6 mg, 5%), Xantphos (6.8 mg, 20%), Cs₂CO₃ (78.2 mg, 300%). The mixture was heated at 150° C. for 3 hours using an oil bath. The mixture was filtered and purified by HPLC to obtain 8-(isopropylamino)-6-(4-methylpyridin-2-ylamino)-2,7-naphthyridin-1(2H)-one. ¹H NMR (400 MHz, MeOD): δ 8.21 (d, J=6.0 Hz, 1H), 7.31 (d, J=6.8 Hz, 1H), 7.12 (d, J=6.0 Hz, 1H), 7.07 (s, 1H), 6.39 (d, J=6.8 Hz, 1H), 6.18 (s, 1H), 4.16 (hep, J=6.4 Hz, 1H), 2.50 (s, 3H), 1.46 (d, J=6.4 Hz, 6H); ESI-MS (m/z) 310.16 (MH⁺).

### Example 6

### Preparation of 8-(isopropylamino)-2-methyl-6-(4-methylpyridin-2-ylamino)-2,7-naphthyridin-1(2H)-one

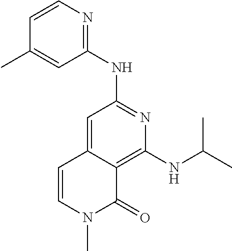

### Example 6a

### 6-chloro-8-(isopropylamino)-2-methyl-2,7-naphthyridin-1(2H)-one

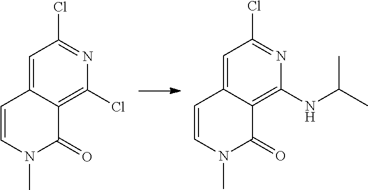

6,8-dichloro-2-methyl-2,7-naphthyridin-1(2H)-one (183.3 mg, 0.8 mmol), 2-propanamine (78.4 μL, 1.15 equiv) and DIEA (138.7 μL, 1.0 equiv) were dissolved in 2-propanol (1.5 mL). The mixture was heated with microwave at 150° C. for 30 minutes and filtered after cooling to room temperature. The solid was collected, washed with 2-propanol several times and dried to give 6-chloro-8-(isopropylamino)-2-methyl-2,7-naphthyridin-1(2H)-one. ¹H NMR (400 MHz, MeOD): δ 10.38 (s, 1H), 8.47 (d, J=7.2 Hz, 1H), 7.43 (s, 1H), 7.21 (d, J=7.2 Hz, 1H), 4.25 (s, 3H), 3.91 (hep, J=6.4 Hz, 1H), 2.01 (d, J=6.4 Hz, 6H); ESI-MS (m/z) 252.71 (MH⁺).

### Example 6b

### 8-(isopropylamino)-2-methyl-6-(4-methylpyridin-2-ylamino)-2,7-naphthyridin-1(2H)-one

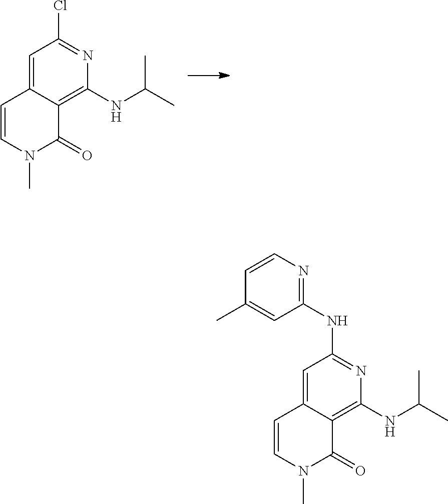

1.0 mL tert-Butanol was added to a mixture of 6-chloro-8-(isopropylamino)-2-methyl-2,7-naphthyridin-1(2H)-one (20.1 mg, 0.08 mmol), 4-methylpyridin-2-amine (17.3 mg, 0.16 mmol), Pd₂(DBA)₃ (3.6 mg, 5%), Xantphos (6.8 mg, 20%) and Cs₂CO₃ (78.2 mg, 300%). The mixture was heated at 150° C. for 3 hours using an oil bath. The mixture was filtered and purified by HPLC to obtain 8-(isopropylamino)-2-methyl-6-(4-methylpyridin-2-ylamino)-2,7-naphthyridin-1(2H)-one. ¹H NMR (400 MHz, MeOD): δ 8.17 (d, J=6.0 Hz, 1H), 7.48 (d, J=7.2 Hz, 1H), 7.10 (d, J=6.0 Hz, 1H), 7.05 (s, 1H), 6.36 (d, J=7.2 Hz, 1H), 6.13 (s, 1H), 4.09 (hep, J=6.4 Hz, 1H), 3.48 (s, 3H), 2.48 (s, 3H), 1.46 (d, J=6.4 Hz, 6H); ¹³C NMR (100 MHz, MeOD): δ 164.34, 157.57, 154.40, 153.25, 149.41, 144.37, 138.80, 119.48, 114.56, 106.18, 100.82, 92.32, 44.29, 36.98, 22.85, 21.64; ESI-MS (m/z) 324.39 (MH⁺).

### Example 7

### Preparation of 8-(tert-butylamino)-2-(2-hydroxyethyl)-6-(pyrazin-2-ylamino)-2,7-naphthyridin-1(2H)-one

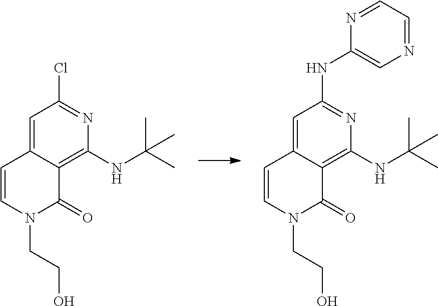

1.0 mL tert-Butanol was added to a mixture of 8-(tert-butylamino)-6-chloro-2-(2-hydroxyethyl)-2,7-naphthyridin-1(2H)-one (23.7 mg, 0.08 mmol), 2-aminopyrazine (15.2 mg, 0.16 mmol), Pd₂(DBA)₃ (3.6 mg, 5%), Xantphos (6.8 mg, 20%) and Cs₂CO₃ (78.2 mg, 300%). The mixture was heated at 150° C. for 3 hours using an oil bath. The mixture was then filtered and purified by HPLC to give 8-(tert-butylamino)-2-(2-hydroxyethyl)-6-(pyrazin-2-ylamino)-2,7-naphthyridin-1(2H)-one. ¹H NMR (400 MHz, MeOD): δ 8.51 (s, 1H), 8.37 (d, J=3.2 Hz, 1H), 8.34 (d, J=3.2 Hz, 1H), 7.68 (d, J=7.2 Hz, 1H), 6.52 (d, J=7.2 Hz, 1H), 6.32 (s, 1H), 4.11 (t, J=5.2 Hz, 2H), 3.84 (t, J=5.2 Hz, 2H), 1.71 (s, 9H); ESI-MS m/z 355.18 (MH+).

### Example 8 (Compound No. 600)

### Preparation of 8-(tert-butylamino)-6-(4-((3-hydroxyazetidin-1-yl)methyl)pyridin-2-ylamino)-2-(2-hydroxyethyl)-2,7-naphthyridin-1(2H)-one

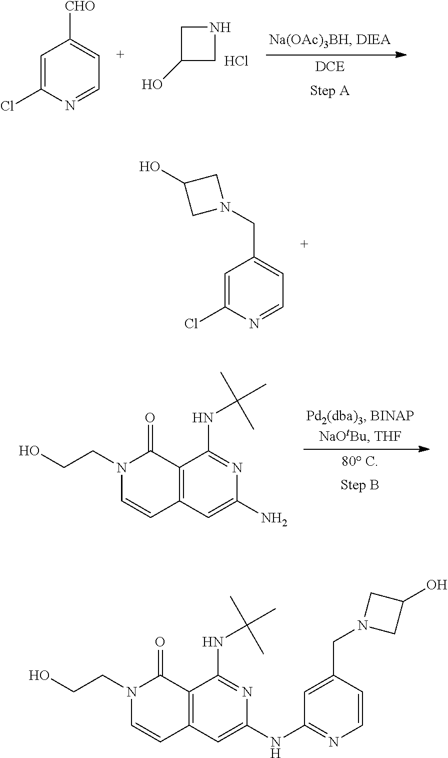

Step A: A mixture of 2-chloroisonicotinaldehyde (56.0 mg, 0.4 mmol), azetidin-3-ol hydrochloride (38.0 mg, 0.4 mmol), DIEA (0.2 mL, 1.2 mmol) and Na(OAc)₃BH (101.3 mg, 0.48 mmol) in 2.0 mL of DCE was stirred at room temperature for 1 hour. The reaction mixture was partitioned between DCM and NH₄Cl, and the collected organic extracts were dried (Na₂SO₄), concentrated in vacuo, followed by chromatography (EtOAc/hexanes: 0-50%) to afford 1-((2-chloropyridin-4-yl)methyl)azetidin-3-ol. ESI-MS m/z 199.1 (MH⁺).

Step B: A mixture of 1-((2-chloropyridin-4-yl)methyl)azetidin-3-ol (10.0 mg, 0.05 mmol), 6-amino-8-(tert-butylamino)-2-(2-hydroxyethyl)-2,7-naphthyridin-1(2H)-one (14.0 mg, 0.05 mmol), Pd₂(dba)₃ (5.0 mg, 0.005 mmol), BINAP (3.1 mg, 0.005 mmol) and NaO^{t}Bu (10.0 mg, 0.11 mmol) in 0.5 mL of THF was degassed and purged with N₂, then heated at 85° C. for 45 minutes. The reaction mixture was purified on a preparation HPLC to afford 8-(tert-butylamino)-6-(44(3-hydroxyazetidin-1-yl)methyl)pyridin-2-ylamino)-2-(2-hydroxyethyl)-2,7-naphthyridin-1(2H)-one. ESI-MS m/z 439.2 (MH⁺).

### Example 9 (Compound No. 602)

### Preparation of 8-(tert-butylamino)-2-(2-hydroxyethyl)-6-(4-(2,2,2-trifluoro-1-hydroxyethyl)pyridin-2-ylamino)-2,7-naphthyridin-1(2H)-one

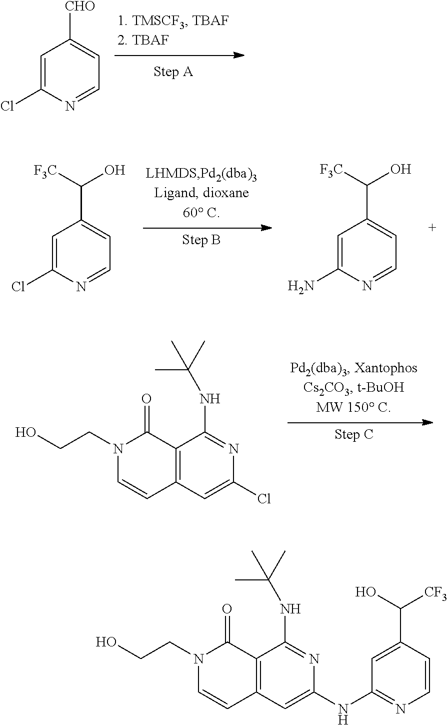

Step A: Under nitrogen, to a solution of TMSCF₃ (0.5N in THF, 5.4 mL, 2.7 mmol) in 10 mL of THF at 0° C. was added 2-chloroisonicotinaldehyde (282.0 mg, 2.0 mmol) and 0.1 mL of TBAF sequentially, and the reaction was stirred at 0° C. until the starting material was completely consumed. Another 0.4 mL of TBAF was added, and the reaction was warmed to room temperature and stirred for 1 hour. The reaction was quenched with NH₄Cl, and extracted with EtOAc. The combined organic extracts were dried (Na₂SO₄), concentrated in vacuo, followed by chromatography (EtOAc/hexanes: 0-50%) to afford 1-(2-chloropyridin-4-yl)-2,2,2-trifluoroethanol. ESI-MS m/z 212.0 (MH⁺).

Step B: A mixture of 1-(2-chloropyridin-4-yl)-2,2,2-trifluoroethanol (80.0 mg, 0.38 mmol), LHMDS (1.0 N in THF, 1.1 mL, 1.1 mmol), Pd₂(dba)₃ (17.4 mg, 0.02 mmol), biphenyl-2-yl(cycloheptyl)(cyclohexyl)phosphine (13.3 mg, 0.04 mmol) in 1.0 mL of 1,4-dioxane was degassed and purged with N₂, then heated at 60° C. overnight. The reaction mixture was partitioned between EtOAc and brine, the combined organic extracts were dried (Na₂SO₄), concentrated in vacuo, followed by chromatography (MeOH/DCM: 0-10%) to afford 1-(2-aminopyridin-4-yl)-2,2,2-trifluoroethanol. ESI-MS m/z 193.1 (MH⁺).

Step C: A mixture of 1-(2-aminopyridin-4-yl)-2,2,2-trifluoroethanol (51.1 mg, 0.27 mmol), 8-(tert-butylamino)-6-chloro-2-(2-hydroxyethyl)-2,7-naphthyridin-1(2H)-one (78.5 mg, 0.27 mmol), Pd₂(dba)₃ (24.0 mg, 0.027 mmol), Xantophos (15.4 mg, 0.027 mmol) and Cs₂CO₃ (259.0 mg, 0.80 mmol) in 2.0 mL of t-BuOH was degassed and purged with nitrogen, the mixture was heated at 150° C. in microwave for 30 min. The reaction mixture was purified on a preparation HPLC to afford 8-(tert-butylamino)-2-(2-hydroxyethyl)-6-(4-(2,2,2-trifluoro-1-hydroxyethyl)pyridin-2-ylamino)-2,7-naphthyridin-1(2H)-one. ESI-MS m/z 452.2 (MH⁺).

### Example 10 (Compound No. 606)

### Preparation of 8-(tert-butylamino)-2-(2-hydroxyethyl)-6-(5-methoxypyridin-2-ylamino)-2,7-naphthyridin-1(2H)-one

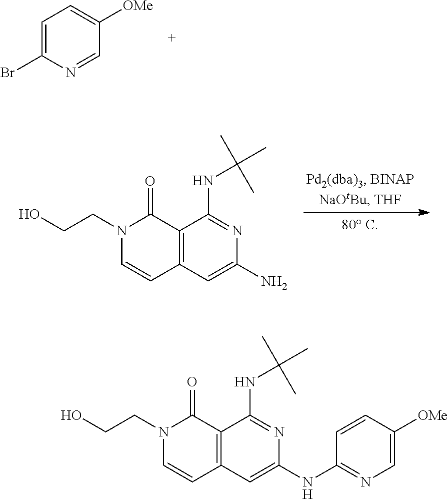

A mixture of 2-bromo-5-methoxypyridine (11.2 mg, 0.0 g mmol), 6-amino-8-(tert-butylamino)-2-(2-hydroxyethyl)-2,7-naphthyridin-1(2H)-one (16.0 mg, 0.06 mmol), Pd₂(dba)₃ (5.4 mg, 0.006 mmol), BINAP (3.6 mg, 0.006 mmol) and NaO^{t}Bu (12.2 mg, 0.13 mmol) in 0.5 mL of THF was degassed and purged with N₂, then heated at 85° C. for 45 minutes. The reaction mixture was purified on a preparation HPLC to afford 8-(tert-butylamino)-2-(2-hydroxyethyl)-6-(5-methoxypyridin-2-ylamino)-2,7-naphthyridin-1(2H)-one. ESI-MS m/z 384.3 (MH⁺).

### Example 11 (Compound No. 607)

### Preparation of 8-(tert-butylamino)-2-(2-hydroxyethyl)-6-(6-(hydroxymethyl)pyrimidin-4-ylamino)-2,7-naphthyridin-1(2H)-one

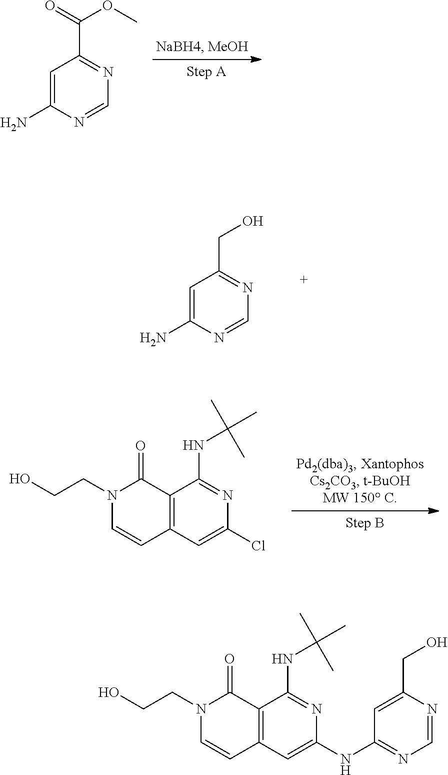

Step A: To a solution of methyl 6-aminopyrimidine-4-carboxylate (30.0 mg, 0.2 mmol) in 0.5 mL of MeOH was added NaBH₄(38.0 mg, 1.0 mmol), and the reaction was heated to reflux for 3 hours. The reaction mixture was concentrated, and the crude was used in step B directly.

Step B: A mixture of the crude from step A, 8-(tert-butylamino)-6-chloro-2-(2-hydroxyethyl)-2,7-naphthyridin-1(2H)-one (59.0 mg, 0.2 mmol), Pd₂(dba)₃ (24.0 mg, 0.02 mmol), Xantophos (15.4 mg, 0.02 mmol) and Cs₂CO₃ (259.0 mg, 0.80 mmol) in 2.0 mL of t-BuOH was degassed and purged with nitrogen, the mixture was heated at 150° C. in microwave for 30 minutes. The reaction mixture was purified on a preparation HPLC to afford 8-(tert-butylamino)-2-(2-hydroxyethyl)-6-(6-(hydroxymethyl)pyrimidin-4-ylamino)-2,7-naphthyridin-1(2H)-one. ESI-MS m/z 385.2 (MH⁺).

### Example 12 (Compound No. 609)

### Preparation of 8-(tert-butylamino)-2-(2-hydroxyethyl)-6-(4-(3-hydroxyoxetan-3-yl)pyridin-2-ylamino)-2,7-naphthyridin-1(2H)-one

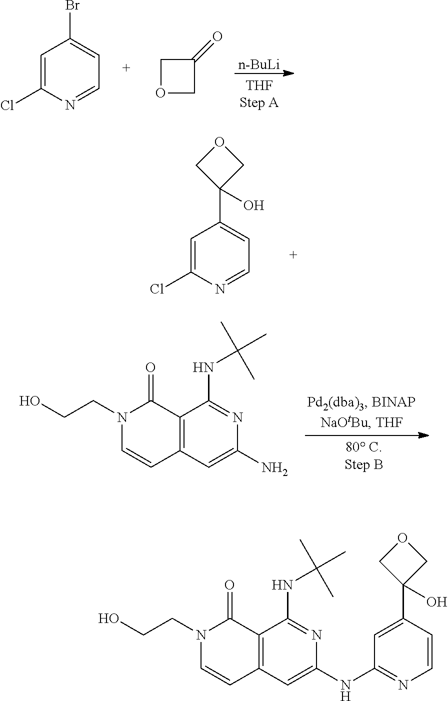

Step A: Under nitrogen, to a solution of n-BuLi (2.5 M in THF, 0.46 mL, 1.15 mmol) in 5.0 mL of THF at −78° C., was added 4-bromo-2-chloropyridine (0.11 mL, 1.0 mmol) in 2.0 mL of THF slowly, and stirred for 2 hours. The solution of oxetan-3-one (93.6 mg, 1.3 mmol) in 2.0 mL of THF was added to the reaction at −78° C., and stirred another 30 minutes at this temperature. The reaction was quenched by sat. aq. NH₄Cl, and extracted with EtOAc. The combined organic extracts were dried (Na₂SO₄), concentrated in vacuo, followed by chromatography (EtOAC/Hexanes: 0-50%) to afford 3-(2-chloropyridin-4-yl)oxetan-3-ol. ESI-MS m/z 186.0 (MH⁺).

Step B: A mixture of 3-(2-chloropyridin-4-yl)oxetan-3-ol (10.0 mg, 0.05 mmol), 6-amino-8-(tert-butylamino)-2-(2-hydroxyethyl)-2,7-naphthyridin-1(2H)-one (14.0 mg, 0.05 mmol), Pd₂(dba)₃ (5.0 mg, 0.005 mmol), BINAP (3.1 mg, 0.005 mmol) and NaO^{t}Bu (10.0 mg, 0.11 mmol) in 0.5 mL of THF was degassed and purged with N₂, then heated at 85° C. for 45 minutes. The reaction mixture was purified on a preparation HPLC to afford 8-(tert-butylamino)-2-(2-hydroxyethyl)-6-(4-(3-hydroxyoxetan-3-yl)pyridin-2-ylamino)-2,7-naphthyridin-1(2H)-one. ESI-MS m/z 426.2 (MH⁺).

### Example 13 (Compound No. 612)

### Preparation of 6-(4-(2-amino-1-hydroxyethyl)pyridin-2-ylamino)-8-(tert-butylamino)-2-(2-hydroxyethyl)-2,7-naphthyridin-1(2H)-one

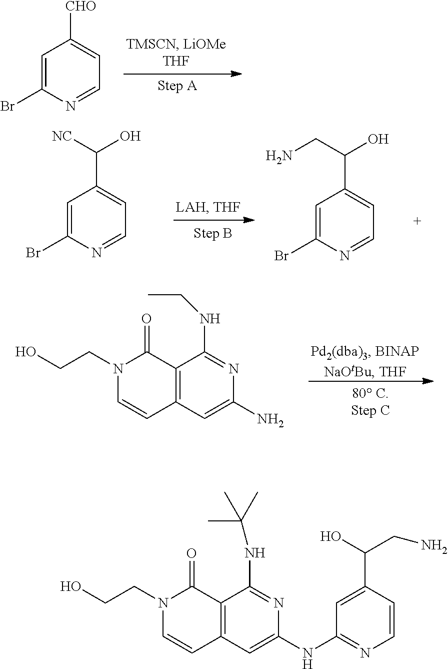

Step A: Under nitrogen, to a solution of LiOMe (1.0 N in MeOH, 0.25 mL, 0.25 mmol) in 10 mL of THF was added TMSCN (0.8 mL, 6.0 mmol), and the mixture was stirred at room temperature for 10 minutes. 2-bromoisonicotinaldehyde (925.0 mg, 5.0 mmol) was added to the reaction mixture, and the reaction mixture was stirred at room temperature overnight. The reaction was partitioned between EtOAc and sat. aq. NaHCO₃, the collected organic extracts were dried (Na₂SO₄), concentrated in vacuo, followed by chromatography (EtOAc/Hexanes: 0-50%) to afford 2-(2-bromopyridin-4-yl)-2-hydroxyacetonitrile. ESI-MS m/z 213.0 (MH⁺).

Step B: Under nitrogen, to a solution of 2-(2-bromopyridin-4-yl)-2-hydroxyacetonitrile (50.0 mg, 0.24 mmol) in 3.0 mL of THF was added LAH (2.0 M in THF, 0.47 mL, 0.96 mmol) slowly at 0° C. The reaction was stirred at this temperature for 2 hours. The reaction was quenched by 10% aq. NaOH, and extracted with EtOAc. The combined organic extracts were dried (Na₂SO₄), concentrated in vacuo, and the crude was used in the Step C directly.

Step C: A mixture of 2-amino-1-(2-bromopyridin-4-yl)ethanol (10.0 mg, 0.05 mmol), 6-amino-8-(ethylamino)-2-(2-hydroxyethyl)-2,7-naphthyridin-1(2H)-one (14.0 mg, 0.05 mmol), Pd₂(dba)₃ (5.0 mg, 0.005 mmol), BINAP (3.1 mg, 0.005 mmol) and NaO^{t}Bu (10.0 mg, 0.11 mmol) in 0.5 mL of THF was degassed and purged with N₂, then heated at 85° C. for 45 minutes. The reaction mixture was purified on a preparation HPLC to afford 6-(4-(2-amino-1-hydroxyethyl)pyridin-2-ylamino)-8-(tert-butylamino)-2-(2-hydroxyethyl)-2,7-naphthyridin-1(2H)-one. ESI-MS m/z 413.2 (MH⁺).

### Example 14 (Compound No. 618)

### Preparation of 8-(tert-butylamino)-2-(2-hydroxyethyl)-6-(6-vinylpyrimidin-4-ylamino)-2,7-naphthyridin-1(2H)-one

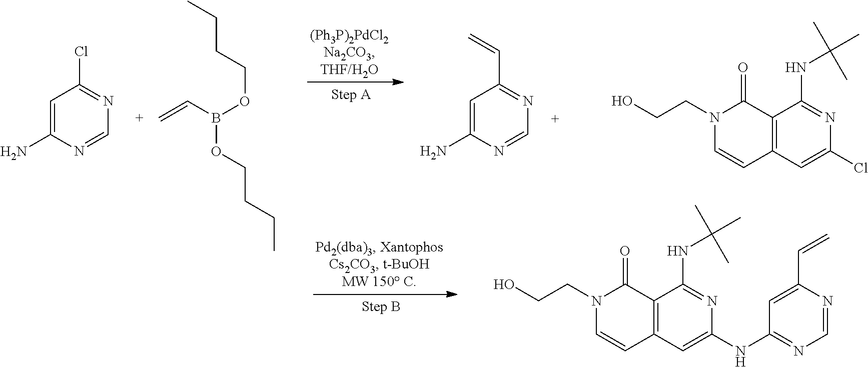

Step A: A mixture of 6-chloropyrimidin-4-amine (260.0 mg, 2.0 mmol), dibutyl vinylboronate (0.66 mL, 3.0 mmol), (Ph₃P)₂PdCl₂ (70.2 mg, 0.1 mmol), Na₂CO₃ (1.48 g, 14 mmol) in 8.0 mL of THF and 2.0 mL of H₂O was degassed and purged with nitrogen, then heated at 90° C. overnight. The reaction mixture was partitioned between EtOAc and brine, the combined organic extracts were dried (Na₂SO₄), concentrated in vacuo, followed by chromatography (EtOAc/Hexanes: 0-30%) to afford 6-vinylpyrimidin-4-amine. ESI-MS m/z 122.1 (MH⁺).

Step B: A mixture of 6-vinylpyrimidin-4-amine (51.1 mg, 0.27 mmol), 8-(tert-butylamino)-6-chloro-2-(2-hydroxyethyl)-2,7-naphthyridin-1(2H)-one (78.5 mg, 0.27 mmol), Pd₂(dba)₃ (24.0 mg, 0.027 mmol), Xantophos (15.4 mg, 0.027 mmol) and Cs₂CO₃ (259.0 mg, 0.80 mmol) in 2.0 mL of t-BuOH was degassed and purged with nitrogen, the mixture was heated at 150° C. in microwave for 30 minutes. The reaction mixture was purified on a preparation HPLC to afford 8-(tert-butylamino)-2-(2-hydroxyethyl)-6-(6-vinylpyrimidin-4-ylamino)-2,7-naphthyridin-1(2H)-one. ESI-MS m/z 381.2 (MH⁺).

### Example 15 (Compound No. 619)

### Preparation of 8-(tert-butylamino)-6-(6-ethylpyrimidin-4-ylamino)-2-(2-hydroxyethyl)-2,7-naphthyridin-1(2H)-one

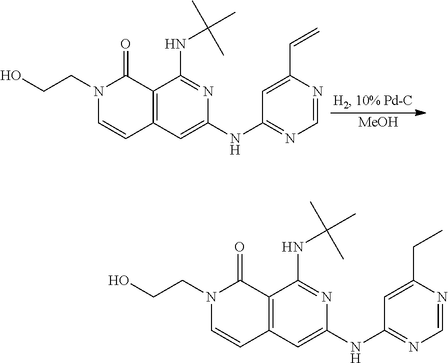

A mixture of 8-(tert-butylamino)-2-(2-hydroxyethyl)-6-(6-vinylpyrimidin-4-ylamino)-2,7-naphthyridin-1(2H)-one (38.0 mg, 0.1 mmol), 10% Pd—C (5.0 mg) in 10 mL of MeOH was vacuumed and purged with hydrogen overnight. The solid was filtered, and the filtrate was concentrated in vacuo to afford 8-(tert-butylamino)-6-(6-ethylpyrimidin-4-ylamino)-2-(2-hydroxyethyl)-2,7-naphthyridin-1(2H)-one. ESI-MS m/z 383.2 (MH⁺).

### Example 16 (Compound No. 630)

### Preparation of 8-(tert-butylamino)-6-(6-(3-hydroxy-3-methylazetidin-1-yl)pyrimidin-4-ylamino)-2-(2-hydroxyethyl)-2,7-naphthyridin-1(2H)-one

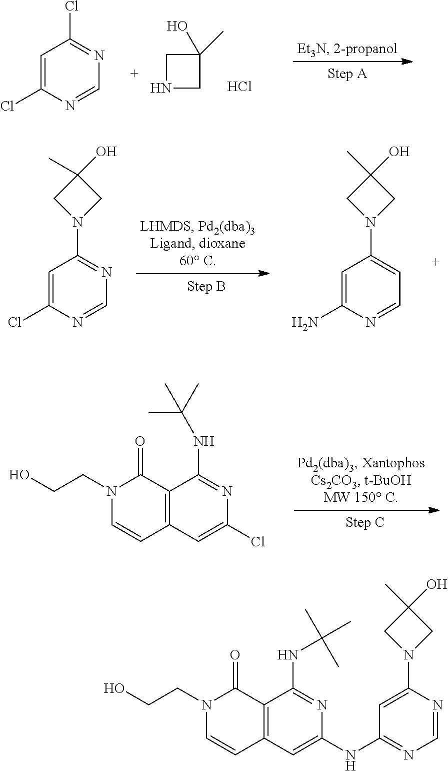

Step A: A mixture of 4,6-dichloropyrimidine (149.0 mg, 1.0 mmol), 3-methylazetidin-3-ol hydrochloride (147.6 mg, 1.2 mmol), Et₃N (0.33 mL, 2.4 mmol) in 3.0 mL of 2-propanol was heated to reflux 2 hours. The reaction mixture was concentrated in vacuo, and the crude was used directly in Step B.

Step B: A mixture of 1-(6-chloropyrimidin-4-yl)-3-methylazetidin-3-ol (80.0 mg, 0.38 mmol), LHMDS (1.0 N in THF, 1.1 mL, 1.1 mmol), Pd₂(dba)₃ (17.4 mg, 0.02 mmol), biphenyl-2-yl(cycloheptyl)(cyclohexyl)phosphine (13.3 mg, 0.04 mmol) in 1.0 mL of 1,4-dioxane was degassed and purged with N₂, then heated at 60° C. overnight. The reaction mixture was partitioned between EtOAc and brine, the combined organic extracts were dried (Na₂SO₄), concentrated in vacuo, followed by chromatography (MeOH/DCM: 0-10%) to afford 1-(2-aminopyridin-4-yl)-3-methylazetidin-3-ol.

Step C: A mixture of 1-(2-aminopyridin-4-yl)-3-methylazetidin-3-ol (51.1 mg, 0.27 mmol), 8-(tert-butylamino)-6-chloro-2-(2-hydroxyethyl)-2,7-naphthyridin-1(2H)-one (78.5 mg, 0.27 mmol), Pd₂(dba)₃ (24.0 mg, 0.027 mmol), Xantophos (15.4 mg, 0.027 mmol) and Cs₂CO₃ (259.0 mg, 0.80 mmol) in 2.0 mL of t-BuOH was degassed and purged with nitrogen, the mixture was heated at 150° C. in microwave for 30 min. The reaction mixture was purified on a preparation HPLC to afford 8-(tert-butylamino)-6-(6-(3-hydroxy-3-methylazetidin-1-yl)pyrimidin-4-ylamino)-2-(2-hydroxyethyl)-2,7-naphthyridin-1(2H)-one. ESI-MS m/z 440.2 (MH⁺).

### Example 17 (Compound No. 633)

### Preparation of 6-(6-acetylpyrimidin-4-ylamino)-8-(tert-butylamino)-2-(2-hydroxyethyl)-2,7-naphthyridin-1(2H)-one

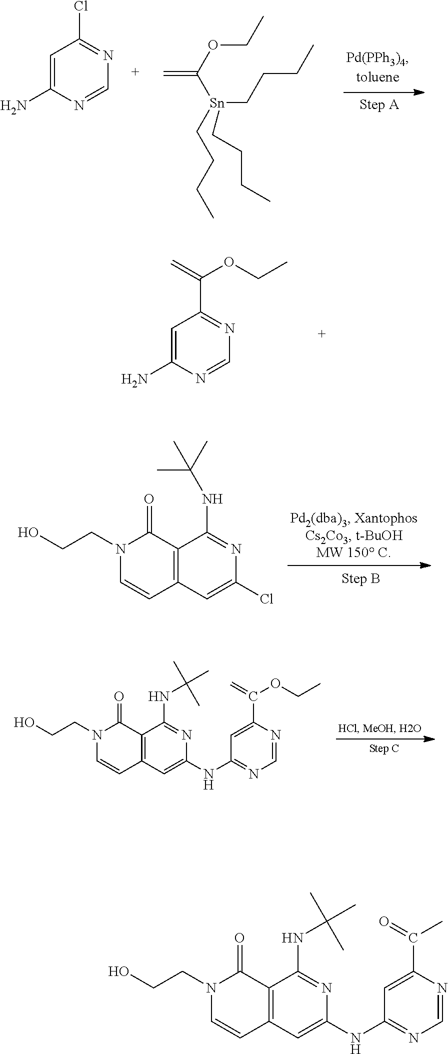

Step A: A mixture of 6-chloropyrimidin-4-amine (518.2 mg, 4.0 mmol), tributyl(1-ethoxyvinyl)stannane (1.35 mL, 4.0 mmol) and Pd (Ph₃P)₄ (92.5 mg, 0.08 mmol) in 20 mL of toluene was degassed, purged with nitrogen and heated at 110° C. overnight. The reaction was partitioned between EtOAc and brine, the combined organic extracts were dried (Na₂SO₄), concentrated in vacuo, followed by chromatography (MeOH/DCM: 0-10%) to afford 6-(1-ethoxyvinyl)pyrimidin-4-amine. ESI-MS 166.1 (MH⁺).

Step B: A mixture of 6-(1-ethoxyvinyl)pyrimidin-4-amine (51.1 mg, 0.27 mmol), 8-(tert-butylamino)-6-chloro-2-(2-hydroxyethyl)-2,7-naphthyridin-1(2H)-one (78.5 mg, 0.27 mmol), Pd₂(dba)₃ (24.0 mg, 0.027 mmol), Xantophos (15.4 mg, 0.027 mmol) and Cs₂CO₃ (259.0 mg, 0.80 mmol) in 2.0 mL of t-BuOH was degassed and purged with nitrogen, the mixture was heated at 150° C. in microwave for 30 minutes. The reaction mixture was purified on a preparation HPLC to afford 8-(tert-butylamino)-6-(6-(1-ethoxyvinyl)pyrimidin-4-ylamino)-2-(2-hydroxyethyl)-2,7-naphthyridin-1(2H)-one.

Step C: A mixture of 8-(tert-butylamino)-6-(6-(1-ethoxyvinyl)pyrimidin-4-ylamino)-2-(2-hydroxyethyl)-2,7-naphthyridin-1(2H)-one (188.2 mg, 0.44 mmol), aq. 1N HCl (2.2 mL, 2.2 mmol) in 3.0 mL of MeOH was stirred at room temperature overnight. The reaction mixture was concentrated in vacuo, the resulting crude was neutralized with sat. aq. NaHCO₃, the extracted with EtOAc, and then washed by brine. The combined organic extracts were dried (Na₂SO₄), concentrated in vacuo to afford 6-(6-acetylpyrimidin-4-ylamino)-8-(tert-butylamino)-2-(2-hydroxyethyl)-2,7-naphthyridin-1(2H)-one. ESI-MS m/z 397.2 (MH⁺).

### Example 18 (Compound No. 635)

### Preparation of 8-(tert-butylamino)-2-(2-hydroxyethyl)-6-(6-(3-(trifluoromethyl)-1H-pyrazol-4-yl)pyrimidin-4-ylamino)-2,7-naphthyridin-1(2H)-one

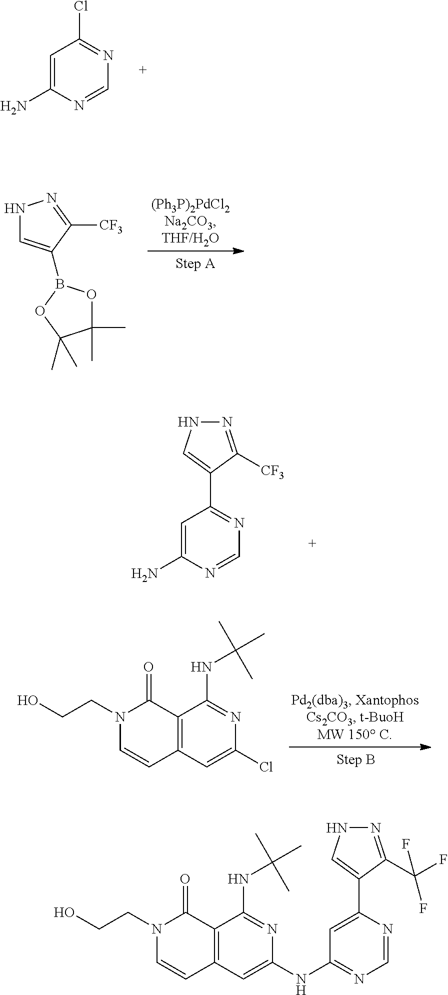

Step A: A mixture of 6-chloropyrimidin-4-amine (260.0 mg, 2.0 mmol), 4-(4,4,5,5-tetramethyl-1,3,2-dioxaborolan-2-yl)-3-(trifluoromethyl)-1H-pyrazole (0.66 mL, 3.0 mmol), (Ph₃P)₂PdCl₂ (70.2 mg, 0.1 mmol), Na₂CO₃ (1.48 g, 14 mmol) in 8.0 mL of THF and 2.0 mL of H₂O was degassed and purged with nitrogen, then heated at 90° C. overnight. The reaction mixture was partitioned between EtOAc and brine, the combined organic extracts were dried (Na₂SO₄), concentrated in vacuo, followed by chromatography (EtOAc/Hexanes: 0-30%) to afford 6-(3-(trifluoromethyl)-1H-pyrazol-4-yl)pyrimidin-4-amine. ESI-MS m/z 230.1 (MH⁺).

Step B: A mixture of 6-(3-(trifluoromethyl)-1H-pyrazol-4-yl)pyrimidin-4-amine (51.1 mg, 0.27 mmol), 8-(tert-butylamino)-6-chloro-2-(2-hydroxyethyl)-2,7-naphthyridin-1(2H)-one (78.5 mg, 0.27 mmol), Pd₂(dba)₃ (24.0 mg, 0.027 mmol), Xantophos (15.4 mg, 0.027 mmol) and Cs₂CO₃ (259.0 mg, 0.80 mmol) in 2.0 mL of t-BuOH was degassed and purged with nitrogen, the mixture was heated at 150° C. in microwave for 30 minutes. The reaction mixture was purified on a preparation HPLC to afford 8-(tert-butylamino)-2-(2-hydroxyethyl)-6-(6-(3-(trifluoromethyl)-1H-pyrazol-4-yl)pyrimidin-4-ylamino)-2,7-naphthyridin-1(2H)-one. ESI-MS m/z 489.2 (MH⁺).

### Example 19 (Compound No. 643)

### Preparation of 8-(tert-butylamino)-2-((S)-2,3-dihydroxypropyl)-6-(6-((S)-1-hydroxypropan-2-yloxy)pyrimidin-4-ylamino)-2,7-naphthyridin-1(2H)-one

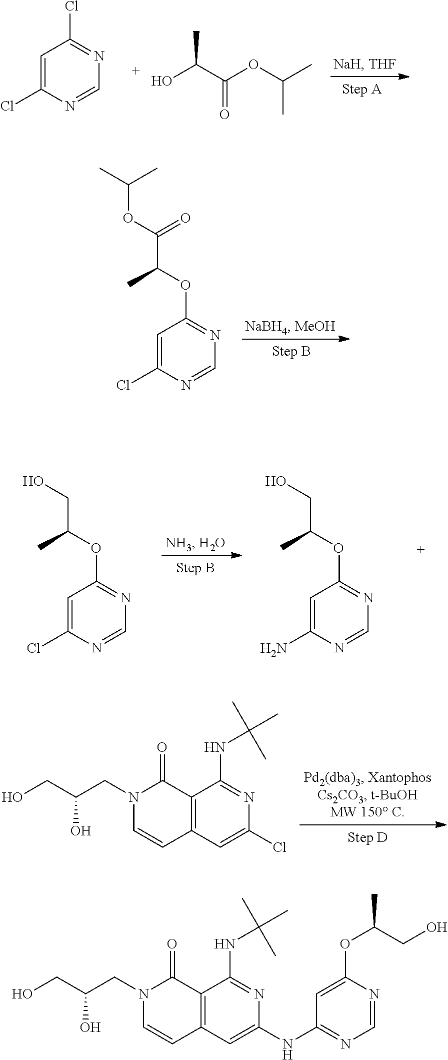

Step A: Under nitrogen, to a suspension of NaH (127.2 mg, 3.2 mmol) in 10 mL of THF was added (S)-isopropyl 2-hydroxypropanoate (422.4 mg, 3.2 mmol) at 0° C., and stirred 10 minutes. 4,6-dichloropyrimidine (446.9 mg, 3.0 mmol) was added to the reaction, and the reaction was warmed to room temperature, and stirred for another 2.0 hours. The reaction was partitioned between EtOAc and brine, the combined organic extracts were dried (Na₂SO₄), concentrated in vacuo, followed by chromatography (EtOAc/Hexanes: 0-30%) to afford (S)-isopropyl 2-(6-chloropyrimidin-4-yloxy)propanoate. ESI-MS m/z 245.1 (MH⁺).

Step B: To a solution of (S)-isopropyl 2-(6-chloropyrimidin-4-yloxy)propanoate (30.0 mg, 0.2 mmol) in 0.5 mL of MeOH was added NaBH₄(38.0 mg, 1.0 mmol), and the reaction was heated to reflux for 3 hours to afford (S)-2-(6-chloropyrimidin-4-yloxy)propan-1-ol. ESI-MS m/z 189.0 (MH⁺).

Step C: A mixture of (S)-2-(6-chloropyrimidin-4-yloxy)propan-1-ol (188.0 mg, 1.0 mmol) in 3 mL of ammonium hydroxide was heated at 100° C. overnight. The reaction was concentrated in vacuo, and the crude mixture of (S)-2-(6-aminopyrimidin-4-yloxy)propan-1-ol was used directly in next step.

Step D: A mixture of (S)-2-(6-aminopyrimidin-4-yloxy)propan-1-ol (51.1 mg, 0.27 mmol), (S)-8-(tert-butylamino)-6-chloro-2-(2,3-dihydroxypropyl)-2,7-naphthyridin-1(2H)-one (78.5 mg, 0.27 mmol), Pd₂(dba)₃ (24.0 mg, 0.027 mmol), Xantophos (15.4 mg, 0.027 mmol) and Cs₂CO₃ (259.0 mg, 0.80 mmol) in 2.0 mL of t-BuOH was degassed and purged with nitrogen, the mixture was heated at 150° C. in microwave for 30 minutes. The reaction mixture was purified on a preparation HPLC to afford 8-(tert-butylamino)-2-((S)-2,3-dihydroxypropyl)-6-(64(S)-1-hydroxypropan-2-yloxy)pyrimidin-4-ylamino)-2,7-naphthyridin-1(2H)-one. ESI-MS m/z 459.2 (MH⁺).

### Example 20 (Compound No. 645)

### Preparation of 8-(tert-butylamino)-2-(2-hydroxyethyl)-6-(6-(piperidin-4-yloxy)pyrimidin-4-ylamino)-2,7-naphthyridin-1(2H)-one

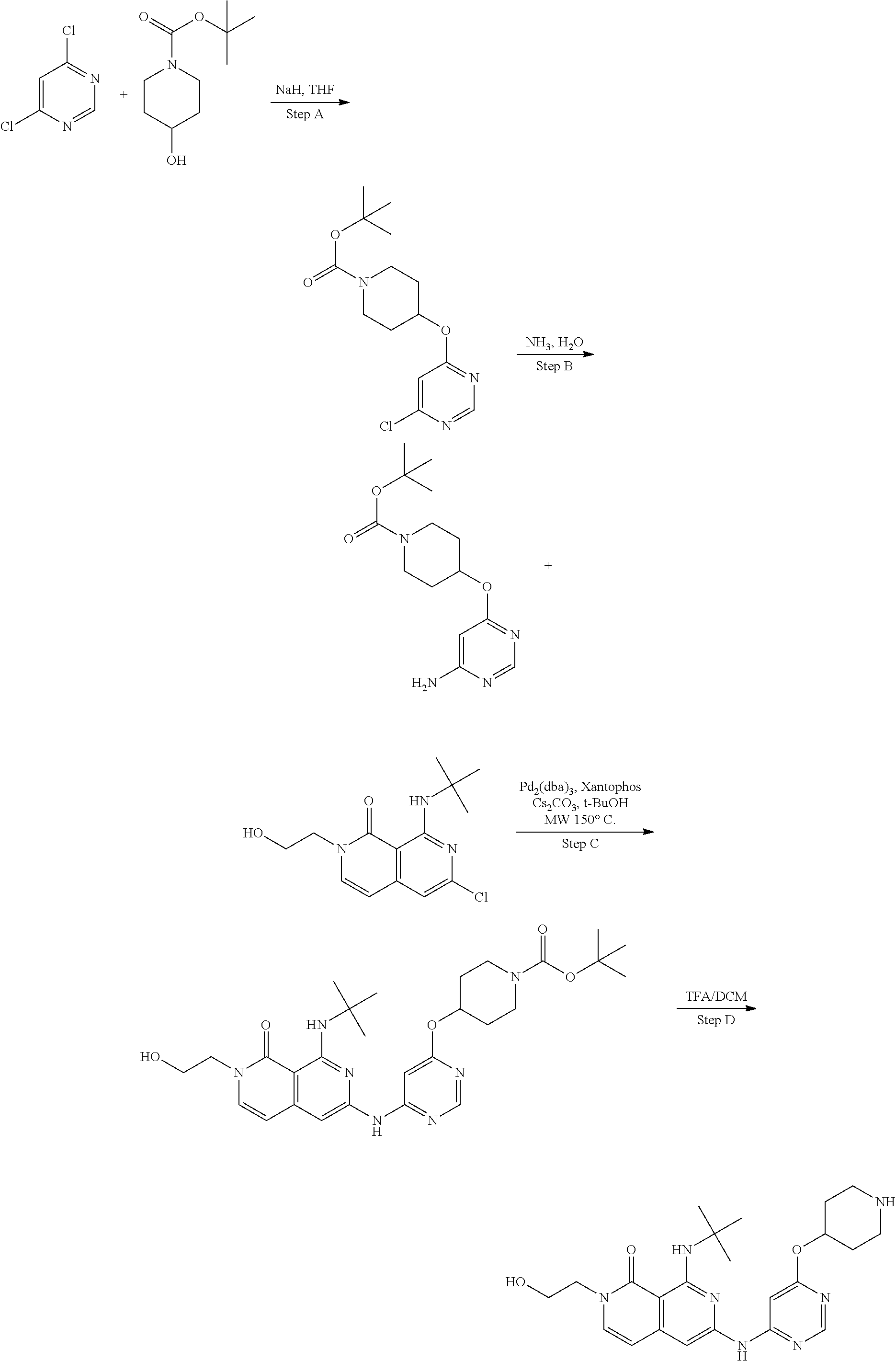

Step A: Under nitrogen, to a suspension of NaH (127.2 mg, 3.2 mmol) in 10 mL of THF was added tert-butyl 4-hydroxypiperidine-1-carboxylate (422.4 mg, 3.2 mmol) at 0° C., and stirred 10 minutes. 4,6-dichloropyrimidine (446.9 mg, 3.0 mmol) was added to the reaction, and the reaction was warmed to room temperature, and stirred for another 2.0 hours. The reaction was partitioned between EtOAc and brine, the combined organic extracts were dried (Na₂SO₄), concentrated in vacuo, followed by chromatography (EtOAc/Hexanes: 0-30%) to afford tert-butyl 4-(6-chloropyrimidin-4-yloxy)piperidine-1-carboxylate. ESI-MS m/z 314.1 (MH⁺).

Step B: A mixture of tert-butyl 4-(6-chloropyrimidin-4-yloxy)piperidine-1-carboxylate (188.0 mg, 1.0 mmol) in 3 mL of ammonium hydroxide was heated at 100° C. overnight. The reaction was concentrated in vacuo to afford tert-butyl 4-(6-aminopyrimidin-4-yloxy)piperidine-1-carboxylate. ESI-MS m/z 295.2 (MH⁺).

Step C: A mixture of tert-butyl 4-(6-aminopyrimidin-4-yloxy)piperidine-1-carboxylate (51.1 mg, 0.27 mmol), 8-(tert-butylamino)-6-chloro-2-(2-hydroxyethyl)-2,7-naphthyridin-1(2H)-one (78.5 mg, 0.27 mmol), Pd₂(dba)₃ (24.0 mg, 0.027 mmol), Xantophos (15.4 mg, 0.027 mmol) and Cs₂CO₃ (259.0 mg, 0.80 mmol) in 2.0 mL of t-BuOH was degassed and purged with nitrogen, the mixture was heated at 150° C. in microwave for 30 minutes. The reaction mixture was purified on a preparation HPLC to afford tert-butyl 4-(6-(1-(tert-butylamino)-7-(2-hydroxyethyl)-8-oxo-7,8-dihydro-2,7-naphthyridin-3-ylamino)pyrimidin-4-yloxy)piperidine-1-carboxylate. ESI-MS m/z 554.3 (MH⁺).

Step D: A mixture of tert-butyl 4-(6-(1-(tert-butylamino)-7-(2-hydroxyethyl)-8-oxo-7,8-dihydro-2,7-naphthyridin-3-ylamino)pyrimidin-4-yloxy)piperidine-1-carboxylate (27.0 mg, 0.05 mmol), 10 mL of 10% TFA in DCM was stirred at room temperature 5.0 hours. The mixture was concentrated in vacuo, and purified on preparation HPLC to afford 8-(tert-butylamino)-2-(2-hydroxyethyl)-6-(6-(piperidin-4-yloxy)pyrimidin-4-ylamino)-2,7-naphthyridin-1(2H)-one. ESI-MS m/z 454.3 (MH⁺).

### Example 21 (Compound No. 647)

### Preparation of 8-(tert-butylamino)-2-(2-hydroxyethyl)-6-(6-(1-(2-methoxyethyl)piperidin-4-yloxy)pyrimidin-4-ylamino)-2,7-naphthyridin-1(2H)-one

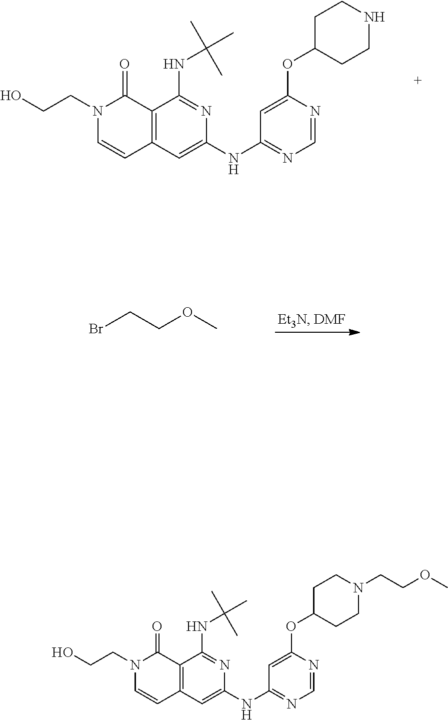

A mixture of 8-(tert-butylamino)-2-(2-hydroxyethyl)-6-(6-(piperidin-4-yloxy)pyrimidin-4-ylamino)-2,7-naphthyridin-1(2H)-one (50.0 mg, 0.11 mmol), 1-bromo-2-methoxyethane (76.5 mg, 0.55 mmol) in 2 mL of DMF was heated at 80° C. for 4.0 h. The mixture was applied on Preparation HPLC to afford 8-(tert-butylamino)-2-(2-hydroxyethyl)-6-(6-(1-(2-methoxyethyl)piperidin-4-yloxy)pyrimidin-4-ylamino)-2,7-naphthyridin-1(2H)-one. ESI-MS m/z 512.3 (MH⁺).

### Example 22 (Compound No. 648)

### Preparation of 8-(tert-butylamino)-2-(2-hydroxyethyl)-6-(6-(1-methylpiperidin-4-yloxy)pyrimidin-4-ylamino)-2,7-naphthyridin-1(2H)-one

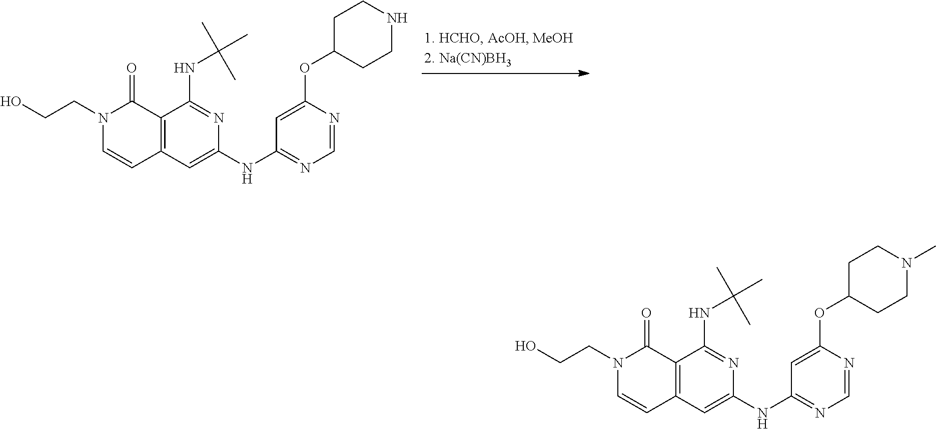

To a mixture of 8-(tert-butylamino)-2-(2-hydroxyethyl)-6-(6-(piperidin-4-yloxy)pyrimidin-4-ylamino)-2,7-naphthyridin-1(2H)-one (62.5 mg, 0.14 mmol) in 2.0 mL of MeOH was added 1 drop of AcOH, and formaldehyde (37% in water, 17.0 mg, 0.21 mmol) sequentially. The reaction was stirred at room temperature 30 minutes, followed by adding Na(CN)BH₃ (12.0 mg, 0.21 mmol), and stirred another 1.0 hour. The reaction was quenched by sat. aq. NH₄Cl, then extracted with EtOAc. The combined organic extracts were dried (Na₂SO₄), concentration in vauo, followed by purification on preparation HPLC to afford t8-(tert-butylamino)-2-(2-hydroxyethyl)-6-(6-(1-methylpiperidin-4-yloxy)pyrimidin-4-ylamino)-2,7-naphthyridin-1(2H)-one. ESI-MS m/z 468.3 (MH⁺).

### Example 23 (Compound No. 664)

### Synthesis of 8-(tert-butylamino)-2-(2,3-dihydroxypropyl)-6-(pyrazin-2-ylamino)-2,7-naphthyridin-1(2H)-one

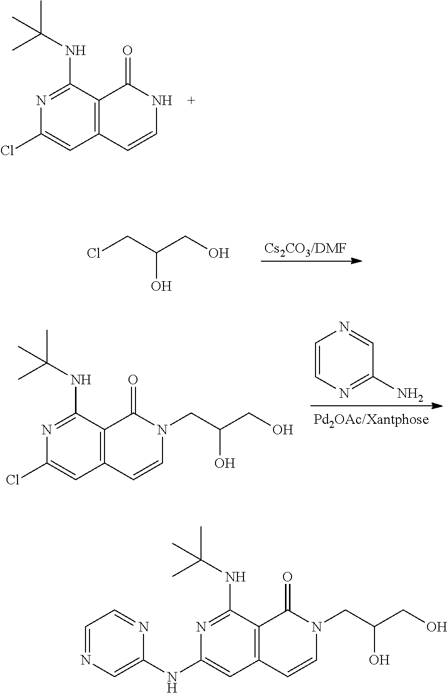

8-(tert-butylamino)-6-chloro-2,7-naphthyridin-1(2H)-one (100 mg, 0.4 mmol) was added in a suspension of Cs₂CO₃ (388 mg, 1.2 mmol) in DMF (1.5 mL). The solution was stirred for 30 minutes and 3-chloropropane-1,2-diol (87.6 mg, 0.8 mmol) was added into the solution. Stirring was continued for 30 hours and the mixture was then poured into ice cold water. The reaction mixture was worked up with ethyl acetate and purified by silica gel column chromatography by using DCM:MeOH (10:1) as eluent to give 8-(tert-butylamino)-6-chloro-2-(2,3-dihydroxypropyl)-2,7-naphthyridin-1(2H)-one. ESI-MS (m/z) 326.12 (MH⁺).

1.0 mL dioxane was added to a mixture of 8-(tert-butylamino)-6-chloro-2-(2,3-dihydroxypropyl)-2,7-naphthyridin-1(2H)-one (52 mg, 0.16 mmol), 2-aminopyrazine (30.4 mg, 0.32 mmol), Pd₂OAc (3.5 mg, 10%), Xantphos (13.6 mg, 20%) and Cs₂CO₃ (156.4 mg, 0.48 mmol). The mixture was heated at 150° C. for 3 hours using an oil bath. The mixture was then filtered and purified by HPLC to give 8-(tert-butylamino)-2-(2,3-dihydroxypropyl)-6-(pyrazin-2-ylamino)-2,7-naphthyridin-1(2H)-one. ¹H NMR (MeOH-d₄) δ 8.11 (d, J=1.6 Hz, 1H), 7.45 (dd, J=1.6, 2.8 Hz, 1H), 7.21 (d, J=2.8 Hz, 1H), 6.45 (d, J=7.6 Hz, 1H), 6.05 (s, 1H), 5.46 (d, J=7.6 Hz, 1H), 3.40 (m, 1H), 3.16 (m, 1H), 2.91 (m, 1H), 2.75 (m, 2H), 2.51 (m, 1H), 1.75 (s, 9H). ESI-MS m/z 385.2 (MH⁺).

### Example 24 (Compound No. 668)

### Synthesis of (R)-8-(tert-butylamino)-2-(2,3-dihydroxypropyl)-6-(pyrazin-2-ylamino)-2,7-naphthyridin-1(2H)-one

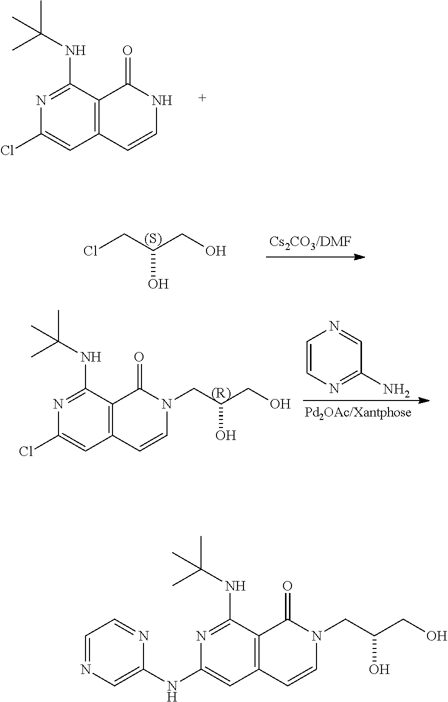

8-(tert-butylamino)-6-chloro-2,7-naphthyridin-1(2H)-one (3 g, 12 mmol) was added in a suspension of Cs₂CO₃ (11.5 g, 36 mmol) in DMF (25 mL). The solution was stirred for 30 minutes and (S)-3-chloropropane-1,2-diol (2.64 g, 24 mmol) was added into the solution. The stiffing was continued for 30 hours and poured into ice cold water. The reaction mixture was worked up with ethyl acetate and purified by silica gel column chromatography by using DCM:MeOH (10:1) as eluent to obtain (R)-8-(tert-butylamino)-6-chloro-2-(2,3-dihydroxypropyl)-2,7-naphthyridin-1(2H)-one. ¹H NMR (MeOH-d₄) δ 9.70 (s, 1H), 7.40 (d, J=7.2, 1H), 6.84 (s, 1H), 6.30 (d, J=7.2 Hz, 1H), 4.28 (m, 1H), 3.96 (m, 1H), 3.73 (m, 1H), 3.55 (m, 2H), 3.31 (m, 1H), 1.50 (s, 9H), ESI-MS (m/z) 326.12 (MH⁺).

10 mL dioxane was added to a mixture of (R)-8-(tert-butylamino)-6-chloro-2-(2,3-dihydroxypropyl)-2,7-naphthyridin-1(2H)-one (1 g, 3.0 mmol), 2-aminopyrazine (304 mg, 3.2 mmol), Pd(OAc)₂ (67 mg, 10%), Xantphos (260 mg, 15%) and Cs₂CO₃ (2 g, 6 mmol). The mixture was subjected to microwave irradiation at 150° C. for 20 minutes. The mixture was then filtered and washed with ethyl acetate repeatedly. The combined organic solvents was washed with brine and then with water, and then purified by silica gel column chromatography using DCM/MeOH (20:1) as eluent to give (R)-8-(tert-butylamino)-2-(2,3-dihydroxypropyl)-6-(pyrazin-2-ylamino)-2,7-naphthyridin-1(2H)-one, which was then crystallized from ethylacetate/hexanes. ¹H NMR (MeOH-d₄) δ 8.92 (d, J=1.6 Hz, 1H), 8.23 (dd, J=1.6, 2.8 Hz, 1H), 8.0 (d, J=2.8 Hz, 1H), 7.25 (d, J=7.6 Hz, 1H), 6.84 (s, 1H), 6.26 (d, J=7.6 Hz, 1H), 4.21 (m, 1H), 3.97 (m, 1H), 3.71 (m, 1H), 3.55 (m, 2H), 3.16 (m, 1H), 1.56 (s, 9H). ESI-MS m/z 385.2 (MH⁺)

### Example 25 (Compound No. 685)

### Preparation of (E)-8-(tert-butylamino)-2-(2-hydroxyethyl)-6-(4-(1-(hydroxyimino)ethyl)pyridin-2-ylamino)-2,7-naphthyridin-1(2H)-one

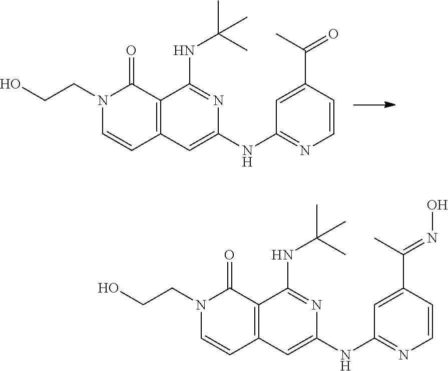

A mixture of 6-(4-acetylpyridin-2-ylamino)-8-(tert-butylamino)-2-(2-hydroxyethyl)-2,7-naphthyridin-1(2H)-one (25.0 mg, 0.063 mmol), hydroxylamine hydrochloride (8.8 mg, 0.126 mmol) and NaOAc (15.5 mg, 0.158 mmol) in MeOH (1 mL) was heated at 60° C. overnight, cooled down, quenched with H₂O and extracted with EtOAc. The combined organic layer was evaporated under reduced pressure and purified by preparatory LC/MS to provide the title compound; ESI-MS m/z 411.2 (MH⁺).

### Example 26 (Compound No. 690)

### Preparation of 6-(4-(1H-pyrazol-5-yl)pyridin-2-ylamino)-8-(tert-butylamino)-2-(2-hydroxyethyl)-2,7-naphthyridin-1(2H)-one

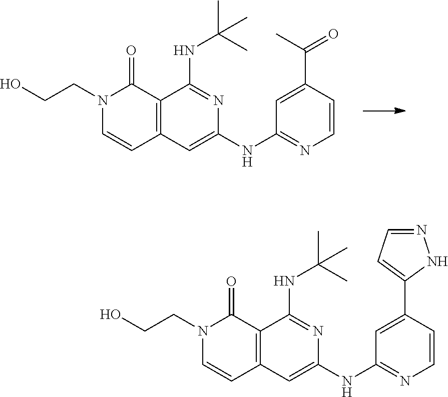

A solution of 6-(4-acetylpyridin-2-ylamino)-8-(tert-butylamino)-2-(2-hydroxyethyl)-2,7-naphthyridin-1(2H)-one (27.5 mg, 0.07 mmol) in N,N-dimethylformamide dimethyl acetal (0.5 mL) was heated at 80° C. overnight. After removal of solvent under reduced pressure, the residue was taken in MeOH (0.5 mL). Hydrazine monohydrate (6.79 μL, 0.14 mmol) was added. The resulted mixture was heated at 80° C. for 2 hours, evaporated under reduced pressure and purified by preparatory LC/MS to provide title compound; ESI-MS m/z 420.2 (MH⁺).

### Example 27 (Compound No. 698)

### Preparation of 8-(tert-butylamino)-2-(2-hydroxyethyl)-6-(4-(2-hydroxypropan-2-yl)pyridin-2-ylamino)-2,7-naphthyridin-1(2H)-one

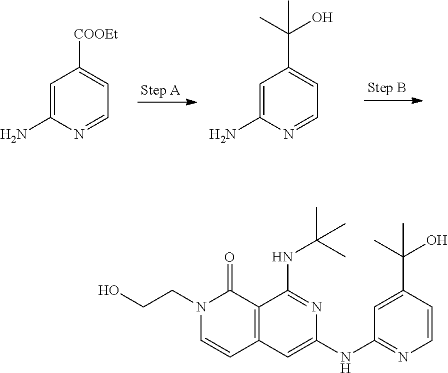

Step A: To a MeMgCl solution (3.0 M in THF, 10 mL, 30 mmol) in a dry flask was added dropwise a solution of ethyl 2-aminoisonicotinate (498.6 mg, 3.0 mmol) in anhydrous THF (10 mL) at 0° C. The mixture was stirred at 0° C. for 30 minutes before being warmed up to room temperature. The mixture was stirred at room temperature for another 30 minutes, poured into cold saturated aqueous NH₄Cl solution (100 mL), and extracted with EtOAc (3×50 mL). The combined organic layer was dried over MgSO₄, concentrated, and purified by silica gel chromatography (eluent: 0-10% MeOH in DCM) to provide 2-(2-aminopyridin-4-yl)propan-2-ol as a yellow solid.

Step B: To a solution of 8-(tert-butylamino)-6-chloro-2-(2-hydroxyethyl)-2,7-naphthyridin-1(2H)-one (29.6 mg, 0.10 mmol) in 1,4-dioxane (1 mL) were added 2-(2-aminopyridin-4-yl)propan-2-ol (16.7 mg, 0.11 mmol), Cs₂CO₃ (130.3 mg, 0.40 mmol), and a catalytic amount of Pd(dba)₃ and Xantphos. The reaction mixture was purged with N₂ and heated at 150° C. by a microwave reactor for 30 minutes. The mixture was then cooled, quenched with H₂O and extracted with EtOAc. The combined organic layer was dried over MgSO₄, concentrated, and purified by silica gel chromatography (eluent: 0-10% MeOH in DCM) to provide 8-(tert-butylamino)-2-(2-hydroxyethyl)-6-(4-(2-hydroxypropan-2-yl)pyridin-2-ylamino)-2,7-naphthyridin-1(2H)-one as a yellow solid; ¹H NMR (CD₃OD, 400 MHz) δ 8.16 (d, T=5.6 Hz, 1H), 7.49 (d, J=1.6 Hz, 1H), 7.22 (d, J=7.2 Hz, 1H), 7.03 (dd, J=5.6, 1.6 Hz, 1H), 6.83 (s, 1H), 6.25 (d, J=7.2 Hz, 1H), 3.98 (t, T=5.2 Hz, 2H), 3.81 (t, T=5.2 Hz, 2H), 1.55 (s, 9H), 1.53 (s, 6H); ESI-MS m/z 412.2 (MH⁺).

### Example 28 (Compound No. 700)

### Preparation of 8-(tert-butylamino)-2-(2-hydroxyethyl)-6-(4-(2-hydroxypropan-2-yl)pyridin-2-ylamino)-2,7-naphthyridin-1(2H)-one

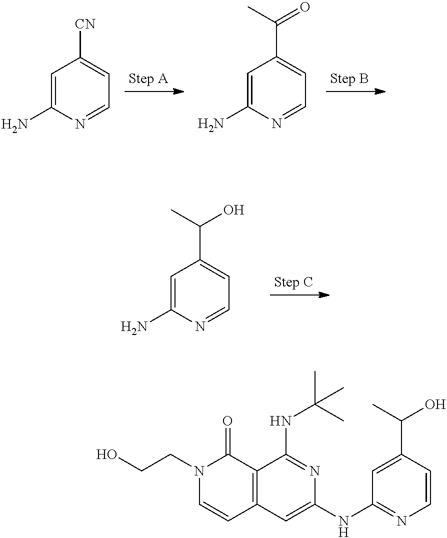

Step A: To a solution of 2-aminoisonicotinonitrile (119.1 mg, 1.0 mmol) in Et₂O (2 mL) was added dropwise a MeMgBr solution (3.0 M in Et₂O, 2 mL, 6.0 mmol) at 0° C. The mixture was refluxed overnight, cooled down, quenched with cold H₂O, neutralized with concentrated HCl at 0° C., and extracted with EtOAc. The combined organic layer was evaporated under reduced pressure to provide crude 1-(2-aminopyridin-4-yl)ethanone.

Step B: To a solution of crude 1-(2-aminopyridin-4-yl)ethanone (estimate 1.0 mmol) in MeOH (5 mL) was added NaBH₄ (75.7 mg, 2.0 mmol). The mixture was stirred at room temperature overnight, quenched with cold H₂O, and extracted with EtOAc. The combined organic layer was evaporated under reduced pressure to provide crude 1-(2-aminopyridin-4-yl)ethanol.

Step C: To a solution of 8-(tert-butylamino)-6-chloro-2-(2-hydroxyethyl)-2,7-naphthyridin-1(2H)-one (29.6 mg, 0.10 mmol) in 1,4-dioxane (1 mL) were added crude 1-(2-aminopyridin-4-yl)ethanol (estimate 0.20 mmol), Cs₂CO₃ (130.3 mg, 0.40 mmol), and a catalytic amount of Pd(dba)₃ and Xantphos. The reaction mixture was purged with N₂ and heated at 100° C. overnight. The mixture was then cooled, quenched with H₂O and extracted with EtOAc. The combined organic layer was concentrated and purified by preparatory LC/MS to provide title compound; ¹H NMR (CD₃OD, 400 MHz) δ 8.16 (d, J=5.6 Hz, 1H), 7.69 (s, 1H), 7.23 (d, J=7.6 Hz, 1H), 6.97 (d, J=5.6 Hz, 1H), 6.59 (s, 1H), 6.24 (d, J=7.6 Hz, 1H), 4.81 (q, J=6.4 Hz, 1H), 3.99 (t, J=5.2 Hz, 2H), 3.81 (t, J=5.2 Hz, 2H), 1.57 (s, 9H), 1.45 (d, J=6.4 Hz, 3H); ESI-MS m/z 398.2 (MH⁺).

### Example 29 (Compound No. 711)

### Preparation of 6-(4-(2-aminopropan-2-yl)pyridin-2-ylamino)-8-(tert-butylamino)-2-(2-hydroxyethyl)-2,7-naphthyridin-1(2H)-one

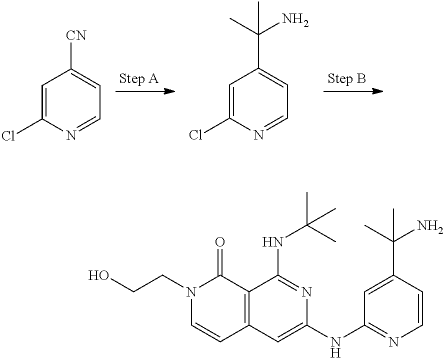

Step A: To a solution of 2-chloroisonicotinonitrile (138.6 mg, 1.0 mmol) in Et₂O (5 mL) was slowly added a MeMgCl solution (3.0 M in Et₂₀, 1 mL, 3 mmol) at 0° C. The mixture was stirred at room temperature for 30 minutes before Ti(OⁱPr)₄ (293 μL, 1.0 mmol) was added. The mixture was refluxed overnight, cooled down, quenched with 1N NaOH aqueous solution (10 mL), and extracted with Et₂O (3×10 mL). The combined organic layer was evaporated under reduced pressure to provide crude 2-(2-chloropyridin-4-yl)propan-2-amine, which was used in next step without further purification.

Step B: To a solution of 6-amino-8-(tert-butylamino)-2-(2-hydroxyethyl)-2,7-naphthyridin-1(2H)-one (20.0 mg, 0.072 mmol) in THF (1 mL) were added crude 2-(2-chloropyridin-4-yl)propan-2-amine (estimate 0.3 mmol), NaO^{t}Bu (13.9 mg, 0.144 mmol), and a catalytic amount of Pd₂(dba)₃ and BINAP. The reaction mixture was purged with N₂ and heated at 80° C. for one hour. The mixture was then cooled, quenched with H₂O and extracted with EtOAc. The combined organic layer was evaporated under reduced pressure and purified by preparatory LC/MS to provide 6-(4-(2-aminopropan-2-yl)pyridin-2-ylamino)-8-(tert-butylamino)-2-(2-hydroxyethyl)-2,7-naphthyridin-1(2H)-one; ESI-MS m/z 411.2 (MH⁺).

### Example 30 (Compound No. 712)

### Preparation of 6-(4-(1-aminoethyl)pyridin-2-ylamino)-8-(tert-butylamino)-2-(2-hydroxyethyl)-2,7-naphthyridin-1(2H)-one

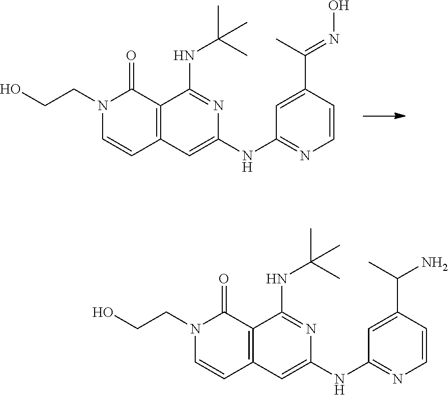

To a solution of (E)-8-(tert-butylamino)-2-(2-hydroxyethyl)-6-(4-(1-(hydroxyimino)ethyl)pyridin-2-ylamino)-2,7-naphthyridin-1(2H)-one (5.0 mg, 0.012 mmol) in MeOH (0.5 mL) were added HCl solution (4N in 1,4-dioxane, 1 drop) and a catalytic amount of Pd/C. The reaction mixture was stirred under hydrogen balloon at room temperature overnight. After removal of Pd/C by filtration, the filtrate was concentrated and purified by preparatory LC/MS to provide 6-(4-(1-aminoethyl)pyridin-2-ylamino)-8-(tert-butylamino)-2-(2-hydroxyethyl)-2,7-naphthyridin-1(2H)-one; ESI-MS m/z 397.2 (MH⁺).

### Example 31 (Compound No. 738)

### Preparation of 8-(tert-butylamino)-2-(2-(methylsulfonyl)ethyl)-6-(pyrazin-2-ylamino)-2,7-naphthyridin-1(2H)-one

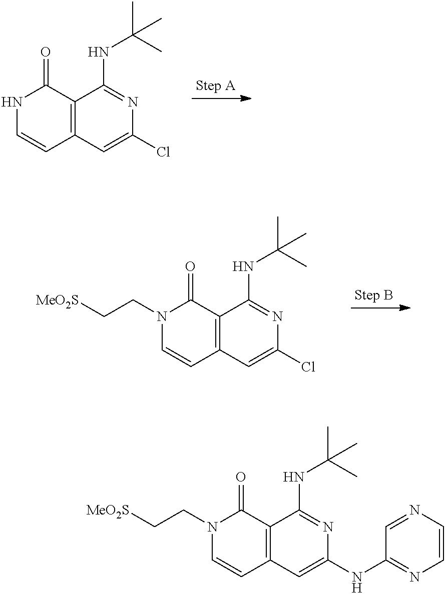

Step A: A mixture of 8-(tert-butylamino)-6-chloro-2,7-naphthyridin-1(2H)-one (100.7 mg, 0.40 mmol), methyl vinyl sulfone (63.1 μL 0.72 mmol) and Cs₂CO₃ (260.7 mg, 0.80 mmol) in DMF (2 mL) was stirred at room temperature overnight. The mixture was diluted with H₂O and extracted with EtOAc. The combined organic layer was concentrated and purified by silica gel chromatography (eluent: 0-70% EtOAc in hexanes) to provide 8-(tert-butylamino)-6-chloro-2-(2-(methylsulfonyl)ethyl)-2,7-naphthyridin-1(2H)-one as a white solid (116.5 mg, 81% yield).

Step B: To a solution of 8-(tert-butylamino)-6-chloro-2-(2-(methylsulfonyl)ethyl)-2,7-naphthyridin-1(2H)-one (50.0 mg, 0.14 mmol) in 1,4-dioxane (1 mL) were added aminopyrazine (14.6 mg, 0.154 mmol), Na₂CO₃ (59.4 mg, 0.56 mmol), and a catalytic amount of Pd(dba)₃ and Xantphos. The reaction mixture was purged with N₂ and heated at 120° C. overnight, cooled, quenched with H₂O and extracted with EtOAc. The combined organic layer was dried over MgSO₄, concentrated, and purified by silica gel chromatography (eluent: 0-3% MeOH in DCM) to provide 8-(tert-butylamino)-2-(2-(methylsulfonyl)ethyl)-6-(pyrazin-2-ylamino)-2,7-naphthyridin-1(2H)-one as a white solid; ¹H NMR (CD₃OD, 400 MHz) δ 8.94 (s, 1H), 8.27 (d, J=2.8 Hz, 1H), 8.03 (d, J=2.8 Hz, 1H), 7.32 (d, J=7.6 Hz, 1H), 6.87 (s, 1H), 6.30 (d, J=7.6 Hz, 1H), 4.33 (t, J=6.8 Hz, 2H), 3.60 (t, J=6.8 Hz, 2H), 3.01 (s, 3H), 1.56 (s, 9H); ESI-MS m/z 417.2 (MH⁺).

### Example 32 (Compound No. 744)

### N-(3-(2-Aminopyrimidin-5-yl)-7-ethyl-8-oxo-7,8-dihydro-2,7-naphthyridin-1-yl)methane sulfonamide

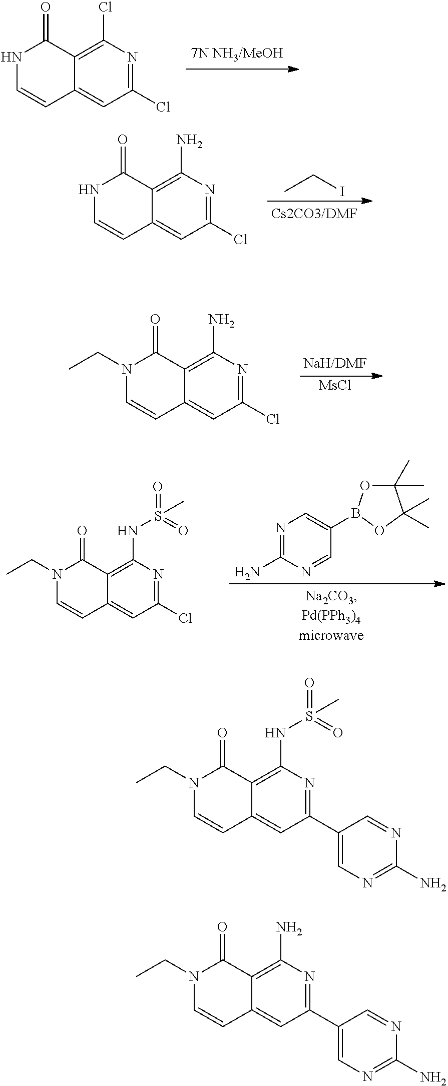

A suspension of 6,8-dichloro-2,7-naphthyridin-1(2H)-one (203 mg, 0.94 mmol) in ammonia solution in methanol (7N, 4 mL) was irradiated under microwave at 130° C. for 30 minutes. The reaction was diluted with water (4 mL). The solid was collected by filtration, washed with water and dried. The crude was used without further purification. ¹H NMR (CD₃OD) δ 7.26 (d, J=6.8 Hz, 1H), 6.30 (s, 1H), 6.35 (d, J=6.8 Hz, 1H); ESI-MS m/z 196.0 (MH⁺).

8-Amino-6-chloro-2,7-naphthyridin-1(2H)-one (140 mg, 0.72 mmol) was stirred with Cs₂CO₃ (440 mg, 1.35 mmol) in DMF (3 mL) at room temperature for 5 minutes. Iodoethane (147 mg, 0.945 mmol) in DMF (0.5 mL) was added. After stirring at room temperature for 20 minutes, the reaction was quenched with water (5 mL). The precipitate was collected by filtration, washed with water and dried to give 8-amino-6-chloro-2-ethyl-2,7-naphthyridin-1(2H)-one as a light yellow solid. ¹H NMR (CDCl₃) δ 9.02 (br s, 1H), 7.19 (d, J=7.2 Hz, 1H), 6.57 (s, 1H), 6.25 (d, J=7.2 Hz, 1H), 5.60 (br s, 1H), 4.00 (q, J=7.2 Hz, 2H), 1.38 (t, J=7.2 Hz, 3H); ESI-MS m/z 224.0 (MH⁺).

A mixture of 8-amino-6-chloro-2-ethyl-2,7-naphthyridin-1(2H)-one (46 mg, 0.21 mmol) and NaH (10 mg, 0.25 mmol, 60% in mineral oil) in DMF (1 mL) was stirred at room temperature. After 3 minutes, Ms₂O (44 mg, 0.25 mmol) was added. The reaction was stirred at room temperature for 2 hours. Additional NaH (10 mg, 0.25 mmol, 60% in mineral oil) was added to the reaction. After stirring at room temperature for 10 minutes, Ms₂O (40 mg) was added. The reaction continued overnight. The reaction was the quenched with water (4 mL), basified with saturated NaHCO₃. The precipitate was collected by filtration, washed with water and dried. The crude N-(3-chloro-7-ethyl-8-oxo-7,8-dihydro-2,7-naphthyridin-1-yl)methanesulfonamide was used without purification. ESI-MS m/z 302.0 (MH⁺).

A mixture of N-(3-chloro-7-ethyl-8-oxo-7,8-dihydro-2,7-naphthyridin-1-yl)methanesulfonamide (15 mg, 0.05 mmol), 5-(4,4,5,5-tetramethyl-1,3,2-dioxaborolan-2-yl)pyrimidin-2-amine (22 mg, 0.1 mmol), Pd(PPh₃)₄(14 mg, 0.012 mmol) and Na₂CO₃(16 mg, 0.15 mmol) in dioxane (1 mL) and water (0.2 mL) was degassed by a stream of argon. The mixture was irradiated in a sealed vial under microwave for 15 minutes at 170° C. The reaction mixture was purified by HPLC. Two fractions were collected. The first fraction was obtained as N-(3-(2-Aminopyrimidin-5-yl)-7-ethyl-8-oxo-7,8-dihydro-2,7-naphthyridin-1-yl)methanesulfonamide. ¹H NMR (400 MHz, CD₃OD) δ 9.09 (s, 2H), 6.38 (d, J=7.2 Hz, 1H), 7.60 (s, 1H), 6.70 (d, J=7.2 Hz, 1H), 4.08 (q, J=7.6 Hz, 2H), 3.50 (s, 3H), 1.37 (t, J=7.2 Hz, 3H); ESI-MS m/z 361.0 (MH⁺). A second fraction was obtained as 8-amino-6-(2-aminopyrimidin-5-yl)-2-ethyl-2,7-naphthyridin-1(2H)-one (compound 743). ¹H NMR (CD₃OD) δ 8.73 (s, 2H), 7.93 (d, J=7.2 Hz, 1H), 7.15 (s, 1H), 6.67 (d, J=7.2 Hz, 1H), 4.11 (q, J=7.2 Hz, 2H), 1.38 (t, J=7.2 Hz, 3H); ESI-MS m/z 283.1 (MH⁺).

### Example 33 (Compound No. 773)

### Preparation of 6-(2-Aminopyrimidin-5-yl)-2-((3-methyloxetan-3-yl)methyl)-8-(oxetan-3-ylamino)-2,7-naphthyridin-1(2H)-one

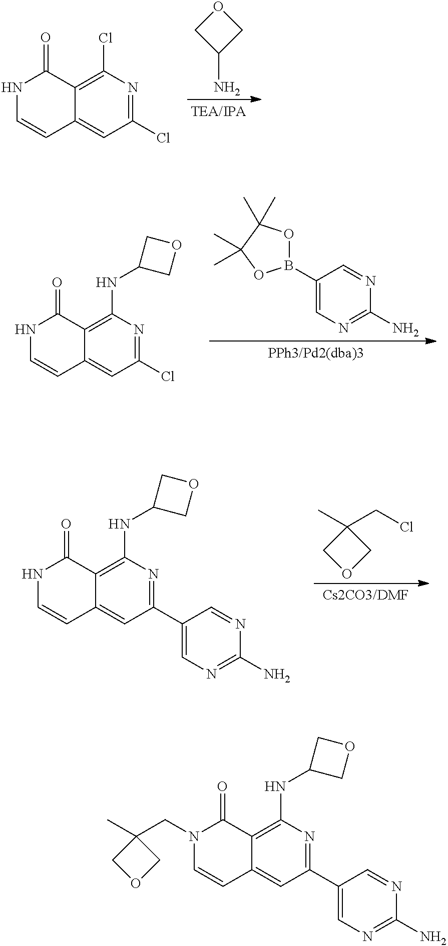

A mixture of 6,8-dichloro-2,7-naphthyridin-1(2H)-one (1.00 g, 4.65 mmol), oxetan-3-amine (0.51 g, 7.0 mmol) and triethylamine (1.28 mL, 9.2 mmol) in anhydrous 2-propanol (7 mL) was stirred at 110° C. for 1 hour. The reaction was diluted with water (7 mL). The solid 6-chloro-8-(oxetan-3-ylamino)-2,7-naphthyridin-1(2H)-one was collected by filtration, washed with water and dried. ¹H NMR (DMSO-d₆) δ 11.73 (s, 1H), 9.95 (d, J=6.0 Hz, 1H), 7.39 (d, J=6.8 Hz, 1H), 6.76 (s, 1H), 6.40 (d, J=6.8 Hz, 1H), 5.00 (m, 1H), 4.86 (t, J=6.8 Hz, 2H), 4.47 (t, J=6.8 Hz, 2H); ESI-MS m/z 252.1 (MH⁺).

To a mixture of 6-chloro-8-(oxetan-3-ylamino)-2,7-naphthyridin-1(2H)-one (200 mg, 0.79 mmol), 5-(4,4,5,5-tetramethyl-1,3,2-dioxaborolan-2-yl)pyrimidin-2-amine (263 mg, 1.19 mmol), Na₂CO₃ (167 mg, 1.58 mmol) in dioxane (3 mL) and water (0.6 mL), was added Pd(PPh₃)₄ (91 mg, 0.079 mmol). The mixture was degassed, sealed and stirred at 100° C. for 3.5 hours. Water (3 mL) was added. The precipitate was collected by filtration, washed with water and dried. The crude was triturated with EtOAc (20 mL) to give 6-(2-aminopyrimidin-5-yl)-8-(oxetan-3-ylamino)-2,7-naphthyridin-1(2H)-one as a yellow solid. ESI-MS m/z 310.9 (MH⁺).

A mixture of above 6-(2-aminopyrimidin-5-yl)-8-(oxetan-3-ylamino)-2,7-naphthyridin-1(2H)-one (62 mg, 0.2 mmol), Cs₂CO₃ (130 mg, 0.4 mmol), sodium iodide (50 mg) and 3-(chloromethyl)-3-methyloxetane (48 mg, 0.4 mmol, 2 eq) in DMF (1 mL) was stirred at 50° C. for 1.5 hours. The reaction was diluted with water (15 mL), extracted with dichloromethane (3×30 mL), dichloromethane was washed with brine (10 mL), dried over Na₂SO₄ and evaporated. The residue was purified by flash chromatography (MeOH: dichloromethane/0-10%) to give 6-(2-aminopyrimidin-5-yl)-2-((3-methyloxetan-3-yl)methyl)-8-(oxetan-3-ylamino)-2,7-naphthyridin-1(2H)-one as a white solid. ¹H NMR (CDOD) δ 8.94 (s, 1H), 7.88 (s, 1H), 7.43 (d, J=4.8 Hz, 1H), 7.01 (s, 1H), 6.49 (d, J=4.8 Hz, 1H), 5.25 (m, 1H), 5.07 (t, J=5.2 Hz, 2H), 4.81 (d, J=4.4 Hz, 2H), 4.70 (m, 2H), 4.33 (d, J=4.4 Hz, 2H), 4.18 (s, 2H), 1.37 (s, 3H); ESI-MS m/z 394.8 (MH⁺).

### Example 34 (Compound No. 779)

### Synthesis of 2-ethyl-6-(pyrazin-2-ylamino)-8-((3,3,3-trifluoro-2-hydroxypropyl)amino)-2,7-naphthyridin-1(2H)-one

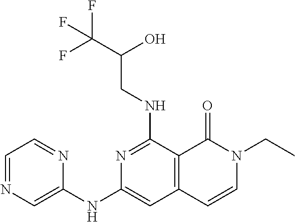

A mixture of 6-chloro-2-ethyl-8-((3,3,3-trifluoro-2-hydroxypropyl)amino)-2,7-naphthyridin-1(2H)-one (1.0 equiv), 4-methylpyridin-2-amine (2.0 equiv), Pd₂(DBA)₃ (5 mol %), Xantphos (20 mol %), Cs₂CO₃ (3.0 equiv) in tert-butanol was heated in microwave vial at 150° C. with oil bath. After 3 hours, the reaction mixture was diluted with DMSO and purified by prep HPLC. The compound was isolated as a TFA salt. ESI-MS m/z 395.20 (MH⁺), ¹H NMR (MeOD-d₄) δ 8.56 (s, 1H), 8.24 (m, 2H), 7.58 (d, J=7.2 Hz, 1H), 6.45 (s, 1H), 6.42 (d, T=7.2 Hz, 1H), 4.42 (m, 1H), 4.02 (m, 1H), 3.99 (q, T=6.8 Hz, 2H), 3.79 (m, 1H), 1.33 (t, J=6.8 Hz, 3H).

### Example 35 (Compound No. 789)

### Synthesis of 2-ethyl-8-((1S,3S)-3-(hydroxymethyl)cyclopentyl)amino)-6-((4-methylpyridin-2-yl)amino)-2,7-naphthyridin-1(2H)-one

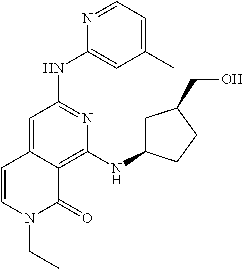

The mixture of 6-chloro-2-ethyl-8-(((1S,35)-3-(hydroxymethyl)cyclopentyl)amino)-2,7-naphthyridin-1(2H)-one (1.0 equiv), 4-methylpyridin-2-amine (2.0 equiv), Pd₂(DBA)₃ (5 mol %), Xantphos (20 mol %), Cs₂CO₃ (3.0 equiv) in tert-butanol was heated in microwave vial at 150° C. with oil bath. After 3 hours, the reaction mixture was diluted with DMSO and purified by prep HPLC. The compound was isolated as a TFA salt. ESI-MS m/z 394.30 (MH⁺), ¹H NMR (MeOD-d₄) δ 8.22 (d, J=6.0 Hz, 1H), 7.52 (d, J=7.2 Hz, 1H), 7.10 (d, J=6.0 Hz, 1H), 7.05 (s, 1H), 6.40 (d, J=7.2 Hz, 1H), 6.14 (s, 1H), 4.33 (m, 1H), 4.01 (q, J=7.2 Hz, 2H), 3.59 (m, 2H), 2.48 (s, 3H), 2.43 (m, 1H), 2.33 (m, 1H), 2.03 (m, 1H), 1.98 (m, 2H), 1.79 (m, 1H), 1.60 (m, 1H), 1.34 (t, J=7.2 Hz, 3H).

### Example 36 (Compound No. 855)

### 8-(tert-butylamino)-2-(2-hydroxyethyl)-6-((2′-methyl-[4,4′-bipyridin]-2-yl)amino)-2,7-naphthyridin-1(2H)-one

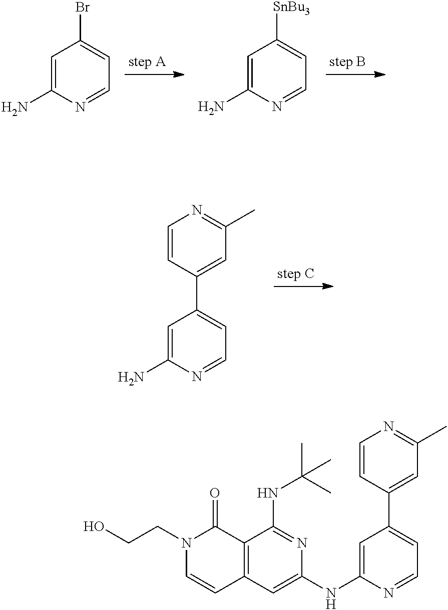

Step A: To a mixture of 4-bromopyridin-2-amine (340 mg, 1.95 mmol) in toluene (6 mL) was added hexabutyldistannane (1.25 g, 2.15 mmol) and Pd(PPh₃)₄ (45 mg, 0.039 mmol). The resulted mixture was degassed and heated to 105° C. under N₂ for 72 hours. After cooling to room temperature, the mixture was first treated with saturated aqueous KF solution (10 mL) and then extracted with EtOAc (3×25 mL). The organic layers were combined and treated with brine and dried over MgSO₄. After removing the drying agent by filtration, the filtrate was concentrated and purified by flash column chromatography (0-80% EtOAc/hexane) to provide 4-(tributylstannyl)pyridin-2-amine as a colorless oil.

Step B: To a solution of 4-(tributylstannyl)pyridin-2-amine (50 mg, 0.13 mmol) in toluene (1 mL) was added 4-bromo-2-methylpyridine (27 mg, 0.16 mmol) and Pd(PPh₃)₄ (14 mg, 0.012 mmol). The resulted mixture was degassed and heated to 105° C. under N₂ for 16 hours. After cooling to room temperature, the mixture was first treated with saturated aqueous KF solution (1 mL) and then extracted with EtOAc (3×3 mL). The organic layers were combined and concentrated. The resulted residue was purified by flash column chromatography (0˜10% MeOH/DCM) to provide 2′-methyl-[4,4′-bipyridin]-2-amine as a colorless solid.

Step C, 1.0 mL tert-butanol was added to the mixture of -(tert-butylamino)-7-chloro-3-(2-hydroxyethyl)pyrido[4,3-d]pyrimidin-4(3H)-one (10.0 mg, 0.034 mmol), 2′-methyl-[4,4′-bipyridin]-2-amine (15 mg, 0.08 mmol), Pd₂(dba)₃ (3 mg, 10%), Xantphos (4 mg, 20%), Cs₂CO₃ (40 mg, 0.12 mmol). The mixture was heated in a microwave reactor at 160° C. for 30 minutes. The mixture was then treated with saturated NH₄Cl aqueous solution (3 mL) and extracted with EtOAc (3×3 mL). The combined organic layer was concentrated and purified by preparative LC/MS to provide 8-(tert-butylamino)-2-(2-hydroxyethyl)-6-((2′-methyl-[4,4′-bipyridin]-2-yl)amino)-2,7-naphthyridin-1(2H)-one. ESI-MS m/z 445.2 (MH⁺).

### Example 37 (Compound No. 868)

### 5-(tert-butylamino)-7-((5-fluoro-4-(1-hydroxyethyl)pyridin-2-yl)amino)-3-(2-hydroxyethyl)pyrido[4,3-d]pyrimidin-4(3H)-one

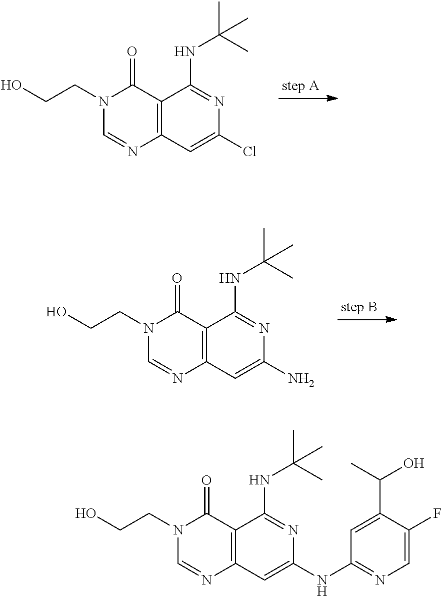

Step A: To a solution of 5-(tert-butylamino)-7-chloro-3-(2-hydroxyethyl)pyrido[4,3-d]pyrimidin-4(3H)-one (150 mg, 0.51 mmol) in anhydrous dioxane (2 mL) was added Pd₂(dba)₃ (23 mg, 5%) and (2-biphenyl)dicyclohexyl-phosphine (18 mg, 10%). The reaction mixture was then degassed and LHMDS (1.52 mL, 1.52 mmol, 1.0 N in THF) added. After the addition, the mixture was heated to 65° C. under N₂ for 14 hours and then cooled to room temperature. The mixture was treated with saturated NH₄Cl aqueous solution (5 mL) and extracted with EtOAc (3×10 mL). The combined organic layer was concentrated and purified by flash column chromatography (0-5% MeOH/DCM) to provide 7-amino-5-(tert-butylamino)-3-(2-hydroxyethyl)pyrido[4,3-d]pyrimidin-4(3H)-one as a light yellow solid.

Step B: To a solution of 7-amino-5-(tert-butylamino)-3-(2-hydroxyethyl)pyrido[4,3-d]pyrimidin-4(3H)-one (20 mg, 0.072 mmol) in anhydrous THF (1 mL) was added Pd₂(dba)₃ (7 mg, 10%) and BINAP (7 mg, 20%), NaOtBu (14 mg, 0.14 mmol) and 1-(2-bromo-5-fluoropyridin-4-yl)ethanol (17 mg, 0.076 mmol. Prepared as reported in Tetrahetron Letter, 2009, 50, 383-385). The reaction mixture was then degassed and heated to 70° C. under N₂ for 3 hours. After cooling to room temperature, the mixture was treated with saturated NH₄Cl aqueous solution (2 mL) and extracted with EtOAc (3×3 mL). The combined organic layer was concentrated and purified by preparative LC/MS to provide 5-(tert-butylamino)-7-((5-fluoro-4-(1-hydroxyethyl)pyridin-2-yl)amino)-3-(2-hydroxyethyl)pyrido[4,3-d]pyrimidin-4(3H)-one as a light yellow solid. ESI-MS m/z 416.2 (MH⁺).

### Example 38 (Compound No. 871)

### Preparation of 8-(tert-butylamino)-6-((4-(1-hydroxycyclobutyl)pyridin-2-yl)amino)-2-(2-hydroxyethyl)-2,7-naphthyridin-1(2H)-one

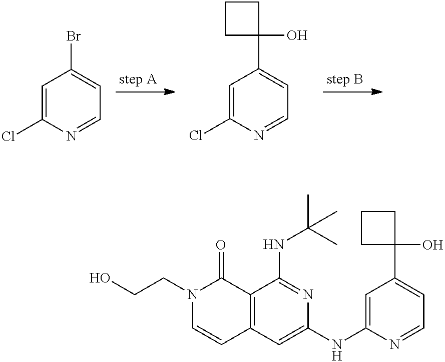

Step A: To a solution of 4-bromo-2-chloropyridine (193 mg, 1 mmol) in anhydrous THF (3 mL) at −78° C. was added BuLi (1.3 mmol, 0.52 mL 2.5 M in hexane) through syringe. After the addition, the mixture was stirred at −78° C. for 2 hours before the addition of cyclobutanone (105 mg, 1.5 mmol) dropwise through syringe. After the addition, the reaction mixture was slowly warmed up to room temperature and stirred for 4 hours. The reaction mixture was then poured into saturated NH₄Cl solution (20 mL) and extracted with EtOAc (3×30 mL). The combined organic layers was concentrated and purified by flash column chromatography (0˜60% EtOAc/hexane) to provide 1-(2-chloropyridin-4-yl)cyclobutanol as a white solid.

Step B: The titled compound 8-(tert-butylamino)-6-((4-(1-hydroxycyclobutyl)pyridin-2-yl)amino)-2-(2-hydroxyethyl)-2,7-naphthyridin-1(2H)-one was obtain from 7-amino-5-(tert-butylamino)-3-(2-hydroxyethyl)pyrido[4,3-d]pyrimidin-4(3H)-one and 1-(2-chloropyridin-4-yl)cyclobutanol as described in example 868 step B. ¹H NMR (CDCl₃) [symbol] 9.586 (s, 1H), 8.27 (d, J=5.2 Hz, 1H), 7.7.42 (s, 1H), 7.01 (d, J=7.2 Hz, 1H), 7.18 (b, 1H), 7.00 (m, 2H), 6.79 (s, 1H), 6.22 (d, J=7.2 Hz, 1H), 4.04 (m, 2H), 3.95 (m, 2H), 2.54 (m, 2H), 2.38 (m, 2H), 2.05-2.15 (m, 1H), 1.70-1.85 (m, 1H), 1.56 (s, 9H). ESI-MS m/z 424.2 (MH⁺)

### Example 39

### Compound 900

### Preparation of 6-(2-Aminopyrimidin-5-yl)-8-(tert-butylamino)-2-(2-hydroxyethyl)-2,7-naphthyridin-1(2H)-one

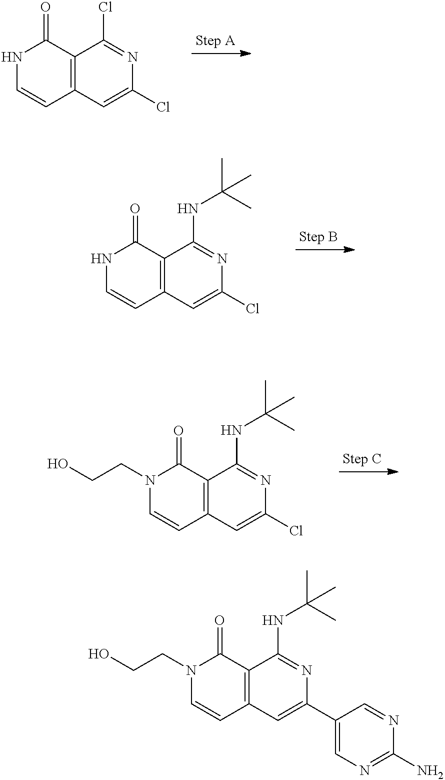

Step A: A mixture of 6,8-dichloro-2,7-naphthyridin-1(2H)-one (2.15 g, 10 mmol), tert-butylamine (1.2 mL, 12 mmol), Hunig's base (2.1 mL, 12 mmol) and 2-propanol (13 mL) is microwaved at 170° C. for 2 hours. The reaction mixture is cooled down to room temperature and worked-up to afford the crude 8-(tert-butylamino)-6-chloro-2,7-naphthyridin-1(2H)-one as a slightly yellow solid. 8-(tert-Butylamino)-6-chloro-2,7-naphthyridin-1(2H)-one is then used in step B without further purification.

Step B: A mixture of 8-(tert-butylamino)-6-chloro-2,7-naphthyridin-1(2H)-one (1.8 g, 7.14 mmol), 2-bromoethanol (0.77 mL, 10.8 mmol), Cs₂CO₃ (3.51 g, 10.8 mmol), DMF (25 mL) and NaI (135 mg) is stirred at 60° C. for 24 hours. The reaction is cooled down to room temperature and the reaction mixture is poured into ice water. The resulting precipitates are collected by vacuum filtration, washed with water and dried to get the crude 8-(tert-butylamino)-6-chloro-2-(2-hydroxyethyl)-2,7-naphthyridin-1(2H)-one as sight yellow solid.

Step C: A mixture of 8-(tert-butylamino)-6-chloro-2-(2-hydroxyethyl)-2,7-naphthyridin-1(2H)-one (1.95 g, 6.59 mmol), 5-(4,4,5,5-tetramethyl-1,3,2-dioxaborolan-2-yl)pyrimidin-2-amine (1.61 g, 7.25 mmol), Pd(PPh₃)₄ (305 mg, 0.264 mmol), K₂CO₃ (2.75 g, 19.77 mmol), 2-propanol (54 mL) and H₂O (18 mL) is stirred at 100° C. for overnight. The reaction mixture is cooled down to room temperature and worked-up. The residue is purified on slilica gel flash column chromatography (eluent: 0-10% methanol in dichloromethane) to afford 6-(2-aminopyrimidin-5-yl)-8-(tert-butylamino)-2-(2-hydroxyethyl)-2,7-naphthyridin-1(2H)-one as a white solid. ¹H NMR (DMSO-d₆) δ 9.68 (s, 1H), 8.93 (s, 2H), 7.49 (d, J=7.2 Hz, 1H), 7.07 (s, 1H), 7.03 (s, 2H), 6.36 (d, J=7.2 Hz, 1H), 4.88 (t, J=5.6 Hz, 1H), 3.95 (m, 2H), 3.64 (m, 2H), 1.52 (s, 9H). ESI-MS m/z 355.10 (MH⁺).

### Example 40 (Compound 922) & Example 41 (Compound 923)

### Preparation of 6-(2-Aminopyrimidin-5-yl)-8-(tert-butylamino)-2-(oxetan-3-yl)-2,7-naphthyridin-1(2H)-one and 6-(2-Aminopyrimidin-5-yl)-8-(tert-butylamino)-2-(1,3-dihydroxypropan-2-yl)-2,7-naphthyridin-1(2H)-one

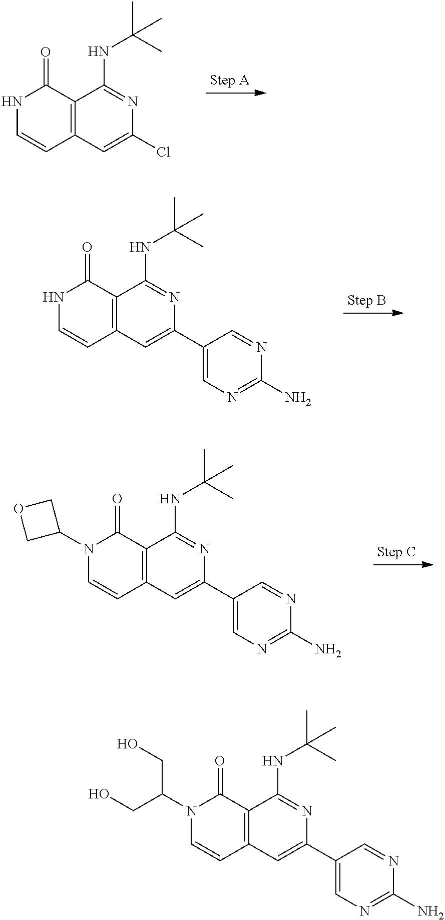

Step A: A mixture of 8-(tert-butylamino)-6-chloro-2,7-naphthyridin-1(2H)-one (252 mg, 1 mmol), 5-(4,4,5,5-tetramethyl-1,3,2-dioxaborolan-2-yl)pyrimidin-2-amine (244 mg, 1.1 mmol), Pd(PPh₃)₄ (46 mg, 0.04 mmol), K₂CO₃ (414 mg, 3 mmol), 2-propanol (9 mL) and H₂O (3 mL) is stirred at 100° C. for overnight. The reaction mixture is cooled down to room temperature and worked-up. The residue is purified on slilica gel flash column chromatography (eluent: 0-10% methanol in dichloromethane) to afford 6-(2-aminopyrimidin-5-yl)-8-(tert-butylamino)-2,7-naphthyridin-1(2H)-one as a slightly yellow solid. ¹H NMR (DMSO-d₆) δ 11.33 (s, 1H), 9.59 (s, 1H), 8.92 (s, 2H), 7.25 (d, J=6.8 Hz, 1H), 7.07 (s, 1H), 7.03 (s, 2H), 6.32 (d, J=6.8 Hz, 1H), 1.51 (s, 9H). ESI-MS m/z 311.10 (MH⁺).

Step B: A mixture of 6-(2-aminopyrimidin-5-yl)-8-(tert-butylamino)-2,7-naphthyridin-1(2H)-one (70 mg, 0.226 mmol), 3-iodooxetane (84 mg, 0.452 mmol), Cs₂CO₃ (148 mg, 0.452 mmol), DMF (4 mL) is stirred at 60° C. for overnight. The reaction mixture is cooled down to room temperature and worked-up. The residue is purified on slilica gel flash column chromatography (eluent: 0-10% methanol in dichloromethane) to afford 6-(2-aminopyrimidin-5-yl)-8-(tert-butylamino)-2-(oxetan-3-yl)-2,7-naphthyridin-1(2H)-one as a slightly yellow solid. ¹H NMR (DMSO-d₆) δ 9.50 (s, 1H), 8.93 (s, 2H), 7.68 (d, J=7.6 Hz, 1H), 7.12 (s, 1H), 7.06 (s, 2H), 6.49 (d, J=7.6 Hz, 1H), 5.52 (m, 1H), 4.87 (m, 2H), 4.77 (m, 2H), 1.51 (s, 9H). ESI-MS m/z 367.10 (MH⁺).

Step C: A mixture of 6-(2-aminopyrimidin-5-yl)-8-(tert-butylamino)-2-(oxetan-3-yl)-2,7-naphthyridin-1(2H)-one (45 mg, 0.123 mmol), LiOH (45 mg), 2-propanol (2 mL) and H₂O (2 mL) is stirred at 110° C. for overnight. The reaction mixture is cooled down to room temperature and worked-up. The residue is purified on slilica gel flash column chromatography (eluent: 0-10% methanol in dichloromethane) to afford 6-(2-aminopyrimidin-5-yl)-8-(tert-butylamino)-2-(1,3-dihydroxypropan-2-yl)-2,7-naphthyridin-1(2H)-one as a slightly yellow solid. ¹H NMR (DMSO-d₆) δ 9.76 (s, 1H), 8.93 (s, 2H), 7.54 (d, J=7.2 Hz, 1H), 7.06 (s, 1H), 7.03 (s, 2H), 6.38 (d, J=7.2 Hz, 1H), 4.94-4.88 (m, 3H), 3.76-3.64 (m, 4H), 1.53 (s, 9H). ESI-MS m/z 385.10 (MH⁺).

By repeating the procedures provided in the above examples, using appropriate starting materials, the following compounds of Formula I, in particular compounds of Formula (I) as identified in Table 1, were obtained.

TABLE 1

| | | Physical Data |
|---|---|---|
| | | ¹H NMR 400 MHz (CDCl₃), |
| | | HPLC Retention Time, and/or |
| Cmpd | | MS (m/z) |
| No. | Structure | Syk Enzyme IC₅₀ (uM) |
| 1 | 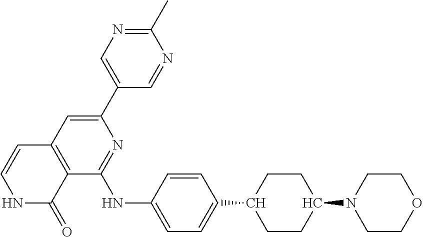 | MS m/z 497.2 (M + 1). IC₅₀ (uM): 0.049 |
| 2 | 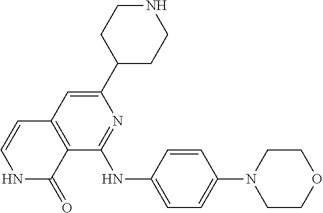 | MS m/z 406.4 (M + 1). IC₅₀ (uM): 0.249 |
| 3 | 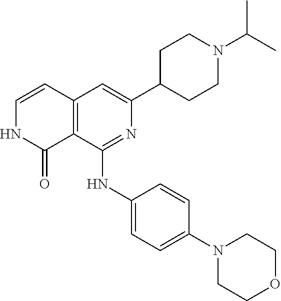 | MS m/z 448.2 (M + 1). IC₅₀ (uM): 0.202 |
| 4 | 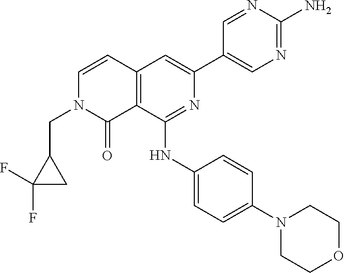 | MS m/z 506.1 (M + 1). IC₅₀ (uM): 0.489 |
| 5 | 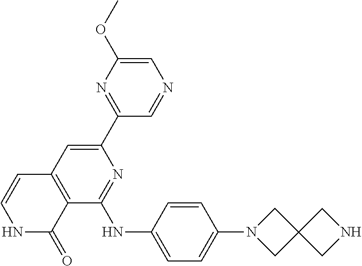 | MS m/z 442.4 (M + 1). IC₅₀ (uM): 0.316 |
| 6 | 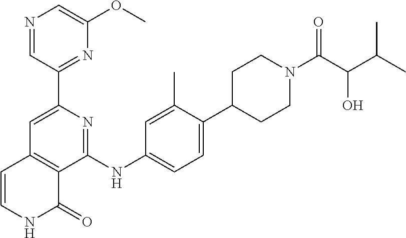 | ¹H NMR (400 MHz, DMSO- d6) δ 12.02 (s, 1H), 9.01 (s, 1H), 8.40 (s, 1H), 7.81 (s, 1H), 7.72 (dd, 1H), 7.65 (d, 1H), 7.48 (t, 1H), 7.24 (d, 1H), 6.63 (d, 1H), 4.09 (m, 1H), 4.07 (s, 3H), 3.59- 3.58 (m,2H), 3.30-3.27 (m, 2H), 3.14-3.06 (m, 2H), 2.82-2.76 (m, 1H), 2.72 (s, 2H), 2.14 (m, 2H), 1.97 (m, 2H), 0.84 (d, 3H), 0.80 (d, 3H); MS m/z 543.1(M + 1). IC₅₀ (uM): 0.398 |
| 7 | 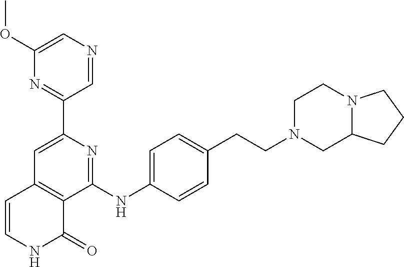 | ¹H NMR (400 MHz, DMSO- d6) δ 12.03 (s, 1H), 9.06 (s, 1H), 7.84 (d, 2H), 7.85 (s, 1H), 7.49 (t, 1H), 7.36 (d, 2H), 6.69 (d, 1H), 4.08 (s, 3H), 3.43- 3.40 (m, 2H), 3.32-3.31 (m, 1H), 3.09 (q, 2H), 2.50 (brs, 8H), 1.28 (m, 4H); MS m/z 498.4 (M + 1). IC₅₀ (uM): 0.053 |
| 8 | 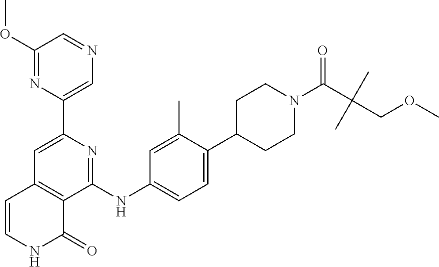 | ¹H NMR (400 MHz, DMSO- d6) δ 11.94 (s, 1H), 11.88 (brs, 1H), 9.07 (s, 1H), 8.40 (s, 1H), 7.81 (dd, 1H), 7.65 (s, 1H), 7.46 (t, 1H), 7.22 (d, 1H), 6.67 (d, 1H), 4.07 (s, 3H), 3.59- 3.58 (m, 2H), 3.30-3.27 (m, 3.06 (m, 2H), 2.82-2.76 (m, 1H), 2.54 (s, 2H), 2.39 (s, 3H) 2.14 (m, 2H), 1.22 (s, 6H); MS m/z 557.1 (M + 1). IC₅₀ (uM): 0.253 |
| 9 | 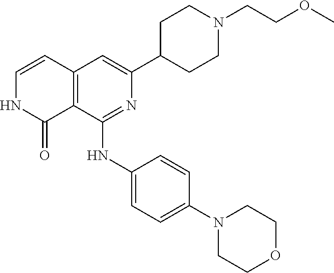 | MS m/z 464.3 (M + 1). IC₅₀ (uM): 0.299 |
| 10 | 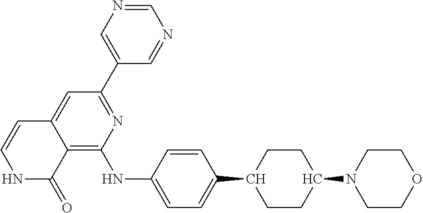 | MS m/z 483.1 (M + 1). IC50 (uM): 0.266 |
| 11 | 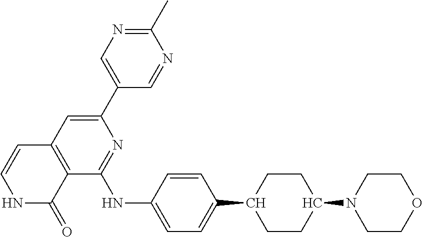 | MS m/z 497.4 (M + 1). IC50 (uM): 0.652 |
| 12 | 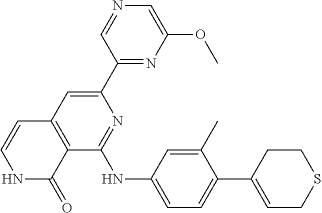 | MS m/z 458.2 (M + 1). IC50 (uM): 3.426 |
| 13 | 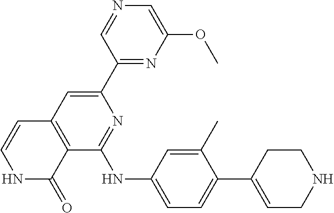 | MS m/z 441.1 (M + 1). IC50 (uM): 0.038 |
| 14 | 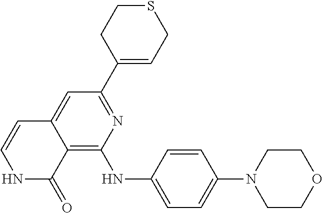 | MS m/z 421.5 (M + 1). IC50 (uM): 0.809 |
| 15 | 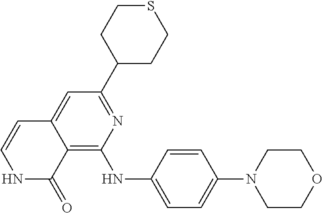 | MS m/z 423.2 (M + 1). IC50 (uM): 0.702 |
| 16 | 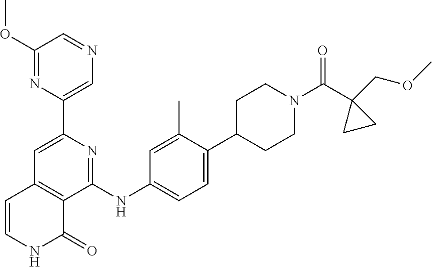 | MS m/z 555.2 (M + 1). IC50 (uM): 0.099 |
| 17 | 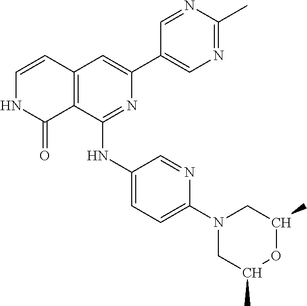 | MS m/z 444.3 (M + 1). IC50 (uM): 0.163 |
| 18 | 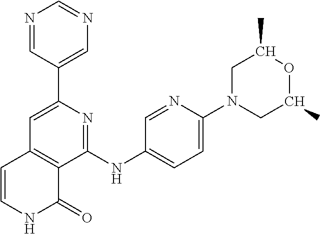 | MS m/z 430.2 (M + 1). IC50 (uM): 0.245 |
| 19 | 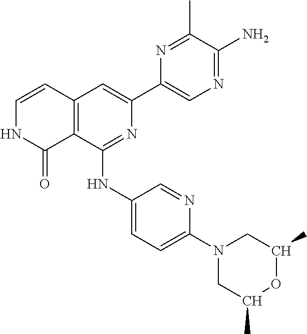 | MS m/z 459.1 (M + 1). IC50 (uM): 0.222 |
| 20 | 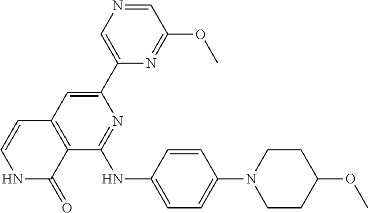 | MS m/z 459.2 (M + 1). IC50 (uM): 0.318 |
| 21 | 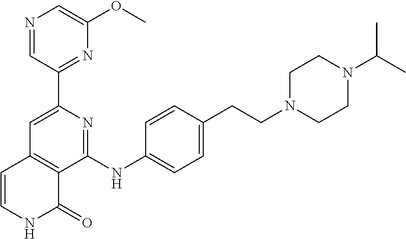 | MS m/z 500.6 (M + 1). IC50 (uM): 0.086 |
| 22 | 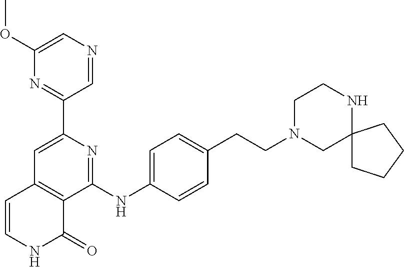 | MS m/z 512.1 (M + 1). IC50 (uM): 0.078 |
| 23 | 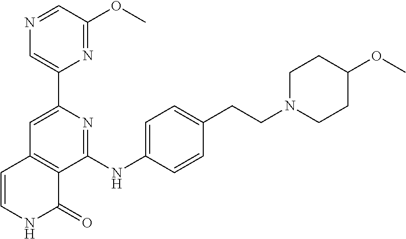 | MS m/z 487.2 (M + 1). IC50 (uM): 0.039 |
| 24 | 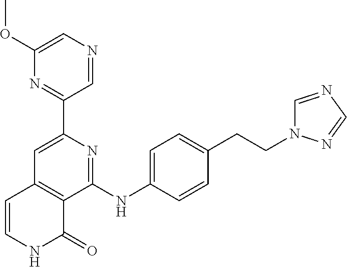 | ¹H NMR (400 MHz, DMSO- d6) δ 11.95 (s, 1H), 11.92 (brs, 1H), 9.45 (s, 1H), 7.80 (s, 1H), 7.77 (d, 2H), 7.46 (t, 1H), 7.29 (d, 2H), 6.66 (d, 1H), 4.48 (t, 2H), 3.12 (t, 2H); MS m/z 441.2 (M + 1). IC50 (uM): 0.122 |
| 25 | 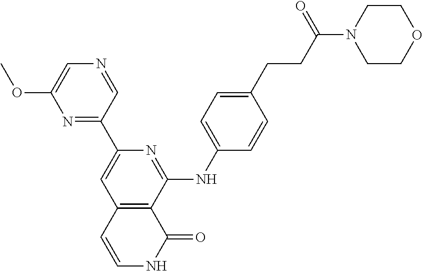 | ¹H NMR (400 MHz, DMSO- d6) δ 11.95 (s, 1H), 11.87 (brs, 1H), 9.04 (s, 1H), 7.80- 7.77 (m, 3H), 7.46 (t, 1H), 7.29 (d, 2H), 6.66 (d, 1H), 4.08 (s, 3H), 3.52-3.50 (m, 2H), 2.82 (t, 2H), 2.64 (t, 2H), 2.54 (brs, 6H) MS m/z 487.2 (M + 1). IC50 (uM): 0.024 |
| 26 | 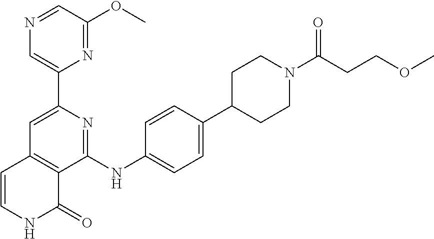 | ¹H NMR (400 MHz, DMSO- d6) δ 11.94 (s, 1H), 11.87 (brs, 1H), 9.01 (s, 1H), 8.40 (s, 1H), 7.79 (s, 1H), 7.74 (d, 1H), 7.64 (d, 1H), 7.45 (t, 1H), 7.22 (d, 1H), 6.64 (d, 1H), 4.03 (s, 3H), 3.92 (s, 3H), 3.72-3.30 (m, 2H), 3.59-3.58 (m, 2H), 3.35 (s, 3H), 3.30-3.27 (m, 2H), 3.14-3.06 (m, 2H), 2.82-2.76 (m, 1H), 2.72 (s, 3H), 2.14 (m, 2H); MS m/z 515.5 (M + 1). IC50(uM): 0.072 |
| 27 | 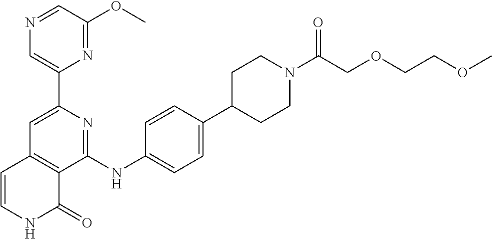 | MS m/z 545.4 (M + 1). IC50 (uM): 0.044 |
| 28 | 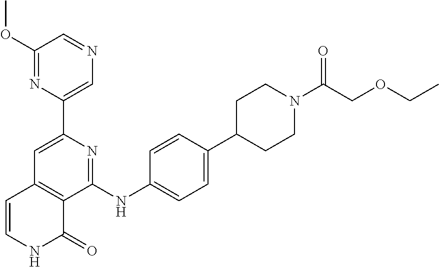 | MS m/z 515.1 (M + 1). IC50 (uM): 0.115 |
| 29 | 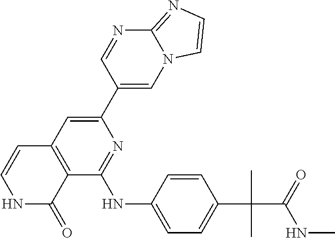 | MS m/z 454.2 (M + 1). IC50 (uM): 0.03 |
| 30 | 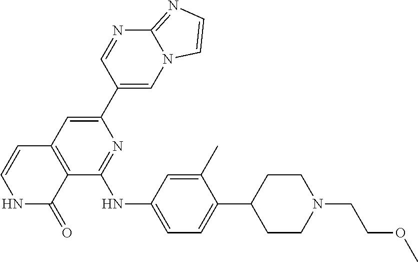 | MS m/z 510.2 (M + 1). IC50 (uM): 0.008 |
| 31 | 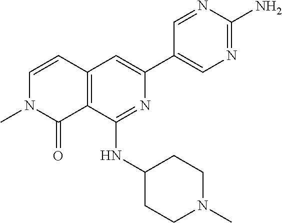 | MS m/z 366.1 (M + 1). IC50 (uM): 1.766 |
| 32 | 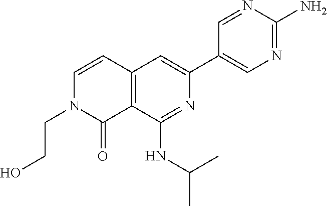 | MS m/z 341.3 (M + 1). IC50 (uM): 0.14 |
| 33 | 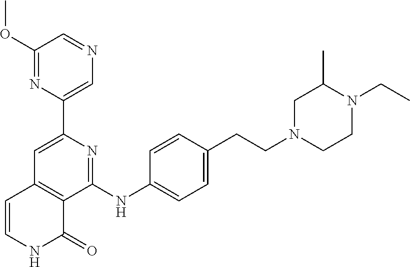 | MS m/z 500.6 (M + 1). IC50 (uM): 0.034 |
| 34 | 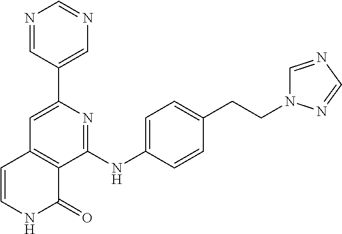 | MS m/z 411.2 (M + 1). IC50 (uM): 0.18 |
| 35 | 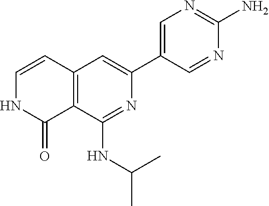 | MS m/z 297.3 (M + 1). IC50 (uM): 0.086 |
| 36 | 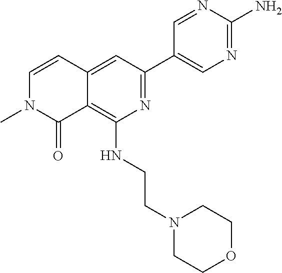 | MS m/z 382.1 (M + 1). IC50 (uM): 0.428 |
| 37 | 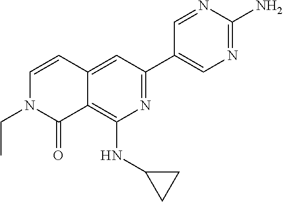 | MS m/z 323.1 (M + 1). IC50 (uM): 0.069 |
| 38 | 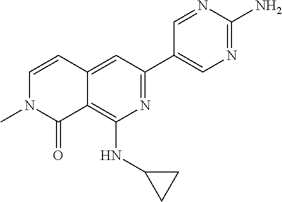 | MS m/z 309.2 (M + 1). IC50 (uM): 0.062 |
| 39 | 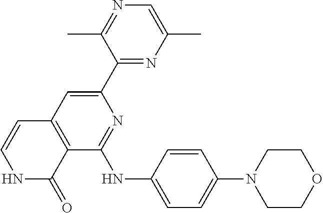 | MS m/z 482.2 (M + 1). IC50 (uM): 1.96 |
| 40 | 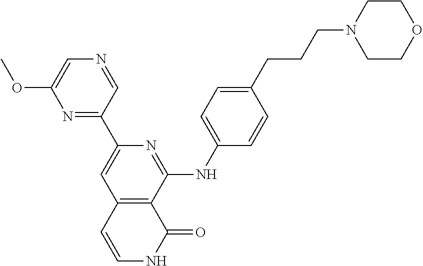 | MS m/z 473.4 (M + 1). IC50 (uM): 0.095 |
| 41 | 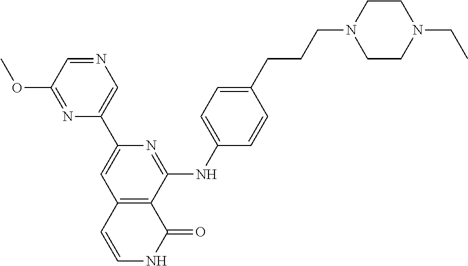 | ¹H NMR (400 MHz, DMSO- d6) δ 12.02 (s, 1H), 9.07 (s, 1H), 7.82 (d, 2H, J = 8.8 Hz), 7.85 (s, 1H), 7.47 (t, 1H, J = 6.4 Hz), 7.32 (d, 2H, J = 8.8 Hz), 6.66 (d, 1H, J = 6.8 Hz), 4.07 (s, 3H), 3.43-3.40 (m, 2H), 3.32-3.31 (m, 2H), 3.08 (q, 2H), 2.52 (brs, 10H), 1.29 (t, 3H); MS m/z 500.2 (M + 1). IC50 (uM): 0.042 |
| 42 | 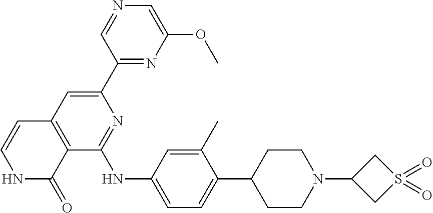 | MS m/z 547.1 (M + 1). IC50 (uM): 0.026 |
| 43 | 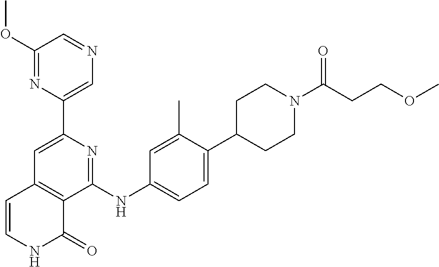 | MS m/z 429.2 (M + 1). IC50 (uM): 0.112 |
| 44 | 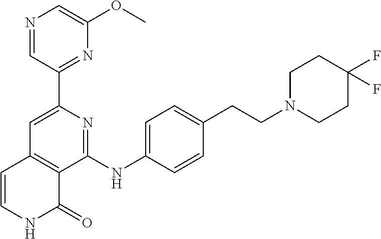 | MS m/z 493.5 (M + 1). IC50 (uM): 0.638 |
| 45 | 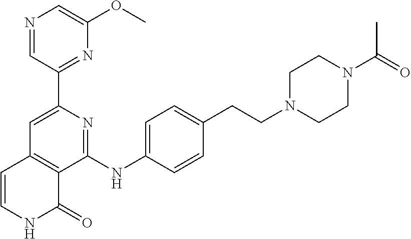 | MS m/z 500.3 (M + 1). IC50 (uM): 0.061 |
| 46 | 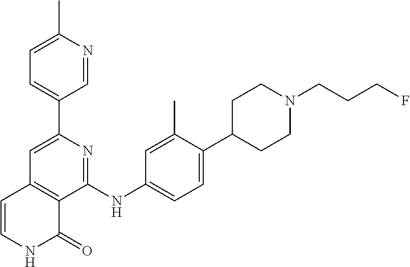 | MS m/z 486.1 (M + 1). IC50 (uM): 0.037 |
| 47 | 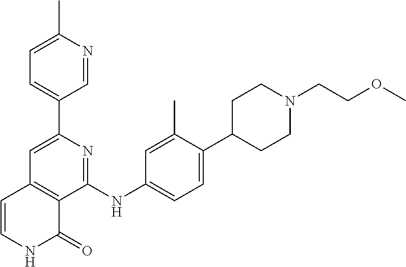 | MS m/z 484.3 (M + 1). IC50 (uM): 0.012 |
| 48 | 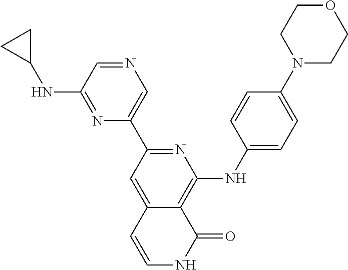 | MS m/z 456.2 (M + 1). IC50 (uM): 0.083 |
| 49 | 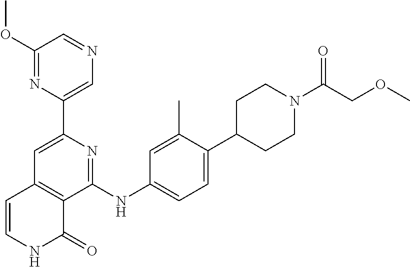 | ¹H NMR(400 MHz, DMSO- d6) δ 11.94 (s, 1H), 11.87 (brs, 1H), 9.01 (s, 1H),8.40 (s, 1H), 7.79 (s, 1H), 7.74 (d, 1H), 7.64 (d, 1H), 7.45 (t, 1H), 7.22 (d, 1H), 6.64 (d, 1H), 3.92 (s, 3H), 3.72- 3.30 (m, 2H), 3.59-3.58 (m, 2H), 3.35 (s, 3H), 3.30- 3.27 (m, 2H), 3.14-3.06 (m, 2H), 2.82- 2.76 (m, 1H), 2.72 (s, 3H), 214 (m, 2H), 1.97 (m, 2H); MS m/z 515.1 (M + 1). IC50 (uM): 0.075 |
| 50 | 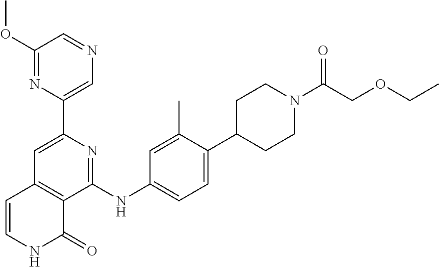 | MS m/z 529.2 (M + 1). IC50 (uM): 0.075 |
| 51 | 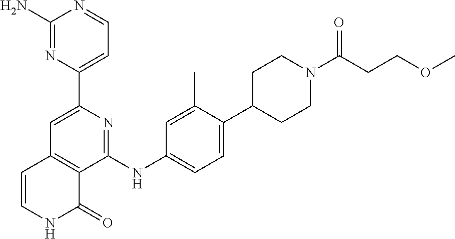 | MS m/z 528.6 (M + 1). IC50 (uM): 0.628 |
| 52 | 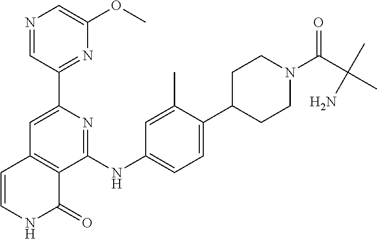 | MS m/z 528.6 (M + 1). IC50 (uM): 0.03 |
| 53 | 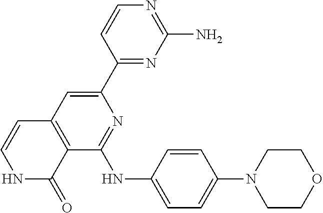 | MS m/z 416.4 (M + 1). IC50 (uM): 0.494 |
| 54 | 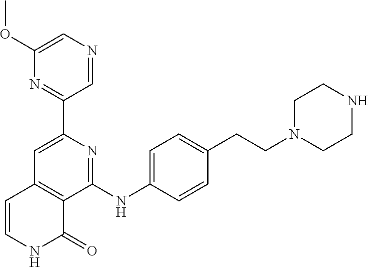 | MS m/z 458.1 (M + 1). IC50 (uM): 0.064 |
| 55 | 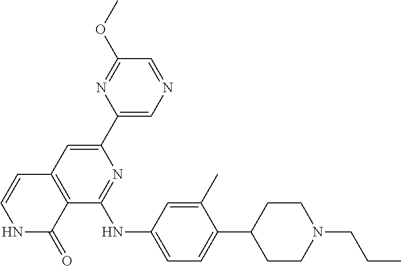 | MS m/z 485.2 (M + 1). IC50 (uM): 0.033 |
| 56 | 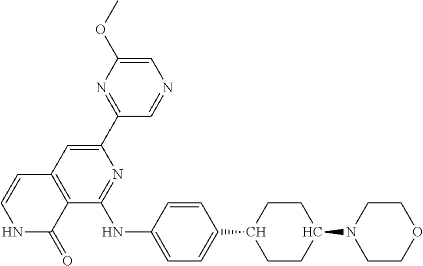 | MS m/z 513.1 (M + 1). IC50 (uM): 0.358 |
| 57 | 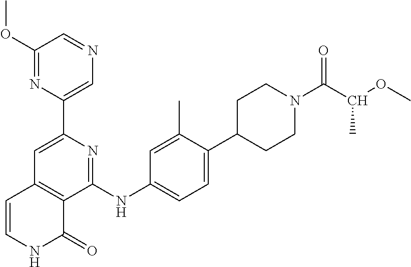 | MS m/z 529.2 (M + 1). IC50 (uM): 0.047 |
| 58 | 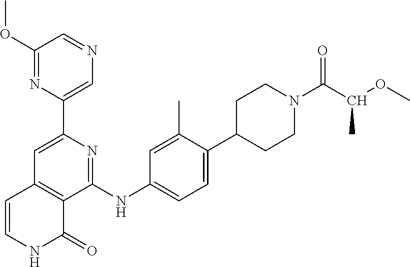 | ^{¹}H NMR (400 MHz DMSO- d6) δ 11.90 (s, 1H), 11.88 (s, 1H), 9.06 (s, 1H), 8.40 (s, 1H), 7.80 (s, 1H), 7.75 (brm, 1H), 7.64 (s, 1H), 7.46 (t, 1H), 7.31 (m, 1H), 7.23 (brs, 1H), 6.67 (d, 1H), 4.58-4.53 (m, 1H), 4.06 (s, 3H), 3.23 (s, 3H), 3.30- 3.27 (m, 2H), 3.14-3.06 (m, 2H), 2.82- 2.76 (m, 1H), 2.72 (s, 3H), 2.14 (m, 2H), 1.97 (m, 2H); 1.24 (d, 3H); MS m/z 529.2 (M + 1). IC50 (uM): 0.04 |
| 59 | 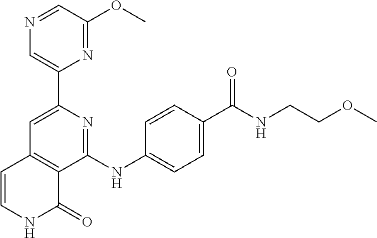 | MS m/z 447.3 (M + 1). IC50 (uM): 0.168 |
| 60 | 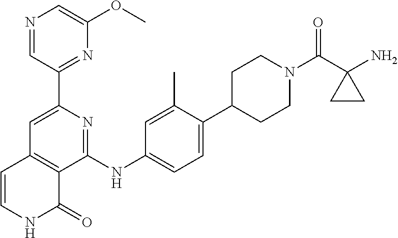 | ¹H NMR (400 MHz, DMSO- d6) δ 11.92 (s, 1H), 11.87 (s, 1H), 9.06 (s, 1H), H.42 (s, 1H), 7.92 (s, 1H), 7.74 (brm, 1H), 7.61 (s, 1H), 7.44 (t, 1H), 7.32 (m, 1H), 7.22 (brs, 1H), 6.65 (d, 1H), 4.33 (brs, 2H), 4.09 (s, 3H), 3.06-3.01 (m, 2H), 2.40 (s, 3H), 2.14 (m, 2H), 1.97 (m, 2H); 1.79- 1.63 (m, 3H), 1.25 (m, 4H); MS m/z 526.1 (M + 1). EC50 (uM): 0.155 |
| 61 | 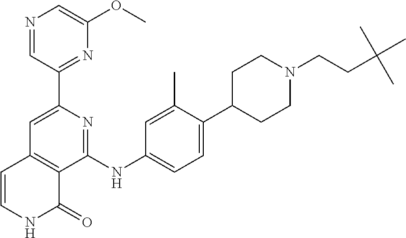 | MS m/z 527.2 (M + 1). IC50 (uM): 0.158 |
| 62 | 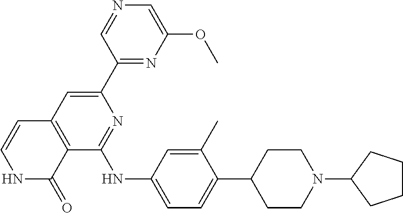 | MS m/z 511.5 (M + 1). IC50 (uM): 0.011 |
| 63 | 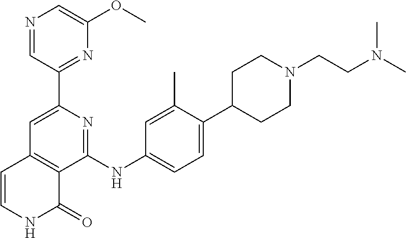 | MS m/z 514.3 (M + 1). IC50 (uM): 0.033 |
| 64 | 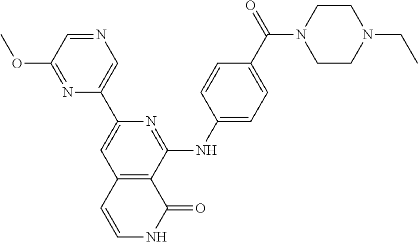 | ¹H NMR (400 MHz, DMSO- d6) δ 12.28 (s, 1H), 11.99 (s, 1H), 9.08 (s, 1H), 8.42 (s, 1H), 7.94 (d, 2H), 7.91 (s, 1H), 7.56 (d, 2H), 7.61 (s, 1H), 7.48 (t, 1H), 6.65 (d, 1H), 4.09 (s, 3H), 3.15- 3.13 (d, 2H), 2.50 (brm, 8H), 1.25 (t, 3H); MS m/z 486.2 (M + 1). IC50 (uM): 0.035 |
| 65 | 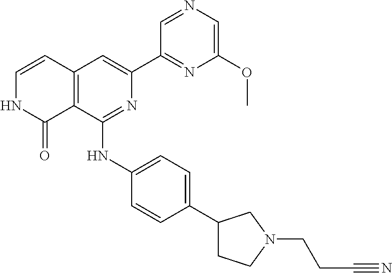 | MS m/z 468.1 (M + 1). IC50 (uM): 0.03 |
| 66 | 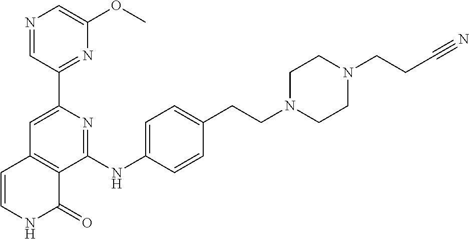 | MS m/z 511.2 (M + 1). IC50 (uM): 0.055 |
| 67 | 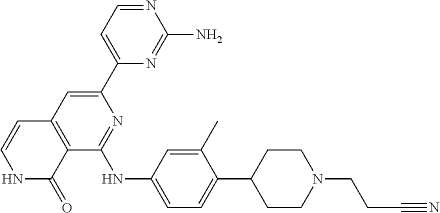 | MS m/z 481.3 (M + 1). IC50 (uM): 0.328 |
| 68 | 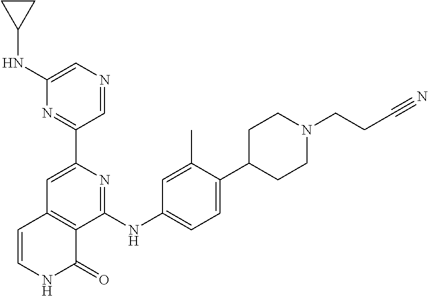 | MS m/z 521.4 (M + 1). IC50 (uM): 0.089 |
| 69 | 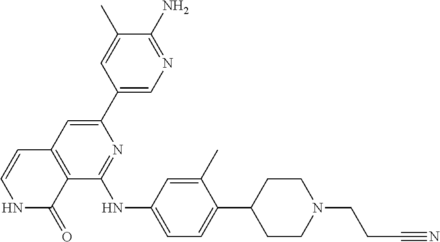 | MS m/z 494.1 (M + 1). IC50 (uM): 0.028 |
| 70 | 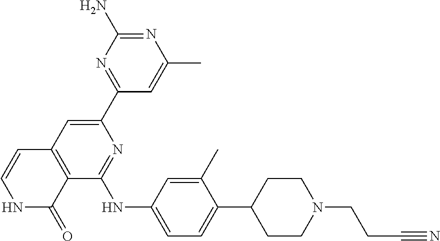 | MS m/z 495.1 (M + 1). IC50 (uM): 0.344 |
| 71 | 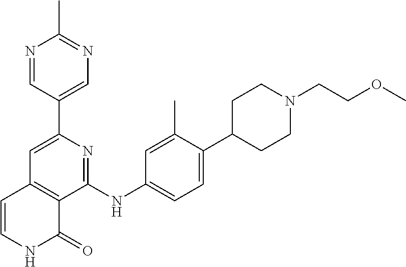 | MS m/z 485.1 (M + 1). IC50 (uM): 0.014 |
| 72 | 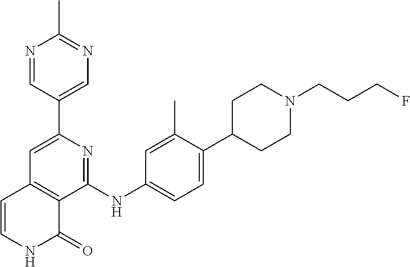 | MS m/z 487.2 (M + 1). IC50 (uM): 0.012 |
| 73 | 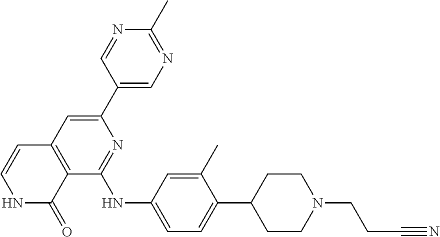 | MS m/z 480.2 (M + 1). IC50 (uM): 0.081 |
| 74 | 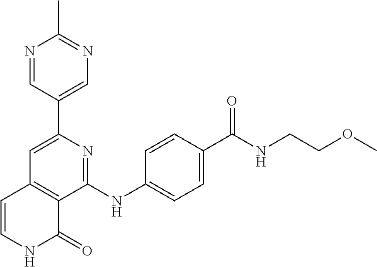 | MS m/z 431.2 (M + 1). IC50 (uM): 0.029 |
| 75 | 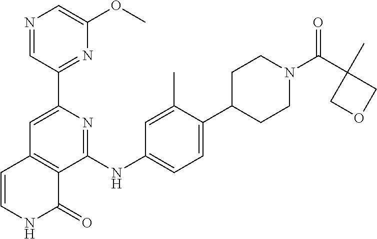 | MS m/z 541.2 (M + 1). IC50 (uM): 0.053 |
| 76 | 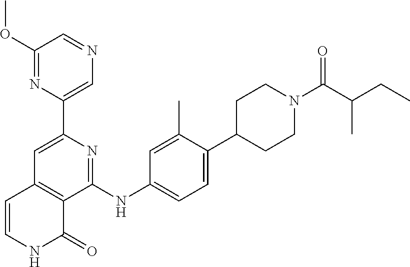 | MS m/z 527.2 (M + 1). IC50 (uM): 0.225 |
| 77 | 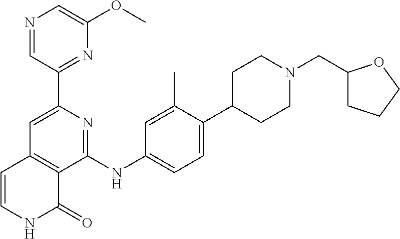 | MS m/z 527.2 (M + 1). IC50 (uM): 0.115 |
| 78 | 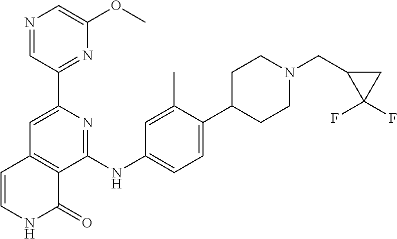 | MS m/z 533.1 (M + 1). IC50 (uM): 0.129 |
| 79 | 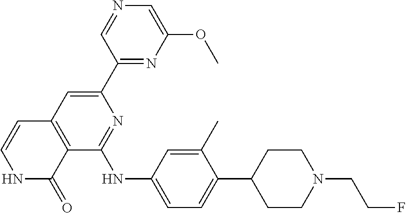 | MS m/z 489.3 (M + 1). IC50 (uM): 0.027 |
| 80 | 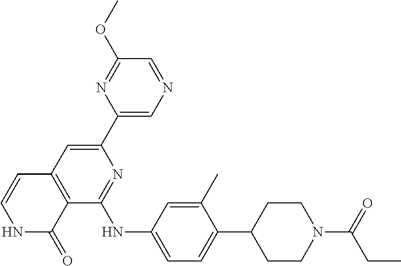 | MS m/z 499.1 (M + 1). IC50 (uM): 0.063 |
| 81 | 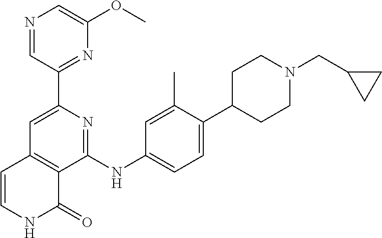 | MS m/z 497.3 (M + 1). IC50 (uM): 0.004 |
| 82 | 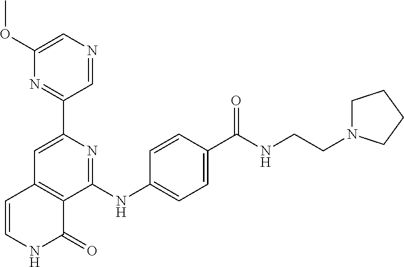 | MS m/z 486.3 (M + 1). IC50 (uM): 0.014 |
| 83 | 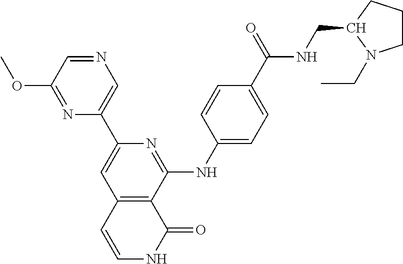 | MS m/z 500.1 (M + 1). IC50 (uM): 0.014 |
| 84 | 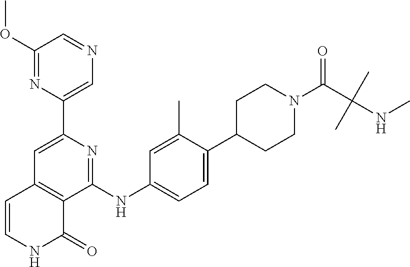 | MS m/z 542.3 (M + 1). IC50 (uM): 0.005 |
| 85 | 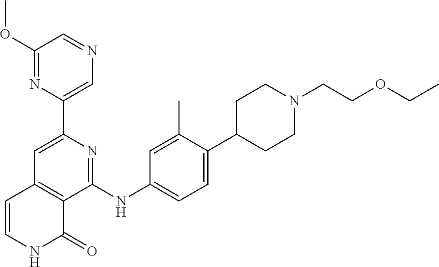 | MS m/z 515.3 (M + 1). IC50 (uM): 0.078 |
| 86 | 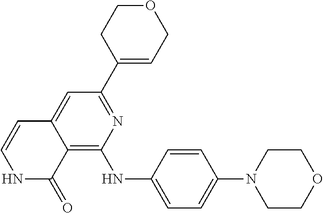 | MS m/z 405.2 (M + 1). IC50 (uM): 0.096 |
| 87 | 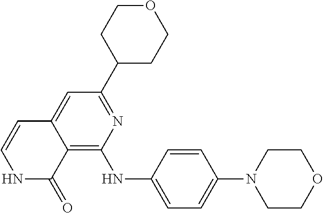 | MS m/z 407.1 (M + 1). IC50 (uM): 0.058 |
| 88 | 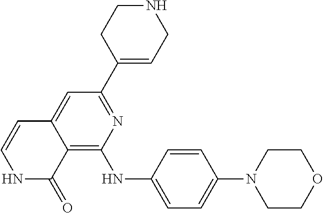 | MS m/z 404.2 (M + 1). IC50 (uM): 0.051 |
| 89 | 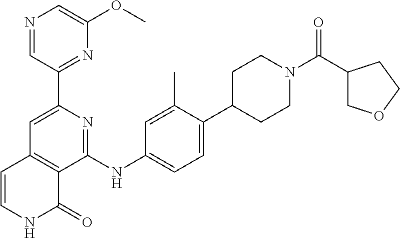 | MS m/z 541.3 (M + 1). IC50 (uM): 0.055 |
| 90 | 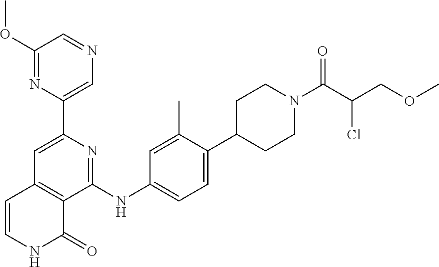 | MS m/z 564.3 (M + 1). IC50 (uM): 0.204 |
| 91 | 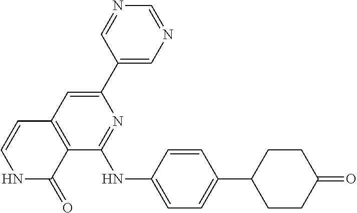 | MS m/z 412.1 (M + 1). IC50 (uM): 0.40 |
| 92 | 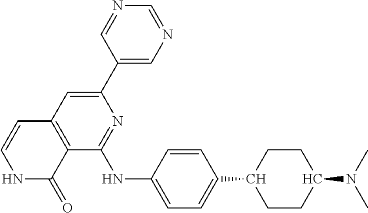 | MS m/z 441.2 (M + 1). IC50 (uM): 0.045 |
| 93 | 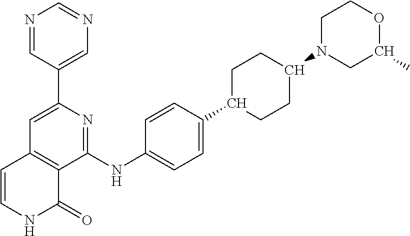 | ¹H NMR (400 MHz, DMSO- d6) δ 11.94 (s, 1H), 11.91 (brs, 1H), 9.42 (s, 2H), 9.21 (s, 1H), 7.75 (d, 2H), 7.69 (s, 1H), 7.45 (t, 1H), 7.23 (d, 2H), 6.53 1H), 3.91- 3.92 (m, 3H), 3.41 (m, 2H), 3.21-3.10 (m, 2H), 2.22 (d, 2H), 1.91 (d, 2H), 1.92 (d, 2H), 1.71-1.63 (m, 2H), 1.57-1.51 (m, 5H); MS m/z 497.4 (M + 1). IC50 (uM): 0.433 |
| 94 | 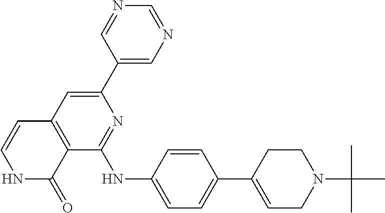 | MS m/z 453.2 (M + 1). IC50 (uM): 0.013 |
| 95 | 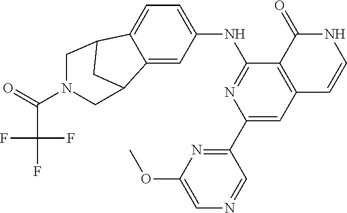 | ¹H NMR (400 MHz, DMSO- d6) δ 11.14 (s, 1H), 8.93 (s, 1H), 8.34 (s, 1H), 7.79 (s, 1H), 7.67 (s, 1H), 7.49 (dd, 1H), 7.42 (d, 1H), 7.38 (m, 1H), 6.57 (d, 1H), 4.38 (m, 1H), 4.0 (s, 3H), 3.92 (m, 1H), 3.57 (m, 1H), 3.41 (m. 2H), 3.20 (brs, 1H), 2.47 (m, 1H), 2.19 (d, 1H); MS m/z 523.2 (M + 1). IC50 (uM): 2.368 |
| 96 | 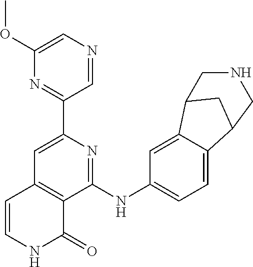 | ¹H NMR (400 MHz, DMSO- d6) δ 11.12 (s, 1H), 8.93 (s, 1H), 8.34 (s, 1H), 7.79 (s, 1H), 7.67 (s, 1H), 7.49 (dd, 1H), 7.42 (d, 1H), 7.38 (m, 1H), 6.57 (d, 1H), 4.2 (s, 3H), 3.39 (brs, 2H), 3.23 (m, 2H), 3.08 (dd, 2H), 2.22 (m, 1H), 2.13 (d, (1H); MS m/z 427.1 (M + 1). IC50 (uM): 1.655 |
| 97 | 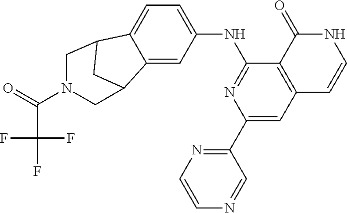 | ¹H NMR (400 MHz, DMSO- d6) δ 12.12 (s, 1H), 8.99 (s, 1H), 8.89 (s, 1H), 7.78 (s, 1H), 7.66 (s, 1H), 7.50 (d, 1H), 7.46 (s, 1H), 7.43 (d, 1H), 4.38 (brm, 1H), 3.94 (m, 1H), 3.59 (m, 1H), 3.43 (m, 2H), 3.18 (brs, 1H), 2.48 (m, 1H), 2.12 (d, 1H); MS m/z 397.4 (M + 1); MS m/z 493.1 (M + 1). IC50 (uM): 2.496 |
| 98 | 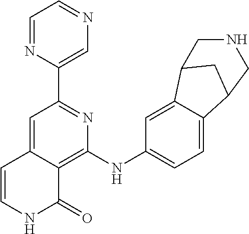 | ¹H NMR (400 MHz, DMSO- d6) δ 12.16 (s, 1H), 8.99 (s, 1H), 8.97 (s, 1H), 7.78 (s, 1H), 7.66 (s, 1H), 7.50 (dd, 1H), 7.46 (s, 1H), 7.43 (d, 1H), 3.42 (brs, 2H), 3.25 (m, 2H), 3.07 (dd, 2H), 2.23 (m, 1H), 2.11 (d, 1H); MS m/z 397.4 (M + 1). IC50 (uM): 1.525 |
| 99 | 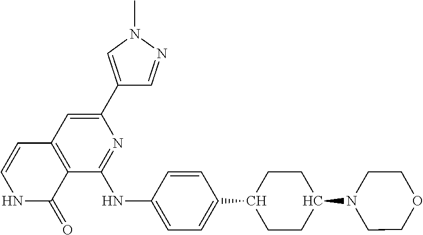 | 1H NMR (400 MHz, DMSO- d6) δ 11.93 (s, 1H), 11.58 (s, 1H), 8.28 (s, 1H), 8.02 (s, 1H), 7.92 (s, 1H), 7.81 (d, 2H), 7.31 (brs, 1H), 7.26 (d, 2H), 6.43 (d, 1H), 3.95 (s, 3H), 3.40 (d, 2H), 3.23- 3.20 (m, 1H), 3.13-3.10 (m, 2H), 2.51- 2.50 (m, 1H), 2.27 (d, 2H), 1.98 (d, 2H), 1.71-1.63 (m, 2H), 1.59-1.55 (m, 2H); MS m/z 485.3 (M + 1). IC50 (uM): 0.168 |
| 100 | 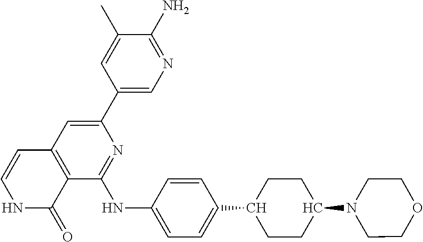 | MS m/z 511.4 (M + 1). IC50 (uM): 0.193 |
| 101 | 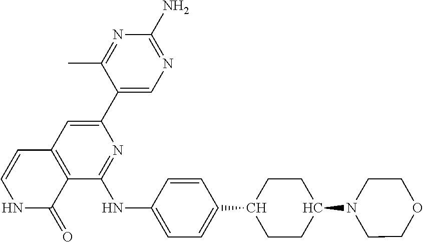 | MS m/z 512.6 (M + 1). IC50 (uM): 0.668 |
| 102 | 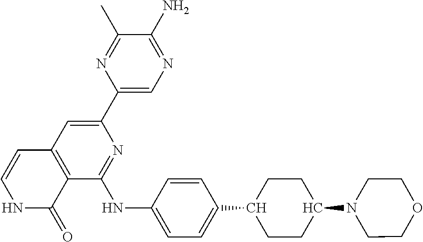 | MS m/z 512.3 (M + 1). IC50 (uM): 0.226 |
| 103 | 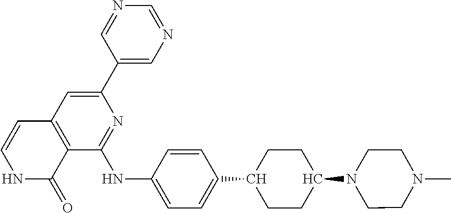 | ¹H NMR (400 MHz, DMSO- d6) δ 11.97 (s, 1H), 11.94 (s, 1H), 9.45 (s, 2H), 9.28 (s, 1H), 7.75 (d, 2H), 7.64 (s, 1H), 7.47 (t, 1H), 7.27 (d, 2H), 6.54 (d, 1H), 3.97 (brs, 4H), 3.40 (d, 2H), 3.23- 3.20 (m, 1H), 3.13-310 (m, 2H), 2.51- 2.50 (m, 4H), 2.27 (d, 2H), 1.98 (d, 2H), 1.71-1.63 (m, 2H), 1.57-1.51 (m, 2H); MS m/z 496.2 (M + 1). IC50 (uM): 0.059 |
| 104 | 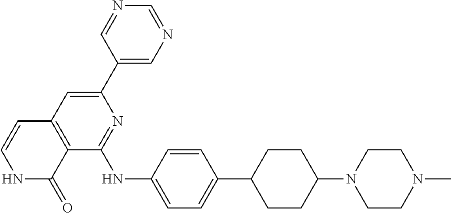 | MS m/z 496.2 (M + 1). IC50 (uM): 0.031 |
| 105 | 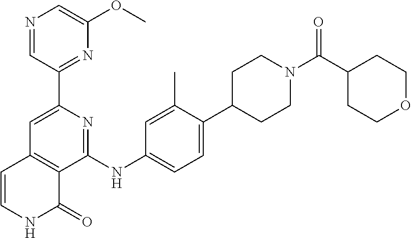 | MS m/z 551.1 (M + 1). IC50 (uM): 0.177 |
| 106 | 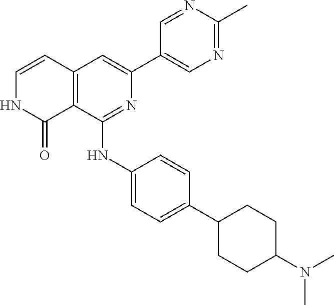 | MS m/z 455.2 (M + 1). IC50 (uM): 0.067 |
| 107 | 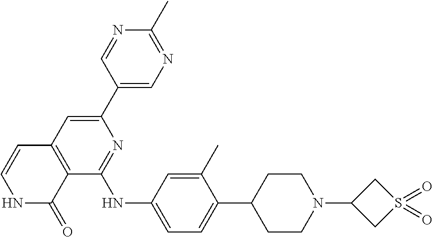 | MS m/z 531.2 (M + 1). IC50 (uM): 0.117 |
| 108 | 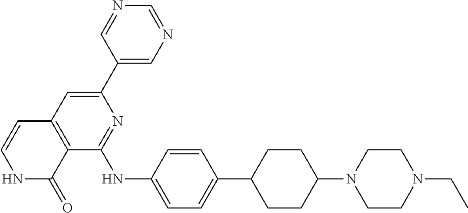 | ¹H NMR (400 MHz, DMSO- d6) δ 12.01 (s, 1H), 11.92 (s, 1H), 9.46 (s, 2H), 9.23 (s, 1H), 7.73 (d, 2H), 7.65 (s, 1H), 7.45 (t, 1H), 7.25 (d, 2H), 6.51 (t, 1H), 3.98 (m, 3H), 3.42 (m, 2H), 3.21- 3.19 (m, 1H), 3.13-3.10 (m, 2H), 2.51-2.50 (m, 4H), 2.27 (d, 2H), 1.98 (d, 2H), 1.71-1.63 (m, 2H), 1.57- 1.51 (m, 2H), 1.04 (d, 3H); MS m/z 510.4 (M + 1). IC50 (uM): 0.116 |
| 109 | 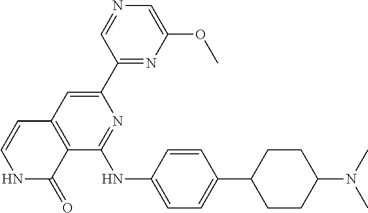 | MS m/z 471.2 (M + 1). IC50 (uM): 0.031 |
| 110 | 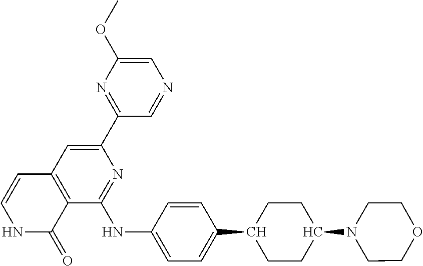 | MS m/z 513.2 (M + 1). |
| 111 | 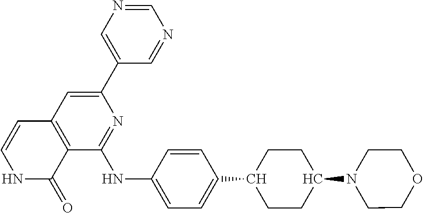 | ¹H NMR (400 MHz, DMSO- d6) δ 11.97 (s, 1H), 11.94 (brs, 1H), 9.45 (s, 2H), 9.28 (s, 1H), 7.76 (d, 2H), 7.64 (s, 1H), 7.47 (t, 1H), 7.27 (d, 2H), 6.55 (d, 1H), 3.96- 3.95 (m, 4H), 3.40 (m, 2H), 3.21-3.10 (m, 2H), 2.27 (d, 2H), 1.98 (d, 2H), 1.98 (d, 2H), 1.71-1.63 (m, 2H), 1.57-1.51 (m, 2H); MS m/z 483.3 (M + 1). IC50 (uM): 0.19 |
| 112 | 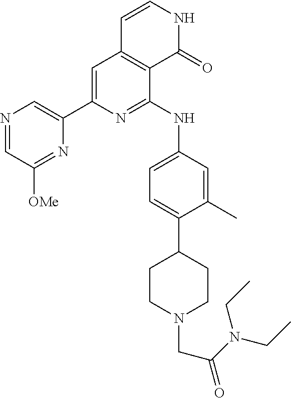 | ESI-MS m/z 570.2 (MH⁺). IC50 (uM): 0.049 |
| 113 | 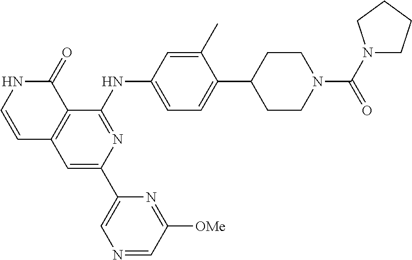 | ESI-MS m/z 540.2 (MH⁺). IC50 (uM): 2.317 |
| 114 | 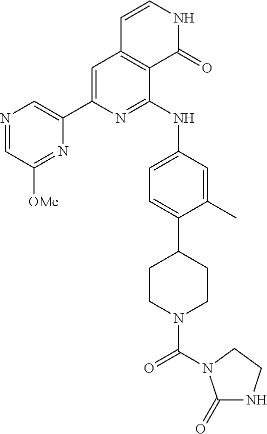 | ¹H NMR (DMSO-d₆) δ 1.17 (bs, 4H), 1.62 (m, 4H), 2.40 (s, 3H), 2.97 (dd, J = 12.8 and 25.2 Hz, 2H), 3.31 (t, J = 7.2 Hz, 2H), 3.66 (t, J = 7.6 Hz, 2H), 4.08 (s, 3H), 4.11 (s, 1H), 6.68 (d, J = 6.8 Hz, 1H), 7.21 (s, 1H), 7.48 (t, J = 6.0 Hz, 1H), 7.67 (d, J = 2.0 Hz, 1H), 7.74 (d, J = 8.4 and 2.0 Hz, 1H), 7.82 (s, 1H), 8.42 (s, 1H), 9.01 (s, 1H), 11.92 (d, J = 5.2 Hz, 1H), 11.96 (s, 1H); ESI-MS m/z 555.2 (MH⁺). IC50 (uM): 0.174 |
| 115 | 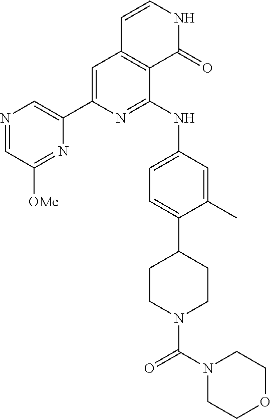 | ¹H NMR (DMSO-d₆) δ 1.17 (bs, 4H), 1.62 (m, 4H), 2.38 (s, 3H), 2.88 (t, J = 12.0 Hz, 3H), 3.16 (t, J = 4.8 Hz, 4H), 3.59 (t, J = 4.4 Hz, 4H), 3.74 (d, J = 13.2 Hz, 2H), 4.07 (s, 3H), 6.67 (dd, J = 1.2 and 7.2 Hz, 1H), 6.24 (d, J = 3.2 Hz, 1H), 7.46 (t, J = 6.8 Hz, 1H), 7.62 (d, J = 2.0 Hz, 1H), 7.76 (dd, J = 8.4 and 2.0 Hz, 1H), 7.80 (s, 1H), 8.41 (s, 1H), 9.07 (s, 1H) 11.89 (d, J = 5.6 Hz, 1H), 11.95 (s, 1H); ESI-MS m/z 556.2 (MH⁺). IC50 (uM): 0.181 |
| 116 | 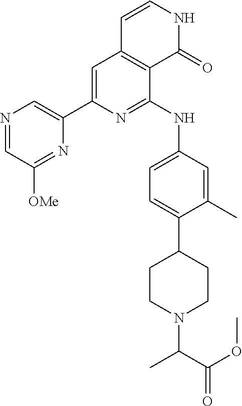 | ¹H NMR (DMSO-d₆) δ 1.56 (d, J = 7.2 Hz, 3H), 2.00 (m, 4H), 2.40 (s, 3H), 3.06-3.60 (m, 5H), 3.84 (s, 3H), 4.09 (s, 3H), 4.40 (d, J = 5.6 Hz, 1H), 6.70 (d, J = 6.8 Hz, 1H), 7.24 (d, J = 8.4 Hz, 1H), 7.49 (t, J = 6.4 Hz, 1H), 7.71 (d, J = 2.0 Hz, 1H), 7.80 (dd, J = 8.4 and 2.0 Hz, 1H), 8.43 (s, 1H), 9.09 (s, 1H), 9.88 (bs, 1H), 11.92 (d, J = 5.6 Hz, 1H), 11.99 (s, 1H); ESI-MS m/z 529.2 (MH⁺). IC50 (uM): 0.184 |
| 117 | 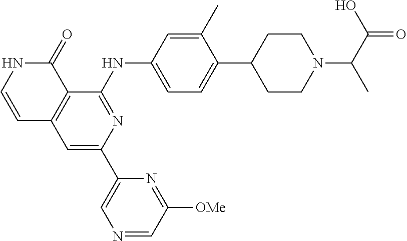 | ESI-MS m/z 515.2 (MH⁺). IC50 (uM): 0.022 |
| 118 | 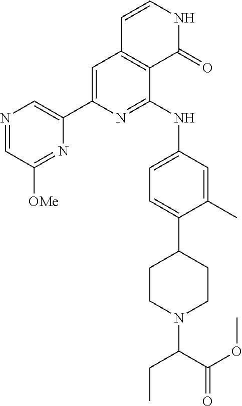 | ¹H NMR (DMSO-d₆) δ 0.95 (t, J = 7.2 Hz, 3H), 2.00 (m, 4H), 2.38 (s, 3H), 3.04-3.70 (m, 5H), 3.86 (s, 3H), 4.05 (s, 3H), 4.15 (dd, J = 8.8 and 4.0 Hz, 1H), 6.61 (d, J = 6.4 Hz, 1H), 7.19 (d, J = 8.4 Hz, 1H), 7.43 (t, J = 6.4 Hz, 1H), 7.70 (dd, J = 19.2 and 2.0 Hz, 1H), 7.75 (s, 1H), 8.37 (s, 1H), 9.02 (s, 1H), 10.13 (bs, 1H), 11.88 (d, J = 6.0 Hz, 1H), 11.95 (s, 1H); ESI-MS m/z 543.2 (MH⁺). EC50 (uM): 0.346 |
| 119 | 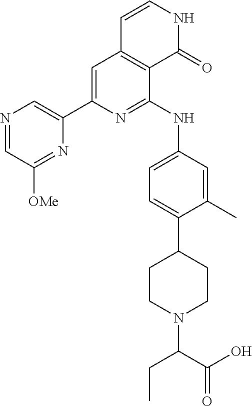 | ¹H NMR (DMSO-d₆) δ 0.99 (t, J = 7.2 Hz, 3H), 2.00 (m, 4H), 2.39 (s, 3H), 3.05-3.70 (m, 5H), 4.05 (s, 3H), 6.68 (dd, J = 7.2 and 1.2 Hz, 1H), 7.23 (bs, 1H), 7.48 (t, J = 6.8 Hz, 1H), 7.70 (s, 1H), 7.80 (d, J = 6.8 Hz, 1H), 7.83 (s, 1H), 8.41 (s, 1H), 9.07 (s, 1H), 9.94 (bs, 1H), 11.92 (d, J = 5.6 Hz, 1H), 11.98 (s, 1H); ESI-MS m/z 529.2 (MH⁺). IC50 (uM): 0.005 |
| 120 | 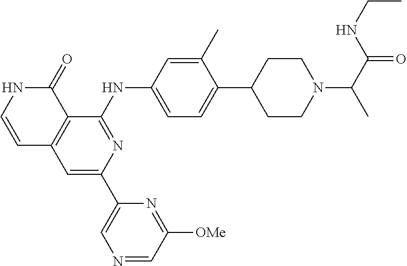 | ESI-MS m/z 542.2 (MH⁺). IC50 (uM): 0.023 |
| 121 | 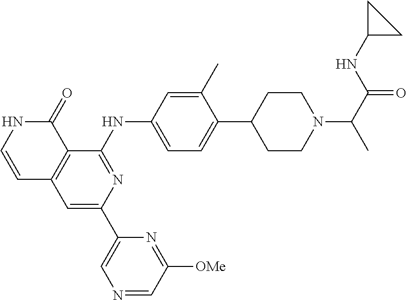 | ESI-MS m/z 554.2 (MH⁺). IC50 (uM): 0.183 |
| 122 | 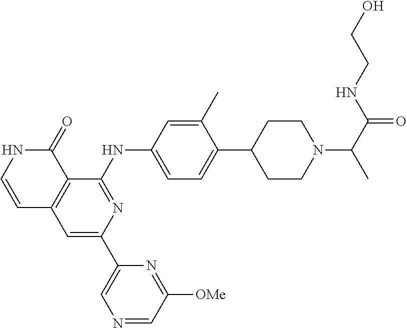 | ESI-MS m/z 558.2 (MH⁺). IC50 (uM): 0.092 |
| 123 | 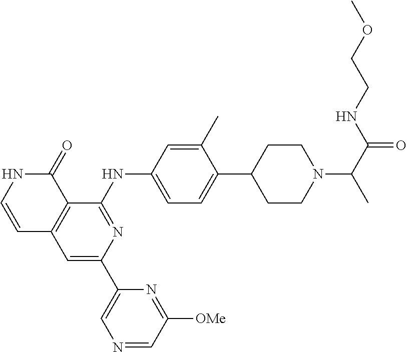 | ESI-MS m/z 572.2 (MH⁺). IC50 (uM): 0.15 |
| 124 | 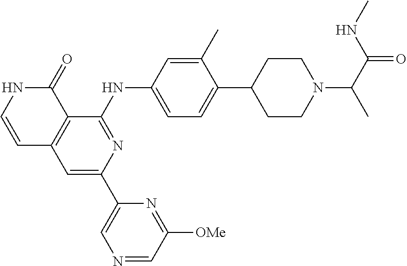 | ESI-MS m/z 542.2 (MH⁺). IC50 (uM): 0.108 |
| 125 | 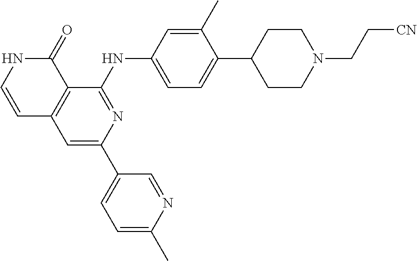 | ESI-MS m/z 579.2 (MH⁺). IC50 (uM): 0.094 |
| 126 | 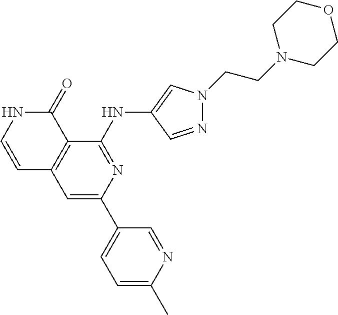 | ESI-MS m/z 432.2 (MH⁺). IC50 (uM): 0.10 |
| 127 | 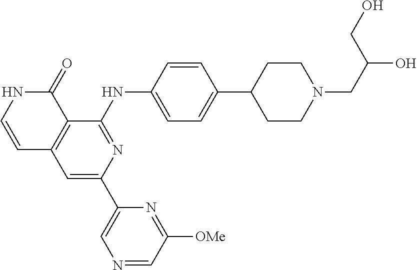 | ESI-MS m/z 503.2 (MH⁺). IC50 (uM): 0.007 |
| 128 | 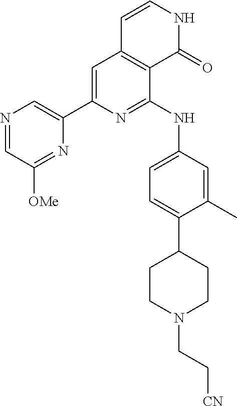 | ¹H NMR (DMSO-d₆) δ 1.98 (s, 3H), 2.40 (s, 3H), 3.03 (bs, 1H), 3.17 (m, 4H), 3.48 (d, J = 4.8 Hz, 2H), 3.60 (d, J = 10.8 Hz, 2H), 4.09 (s, 3H), 6.68 (dd, J = 7.2 and 1.2 Hz, 1H), 7.20 (d, J = 8.4 Hz, 1H), 7.48 (t, J = 6.4 Hz, 1H), 7.70 (s, 1H), 7.78 (d, J = 8.4 Hz, 1H), 7.83 (s, 1H), 8.41 (s, 1H), 9.07 (s, 1H), 10.42 (bs, 1H), 11.92 (d, J = 5.6 Hz, 1H), 11.98 (s, 1H); ESI-MS m/z 496.2 (MH⁺). IC50 (uM): 0.079 |
| 129 | 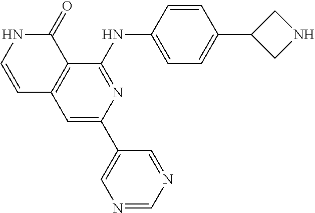 | ESI-MS m/z 371.2 (MH⁺). IC50 (uM): 0.109 |
| 130 | 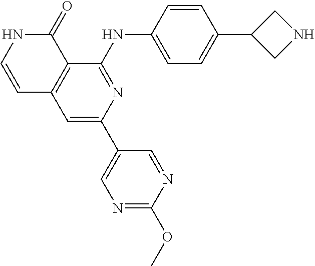 | ESI-MS m/z 401.2 (MH⁺). IC50 (uM): 0.057 |
| 131 | 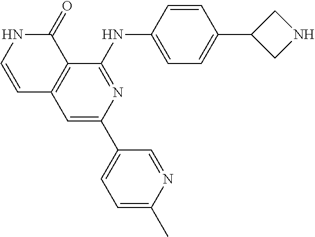 | ESI-MS m/z 384.2 (MH⁺). IC50 (uM): 0.111 |
| 132 | 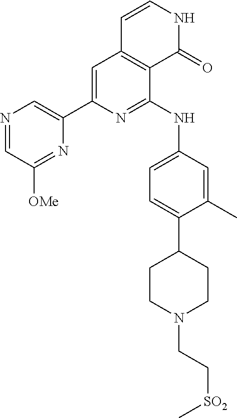 | ¹H NMR (DMSO-d₆) δ 1.68 (m, 4H), 2.10 (dd, J = 11.2 and 8.8 Hz, 1H), (s, 3H), 2.36 (s, 3H), 2.67 (bs, 1H), 2.75 (t, J = 6.8 Hz, 2H), 3.03 (d, J = 11.2 Hz, 2H), 3.08 (s, 3H), 3.31 (d, J = 6.8 Hz, 2H), 3.34 (s, 3H), 4.08 (s, 3H), 6.67 (d, J = 7.2 Hz, 1H), 7.26 (d, J = 8.4 Hz, 1H), 7.46 (d, J = 7.2 Hz, 1H), 7.63 (d, J = 2.0 Hz, 1H), 7.75 (dd, J = 8.4 and 2.4 Hz, 1H), 7.81 (s, 1H), 8.41 (s, 1H), 9.07 (s, 1H), 11.87 (bs, 1H), 11.94 (s, 1H); ESI-MS m/z 549.2 (MH⁺). IC50 (uM): 0.115 |
| 133 | 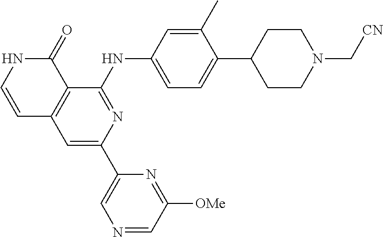 | ESI-MS m/z 482.2 (MH⁺). IC50 (uM): 0.13 |
| 134 | 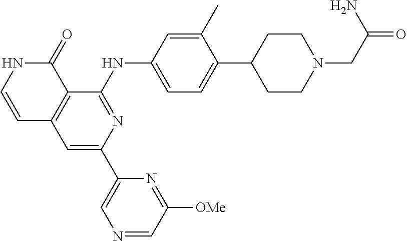 | ESI-MS m/z 500.2 (MH⁺). IC50 (uM): 0.04 |
| 135 | 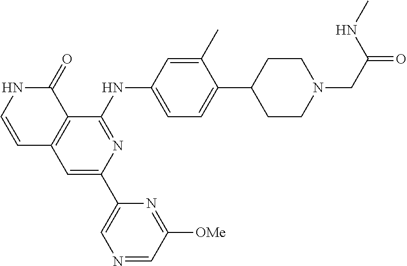 | ESI-MS m/z 514.2 (MH⁺). IC50 (uM): 0.048 |
| 136 | 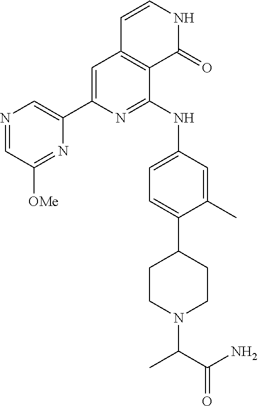 | ¹H NMR (DMSO-d₆) δ 1.11 (d, J = 6.8 Hz, 3H), 1.70 (bs, 4H), 2.23 (m, 1H), 2.38 (s, 3H), 2.40 (m, 1H), 2.67 (m, 1H), 2.86 (d, J = 10.8 Hz, 1H), 2.90 (d, J = 11.2 Hz, 1H), 3.05 (dd, J = 13.6 and 6.8 Hz, 1H), 4.09 (s, 3H), 6.67 (d, J = 6.8 Hz, 1H), 7.03 (s, 1H), 7.29 (s, 1H), 7.31 (s, 1H), 7.46 (d, J = 6.0 Hz, 1H), 7.61 (d, J = 2.0 Hz, 1H), 7.77 (dd, J = 8.4 and 2.4 Hz, 1H), 7.81 (s, 1H), 8.42 (s, 1H), 9.08 (s, 1H), 11.89 (bs, 1H), 11.94 (s, 1H); ESI-MS m/z 514.2 (MH⁺). IC50 (uM): 0.046 |
| 137 | 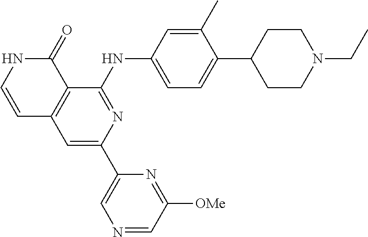 | ESI-MS m/z 471.2 (MH⁺). IC50 (uM): 0.01 |
| 138 | 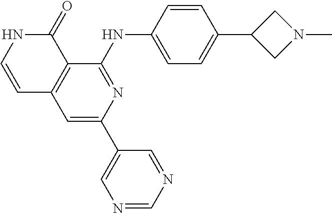 | ESI-MS m/z 385.2 (MH⁺). IC50 (uM): 0.026 |
| 139 | 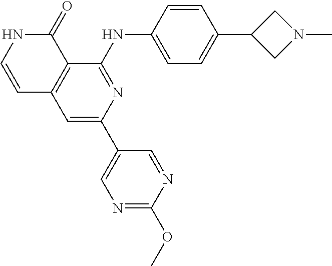 | ESI-MS m/z 415.2 (MH⁺). IC50 (uM): 0.034 |
| 140 | 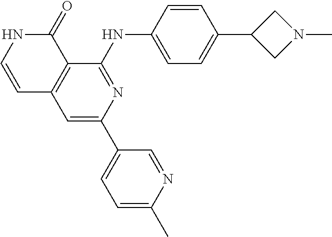 | ESI-MS m/z 398.2 (MH⁺). IC50 (uM): 0.027 |
| 141 | 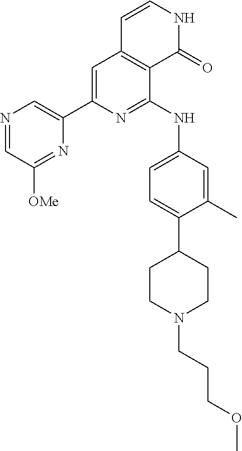 | ¹H NMR (DMSO-d₆) δ 1.99 (m, 6H), 2.40 (s, 3H), 3.11 (m, 5H), 3.27 (s, 3H), 3.42 (t, J = 6.0 Hz, 2H), 3.58 (d, J = 11.2 Hz, 2H), 4.08 (s, 3H), 6.68 (d, J = 6.8 Hz, 1H), 7.21 (d, J = 8.0 Hz, 1H), 7.48 (t, J = 6.4 Hz, 1H), 7.71 (d, J = 2.0 Hz, 1H), 7.76 (dd, J = 8.0 and 2.0 Hz, 1H), 7.83 (s, 1H), 8.41 (s, 1H), 9.08 (s, 1H), 10.17 (bs, 1H), 11.93 (d, J = 5.6 Hz, 1H), 11.98 (s, 1H); ESI-MS m/z 515.2 (MH⁺). IC50 (uM): 0.007 |
| 142 | 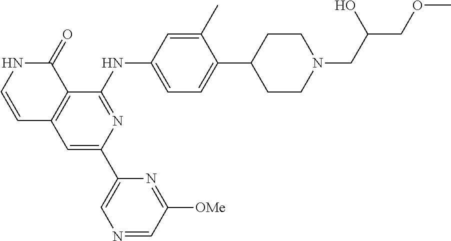 | ESI-MS m/z 531.2 (MH⁺). IC50 (uM): 0.007 |
| 143 | 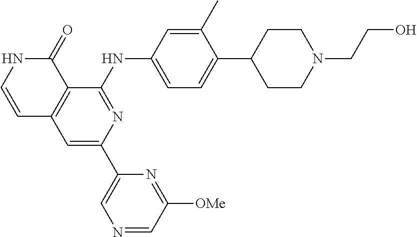 | ESI-MS m/z 487.2 (MH⁺). IC50 (uM): 0.005 |
| 144 | 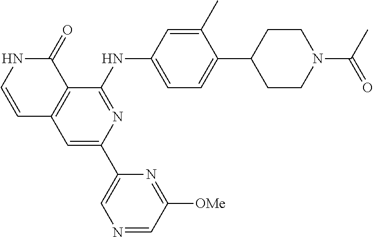 | ESI-MS m/z 485.2 (MH⁺). IC50 (uM): 0.092 |
| 145 | 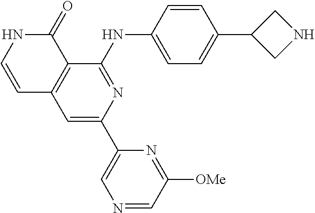 | ESI-MS m/z 401.2 (MH⁺). IC50 (uM): 0.006 |
| 146 | 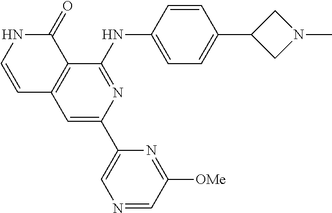 | ESI-MS m/z 415.2 (MH⁺). IC50 (uM): 0.004 |
| 147 | 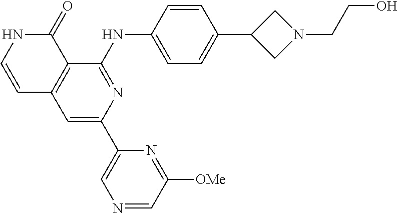 | ESI-MS m/z 445.2 (MH⁺). IC50 (uM): 0.004 |
| 148 | 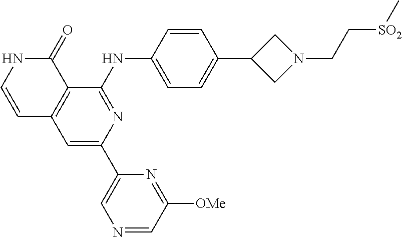 | ESI-MS m/z 507.2 (MH⁺). IC50 (uM): 0.022 |
| 149 | 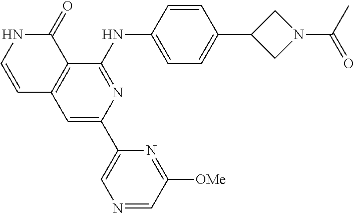 | ESI-MS m/z 443.2 (MH⁺). IC50 (uM): 0.024 |
| 150 | 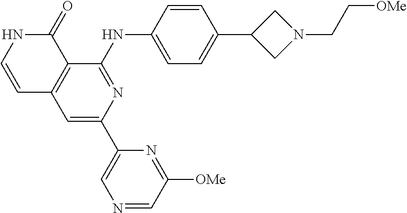 | ESI-MS m/z 459.2 (MH⁺). IC50 (uM): 0.083 |
| 151 | 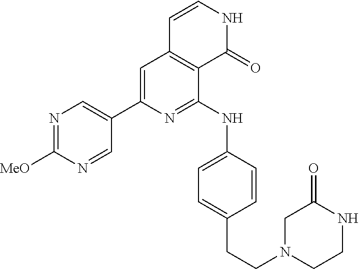 | ESI-MS m/z 472.2 (MH⁺). IC50 (uM): 0.201 |
| 152 | 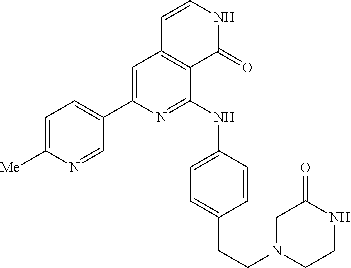 | ESI-MS m/z 455.2 (MH⁺). IC50 (uM): 0.117 |
| 153 | 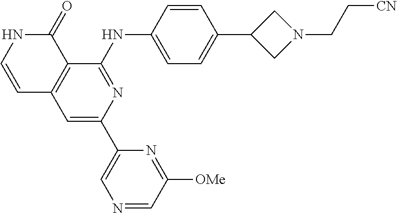 | ¹H NMR (DMSO-d₆) δ 2.55 (t, J = 6.0 Hz, 2H), 2.68 (t, J = 6.8 Hz, 2H), 3.15 (t, J = 6.4 Hz, 2H), 3.67 (m, 3H), 4.08 (s, 3H), 6.68 (d, J = 6.8 Hz, 1H), 7.40 (d, J = 8.4 Hz, 2H), 7.48 (d, J = 6.8 Hz, 1H), 7.83 (s, 2H), 7.86 (s, 1H), 8.42 (s, 1H), 9.07 (s, 1H), 11.87 (bs, 1H), 12.01 (s, 1H); ESI-MS m/z 454.2 (MH⁺). IC50 (uM): 0.032 |
| 154 | 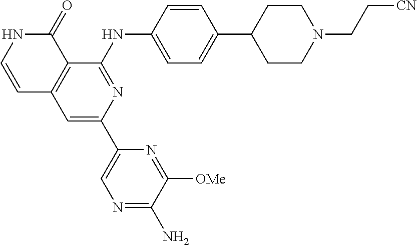 | ESI-MS m/z 497.2 (MH⁺). IC50 (uM): 0.02 |
| 155 | 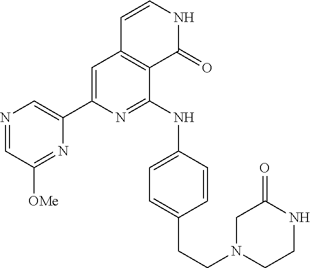 | ESI-MS m/z 472.2 (MH⁺). IC50 (uM): 0.055 |
| 156 | 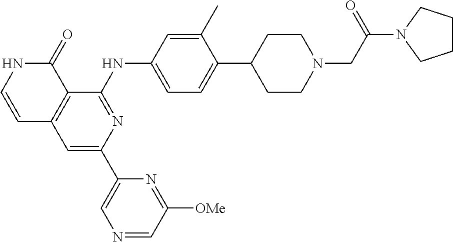 | ESI-MS m/z 554.2 (MH⁺). IC50 (uM): 0.041 |
| 157 | 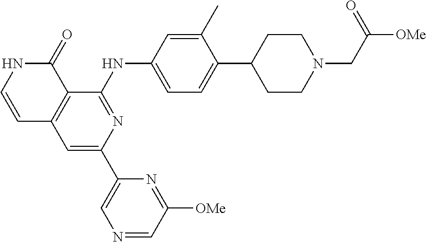 | ESI-MS m/z 515.2 (MH⁺). IC50 (uM): 0.099 |
| 158 | 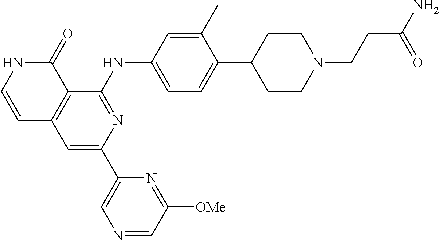 | ESI-MS m/z 514.2 (MH⁺). IC50 (uM): 0.024 |
| 159 | 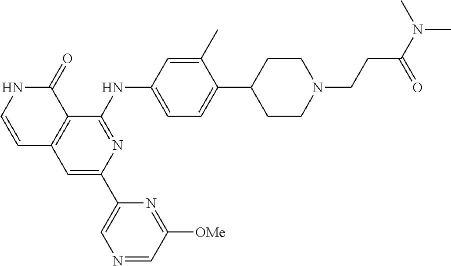 | ESI-MS m/z 542.2 (MH⁺). IC50 (uM): 0.038 |
| 160 | 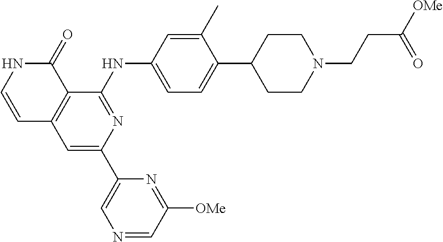 | ESI-MS m/z 529.2 (MH⁺). IC50 (uM): 0.081 |
| 161 | 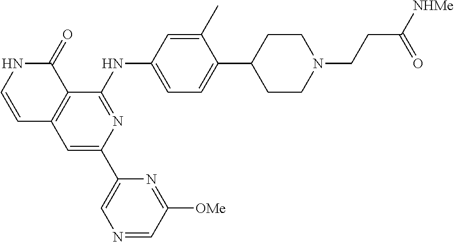 | ESI-MS m/z 528.2 (MH⁺). IC50 (uM): 0.018 |
| 162 | 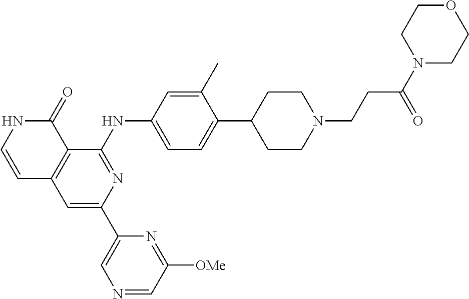 | ESI-MS m/z 584.2 (MH⁺). IC50 (uM): 0.032 |
| 163 | 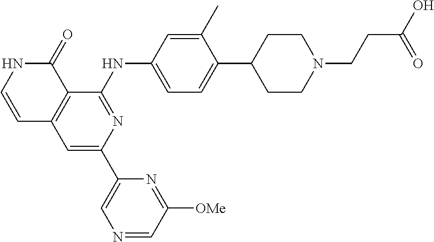 | ESI-MS m/z 515.2 (MH⁺). IC50 (uM): 0.007 |
| 164 | 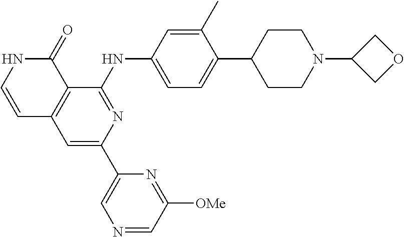 | ESI-MS m/z 499.2 (MH⁺). IC50 (uM): 0.287 |
| 165 | 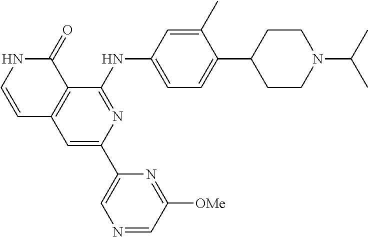 | ESI-MS m/z 485.2 (MH⁺). IC50 (uM): 0.003 |
| 166 | 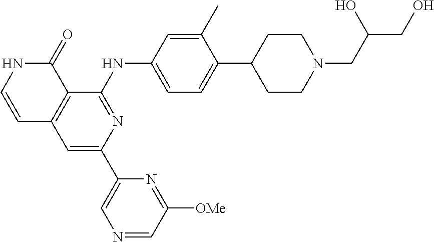 | ESI-MS m/z 517.2 (MH⁺). IC50 (uM): 0.003 |
| 167 | 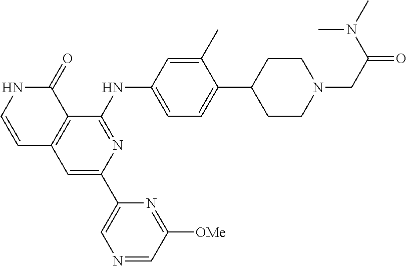 | ESI-MS m/z 528.2 (MH⁺). IC50 (uM): 0.091 |
| 168 | 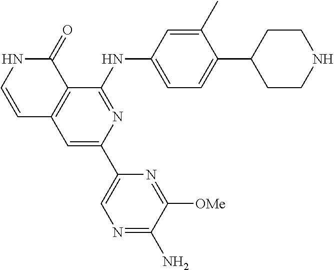 | ESI-MS m/z 458.2 (MH⁺). IC50 (uM): 0.004 |
| 169 | 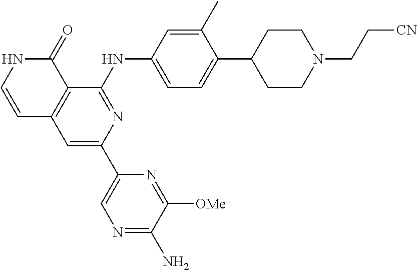 | ESI-MS m/z 511.2 (MH⁺). IC50 (uM): 0.029 |
| 170 | 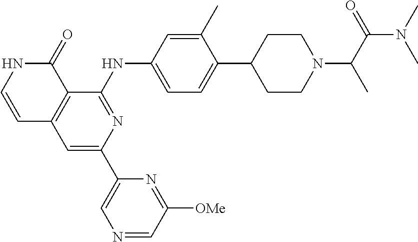 | ESI-MS m/z 542.2 (MH⁺). IC50 (uM): 0.006 |
| 171 | 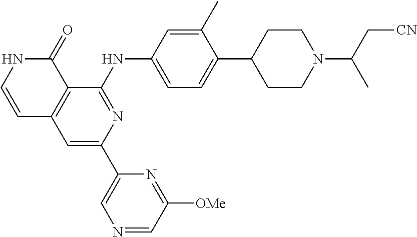 | ESI-MS m/z 510.2 (MH⁺). IC50 (uM): 0.16 |
| 172 | 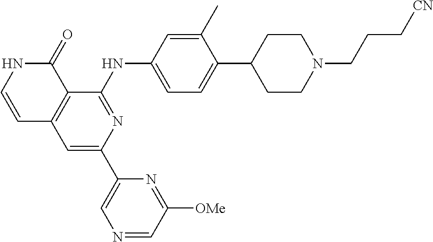 | ESI-MS m/z 510.2 (MH⁺). IC50 (uM): 0.019 |
| 173 | 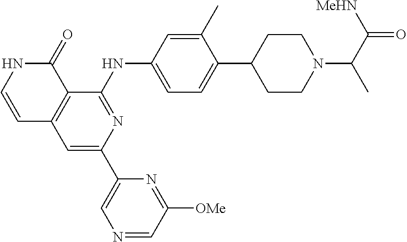 | ESI-MS m/z 528.2 (MH⁺). IC50 (uM): 0.011 |
| 174 | 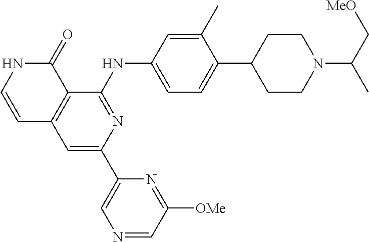 | ESI-MS m/z 515.2 (MH⁺). IC50 (uM): 0.014 |
| 175 | 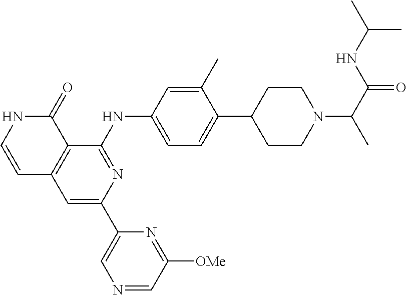 | ESI-MS m/z 556.2 (MH⁺). IC50 (uM): 0.144 |
| 176 | 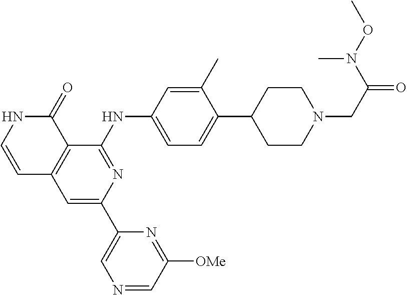 | ESI-MS m/z 544.2 (MH⁺). IC50 (uM): 0.041 |
| 177 | 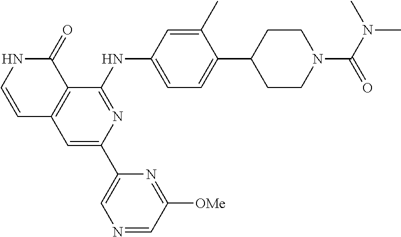 | ESI-MS m/z 514.2 (MH⁺). IC50 (uM): 0.496 |
| 178 | 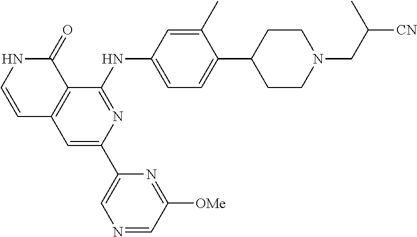 | ESI-MS m/z 510.2 (MH⁺). IC50 (uM): 0.061 |
| 179 | 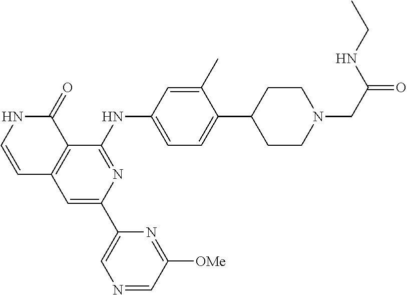 | ESI-MS m/z 528.2 (MH⁺). IC50 (uM): 0.08 |
| 180 | 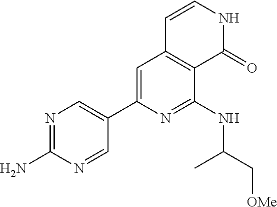 | ESI-MS m/z 327.2 (MH⁺). IC50 (uM): 0.115 |
| 181 | 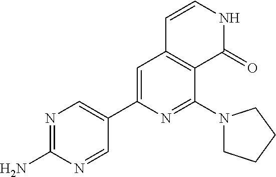 | ESI-MS m/z 309.2 (MH⁺). IC50 (uM): 0.015 |
| 182 | 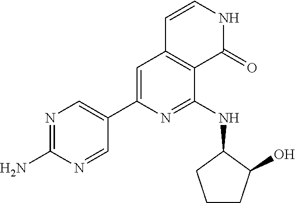 | ESI-MS m/z 339.2 (MH⁺). IC50 (uM): 0.03 |
| 183 | 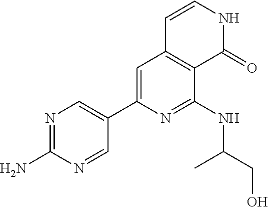 | ¹H NMR (DMSO-d₆) δ 1.16 (d, J = 6.0 Hz, 3H), 3.47 (dd, J = 13.2 and 6.8 Hz, 1H), 3.68 (dd, J = 13.6 and 3.6 Hz, 1H), 3.93 (m, 1H), 6.43 (d, J = 6.8 Hz, 1H), 7.19 (s, 1H), 7.46 (s, 1H), 7.89 (bs, 1H), 8.97 (s, 2H), 11.81 (bs, 1H); ESI-MS m/z 313.2 (MH⁺). IC50 (uM): 0.203 |
| 184 | 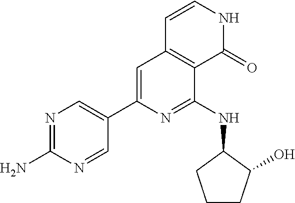 | ESI-MS m/z 339.2 (MH⁺). IC50 (uM): 0.059 |
| 185 | 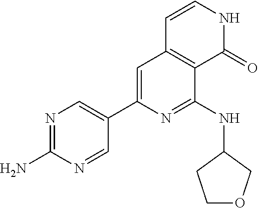 | ESI-MS m/z 325.2 (MH⁺). IC50 (uM): 0.145 |
| 186 | 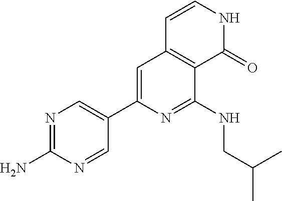 | ¹H NMR (DMSO-d₆) δ 0.98 (d, J = 6.4 Hz, 6H), 1.97 (m, 1H), 3.39 (t, J = 6.0 Hz, 2H), 3.35 (d, J = 6.0 Hz, 1H), 7.09 (s, 1H), 7.17 (bs, 1H), 7.30 (t, J = 6.4 Hz, 1H), 8.95 (s, 2H), 9.55 (bs, 1H), 11.41 (bs, 1H); ESI-MS m/z 311.2 (MH⁺). IC50 (uM): 0.178 |
| 187 | 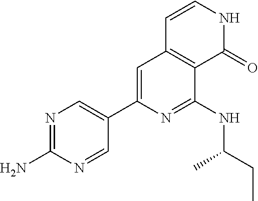 | ESI-MS m/z 311.2 (MH⁺). IC50 (uM): 0.301 |
| 188 | 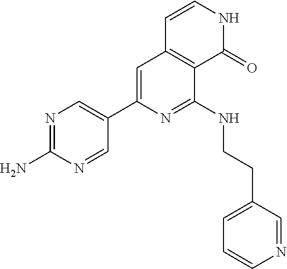 | ESI-MS m/z 360.2 (MH⁺). IC50 (uM): 0.093 |
| 189 | 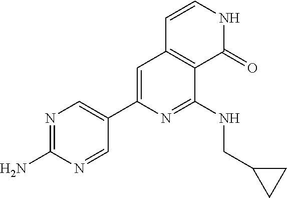 | ESI-MS m/z 309.2 (MH⁺). IC50 (uM): 0.384 |
| 190 | 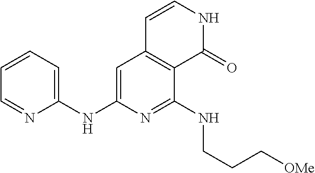 | ¹H NMR (MeOD-d₄) δ 2.08 (m, 2H), 3.35 (s, 3H), 3.60 (t, J = 5.6 Hz, 2H), 3.75 (t, J = 6.8 Hz, 2H), 6.19 (s, 1H), 6.40 (d, J = 7.2 Hz, 1H), 7.25 (m, 2H), 7.33 (d, J = 6.8 Hz, 1H), 8.08 (m, 1H), 8.37 (m, 1H); ESI-MS m/z 326.2 (MH⁺). IC50 (uM): 0.286 |
| 191 | 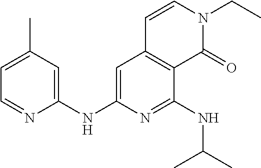 | ¹H NMR (DMSO-d₆) δ 1.24 (t, J = 7.2 Hz, 3H), 1.36 (d, J = 6.4 Hz, 6H), 2.44 (s, 1H), 3.92 (q, J = 7.2 Hz, 2H), 4.15 (m, 1H), 6.31 (s, 1H), 6.49 (d, J = 7.2 Hz, 1H), 7.12 (d, J =6.0 Hz, 1H), 7.35 (s, 1H), 7.66 (d, J = 7.6 Hz, 1H), 8.31 (d, J = 6.0 Hz, 1H), 10.04 (d, J = 6.4 Hz, 1H), 11.62 (bs, 1H); ESI-MSm/z 338.2 (MH⁺). IC50 (uM): 0.187 |
| 192 | 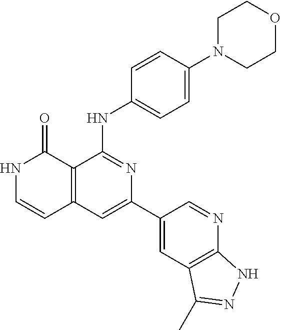 | ESI-MS m/z 454.2 (MH⁺). IC50 (uM): 0.263 |
| 193 | 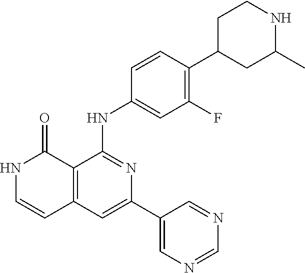 | ESI-MS m/z 430.2 (MH⁺). IC50 (uM): 0.043 |
| 194 | 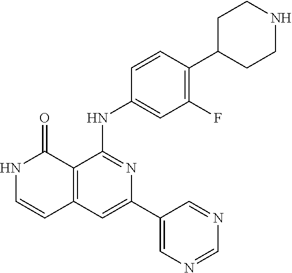 | ESI-MS m/z 417.2 (MH⁺). IC50 (uM): 0.028 |
| 195 | 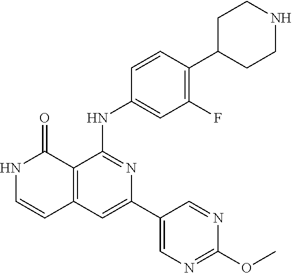 | ESI-MS m/z 445.2 (MH⁺). IC50 (uM): 0.049 |
| 196 | 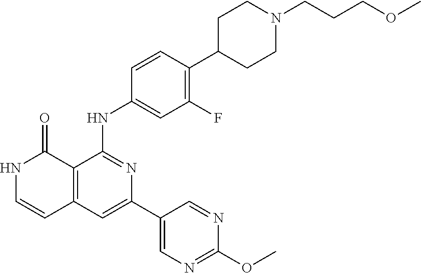 | ESI-MS m/z 519.2 (MH⁺). IC50 (uM): 0.059 |
| 197 | 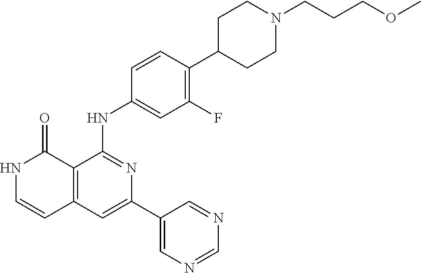 | ESI-MS m/z 489.2 (MH⁺). IC50 (uM): 0.035 |
| 198 | 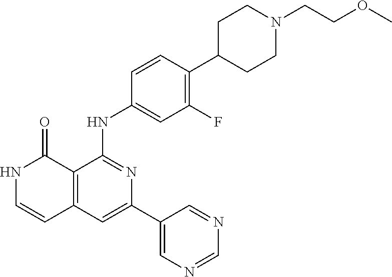 | ESI-MS m/z 475.2 (MH⁺). IC50 (uM): 0.031 |
| 199 | 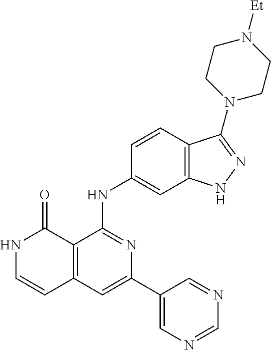 | MS m/z 468.1 (M + 1). IC50 (uM): 0.020 |
| 200 | 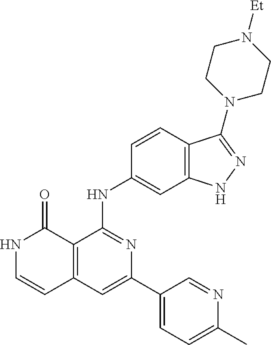 | MS m/z 481.2 (M + 1). IC50 (uM): 0.056 |
| 201 | 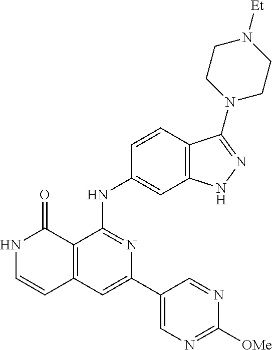 | MS m/z 498.2 (M + 1). IC50 (uM): 0.070 |
| 202 | 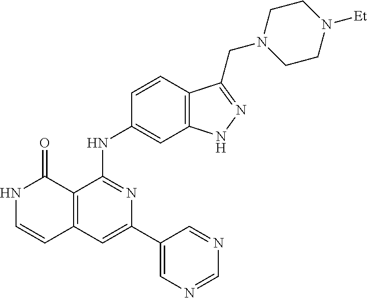 | MS m/z 482.2 (M + 1). IC50 (uM): 0.027 |
| 203 | 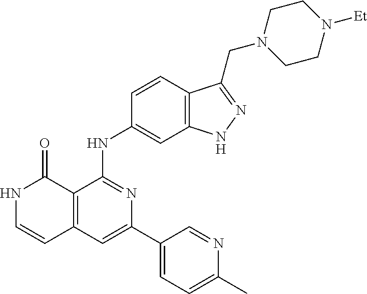 | MS m/z 495.2 (M + 1). IC50 (uM): 0.049 |
| 204 | 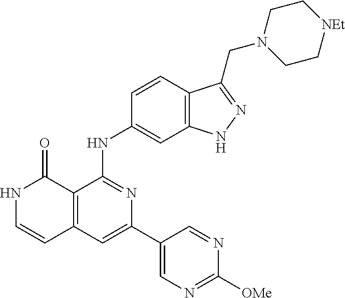 | MS m/z 512.2 (M + 1). IC50 (uM): 0.048 |
| 205 | 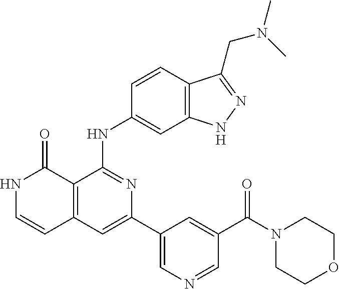 | MS m/z 525.2 (M + 1). IC50 (uM): 0.104 |
| 206 | 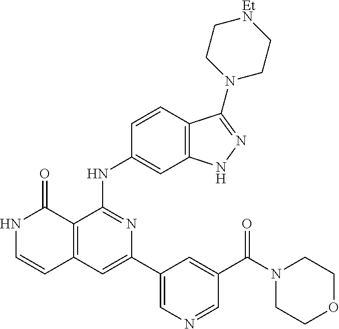 | MS m/z 580.3 (M + 1). IC50 (uM): 0.06 |
| 207 | 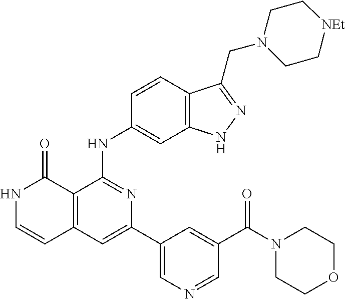 | MS m/z 594.3 (M + 1). IC50 (uM): 0.089 |
| 208 | 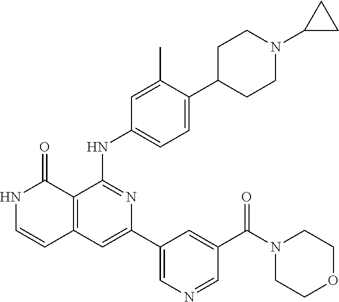 | MS m/z 565.3 (M + 1). IC50 (uM): 0.112 |
| 209 | 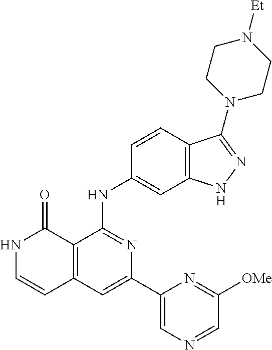 | MS m/z 498.2 (M + 1). IC50 (uM): 0.011 |
| 210 | 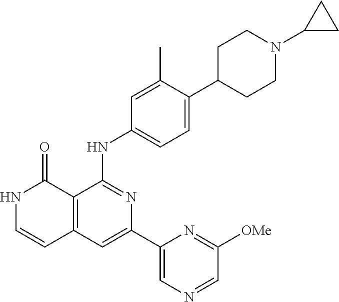 | MS m/z 483.2 (M + 1). IC50 (uM): 0.088 |
| 211 | 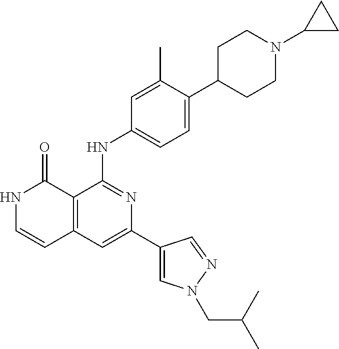 | MS m/z 497.3 (M + 1). IC50 (uM): 0.197 |
| 212 | 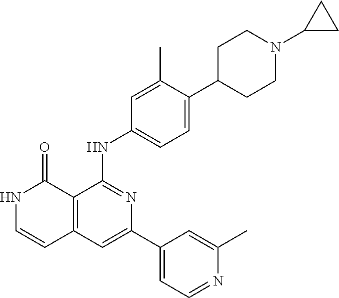 | MS m/z 466.2 (M + 1). IC50 (uM): 0.39 |
| 213 | 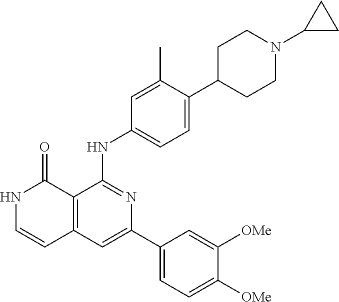 | MS m/z 511.3 (M + 1). IC50 (uM): 0.99 |
| 214 | 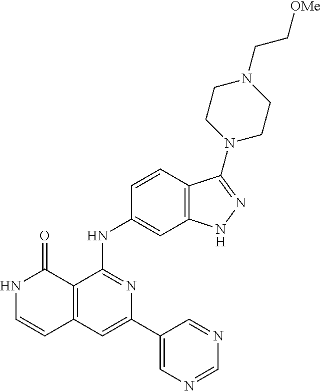 | MS m/z 498.2 (M + 1). IC50 (uM): 0.017 |
| 215 | 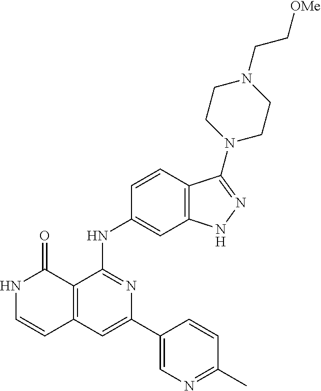 | MS m/z 511.2 (M + 1). IC50 (uM): 0.067 |
| 216 | 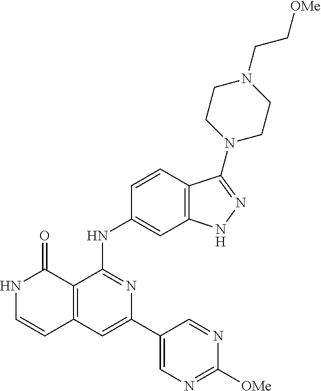 | MS m/z 528.2 (M + 1). IC50 (uM): 0.037 |
| 217 | 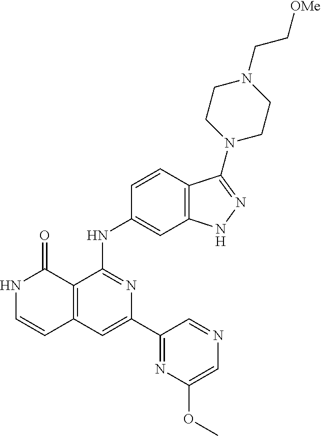 | MS m/z 528.3 (M + 1). IC50 (uM): 0.029 |
| 218 | 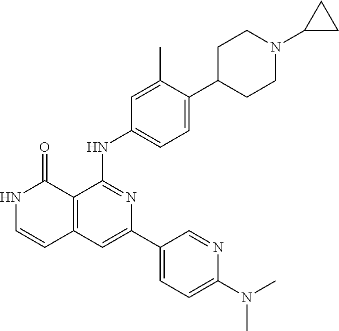 | MS m/z 495.3 (M + 1). IC50 (uM): 0.35 |
| 219 | 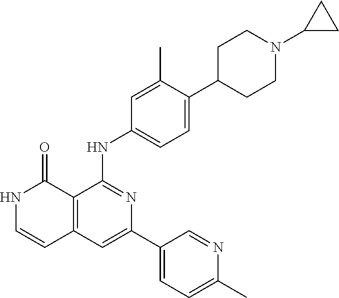 | MS m/z 466.3 (M + 1). IC50 (uM): 0.089 |
| 220 | 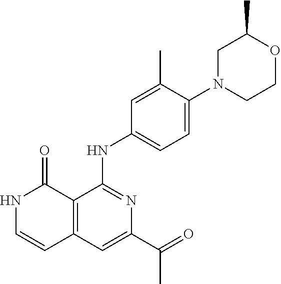 | MS m/z 393.2 (M + 1). IC50 (uM): 0.807 |
| 221 | 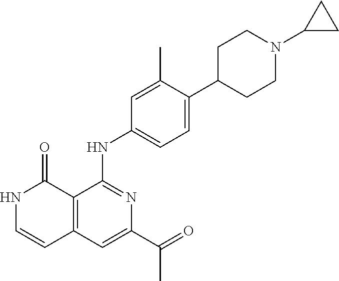 | MS m/z 417.2 (M + 1). IC50 (uM): 0.253 |
| 222 | 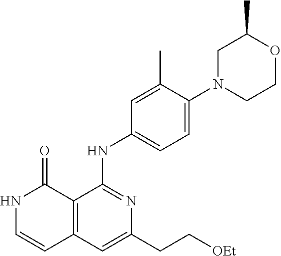 | MS m/z 423.2 (M + 1). IC50 (uM): 0.243 |
| 223 | 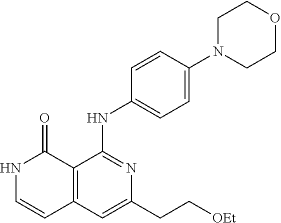 | MS m/z 396.2 (M + 1). IC50 (uM): 0.149 |
| 224 | 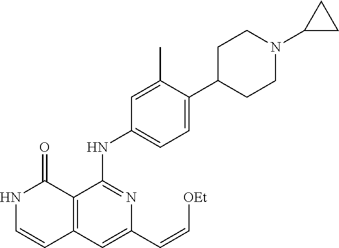 | MS m/z 423.2 (M + 1). IC50 (uM): 0.297 |
| 225 | 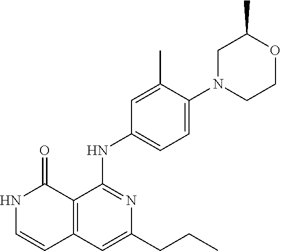 | MS m/z 393.2 (M + 1). IC50 (uM): 2.30 |
| 226 | 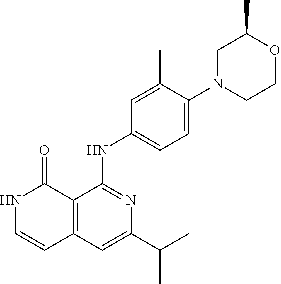 | MS m/z 393.2 (M + 1). IC50 (uM): 1.744 |
| 227 | 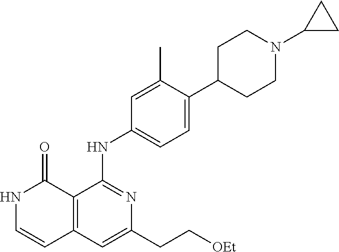 | MS m/z 447.3 (M + 1). IC50 (uM): 0.398 |
| 228 | 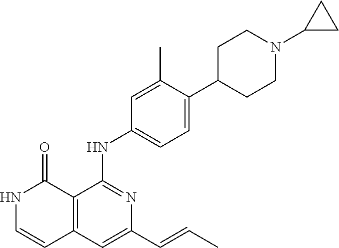 | MS m/z 415.2 (M + 1). IC50 (uM): 1.66 |
| 229 | 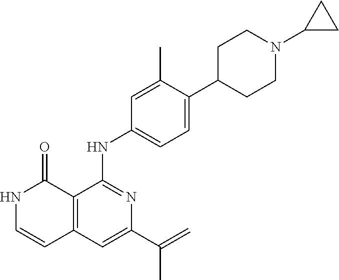 | MS m/z 415.2 (M + 1). IC50 (uM): 0.743 |
| 230 | 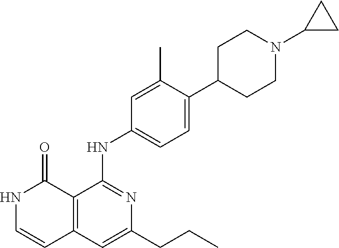 | MS m/z 417.3 (M + 1). IC50 (uM): 0.556 |
| 231 | 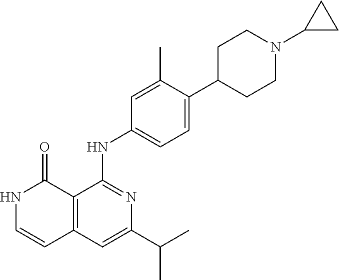 | MS m/z 417.3 (M + 1). IC50 (uM): 0.864 |
| 232 | 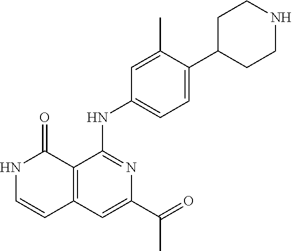 | MS m/z 377.2 (M + 1). IC50 (uM): 0.242 |
| 233 | 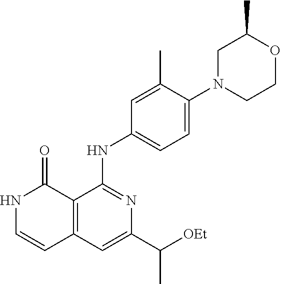 | MS m/z 423.2 (M + 1). IC50 (uM): 0.754 |
| 234 | 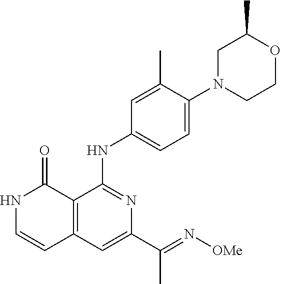 | MS m/z 422.2 (M + 1). IC50 (uM): 1.306 |
| 235 | 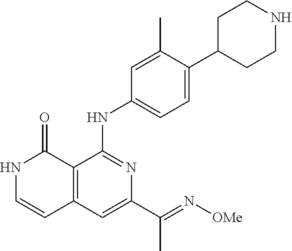 | MS m/z 406.2 (M + 1). IC50 (uM): 0.102 |
| 236 | 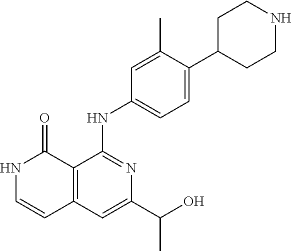 | MS m/z 379.2 (M + 1). IC50 (uM): 0.012 |
| 237 | 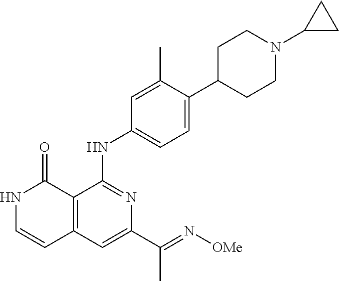 | MS m/z 446.2 (M + 1). IC50 (uM): 0.407 |
| 238 | 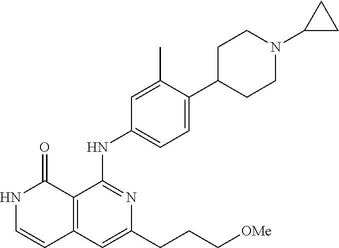 | MS m/z 447.3 (M + 1). IC50 (uM): 1.288 |
| 239 | 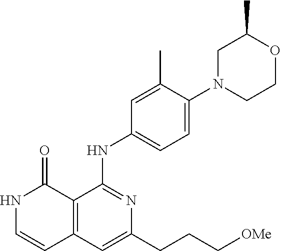 | MS m/z 423.2 (M + 1). IC50 (uM): 2.224 |
| 240 | 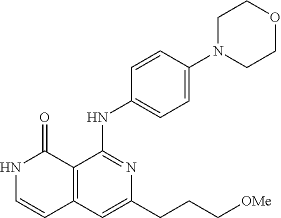 | MS m/z 395.2 (M + 1). IC50 (uM): 0.356 |
| 241 | 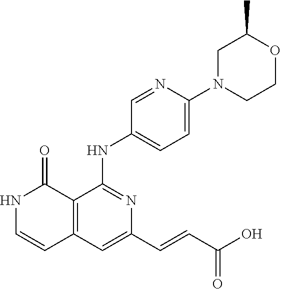 | MS m/z 408.2 (M + 1). IC50 (uM): 0.208 |
| 242 | 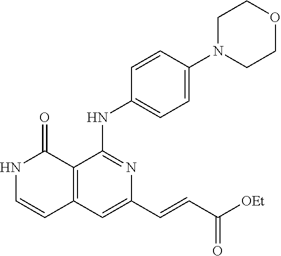 | MS m/z 421.2 (M + 1). IC50 (uM): 0.332 |
| 243 | 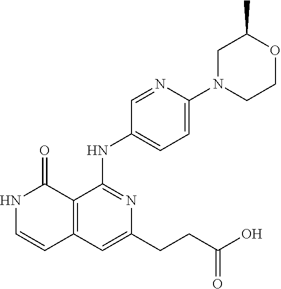 | MS m/z 410.2 (M + 1). IC50 (uM): 0.307 |
| 244 | 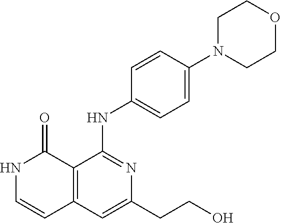 | MS m/z 367.2 (M + 1). IC50 (uM): 0.031 |
| 245 | 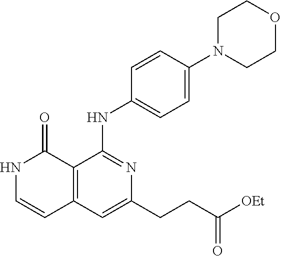 | MS m/z 423.2 (M + 1). IC50 (uM): 0.295 |
| 246 | 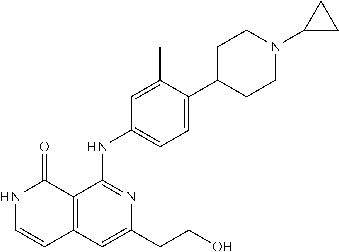 | MS m/z 419.2 (M + 1). IC50 (uM): 0.041 |
| 247 | 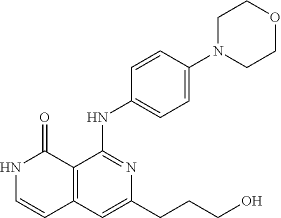 | MS m/z 381.2 (M + 1). IC50 (uM): 0.045 |
| 248 | 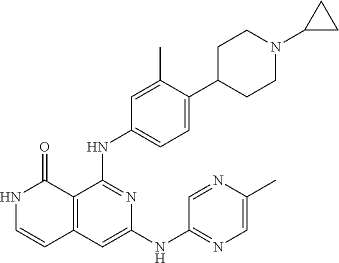 | MS m/z 482.3 (M + 1). IC50 (uM): 2.02 |
| 249 | 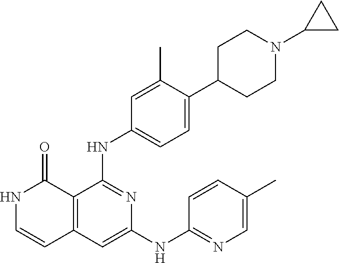 | MS m/z 481.3 (M + 1). IC50 (uM): 1.589 |
| 250 | 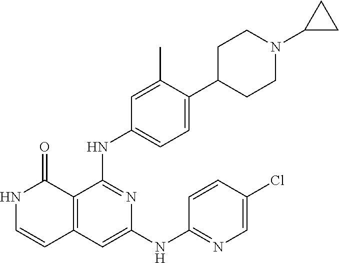 | MS m/z 501.2 (M + 1). IC50 (uM): 2.083 |
| 251 | 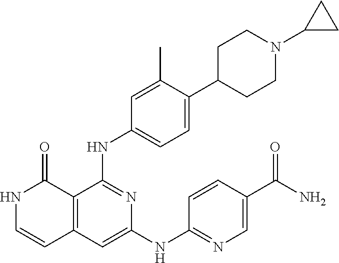 | MS m/z 510.2 (M + 1). IC50 (uM): 0.058 |
| 252 | 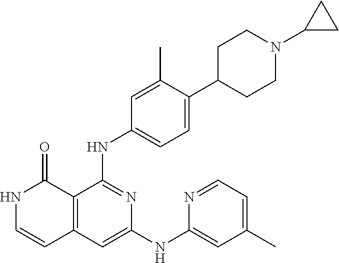 | MS m/z 481.3 (M + 1). IC50 (uM): 0.032 |
| 253 | 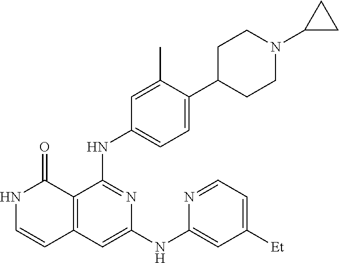 | MS m/z 495.3 (M + 1). IC50 (uM): 0.033 |
| 254 | 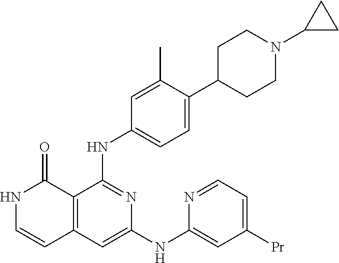 | MS m/z 509.3 (M + 1) |
| 255 | 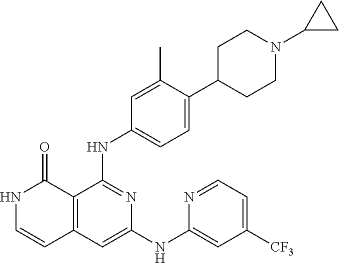 | MS m/z 535.2 (M + 1). IC50 (uM): 0.434 |
| 256 | 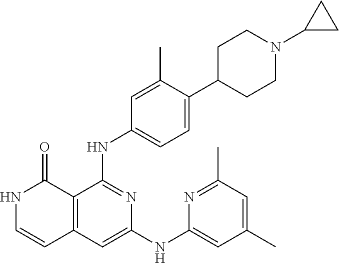 | MS m/z 495.3 (M + 1). IC50 (uM): 0.141 |
| 257 | 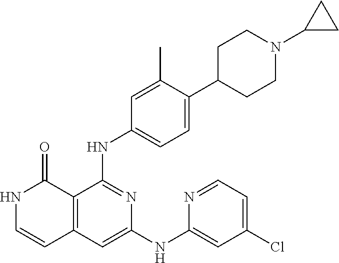 | MS m/z 501.2 (M + 1). IC50 (uM): 0.17 |
| 258 | 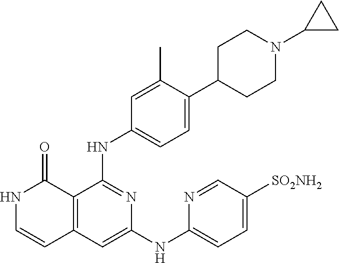 | MS m/z 546.2 (M + 1). IC50 (uM): 0.061 |
| 259 | 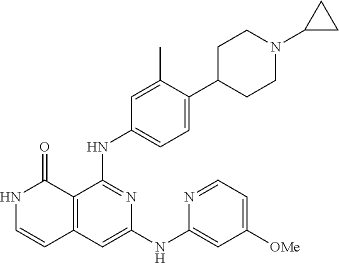 | MS m/z 497.3 (M + 1). IC50 (uM): 0.255 |
| 260 | 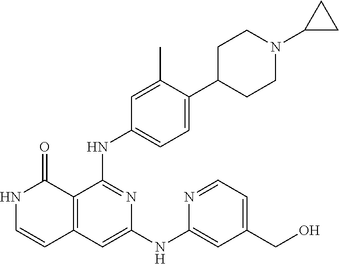 | MS m/z 497.3 (M + 1). IC50 (uM): 0.049 |
| 261 | 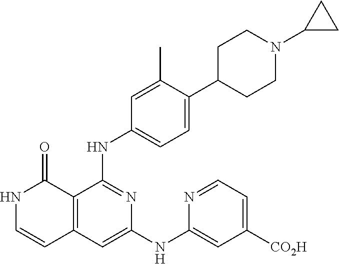 | MS m/z 511.2 (M + 1). IC50 (uM): 0.249 |
| 262 | 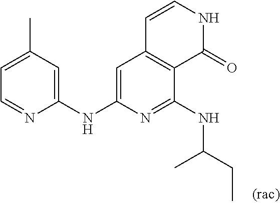 | ¹H NMR (DMSO-d₆) δ 0.98 (t, J = 7.2 Hz, 3H), 1.31 (d, J = 6.4 Hz, 3H), 1.68 (m, 2H), 2.43 (s, 3H), 4.01 (m, 1H), 6.26 (s, 1H), 6.38 (d, J = 6.8 Hz, 1H), 7.07 (d, J = 5.6 Hz, 1H), 7.30 (t, J = 6.8 Hz, 1H), 7.34 (s, 1H), 8.27 (d, J = 6.0 Hz, 1H), 9.98 (bs, 1H), 11.05 (bs, 1H), 11.42 (bs, 1H); ESI-MS m/z 324.2 (MH⁺). IC50 (uM): 0.201 |
| 263 | 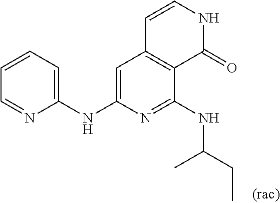 | ESI-MS m/z 310.2 (MH⁺). IC50 (uM): 0.756 |
| 264 | 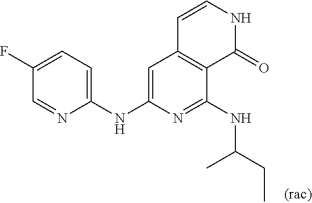 | ESI-MS m/z 328.2 (MH⁺). IC50 (uM): 0.423 |
| 265 | 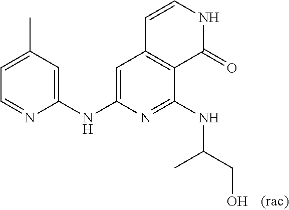 | ¹H NMR (MeOD-d₄) δ 1.34 (d, J = 6.4 Hz, 3H), 2.53 (s, 3H), 3.61 (t, J = 2.8 Hz, 1H), 3.62 (s, 1H), 4.13 (m, 1H), 6.18 (s, 1H), 6.38 (d, J = 6.8 Hz, 1H), 7.08 (s, 1H), 7.13 (dd, J = 6.4 and 1.6 Hz, 1H), 7.26 (d, J = 7.2 Hz, 1H), 8.13 (d, J = 6.4 Hz, 1H); ESI-MS m/z 326.2 (MH⁺). IC50 (uM): 0.018 |
| 266 | 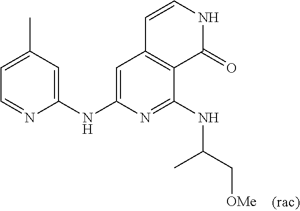 | ¹H NMR (DMSO-d₆) δ 1.92 (m, 2H), 2.42 (s, 3H), 3.24 (s, 3H), 3.46 (d, J = 6.0 Hz, 2H), 3.60 (dd, J = 12.4 and 6.4 Hz, 2H), 6.29 (s, 1H), 6.35 (d, J = 6.8 Hz, 1H), 7.06 (d, 5.6 Hz, 1H), 7.28 (t, J = 6.4 Hz, 1H), 7.36 (s, 1H), 8.21 (d, J = 6.0 Hz, 1H), 9.95 (bs, 1H), 11.36 (bs, 1H); ESI-MS m/z 340.2 (MH⁺). IC50 (uM): 0.074 |
| 267 | 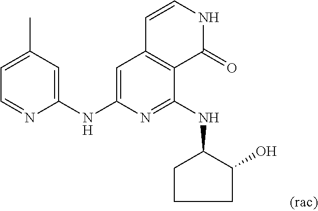 | ESI-MS m/z 352.2 (MH⁺). IC50 (uM): 0.056 |
| 268 | 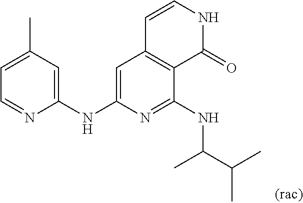 | ESI-MS m/z 338.2 (MH⁺). IC50 (uM): 0.343 |
| 269 | 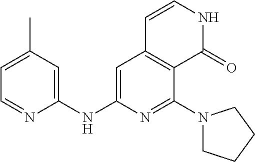 | ESI-MS m/z 322.2 (MH⁺). IC50 (uM): 0.554 |
| 270 | 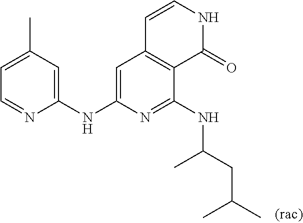 | ESI-MS m/z 352.2 (MH⁺). IC50 (uM): 6.95 |
| 271 | 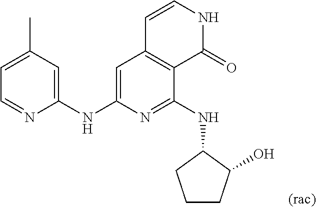 | ESI-MS m/z 352.2 (MH⁺). IC50 (uM): 0.026 |
| 272 | 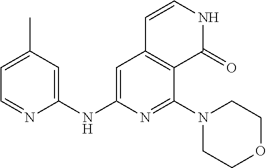 | ESI-MS m/z 338.2 (MH⁺). IC50 (uM): 0.278 |
| 273 | 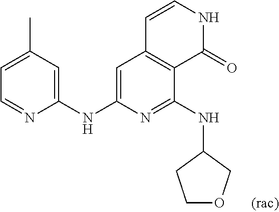 | ESI-MS m/z 338.2 (MH⁺). IC50 (uM): 0.042 |
| 274 | 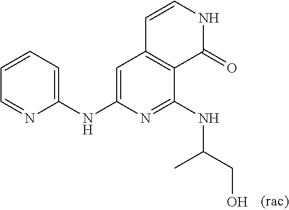 | ESI-MS m/z 312.2 (MH⁺). IC50 (uM): 0.055 |
| 275 | 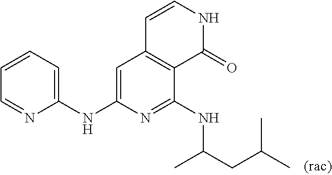 | ESI-MS m/z 338.2 (MH⁺). IC50 (uM): 3.196 |
| 276 | 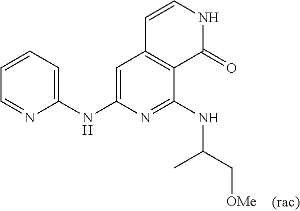 | ESI-MS m/z 326.2 (MH⁺). IC50 (uM): 0.167 |
| 277 | 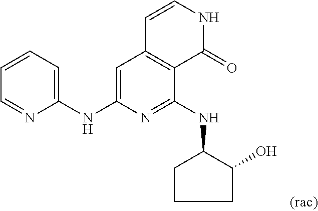 | ESI-MS m/z 338.2 (MH⁺). IC50 (uM): 0.86 |
| 278 | 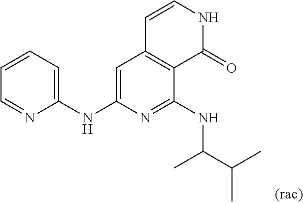 | ESI-MS m/z 324.2 (MH⁺). IC50 (uM): 2.21 |
| 279 | 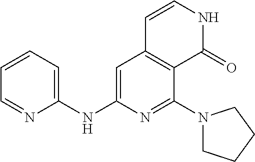 | ESI-MS m/z 308.2 (MH⁺). IC50 (uM): 1.42 |
| 280 | 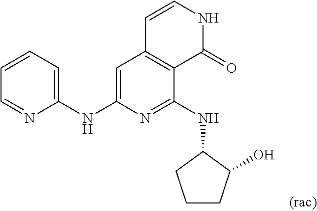 | ESI-MS m/z 338.2 (MH⁺). IC50 (uM): 0.062 |
| 281 | 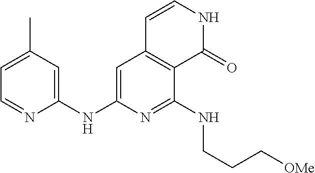 | ESI-MS m/z 340.2 (MH⁺). IC50 (uM): 0.087 |
| 282 | 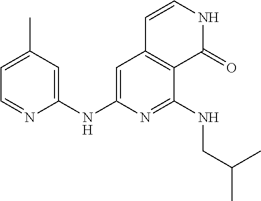 | ¹H NMR (MeOD-d₄) δ 1.00 (d, J = 6.8 Hz, 6H), 2.01 (m, 1H), 2.37 (s, 3H), 3.31 (d, J = 6.8 Hz, 2H), 6.04 (s, 1H), 6.27 (d, J = 7.2 Hz, 1H), 6.93 (s, 1H), 7.00 (dd, 6.0 and 1.2 Hz, 1H), 7.18 (d, J = 7.2 Hz, 1H), 8.03 (d, J = 6.0 Hz, 1H); ESI-MS m/z 324.2 (MH⁺). IC50 (uM): 0.50 |
| 283 | 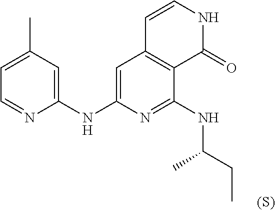 | ESI-MS m/z 324.2 (MH⁺). IC50 (uM): 0.443 |
| 284 | 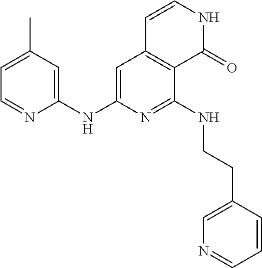 | ESI-MS m/z 373.2 (MH⁺). IC50 (uM): 0.166 |
| 285 | 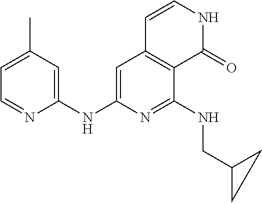 | ¹H NMR (MeOD-d₄) δ 0.45 (dt, J = 6.0 and 4.8 Hz, 2H), 0.68 (m, 2H), 1.32 (m, 1H), 2.51 (s, 3H), 3.50 (d, J = 6.8 Hz, 2H), 6.18 (s, 1H), 6.39 (d, J = 6.8 Hz, 1H), 7.07 (s, 1H), 7.13 (d, 6.0 Hz, 1H), 7.30 (d, J = 7.2 Hz, 1H), 8.18 (d, J = 6.0 Hz, 1H); ESI-MS m/z 322.2 (MH⁺). IC50 (uM): 0.201 |
| 286 | 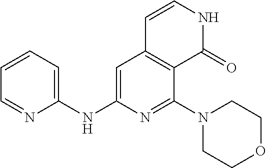 | ESI-MS m/z 324.2 (MH⁺). IC50 (uM): 0.951 |
| 287 | 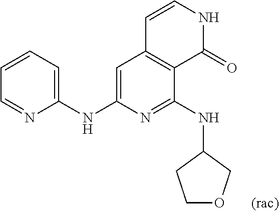 | ESI-MS m/z 324.2 (MH⁺). IC50 (uM): 0.148 |
| 288 | 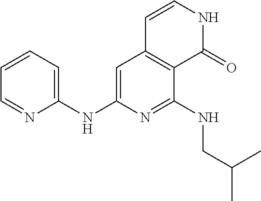 | ¹H NMR (DMSO-d₄) δ 1.01 (d, J = 6.4 Hz, 3H), 2.03 (m, 1H), 3.38 (t, J = 6.4 Hz, 3H), 6.36 (s, 1H), 6.37 (d, J = 6.8 Hz, 1H), 7.15 (t, J = 6.0 Hz, 1H), 7.30 (t, J = 6.4 Hz, 1H), 7.53 (d, J = 8.0 Hz, 1H), 7.97 (t, J = 7.6 Hz, 1H), 8.33 (dd, J = 5.2 and 1.2 Hz, 1H), 10.12 (s, 1H), 10.90 (s, 1H), 11.40 (bs, 1H); ESI-MS m/z 310.2 (MH⁺). IC50 (uM): 0.694 |
| 289 | 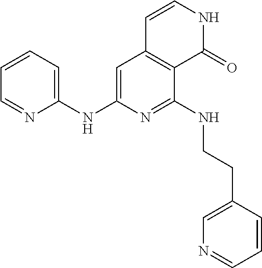 | ¹H NMR (DMSO-d₆) δ 3.13 (t, J = 6.8 Hz, 2H), 3.86 (dd, J = 12.8 and 6.8 Hz, 2H), 6.30 (d, J = 6.8 Hz, 1H), 6.50 (s, 1H), 7.10 (t, J = 6.0 Hz, 1H), 7.22 (t, J = 6.8 Hz, 1H), 7.62 (d, J = 8.4 Hz, 1H), 7.68 (dd, J = 8.0 and 5.6 Hz, 1H), 7.90 (t, J = 6.0 Hz, 1H), 8.13 (d, J = 8.0 Hz, 1H), 8.31 (d, J = 5.6 and 1.2 Hz, 1H), 8.63 (dd, J =5.2 and 1.2 Hz, 1H), 8.69 (s, 1H), 9.78(s, 1H), 10.50 (bs, 1H), 11.31 (bs, 1H), 11.25 (s, 1H); ESI-MS m/z 359.2 (MH⁺). IC50 (uM): 0.228 |
| 290 | 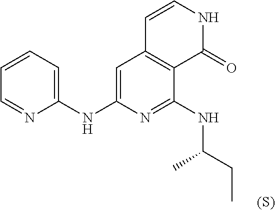 | ¹H NMR (DMSO-d₆) δ 0.97 (t, J = 10.0 and 5.2 Hz, 2H), 1.29 (d, J = 6.4 Hz, 3H), 1.68 (m, 2H), 4.01 (m, 1H), 6.33 (d, J = 6.4 Hz, 1H), 6.38 (s, 1H), 7.09 (s, 1H), 7.26 (s, 1H), 7.59 (s, 1H), 7.92 (s, 1H), 8.34 (d, J = 4.4 Hz, 1H), 9.90 (bs, 1H), 11.31 (bs, 1H); ESI-MS m/z 310.2 (MH⁺). IC50 (uM): 0.271 |
| 291 | 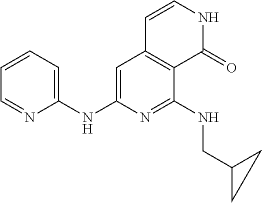 | ¹H NMR (DMSO-d₆) δ 0.30 (dd, J = 7.6 Hz, 3H), 0.49 (m, 2H), 1.18 (m, 1H), 3.38 (dd, J = 6.8 and 5.6 Hz, 2H), 6.30 (s, 1H), 6.33 (d, J = 6.8 Hz, 1H), 7.13 (t, J = 6.0 Hz, 1H), 7.27 (t, J = 6.4 Hz, 1H), 7.41 (d, J = 8.4 Hz, 1H), 7.97 (t, J = 7.2Hz, 1H), 8.29 (dd, J = 5.6 and 0.8 Hz, 1H), 10.02 (s, 1H), 11.43 (s, 1H); ESI-MS m/z 308.2 (MH⁺). IC50 (uM): 0.312 |
| 292 | 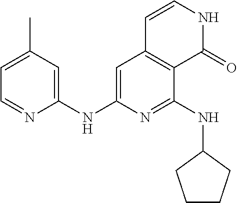 | ESI-MS m/z 336.2 (MH⁺). IC50 (uM): 0.176 |
| 293 | 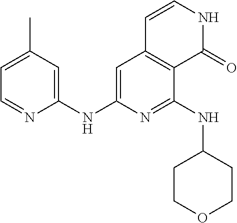 | ESI-MS m/z 352.2 (MH⁺). IC50 (uM): 0.145 |
| 294 | 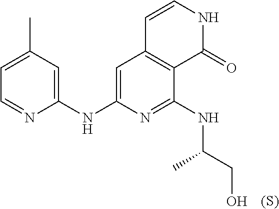 | ESI-MS m/z 326.2 (MH⁺). IC50 (uM): 0.101 |
| 295 | 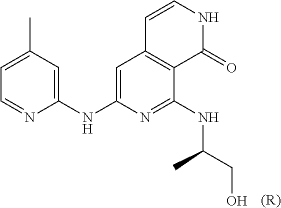 | ESI-MS m/z 326.2 (MH⁺). IC50 (uM): 0.02 |
| 296 | 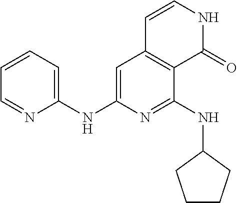 | ESI-MS m/z 322.2 (MH⁺). IC50 (uM): 0.153 |
| 297 | 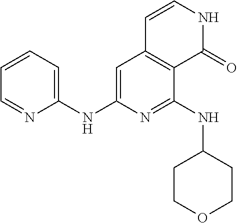 | ESI-MS m/z 338.2 (MH⁺). IC50 (uM): 0.335 |
| 298 | 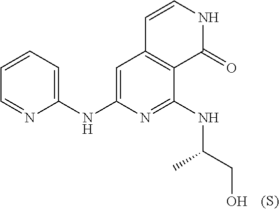 | ESI-MS m/z 312.2 (MH⁺). IC50 (uM): 0.155 |
| 299 | 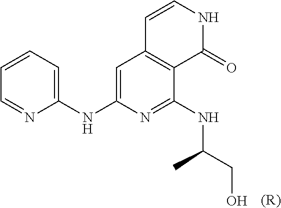 | ¹H NMR (DMSO-d₆) δ 1.29 (d, J = 6.8 Hz, 3H), 3.60 (m, 2H), 4.12 (m, 1H), 6.31 (s, 1H), 6.39 (d, J = 6.8 Hz, 1H), 7.20 (t, 6.4 Hz, 1H), 7.32 (t, J = 6.8 Hz, 1H), 7.44 (d, J = 8.0 Hz, 1H), 8.04 (t, J =7.6 Hz, 1H), 8.34 (dd, J = 5.6 and 1.2 Hz, 1H), 10.05 (s, 1H), 11.15 (bs, 1H), 11.45 (s, 1H); ESI-MS m/z 312.2 (MH⁺). IC50 (uM): 0.068 |
| 300 | 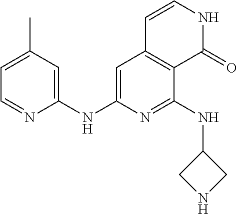 | ESI-MS m/z 323.2 (MH⁺). IC50 (uM): 1.544 |
| 301 | 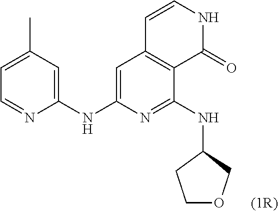 | ESI-MS m/z 338.2 (MH⁺). IC50 (uM): 0.037 |
| 302 | 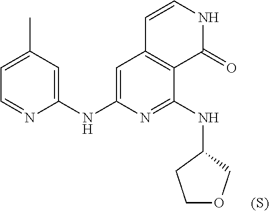 | ESI-MS m/z 338.2 (MH⁺). IC50 (uM): 0.083 |
| 303 | 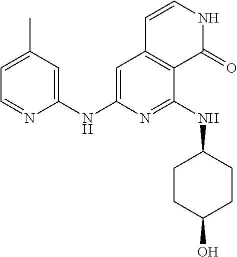 | ESI-MS m/z 366.2 (MH⁺). IC50 (uM): 0.017 |
| 304 | 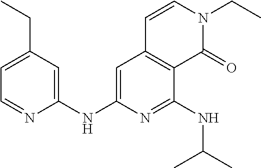 | ¹H NMR (DMSO-d₆) δ 1.24 (t, J = 7.6 Hz, 6H), 1.36 (d, J = 6.4 Hz, 6H), 2.75 (q, J = 6.8 Hz, 2H), 3.93 (q, J = 6.8 Hz, 2H), 4.15 (m, 1H), 6.29 (s, 1H), 6.51 (d, J = 7.2 Hz, 1H), 7.18 (d, J = 6.0 Hz, 1H), 7.34 (s, 1H), 7.68 (d, J = 7.2 Hz, 1H), 8.34 (d, J = 6.0 Hz, 1H), 10.07 (d, J =6.8 Hz, 1H), 11.66 (bs, 1H); ESI-MS m/z 352.2 (MH⁺). IC50 (uM): 0.384 |
| 305 | 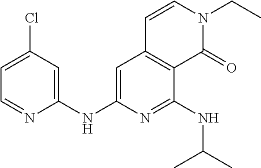 | ESI-MS m/z 358.2 (MH⁺). IC50 (uM): 0.257 |
| 306 | 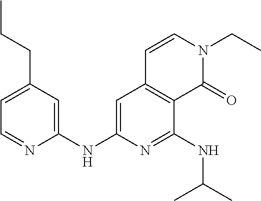 | ESI-MS m/z 366.2 (MH⁺). IC50 (uM): 0.612 |
| 307 | 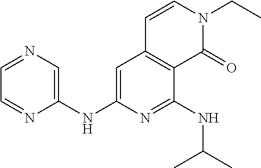 | ¹H NMR (DMSO-d₆) δ 1.23 (t, J = 7.2 Hz, 3H), 1.32 (d, J = 6.4 Hz, 6H), 3.90 (q, J = 6.8 Hz, 2H), 4.22 (m, 1H), 6.40 (d, J = 6.8 Hz, 1H), 6.64 (s, 1H), 7.59 (d, J = 6.0 Hz, 1H), 8.19 (d, J = 1.2 Hz, 1H), 8.32 (s, 1H), 9.04 (s, 1H), 9.91 (bs, 1H); ESI-MS m/z 325.2 (MH⁺). IC50 (uM): 0.06 |
| 308 | 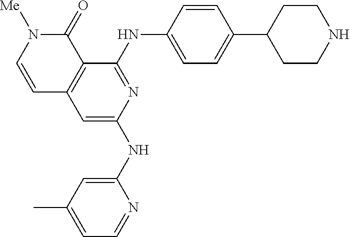 | ESI-MS m/z 441.2 (MH⁺). IC50 (uM): 0.007 |
| 309 | 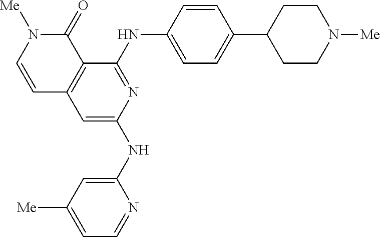 | ¹H NMR (DMSO-d₆) δ 2.04 (m, 4H), 2.38 (s, 3H), 2.79 (d, J = 4.8 Hz, 6H), 2.88 (m, 1H), 3.08 (m, 2H), 3.50 (s, 3H), 3.52 (s, 2H), 6.53 (d, J = 7.2 Hz, 1H), 6.67 (s, 1H), 7.05 (d, J = 5.2 Hz, 1H), 7.34 (d, J = 8.4 Hz, 2H), 7.62 (d, J = 8.0 Hz, 2H), 7.67 (d, J = 7.2 Hz, 2H), 10.49 (bs, 1H), 11.93 (s, 1H); ESI-MS m/z 455.2 (MH⁺). IC50 (uM): 0.016 |
| 310 | 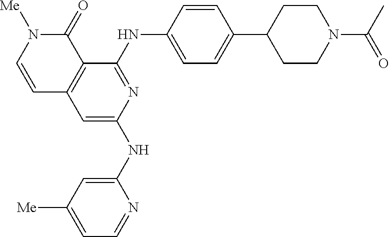 | ESI-MS m/z 483.2 (MH⁺). IC50 (uM): 0.763 |
| 311 | 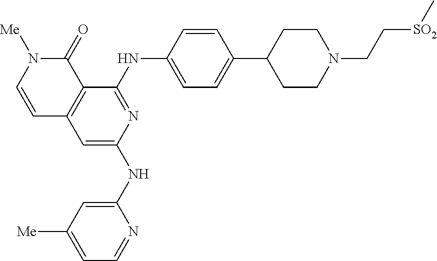 | ESI-MS m/z 547.2 (MH⁺). IC50 (uM): 0.496 |
| 312 | 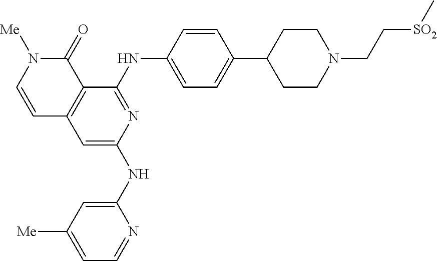 | ESI-MS m/z 494.2 (MH⁺). IC50 (uM): 0.505 |
| 313 | 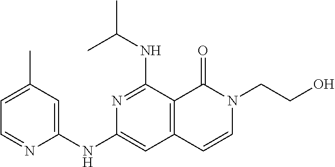 | ¹H NMR (DMSO-d₆) δ 1.36 (d, J = 6.4 Hz, 6H), 2.45 (s, 3H), 3.65 (t, J = 5.6 Hz, 2H), 3.96 (t, J = 5.2 Hz, 2H), 4.15 (m, 1H), 6.28 (s, 1H), 6.46 (d, J = 7.2 Hz, 1H), 7.13 (d, J = 6.0 Hz, 1H), 7.30 (s, 1H), 7.56 (d, J = 7.6 Hz, 1H), 8.32 (d, J = 6.0 Hz, 1H), 10.05 (d, J = 6.4 Hz, 1H), 11.52 (bs, 1H); ESI-MS m/z 354.2 (MH⁺). IC50 (uM): 0.085 |
| 314 | 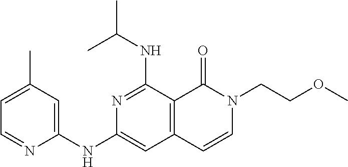 | ¹H NMR (MeOD-d₄) δ 1.48 (d, J = 6.8 Hz, 6H), 2.50 (s, 3H), 3.31 (m, 4H), 3.68 (t, J = 5.2 Hz, 1H), 4.14 (t, J = 4.8 Hz, 1H), 4.17 (m, 1H), 6.17 (s, 1H), 6.39 (d, J = 7.2 Hz, 1H), 7.07 (s, 1H), 7.12 (d, J = 6.0 Hz, 1H), 7.49 (d, J = 7.6 Hz, 1H), 8.21 (d, J = 6.0 Hz, 1H); ESI-MS m/z 368.2 (MH⁺). IC50 (uM): 0.237 |
| 315 | 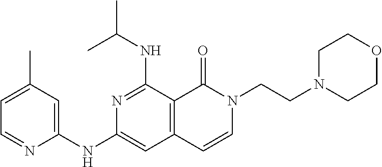 | ESI-MS m/z 423.2 (MH⁺). IC50 (uM): 0.381 |
| 316 | 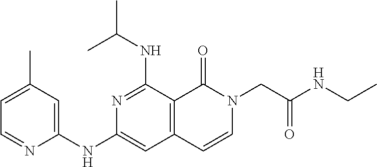 | ¹H NMR (MeOD-d₄) δ 1.17 (t, J =7.2 Hz, 3H), 1.46 (d, J = 6.4 Hz, 6H), 2.51 (s, 3H), 3.31 (m, 4H), 4.16 (m, 1H), 4.60 (s, 2H), 6.18 (s, 1H), 6.42 (d, J = 7.6 Hz, 1H), 7.07 (s, 1H), 7.13 (dd, J = 6.0 and 1.2 Hz, 1H), 7.47 (d, J = 7.6 Hz, 1H), 8.21 (d, J = 6.4 Hz, 1H); ESI-MS m/z 395.2 (MH⁺). IC50(uM): 0.27 |
| 317 | 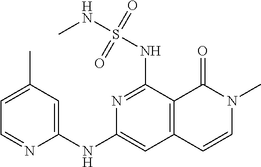 | ¹H NMR (DMSO-d₆) δ 2.32 (s, 3H), 2.42 (d, J = 5.2 Hz, 3H), 3.50 (s, 3H), 6.51 (d, J = 7.6 Hz, 1H), 6.92 (d, J = 5.2 Hz, 1H), 7.20 (s, 1H), 7.32 (s, 1H), 7.36 (q, J = 4.8 Hz, 1H), 7.60 (d, J = 7.2 Hz, 1H), 7.72 (d, J = 8.8 Hz, 1H), 8.04 (d, J = 8.4 Hz, 2H), 8.09 (d, J = 5.6 Hz, 1H), 10.13 (bs, 1H), 12.46 (s, 1H); ESI-MS m/z 451.2 (MH⁺). IC50(uM): 0.218 |
| 318 | 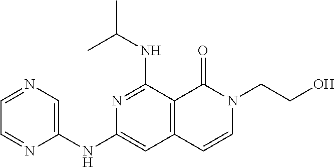 | ¹H NMR (DMSO-d₆) δ 1.28 (d, J = 6.4 Hz, 6H), 3.65 (t, J = 5.6 Hz, 2H), 3.91 (t, J = 5.6 Hz, 2H), 4.26 (m, 1H), 6.27 (d, J = 7.2 Hz,1H), 6.63 (s, 1H), 7.38 (d, J = 7.2 Hz, 1H), 8.13 (d, J = 2.8 Hz, 1H), 8.28 (dd, J = 2.8 and 1.6 Hz, 1H), 9.18 (s, 1H), 9.59 (s, 1H), 9.99 (bs, 1H); ESI-MS m/z 341.2 (MH⁺). IC50 (uM): 0.052 |
| 319 | 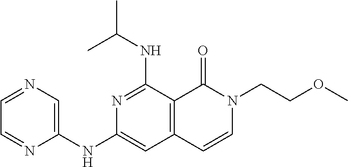 | ESI-MS m/z 355.2 (MH⁺). IC50 (uM): 0.137 |
| 320 | 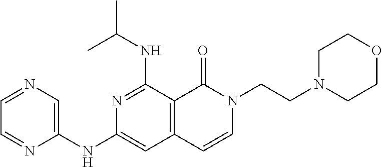 | ESI-MS m/z 410.2 (MH⁺). IC50 (uM): 0.20 |
| 321 | 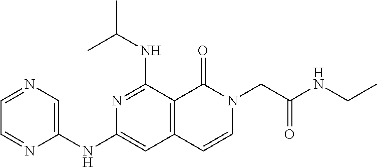 | ESI-MS m/z 382.2 (MH⁺). IC50 (uM): 0.177 |
| 322 | 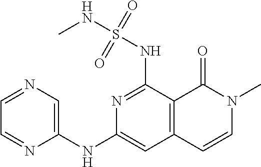 | ESI-MS m/z 438.2 (MH⁺). IC50 (uM): 0.803 |
| 323 | 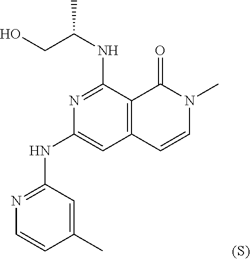 | ESI-MS m/z 340.2 (MH⁺). IC50 (uM): 0.037 |
| 324 | 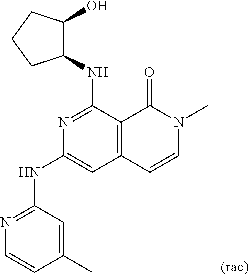 | ESI-MS m/z 366.2 (MH⁺). IC50 (uM): 0.098 |
| 325 | 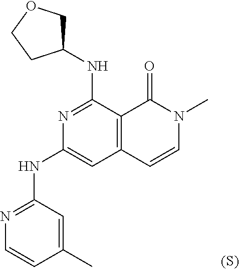 | ¹H NMR (DMSO-d₆) δ 1.98 (m, 1H), 2.37 (m, 1H), 2.47 (s, 3H), 3.44 (s, 3H), 3.85 (m, 4H), 4.67 (s, 1H), 6.36 (s, 1H), 6.48 (d, J = 7.2 Hz, 1H), 7.17 (d, J = 6.0 Hz, 1H), 7.38 (s, 1H), 7.65 (d, J = 7.2 Hz, 1H), 8.36 (d, J = 6.4 Hz, 1H), 10.16 (d, J = 6.4 Hz, 1H), 11.93 (bs, 1H); ESI-MS m/z 352.2 (MH⁺). IC50 (uM): 0.057 |
| 326 | 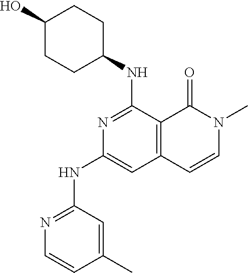 | ESI-MS m/z 380.2 (MH⁺). IC50 (uM): 0.032 |
| 327 | 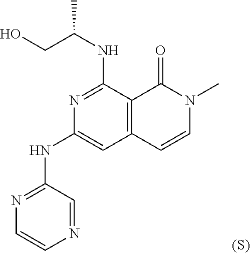 | ¹H NMR (DMSO-d₆) δ 1.23 (t, J = 7.2 Hz, 3H), 1.28 (d, J = 6.4 Hz, 3H), 3.58 (m, 2H), 3.91 (q, J = 7.2 Hz, 2H), 4.18 (bs, 1H), 6.43 (d, J = 6.8 Hz, 1H), 6.66 (s, 1H), 7.62 (d, J = 6.4 Hz, 1H), 8.21 (d, J = 2.4 Hz, 1H), 8.32 (s, 1H), 8.96 (s, 1H), 10.15 (bs, 1H); ESI-MS m/z 327.2 (MH⁺). ESI-MS m/z 327.2 (MH⁺). IC50 (uM): 0.12 |
| 328 | 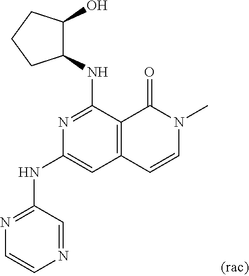 | ¹H NMR (DMSO-d₆) δ 1.64 (m, 4H), 1.84 (m, 2H), 2.15 (s, 1H), 3.42 (s, 3H), 4.18 (s, 2H), 6.38 (s, 1H), 6.62 (s, 1H), 7.58 (s, 1H), 8.19 (s, 1H), 8.29 (s, 1H), 8.99 (s, 1H), 10.25 (bs, 1H); ESI-MS m/z 353.2 (MH⁺). IC50 (uM): 0.039 |
| 329 | 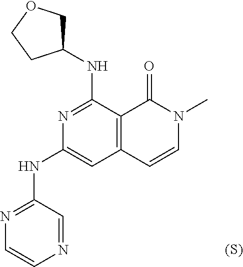 | ESI-MS m/z 339.2 (MH⁺). IC50 (uM): 0.063 |
| 330 | 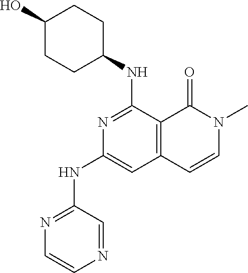 | ESI-MS m/z 367.2 (MH⁺). IC50 (uM): 0.024 |
| 331 | 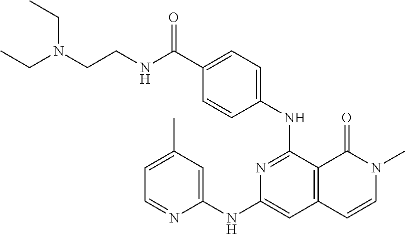 | ESI-MS m/z 500.2 (MH⁺). IC50 (uM): 0.748 |
| 332 | 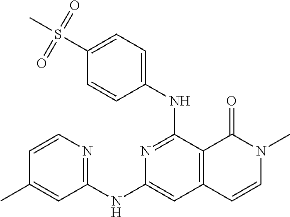 | ¹H NMR (DMSO-d₆) δ 2.36 (s, 3H), 3.22 (s, 3H), 3.51 (s, 3H), 6.55 (d, J = 6.8 Hz, 3H), 6.98 (s, 1H), 7.14 (bs, 1H), 7.33 (s, 1H), 7.64 (d, J = 6.8 Hz, 1H), 7.87 (d, J = 8.4 Hz, 2H), 8.07 (m, 3H), 12.53 (s, 1H); ESI-MS m/z 436.2 (MH⁺). IC50 (uM): 0.078 |
| 333 | 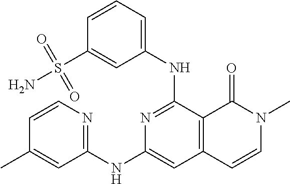 | ESI-MS m/z 433.2 (MH⁺). IC50 (uM): 0.294 |
| 334 | 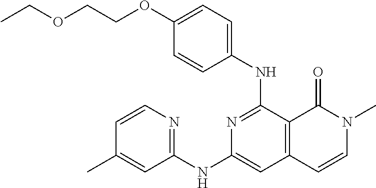 | ESI-MS m/z 446.2 (MH⁺). IC50 (uM): 1.715 |
| 335 | 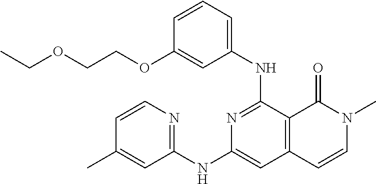 | ESI-MS m/z 446.2 (MH⁺). IC50 (uM): 1.606 |
| 336 | 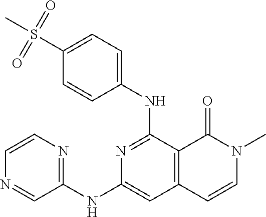 | ¹H NMR (MeOD-d₄) δ 3.15 (s, 3H), 3.51 (s, 3H), 6.41 (d, J = 6.4 Hz, 1H), 7.36 (s, 1H), 7.38 (s, 1H), 7.87 (s, 1H), 7.89 (s, 1H), 8.07 (m, 3H), 8.22 (s, 1H), 8.58 (s, 1H); ESI-MS m/z 423.2 (MH⁺). IC50 (uM): 0.584 |
| 337 | 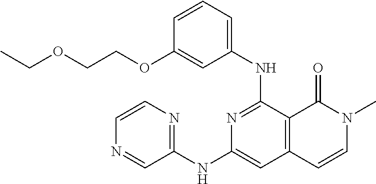 | ESI-MS m/z 433.2 (MH⁺). IC50 (uM): 2.13 |
| 338 | 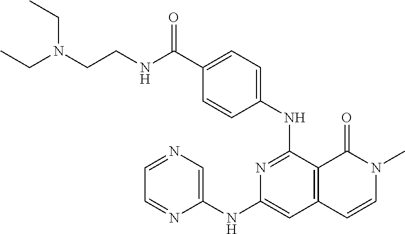 | ESI-MS m/z 487.2 (MH⁺). IC50 (uM): 1.615 |
| 339 | 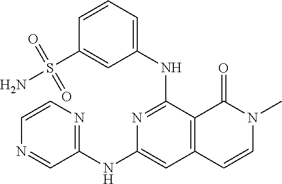 | ESI-MS m/z 424.2 (MH⁺). IC50 (uM): 0.879 |
| 340 | 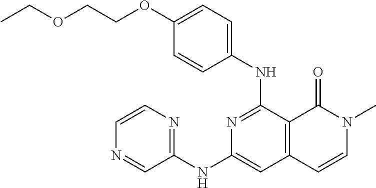 | ESI-MS m/z 433.2 (MH⁺). IC50 (uM): 2.06 |
| 341 | 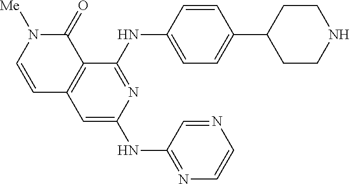 | ESI-MS m/z 428.2 (MH⁺). IC50 (uM): 0.112 |
| 342 | 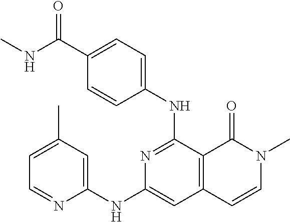 | ESI-MS m/z 415.2 (MH⁺). IC50 (uM): 0.222 |
| 343 | 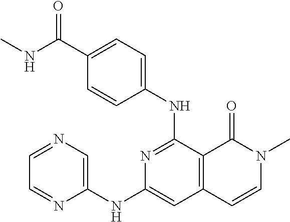 | ESI-MS m/z 402.2 (MH⁺). IC50 (uM): 0.333 |
| 344 | 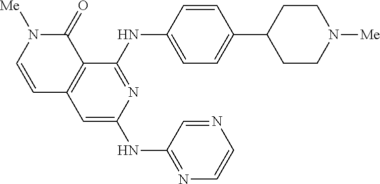 | ESI-MS m/z 442.2 (MH⁺). IC50 (uM): 0.045 |
| 345 | 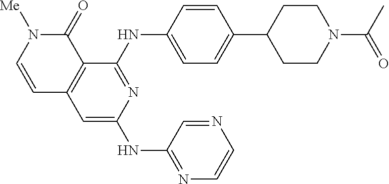 | ¹H NMR (DMSO-d₆) δ 1.48 (m, 1H), l.59 (m, 1H), 1.81 (t, J = 13.2 Hz, 2H), 2.59 (m, 1H), 2.77 (m, 1H), 3.14 (m, 1H), 3.48 (s, 3H), 3.93 (d, J = 12.0 Hz, 1H), 4.54 (d, J = 11.2 Hz, 1H), 6.45 (d, J = 7.6 Hz, 1H), 7.20 (s, 1H), 7.23 (d, J = 8.4 Hz, 2H), 7.57 (d, J = 7.2 Hz, 1H), 7.74 (d, J = 8.8 Hz, 2H), 8.14 (d, J = 2.4 Hz, 1H), 8.25 (s, 1H), 8.83 (d, J = 1.2 Hz, 1H), 10.11 (s, 1H), 12.04 (s, 1H); ESI-MS m/z 470.2 (MH⁺). IC50 (uM): 1.222 |
| 346 | 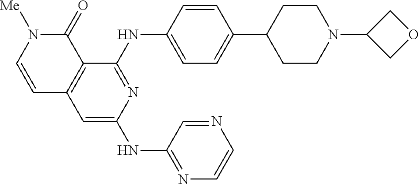 | ESI-MS m/z 484.2 (MH⁺). IC50 (uM): 0.387 |
| 347 | 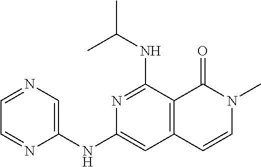 | ESI-MS m/z 311.2 (MH⁺). IC50 (uM): 0.057 |
| 348 | 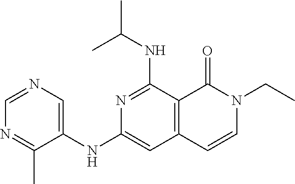 | ESI-MS m/z 339.2 (MH⁺). IC50 (uM): 7.44 |
| 349 | 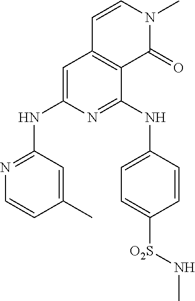 | ESI-MS m/z 451.2 (MH⁺). IC50 (uM): 0.414 |
| 350 | 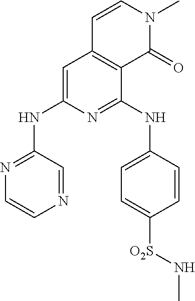 | ESI-MS m/z 438.2 (MH⁺). IC50 (uM): 0.803 |
| 351 | 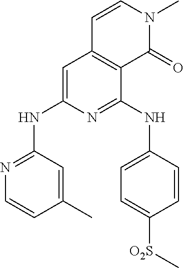 | ESI-MS m/z 455.2 (MH⁺). IC50 (uM): 0.078 |
| 352 | 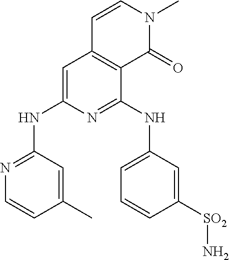 | ESI-MS m/z 437.2 (MH⁺). IC50 (uM): 0.294 |
| 353 | 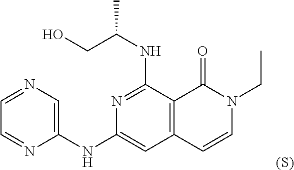 | ESI-MS m/z 341.2 (MH⁺). IC50 (uM): 0.035 |
| 354 | 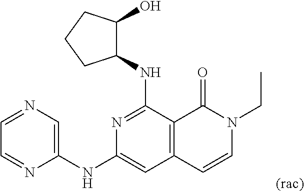 | ESI-MS m/z 367.2 (MH⁺). IC50 (uM): 0.018 |
| 355 | 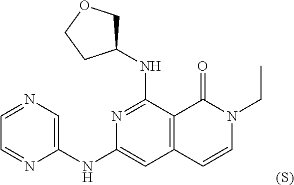 | ESI-MS m/z 353.2 (MH⁺). IC50 (uM): 0.037 |
| 356 | 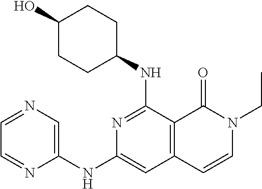 | ESI-MS m/z 381.2 (MH⁺). IC50 (uM): 0.010 |
| 357 | 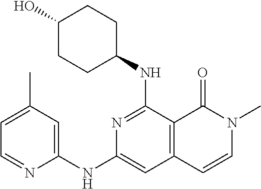 | ESI-MS m/z 380.2 (MH⁺). IC50 (uM): 0.041 |
| 358 | 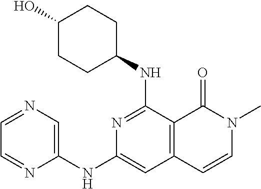 | ¹H NMR (DMSO-d₆) δ 1.31 (m, 4H), 1.81 (m, 2H), 2.03 (m, 2H), 3.33 (s, 3H), 3.45 (m, 1H), 3.82 (s, 1H), 6.28 (d, J = 7.2 Hz, 1H), 6.50 (s, 1H), 7.47 (d, J = 6.8 Hz, 1H), 8.12 (d, J = 2.4 Hz, 1H), 8.22 (dd, J = 2.8 and 1.6 Hz, 1H), 9.08 (s, 1H), 9.71 (bs, 1H); ESI-MS m/z 367.2 (MH⁺). IC50 (uM): 0.049 |
| 359 | 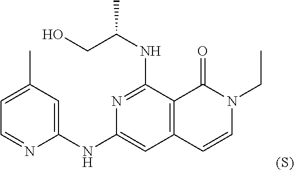 | ESI-MS m/z 354.2 (MH⁺). IC50 (uM): 0.025 |
| 360 | 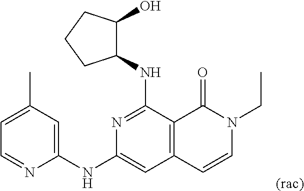 | ESI-MS m/z 380.2 (MH⁺). IC50 (uM): 0.067 |
| 361 | 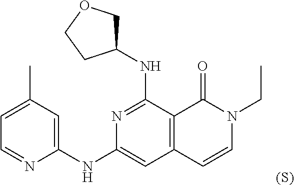 | ESI-MS m/z 366.2 (MH⁺). IC50 (uM): 0.03 |
| 362 | 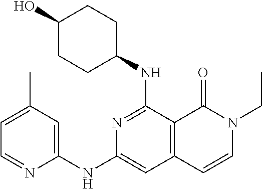 | ESI-MS m/z 394.2 (MH⁺). IC50 (uM): 0.018 |
| 363 | 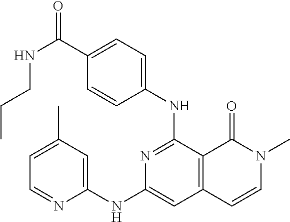 | ESI-MS m/z 443.2 (MH⁺). IC50 (uM): 0.551 |
| 364 | 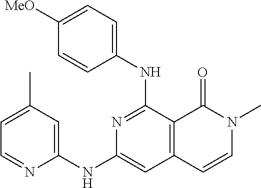 | ¹H NMR (DMSO-d₆) δ 3.40 (s, 3H), 3.71 (s, 3H), 6.37 (d, J = 7.2 Hz, 6H), 6.88 (d, J = 8.8 Hz, 2H), 7.02 (s, 1H), 7.50 (d, J = 7.2 Hz, 1H), 7.62 (d, J = 8.8 Hz, 1H), 8.05 (d, J = 2.8 Hz, 1H), 8.14 (s, 1H), 8.76 (d, J = 1.6 Hz, 1H), 10.03 (s, 1H), 11.80 (s, 1H); ESI-MS m/z 388.2 (MH⁺). IC50 (uM): 0.228 |
| 365 | 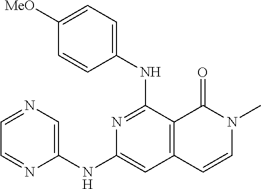 | ESI-MS m/z 375.2 (MH⁺). IC50 (uM): 0.361 |
| 366 | 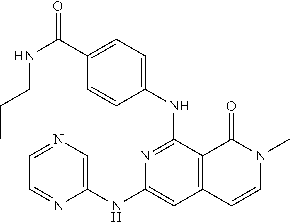 | ESI-MS m/z 430.2 (MH⁺). IC50 (uM): 0.323 |
| 367 | 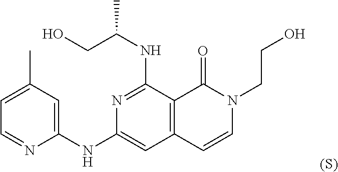 | ESI-MS m/z 370.2 (MH⁺). IC50 (uM): 0.013 |
| 368 | 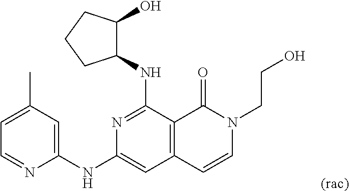 | ESI-MS m/z 396.2 (MH⁺). IC50 (uM): 0.016 |
| 369 | 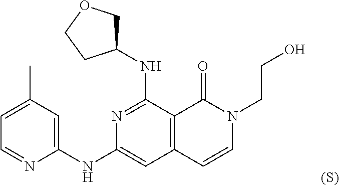 | ESI-MS m/z 382.2 (MH⁺). IC50 (uM): 0.012 |
| 370 | 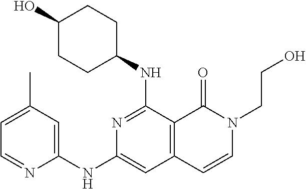 | ESI-MS m/z 410.2 (MH⁺). IC50 (uM): 0.026 |
| 371 | 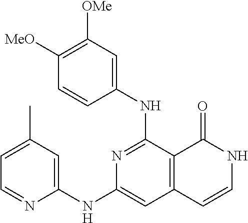 | ESI-MS m/z 404.2 (MH⁺). IC50 (uM): 0.233 |
| 372 | 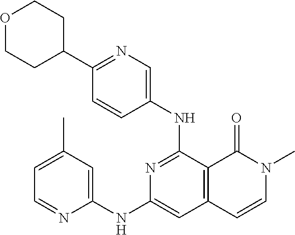 | ¹H NMR (MeOD-d₄) δ 1.95 (m, 4H), 2.52 (s, 3H), 3.21 (m, 1H), 3.60 (s, 3H), 3.64 (m, 2H), 4.12 (d, J = 10.8 Hz, 2H), 6.53 (d, J = 6.8 Hz, 1H), 6.60 (s, 1H), 7.15 (d, J = 6.0 Hz, 1H), 7.21 (s, 1H), 7.57 (d, J = 6.8 Hz, 1H), 7.65 (d, J = 6.0 Hz, 1H), 7.72 (d, J = 8.0 Hz, 1H), 8.28 (d, J = 7.6 Hz, 1H), 9.05 (s, 1H); ESI-MZ m/z 443.2 (MH⁺). IC50 (uM): 0.393 |
| 373 | 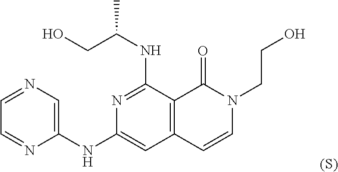 | ESI-MS m/z 357.2 (MH⁺). IC50 (uM): 0.027 |
| 374 | 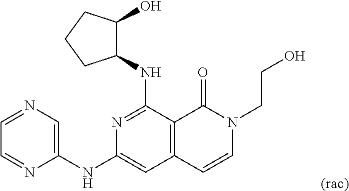 | ESI-MS m/z 433.2 (MH⁺). IC50 (uM): 0.013 |
| 375 | 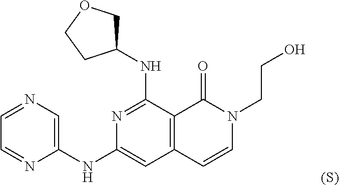 | ESI-MS m/z 369.2 (MH⁺). IC50 (uM): 0.014 |
| 376 | 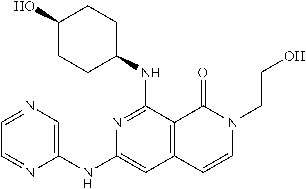 | ESI-MS m/z 397.2 (MH⁺). IC50 (uM): 0.006 |
| 377 | 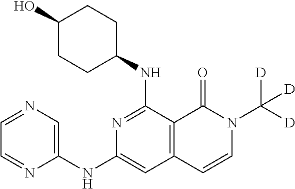 | ¹H NMR (DMSO-d₆) δ 1.59 (m, 2H), 1.68 (m, 2H), 1.78 (m, 4H), 3.69 (m, 1H), 4.07 (m, 1H), 6.38 (d, J = 6.8 Hz, 1H), 6.61 (s, 1H), 7.56 (d, J = 7.2 Hz, 1H), 8.18 (d, J = 2.4 Hz, 1H), 8.30 (s, 1H), 9.09 (s, 1H), 10.13 (bs, 1H); ESI-MS m/z 370.2 (MH⁺). IC50 (uM): 0.034 |
| 378 | 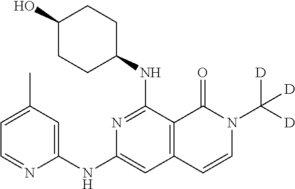 | ESI-MS m/z 383.2 (MH⁺). IC50 (uM): 0.02 |
| 379 | 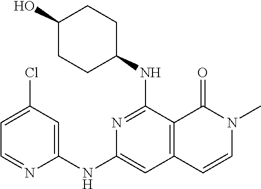 | ESI-MS m/z 400.2 (MH⁺). IC50 (uM): 0.036 |
| 380 | 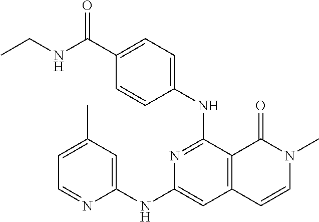 | ESI-MS m/z 429.2 (MH⁺). IC50 (uM): 0.379 |
| 381 | 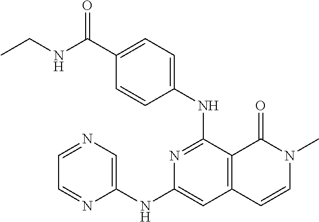 | ESI-MS m/z 416.2 (MH⁺). IC50 (uM): 0.604 |
| 382 | 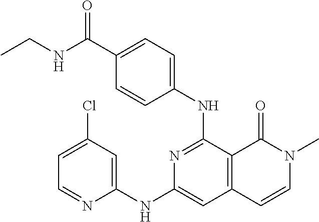 | ESI-MS m/z 449.2 (MH⁺). IC50 (uM): 0.362 |
| 383 | 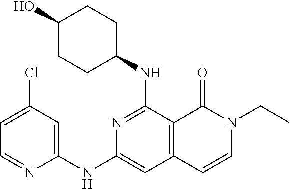 | ¹H NMR (DMSO-d₆) δ 1.36 (t, J = 7.2 Hz, 3H), 1.80 (m, 2H), 1.90 (m, 2H), 2.05 (m, 4H), 3.90 (m, 1H), 3.97 (m, 1H), 4.05 (q, J = 7.2 Hz, 2H), 6.19 (s, 1H), 6.51 (d, J = 7.2 Hz, 1H), 7.17 (d, J = 1.6 Hz, 1H), 7.25 (dd, J = 6.0 and 2.0 Hz, 1H), 7.69 (d, J = 7.2 Hz, 1H), 8.31 (d, J = 5.6 Hz, 1H); ESI-MS m/z 414.2 (MH⁺). IC50 (uM): 0.026 |
| 384 | 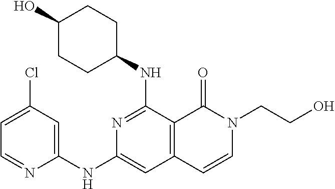 | ESI-MS m/z 430.2 (MH⁺). IC50 (uM): 0.017 |
| 385 | 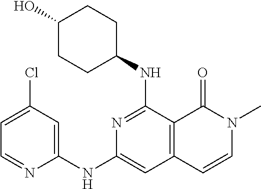 | ¹H NMR (DMSO-d₆) δ 1.63 (m, 4H), 2.11 (m, 2H), 2.32 (m, 2H), 3.55 (s, 3H), 3.80 (m, 2H), 6.20 (s, 1H), 6.49 (d, J = 7.2 Hz, 1H), 7.18 (d, J = 1.6 Hz, 1H), 7.28 (dd, J = 5.6 and 1.6 Hz, 1H), 7.66 (d, J = 7.2 Hz, 1H), 8.32 (d, J = 5.6 Hz, 1H); ESI-MS m/z 400.2 (MH⁺). IC50 (uM): 0.028 |
| 386 | 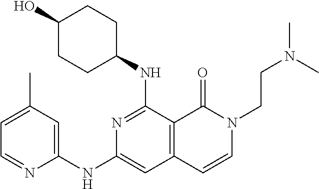 | ESI-MS m/z 437.2 (MH⁺). IC50 (uM): 0.049 |
| 387 | 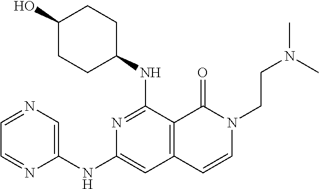 | ESI-MS m/z 424.2 (MH⁺). IC50 (uM): 0.033 |
| 388 | 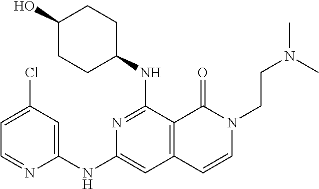 | ESI-MS m/z 457.2 (MH⁺). IC50 (uM): 0.042 |
| 389 | 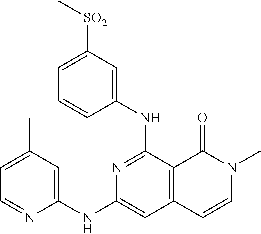 | ¹H NMR (DMSO-d₆) δ 2.52 (s, 3H), 3.16 (s, 3H), 3.60 (s, 3H), 6.48 (s, 1H), 6.53 (d, J = 7.2 Hz, 1H), 7.08 (dd, J = 6.4 and 1.6 Hz, 1H), 7.14 (s, 1H), 7.45 (d, J = 6.8 Hz, 1H), 7.58 (d, J = 7.2 Hz, 1H), 7.79 (d, J = 8.0 Hz, 1H), 7.87 (m, 2H), 8.26 (t, J = 2.0 Hz, 1H); ESI-MS m/z 400.2 (MH⁺). IC50 (uM): 0.132 |
| 390 | 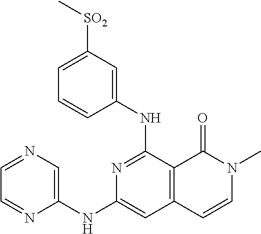 | ESI-MS m/z 423.2 (MH⁺). IC50 (uM): 0.751 |
| 391 | 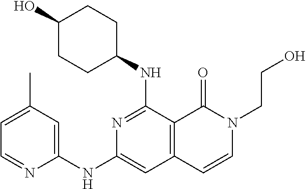 | ESI-MS m/z 410.2 (MH⁺). IC50 (uM): 0.008 |
| 392 | 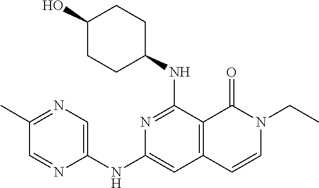 | ESI-MS m/z 395.2 (MH⁺). IC50 (uM): 0.193 |
| 393 | 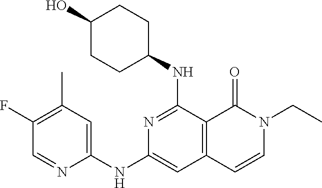 | ESI-MS m/z 412.2 (MH⁺). IC50 (uM): 0.09 |
| 394 | 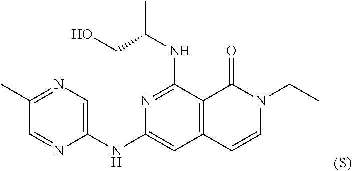 | ESI-MS m/z 355.2 (MH⁺). IC50 (uM): 0.384 |
| 395 | 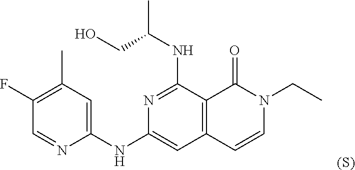 | ESI-MS m/z 372.2 (MH⁺). IC50 (uM): 0.085 |
| 396 | 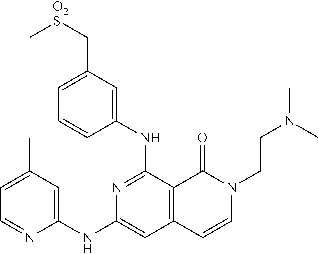 | ESI-MS m/z 493.2 (MH⁺). IC50 (uM): 0.098 |
| 397 | 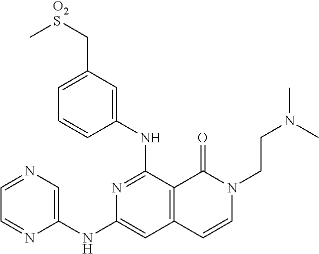 | ESI-MS m/z 480.2 (MH⁺). IC50 (uM): 0.976 |
| 398 | 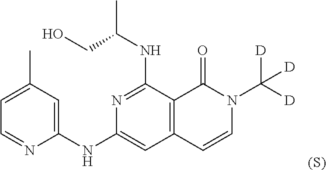 | ESI-MS m/z 343.2 (MH⁺). IC50 (uM): 0.046 |
| 399 | 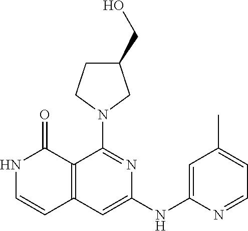 | ESI-MS m/z 352.2 (MH⁺). IC50 (uM): 0.998 |
| 400 | 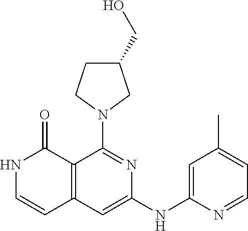 | ESI-MS m/z 352.2 (MH⁺). IC50 (uM): 0.897 |
| 401 | 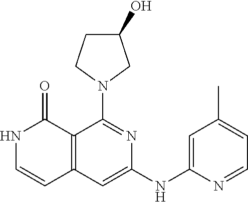 | ESI-MS m/z 338.2 (MH⁺). IC50 (uM): 0.967 |
| 402 | 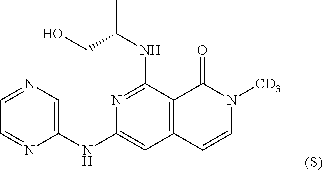 | ¹H NMR (DMSO-d₆) δ 1.19 (d, J = 6.8 Hz, 3H), 3.50 (m, 2H), 4.12 (s, 1H), 6.31 (d, J = 7.2 Hz, 1H), 6.58 (s, 1H), 7.50 (d, J = 6.8 Hz, 1H), 8.12 (d, J = 2.4 Hz, 1H), 8.24 (s, 1H), 9.95 (bs, 1H); ESI-MS m/z 330.2 (MH⁺). IC50 (uM): 0.0457 |
| 403 | 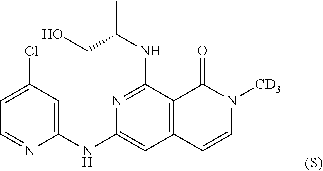 | ESI-MS m/z 363.2 (MH⁺). IC50 (uM): 0.0604 |
| 404 | 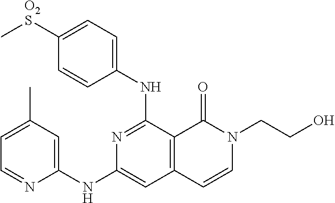 | ESI-MS m/z 466.2 (MH⁺). IC50 (uM): 0.0634 |
| 405 | 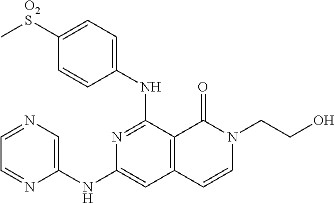 | ¹H NMR (MeOD-d₄) δ 3.23 (s, 3H), 3.89 (t, J = 5.2 Hz, 2H), 4.14 (t, J = 5.2 Hz, 2H), 6.53 (d, J = 7.6 Hz, 1H), 6.94 (s, 1H), 7.59 (d, J = 7.0 Hz, 1H), 7.96 (s, 1H), 8.00 (d, J = 8.8 Hz, 1H), 8.01 (s, 1H), 8.08 (s, 1H), 8.09 (d, J = 8.8 Hz, 1H), 8.19 (d, J = 2.8 Hz, 1H), 8.56 (s, 1H); ESI-MS m/z 453.2 (MH⁺). IC50 (uM): 0.235 |
| 406 | 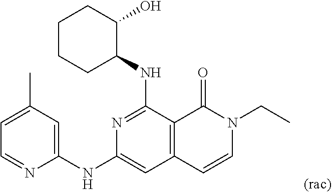 | ¹H NMR (MeOD-d₄) δ 1.34 (t, J =7.2 Hz, 3H), 1.42~1.57 (m, 4H), 1.84 (bs, 2H), 2.10 (m, 2H), 2.52 (s, 3H), 3.59 (m, 1H), 3.81 (m, 1H), 4.01 (q, J = 7.2 Hz, 2H), 6.16 (s, 1H), 6.42 (d, J = 7.6 Hz, 1H), 7.06 (s, 1H), 7.13 (dd, J = 6.4 and 1.2 Hz, 1H), 7.49 (d, J = 7.2 Hz, 1H), 8.09 (d, J = 6.0 Hz, 1H); ESI-MS m/z 394.2 (MH⁺). IC50 (uM): 0.476 |
| 407 | 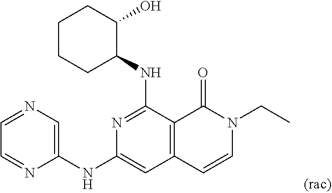 | ESI-MS m/z 381.2 (MH⁺). IC50 (uM): 0.301 |
| 408 | 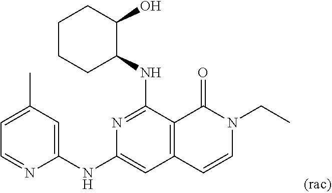 | ¹H NMR (MeOD-d₄) δ 1.35 (t, J = 7.2 Hz, 3H), 1.50~1.92 (m, 8H), 2.50 (s, 3H), 4.01 (m, 3H), 4.28 (bs, 1H), 6.15 (s, 1H), 6.43 (d, J = 7.2 Hz, 1H), 7.05 (s, 1H), 7.12 (dd, J = 6.4 and 1.2 Hz, 1H), 7.53 (d, J = 7.2 Hz, 1H), 8.14 (d, J = 6.0 Hz, 1H); ESI-MS m/z 394.2 (MH⁺). IC50 (uM): 0.324 |
| 409 | 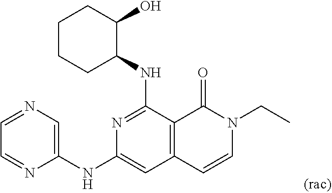 | ¹H NMR (DMSO-d₆) δ 1.23 (t, J = 7.2 Hz, 3H), 1.40 (m, 2H), 1.56-1.76 (m, 6H), 3.91 (q, J = 7.2 Hz, 2H), 3.94 (s, 1H), 4.04 (s, 1H), 6.40 (d, J = 5.6 Hz, 1H), 6.55 (s, 1H), 7.61 (s, 1H), 8.21 (s, 1H), 8.31 (s, 1H), 9.04 (bs, 1H), 10.30 (bs, 1H); ESI-MS m/z 381.2 (MH⁺). IC50 (uM): 0.128 |
| 410 | 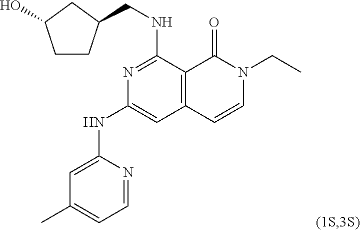 | ¹H NMR (MeOD-d₄) δ 1.34 (t, J = 7.2 Hz, 3H), 1.42~1.72 (m, 3H), 1.93~2.17 (m, 3H), 2.50 (s, 3H), 2.72 (m, 1H), 3.58 (d, J = 7.2 Hz, 2H), 4.02 (q, J = 7.2 Hz, 2H), 4.39 (m, 1H), 6.17 (s, 1H), 6.44 (d, J = 7.6 Hz, 1H), 7.06 (s, 1H), 7.13 (dd, J = 6.0 and 1.2 Hz, 1H), 7.56 (d, J = 7.2 Hz, 1H), 8.17 (d, J = 6.0 Hz, 1H); ESI-MS m/z 394.2 (MH⁺). IC50 (uM): 0.12 |
| 411 | 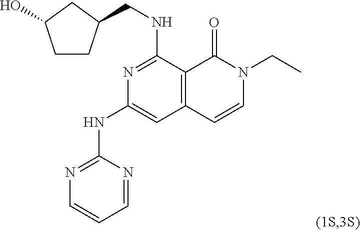 | ESI-MS m/z 381.2 (MH⁺). IC50 (uM): 0.071 |
| 412 | 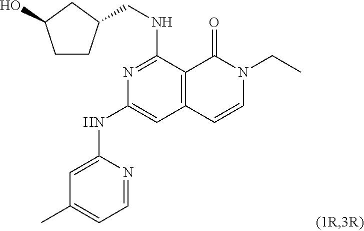 | ESI-MS m/z 394.2 (MH⁺). IC50 (uM): 0.224 |
| 413 | 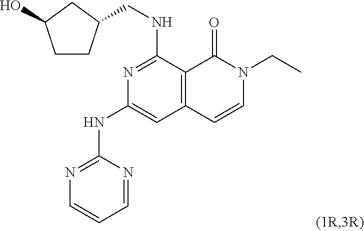 | ESI-MS m/z 381.2 (MH⁺). IC50 (uM): 0.0462 |
| 414 | 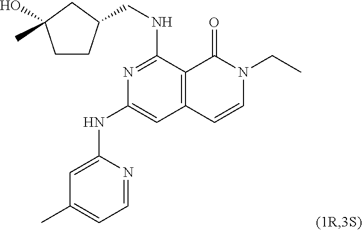 | ¹H NMR (MeOD-d₄) δ 1.34 (t, J = 6.8 Hz, 3H), 1.37 (s, 3H), 1.63~1.87 (m, 4H), 1.95~2.08 (m, 2H), 2.50 (s, 3H), 2.56 (m, 1H), 3.64 (d, J = 7.6 Hz, 2H), 4.01 (q, J = 7.2 Hz, 2H), 6.15 (s, 1H), 6.43 (d, J = 7.2 Hz, 1H), 7.04 (s, 1H), 7.12 (dd, J = 6.4 and 1.6 Hz, 1H), 7.55 (d, J = 7.2 Hz, 1H), 8.19 (d, J = 6.0 Hz, 1H); ESI-MS m/z 408.2 (MH⁺). IC50 (uM): 0.287 |
| 415 | 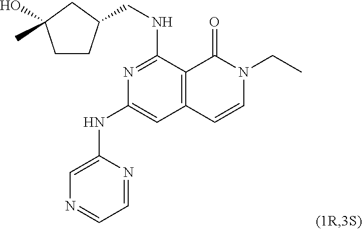 | ESI-MS m/z 395.2 (MH⁺). IC50 (uM): 0.172 |
| 416 | 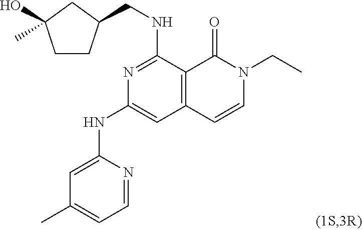 | ESI-MS m/z 408.2 (MH⁺). IC50 (uM): 0.176 |
| 417 | 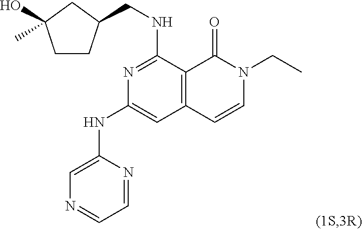 | ESI-MS m/z 395.2 (MH⁺). IC50 (uM): 0.0956 |
| 418 | 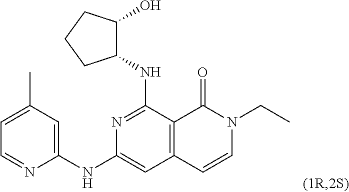 | ESI-MS m/z 380.2 (MH⁺). IC50 (uM): 0.182 |
| 419 | 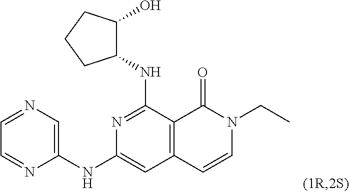 | ESI-MS m/z 367.2 (MH⁺). IC50 (uM): 0.0944 |
| 420 | 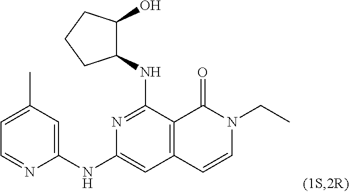 | ESI-MS m/z 380.2 (MH⁺). IC50 (uM): 0.011 |
| 421 | 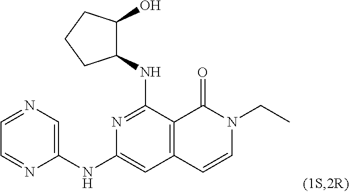 | ESI-MS m/z 367.2 (MH⁺). IC50 (uM): 0.0236 |
| 422 | 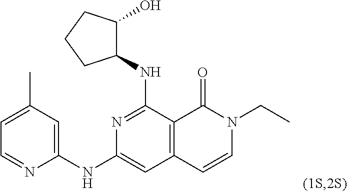 | ESI-MS m/z 380.2 (MH⁺). IC50 (uM): 0.0861 |
| 423 | 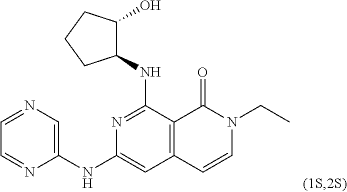 | ¹H NMR (MeOD-d₆) δ 1.25 (t, J = 7.2 Hz, 3H), 1.68~2.05 (m, 5H), 233 (m, 1H), 3.94 (q, J = 7.2 Hz, 2H), 3.98 (t, J = 6.8 Hz, 1H), 4.17 (q, J = 5.6 Hz, 1H), 6.21 (s, 1H), 6.42 (d, J = 7.2 Hz, 1H), 7.60 (d, J = 7.6 Hz, 1H), 8.23 (d, J = 2.8 Hz, 1H), 8.29 (dd, J = 2.8 and 1.6 Hz, 1H), 8.38 (d, J = 1.2 Hz, 1H); ESI-MS m/z 367.2 (MH⁺). IC50 (uM): 0.0524 |
| 424 | 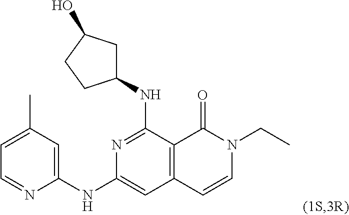 | ESI-MS m/z 380.2 (MH⁺). IC50 (uM): 0.047 |
| 425 | 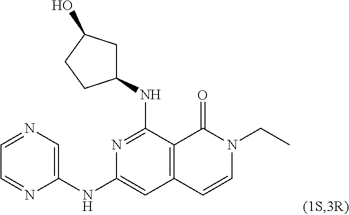 | ESI-MS m/z 367.2 (MH⁺). IC50 (uM): 0.316 |
| 426 | 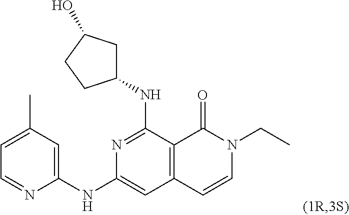 | ESI-MS m/z 380.2 (MH⁺). IC50 (uM): 0.0708 |
| 427 | 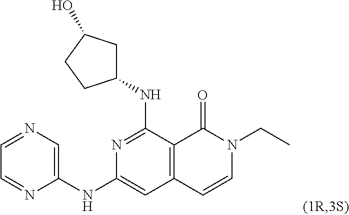 | ESI-MS m/z 367.2 (MH⁺). IC50 (uM): 0.0486 |
| 428 | 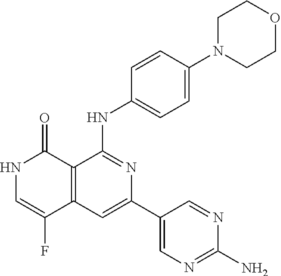 | ESI-MS m/z 434.17 (MH⁺). IC50 (uM): 0.108 |
| 429 | 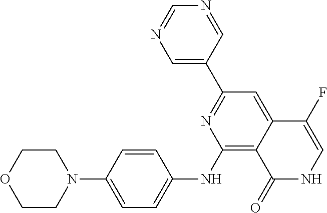 | ESI-MS m/z 419.16 (MH⁺). IC50 (uM): 0.055 |
| 430 | 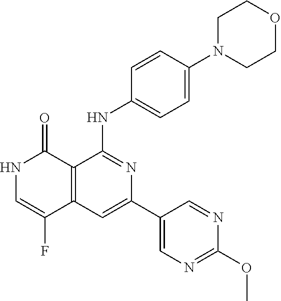 | ESI-MS m/z 449.17 (MH⁺). IC50 (uM): 0.12 |
| 431 | 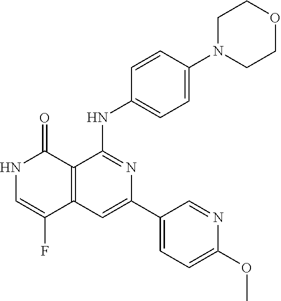 | ESI-MS m/z 432.18 (MH⁺). IC50 (uM): 0.287 |
| 432 | 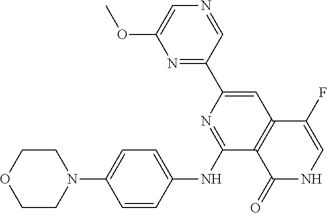 | ESI-MS m/z 449.17 (MH⁺). IC50 (uM): 0.623 |
| 433 | 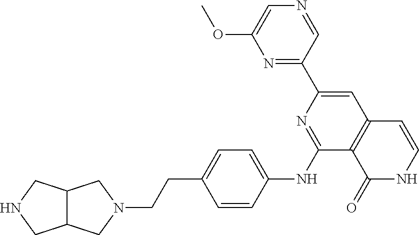 | ESI-MS m/z 484.24 (MH⁺). IC50 (uM): 0.051 |
| 434 | 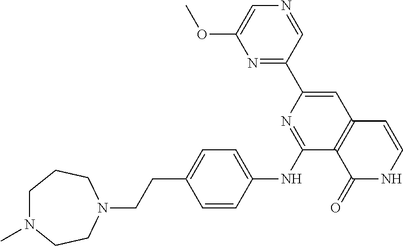 | ESI-MS m/z 486.25 (MH⁺). IC50 (uM): 0.037 |
| 435 | 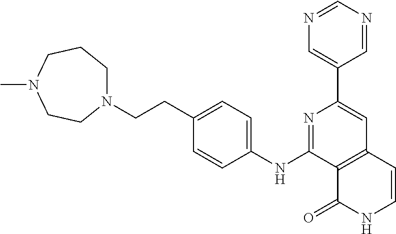 | ESI-MS m/z 456.24 (MH⁺). IC50 (uM): 0.086 |
| 436 | 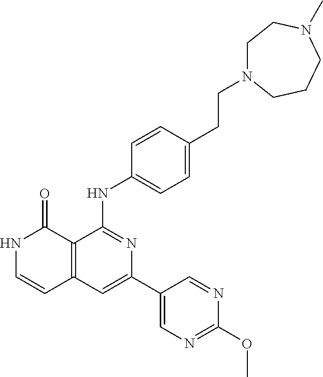 | ESI-MS m/z 486.25 (MH⁺). IC50 (uM): 0.14 |
| 437 | 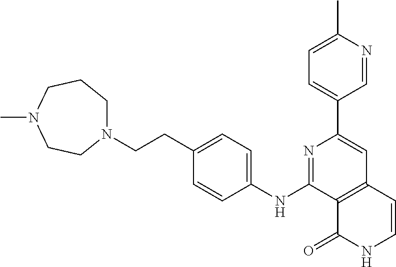 | ESI-MS m/z 469.26 (MH⁺). IC50 (uM): 0.102 |
| 438 | 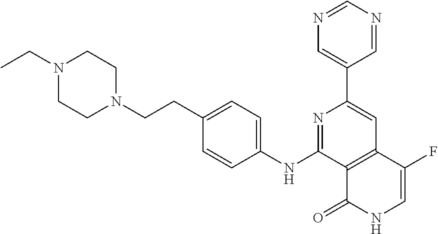 | ESI-MS m/z 474.23 (MH⁺). IC50 (uM): 0.289 |
| 439 | 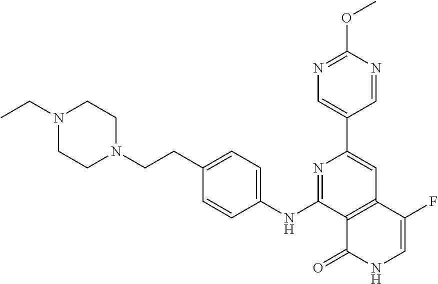 | ESI-MS m/z 504.24 (MH⁺). IC50 (uM): 0.474 |
| 440 | 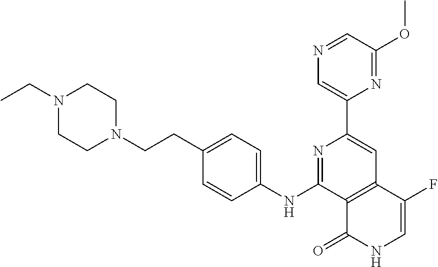 | ESI-MS m/z 504.24 (MH⁺). IC50 (uM): 0.343 |
| 441 | 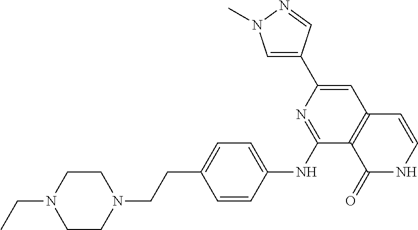 | ESI-MS m/z 458.26 (MH⁺). IC50 (uM): 0.058 |
| 442 | 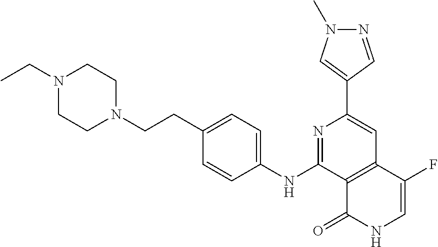 | ESI-MS m/z 476.25 (MH⁺). IC50 (uM): 0.107 |
| 443 | 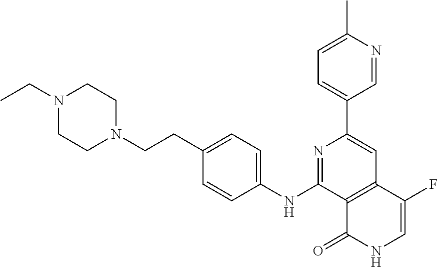 | ESI-MS m/z 487.25 (MH⁺). IC50 (uM): 0.419 |
| 444 | 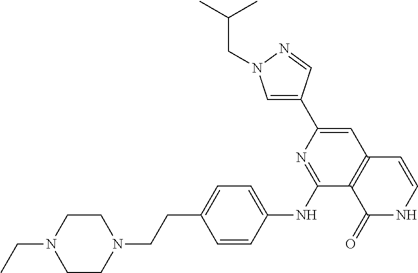 | ESI-MS m/z 500.31 (MH⁺). IC50 (uM): 0.122 |
| 445 | 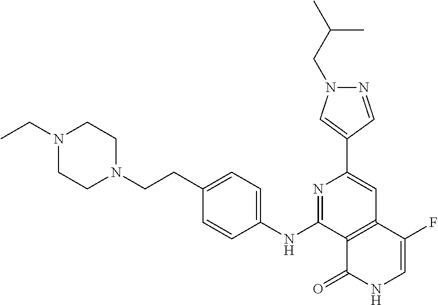 | ESI-MS m/z 518.30 (MH⁺). IC50 (uM): 0.30 |
| 446 | 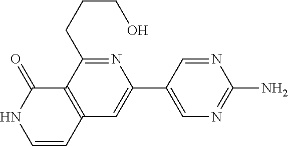 | ESI-MS m/z 299.12 (MH⁺). IC50 (uM): 0.086 |
| 447 | 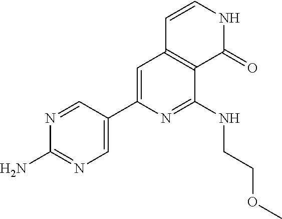 | ESI-MS m/z 313.13 (MH⁺). IC50 (uM): 0.101 |
| 448 | 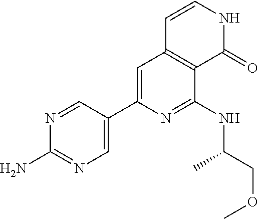 | ESI-MS m/z 327.15 (MH⁺). IC50 (uM): 0.125 |
| 449 | 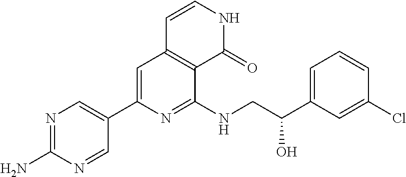 | ESI-MS m/z 409.11 (MH⁺). IC50 (uM): 0.384 |
| 450 | 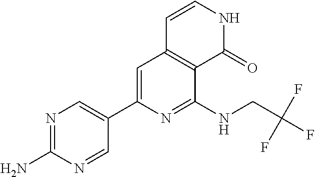 | ESI-MS m/z 337.09 (MH⁺). IC50 (uM): 0.117 |
| 451 | 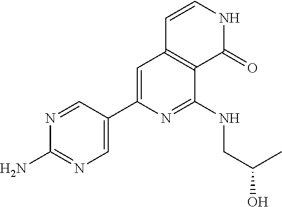 | ESI-MS m/z 313.13 (MH⁺). IC50 (uM): 0.088 |
| 452 | 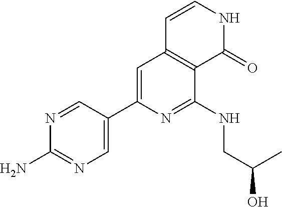 | ESI-MS m/z 313.13 (MH⁺). IC50 (uM): 0.14 |
| 453 | 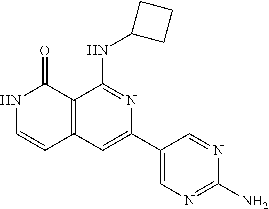 | ESI-MS m/z 309.14 (MH⁺). IC50 (uM): 0.162 |
| 454 | 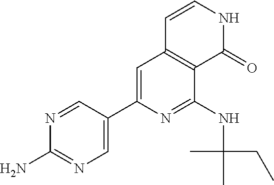 | ESI-MS m/z 325.17 (MH⁺). IC50 (uM): 0.281 |
| 455 | 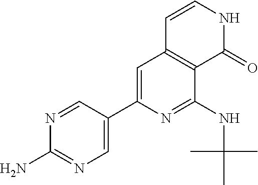 | ESI-MS m/z 311.15 (MH⁺). IC50 (uM): 0.059 |
| 456 | 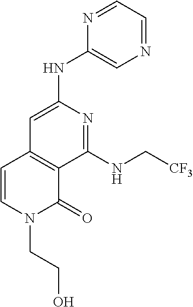 | ESI-MS m/z 381.12 (MH⁺). IC50 (uM): 0.0215 |
| 457 | 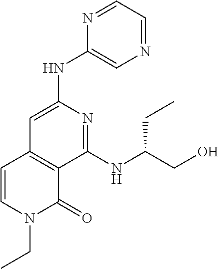 | ESI-MS m/z 355.18 (MH⁺). IC50 (uM): 0.302 |
| 458 | 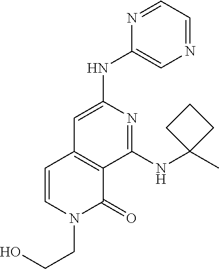 | ESI-MS m/z 367.18 (MH⁺). IC50 (uM): 0.0114 |
| 459 | 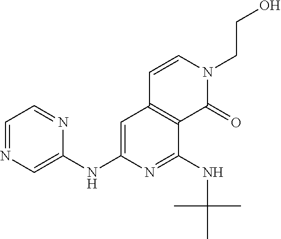 | ¹H NMR (400 MHz, MeOD): δ 8.51 (s, 1H), 8.37 (d, J = 3.2 Hz, 1H), 8.34 (d, J = 3.2 Hz, 1H), 7.68 (d, J = 7.2 Hz, 1H), 6.52 (d, J = 7.2 Hz, 1H), 6.32 (s, 1H), 4.11 (t, J = 5.2 Hz, 2H), 3.84 (t, J = 5.2 Hz, 2H), 1.71 (s, 9H); ESI-MS m/z 355.18 (MH+). IC50 (uM): 0.052 |
| 460 | 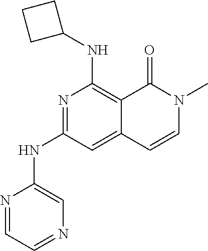 | ¹H NMR (400 MHz, MeOD): δ 8.57 (s, 1H), 8.34 (d, J = 2.4 Hz, 1H), 8.30 (d, J = 2.4 Hz, 1H), 7.60 (d, J = 7.2 Hz, 1H), 6.43 (d, J = 7.2 Hz, 1H), 6.28 (s, 1H), 4.40 (m, 1H), 3.52 (s, 3H), 2.67 (m, 1H), 2.23 (m, 1H), 2.07 (m, 1H); ESI-MS m/z 323.15 (MH+). IC50 (uM): 0.016 |
| 461 | 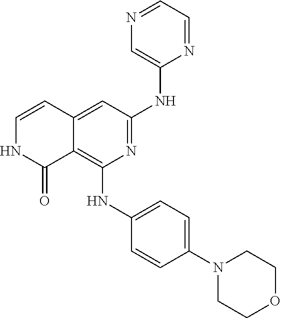 | ESI-MS m/z 416.18 (MH+). IC50 (uM): 0.10 |
| 462 | 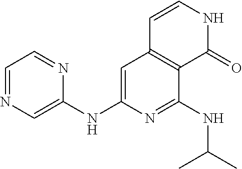 | ESI-MS m/z 297.14 (MH+). IC50 (uM): 0.03 |
| 463 | 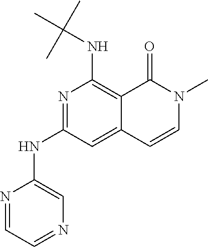 | ESI-MS m/z 325.17 (MH+). IC50 (uM): 0.054 |
| 464 | 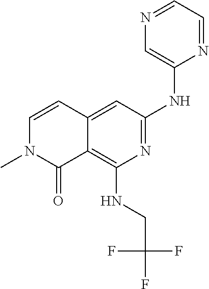 | ESI-MS m/z 351.11 (MH+). IC50 (uM): 0.081 |
| 465 | 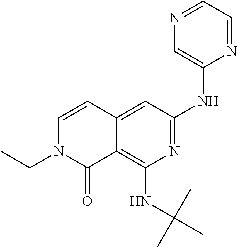 | ESI-MS m/z 339.19 (MH+). IC50 (uM): 0.147 |
| 466 | 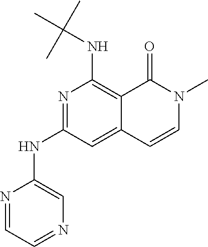 | ESI-MS m/z 325.17 (MH+). IC50 (uM): 0.12 |
| 467 | 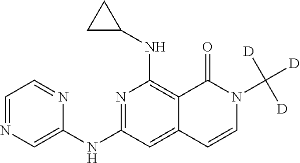 | ESI-MS m/z 312.16 (MH+). IC50 (uM): 0.049 |
| 468 | 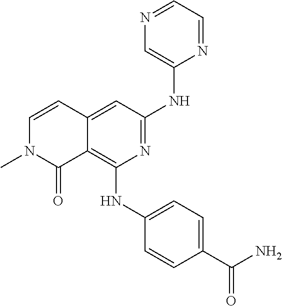 | ESI-MS m/z 388.14 (MH+). IC50 (uM): 0.101 |
| 469 | 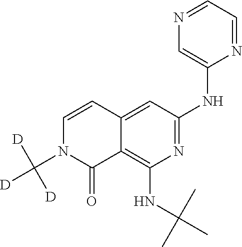 | ESI-MS m/z 328.19 (MH+). IC50 (uM): 0.097 |
| 470 | 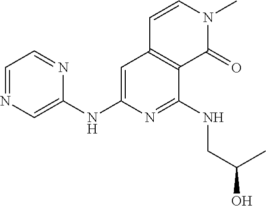 | ¹H NMR (400 MHz, MeOD): δ 8.48 (s, 1H), 8.33 (d, J = 2.1 Hz, 1H), 8.31 (d, J = 2.1 Hz, 1H), 7.64 (d, J = 7.6 Hz, 2H), 6.46 (d, J = 7.6 Hz, 1H), 6.30 (s, 1H), 4.18 (m, 1H), 3.73 (dd, J′ = 3.6 Hz, J″ = 3.6 Hz, 1H), 3.57 (dd, J′ = 3.6 Hz, J″ = 3.6 Hz, 1H), 3.56 (s, 3H), 1.37 (d, J = 6.4 Hz, 3H); ESI-MS m/z 327.15 (MH+). IC50 (uM): 0.082 |
| 471 | 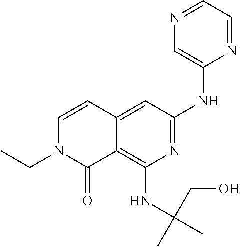 | ESI-MS m/z 355.18 (MH+). IC50 (uM): 0.10 |
| 472 | 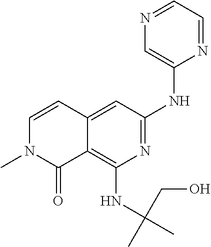 | ESI-MS m/z 341.16 (MH+). IC50 (uM): 0.155 |
| 473 | 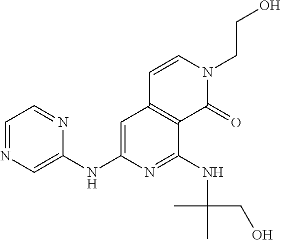 | ESI-MS m/z 371.17 (MH+). IC50 (uM): 0.114 |
| 474 | 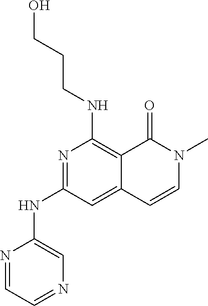 | ESI-MS m/z 327.15 (MH+). IC50 (uM): 0.14 |
| 475 | 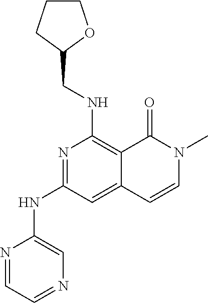 | ¹H NMR (400 MHz, MeOD): δ 8.50 (s, 1H), 8.32 (d, J = 1.2 Hz, 1H), 8.30 (d, J = 1.2 Hz, 1H), 7.63 (d, J = 7.6 Hz, 2H), 6.45 (d, J = 7.6 Hz, 1H), 6.30 (s, 1H), 4.27 (m, 1H), 3.84 (m, 1H), 3.78 (m, 1H), 3.68 (m, 1H), 3.65 (m, 1H), 3.54 (s, 3H), 2.17 (m. 1H), 2.01 (m, 2H), 1.83 (m, 1H); ESI-MS m/z 353.16 (MH+). IC50 (uM): 0.139 |
| 476 | 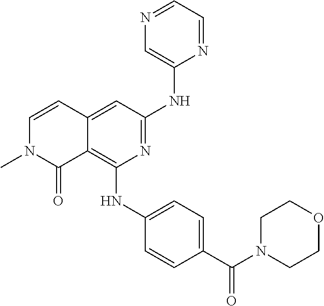 | ¹H NMR (400 MHz, MeOD): δ 8.50 (s, 1H), 8.17 (s, 1H), 7.87 (s, 1H), 7.78 (d, J = 8.4 Hz, 2H), 7.66 (d, J = 8.4 Hz, 2H), 7.63 (d, J = 7.2 Hz, 1H), 6.68 (s, 1H), 6.53 (d, J = 7.2 Hz, 1H), 3.74 (br, 8H), 3.59 (s, 3H); ESI-MS m/z 458.18 (MH+). IC50 (uM): 0.35 |
| 477 | 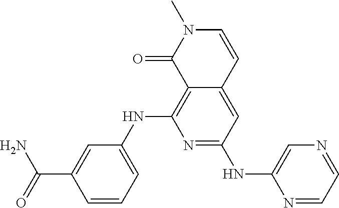 | ¹H NMR (400 MHz, MeOD); δ 8.32 (s, 1H), 8.25 (s, 1H), 8.04 (s, 1H), 7.79 (d, J = 7.2 Hz, 2H), 7.75 (d, J = 8.0 Hz, 2H), 7.69 (s, 1H), 7.58 (t, J = 8.0 Hz, 1H), 7.50 t, J = 7.6 Hz, 1H), 6.75 (s, 1H), 6.40 (d, J = 7.6 Hz, 1H), 3.53 (s, 3H); ESI-MS m/z 388.14 (MH+). IC50 (uM): 0.58 |
| 478 | 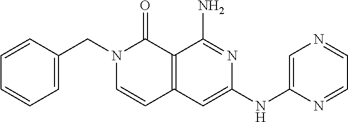 | ESI-MS m/z 345.14 (MH+). IC50 (uM): 1.633 |
| 479 | 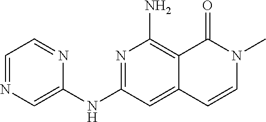 | ESI-MS m/z 269.11 (MH+). IC50 (uM): 0.599 |
| 480 | 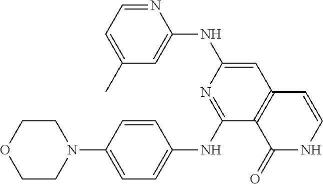 | ESI-MS m/z 429.20 (MH+). IC50 (uM): 0.015 |
| 481 | 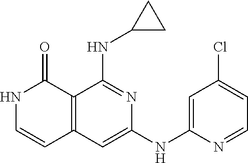 | ESI-MS m/z 328.09 (MH+). IC50 (uM): 0.122 |
| 482 | 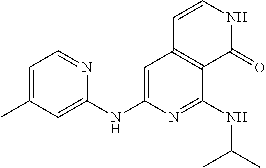 | ESI-MS m/z 309.16 (MH+). IC50 (uM): 0.053 |
| 483 | 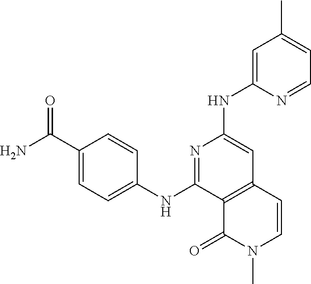 | ESI-MS m/z 401.16 (MH+). IC50 (uM): 0.062 |
| 484 | 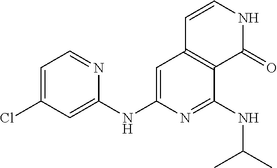 | ESI-MS m/z 330.10 (MH+). IC50 (uM): 0.054 |
| 485 | 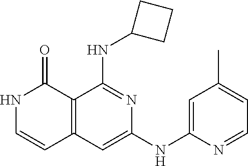 | ESI-MS m/z 322.16 (MH+). IC50 (uM): 0.045 |
| 486 | 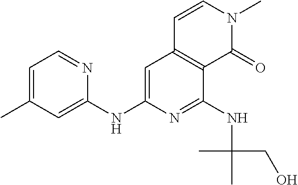 | ESI-MS m/z 354.19 (MH+). IC50 (uM): 0.045 |
| 487 | 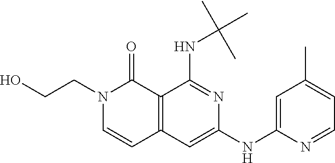 | ESI-MS m/z 368.20 (MH+). IC50 (uM): 0.102 |
| 488 | 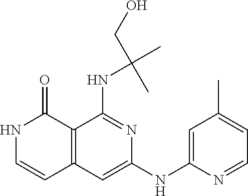 | ESI-MS m/z 340.17 (MH+). IC50 (uM): 0.021 |
| 489 | 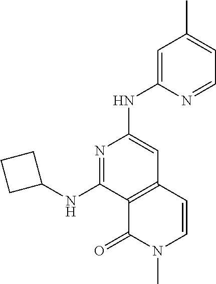 | ESI-MS m/z 336.17 (MH+). IC50 (uM): 0.124 |
| 490 | 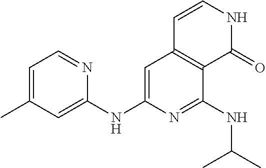 | ESI-MS m/z 310.16 (MH+). IC50 (uM): 0.032 |
| 491 | 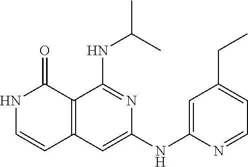 | ESI-MS m/z 324.17 (MH+). IC50 (uM): 0.063 |
| 492 | 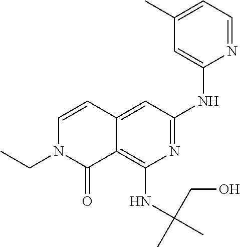 | ESI-MS m/z 368.20 (MH+). IC50 (uM): 0.054 |
| 493 | 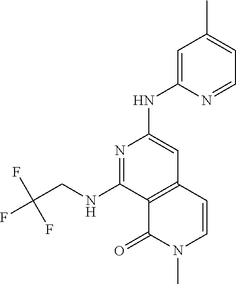 | ESI-MS m/z 364.13 (MH+). IC50 (uM): 0.214 |
| 494 | 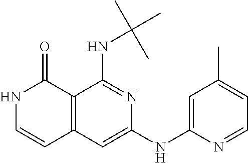 | ESI-MS m/z 324.17 (MH+). IC50 (uM): 0.075 |
| 495 | 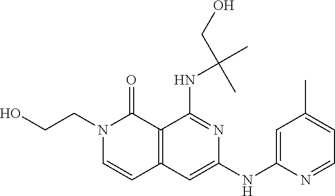 | ESI-MS m/z 384.20 (MH+). IC50 (uM): 0.052 |
| 496 | 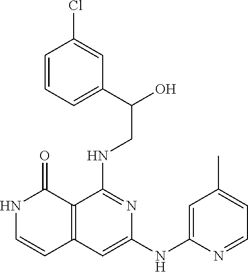 | ESI-MS m/z 422.13 (MH+). IC50 (uM): 0.115 |
| 497 | 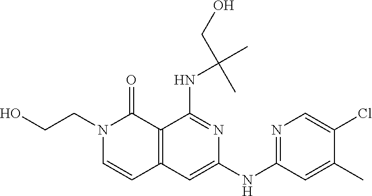 | ESI-MS m/z 418.16 (MH+). IC50 (uM): 0.111 |
| 498 | 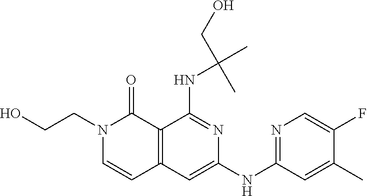 | ESI-MS m/z 402.19 (MH+). IC50 (uM): 0.076 |
| 499 | 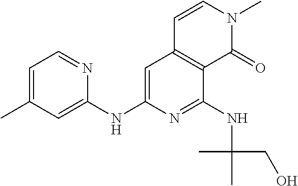 | ESI-MS m/z 354.19 (MH+). IC50 (uM): 0.031 |
| 500 | 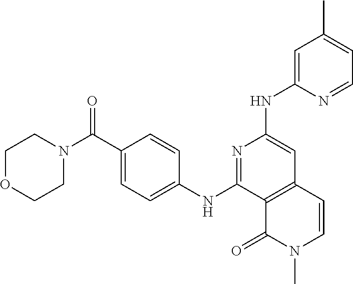 | ESI-MS m/z 471.21 (MH+). IC50 (uM): 0.069 |
| 501 | 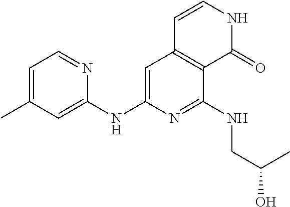 | ESI-MS m/z 326.15 (MH+). IC50 (uM): 0.038 |
| 502 | 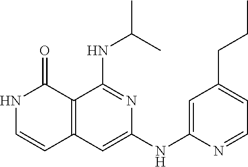 | ESI-MS m/z 338.19 (MH+). IC50 (uM): 0.164 |
| 503 | 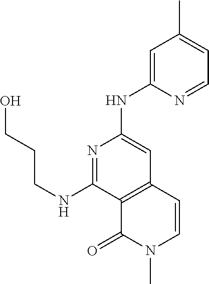 | ESI-MS m/z 340.17 (MH+). IC50 (uM): 0.084 |
| 504 | 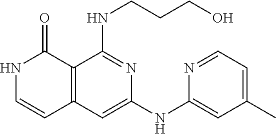 | ESI-MS m/z 326.15 (MH+). IC50 (uM): 0.048 |
| 505 | 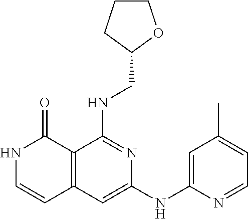 | ESI-MS m/z 352.17 (MH+). IC50 (uM): 0.063 |
| 506 | 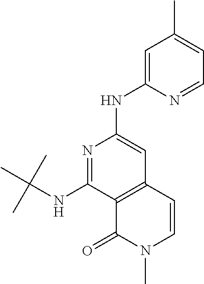 | ESI-MS m/z 338.19 (MH+). IC50 (uM): 0.129 |
| 507 | 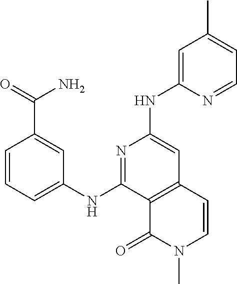 | ESI-MS m/z 401.16 (MH+). IC50 (uM): 0.136 |
| 508 | 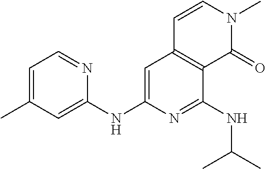 | ESI-MS m/z 324.17 (MH+). IC50 (uM): 0.153 |
| 509 | 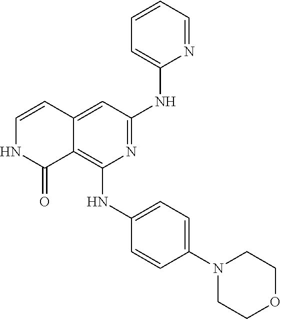 | ESI-MS m/z 415.18 (MH+). IC50 (uM): 0.143 |
| 510 | 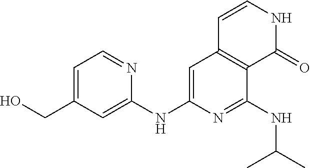 | ESI-MS m/z 326.15 (MH+). IC50 (uM): 0.034 |
| 511 | 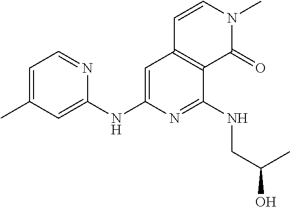 | ESI-MS m/z 340.17 (MH+). IC50 (uM): 0.086 |
| 512 | 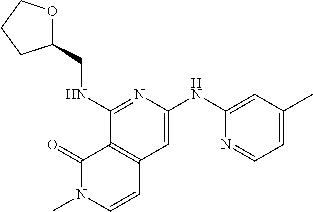 | ESI-MS m/z 366.18 (MH+). IC50 (uM): 0.158 |
| 513 | 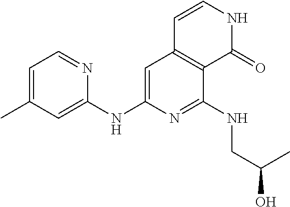 | ESI-MS m/z 326.15 (MH+). IC50 (uM): 0.07 |
| 514 | 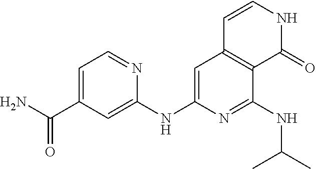 | ESI-MS m/z 339.15 (MH+). IC50 (uM): 0.051 |
| 515 | 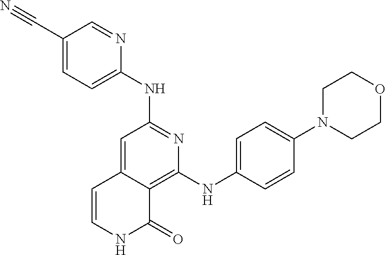 | ESI-MS m/z 440.17 (MH+). IC50 (uM): 0.301 |
| 516 | 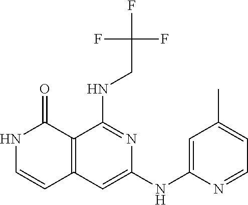 | ESI-MS m/z 350.12 (MH+). IC50 (uM): 0.152 |
| 517 | 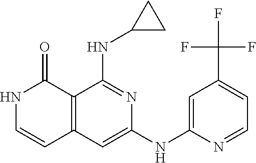 | ESI-MS m/z 362.12 (MH+). IC50 (uM): 0.386 |
| 518 | 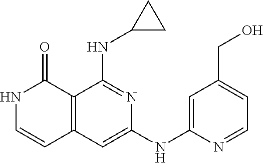 | ESI-MS m/z 324.14 (MH+). IC50 (uM): 0.059 |
| 519 | 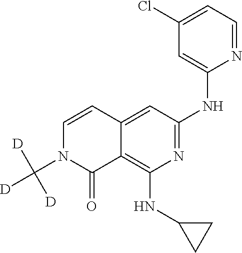 | ESI-MS m/z 345.12 (MH+). IC50 (uM): 0.302 |
| 520 | 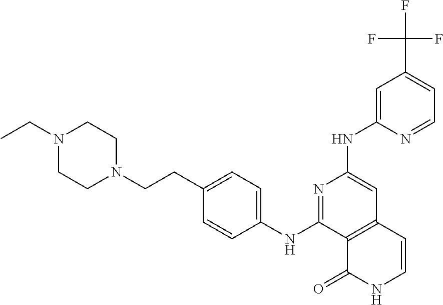 | ESI-MS m/z 538.25 (MH+). IC50 (uM): 0.183 |
| 521 | 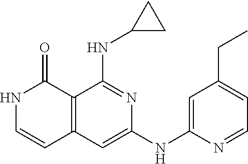 | ESI-MS m/z 322.16 (MH+). IC50 (uM): 0.131 |
| 522 | 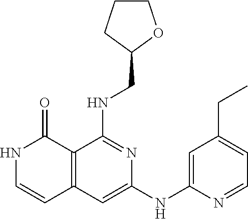 | ESI-MS m/z 352.17 (MH+). IC50 (uM): 0.083 |
| 523 | 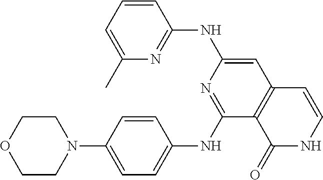 | ESI-MS m/z 428.20 (MH+). IC50 (uM): 0.447 |
| 524 | 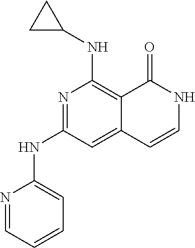 | ESI-MS m/z 294.13 (MH+). IC50 (uM): 0.199 |
| 525 | 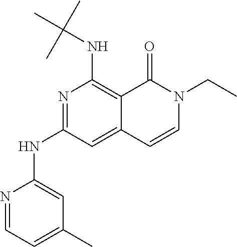 | ESI-MS m/z 352.21 (MH+). IC50 (uM): 0.242 |
| 526 | 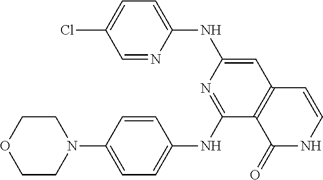 | ESI-MS m/z 449.14 (MH+). IC50 (uM): 0.803 |
| 527 | 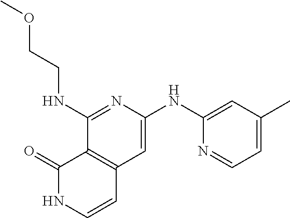 | ESI-MS m/z 326.15 (MH+). IC50 (uM): 0.12 |
| 528 | 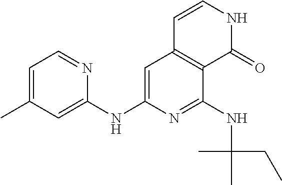 | ESI-MS m/z 338.19 (MH+). IC50 (uM): 0.374 |
| 529 | 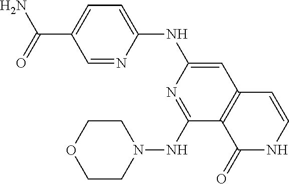 | ESI-MS m/z 458.119 (MH+). IC50 (uM): 0.85 |
| 530 | 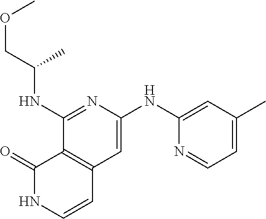 | ESI-MS m/z 340.17 (MH+). IC50 (uM): 0.097 |
| 531 | 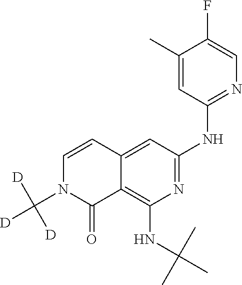 | ESI-MS m/z 359.20 (MH+). IC50 (uM): 0.296 |
| 532 | 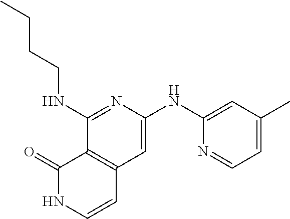 | ESI-MS m/z 324.17 (MH+). IC50 (uM): 0.25 |
| 533 | 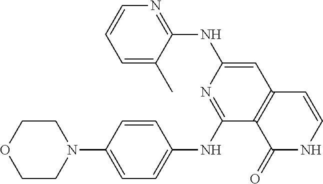 | ESI-MS m/z 429.20 (MH+). IC50 (uM): 0.382 |
| 534 | 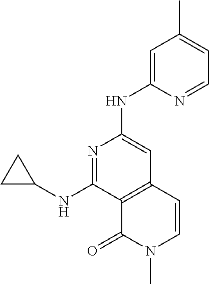 | ESI-MS m/z 322.16 (MH+). IC50 (uM): 0.129 |
| 535 | 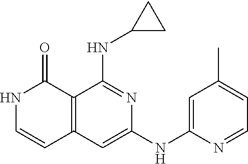 | ESI-MS m/z 308.14 (MH+). IC50 (uM): 0.206 |
| 536 | 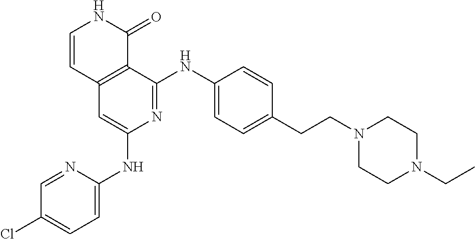 | ESI-MS m/z 504.22 (MH+). IC50 (uM): 0.398 |
| 537 | 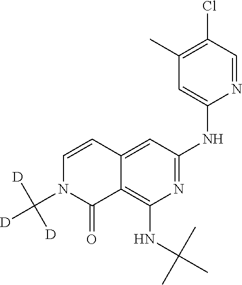 | ESI-MS m/z 375.17 (MH+). IC50 (uM): 0.502 |
| 538 | 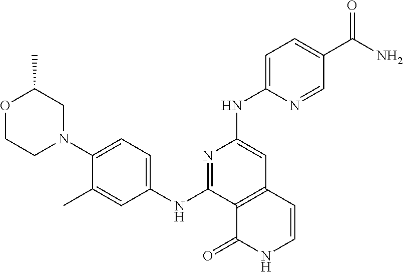 | ESI-MS m/z 486.22 (MH+). IC50 (uM): 0.361 |
| 539 | 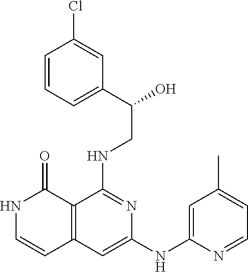 | ESI-MS m/z 422.13 (MH+). IC50 (uM): 0.915 |
| 540 | 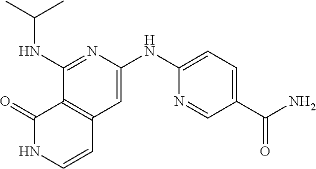 | ESI-MS m/z 339.15 (MH+). IC50 (uM): 0.206 |
| 541 | 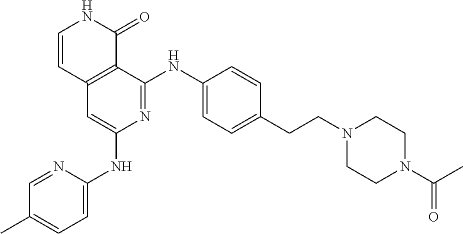 | ESI-MS m/z 498.25 (MH+). IC50 (uM): 1.124 |
| 542 | 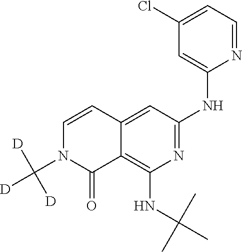 | ESI-MS m/z 361.15 (MH+). IC50 (uM): 0.83 |
| 543 | 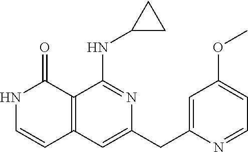 | ESI-MS m/z 324.14 (MH+). IC50 (uM): 0.266 |
| 544 | 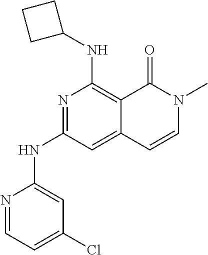 | ESI-MS m/z 356.12 (MH+). IC50 (uM): 0.81 |
| 545 | 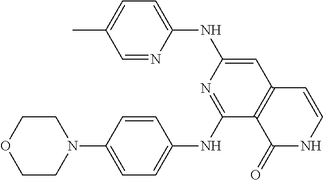 | ESI-MS m/z 429.20 (MH+). IC50 (uM): 3.85 |
| 546 | 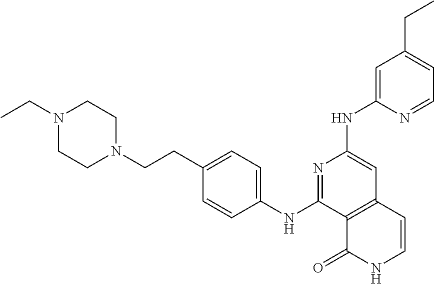 | ESI-MS m/z 498.29 (MH+). IC50 (uM): 0.088 |
| 547 | 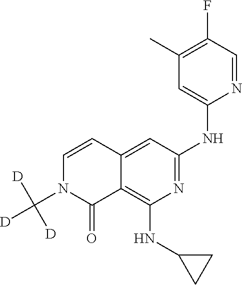 | ESI-MS m/z 343.17 (MH+). IC50 (uM): 0.415 |
| 548 | 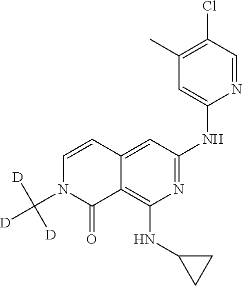 | ESI-MS m/z 359.14 (MH+). IC50 (uM): 0.468 |
| 549 | 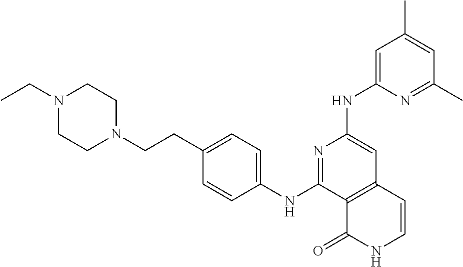 | ESI-MS m/z 498.29 (MH+). |
| 550 | 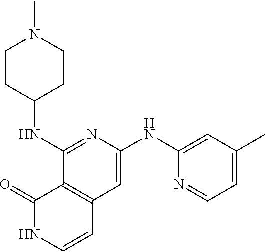 | ESI-MS m/z 365.20 (MH+). IC50 (uM): 0.111 |
| 551 | 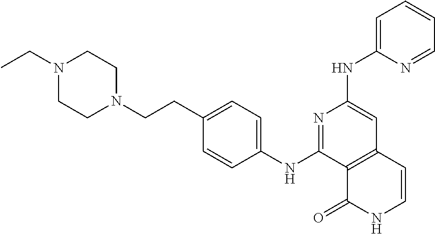 | ESI-MS m/z 470.26 (MH+). IC50 (uM): 0.20 |
| 552 | 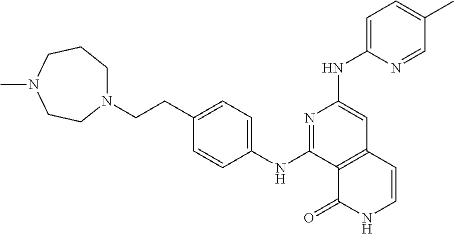 | ESI-MS m/z 484.27 (MH+). IC50 (uM): 0.39 |
| 553 | 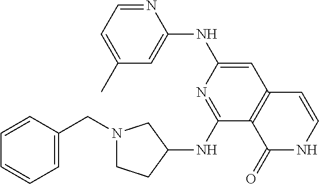 | ESI-MS m/z 427.22 (MH+). IC50 (uM): 0.55 |
| 554 | 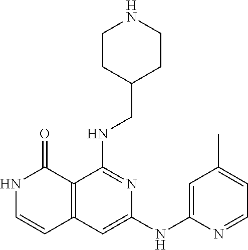 | ESI-MS m/z 365.20 (MH+). IC50 (uM): 0.086 |
| 555 | 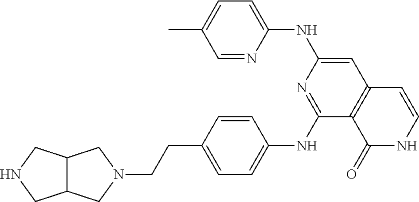 | ESI-MS m/z 422.26 (MH+). IC50 (uM): 0.393 |
| 556 | 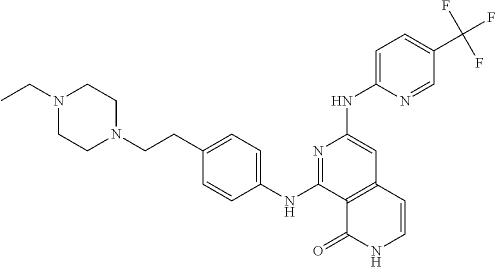 | ESI-MS m/z 538.25 (MH+). IC50 (uM): 0.631 |
| 557 | 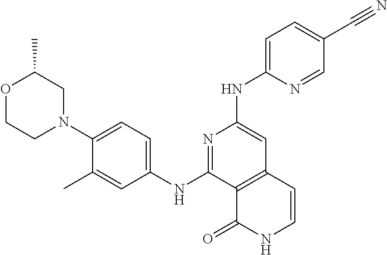 | ESI-MS m/z 468.21 (MH+). IC50 (uM): 3.35 |
| 558 | 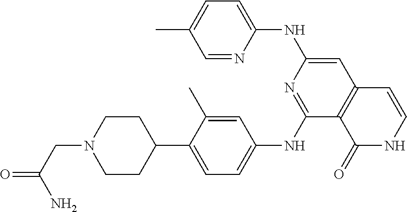 | ESI-MS m/z 498.25 (MH+). IC50 (uM): 0.489 |
| 559 | 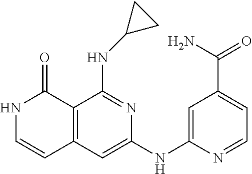 | ESI-MS m/z 337.13 (MH+). IC50 (uM): 0.292 |
| 560 | 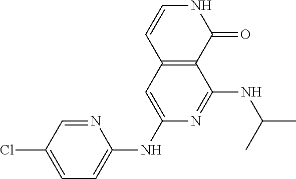 | ESI-MS m/z 330.10 (MH+). IC50 (uM): 0.611 |
| 561 | 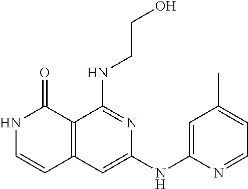 | ESI-MS m/z 312.14 (MH+). IC50 (uM): 0.501 |
| 562 | 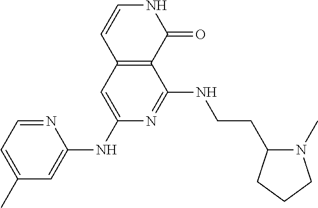 | ESI-MS m/z 379.22 (MH+). IC50 (uM): 0.288 |
| 563 | 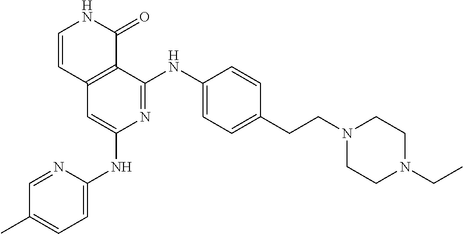 | ESI-MS m/z 484.27 (MH+). IC50 (uM): 0.43 |
| 564 | 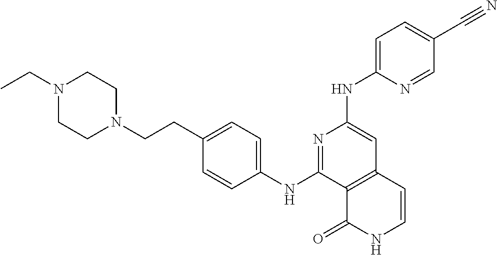 | ESI-MS m/z 495.25 (MH+). IC50 (uM): 0.385 |
| 565 | 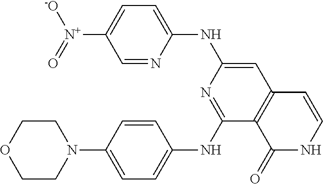 | ESI-MS m/z 460.17 (MH+). IC50 (uM): 0.71 |
| 566 | 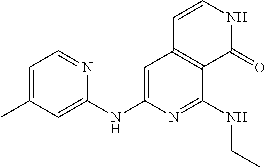 | ESI-MS m/z 296.14 (MH+). IC50 (uM): 0.803 |
| 567 | 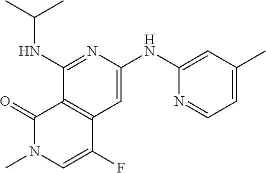 | ESI-MS m/z 342.17 (MH+). IC50 (uM): 0.978 |
| 568 | 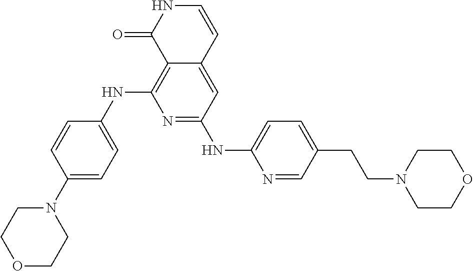 | ESI-MS m/z 528.26 (MH+). IC50 (uM): 0.549 |
| 569 | 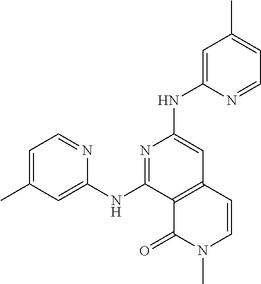 | ESI-MS m/z 373.17 (MH+). IC50 (uM): 0.701 |
| 570 | 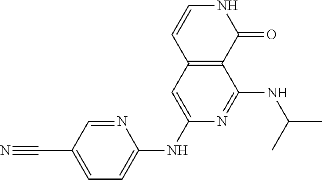 | ESI-MS m/z 321.14 (MH+). IC50 (uM): 0.377 |
| 571 | 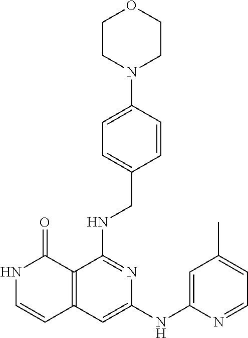 | ESI-MS m/z 343.21 (MH+). IC50 (uM): 2.96 |
| 572 | 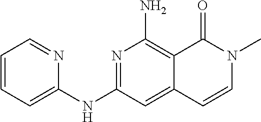 | ESI-MS m/z 268.11 (MH+). IC50 (uM): 3.268 |
| 573 | 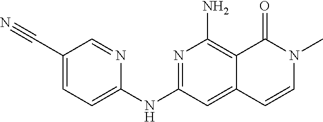 | ESI-MS m/z 293.11 (MH+). IC50 (uM): 3.867 |
| 574 | 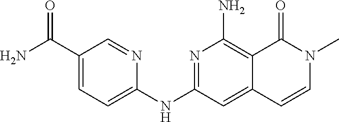 | ESI-MS m/z 311.12 (MH+). IC50 (uM): 0.142 |
| 575 | 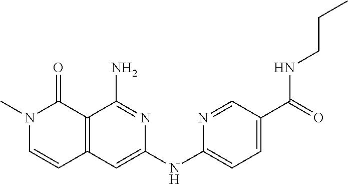 | ESI-MS m/z 353.16 (MH+). IC50 (uM): 2.61 |
| 576 | 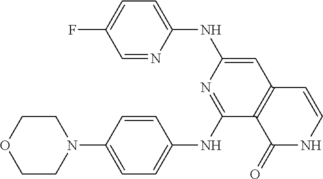 | ESI-MS m/z 433.17 (MH+). IC50 (uM): 4.87 |
| 577 | 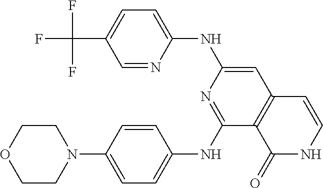 | ESI-MS m/z 483.17 (MH+). IC50 (uM): 0.76 |
| 578 | 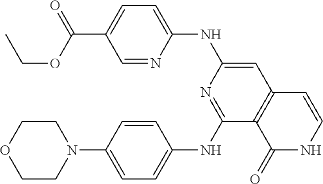 | ESI-MS m/z 487.20 (MH+). IC50 (uM): 0.186 |
| 579 | 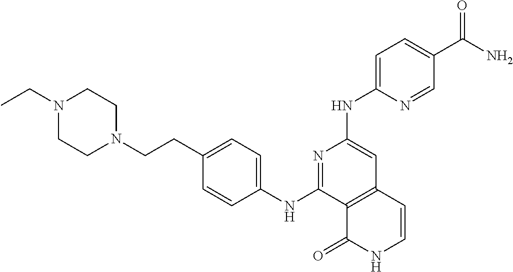 | ESI-MS m/z 513.26 (MH+). IC50 (uM): 0.113 |
| 580 | 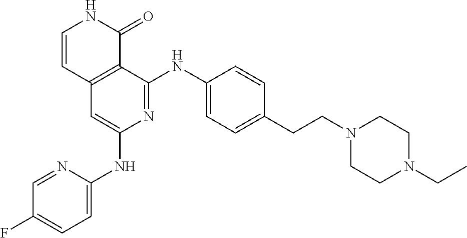 | ESI-MS m/z 488.25 (MH+). IC50 (uM): 0.163 |
| 581 | 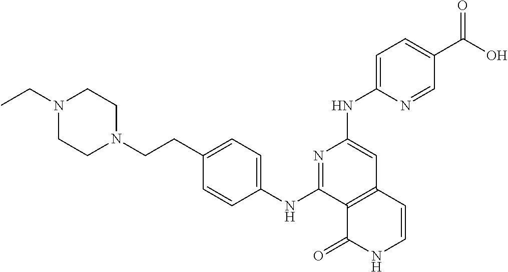 | ESI-MS m/z 514.25 (MH+). IC50 (uM): 0.016 |
| 582 | 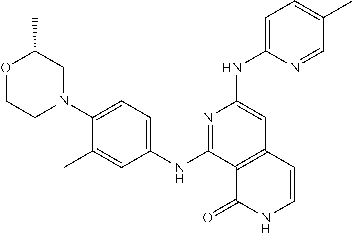 | ESI-MS m/z 457.23 (MH+). IC50 (uM): 2.22 |
| 583 | 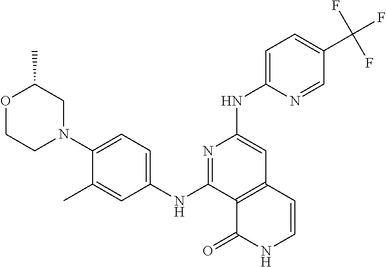 | ESI-MS m/z 511.20 (MH+). IC50 (uM): 8.44 |
| 584 | 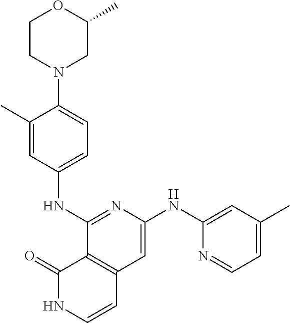 | ESI-MS m/z 457.23 (MH+). IC50 (uM): 2.68 |
| 585 | 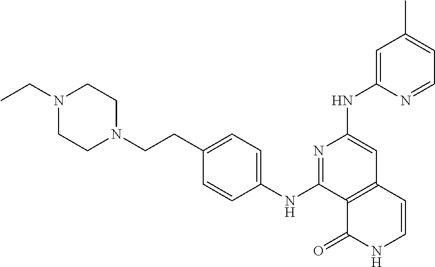 | ESI-MS m/z 484.27 (MH+). IC50 (uM): 0.081 |
| 586 | 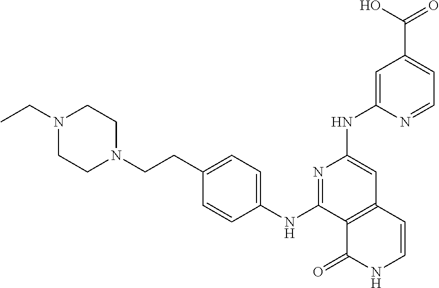 | ESI-MS m/z 514.25 (MH+). IC50 (uM): 0.226 |
| 587 | 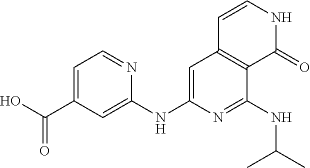 | ESI-MS m/z 340.13 (MH+). IC50 (uM): 0.226 |
| 588 | 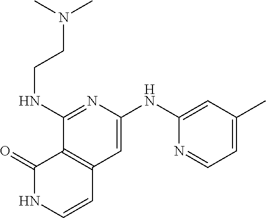 | ESI-MS m/z 339.19 (MH+). IC50 (uM): 1.96 |
| 589 | 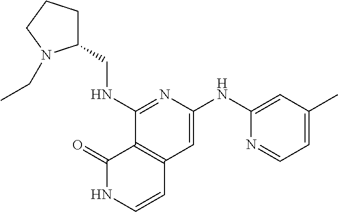 | ESI-MS m/z 379.22 (MH+). IC50 (uM): 5.56 |
| 590 | 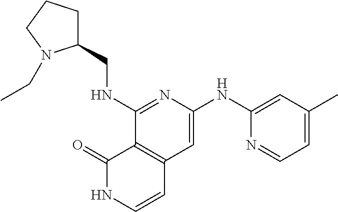 | ESI-MS m/z 379.22 (MH+). IC50 (uM): 3.50 |
| 591 | 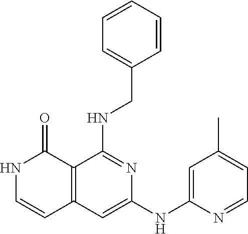 | ESI-MS m/z 358.16 (MH+). IC50 (uM): 5.68 |
| 592 | 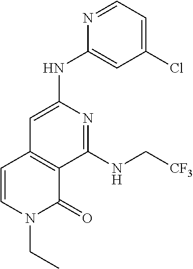 | ESI-MS m/z 398.09 (MH+). IC50 (uM): 0.962 |
| 593 | 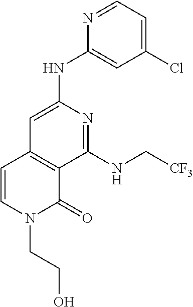 | ESI-MS m/z 414.09 (MH+). IC50 (uM): 0.271 |
| 594 | 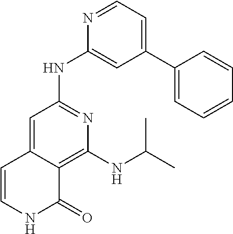 | ESI-MS m/z 327.17 (MH+). IC50 (uM): 2.225 |
| 595 | 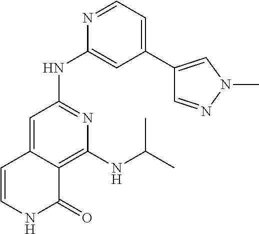 | ESI-MS m/z 376.18 (MH+). IC50 (uM): 0.0879 |
| 596 | 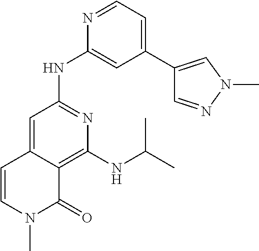 | ESI-MS m/z 392.20 (MH+). IC50 (uM): 0.141 |
| 597 | 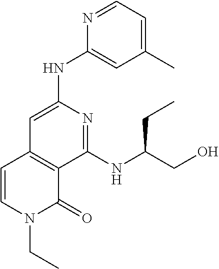 | ESI-MS m/z 368.20 (MH+). IC50 (uM): 0.081 |
| 598 | 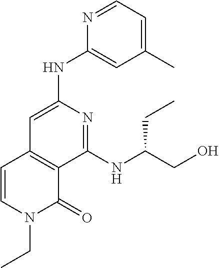 | ESI-MS m/z 368.20 (MH+). IC50 (uM): 0.518 |
| 599 | 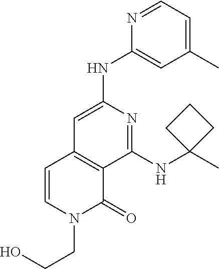 | ESI-MS m/z 380.20 (MH+). IC50 (uM): 0.286 |
| 600 | 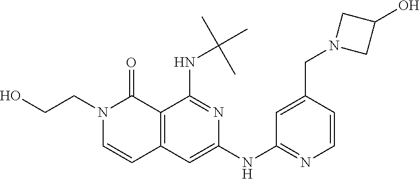 | ESI-MS m/z 439.2 (MH⁺). ¹H NMR (MeOD-d₄) [symbol] 8.13(d, J = 5.6 Hz, 1H), 7.76 (s, 1H), 7.13 (d, J = 7.2 Hz, 1H), 6.94 (d, J = 5.2 Hz, 1H), 6.59 (s, 1H), 6.15 (d, J = 7.2 Hz, 1H), 3.89 (m, 2H), 3.71 (m, 2H), 2.56 (s, 1H), 1.46 (s, 9H), IC50 (uM): 0.0186 |
| 601 | 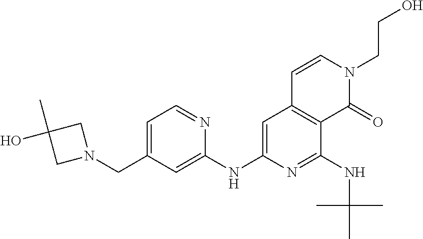 | ESI-MS m/z 452.6 (MH⁺). IC50 (uM): — |
| 602 | 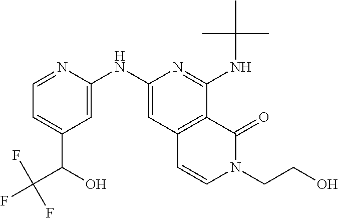 | ESI-MS m/z 452.2 (MH⁺). IC50 (uM): 0.065 |
| 603 | 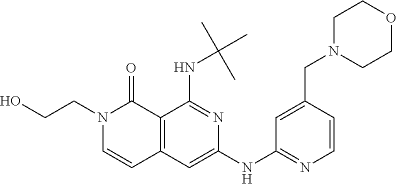 | ESI-MS m/z 453.6 (MH⁺). IC50 (uM): 0.056 |
| 604 | 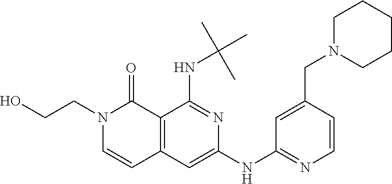 | ESI-MS m/z 451.3 (MH⁺). IC50 (uM): 0.251 |
| 605 | 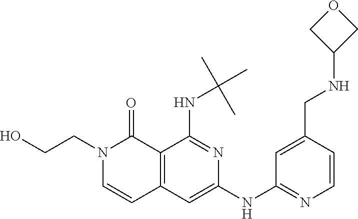 | ESI-MS m/z 439.3 (MH⁺). IC50 (uM): 0.216 |
| 606 | 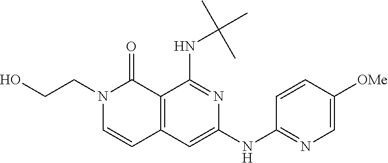 | ESI-MS m/z 384.3 (MH⁺). IC50 (uM): 0.793 |
| 607 | 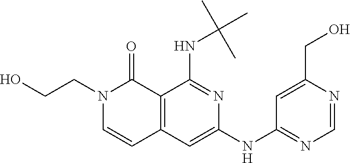 | ESI-MS m/z 385.2 (MH⁺). IC50 (uM): 0.029 |
| 608 | 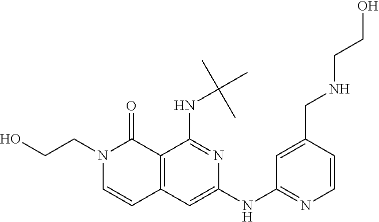 | ESI-MS m/z 427.3 (MH⁺). IC50 (uM): <0.00565 |
| 609 | 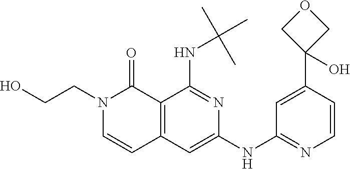 | ESI-MS m/z 426.2 (MH⁺). IC50 (uM): 0.012 |
| 610 | 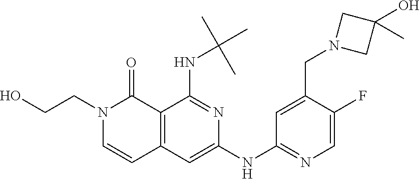 | ESI-MS m/z 471.2 (MH⁺). IC50 (uM): 0.37 |
| 611 | 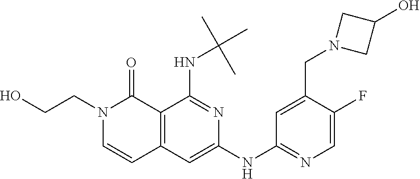 | ESI-MS m/z 457.2 (MH⁺). IC50 (uM): 0.251 |
| 612 | 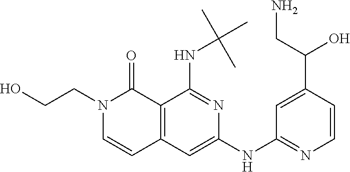 | ESI-MS m/z 413.2 (MH⁺). IC50 (uM): 0.453 |
| 613 | 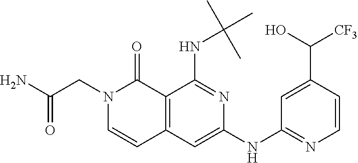 | ESI-MS m/z 465.2 (MH⁺). ¹H NMR (MeOD-d₄) [symbol] 9.47 (s, 1H), 8.13 (d, J = 5.2 Hz, 1H), 7.75 (s, 1H), 7.07 (d, J = 7.2 Hz, 1H), 6.95 (d, J = 5.2 Hz, 1H), 6.60 (s, 1H), 6.17 (d, J = 7.2 HZ, 1H), 4.95 (m, 1H), 4.44 (s, 1H), 1.45 (s, 9H). IC50 (uM): 0.142 |
| 614 | 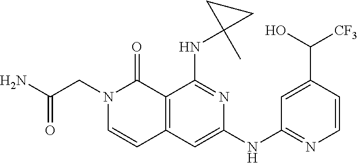 | ESI-MS m/z 463.2 (MH⁺). IC50 (uM): 0.066 |
| 615 | 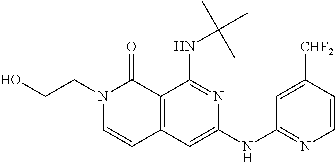 | ESI-MS m/z 404.2 (MH⁺). IC50 (uM): 0.009 |
| 616 | 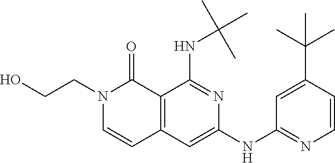 | ESI-MS m/z 41 IC50 (uM): 0.3 (MH⁺). IC50 (uM): 1.349 |
| 617 | 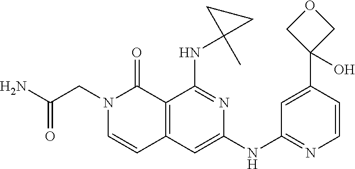 | ESI-MS m/z 437.2 (MH⁺). IC50 (uM): 0.064 |
| 618 | 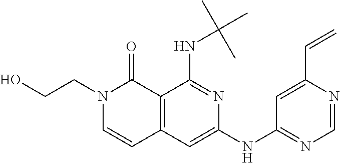 | ESI-MS m/z 381.2 (MH⁺). IC50 (uM): 0.121 |
| 619 | 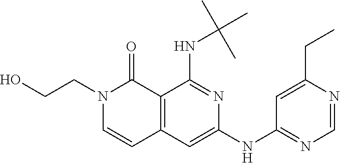 | ESI-MS m/z 383.2 (MH⁺). IC50 (uM): 0.102 |
| 620 | 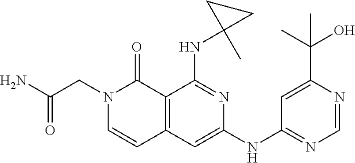 | ESI-MS m/z 424.2 (MH⁺). IC50 (uM): 0.148 |
| 621 | 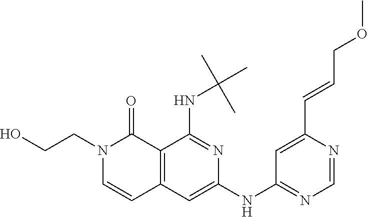 | ESI-MS m/z 425.2 (MH⁺). ¹H NMR (MeOD-d₄) [symbol] 8.50 (s, 1H),7.59 (s, 1H), 7.19 (d, J = 7.6 Hz, 1H), 6.83 (dt, J = 8.8 Hz, 5.6 Hz, 1H), 6.75 (s, 1H), 6.50 (dt, J = 14 Hz, 1H), 6.19 (d, J = 7.2 Hz, 1H), 4.07 (m, 2H), 3.91 (m, 2H), 3.72 (m, 2H), 3.32 (s, 3H), 1.48 (s, 9H). IC50 (uM): 0.094 |
| 622 | 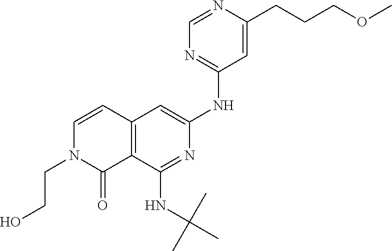 | ESI-MS m/z 427.2 (MH⁺). ¹H NMR (MeOD-d₄) [symbol] 8.49 (s, 1H), 7.63 (s, 1H), 7.18 (d, J = 7.2 Hz, 1H), 6.68 (s, 1H), 6.18 (d, J = 7.2 Hz, 1H), 3.91 (m, 2H), 3.71 (m, 2H), 3.33 (m, 2H), 3.23 (s, 3H), 2.63 (m, 2H), 1.86 (m, 2H), 1.46 (s, 9H). IC50 (uM): 0.085 |
| 623 | 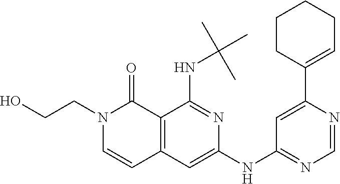 | ESI-MS m/z 435.2 (MH⁺). ¹H NMR (MeOD-d₄) [symbol] 8.50 (s, 1H), 7.41 (s, 1H), 7.19 (d, J = 7.2 Hz, 1H), 6.96 (s, 1H), 6.72 (m, 1H), 6.21 (d, J = 7.2 Hz, 1H), 5.40 (s, 1H), 3.91 (t, J = 5.6 Hz, 2H), 3.72 (t, J = 5.6 Hz, 2H), 2.34 (m, 2H), 2.19 (m, 2H), 1.72 (m, 2H), 1.63 (m, 2H), 1.47 (s, 9H). IC50 (uM): 0.612 |
| 624 | 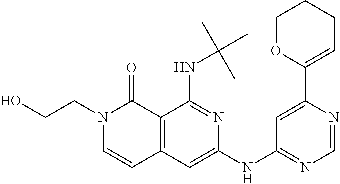 | ESI-MS m/z 437.2 (MH⁺). IC50 (uM): 0.59 |
| 625 | 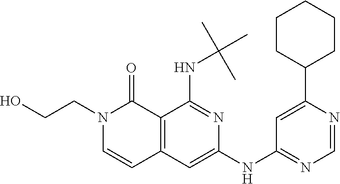 | ESI-MS m/z 437.2 (MH⁺). IC50 (uM): 0.452 |
| 626 | 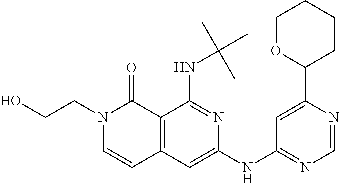 | ESI-MS m/z 439.2 (MH⁺). IC50 (uM): 0.756 |
| 627 | 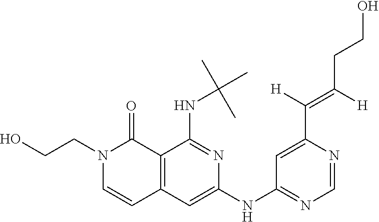 | ESI-MS m/z 425.2 (MH⁺). IC50 (uM): 0.06 |
| 628 | 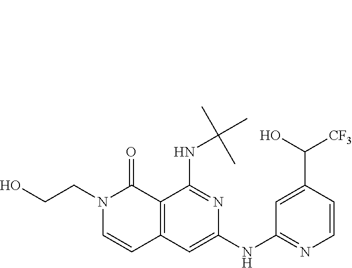 | ESI-MS m/z 466.2 (MH⁺). ¹H NMR (MeOD-d₄) [symbol] 9.60 (s, 1H), 8.23 (d, J = 5.2 Hz, 1H), 7.67 (s, 1H), 7.23 (d, J = 7.2 Hz, 1H), 7.10 (d, J = 5.2 Hz, 1H), 6.93 (s, 1H), 6.26 (d, J = 7.2 Hz, 1H), 4.00 (t, J = 5.2 Hz, 2H), 3.87 (t, J = 5.2 Hz, 2H0, 1.73 (s, 3H), 1.55 (s, 9H). IC50 (uM): 0.131 |
| 629 | 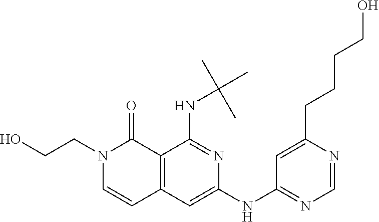 | ESI-MS m/z 427.2 (MH⁺). IC50 (uM): 0.062 |
| 630 | 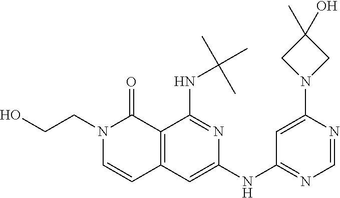 | ESI-MS m/z 440.2 (MH⁺). IC50 (uM): 0.204 |
| 631 | 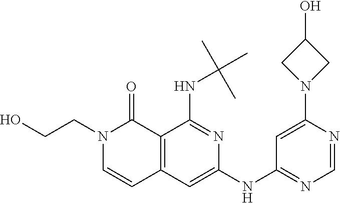 | ESI-MS m/z 426.2 (MH⁺). ¹H NMR (MeOD-d₄) [symbol] 9.80 (s, 1H), 7.16 (d, J = 7.2 Hz, 1H), 6.62 (m, 1H), 6.35 (s, 1H), 6.14 (d, J = 7.2 Hz, 1H), 4.61 (m, 1H), 4.21 (m, 2H), 3.89 (t, J = 5.2 Hz, 2H), 3.77 (m, 2H), 3.7l (t, J = 5.2 Hz, 2H), 1.48 (s, 9H). IC50 (uM): 0.233 |
| 632 | 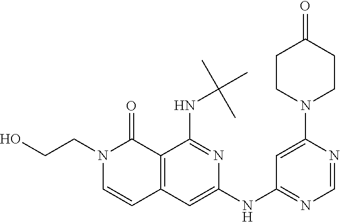 | ESI-MS m/z 452.2 (MH⁺). IC50 (uM): 0.117 |
| 633 | 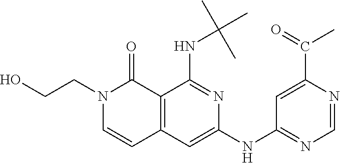 | ESI-MS m/z 397.2 (MH⁺). IC50 (uM): 0.273 |
| 634 | 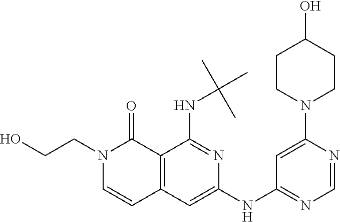 | ESI-MS m/z 454.3 (MH⁺). IC50 (uM): 0.161 |
| 635 | 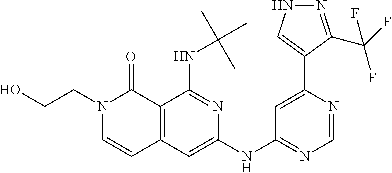 | ESI-MS m/z 489.2 (MH⁺). ¹H NMR (MeOD-d₄) [symbol] 9.51 (s, 1H), 8.58 (s, 1H), 8.09 (s, 1H), 7.46 (s, 1H), 7.18 (d, J = 7.2 Hz, 1H), 7.10 (s, 1H), 6.21 (d, J = 7.2 Hz, 1H), 3.90 (t, d = 5.2 Hz, 2H), 3.72 (t, J = 5.2 Hz, 2H), 1.42 (s, 9H). IC50 (uM): 0.314 |
| 636 | 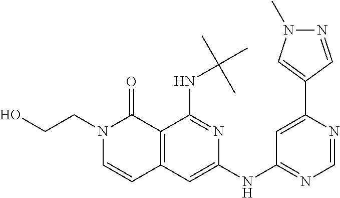 | ESI-MS m/z 435.2 (MH⁺). IC50 (uM): 0.237 |
| 637 | 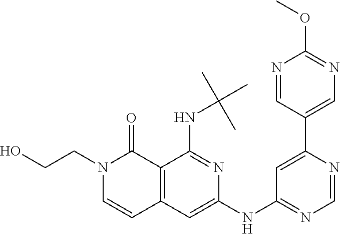 | ESI-MS m/z 463.2 (MH⁺). IC50 (uM): 0.06 |
| 638 | 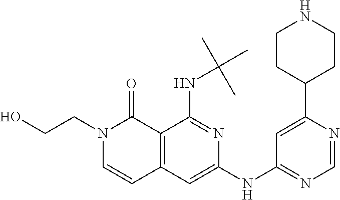 | ESI-MS m/z 438.3 (MH⁺). IC50 (uM): 0.096 |
| 639 | 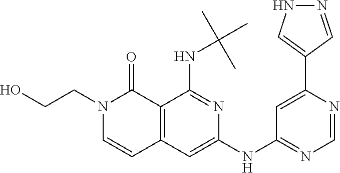 | ESI-MS m/z 421.2 (MH⁺). IC50 (uM): 0.285 |
| 640 | 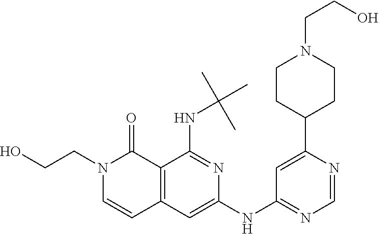 | ESI-MS m/z 482.3 (MH⁺). IC50 (uM): 0.116 |
| 641 | 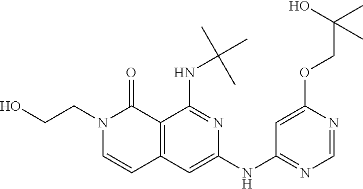 | ESI-MS m/z 443.2 (MH⁺). IC50 (uM): 0.056 |
| 642 | 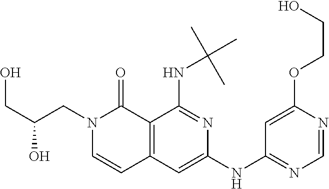 | ESI-MS m/z 445.2 (MH⁺). ¹H NMR (MeOD-d₄) [symbol] 9.64 (s, 1H), 8.27 (s, 1H), 7.18 (d, J = 7.2 Hz, 1H), 7.05 (s, 1H), 6.64 (s, 1H), 6.18 (d, J = 7.2 Hz, 1H), 4.28 (t, J = 5.2 Hz, 2H), 4.12 (dd, J = 4.0 Hz, 13.6 Hz, 1H), 3.86 (m, 1H), 3.78 (t, J = 5.2 Hz, 2H), 3.64 (dd, J = 4.0 Hz, 13.6 Hz, 1H), 3.45 (m, 2H), 1.47 (s, 9H). IC50 (uM): 0.029 |
| 643 | 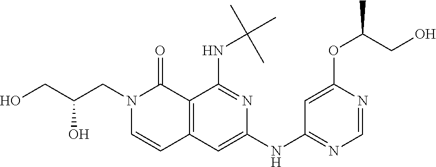 | ESI-MS m/z 459.2 (MH⁺). IC50 (uM): 0.009 |
| 644 | 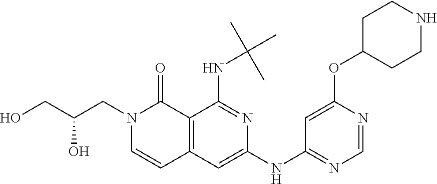 | ESI-MS m/z 483.3 (MH⁺). IC50 (uM): 0.004 |
| 645 | 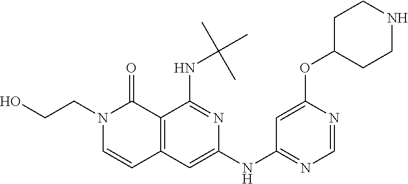 | ESI-MS m/z 554.3 (MH⁺). IC50 (uM): 0.005 |
| 646 | 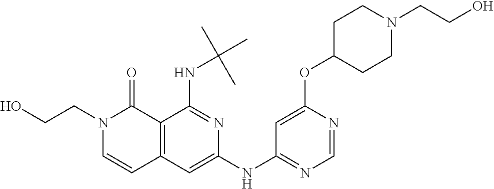 | ESI-MS m/z 498.3 (MH⁺). IC50 (uM): 0.009 |
| 647 | 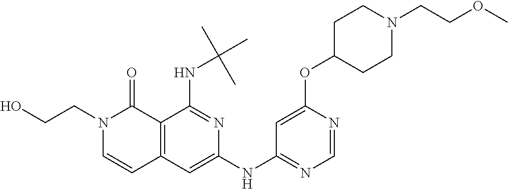 | ESI-MS m/z 512.3 (MH⁺). IC50 (uM): 0.014 |
| 648 | 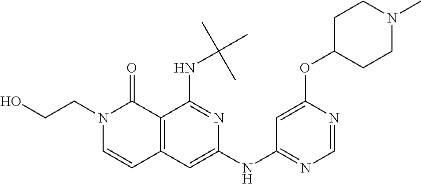 | ESI-MS m/z 468.3 (MH⁺). IC50 (uM): 0.006 |
| 649 | 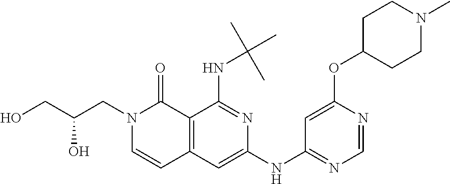 | ESI-MS m/z 498.3 (MH⁺). IC50 (uM): 0.006 |
| 650 | 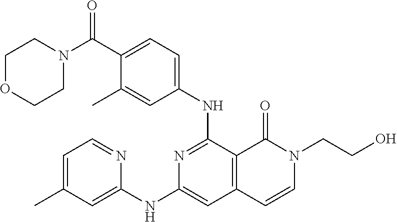 | ESI-MS m/z 515.4 (MH⁺). IC50 (uM): 0.186 |
| 651 | 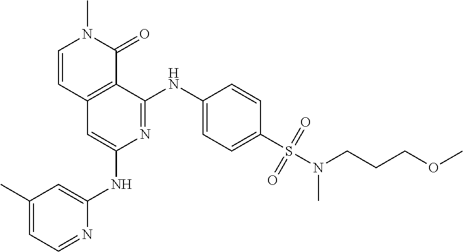 | ESI-MS m/z 523.2 (MH⁺). IC50 (uM): 2.405 |
| 652 | 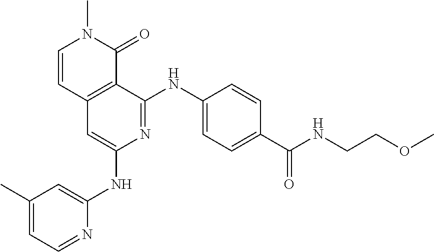 | ESI-MS m/z 459.3 (MH⁺). IC50 (uM): 0.66 |
| 653 | 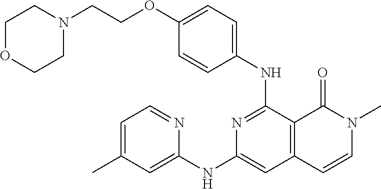 | ESI-MS m/z 487.1 (MH⁺). IC50 (uM): 0.333 |
| 654 | 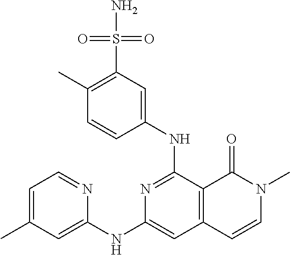 | ESI-MS m/z 451.4 (MH⁺). IC50 (uM): 0.25 |
| 655 | 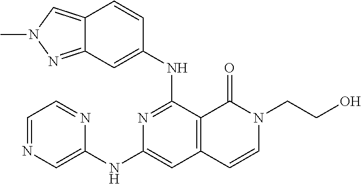 | ESI-MS m/z 421.1 (MH⁺). IC50 (uM): 0.139 |
| 656 | 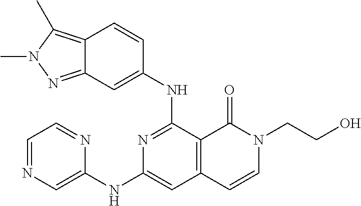 | ESI-MS m/z 443.1 (MH⁺). IC50 (uM): 0.168 |
| 657 | 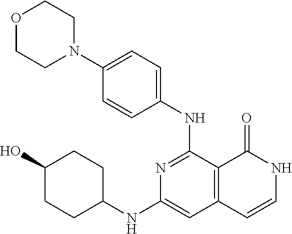 | ESI-MS m/z 436.5 (MH⁺). IC50 (uM): 0.152 |
| 658 | 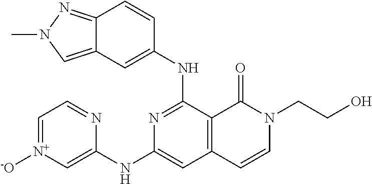 | ESI-MS m/z 445.2 (MH⁺). IC50 (uM): 0.099 |
| 659 | 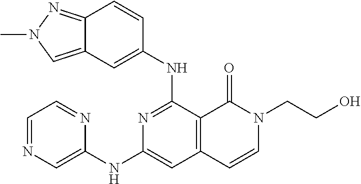 | ESI-MS m/z 429.1 (MH⁺). IC50 (uM): 0.103 |
| 660 | 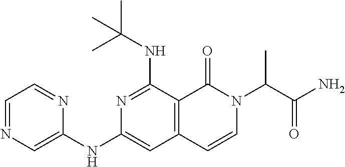 | ESI-MS m/z 382.2 (MH⁺). IC50 (uM): 0.013 |
| 661 | 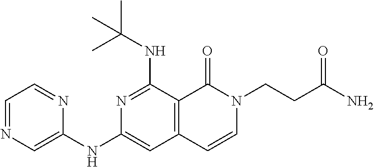 | ESI-MS m/z 382.1 (MH⁺). IC50 (uM): 0.01 |
| 662 | 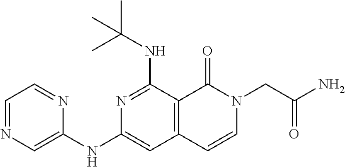 | ESI-MS m/z 368.1 (MH⁺). IC50 (uM): 0.017 |
| 663 | 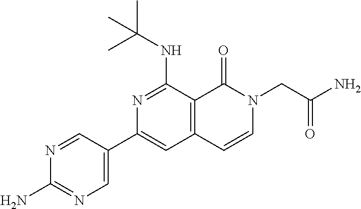 | ESI-MS m/z 368.2 (MH⁺). IC50 (uM): 0.043 |
| 664 | 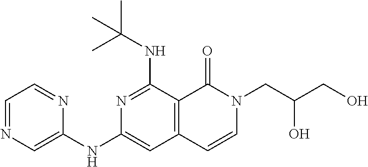 | ESI-MS m/z 385.2 (MH⁺). ¹H NMR (MeOH-d₄) δ 8.11 (d, J = 1.6 Hz, 1H), 7.45 (dd, J = 1.6, 2.8 Hz, 1H), 7.21 (d, J = 2.8 Hz, 1H), 6.45 (d, J = 7.6 Hz, 1H), 6.05 (s, 1H), 5.46 (d, J = 7.61 Hz, 1H), 3.40 (m, 1H), 3.16 (m, 1H), 2.91 (m, 1H), 2.75 (m, 2H), 2.51 (m, 1H), 1.75 (s, 9H). IC50 (uM): 0.007 |
| 665 | 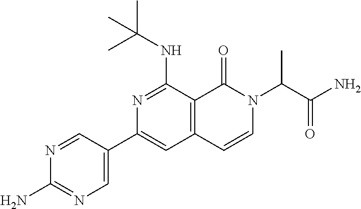 | ESI-MS m/z 382.2 (MH⁺). IC50 (uM): 0.005 |
| 666 | 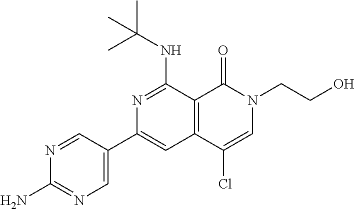 | ESI-MS m/z 389.1 (MH⁺). IC50 (uM): 0.028 |
| 667 | 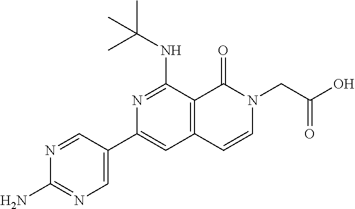 | ESI-MS m/z 369.1 (MH⁺). IC50 (uM): 0.023 |
| 668 | 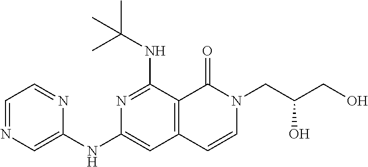 | ¹H NMR (MeOH-d₄) [symbol] 8.92 (d, J = 1.6 Hz, 1H), 8.23 (dd, J = 1.6, 2.8 Hz, 1H), 8.0 (d, J = 2.8 Hz, 1H), 7.25 (d, J = 7.6 Hz, 1H), 6.84 (s, 1H), 6.26 (d, J = 7.6 Hz, 1H), 4.21 (m, 1H), 3.97 (m, 1H), 3.71 (m, 1H), 3.55 (m, 2H), 3.16 (m, 1H), 1.56 (s, 9H). ESI-MS m/z 385.2 (MH⁺). IC50 (uM): 0.007 |
| 669 | 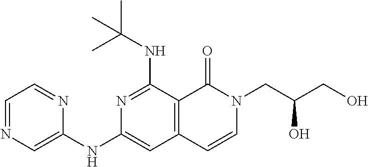 | ESI-MS m/z 385.2 (MH⁺). IC50 (uM): 0.006 |
| 670 | 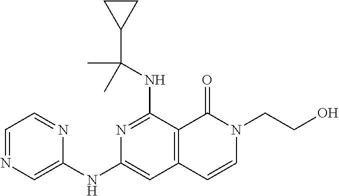 | ESI-MS m/z 381.2 (MH⁺). IC50 (uM): 0.089 |
| 671 | 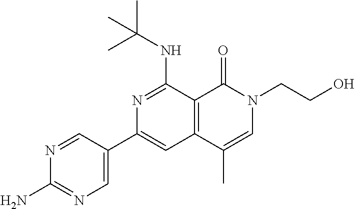 | ESI-MS m/z 369.2 (MH⁺). IC50 (uM): 0.013 |
| 672 | 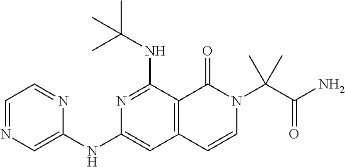 | ESI-MS m/z 396.2 (MH⁺). ¹H NMR (MeOH-d₄) δ 8.75 (m, 1H), 8.18 (dd, J = 1.2, 2.8 Hz, 1H), 7.95 (d, J = 2.8 Hz, 1H), 7.32 (d, J = 6.8 Hz, 1H), 7.04 (s, 1H), 6.63 (d, J = 7.2 Hz, 1H), 1.52 (s, 9H), 1.42 (s, 6H). IC50 (uM): 0.034 |
| 673 | 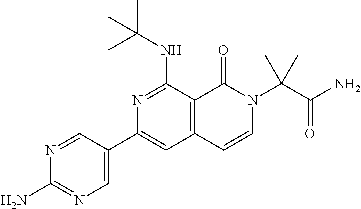 | ESI-MS m/z 396.2.2 (MH⁺). IC50 (uM): 0.057 |
| 674 | 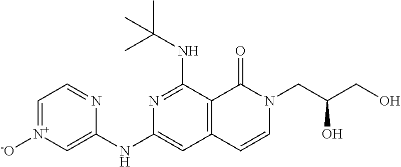 | ESI-MS m/z 401.2 (MH⁺). IC50 (uM): 0.007 |
| 675 | 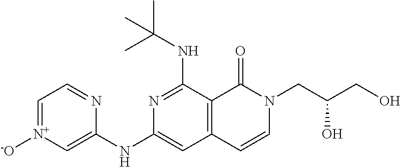 | ESI-MS m/z 401.2 (MH⁺). IC50 (uM): 0.006 |
| 676 | 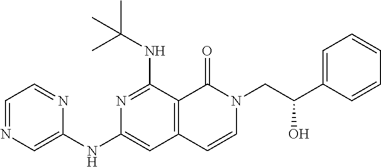 | ESI-MS m/z 431.2 (MH⁺). ¹H NMR (MeOH-d₄) δ 8.8 (s, 1H), 8.15 (m, 1H), 7.91 (d, J = 1.6 Hz, 1H), 7.35 (d, J = 7.2 Hz, 2H), 7.26 (t, J = 6.8 Hz, 2H), 7.19 (m, 1H), 7.05 (d, J = 7.2 Hz, 1H), 6.75 (s, 1H), 6.11 (d, J = 7.2 Hz, 1H), 4.93 (m, 1H), 4.10 (m, 1H), 3.71 (m, 1H), 1.48 (s, 9H). IC50 (uM): 0.12 |
| 677 | 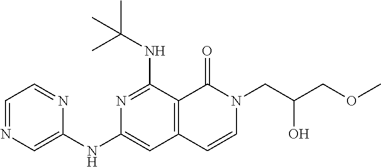 | ESI-MS m/z 399.2 (MH⁺). ¹H NMR (MeOH-d₄) δ 8.12 (d, J = 1.6 Hz, 1H), 7.36 (d, J = 7.2 J = 1.6, 2.8 Hz, 1H), 7.11 (d, J = 2.8 Hz, 1H), 6.35 (d, J = 7.6 Hz, 1H), 6.15 (s, 1H), 5.40 (d, J = 7.6 Hz, 1H), 3.42 (m, 1H), 3.35 (s, 3H), 3.11 (m, 1H), 2.92 (m, 1H), 2.70 (m, 2H), 2.53 (m, 1H), 1.57 (s, 9H). IC50 (uM): 0.012 |
| 678 | 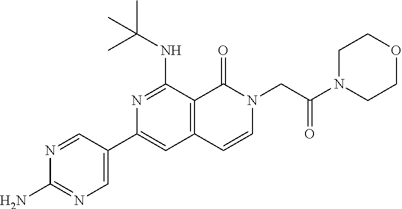 | ESI-MS m/z 438.2 (MH⁺). IC50 (uM): 0.078 |
| 679 | 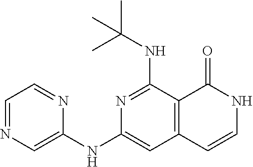 | ESI-MS m/z 311.2 (MH⁺). ¹H NMR (MeOH-d₄) δ 8.8 (m, 1H), 8.16 (dd, J = 1.2, 2.4 Hz, 1H), 7.91 (d, J = 2.4 Hz, 1H), 6.92 (d, J = 7.2 Hz, 1H), 6.76 (s, 1H), 6.18 (d, J = 7.2 Hz, 1H), 1.45 (s, 9H). IC50 (uM): 0.012 |
| 680 | 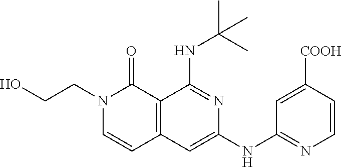 | ESI-MS m/z 398.2 (MH⁺). IC50 (uM): 0.643 |
| 681 | 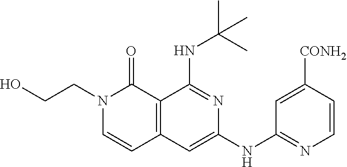 | ESI-MS m/z 397.2 (MH⁺). IC50 (uM): 0.082 |
| 682 | 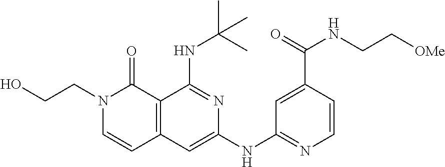 | ESI-MS m/z 455.2 (MH⁺). IC50 (uM): 0.329 |
| 683 | 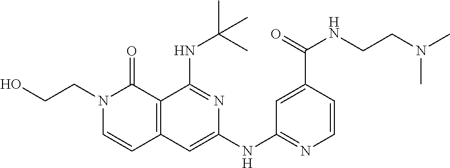 | ESI-MS m/z 468.3 (MH⁺). IC50 (uM): 0.595 |
| 684 | 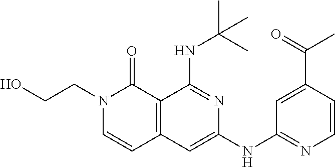 | ESI-MS m/z 396.2 (MH⁺). IC50 (uM): 0.051 |
| 685 | 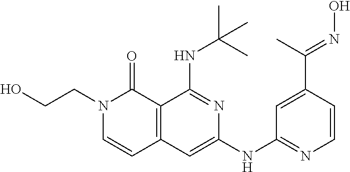 | ESI-MS m/z 411.2 (MH⁺). IC50 (uM): 0.062 |
| 686 | 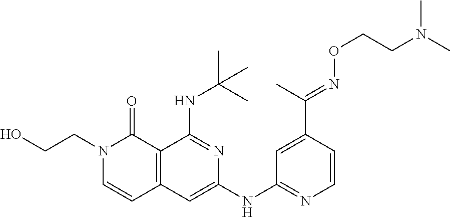 | ESI-MS m/z 482.3 (MH⁺). IC50 (uM): 0.246 |
| 687 | 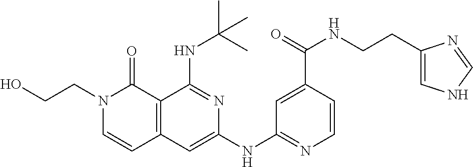 | ESI-MS m/z 491.2 (MH⁺). IC50 (uM): 0.248 |
| 688 | 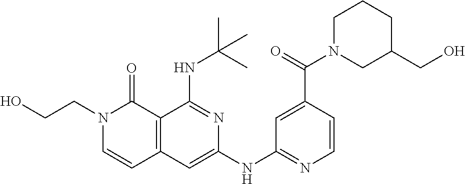 | ESI-MS m/z 495.3 (MH⁺). IC50 (uM): 0.866 |
| 689 | 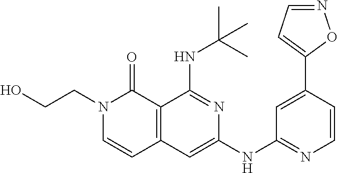 | ESI-MS m/z 421.2 (MH⁺). IC50 (uM): 0.151 |
| 690 | 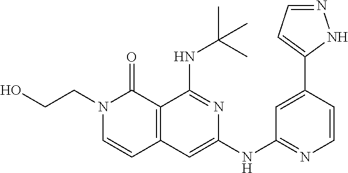 | ESI-MS m/z 42 IC50 (uM): 0.2 (MH⁺). IC50 (uM): 0.067 |
| 691 | 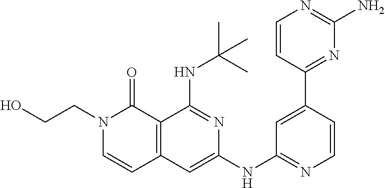 | ESI-MS m/z 447.2 (MH⁺). IC50 (uM): 0.059 |
| 692 | 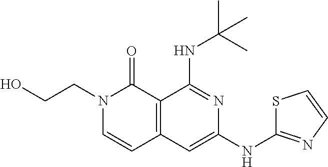 | ESI-MS m/z 36 IC50 (uM): 0.1 (MH⁺). IC50 (uM): 1.017 |
| 693 | 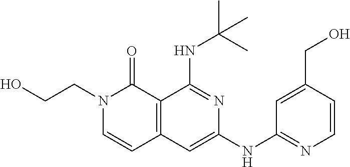 | ESI-MS m/z 384.2 (MH⁺). IC50 (uM): 0.042 |
| 694 | 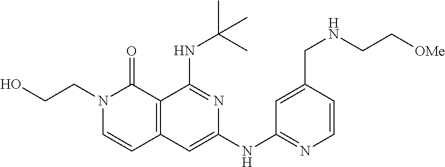 | ESI-MS m/z 441.3 (MH⁺). IC50 (uM): 0.237 |
| 695 | 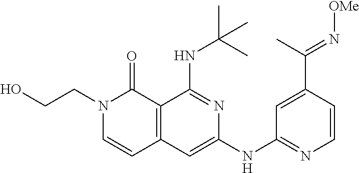 | ¹H NMR (CD₃OD, 400 MHz) δ 8.33 (d, J = 6.0 Hz, 1H), 7.61 (d, J = 7.2 Hz, 1H), 7.52 (d, J = 6.0 Hz, 1H), 7.37 (s, 1H), 6.45 (d, J = 7.2 Hz, 1H), 6.20 (s, 1H), 4.09 (t, J = 5.2 Hz, 2H) 4.06 (s, 3H), 3.84 (t, J = 5.2 Hz, 2H), 2.24 (s, 3H), 1.71 (s, 9H); ESI-MS m/z 425.2 (MH⁺). IC50 (uM): 0.431 |
| 696 | 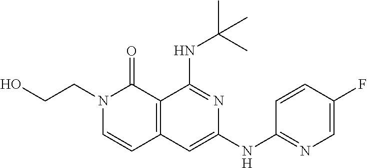 | ESI-MS m/z 372.2 (MH⁺). IC50 (uM): 0.328 |
| 697 | 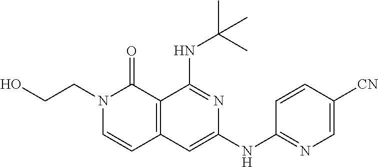 | ESI-MS m/z 379.2 (MH⁺). IC50 (uM): 0.208 |
| 698 | 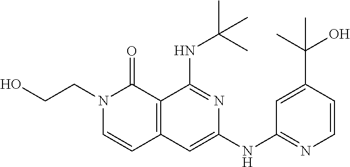 | ¹H NMR (CD₃OD, 400 MHz) δ 8.16 (d, J = 5.6 Hz, 1H), 7.49 (d, J = 1.6 Hz, 1H), 7.22 (d, J = 7.2 Hz, 1H), 7.03 (dd, J = 5.6, 1.6 Hz, 1H), 6.83 (s, 1H), 6.25 (d, J = 7.2 Hz, 1H), 3.98 (t, J = 5.2 Hz, 2H), 3.81 (t, J = 5.2 Hz, 2H), 1.55 (s, 9H), 1.53 (s, 6H); ESI-MS m/z 412.2 (MH⁺). IC50(uM): 0.131 |
| 699 | 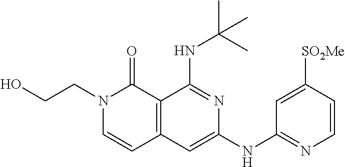 | ESI-MS m/z 432.2 (MH⁺). IC50 (uM): 0.099 |
| 700 | 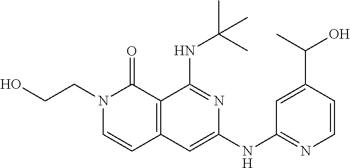 | ¹H NMR (CD₃OD, 400 MHz) δ 8.16 (d, J = 5.6 Hz, 1H), 7.69 (s, 1H), 7.23 (d, J = 7.6 Hz, 1H), 6.97 (d, J = 5.6 Hz, 1H), 6.59 (s, 1H), 6.24 (d, J = 7.6 Hz, 1H), 4.81 (q, J = 6.4 Hz, 1H), 3.99 (t, J = 5.2 Hz, 2H), 3.81 (t, J = 5.2 Hz, 2H), 1.57 (s, 9H), 1.45 (d, J = 6.4 Hz, 3H); ESI-MS m/z 398.2 (MH⁺). IC50 (uM): 0.012 |
| 701 | 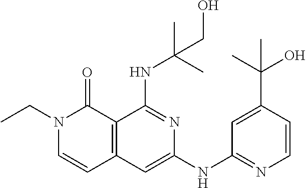 | ESI-MS m/z 412.2 (MH⁺). IC50 (uM): 0.069 |
| 702 | 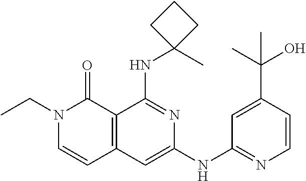 | ESI-MS m/z 408.2 (MH⁺). IC50 (uM): 0.085 |
| 703 | 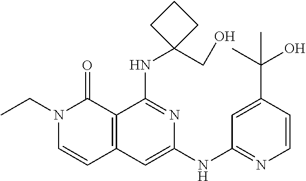 | ESI-MS m/z 424.2 (MH⁺). IC50 (uM): 0.085 |
| 704 | 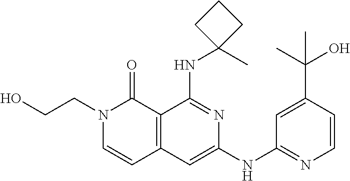 | ¹H NMR (CD₃OD, 400 MHz) δ 8.31 (d, J = 6.0 Hz, 1H), 7.56 (d, J = 7.2 Hz, 1H), 7.35 (s, 1H), 7.31 (d, J = 6.0 Hz, 1H), 6.42 (d, J = 7.2 Hz, 1H), 6.19 (s, 1H), 4.09 (t, J = 5.2 Hz, 2H), 3.84 (t, J = 5.2 Hz, 2H), 2.47 (t, J = 8.0 Hz, 4H), 2.12 (m, 2H), 1.79 (s, 3H), 1.55 (s, 6H); ESI-MS m/z 424.2 (MH⁺). IC50 (uM): 0.045 |
| 705 | 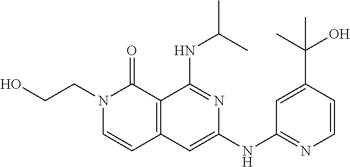 | ESI-MS m/z 398.2 (MH⁺). IC50 (uM): 0.014 |
| 706 | 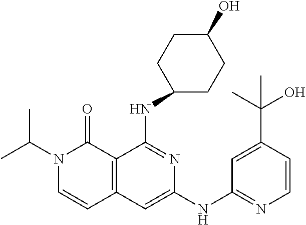 | ESI-MS m/z 452.3 (MH⁺). IC50 (uM): 0.055 |
| 707 | 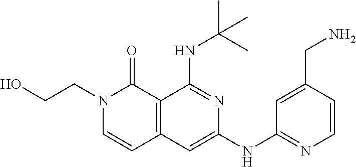 | ESI-MS m/z 383.2 (MH⁺). IC50 (uM): 0.059 |
| 708 | 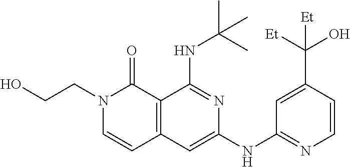 | ESI-MS m/z 44 IC50 (uM): 0.3 (MH⁺). IC50 (uM): 0.595 |
| 709 | 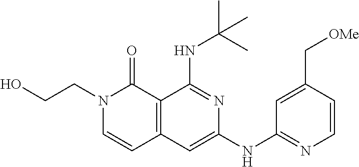 | ESI-MS m/z 398.2 (MH⁺). IC50 (uM): 0.011 |
| 710 | 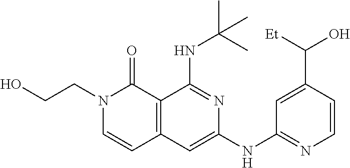 | ESI-MS m/z 412.2 (MH⁺). IC50 (uM): 0.037 |
| 711 | 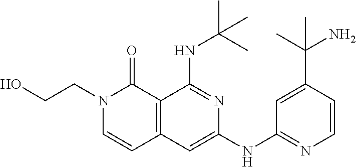 | ESI-MS m/z 411.2 (MH⁺). IC50 (uM): 0.197 |
| 712 | 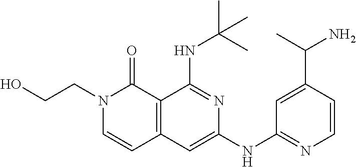 | ESI-MS m/z 397.2 (MH⁺). IC50 (uM): 0.053 |
| 713 | 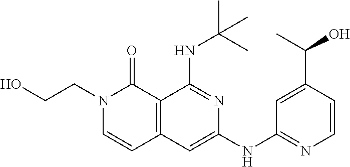 | ESI-MS m/z 398.2 (MH⁺). IC50 (uM): 0.003 |
| 714 | 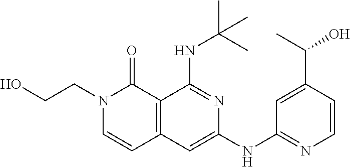 | ESI-MS m/z 398.2 (MH⁺). IC50 (uM): 0.013 |
| 715 | 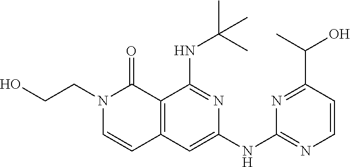 | ESI-MS m/z 399.2 (MH⁺). IC50 (uM): 0.679 |
| 716 | 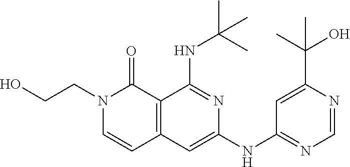 | ¹H NMR (CD₃OD, 400 MHz) δ 8.64 (d, J = 1.2 Hz, 1H), 7.69 (d, J = 1.2 Hz, 1H), 7.30 (d, J = 7.2 Hz, 1H), 7.12 (s, 1H), 6.32 (d, J = 7.2 Hz, 1H), 4.01 (t, J = 5.2 Hz, 2H), 3.82 (t, J = 5.2 Hz, 2H), 1.57 (s, 9H), 1.52 (s, 6H); ESI-MS m/z 413.2 (MH⁺). IC50 (uM): 0.035 |
| 717 | 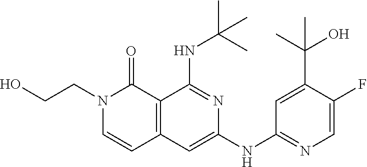 | ESI-MS m/z 43 IC50 (uM): 0.2 (MH⁺). IC50 (uM): 0.018 |
| 718 | 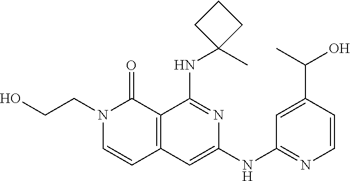 | ESI-MS m/z 41 IC50 (uM): 0.2 (MH⁺). IC50 (uM): 0.011 |
| 719 | 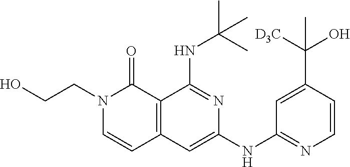 | ESI-MS m/z 401.2 (MH⁺). IC50 (uM): 0.017 |
| 720 | 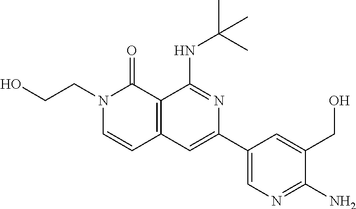 | ¹H NMR (CD₃OD, 400 MHz) δ 8.68 (d, J = 2.0 Hz, 1H), 8.31 (d, J = 2.0 Hz, 1H), 7.39 (d, J = 7.2 Hz, 1H), 6.96 (s, 1H), 6.43 (d, J = 7.2 Hz, 1H), 4.62 (s, 2H), 4.05 (t, J = 5.2 Hz, 2H), 3.84 (t, J = 5.2 Hz, 2H), 1.60 (s, 9H); ESI-MS m/z 384.2 (MH⁺). IC50 (uM): 0.075 |
| 721 | 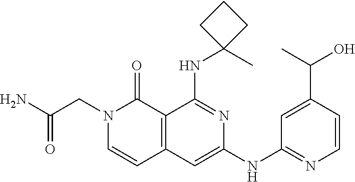 | ESI-MS m/z 423.2 (MH⁺). IC50 (uM): 0.018 |
| 722 | 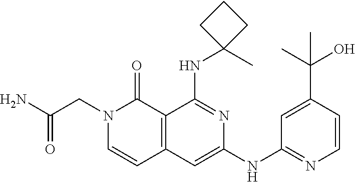 | ESI-MS m/z 437.2 (MH⁺). IC50 (uM): 0.155 |
| 723 | 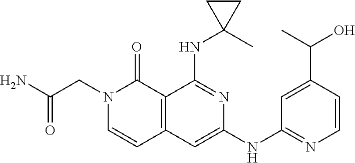 | ESI-MS m/z 409.2 (MH⁺). IC50 (uM): 0.048 |
| 724 | 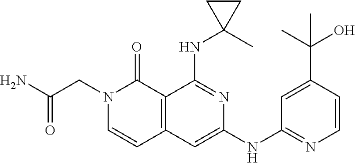 | ESI-MS m/z 423.2 (MH⁺). IC50 (uM): 0.203 |
| 725 | 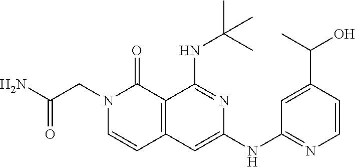 | ESI-MS m/z 411.2 (MH⁺). IC50 (uM): 0.031 |
| 726 | 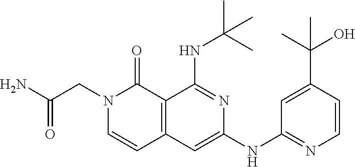 | ESI-MS m/z 425.2 (MH⁺). IC50 (uM): 0.108 |
| 727 | 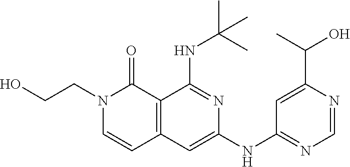 | ESI-MS m/z 399.2 (MH⁺). IC50 (uM): 0.03 |
| 728 | 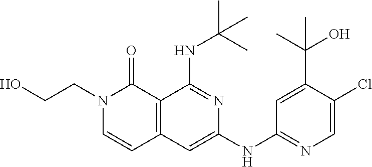 | ESI-MS m/z 446.2 (MH⁺). IC50 (uM): 0.283 |
| 729 | 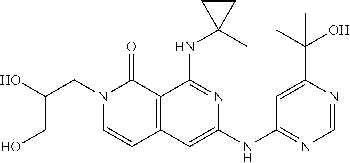 | ESI-MS m/z 441.2 (MH⁺). IC50 (uM): 0.031 |
| 730 | 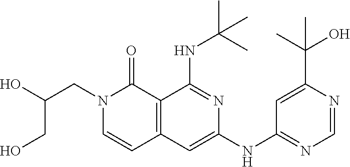 | ESI-MS m/z 443.2 (MH⁺). IC50 (uM): 0.042 |
| 731 | 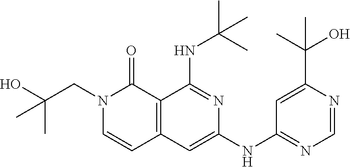 | ESI-MS m/z 441.3 (MH⁺). IC50 (uM): 0.109 |
| 732 | 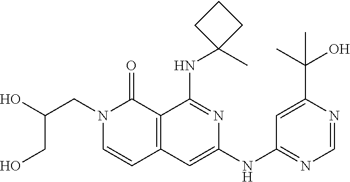 | ESI-MS m/z 455.2 (MH⁺). IC50 (uM): 0.035 |
| 733 | 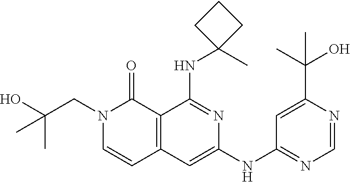 | ESI-MS m/z 453.3 (MH⁺). IC50 (uM): 0.092 |
| 734 | 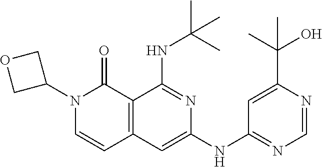 | ESI-MS m/z 425.2 (MH⁺). IC50 (uM): 0.057 |
| 735 | 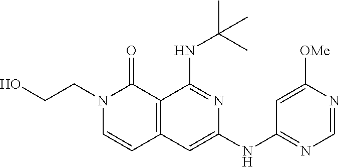 | ¹H NMR (CD₃OD, 400 MHz) δ 8.37 (s, 1H), 7.27 (d, J = 7.2 Hz, 1H), 7.25 (s, 1H), 6.62 (s, 1H), 6.27 (d, J = 7.2 Hz, 1H), 4.00 (t, J = 5.2 Hz, 2H), 3.94 (s, 3H), 3.81 (t, J = 5.2 Hz, 2H), 1.56 (s, 9H); ESI-MS m/z 385.2 (MH⁺). IC50 (uM): 0.05 |
| 736 | 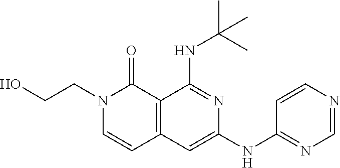 | ESI-MS m/z 355.2 (MH⁺). IC50 (uM): 0.09 |
| 737 | 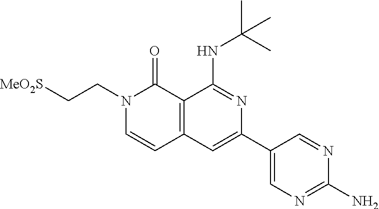 | ESI-MS m/z 417.2 (MH⁺). IC50 (uM): 0.02 |
| 738 | 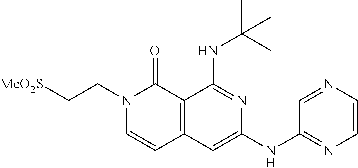 | ¹H NMR (CD₃OD, 400 MHz) δ 8.94 (s, 1H), 8.27 (d, J = 2.8 Hz, 1H), 8.03 (d, J = 2.8 Hz, 1H), 7.32 (d, J = 7.6 Hz, 1H), 6.87 (s, 1H), 6.30 (d, J = 7.6 Hz, 1H), 4.33 (t, J = 6.8 Hz, 2H), 3.60 (t, J = 6.8 Hz, 2H), 3.01 (s, 3H), 1.56 (s, 9H); ESI-MS m/z 417.2 (MH⁺). IC50 (uM): 0.009 |
| 739 | 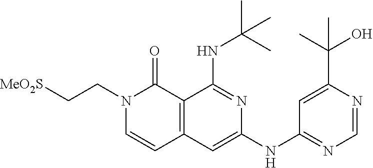 | ESI-MS m/z 475.2 (MH⁺). IC50 (uM): 0.035 |
| 740 | 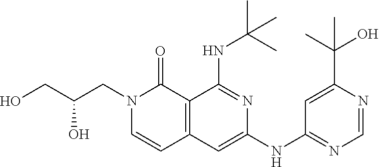 | ¹H NMR (CD₃OD, 400 MHz) δ 8.64 (d, J = 1.2 Hz, 1H), 7.69 (d, J = 1.2 Hz, 1H), 7.30 (d, J =7.2 Hz, 1H), 7.13 (s, 1H), 6.33 (d, J = 7.2 Hz, 1H), 4.23 (dd, J = 13.6, 4.0 Hz, 1H), 3.97 (m, 1H), 3.74 (dd, J = 13.6, 8.0 Hz, 1H), 3.55 (d, J = 4.8 Hz, 2H), 1.57 (s, 9H), 1.52 (s, 6H); ESI-MS m/z 443.2 (MH⁺). IC50 (uM): 0.054 |
| 741 | 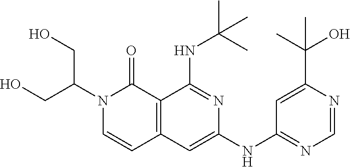 | ESI-MS m/z 443.2 (MH⁺). IC50 (uM): 0.0222 |
| 742 | 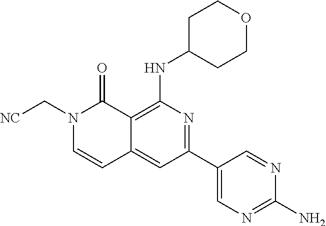 | ESI-MS m/z 377.8 (MH⁺). IC50 (uM): 0.034 |
| 743 | 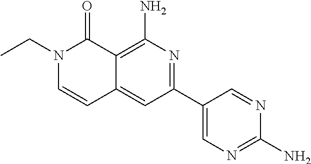 | ¹H NMR (CD₃OD) δ 8.73 (s, 2H), 7.93 (d, J = 7.2 Hz, 1H), 7.15 (s, 1H), 6.67 (d, J = 7.2 Hz, 1H), 4.11 (q, J = 7.2 Hz, 2H), 1.38 (t, J = 7.2 Hz, 3H); ESI-MS m/z 283.1 (MH⁺). IC50 (uM): 0.287 |
| 744 | 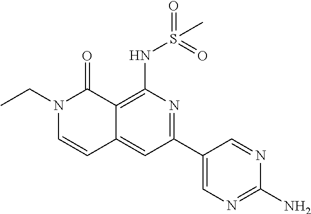 | ¹H NMR (400 MHz, CD₃OD) δ 9.09 (s, 2H), 6.38 (d, J = 7.2 Hz, 1H), 7.60 (s, 1H), 6.70 (d, J = 7.2 Hz, 1H), 4.08 (q, J = 7.6 Hz, 2H), 3.50 (s, 3H), 1.37 (t, J = 7.2 Hz, 3H); ESI-MS m/z 361.0 (MH⁺). IC50 (uM): 0.202 |
| 745 | 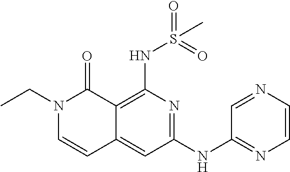 | ¹H NMR (CD₃OD) δ 9.11 (d, J = 1.2 Hz, 1H), 8.31 (dd, J = 1.6, 2.8 Hz, 1H), 7.47 (d, J = 7.6 Hz, 1H), 7.31 (s, 1H), 6.52 (d, J = 7.2 Hz, 1H), 4.01 (q, J = 7.2 Hz, 2H), 3.46 (s, 3H), 1.34 (t, J = 7.2 Hz, 3H); ESI-MS m/z 361.0 (MH⁺) IC50 (uM): 0.069 |
| 746 | 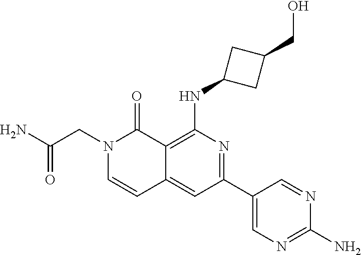 | ESI-MS m/z 396.1 (MH⁺). IC50 (uM): 0.044 |
| 747 | 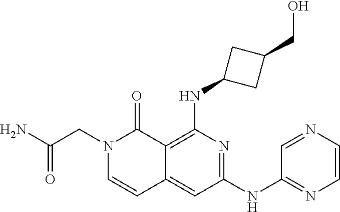 | ESI-MS m/z 396.1 (MH⁺). IC50 (uM): 0.017 |
| 748 | 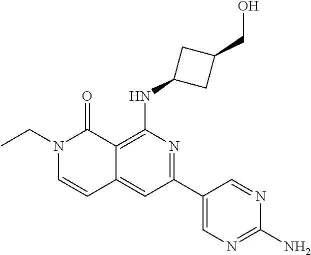 | ¹H NMR (CD₃OD) δ 9.46 (d, J = 6.4 Hz, 1H), 8.96 (s, 2H), 7.43 (d, J = 7.2 Hz, 1H), 6.94 (s, 1H), 6.46 (d, J = 7.6 Hz, 1H), 4.57 (m, 1H), 3.99 (dd, J = 7.2, 14.4 Hz, 2H), 3.51 (d, J = 6.0 Hz, 2H), 2.62 (qd, J = 9.2, 2.4 Hz, 2H), 2.34 (m, 1H), 1.75(qd, J = 9.2, 2.4 Hz, 2H), 1.34 (t, J = 7.2 Hz, 3H); ESI-MS m/z 367.1 (MH⁺). IC50 (uM): 0.007 |
| 749 | 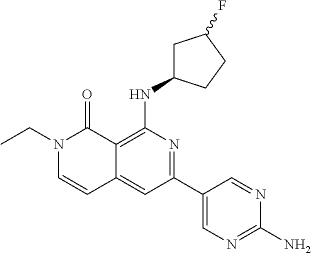 | ¹H NMR (CD₃OD) δ 8.99 (s, 2H), 7.62 (d, J = 7.2 Hz, 1H), 7.03 (s, 1H), 6.56 (d, J = 7.2 Hz, 1H), 5.25 (td, J = 4.8, 53.2 Hz, 1H), 4.68 (m, 1H), 4.04 (q, J = 7.2 Hz, 2H), 2.52-2.25 (m, 2H), 2.22-1.90 (m, 4H), 1.36 (t, J = 7.2 Hz, 3H); ESI-MS m/z 367.1 (MH⁺). IC50 (uM): 0.045 |
| 750 | 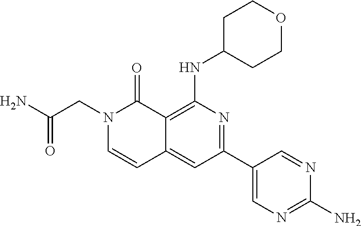 | ESI-MS m/z 395.8 (MH⁺). IC50 (uM): 0.024 |
| 751 | 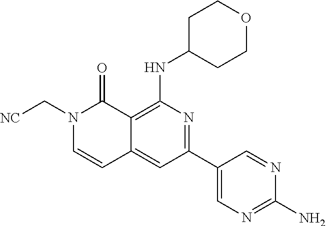 | ESI-MS m/z 377.9 (MH⁺). IC50 (uM): 0.07 |
| 752 | 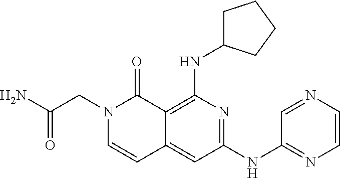 | ESI-MS m/z 379.9 (MH⁺). IC50 (uM): 0.018 |
| 753 | 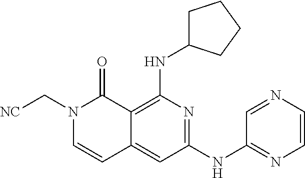 | ESI-MS m/z 361.9 (MH⁺). IC50 (uM): 0.027 |
| 754 | 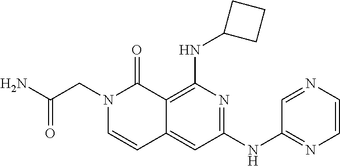 | ESI-MS m/z 365.9 (MH⁺). IC50 (uM): 0.012 |
| 755 | 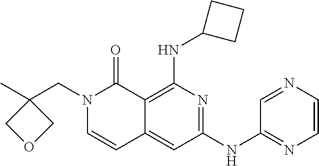 | ESI-MS m/z 393.2 (MH⁺). IC50 (uM): 0.009 |
| 756 | 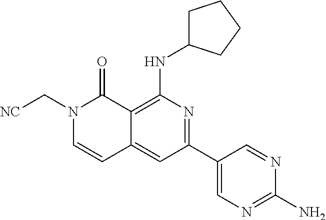 | ESI-MS m/z 362.1 (MH⁺). IC50 (uM): 0.117 |
| 757 | 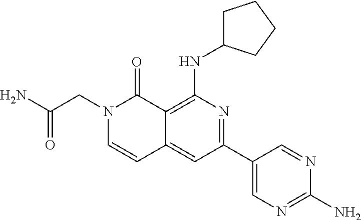 | ESI-MS m/z 38 IC50 (uM): 0.2 (MH⁺). IC50 (uM): 0.059 |
| 758 | 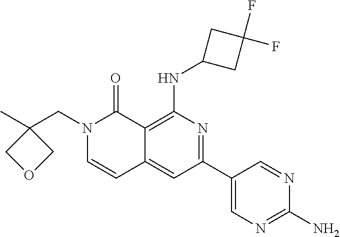 | ¹H NMR (CD₃OD) δ 9.09 (s, 2H), 7.82 (d, J = 7.2 Hz, 1H), 7.49 (s, 1H), 7.46 (d, J = 6.8 Hz, 1H), 4.94 (dd, J = 1.2, 11.2 Hz, 1H), 4.72 (d, J = 1 IC50 (uM): 0.8 Hz, 1H), 4.61 (m, 1H), 4.49 (d, J = 13.2 Hz, 1H), 4.25 (d, J = 13.6 Hz, 1H), 3.63 (s, 2H), 3.15 (m, 1H), 2.84 (m, 1H), 1.20 (s, 3H); ESI-MS m/z 429.2 (MH⁺). IC50 (uM): 0.09 |
| 759 | 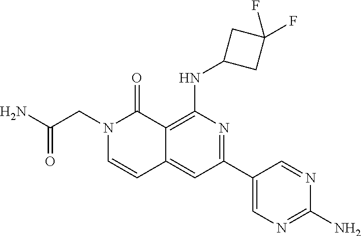 | ESI-MS m/z 402.1 (MH⁺). IC50 (uM): 0.175 |
| 760 | 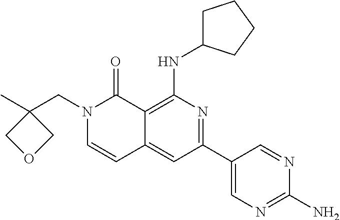 | ESI-MS m/z 406.9 (MH⁺). IC50 (uM): 0.128 |
| 761 | 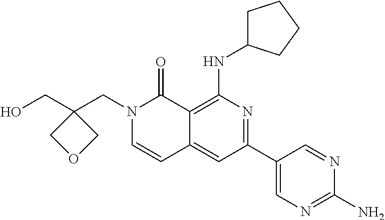 | ESI-MS m/z 423.2 (MH⁺). IC50 (uM): 0.066 |
| 762 | 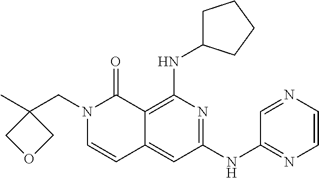 | ESI-MS m/z 407.2 (MH⁺). IC50 (uM): 0.022 |
| 763 | 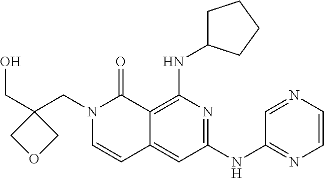 | ESI-MS m/z 423.1 (MH⁺). IC50 (uM): 0.012 |
| 764 | 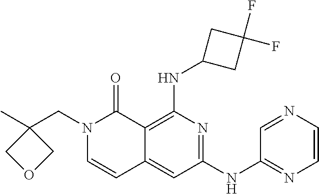 | ESI-MS m/z 345.1 (MH⁺). IC50 (uM): 0.027 |
| 765 | 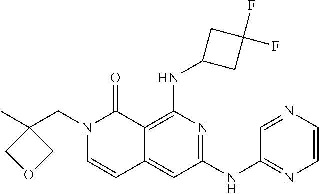 | ESI-MS m/z 429.2 (MH⁺). IC50 (uM): 0.052 |
| 766 | 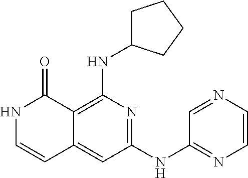 | ESI-MS m/z 323.2 (MH⁺). IC50 (uM): 0.007 |
| 767 | 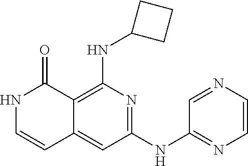 | ESI-MS m/z 309.1 (MH⁺). IC50 (uM): 0.003 |
| 768 | 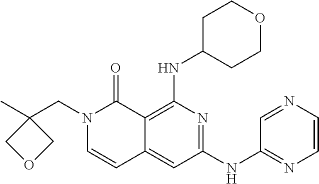 | ESI-MS m/z 309.1 (MH⁺). IC50 (uM): 0.045 |
| 769 | 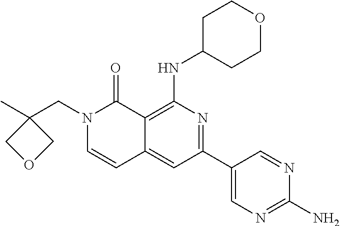 | ¹H NMR (CDCl₃) δ 9.41 (d, J = 7.2 Hz, 1H), 9.00 (s, 2H), 7.08 (d, J = 7.6 Hz, 1H), 6.79 (s, 1H), 6.34 (d, J = 7.2 Hz, 1H), 5.25 (s, 2H), 4.77 (d, J = 6.4 Hz, 2H), 4.4 (m, 1H), 4.38 (d, J = 6.4 Hz, 2H), 4.14 (s, 2H), 4.05 (dt, J = 12.0, 4.0 Hz, 2H), 3.63 (dt, J = 1.8, 11.6 Hz, 2H), 2.16 (m, 2H1), 1.73 (m, 2H), 1.42 (s, 3H); ESI-MS m/z 423.1 (MH⁺). IC50 (uM): 0.066 |
| 770 | 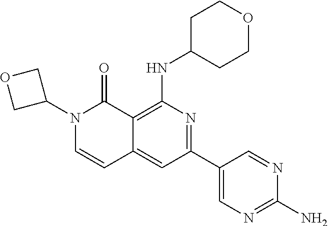 | ESI-MS m/z 395.1 (MH⁺). IC50 (uM): 0.036 |
| 771 | 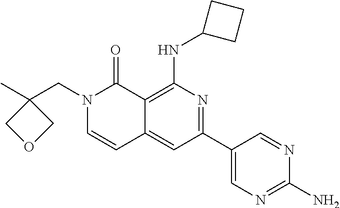 | ESI-MS m/z 393.2 (MH⁺). IC50 (uM): 0.075 |
| 772 | 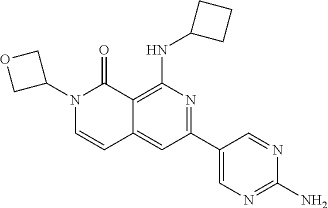 | ESI-MS m/z 365.1 (MH⁺). IC50 (uM): 0.025 |
| 773 | 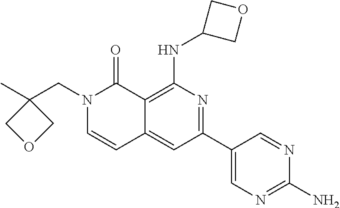 | ¹H NMR (CD₃OD) δ 8.94 (s, 1H), 7.88 (s, 1H), 7.43 (d, J = 4.8 Hz, 1H), 7.01 (s, 1H), 6.49 (d, J = 4.8 Hz, 1H), 5.25 (m, 1H), 5.07 (t, J = 5.2 Hz, 2H), 4.81 (d, J = 4.4 Hz, 2H), 4.70 Hz, 2H), 4.33 (d, J = 4.4 Hz, 2H), 4.18 (s, 2H), 1.37 (s, 3H); ESI-MS m/z 394.8 (MH⁺). IC50 (uM): 0.067 |
| 774 | 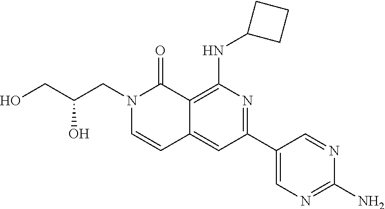 | ¹H NMR (CD₃OD) δ 8.98 (s, 2H), 7.43 (d, J = 7.2 Hz, 1H), 6.66 (d, J = 7.2 Hz, 1H), 4.70 (quintet, J = 8.0 Hz, 1H), 4.29 (dd, J = 3.6, 13.2 Hz, 1H), 4.00 (m, 1H), 3.79 (dd, J = 8.0, 13.6 Hz, 1H), 3.57 (m, 2H), 2.51 (m, 2H), 2.02 (m, 2H), 1.86 (m, 2H); ESI-MS m/z 383.1 (MH⁺). IC50 (uM): 0.034 |
| 775 | 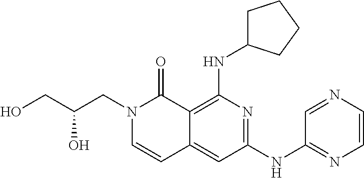 | ESI-MS m/z 397.2 (MH⁺). IC50 (uM): 0.01 |
| 776 | 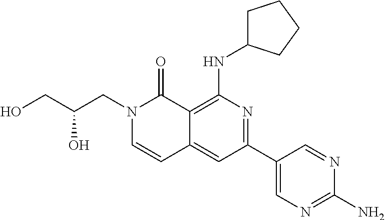 | ¹H NMR (CD₃OD) δ 8.99 (s, 2H), 7.42 (d, J = 7.2 Hz, 1H), 6.96 (s, 1H), 6.46 (d, J = 7.6 Hz, 1 H, 4.54 (quintet, J = 6.4 Hz, 1H), 4.28 (dd, J = 3.6, 13.6 Hz, 1H), 4.99 (m, 1H), 3.77 (dd, J = 8.0, 13.6 Hz, 1H), 3.57 (m, 2H), 2.14 (m, 1H), 1.82 (m, 2H), 1.72 (m, 2H), 1.63 (m, 2H); ESI-MS m/z 397.2 (MH⁺). IC50 (uM): 0.018 |
| 777 | 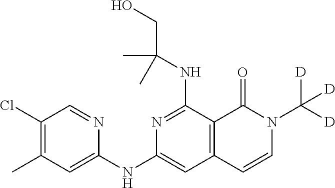 | ESI-MS m/z 391.20 (MH⁺). IC50 (uM): 0.137 |
| 778 | 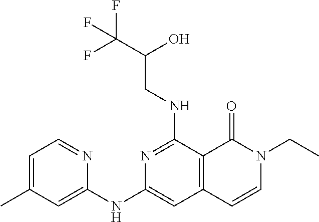 | ESI-MS m/z 408.20 (MH). IC50 (uM): 0.413 |
| 779 | 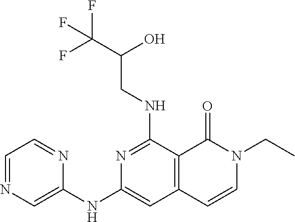 | ESI-MS m/z 395.20 (MH⁺). ¹H NMR (MeOD-d₄) δ 8.56 (s, 1H), 8.24 (m, 2H), 7.58 (d, J = 7.2 Hz, 1H), 6.45 (s, 1H), 6.42 (d, J = 7.2 Hz, 1H), 4.42 (m, IH), 4.02 (m, 1H), 3.99 (q, J = 6.8 Hz, 2H), 3.79 (m, 1H), 1.33 (t, J = 6.8 Hz, 3H). IC50 (uM): 0.145 |
| 780 | 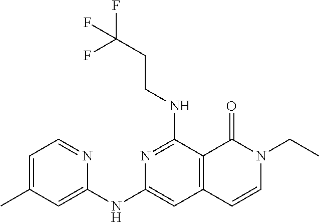 | ESI-MS m/z 392.20 (MH⁺). ¹H NMR (MeOD-d₄) δ 8.13 (d, J = 6.8 Hz, 1H), 7.50 (d, J = 7.2 Hz, 1H), 7.16 (d, J = 6.8 Hz, 1H), 7.15 (s, 1H), 6.43 (d, J = 7.2 Hz, 1H), 6.23 (s, 1H), 3.99 (q, J = 6.8 Hz, 2H), 3.91 (t, J = 6.8 Hz, 2H), 2.72 (m, 2H), 2.53 (s, 3H), 1.33 (t, J = 6.8 Hz, 3H). IC50 (uM): 0.862 |
| 781 | 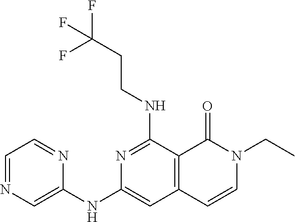 | ESI-MS m/z 379.20 (MH⁺). IC50 (uM): 0.299 |
| 782 | 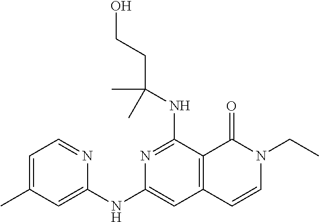 | ESI-MS m/z 394.30 (MH⁺). ¹H NMR (MeOD-d₄) δ 8.24 (d, J = 6.0 Hz, 1H), 7.58 (d, J = 7.2 Hz, 1H), 7.11 (d, J = 6.0 Hz, 1H), 7.02 (s, 1H), 6.43 (d, J = 7.2 Hz, 1H), 6.17 (s, 1H), 4.02 (q, J = 6.8 Hz, 2H), 3.77 (t, J = 6.8 Hz, 2H), 2.47 (s, 3H), 2.27 (t, J = 6.8 Hz, 2H), 1.67 (s, 6H), 1.34 (t, J = 6.8 Hz, 3H). IC50 (uM): 0.217 |
| 783 | 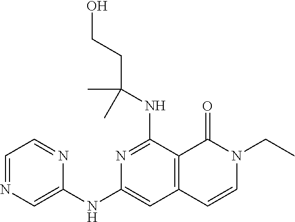 | ESI-MS m/z 369.20 (MH⁺). IC50 (uM): 0.179 |
| 784 | 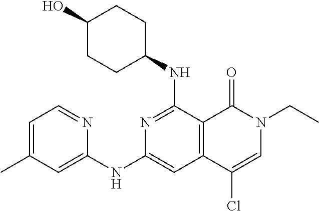 | ESI-MS m/z 414.20 (MH⁺). ¹H NMR (MeOD-d₄) δ 8.18 (d, J = 6.0 Hz, 1H), 7.83 (s, 1H), 7.17 (d, J = 6.0 Hz, 1H), 7.15 (s, 1H), 6.42 (s, 1H), 4.08 (m, 1H), 3.84 (m, 1H), 3.53 (s, 3H), 2.53 (s, 3H), 1.90 (m, 8H). IC50 (uM): 0.113 |
| 785 | 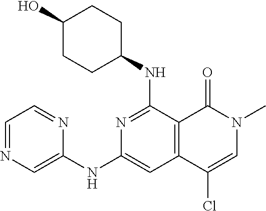 | ESI-MS m/z 401.20 (MH⁺). IC50 (uM): 0.144 |
| 786 | 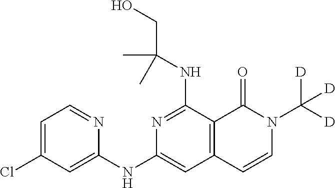 | ESI-MS m/z 377.20 (MH⁺). IC50 (uM): 0.071 |
| 787 | 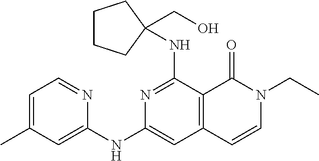 | ESI-MS m/z 414.20 (MH⁺). ¹H NMR (MeOD-d₄) δ 8.16 (d, J = 6.0 Hz, 1H), 7.53 (d, J = 7.2 Hz, 1H), 7.11 (d, J = 6.0 Hz, 1H), 7.04 (s, 1H), 6.43 (d, J = 7.2 Hz, 1H), 6.16 (s, 1H), 4.01 (q, J = 6.8 Hz, 2H), 3.90 (s, 3H), 2.50 (s, 3H), 2.18 (m, 2H), 2.08 (m, 2H), 1.81 (m, 4H), 1.33 (t, J = 6.8 Hz, 3H). IC50 (uM): 0.212 |
| 788 | 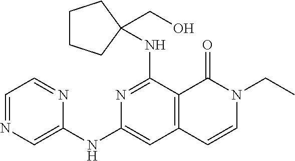 | ESI-MS m/z 381.20 (MH⁺). ¹H NMR (MeOD-d₄) δ 8.47 (s, 1H), 8.32 (d, J = 2.8 Hz, 1H), 8.27 (d, J = 2.8 Hz, 1H), 7.64 (d, J = 7.2 Hz, 1H), 6.47 (d, J = 7.2 Hz, 1H), 6.39 (s, 1H), 4.01 (q, J = 6.8 Hz, 2H), 3.90 (s, 3H), 2.18 (m, 4H), 1.86 (m, 4H), 1.34 (t, J = 6.8 Hz, 3H). IC50 (uM): 0.324 |
| 789 | 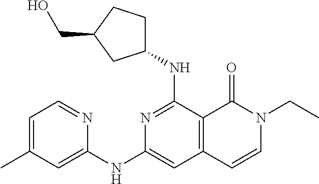 | ESI-MS m/z 394.30 (MH⁺). ¹H NMR (MeOD-d₄) δ 8.22 (d, J = 6.0 Hz, 1H), 7.52 (d, J = 7.2 Hz, 1H), 7.10 (d, J = 6.0 Hz, 1H), 7.05 (s, 1H), 6.40 (d, J = 7.2 Hz, 1H), 6.14 (s, 1H), 4.33 (m, 1H), 4.01 (q, J = 7.2 Hz, 2H), 3.59 (m, 2H), 2.48 (s, 3H), 2.43 (m, 1H), 2.33 (m, 1H), 2.03 (m, 1H), 1.98 (m, 2H), 1.79 (m, 1H), 1.60 (m, 1H), 1.34 (t, J = 7.2 Hz, 3H). IC50 (uM): 0.072 |
| 790 | 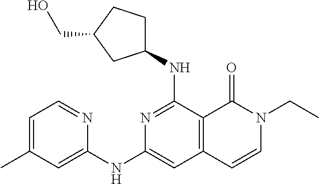 | ESI-MS m/z 394.30 (MH⁺). ¹H NMR (MeOD-d₄) δ 8.22 (d, J = 6.0 Hz, 1H), 7.52 (d, J = 7.2 Hz, 1H), 7.10 (d, J = 6.0 Hz, 1H), 7.05 (s, 1H), 6.40 (d, J = 7.2 Hz, 1H), 6.14 (s, 1H), 4.33 (m, 1H), 4.01 (q, J = 7.2 Hz, 2H), 3.59 (m, 2H), 2.48 (s, 3H), 2.43(m, 1H), 2.33 (m, 1H), 2.05 (m, 1H), 2.00 (m, 2H), 1.83 (m, 1H), 1.61 (m. 1H), 1.34 (t, J = 7.2 Hz, 3H). IC50 (uM): 0.088 |
| 791 | 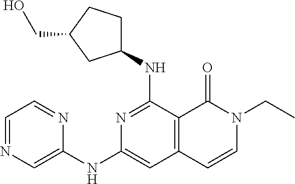 | ESI-MS m/z 381.20 (MH⁺). ¹H NMR (MeOD-d₄) δ 8.57 (s, 1H), 8.35 (d, J = 2.4 Hz, 1H), 8.30 (d, J = 2.4 Hz, 1H), 7.65 (d, J = 7.2 Hz, 1H), 6.48 (d, J = 7.2 Hz, 1H), 6.29 (s, 1H), 4.33 (m, 1H), 4.01 (q, J = 7.2 Hz, 2H), 3.59 (m, 2H), 2.43 (m, 2H), 2.07 (m, 3H), 1.87 (m, 1H), 1.63 (m, 2H), 1.34 (t, J = 7.2 Hz, 3H). IC50 (uM): 0.058 |
| 792 | 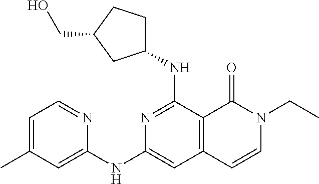 | ESI-MS m/z 394.30 (MH⁺). ¹H NMR (MeOD-d₄) δ 9.51 (d, J = 6.8 Hz, 1H), 8.04 (s, 1H), 7.22 (d, J = 7.2 Hz, 1H), 6.76 (d, J = 6.8 Hz, 1H), 6.30 (s, 1H), 6.23 (d, J = 7.2 Hz, 1H), 4.48 (m, 1H), 3.90 (q, J = 7.2 Hz,2H), 3.53 (m, 2H), 2.39 (s, 2H), 2.34 (s, 3H), 2.18 (m, 3H), 1.86 (m, 1H), 1.62 (m, 2H), 1.29 (t, J = 7.2 Hz, 3H). IC50 (uM): 0.033 |
| 793 | 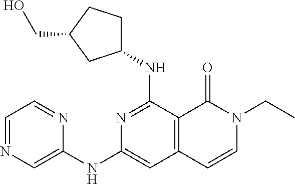 | ESI-MS m/z 381.20 (MH⁺). ¹H NMR (MeOD-d₄) δ 9.36 (s, 1H), 8.24 (d, J = 2.4 Hz, 1H), 8.02 (d, J = 2.4 Hz, 1H), 7.24 (d, J = 7.2 Hz, 1H), 6.46 (s, 1H), 6.26 (d, J = 7.2 Hz, 1H), 4.46 (m, 1H), 3.91 (q, J = 7.2 Hz, 2H), 3.54 (m, 2H), 2.40 (m, 1H), 2.25 (m, 1H), 2.15 (m, 1H), 1.88 (m, 1H), 1.67 (m, 1H), 1.57 (m, 1H), 1.34 (t, J = 7.2 Hz, 3H). IC50 (uM): 0.018 |
| 794 | 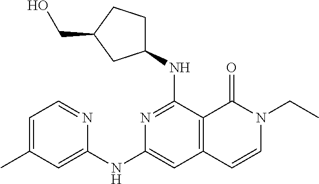 | ESI-MS m/z 394.30 (MH⁺). ¹H NMR (MeOD-d₄) δ 8.06 (d, J = 6.0 Hz, 1H), 7.43 (d, J = 7.2 Hz, 1H), 7.00 (d, J = 7.6 Hz, 1H), 6.94 (s, 1H), 6.31 (d, J = 7.6 Hz, 1H), 6.05 (s, 1H), 4.20 (m, 1H), 3.88 (q, J = 7.2 Hz, 2H), 3.48 (m, 2H), 2.41 (s, 3H), 2.40 (m, 1H), 2.28 (m, 1H), 2.15 (m, 1H), 1.88 (m, 1H), 1.78 (m, 1H), l.55 (m, 1H), 1.44 (m, 1H), 1.34 (t, J = 7.2 Hz, 3H). IC50 (uM): 0.094 |
| 795 | 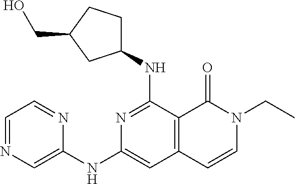 | ESI-MS m/z 381.20 (MH⁺). ¹H NMR (MeOD-d₄) δ 8.58 (s, 1H), 8.30 (s, 2H), 7.63 (d, J = 7.2 Hz, 1H), 6.47 (d, J = 7.2 Hz, 1H), 6.29 (s, 1H), 4.28 (m, 1H), 4.01 (q, J = 7.2 Hz, 2H), 3.58 (m, 2H), 2.56 (m, 1H), 2.39 (m, 3H), 2.27 (m, 1H), 2.00 (m, 2H), 1.67 (m, 2H), 1.58 (m, 2H), 1.34 (t, J = 7.2 Hz, 3H). IC50 (uM): 0.051 |
| 796 | 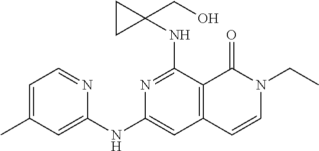 | ESI-MS m/z 366.20 (MH⁺). IC50 (uM): 0.216 |
| 797 | 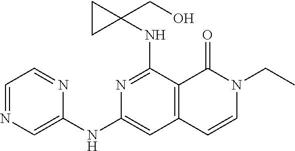 | ESI-MS m/z 353.20 (MH⁺). IC50 (uM): 0.106 |
| 798 | 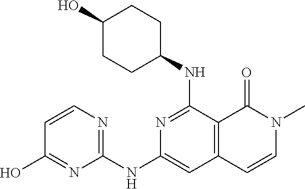 | ESI-MS m/z 383.20 (MH⁺). IC50 (uM): 0.128 |
| 799 | 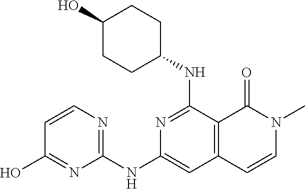 | ESI-MS m/z 383.20 (MH⁺). IC50 (uM): 0.029 |
| 800 | 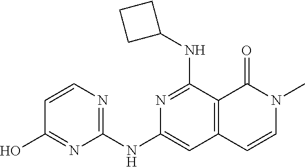 | ESI-MS m/z 339.20 (MH⁺). IC50 (uM): 0.009 |
| 801 | 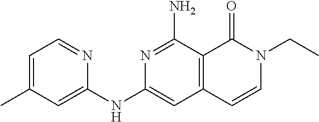 | ESI-MS m/z 296.20 (MH⁺). IC50 (uM): 1.9 |
| 802 | 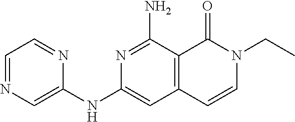 | ESI-MS m/z 283.20 (MH⁺). IC50 (uM): 1.18 |
| 803 | 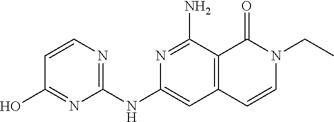 | ESI-MS m/z 299.10 (MH⁺). IC50 (uM): 0.307 |
| 804 | 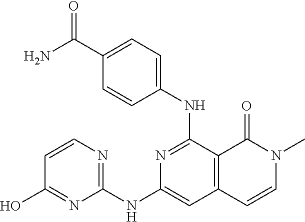 | ESI-MS m/z 404.20 (MH⁺). IC50 (uM): 0.194 |
| 805 | 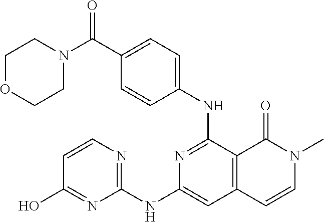 | ESI-MS m/z 474.20 (MH⁺). IC50 (uM): 0.752 |
| 806 | 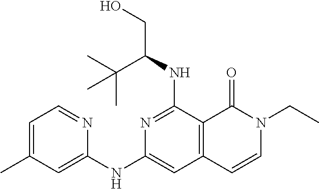 | ESI-MS m/z 396.30 (MH⁺). IC50 (uM): 0.568 |
| 807 | 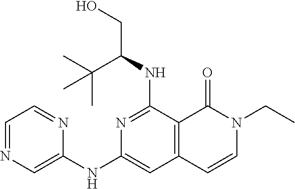 | ESI-MS m/z 383.30 (MH⁺). IC50 (uM): 0.216 |
| 808 | 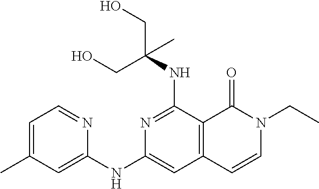 | ESI-MS m/z 384.20 (MH⁺). IC50 (uM): 0.081 |
| 809 | 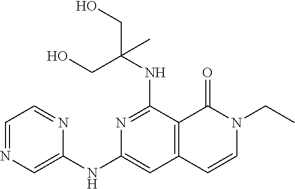 | ESI-MS m/z 371.20 (MH⁺). IC50 (uM): 0.153 |
| 810 | 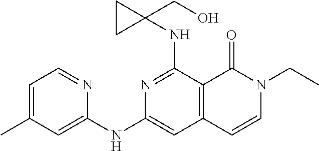 | ESI-MS m/z 38 IC50 (uM): 0.20 (MH⁺). IC50 (uM): 0.07 |
| 811 | 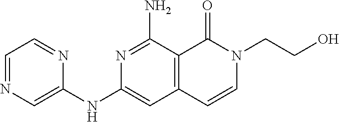 | ESI-MS m/z 299.20 (MH⁺). IC50 (uM): 0.651 |
| 812 | 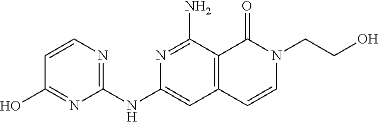 | ESI-MS m/z 315.20 (MH⁺). IC50 (uM): 0.359 |
| 813 | 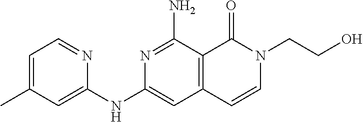 | ESI-MS m/z 312.20 (MH⁺). ¹H NMR (MeOD- d₄) δ 8.13 (d, J = 6.4 Hz, 1H), 7.45 (d, J = 7.2 Hz, 1H), 7.12 (d, J = 6.4 Hz, 1H), 7.06 (s, 1H), 6.35 (d, J = 7.2 Hz, 1H), 6.18 (s, 1H), 4.05 (t, J = 5.2 Hz, 2H), 3.83 (t, J = 5.2 Hz, 2H), 2.61 (s, 3H). IC50 (uM): 1.055 |
| 814 | 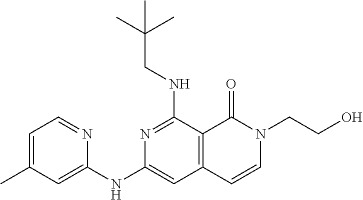 | ESI-MS m/z 382.30 (MH⁺). IC50 (uM): 0.39 |
| 815 | 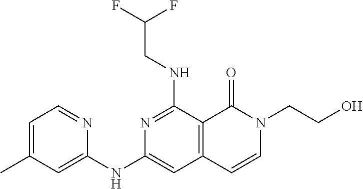 | ESI-MS m/z 376.20 (MH⁺). IC50 (uM): 0.117 |
| 816 | 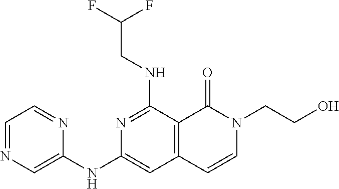 | ESI-MS m/z 366.20 (MH⁺). IC50 (uM): 0.066 |
| 817 | 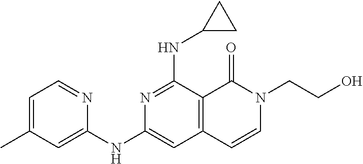 | ESI-MS m/z 352.20 (MH⁺). IC50 (uM): 0.108 |
| 818 | 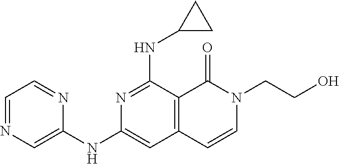 | ESI-MS m/z 339.20 (MH⁺). IC50 (uM): 0.027 |
| 819 | 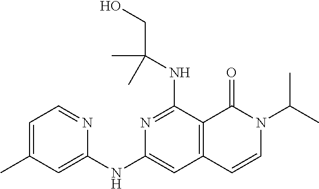 | ESI-MS m/z 382.20 (MH⁺). IC50 (uM): 0.062 |
| 820 | 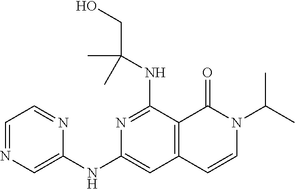 | ESI-MS m/z 369.20 (MH⁺). IC50 (uM): 0.084 |
| 821 | 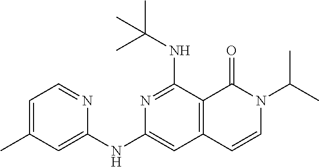 | ESI-MS m/z 366.30 (MH⁺). IC50 (uM): 0.272 |
| 822 | 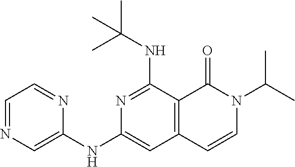 | ESI-MS m/z 353.20 (MH⁺). IC50 (uM): 0.165 |
| 823 | 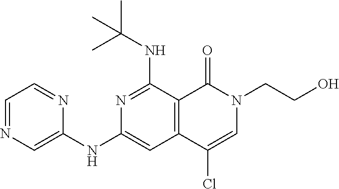 | ESI-MS m/z 389.20 (MH⁺). ¹H NMR (MeOD-d₄) δ 8.60 (s, 1H), 8.36 (s, 1H), 7.93 (s, 1H), 6.64 (s, 1H), (t, J = 5.2 Hz, 2H), 4.83 (t, J = 5.2 Hz, 2H), 1.69 (s, 9H). IC50 (uM): 0.682 |
| 824 | 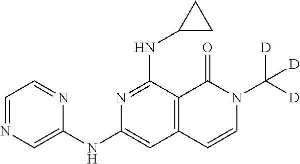 | ESI-MS m/z 312.20 (MH⁺). IC50 (uM): 0.018 |
| 825 | 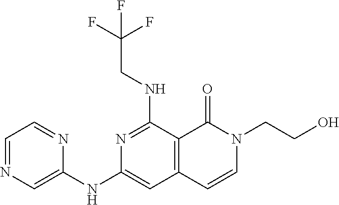 | ESI-MS m/z 381.20 (MH⁺). IC50 (uM): 0.022 |
| 826 | 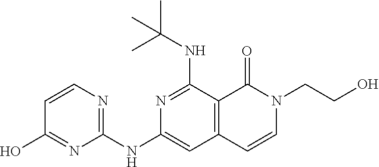 | ESI-MS m/z 371.20 (MH⁺). ¹H NMR (MeOD-d₄) δ 7.96 (d, J = 6.4 Hz, 1H), 732 (d, J = 7.2 Hz, 1H), 6.42 (s, IH), 6.27 (d, J = 7.2 Hz, 1H), 5.93 (d, J = 6.4 Hz, 1H), 4.01 (t, J = 52 Hz, 2H), 3.81 (t, J = 5.2 Hz, 2H), 1.58 (s, 9H). IC50 (uM): 0.025 |
| 827 | 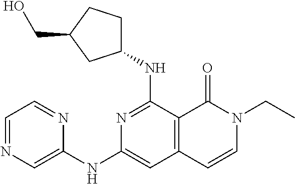 | ESI-MS m/z 381.20 (MH⁺). ¹H NMR (MeOD-d₄) δ 8.53 (s, 1H), 8.35 (d, J = 2.0 Hz, 1H), 8.31 (d, J = 2.0 Hz, 1H), 7.67 (d, J = 7.2 Hz, 1H), 6.48 (d, J = 7.2 Hz, 1H), 6.29 (s, 1H), 4.31 (m, 1H), 4.01 (q, J = 7.2 Hz, 2H), 3.59 (m, 2H), 2.46 (m, 2H), 2.07 (m, 3H), 1.87 (m, 1H), 1.63 (m, 2H), 1.34 (t, J = 7.2 Hz, 3H). IC50 (uM): 0.012 |
| 828 | 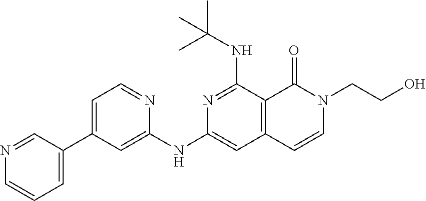 | ESI-MS m/z 431.20 (MH⁺). IC50 (uM): 0.072 |
| 829 | 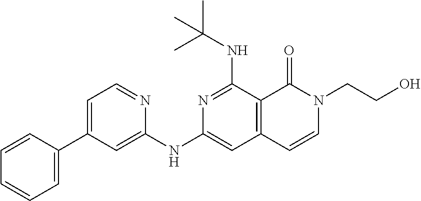 | ESI-MS m/z 430.30 (MH⁺). IC50 (uM): 0.271 |
| 830 | 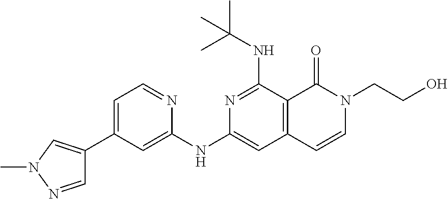 | ESI-MS m/z 434.30 (MH⁺). IC50 (uM): 0.103 |
| 831 | 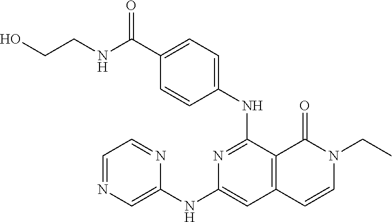 | ESI-MS m/z 446.20 (MH⁺). ¹H NMR (MeOD-d₄) δ 8.93 (d, J = 7.6 Hz, 1H), 8.24 (s, 1H), 8.01 (s, IH), 7.85 (d, J = 6.8 Hz, 2H), 7.76 (d, J = 6.8 Hz, 2H), 7.15 (d, J = 7.6 Hz, 1H), 7.01 (d, J = 7.2 Hz, 1H), 6.37 (d, J = 7.2 Hz, 1H), 3.97 (q, J = 6.8 Hz, 2H), 3.73 (t, J = 5.2 Hz, 2H), 3.52 (q, J = 5.2 Hz, 2H), 1.36 (t, J = 6.8 Hz, 2H). IC50 (uM): 0.213 |
| 832 | 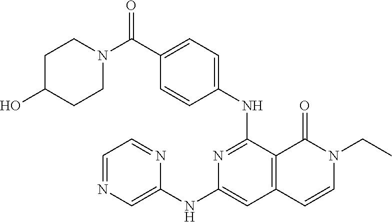 | ESI-MS m/z 486.30 (MH⁺). IC50 (uM): 1.13 |
| 833 | 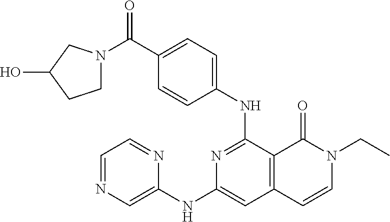 | ESI-MS m/z 472.20 (MH⁺). ¹H NMR (MeOD-d₄) δ 8.64 (d, J = 23.6 Hz, 1H), 8.23 (s, 1H), 8.00 (s, 1H), 7.89 (d, J = 14.4 Hz, 2H), 7.52 (d, J = 14.4 Hz, 2H), 7.21 (d, J = 23.6 Hz, 1H), 7.16 (d, J = 7.2 Hz, 1H), 6.39 (d, J =7.2 Hz, 1H), 4.48 (m, 1H), 3-99 (q, J = 7.2 Hz, 2H), 3.47-3.84 (m, 4H), 1.93-2.07 (m, 2H), 1.36 (t, J = 7.2 Hz, 2H). IC50 (uM): 0.263 |
| 834 | 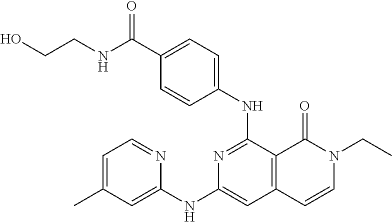 | ESI-MS m/z 459.20 (MH⁺). ¹H NMR (MeOD-d₄) δ 7.89 (d, J = 8.4 Hz, 2H), 7.62 (d, J = 8.4 Hz, 2H), 7.59 (d, J = 6.0 Hz, 1H), 7.45 (d, J = 7.2 Hz, 1H), 7.08 (s, 1H), 6.97 (d, J = 6.0 Hz, 1H), 6.48 (s, 1H), 6.37 (d, J = 7.2 Hz, 1H), 3.99 (q, J = 6.8 Hz, 2H), 3.77 (t, J = 4.8 Hz, 2H), 3.57 (t, J = 4.8 Hz, 2H), 2.43 (s, 3H), 1.36 (t, J = 6.8 Hz, 2H). IC50 (uM): 0.126 |
| 835 | 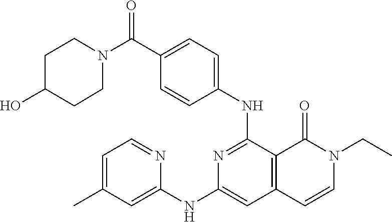 | ESI-MS m/z 499.30 (MH⁺). IC50 (uM): 0.131 |
| 836 | 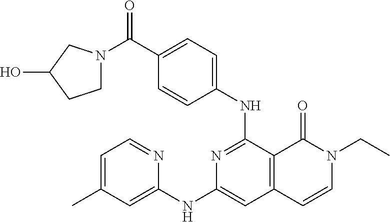 | ESI-MS m/z 485.20 (MH⁺). IC50 (uM): 0.119 |
| 837 | 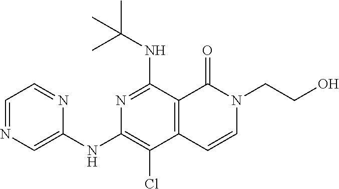 | ESI-MS m/z 389.20 (MH⁺). IC50 (uM): 1.063 |
| 838 | 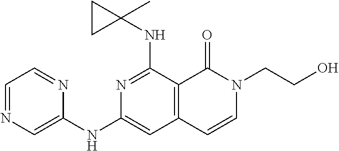 | ESI-MS m/z 353.20 (MH⁺). ¹H NMR (MeOD-d₄) δ 9.85 (d, J = 1.6 Hz, 1H), 9.60 (s, 1H), 8.17 (s, 1H), 8.03 (d, J = 1.6 Hz, 1H), 7.11 (d, J = 3.2 Hz, 1H), 6.20 (d, J = 3.2 Hz, 1H), 3.96 (t, J = 6.4 Hz, 2H), 3.80 (t, J = 6.4 Hz, 2H), 1.50 (s, 3H), IC50 (uM): 0.84 (m, 4H). IC50 (uM): 0.017 |
| 839 | 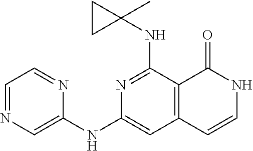 | ESI-MS m/z 309.20 (MH⁺). IC50 (uM): 0.008 |
| 840 | 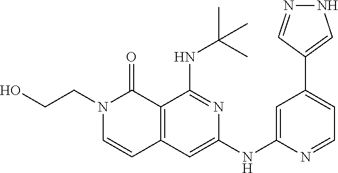 | ESI-MS m/z 42 IC50 (uM): 0.2 (MH⁺). IC50 (uM): 0.117 |
| 841 | 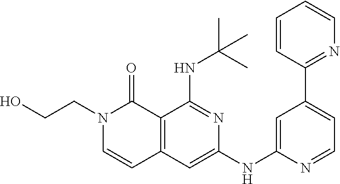 | ¹H NMR (MeOD) δ 8.79 (d, J = 4.8 Hz, 1H), 8.47 (d, J = 5.6 Hz, 1H), 8.09 (m, 2H), 7.80 (m 2H), 7.63 (d, J = 7.2 Hz, 1H), 7.59 (m, 1H), 6.47 (d, J= 7.2 Hz, 1H), 6.23 (s, 1H), 4.09 (m, 2H), 3.84 (m, 2H), 1.73 (s, 9H). ESI-MS m/z 431.2 (MH⁺). IC50 (uM): 0.291 |
| 842 | 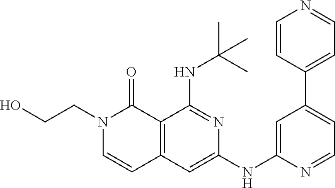 | ESI-MS m/z 431.2 (MH⁺). IC50 (uM): 0.051 |
| 843 | 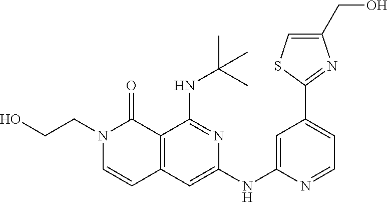 | ¹H NMR (MeOD) δ 9.67 (s, 1H), 8.32 (d, J = 5.6 Hz, 1H), 8.04 (s, 1H), 7.54 (s, 1H), 7.40 (d, J = 5.6 Hz, 1H), 7.24 (d, J = 7.2 Hz, 1H), 6.78 (s, 1H), 6.27 (d, J = 7.2 Hz, 1H), 4.78 (s, 2H), 3.98 (m, 2H), 3.82 (m, 2H), 1.54 (s, 9H). ESI-MS m/z 467.2 (MH⁺). IC50 (uM): 0.082 |
| 844 | 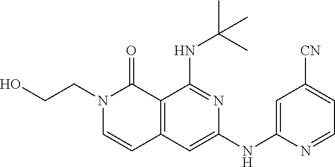 | ESI-MS m/z 439.2 (MH⁺). IC50 (uM): 0.007 |
| 845 | 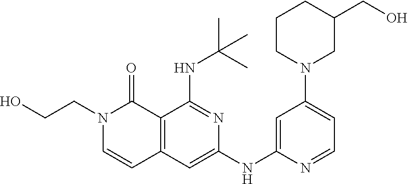 | ESI-MS m/z 467.3 (MH⁺). IC50 (uM): 0.766 |
| 846 | 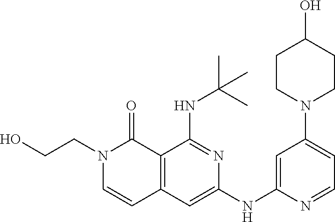 | ESI-MS m/z 453.3 (MH⁺). IC50 (uM): 0.216 |
| 847 | 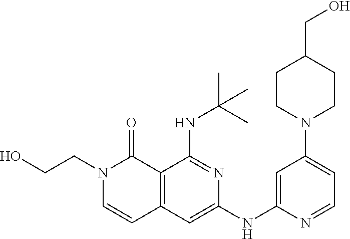 | ESI-MS m/z 467.3 (MH⁺). IC50 (uM): 0.5 |
| 848 | 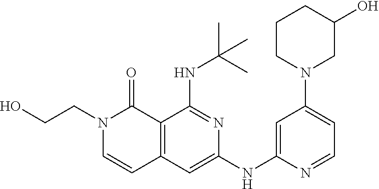 | ESI-MS m/z 453.3 (MH⁺). IC50 (uM): 0.615 |
| 849 | 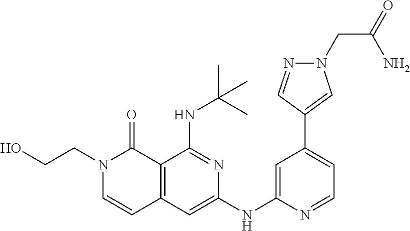 | ESI-MS m/z 477.2 (MH⁺). IC50 (uM): 0.102 |
| 850 | 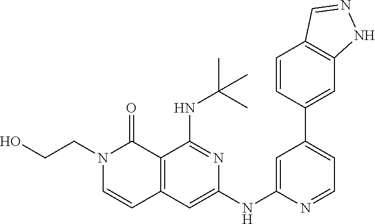 | ESI-MS m/z 47 IC50 (uM): 0.2 (MH⁺). IC50 (uM): 0.559 |
| 851 | 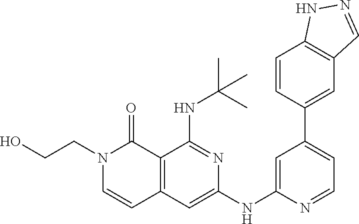 | ESI-MS m/z 47 IC50 (uM): 0.2 (MH⁺). IC50 (uM): 0.222 |
| 852 | 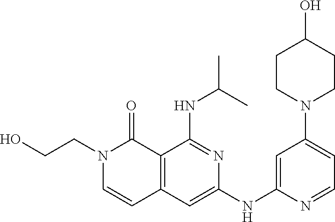 | ESI-MS m/z 439.2 (MH⁺). IC50 (uM): 0.3 |
| 853 | 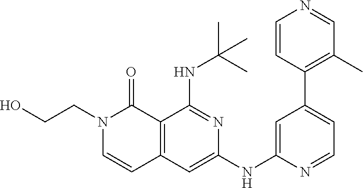 | ESI-MS m/z 445.2 (MH⁺). IC50 (uM): 0.111 |
| 854 | 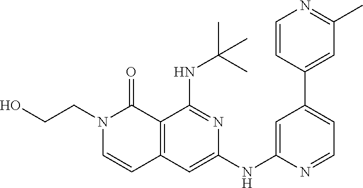 | ESI-MS m/z 445.2 (MH⁺). IC50 (uM): 0.064 |
| 855 | 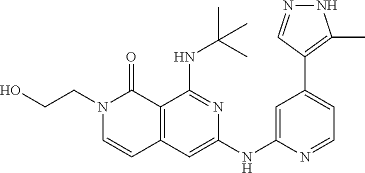 | ESI-MS m/z 434.2 (MH⁺). IC50 (uM): 0.244 |
| 856 | 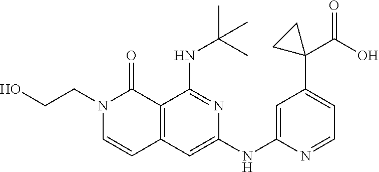 | ESI-MS m/z 438.2 (MH⁺). IC50 (uM): 0.393 |
| 857 | 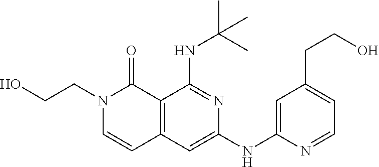 | ESI-MS m/z 398.2 (MH⁺). IC50 (uM): 0.004 |
| 858 | 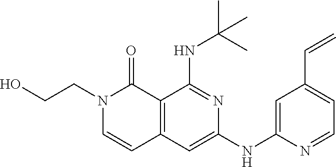 | ESI-MS m/z 48 IC50 (uM): 0.2 (MH⁺). IC50 (uM): 0.046 |
| 859 | 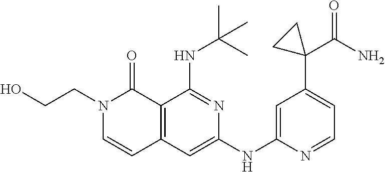 | ESI-MS m/z 437.2 (MH⁺). IC50 (uM): 0.17 |
| 860 | 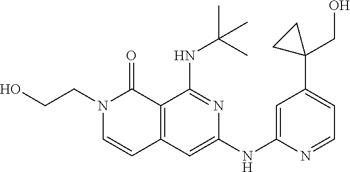 | ESI-MS m/z 424.2 (MH⁺). IC50 (uM): 0.028 |
| 861 | 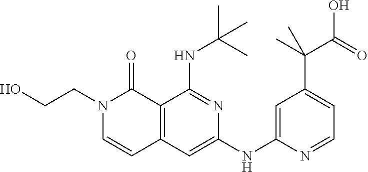 | ESI-MS m/z 44 IC50 (uM): 0.2 (MH⁺). IC50 (uM): 0.169 |
| 862 | 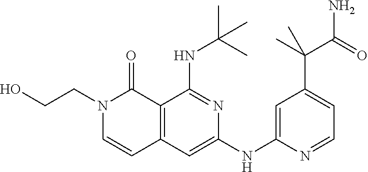 | ESI-MS m/z 439.2 (MH⁺). IC50 (uM): 0.579 |
| 863 | 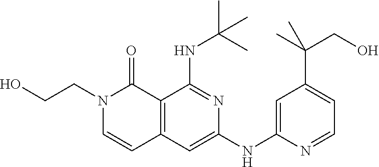 | ESI-MS m/z 426.2 (MH⁺). IC50 (uM): 0.301 |
| 864 | 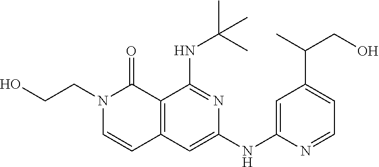 | ESI-MS m/z 412.2 (MH⁺). IC50 (uM): 0.002 |
| 865 | 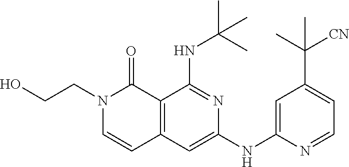 | ESI-MS m/z 421.2 (MH⁺). |
| 866 | 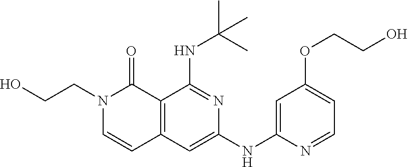 | ESI-MS m/z 414.2 (MH⁺). IC50 (uM): 0.011 |
| 867 | 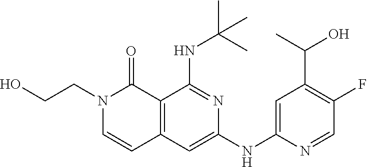 | ESI-MS m/z 322.16 (MH⁺). |
| 868 | 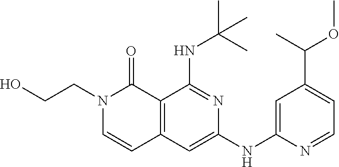 | ¹H NMR (CDCl₃) δ 9.66 (s, 1H), 8.23 (d, J = 5.2 Hz, 1H), 7.59 (s, 1H), 7.01 (d, J = 7.2 Hz, 1H), 6.84 (d, J = 5.2 Hz, 1H), 6.51 (s, 1H), 6.19 (d, J = 7.2 Hz, 1H), 4.27 (m, 1H), 4.04 (m, 2H), 3.95 (m, 2H), 3.27 (s, 3H), 1.56 (s, 9H), 1.43 (d, 3H). ESI-MS m/z 412.2 (MH⁺). IC50 (uM): 0.016 |
| 869 | 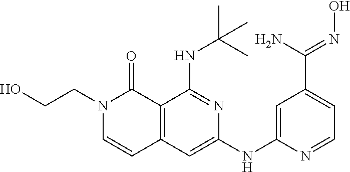 | ESI-MS m/z 412.2 (MH⁺). IC50 (uM): 0.006 |
| 870 | 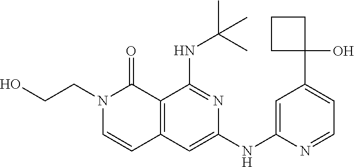 | ¹H NMR (CDCl₃) δ 9.586 (s, 1H), 8.27 (d, J = 5.2 Hz, 1H), 7.7.42 (s, 1H), 7.01 (d, J = 7.2 Hz, 1H), 7.18 (b, 1H), 7.00 (m, 2H), , 6.79 (s, 1H), 6.22 (d, J = 7.2 Hz, 1H), 4.04 (m, 2H), 3.95 (m, 2H), 2.54 (m, 2H), 2.38 (m, 2H), 2.05-2.15 (m, 1H), 1.70- 1.85 (m, 1H), 1.56 (s, 9H). ESI- MS m/z 424.2 (MH⁺). IC50 (uM): 0.076 |
| 871 | 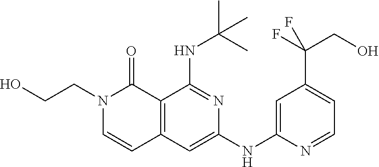 | ¹H NMR (MeOD) δ 8.30 (d, J = 5.2 Hz, 1H), 7.97 (s, 1H), 7.22 (d, J = 7.2 Hz, 1H), 7.01 (d, J = 5.2 Hz, 1H), 6.59 (s, 1H), 6.24 (d, J = 7.2 Hz, 1H), 3.98 (m, 2H), 3.90 (t, J = 13.2 Hz, 2H), 3.81 (m, 2H), 1.54 (s, 9H). ESI-MS m/z 434.2 (MH⁺) IC50 (uM): <0.001269 |
| 872 | 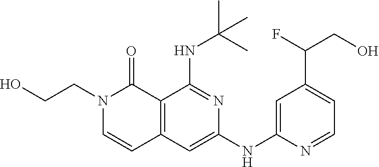 | ESI-MS m/z 416.2 (MH⁺). IC50 (uM): <0.001165 |
| 873 | 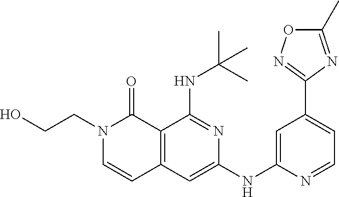 | ESI-MS m/z 436.2 (MH⁺). IC50 (uM): 0.027 |
| 874 | 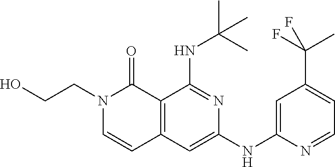 | ESI-MS m/z 418.2 (MH⁺). IC50 (uM): 0.033 |
| 875 | 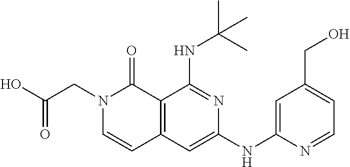 | ESI-MS m/z 398.2 (MH⁺). IC50 (uM): 0.006 |
| 876 | 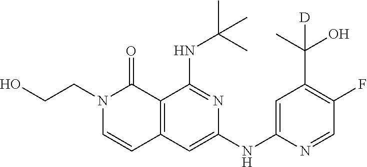 | ESI-MS m/z 417.2 (MH⁺). IC50 (uM): 0.015 |
| 877 | 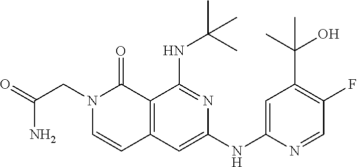 | ESI-MS m/z 443.2 (MH⁺). IC50 (uM): 0.094 |
| 878 | 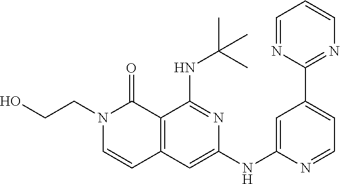 | ESI-MS m/z 432.2 (MH⁺). IC50 (uM): 0.009 |
| 879 | 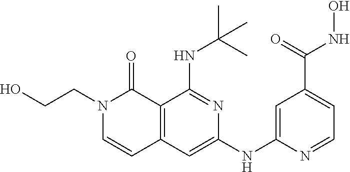 | ESI-MS m/z 413.2 (MH⁺). IC50 (uM): 0.026 |
| 880 | 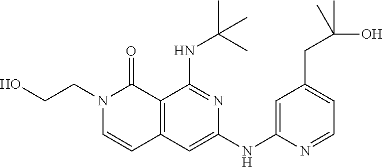 | ESI-MS m/z 426.2 (MH⁺). IC50 (uM): 0.074 |
| 881 | 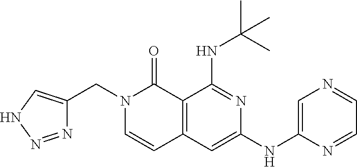 | ¹H NMR (MeOD) δ 8.88 (s, 1H), 8.22 (s, 1H), 8.00 (s, 1H), 7.81 (b, 1H), 7.38 (d, J = 5.2 Hz, 1H), 6.79 (b, 1H), 6.27 (d, J= 7.2 Hz, 1H), 5.17 (s, 2H), 1.54 (s, 9H). ESI-MS m/z 392.2 (MH⁺). IC50 (uM): 0.018 |
| 882 | 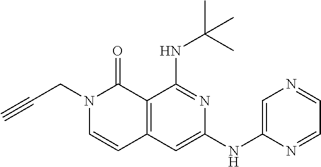 | ESI-MS m/z 349.2 (MH⁺). IC50 (uM): 0.051 |
| 883 | 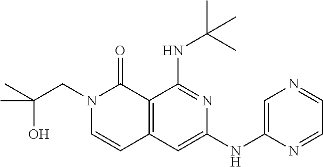 | ESI-MS m/z 382.2 (MH⁺). IC50 (uM): 0.038 |
| 884 | 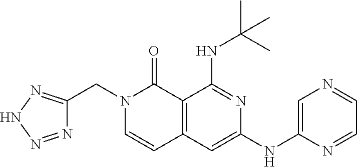 | ESI-MS m/z 393.2 (MH⁺). IC50 (uM): 0.027 |
| 885 | 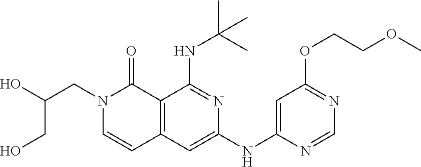 | ESI-MS m/z 459.2 (MH⁺). IC50 (uM): 0.078 |
| 886 | 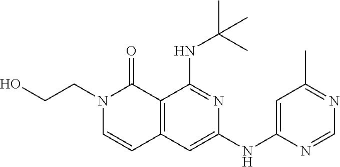 | ESI-MS m/z 369.2 (MH⁺). IC50 (uM): 0.06 |
| 887 | 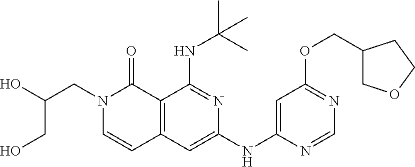 | ESI-MS m/z 485.2 (MH⁺). IC50 (uM): 0.048 |
| 888 | 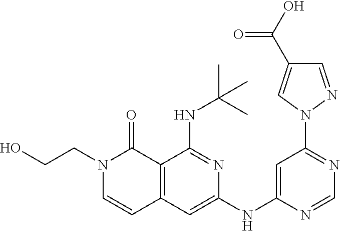 | ESI-MS m/z 465.2 (MH⁺). IC50 (uM): 0.024 |
| 889 | 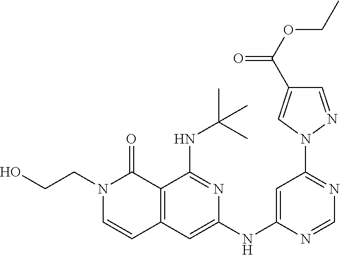 | ESI-MS m/z 493.2 (MH⁺). IC50 (uM): 2.185 |
| 890 | 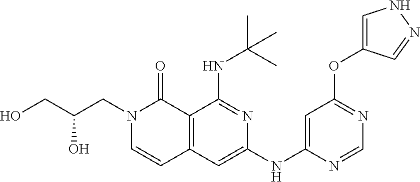 | ESI-MS m/z 467.2 (MH⁺). IC50 (uM): 0.021 |
| 891 | 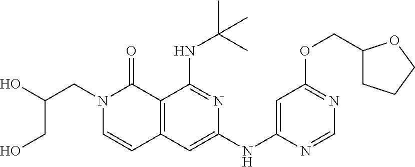 | 1H NMR (CDCl₃) δ 9.55 (b, 1H), 8.43 (s, 1H), 7.23 (b, 1H), 7.08 (d, J = 7.2 Hz, 1H), 6.92 (s, 1H), 6.49 (s, 1H), 6.26 (d, J= 7.2 Hz, 1H), 4.45 (m, 1H), 4.30 (m, 2H), 4.00-4.10 (m, 2H), 3.92 (m, 1H), 3.82 (m, 1H), 3.59 (b, 2H), 3.15 (b, H), 1.90- 2.10 (m, 3H), 1.75 (m, 1H), 1.54 (s, 9H). ESI-MS m/z 485.2 (MH⁺). IC50 (uM): 0.06 |
| 892 | 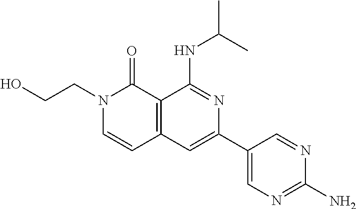 | ESI-MS m/z 341.10 (MH⁺). IC50 (uM): 0.136 |
| 893 | 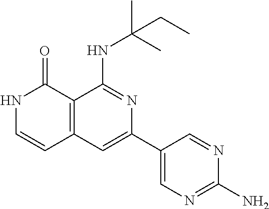 | ESI-MS m/z 325.10 (MH⁺). IC50 (uM): 0.043 |
| 894 | 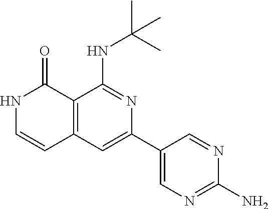 | ESI-MS m/z 311.10 (MH⁺). ¹H NMR (DMSO-d₆) δ 11.33 (s, 1H), 9.59 (s, 1H), 8.92 (s, 2H), 7.25 (d, J = 6.8 Hz, 1H), 7.07 (s, 1H), 7.03 (s, 2H), 6.32 (d, J = 6.8 Hz, 1H), 1.51 (s, 9H). IC50 (uM): 0.005 |
| 895 | 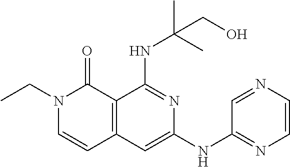 | ESI-MS m/z 354.80 (MH⁺). IC50 (uM): 0.027 |
| 896 | 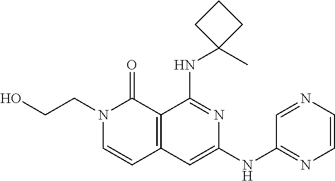 | ESI-MS m/z 366.80 (MH⁺). IC50 (uM): 0.011 |
| 897 | 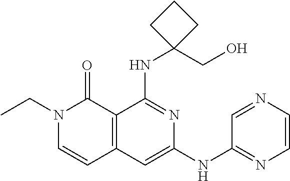 | ESI-MS m/z 366.70 (MH⁺). IC50 (uM): 0.005 |
| 898 | 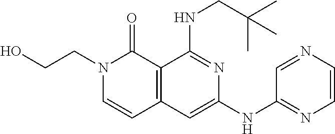 | ESI-MS m/z 369.10 (MH⁺). IC50 (uM): 0.02 |
| 899 | 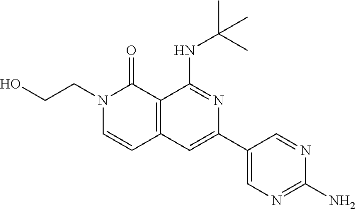 | ESI-MS m/z 355.10 (MH⁺). ¹H NMR (DMSO-d₆) δ 9.68 (s, 1H), 8.93 (s, 2H), 7.49 (d, J = 7.2 Hz, 1H), 7.07 (s, 1H), 7.03 (s, 2H), 6.36 (d, J = 7.2 Hz, 1H), 4.88 (t, J = 5.6 Hz, 1H), 3.95 (m, 2H), 3.64 (m, 2H), 1.52 (s, 9H). IC50 (uM): 0.017 |
| 900 | 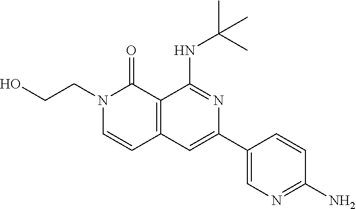 | ESI-MS m/z 354.10 (MH⁺). IC50 (uM): 1.024 |
| 901 | 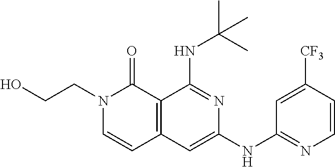 | ESI-MS m/z 422.10 (MH⁺). IC50 (uM): 0.231 |
| 902 | 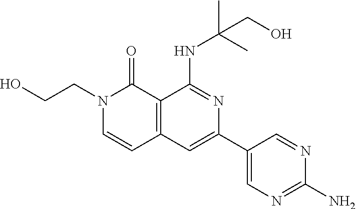 | ESI-MS m/z 371.10 (MH⁺). IC50 (uM): 0.057 |
| 903 | 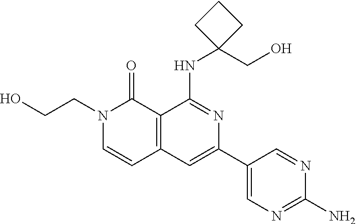 | ESI-MS m/z 383.10 (MH⁺). ¹H NMR (DMSO-d₆) δ 9.60 (s, 1H), 8.89 (s, 2H), 7.50 (d, J = 7.2 Hz, 1H), 7.07 (s, 1H), 7.02 (s, 2H), 6.36 (d, J = 7.2 Hz, 1H), 4.94 (t, J = 5.6 Hz, 1H), 4.89 (t, J = 5.6 Hz, 1H), 3.95 (m, 2H), 3.79 (d, J =5.6 Hz, 2H), 3.66 (m, 2H), 2.36-2.18 (m, 4H), 1.94- 1.78 (m, 2H). IC50 (uM): 0.026 |
| 904 | 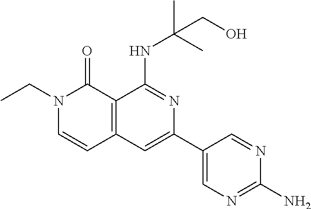 | ESI-MS m/z 355.10 (MH⁺). ¹H NMR (DMSO-d₆) δ 9.73 (s, 1H), 8.89 (s, 2H) 7.59 (d, J = 7.2 Hz, 1H), 7.05 (s, 1H),7.03 (s, 2H) 6.39 (d, J = 7.2 Hz, 1H), 5.04 (t, J = 5.6 Hz, 1H), 3.92 (q, J = 7.2 Hz, 2H), 3.65 (d, J = 5.6 Hz, 2H), 1.44 (s, 6H), 1.23 (t, J = 7.2 Hz, 3H). IC50 (uM): 0.004 |
| 905 | 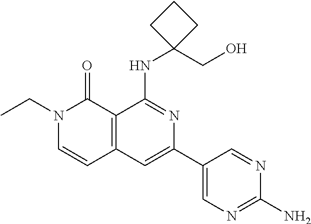 | ESI-MS m/z 367.10 (MH⁺). <IC50 (uM): 0.001009 |
| 906 | 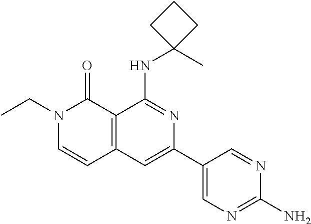 | ESI-MS m/z 351.10 (MH⁺). IC50 (uM): 0.029 |
| 907 | 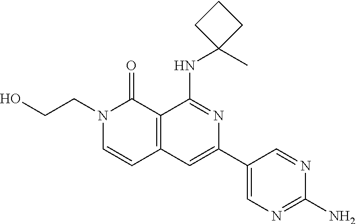 | ESI-MS m/z 367.10 (MH⁺). ¹H NMR (DMSO-d₆) δ 9.59 (s, 1H), 8.93 (s, 2H), 7.50 (d, J = 7.2 Hz, 1H), 7.08 (s, 1H), 7.03 (s, 2H), 6.37 (d, J = 7.2 Hz, 1H) 4.88 (s, 1H), 3.95 (m, 2H), 3.65 (m, 2H), 2.44-2.32 (m, 2H), 2.18- 2.08 (m, 2H), 1.96-1.82 (m, 2H), 1.62 (s, 3H). IC50 (uM): <0.000847 |
| 908 | 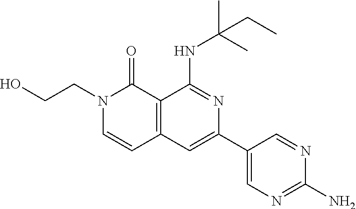 | ESI-MS m/z 369.10 (MH⁺). ¹H NMR (DMSOd₆) δ 9.63 (s, 1H), 8.91 (s, 2H), 7.49 (d, J = 7.2 Hz, 1H), 7.06 (s, 1H), 7.03 (s, 2H), 6.36 (d, J = 7.2 Hz, 1H), 4.89 (t, J = 5.6 Hz, 1H), 3.95 (m, 2H), 3.64 (m, 2H), 1.93 (q, J = 7.2 Hz, 2H), 1.47 (s, 6H), IC50 (uM): 0.85 (t, J = 7.2 Hz, 3H). IC50 (uM): 0.021 |
| 909 | 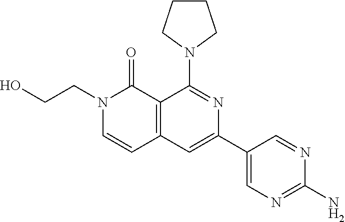 | ESI-MS m/z 353.10 (MH⁺). IC50 (uM): 0.546 |
| 910 | 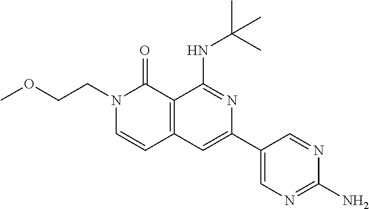 | ESI-MS m/z 369.10 (MH⁺). IC50 (uM): 0.03 |
| 911 | 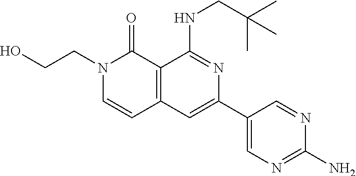 | ESI-MS m/z 369.10 (MH⁺). IC50 (uM): 0.094 |
| 912 | 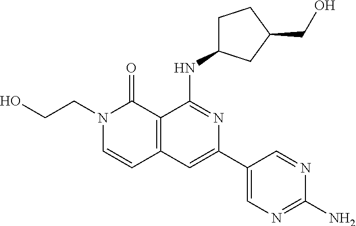 | ESI-MS m/z 397.10 (MH⁺). IC50 (uM): 0.002 |
| 913 | 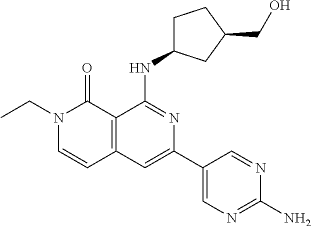 | ESI-MS m/z 381.10 (MH⁺). ¹H NMR (DMSO-d₆) δ 9.50 (d, J = 6.4 Hz, 1H), 8.94 (s, 2H), 7.60 (d, J = 7.6 Hz, 1H), 7.06 (s, 1H), 7.02 (s, 2H), 6.41 (d, J = 7.2 Hz, 1H), 4.55 (t, J = 5.2 Hz, 1H), 4.44 (m, 1H), 3.92 (m, 2H), 3.37 (m, 2H), 2.34-2.24 (m, 1H), 2.18-2.04 (m, 2H), 1.80-1.68 (m, 1H), 1.56-1.42 (m, 2H), 1.23 (t, J = 7.2 Hz, 3H), 1.20- 1.14 (m, 1H). IC50 (uM): 0.005 |
| 914 | 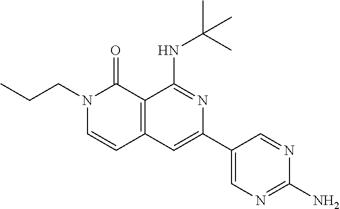 | ESI-MS m/z 353.10 (MH⁺). IC50 (uM): 0.108 |
| 915 | 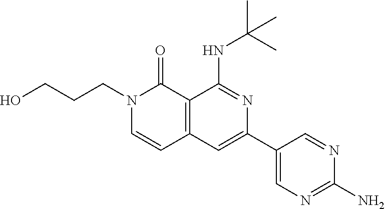 | ESI-MS m/z 369.10 (MH⁺). ¹H NMR (DMSO-d₆) δ 9.69 (s, 1H), 8.92 (s, 2H), 7.54 (d, J = 7.2 Hz, 1H), 7.07 (s, 1H), 7.04 (s, 2H), 6.39 (d, J = 7.2 Hz, 1H), 4.61 (t, J = 5.2 Hz, 1H), 3.94 (m, 2H), 3.42 (m, 2H), 1.80 (m, 2H), 1.52 (s, 9H). IC50 (uM): 0.026 |
| 916 | 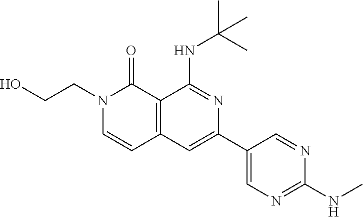 | ESI-MS m/z 368.80 (MH⁺). |
| 917 | 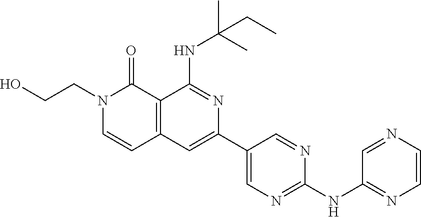 | ESI-MS m/z 369.20 (MH⁺). IC50 (uM): 0.041 |
| 918 | 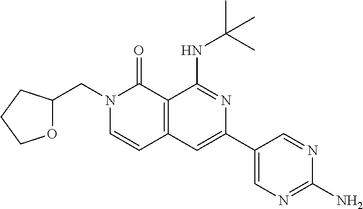 | ESI-MS m/z 395.10 (MH⁺). IC50 (uM): 0.035 |
| 919 | 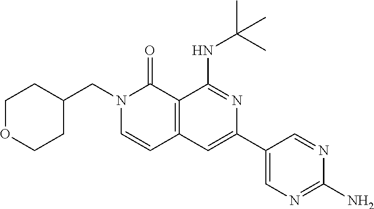 | ESI-MS m/z 409.20 (MH⁺). ¹H NMR (DMSO-d₆) δ 9.66 (s, 1H), 8.92 (s, 2H), 7.54 (d, J = 7.2 Hz, 1H), 7.07 (s, 1H), 7.04 (s, 2H), 6.37 (d, J = 7.2 Hz, 1H), 3.94 (s, 1H), 3.83 (dm, J = 7.2 Hz, 2H), 3.78 (dm, J = 7.2 Hz, 2H), 3.24 (m, 2H), 1.52 (s, 9H), 1.46 (m, 1H), 1.42 (m, 1H), 1.32-1.22 (m, 2H). IC50 (uM): 0.081 |
| 920 | 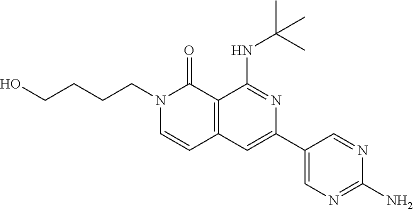 | ESI-MS m/z 383.20 (MH⁺). IC50 (uM): 0.018 |
| 921 | 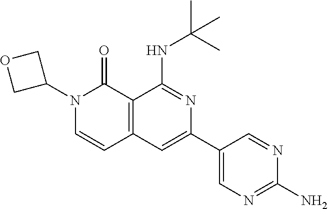 | ESI-MS m/z 367.10 (MH⁺). ¹H NMR (DMSO-d₆) δ 9.50 (s, 1H), 8.93 (s, 2H), 7.68 (d, J = 7.6 Hz, 1H), 7.12 (s, 1H), 7.06 (s, 2H), 6.49 (d, J = 7.6 Hz, 1H), 5.52 (m, 1H), 4.87 (m, 2H), 4.77 (m, 2H), 1.51 (s, 9H). IC50 (uM): 0.021 |
| 922 | 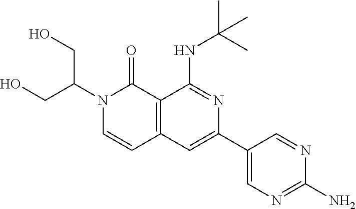 | ESI-MS m/z 385.10 (MH⁺). ¹H NMR (DMSO-d₆) δ 9.76 (s, 1H), 8.93 (s, 2H), 7.54 (d, J = 7.2 Hz, 1H), 7.06 (s, 1H), 7.03 (s, 2H), 6.38 (d, J = 7.2 Hz, 1H), 4.94- 4.88 (m, 3H), 3.76-3.64 (m, 4H), 153 (s, 9H). IC50 (uM): 0.006 |
| 923 | 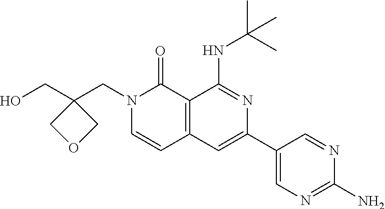 | ESI-MS m/z 411.20 (MH⁺). ¹H NMR (DMSO-d₆) δ 9.59 (s, 1H), 8.92 (s, 2H), 7.54 (d, J = 7.6 Hz, 1H), 7.09 (s, 1H), 7.05 (s, 2H), 6.41 (d, J = 7.6 Hz, 1H), 5.12 (t, J = 5.2 Hz, 1H), 4.49 (d, J = 6.4 Hz, 2H), 4.25 (d, J = 6.0 Hz, 2H), 4.16 (s, 2H), 3.60 (d, J = 5.2 Hz, 2H), 1.52 (s, 9H). IC50 (uM): 0.01 |
| 924 | 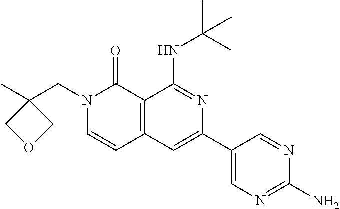 | ESI-MS m/z 395.10 (MH⁺). IC50 (uM): 0.013 |
| 925 | 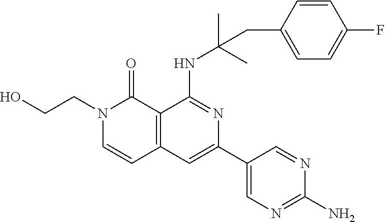 | ESI-MS m/z 449.20 (MH⁺). IC50 (uM): 0.145 |
| 926 | 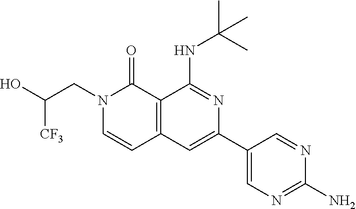 | ESI-MS m/z 423.20 (MH⁺). IC50 (uM): 0.05 |
| 927 | 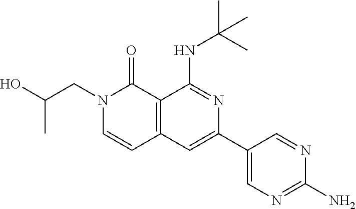 | ESI-MS m/z 369.20 (MH⁺). ¹H NMR (DMSO-d₆) δ 9.68 (s, 1H), 8.93 (s, 2H), 7.47 (d, J = 7.2 Hz, 1H), 7.06 (s, 1H), 7.03 (s, 2H), 6.35 (d, J = 7.2 Hz, 1H), 4.89 (d, J = 5.2 Hz, 1H), 4.00 (dd, J = 13.2, 4.0 Hz, 1H), 3.97-3.90 (m, 1H), 3.57 (dd, J = 13.2, 8.0 Hz, 1H), 1.52 (s, 9H), 1.09 (d, J = 6.4 Hz, 3H). IC50 (uM): 0.014 |
| 928 | 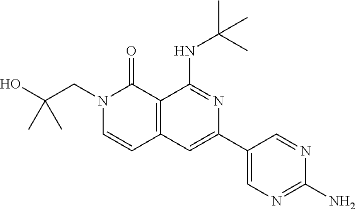 | ESI-MS m/z 383.20 (MH⁺). IC50 (uM): 0.044 |
| 929 | 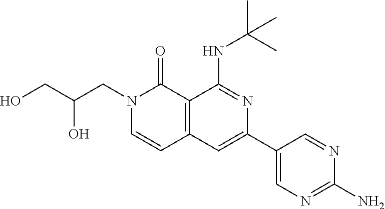 | ESI-MS m/z 385.10 (MH⁺). ¹H NMR (DMSO-d₆) δ 9.69 (s, 1H), 8.93 (s, 2H), 7.46 (d, J = 7.2 Hz, 1H), 7.06 (s, 1H), 7.03 (s, 2H), 6.36 (d, J = 7.2 Hz, 1H), 4.95 (d, J = 6.0 Hz, 1H), 4.75 (t, J = 6.0 Hz, 1H), 4.24 (dd, J = 13.2, 3.2 Hz, 1H, 3.78 (m, 1H), 3.53 (dd, J = 13.2, 8.8 Hz, 1H), 3.44-3.34 (m, 2H), 1.52 (s, 9H). IC50 (uM): 0.008 |
| 930 | 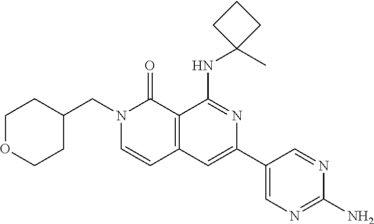 | ESI-MS m/z 421.20 (MH⁺). IC50 (uM): 0.081 |
| 931 | 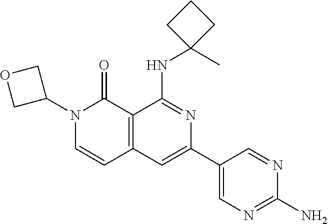 | ESI-MS m/z 379.10 (MH⁺). IC50 (uM): 0.015 |
| 932 | 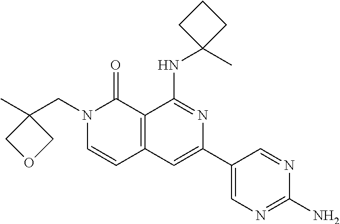 | ESI-MS m/z 407.20 (MH⁺). IC50 (uM): 0.057 |
| 933 | 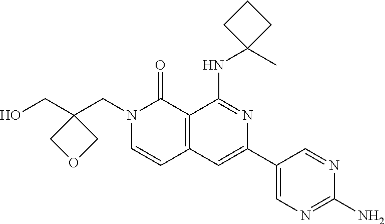 | ESI-MS m/z 423.10 (MH⁺). ¹H NMR (DMSO-d₆) δ 9.50 (s, 1H), 8.92 (s, 2H), 7.55 (d, J = 7.2 Hz, 1H), 7.10 (s, 1H), 7.04 (s, 2H), 6.41 (d, J = 7.2 Hz, 1H), 5.12 (t, J = 5.2 Hz, 1H), 4.50 (d, J = 6.4 Hz, 2H), 4.25 (d, J =6.4 Hz, 2H), 4.16 (s, 2H), 3.60 (d, J = 5.2 Hz, 2H), 2.42-2.32 (m, 2H), 2.18-2.10 (m, 2H), 1.98- 1.80 (m, 2H), 1.62 (s, 3H). IC50 (uM): 0.025 |
| 934 | 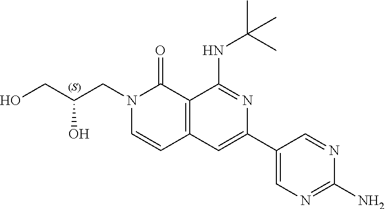 | ESI-MS m/z 385.10 (MH⁺). ¹H NMR (DMSO-d₆) δ 9.69 (s, 1H), 8.93 (s, 2H), 7.46 (d, J = 7.2 Hz, 1H), 7.06 (s, 1H), 7.03 (s, 2H), 6.36 (d, J = 7.2 Hz, 1H), 4.95 (d, J = 6.0 Hz, 1H), 4.75 (t, J = 6.0 Hz, 1H), 4.24 (dd, J = 13.2, 3.2 Hz, 1H), 3.78 (m, 1H), 3.53 (dd, J = 13.2, 8.8 Hz, 1H), 3.44-3.34 (m, 2H), 1.52 (s, 9H). IC50 (uM): 0.009 |
| 935 | 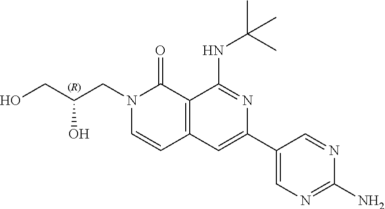 | ESI-MS m/z 385.10 (MH⁺). ¹H NMR (DMSO-d₆) δ 9.69 (s, 1H), 8.93 (s, 2H), 7.46 (d, J = 7.2 Hz, 1H), 7.06 (s, 1H), 7.03 (s, 2H), 6.36 (d, J = 7.2 Hz, 1H), 4.95 (d, J = 6.0 Hz, 1H), 4.75 (t, J = 6.0 Hz, 1H), 4.24 (dd, J = 13.2, 3.2 Hz, 1H), 3.78 (m, 1H), 3.53 (dd, J = 13.2, 8.8 Hz, 1H), 3.44- 3.34 (m, 2H), 1.52 (s, 9H). IC50 (uM): 0.008 |
| 936 | 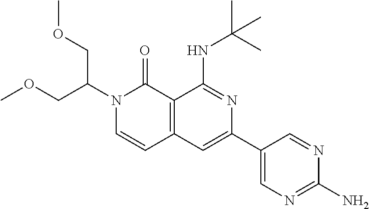 | ESI-MS m/z 413.20 (MH⁺). IC50 (uM): 0.015 |
| 937 | 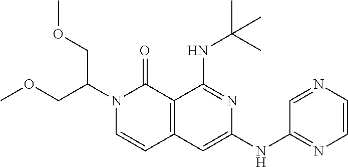 | ESI-MS m/z 413.20 (MH⁺). IC50 (uM): 0.012 |
| 938 | 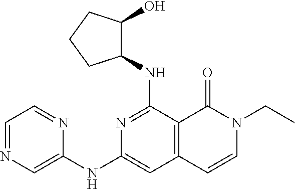 | ESI-MS m/z 367.2 (MH⁺). IC50 (uM): 0.024 |
| 939 | 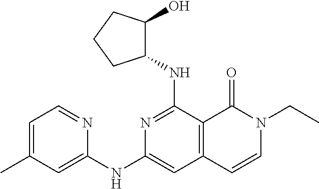 | ESI-MS m/z 380.2 (MH⁺). IC50 (uM): 1.046 |
| 940 | 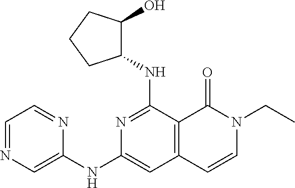 | ESI-MS m/z 367.2 (MH⁺). IC50 (uM): 0.483 |
| 941 | 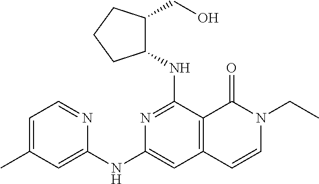 | ESI-MS m/z 394.2 (MH⁺). IC50 (uM): 0.226 |
| 942 | 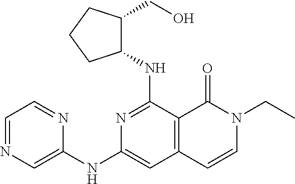 | ESI-MS m/z 381.2 (MH⁺). IC50 (uM): 0.087 |
| 943 | 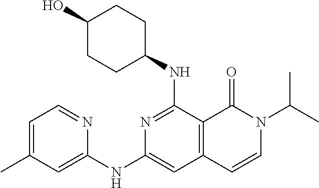 | ESI-MS m/z 408.2 (MH⁺). IC50 (uM): 0.039 |
| 944 | 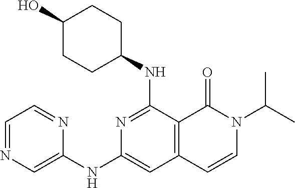 | ESI-MS m/z 395.2 (MH⁺). ¹H NMR (MeOH-d₄) δ 8.49 (d, J = 7.2 Hz, 1H), 8.35 (m, 2H), 8.81 (dd, J = 7.2 and 7.6 Hz, 1H), 6.60 (t, J = 6.0 Hz, 1H), 6.24 (s, 1H), 5.23 (m, 1H), 3.90~4.07 (m, 2H), 1.79~2.22 (m, 8H), 1.42 (d, J = 6.8 Hz, 6H). IC50 (uM): 0.032 |
| 945 | 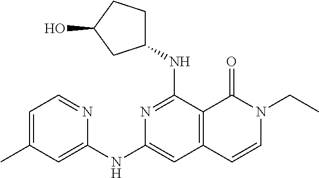 | ESI-MS m/z 38 IC50 (uM): 0.2 (MH⁺). ¹H NMR (MeOH-d₄) δ 8.35 (d, J = 6.4 Hz, 1H), 7.54 (d, J = 7.2 Hz, 1H), 7.13 (d, J = 6.4 Hz, 1H), 7.07 (s, 1H), 6.64 (d, J = 7.2 Hz, 1H), 4.51 (m, 2H), 4.00 (q, J = 7.2 Hz, 2H), 2.51 (s, 3H), 2.45 (m, 1H), 2.34 (m, 1H), 2.20 (m, 1H), 1.80 (m, 2H), 1.33 (t, J = 7.2 Hz, 3H). IC50 (uM): 0.023 |
| 946 | 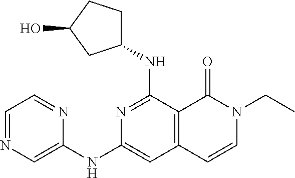 | ESI-MS m/z 367.2 (MH⁺). IC50 (uM): 0.017 |
| 947 | 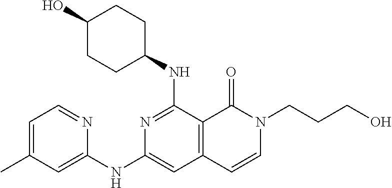 | ESI-MS m/z 424.2 (MH⁺). IC50 (uM): 0.019 |
| 948 | 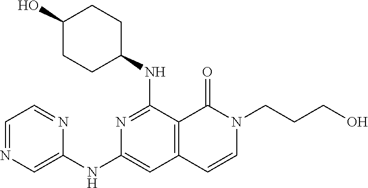 | ESI-MS m/z 411.2 (MH⁺). ¹H NMR (MeOH-d₄) δ 8.47 (s, 1H), 8.34 (m, 2H), 7.72 (d, J = 7.6 Hz, 1H), 6.64 (d, J = 7.2 Hz, 1H), 6.32 (s, 1H), 4.12 (t, J = 6.8 Hz, 2H), 3.94 (m, 2H), 3.63 (t, J = 6.0 Hz, 2H), 3.11 (m, 2H), 1.79~2.06 (m, 10H). IC50 (uM): 0.018 |
| 949 | 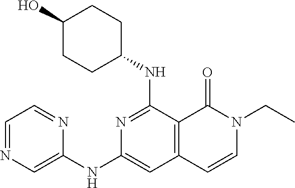 | ESI-MS m/z 381.2 (MH⁺). IC50 (uM): 0.05 |
| 950 | 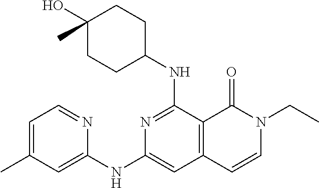 | ESI-MS m/z 408.2 (MH⁺). ¹H NMR (MeOH-d₄) δ 8.15 (d, J = 5.6 Hz, 1H), 7.57 (d, J = 7.6 Hz, 1H), 7.12 (dd, J = 1.2 and 6.0 HZ, 1H), 7.04 (s, 1H), 6.44 (d, J = 7.6 Hz, 2H), 6.16 (s, 1H), 4.01 (q, J = 7.2 Hz, 2H), 3.85 (m, 1H), 2.49 (s, 3H), 1.60~2.09 (m, 8H), 1.34 (t, J = 6.8 Hz, 3H), 1.32 (s, 3H). IC50 (uM): 0.053 |
| 951 | 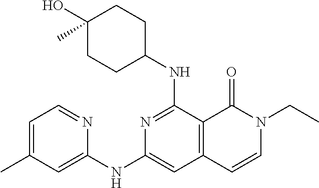 | ESI-MS m/z 408.2 (MH⁺). IC50 (uM): 0.092 |
| 952 | 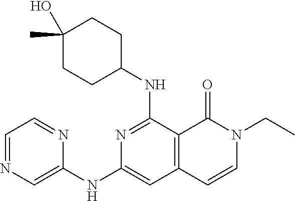 | ESI-MS m/z 395.2 (MH⁺). IC50 (uM): 0.051 |
| 953 | 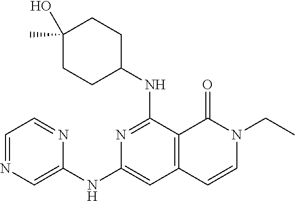 | ESI-MS m/z 367.2 (MH⁺). IC50 (uM): 0.043 |
| 954 | 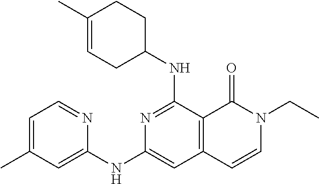 | ESI-MS m/z 390.2 (MH⁺). IC50 (uM): 0.754 |
| 955 | 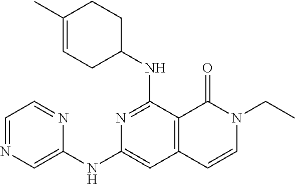 | ESI-MS m/z 377.2 (MH⁺). IC50 (uM): 0.376 |
| 956 | 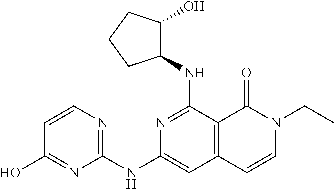 | ESI-MS m/z 383.2 (MH⁺). IC50 (uM): 0.066 |
| 957 | 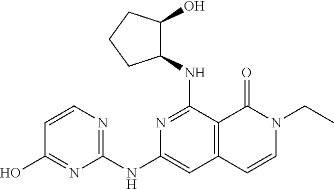 | ESI-MS m/z 383.2 (MH⁺). ¹H NMR (MeOH-d₄) δ 8.12 (d, J = 6.8 Hz, 1H), 7.62 (d, J = 7.6 Hz, 1H), 6.44 (d, J = 7.6 Hz, 1H), 6.39 (d, J = 6.4 Hz, 1H), 6.27 (s, 1H), 4.41 (q, J = 8.8 Hz, 1H), 4.13 (m, 1H), 4.01 (q, J = 7.2 Hz, 2H), 2.35 (m, 1H), 2.11 (m, 1H), 1.97 (m, 1H), 1.80 (m, 3H), 1.34 (t, J = 6.8 Hz, 3H). IC50 (uM): 0.015 |
| 958 | 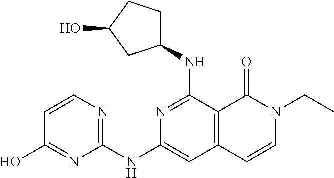 | ESI-MS m/z 383.2 (MH⁺). IC50 (uM): 0.022 |
| 959 | 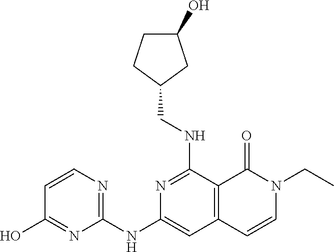 | ESI-MS m/z 397.2 (MH⁺). IC50 (uM): 0.041 |
| 960 | 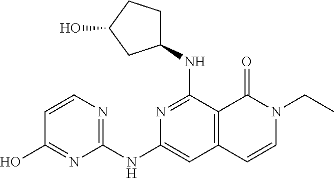 | ESI-MS m/z 383.2 (MH⁺). IC50 (uM): 0.061 |
| 961 | 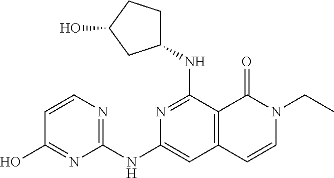 | ESI-MS m/z 383.2 (MH⁺). IC50 (uM): 0.023 |
| 962 | 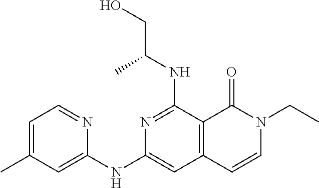 | ESI-MS m/z 354.2 (MH⁺). IC50 (uM): 0.092 |
| 963 | 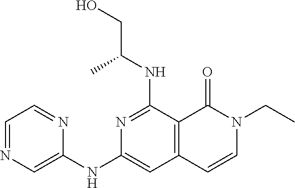 | ESI-MS m/z 341.2 (MH⁺). IC50 (uM): 0.147 |
| 964 | 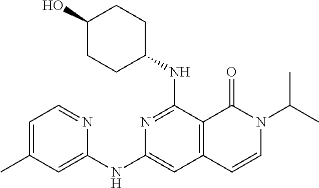 | ESI-MS m/z 408.2 (MH⁺). IC50 (uM): 0.051 |
| 965 | 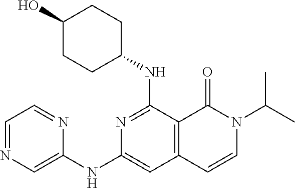 | ESI-MS m/z 395.2 (MH⁺). IC50 (uM): 0.061 |
| 966 | 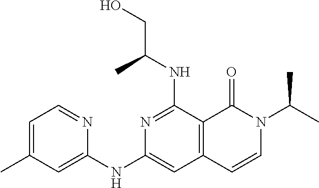 | ESI-MS m/z 368.2 (MH⁺). IC50 (uM): 0.029 |
| 967 | 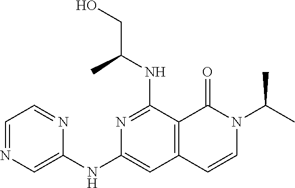 | ESI-MS m/z 355.2 (MH⁺). ¹H NMR (DMSO-d₆) δ 9.83 (s, 1H), 9.68 (d, J = 8.0 Hz, 1H), 9.18 (s, 1H), 8.26 (dd, J = 1.6 and 2.4 Hz, 1H), 8.10 (d, J = 2.8 Hz, 1H), 7.49 (d, J = 7.6 Hz, 1H), 6.67 (s, 1H), 6.32 (d, J = 7.2 Hz, 1H), 5.08 (m, 1H), 4.88 (t, J = 4.8 Hz, 1H), 4.25 (m, 1H), 3.53 (t, J = 4.8 Hz, 2H), 1.28 (d, J = 6.8 Hz, 6H), 1.22 (d, J = 6.4 Hz, 3H). IC50 (uM): 0.017 |
| 968 | 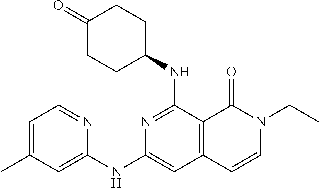 | ESI-MS m/z 393.2 (MH⁺). IC50 (uM): 0.059 |
| 969 | 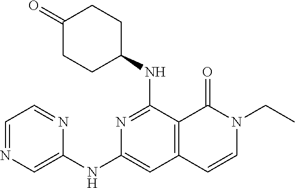 | ESI-MS m/z 38 IC50 (uM): 0.2 (MH⁺). IC50 (uM): 0.081 |
| 970 | 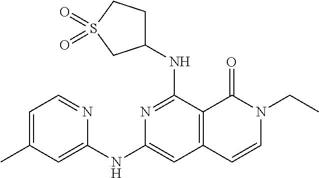 | ESI-MS m/z 414.2 (MH⁺). IC50 (uM): 0.075 |
| 971 | 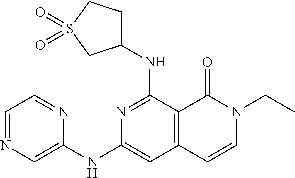 | ESI-MS m/z 401.2 (MH⁺). IC50 (uM): 0.145 |
| 972 | 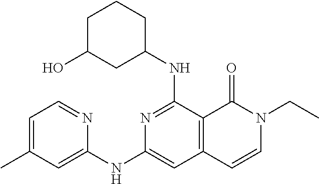 | ESI-MS m/z 394.2 (MH⁺). IC50 (uM): 0.059 |
| 973 | 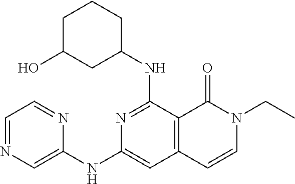 | ESI-MS m/z 381.2 (MH⁺). IC50 (uM): 0.026 |
| 974 | 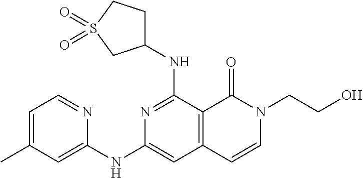 | ESI-MS m/z 430.2 (MH⁺). IC50 (uM): 0.203 |
| 975 | 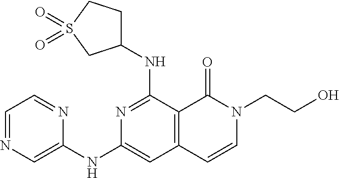 | ESI-MS m/z 417.2 (MH⁺). IC50 (uM): 0.37 |
| 976 | 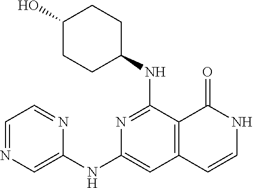 | ESI-MS m/z 353.2 (MH⁺). IC50 (uM): 0.005 |
| 977 | 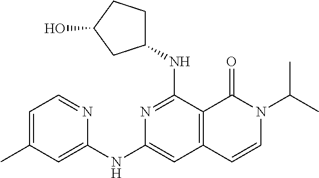 | ESI-MS m/z 394.2 (MH⁺). IC50 (uM): 0.02 |
| 978 | 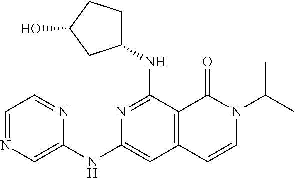 | ESI-MS m/z 381.2 (MH⁺). IC50 (uM): 0.016 |
| 979 | 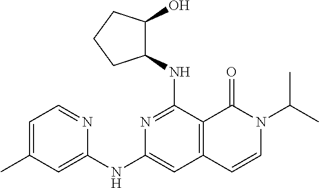 | ESI-MS m/z 394.2 (MH⁺). |
| 980 | 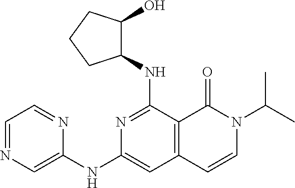 | ESI-MS m/z 381.2 (MH⁺). IC50 (uM): 0.036 |
| 981 | 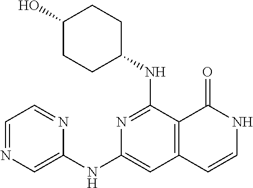 | ESI-MS m/z 353.2 (MH⁺). IC50 (uM): 0.015 |
| 982 | 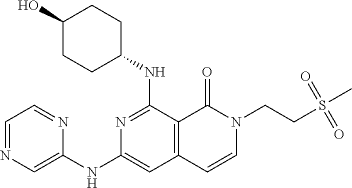 | ESI-MS m/z 459.2 (MH⁺). IC50 (uM): 0.022 |
| 983 | 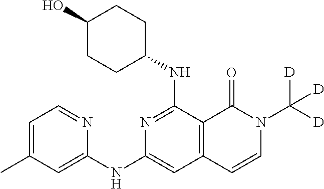 | ESI-MS m/z 383.2 (MH⁺). ¹H NMR (MeOH-d₄) δ 8.17 (d, J = 6.0 Hz, 1H), 7.54 (d, J = 7.6 Hz, 1H), 7.12 (d, J = 6.0 Hz, 1H), 7.06 (s, 1H) 7.63 (d, J = 7.6 Hz, 1H), 6.18 (s, 1H), 4.04 (bs, 1H), 3.86 (m, 1H), 2.50 (s, 3H), 1.85~2.09 (m, 6H), 1.76 (m, 1H). IC50 (uM): 0.051 |
| 984 | 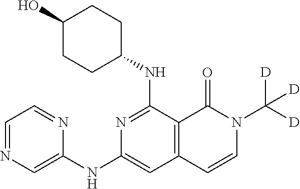 | ESI-MS m/z 37 IC50 (uM): 0.2 (MH⁺). IC50 (uM): 0.029 |
| 985 | 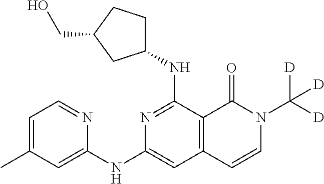 | ESI-MS m/z 383.2 (MH⁺). IC50 (uM): 0.041 |
| 986 | 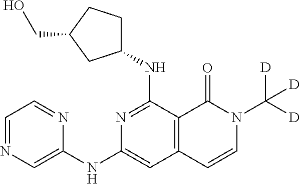 | ESI-MS m/z 370.2 (MH⁺). IC50 (uM): 0.009 |
| 987 | 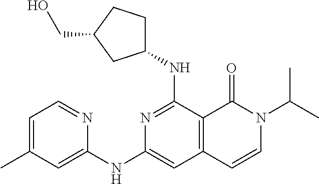 | ESI-MS m/z 408.2 (MH⁺). IC50 (uM): 0.06 |
| 988 | 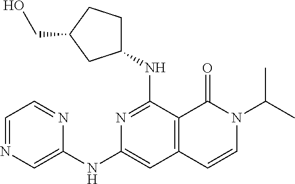 | ESI-MS m/z 395.2 (MH⁺). IC50 (uM): 0.003 |
| 989 | 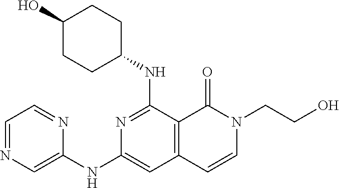 | ESI-MS m/z 397.2 (MH⁺). IC50 (uM): 0.015 |
| 990 | 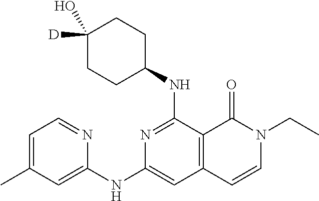 | ESI-MS m/z 395.2 (MH⁺). IC50 (uM): 0.02 |
| 991 | 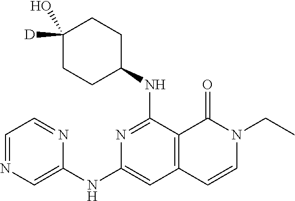 | ESI-MS m/z 382.2 (MH⁺). IC50 (uM): 0.029 |
| 992 | 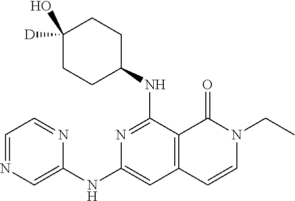 | ESI-MS m/z 382.2 (MH⁺). IC50 (uM): 0.012 |
| 993 | 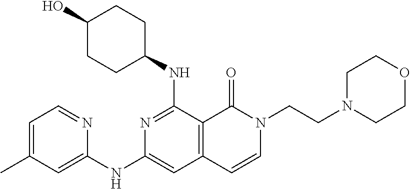 | ESI-MS m/z 479.2 (MH⁺). IC50 (uM): 0.067 |
| 994 | 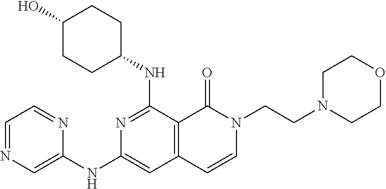 | ESI-MS m/z 466.2 (MH⁺). IC50 (uM): 0.108 |
| 995 | 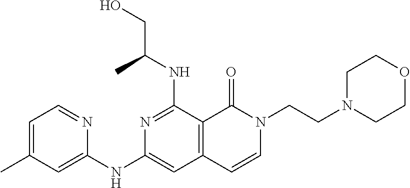 | ESI-MS m/z 439.2 (MH⁺). IC50 (uM): 0.053 |
| 996 | 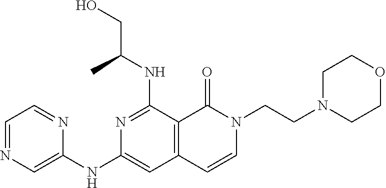 | ESI-MS m/z 426.2 (MH⁺). IC50 (uM): 0.158 |
| 997 | 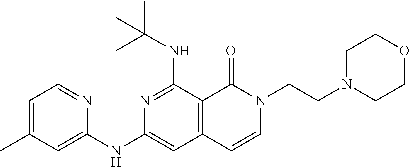 | ESI-MS m/z 437.2 (MH⁺). IC50 (uM): 0.296 |
| 998 | 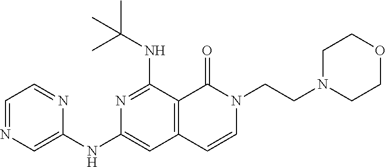 | ESI-MS m/z 424.2 (MH⁺). IC50 (uM): 0.205 |
| 999 | 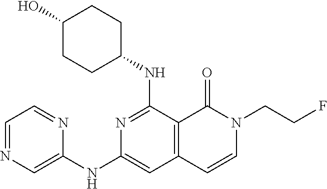 | ESI-MS m/z 399.2 (MH⁺). IC50 (uM): 0.01 |
| 1000 | 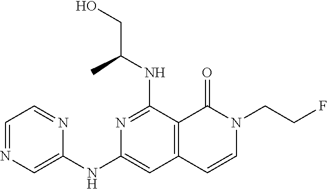 | ESI-MS m/z 359.2 (MH⁺). IC50 (uM): 0.074 |
| 1001 | 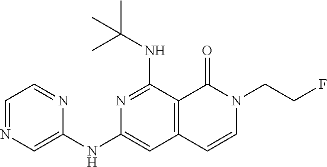 | ESI-MS m/z 357.2 (MH⁺). ¹H NMR (MeOH-d₄) δ 8.51 (s, 1H), 8.37 (d, J = 3.2 Hz, 1H), 8.34 (dd, J = 1.2 and 2.8 Hz, 1H), 8.02 (dd, J = 1.2 and 8.0 Hz, IC50 (uM): 0.5H), 7.67 (d, J = 7.2 Hz, 1H), 7.47 (t, J = 7.6 Hz, IC50 (uM): 0.5H), 6.52 (d, J = 7.6 Hz, 1H), 4.79 (t, J = 4.8 Hz, 1H), 4.67 (t, J = 4.8 Hz, 1H), 4.36 (t, J = 4.8 Hz, 1H), 4.29 (t, J = 4.8 Hz, 1H), 1.71 (s, 9H). IC50 (uM): 0.049 |
| 1002 | 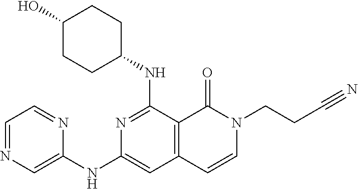 | ESI-MS m/z 406.2 (MH⁺). IC50 (uM): 0.014 |
| 1003 | 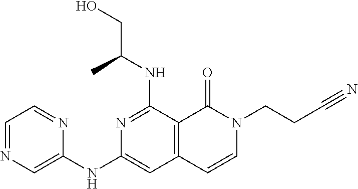 | ESI-MS m/z 366.2 (MH⁺). IC50 (uM): 0.027 |
| 1004 | 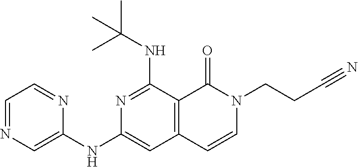 | ESI-MS m/z 364.2 (MH⁺). ¹H NMR (DMSO-d₆) δ 9.75 (s, 1H), 9.66 (s, 1H), 8.88 (s, 1H), 8.26 (dd, J = 1.6 and 2.8 Hz, 1H), 8.09 (d, J = 2.8 Hz, 1H), 7.46 (d, J = 7.2 Hz, 1H), 6.97 (s, 1H), 6.33 (d, J = 7.6 Hz, 1H), 4.10 (t, J = 6.8 Hz, 2H), 2.96 (t, J = 6.4 Hz, 2H_, 1.50 (s, 9H). IC50 (uM): 0.019 |
| 1005 | 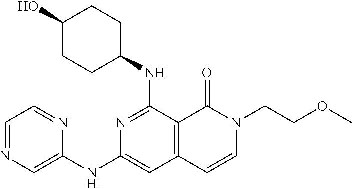 | ESI-MS m/z 411.2 (MH⁺). IC50 (uM): 0.017 |
| 1006 | 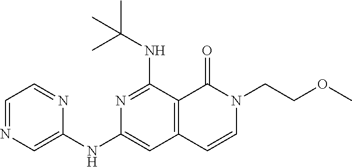 | ESI-MS m/z 369.2 (MH⁺). IC50 (uM): 0.096 |
| 1007 | 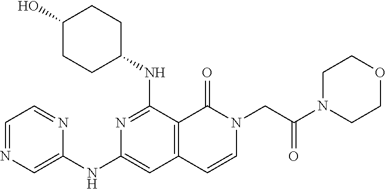 | ESI-MS m/z 48 IC50 (uM): 0.2 (MH⁺). IC50 (uM): 0.132 |
| 1008 | 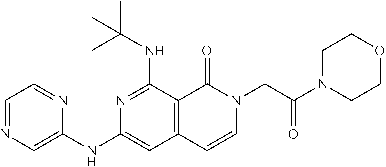 | ESI-MS m/z 438.2 (MH⁺). IC50 (uM): 0.238 |
| 1009 | 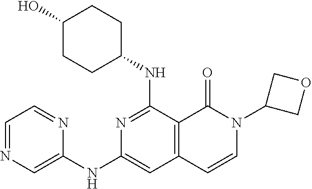 | ESI-MS m/z 409.2 (MH⁺). IC50 (uM): 0.019 |
| 1010 | 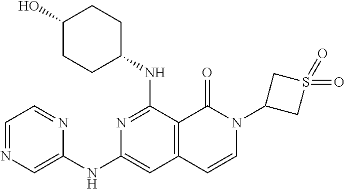 | ESI-MS m/z 457.2 (MH⁺). IC50 (uM): 0.01 |
| 1011 | 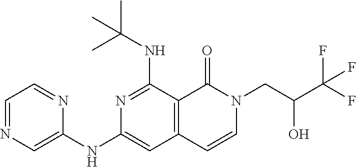 | ESI-MS m/z 423.2 (MH⁺). IC50 (uM): 0.459 |
| 1012 | 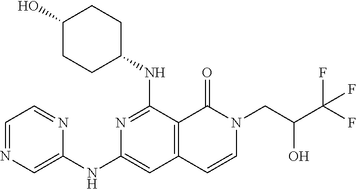 | ESI-MS 465.2 (MH⁺). ¹H NMR (MeOH-d₄) δ 9.57 (d, J = 7.6 Hz, 1H), 9.26 (s, 1H), 8.16 (dd, J = 1.2 and 2.4 Hz, 1H), 7.95 (d, J = 2.8 Hz, 1H), 7.16 (d, J = 7.6 Hz, 1H), 6.38 (s, 1H), 6.17 (d, J = 7.6 Hz, 1H), 4.42 (dd, J = 3.2 and 13.6 Hz, 1H), 4.28 (m, 1H), 4.12 (bs, 1H), 3.73 (bs, 1H), 3.56 (dd, J = 9.6 and 13.6 Hz, 1H), 1.61~1.82 (m, 8H). IC50 (uM): 0.056 |
| 1013 | 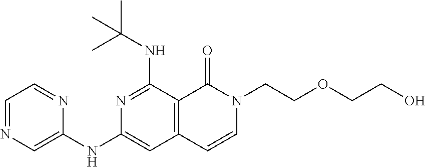 | ESI-MS m/z 399.2 (MH⁺). IC50 (uM): 0.069 |
| 1014 | 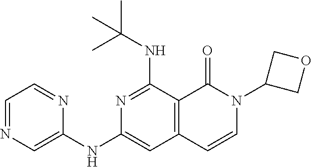 | ESI-MS m/z 367.2 (MH⁺). IC50 (uM): 0.12 |
| 1015 | 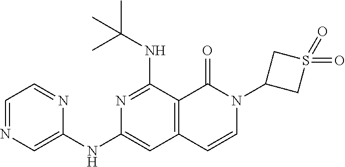 | ESI-MS m/z 415.2 (MH⁺). IC50 (uM): 0.138 |
| 1016 | 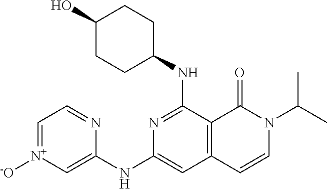 | ESI-MS m/z 411.2 (MH⁺). IC50 (uM): 0.105 |
| 1017 | 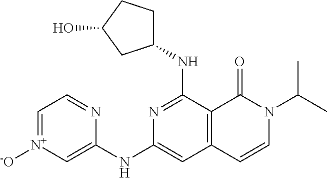 | ESI-MS m/z 397.2 (MH⁺). IC50 (uM): 0.025 |
| 1018 | 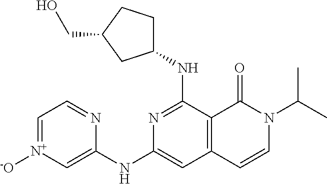 | ESI-MS m/z 411.2 (MH⁺). ¹H NMR (MeOH-d₄) δ 9.47 (s, 1H), 8.11 (d, J = 4.0 Hz, 1H), 7.74 (dd, J = 1.6 and 4.0 Hz, 1H), 7.27 (d, J = 7.6 Hz, 1H), 6.23 (d, J = 7.2 Hz, 1H), 6.12 (s, 1H), 5.06 (m, 1H), 4.35 (m, 1H), 3.45 (d, J = 6.8 Hz, 2H), 2.33 (m, 1H), 2.08~2.23 (m, 2H), 1.83 (m, 1H), 1.41~1.59 (m, 2H), 1.25 (d, J = 6.8 Hz, 6H). IC50 (uM): 0.013 |
| 1019 | 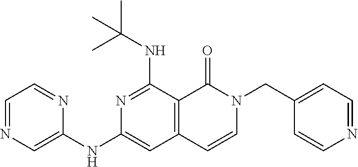 | ESI-MS m/z 402.2 (MH⁺). IC50 (uM): 0.301 |
| 1020 | 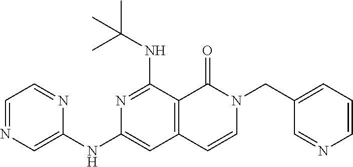 | ESI-MS m/z 402.2 (MH⁺). IC50 (uM): 0.164 |
| 1021 | 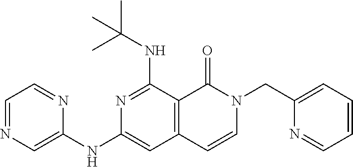 | ESI-MS m/z 402.2 (MH⁺). IC50 (uM): 0.219 |
| 1022 | 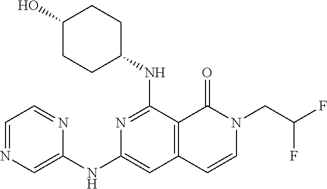 | ESI-MS m/z 417.2 (MH⁺). ¹H NMR (MeOH-d₄) δ 8.62 (s, 1H), 8.33 (s, 2H), 8.02 (d, J = 6.8 Hz, 1H), 7.60 (m, 1H), 7.47 (t, J = 7.2 Hz, 1H), 6.52 (d, J = 7.6 Hz, 1H), 6.37 (t, J = 4.0 Hz, IC50 (uM): 0.25H), 6.23 (t, J = 4.0 Hz, IC50 (uM): 0.5H), 6.09 (t, J = 4.0 Hz, IC50 (uM): 0.25H), 4.40 (dt, J = 4.0 and 14.0 Hz, 2H), 3.99 (bs, 1H), 3.91 (m, 1H), 1.72~2.10 (m, 8H). IC50 (uM): 0.011 |
| 1023 | 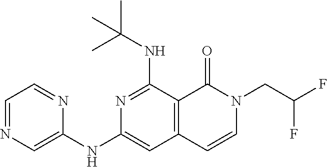 | ESI-MS m/z 375.2 (MH⁺). IC50 (uM): 0.179 |
| 1024 | 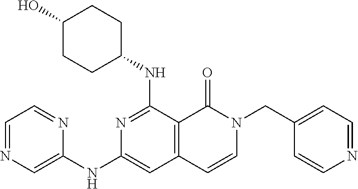 | ESI-MS m/z 444.2 (MH⁺). IC50 (uM): 0.174 |
| 1025 | 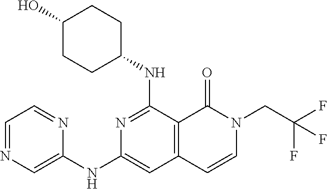 | ESI-MS m/z 435.2 (MH⁺). IC50 (uM): 0.016 |
| 1026 | 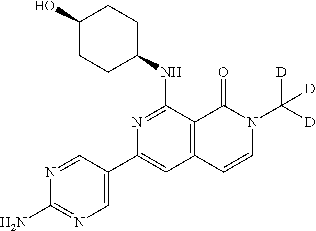 | ESI-MS m/z 370.2 (MH⁺). IC50 (uM): 0.021 |
| 1027 | 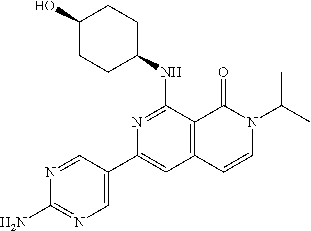 | ESI-MS m/z 395.2 (MH⁺). IC50 (uM): 0.032 |
| 1028 | 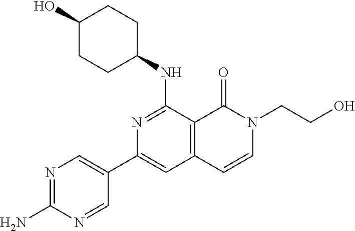 | ESI-MS m/z 397.2 (MH⁺). IC50 (uM): 0.014 |
| 1029 | 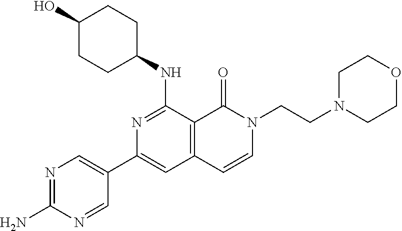 | ESI-MS m/z 466.2 (MH⁺). IC50 (uM): 0.072 |
| 1030 | 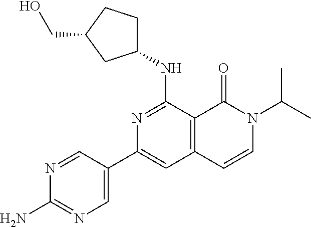 | ESI-MS m/z 395.2 (MH⁺). IC50 (uM): 0.048 |
| 1031 | 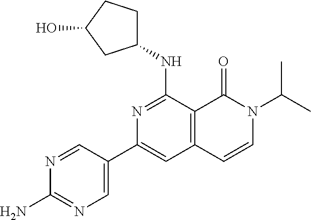 | ESI-MS m/z 381.2 (MH⁺). IC50 (uM): 0.007 |
| 1032 | 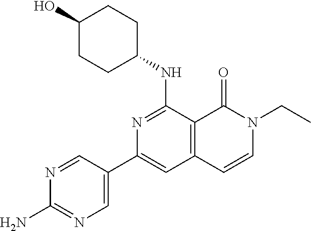 | ESI-MS m/z 381.2 (MH⁺). IC50 (uM): 0.046 |
| 1033 | 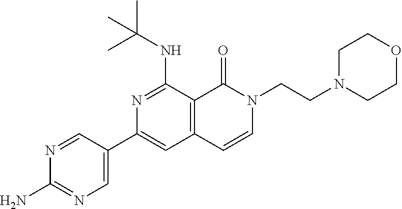 | ESI-MS m/z 424.2 (MH⁺). IC50 (uM): 0.06 |
| 1034 | 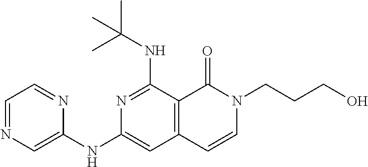 | ESI-MS m/z 369.2 (MH⁺). IC50 (uM): 0.012 |
| 1035 | 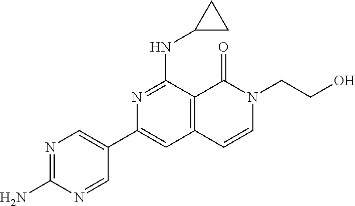 | ESI-MS m/z 339.2 (MH⁺). IC50 (uM): 0.038 |
| 1036 | 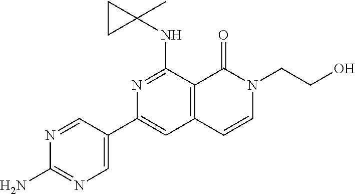 | ESI-MS m/z 353.2 (MH⁺). IC50 (uM): 0.02 |
| 1037 | 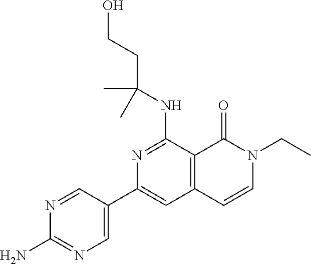 | ESI-MS m/z 369.2 (MH⁺). IC50 (uM): 0.014 |
| 1038 | 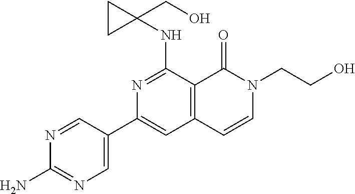 | ESI-MS m/z 353.2 (MH⁺). IC50 (uM): 0.024 |
| 1039 | 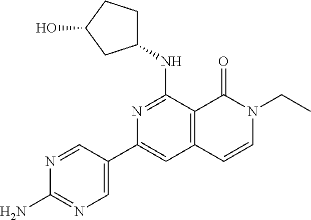 | ESI-MS m/z 367.2 (MH⁺). IC50 (uM): 0.012 |
| 1040 | 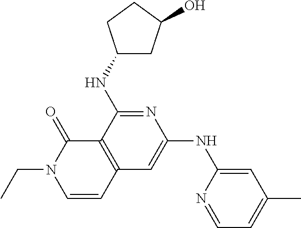 | ESI-MS m/z 380.1 (MH⁺). IC50 (uM): 0.084 |
| 1041 | 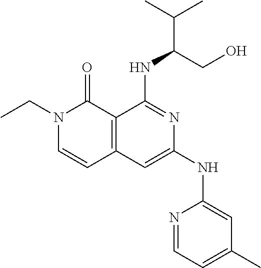 | ESI-MS m/z 382.2 (MH⁺). IC50 (uM): 0.158 |
| 1042 | 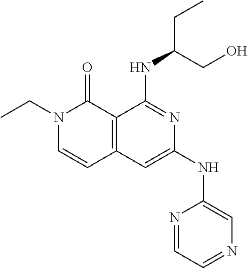 | ESI-MS m/z 355.2 (MH⁺). IC50 (uM): 0.111 |
| 1043 | 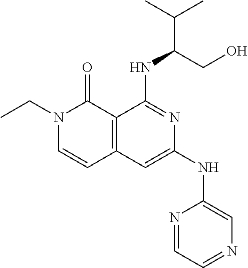 | ESI-MS m/z 369.2 (MH⁺). IC50 (uM): 0.122 |
| 1044 | 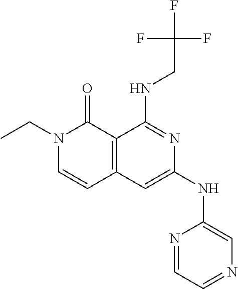 | ESI-MS m/z 365.1 (MH⁺). IC50 (uM): 0.11 |
| 1045 | 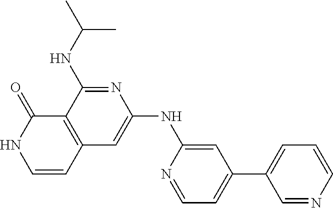 | ESI-MS m/z 373.2 (MH⁺). IC50 (uM): 0.247 |
| 1046 | 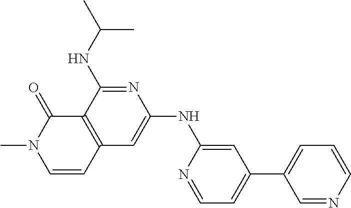 | ESI-MS m/z 387.1 (MH⁺). IC50 (uM): 0.113 |
| 1047 | 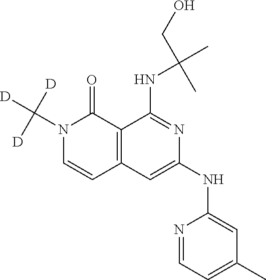 | ESI-MS m/z 357.3 (MH⁺). IC50 (uM): 0.0772 |
| 1048 | 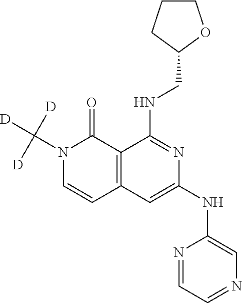 | ESI-MS m/z 356.2 (MH⁺). IC50 (uM): 0.164 |
| 1049 | 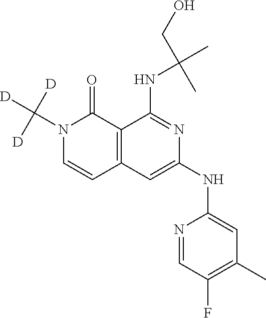 | ESI-MS m/z 375.2 (MH⁺). IC50 (uM): 0.0899 |
| 1050 | 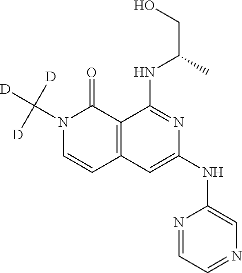 | ESI-MS m/z 330.2 (MH⁺). IC50 (uM): 0.0457 |
| 1051 | 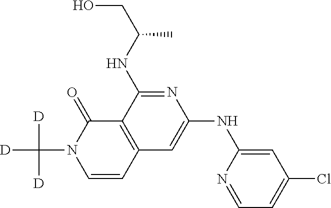 | ESI-MS m/z 363.2 (MH⁺). IC50 (uM): 0.0604 |
| 1052 | 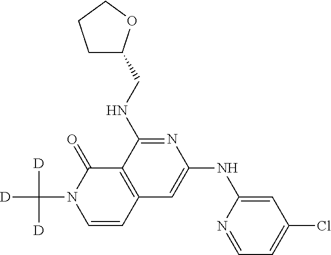 | ESI-MS m/z 389.2 (MH⁺). IC50 (uM): 0.236 |

### Assays

Compounds of the examples and Table 1 provided herein were assayed to measure their capacity to inhibit Syk kinase.

Compounds of the examples and Table 1 provided herein were assessed for their ability to inhibit Syk kinase by utilizing Caliper Life Sciences' proprietary LabChip™ technology. The off-chip incubation mobility-shift kinase assay uses a microfluidic chip to measure the conversion of a fluorescent peptide substrate to a phosphorylated product. The reaction mixture, from a microtiter plate well, is introduced through a capillary sipper onto the chip, where the nonphosphorylated substrate and phosphorylated product are separated by electrophoresis and detected via laser induced fluorescence. The signature of the fluorescence signal over time reveals the extent of the reaction. The phosphorylated product migrates through the chip faster than the non-phosphorylated substrate, and signals from the two forms of the peptide appear as distinct peaks. Caliper's data analysis software (HTSWA) determines peak heights, from which the ratio of product to the peak sum P/(P+S) and percent (%) conversion is calculated. This value is used to compare compound wells to control wells present on the plate, and thereby determine the % inhibition values for the compound. The formula used to calculate % inhibition is as follows, where C_{100%} is the average % conversion of the 100% activity wells and Cₒ% is the average % conversion of the 0% activity wells: (1−(% conversionofsample−C_{0%})/(C_{100%}−C_{0%}))*100.

Compounds (10 mM stocks in 100% DMSO) are diluted to a final concentration of 5 μM for single point inhibition experiments, and a series dilution of 10, 3, 1, 0.3, 0.1, 0.03, 0.01, 0.003, 0.001, 0.0003, 0.0001, and 0.00003 μM were made for IC₅₀ determination. Generally, 12 μL of enzyme buffer containing purified kinase (various amount; various suppliers), 100 mM HEPES, pH 7.5, 1 mM DTT (Calbiochem, 2333153), 10 mM MgCl₂ (Sigma, M-1028) or 10 mM MnCl₂ (Sigma, M-1787) (assay specific), and 0.002% Brij-35 (Sigma, B4184) are added to each well. Compound and enzyme are allowed to pre-incubate for 15 minutes. 12 μL of peptide/ATP buffer containing 100 mM HEPES, pH 7.5, 1.5 μM fluorescein-labeled peptide (specific to kinase of interest), ATP (at K_{M} apparent, Sigma, A9187), and 0.002% Brij-35 is then added to each well to initiate the reaction. The final concentration of DMSO in the well is 4%. Generally, reactions are incubated for 1 to 1.5 hours at room temperature to obtain adequate conversion of peptide to phosphorylated product in the linear range of the reaction. Reactions are terminated with the addition of 45 μL of Stop Buffer (containing 20 mM EDTA). Plates are then read on the LabChip 3000 using a 12-sipper LabChip. % conversion values and % inhibition values are obtained as provided and IC₅₀ curves of compounds are generated using GraphPad Prism Version 4 or 5.01, or XLfit Version 4.3.2. When using GraphPad Prism, a nonlinear curve fit using the sigmoidal dose response—variable slope fit was used to graph IC₅₀ curves and determine IC₅₀ values and hillslopes. When using XLfit, Fit Model 205 (4-Parameter Logistic Model) is used to generate and fit the IC₅₀ curve.

In certain embodiments, compounds of Formula (I) given in the examples and in Table 1, in free form or in pharmaceutically acceptable salt form, exhibit valuable pharmacological properties, for example, as indicated by the in vitro tests provided in this application. In general, compounds provided herein have IC₅₀ values for Syk kinase inhibition from 1 nM to 8 μM. In some examples, compounds provided herein have IC₅₀ values for Syk kinase inhibition from 1 nM to 5 μM. In some examples, compounds provided herein have IC₅₀ values for Syk kinase inhibition from 1 nM to 3 μM. In some examples, compounds provided herein have IC₅₀ values for Syk kinase inhibition from 1 nM to 2 μM. In some examples, compounds provided herein have IC₅₀ values for Syk kinase inhibition from 1 nM to 1 μM. In some examples, compounds provided herein have IC₅₀ values for Syk kinase inhibition from 1 nM to 500 nM. In some examples, compounds provided herein have IC₅₀ values for Syk kinase inhibition from 1 nM to 400 nM. In some examples, compounds provided herein have IC₅₀ values for Syk kinase inhibition from 1 nM to 300 nM. In some examples, compounds provided herein have IC₅₀ values for Syk kinase inhibition from 1 nM to 200 nM. In some examples, compounds provided herein have IC₅₀ values for Syk kinase inhibition from 1 nM to 100 nM. In some examples, compounds provided herein have IC₅₀ values for Syk kinase inhibition from 1 nM to 50 nM. In some examples, compounds provided herein have IC₅₀ values for Syk kinase inhibition from 1 nM to 25 nM. In some examples, compounds provided herein have IC₅₀ values for Syk kinase inhibition from 1 nM to 10 nM. In certain embodiments, compounds of Formula (I) exhibit a percentage inhibition of greater than 50%, or in other embodiments compounds of Formula (I) exhibit a percentage inhibition greater than about 70%, against Syk kinase.

By way of example only, the IC₅₀ for Syk inhibition by certain compounds of Formula (I) are listed in Table 1

In addition compounds of the examples and Table 1 provided herein were assayed to measure their capacity to inhibit ZAP70, KDR, FMS, FLT3, c-Kit, RET, TrkA, TrkB, TrkC, IGR-1R, Alk and c-FMS kinases.

Compounds of the examples and Table 1 provided herein were assessed for their ability to inhibit ZAP70, KDR, FMS, FLT3, c-Kit, RET, TrkA, TrkB, TrkC, IGR-1R, Alk and c-FMS kinases by utilizing Caliper Life Sciences' proprietary LabChip™ technology. The off-chip incubation mobility-shift kinase assay uses a microfluidic chip to measure the conversion of a fluorescent peptide substrate to a phosphorylated product. The reaction mixture, from a microtiter plate well, is introduced through a capillary sipper onto the chip, where the nonphosphorylated substrate and phosphorylated product are separated by electrophoresis and detected via laser induced fluorescence. The signature of the fluorescence signal over time reveals the extent of the reaction. The phosphorylated product migrates through the chip faster than the non-phosphorylated substrate, and signals from the two forms of the peptide appear as distinct peaks. Caliper's data analysis software (HTSWA) determines peak heights, from which the ratio of product to the peak sum P/(P+S) and percent (%) conversion is calculated. This value is used to compare compound wells to control wells present on the plate, and thereby determine the % inhibition values for the compound. The formula used to calculate % inhibition is as follows, where C_{100%} is the average % conversion of the 100% activity wells and C_{0%} is the average % conversion of the 0% activity wells: (1−(% conversionofsample−C_{0%})/(C_{100%}−C_{0%}))*100.

Compounds (10 mM stocks in 100% DMSO) are diluted to a final concentration of 5 μM for single point inhibition experiments, and a series dilution of 10, 3, 1, 0.3, 0.1, 0.03, 0.01, 0.003, 0.001, 0.0003, 0.0001, 0.00003 μM were made for IC₅₀ determination. Generally, 12 μL of enzyme buffer containing purified kinase (various amount; various suppliers), 100 mM HEPES, pH 7.5, 1 mM DTT (Calbiochem, 2333153), 10 mM MgCl₂ (Sigma, M-1028) or 10 mM MnCl₂ (Sigma, M-1787) (assay specific), and 0.002% Brij-35 (Sigma, B4184) are added to each well. Compound and enzyme are allowed to pre-incubate for 15 minutes. 12 μL of peptide/ATP buffer containing 100 mM HEPES, pH 7.5, 1.5 μM fluorescein-labeled peptide (specific to kinase of interest), ATP (at K_{M} apparent, Sigma, A9187), and 0.002% Brij-35 is then added to each well to initiate the reaction. The final concentration of DMSO in the well is 4%. Generally, reactions are incubated for 1 to 1.5 hours at room temperature to obtain adequate conversion of peptide to phosphorylated product in the linear range of the reaction. Reactions are terminated with the addition of 45 μL of Stop Buffer (containing 20 mM EDTA). Plates are then read on the LabChip 3000 using a 12-sipper LabChip. % conversion values and % inhibition values are obtained as provided and IC₅₀ curves of compounds are generated using GraphPad Prism Version 4 or 5.01, or XLfit Version 4.3.2. When using GraphPad Prism, a nonlinear curve fit using the sigmoidal dose response—variable slope fit was used to graph IC₅₀ curves and determine IC₅₀ values and hillslopes. When using XLfit, Fit Model 205 (4-Parameter Logistic Model) is used to generate and fit the IC₅₀ curve.

In certain embodiments, compounds of Formula (I) provided herein exhibit improved pharmacokinetic parameters, such as bioavailablity, enhanced metabolic stability, half life and compound exposure, which allows for lower dosages and thereby reduces the risk of potential toxicity issues. By way of example only, compound 668 exhibits improved pharmacokinetic parameters. In certain embodiments, compounds of Formula (I) provided herein have significantly improved selectivity for Syk kinase over other kinases, as well as other receptors, enzymes and transporters. By way of example only, compound 734 exhibits improved Syk selectivity.

It is understood that the examples and embodiments provided herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula (I), pharmaceutically acceptable salt, pharmaceutically acceptable solvate or N-oxide thereof: 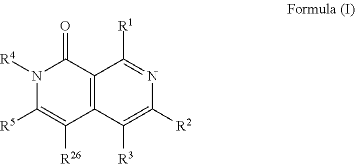
wherein:
R¹ is —NR⁶R⁷, a 4-8 membered heterocycloalkyl containing 1 to 2 heteroatoms independently selected from N, O and S or a 4-8 membered heterocycloalkyl containing 1 to 2 heteroatoms independently selected from N, O and S which is substituted with 1 to 3 substituents independently selected from hydroxyl and hydroxyl-C₁-C₆alkyl;
R² is selected from —NR⁸R¹⁰, R¹⁵, —C(O)R¹², —(CR¹²R¹²)ₙR¹⁴, —CR¹²═NOR¹², C₁-C₆alkyl, C₂-C₆alkene, phenyl, C₁₀aryl, C₁₄aryl, a 5, 6, 9, 10 or 14 membered heteroaryl containing 1 to 3 heteroatoms independently selected from N, O and S, and a 4-8 membered heterocycloalkyl containing 1 to 2 heteroatoms independently selected from N, O and S,
or R² is selected from C₁-C₆alkyl, C₂-C₆alkene, phenyl, C₁₀aryl, C₁₄aryl, a 5, 6, 9, 10 or 14 membered heteroaryl containing 1 to 3 heteroatoms independently selected from N, O and S, and a 4-8 membered heterocycloalkyl containing 1 to 2 heteroatoms independently selected from N, O and S, each of which is substituted with 1 to 3 substituents independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl;
R⁴ is H, C₁-C₆alkyl, deuterated C₁-C₆alkyl, —CD₃, C₁-C₆haloalkyl, C₂-C₆alkene, hydroxyl-C₁-C₆alkyl, R¹⁵, —(CR²⁷R²⁷)₁₋₆R¹⁴, —(CR²⁷R²⁷)(CR²⁷R²⁵)R¹¹, —(CR²⁷R²⁷)(CR²⁷R²⁵)R²⁵, —C(R²⁷R²⁵R²⁵) or —(CR²⁷R²⁷)ₙR¹¹;
each R³ and each R⁵ are independently selected from H, halogen and C₁-C₆alkyl;
R⁶ is H, phenyl, C₁₀aryl, C₁₄aryl, C₁-C₆alkyl, C₃-C₈cycloalkyl, —S(O)₂R¹³, —(CR¹²R¹²)₁₋₆R¹⁰, a 5, 6, 9, 10 or 14 membered heteroaryl containing 1 to 3 heteroatoms independently selected from N, O and S, or a 4-8 membered heterocycloalkyl containing 1 to 2 heteroatoms independently selected from N, O and S,
or R⁶ is phenyl, C₁₀aryl, C₁₄aryl, C₁-C₆alkyl, C₃-C₈cycloalkyl, a 5, 6, 9, 10 or 14 membered heteroaryl containing 1 to 3 heteroatoms independently selected from N, O and S, or a 4-8 membered heterocycloalkyl containing 1 to 2 heteroatoms independently selected from N, O and S, each of which is substituted with 1 to 3 substituents independently selected from halogen, hydroxyl, —C₁-C₆ alkyl, —C₁-C₆haloalkyl, deuterium, hydroxyl-C₁-C₆alkyl, —OR¹², R¹⁰, R¹⁵, C(O)R¹⁰, —C(O)R¹¹, —C(O)R¹², —C(O)R¹³, —C(O)R¹⁵, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)₁₋₆R¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —C(R¹²R¹²R¹⁴), —(CR¹²R¹²)ₙR¹¹, —C(O)(CR¹²R¹²)₁₋₆R¹⁴, —C(O)C(R¹²R¹²R¹⁴), —NR¹²R¹², —S(O)₂NR¹²(CR¹²R¹²)₁₋₆R¹⁴, S(O)₂NR¹²R¹², —S(O)₂R¹², C(O)C(R¹²R¹⁴)(C(R¹²R¹²))ₙR¹⁴, —(CR¹²R¹²)ₙC(O)OR¹², —C(O)N(R¹²)(CR¹²R¹²)ₙR¹¹, —(CR¹²R¹²)ₙC(O)R¹⁴, —(CR¹²R¹²)ₙC(R¹²R¹⁴)(C(R¹²R¹²))ₙR¹⁴ and —(CR¹²R¹²)ₙC(O)NR¹²(CR¹²R¹²)₁₋₆R¹⁴;
R⁷ is H or C₁-C₆ alkyl;
R⁸ is H or C₁-C₆ alkyl;
R¹⁰ is phenyl, C₁₀aryl, C₁₄aryl, a 5, 6, 9, 10 or 14 membered heteroaryl containing 1 to 3 heteroatoms independently selected from N, O and S, an N-oxide of a 5-6 membered heteroaryl containing 1-3 N heteroatoms, a 4-8 membered heterocycloalkyl containing 1 to 2 heteroatoms independently selected from N, O and S, C₃-C₈cycloalkyl or —(CR¹²R¹²)ₙR¹¹,
or R¹⁰ is phenyl, C₁₀aryl, C₁₄aryl, a 5, 6, 9, 10 or 14 membered heteroaryl containing 1 to 3 heteroatoms independently selected from N, O and S, an N-oxide of a 5-6 membered heteroaryl containing 1-3 N heteroatoms, a 4-8 membered heterocycloalkyl containing 1 to 2 heteroatoms independently selected from N, O and S, or C₃-C₈cycloalkyl, each of which is substituted with 1 to 3 substituents independently selected from halogen, hydroxyl, —NO₂, —CN, —C₁-C₆alkyl, —C₂-C₆alkene, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, R¹⁵, R¹¹, —OR¹¹, —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴, —O(CR¹²R¹²)₁₋₆R¹⁴, —O(CR¹²R¹²)ₙR¹¹, —(CR³R³)₁₋₆R¹⁴, —(CR¹²R¹²)ₙC(O)NR¹²(CR¹²R¹²)₁₋₆R¹⁴, —C(O)NR²⁷(CR¹²R¹²)ₙR¹¹, —(CR¹²R¹²)ₙC(O)NR¹²OR¹², —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₆R¹⁴, —C(O)(CR¹²R¹²)₁₋₆R¹⁴, and —C(O)C(R¹²R¹²R¹⁴);
R¹¹ is phenyl, C₁₀aryl, C₁₄aryl, a 5, 6, 9, 10 or 14 membered heteroaryl containing 1 to 4 heteroatoms independently selected from N, O and S, C₃-C₈cycloalkyl, or a 4-8 membered heterocycloalkyl containing 1 to 2 heteroatoms independently selected from N, O and S,
or R¹¹ is phenyl, C₁₀aryl, C₁₄aryl, a 5, 6, 9, 10 or 14 membered heteroaryl containing 1 to 4 heteroatoms independently selected from N, O and S, C₃-C₈cycloalkyl, or a 4-8 membered heterocycloalkyl containing 1 to 2 heteroatoms independently selected from N, O and S, each of which is substituted with 1 to 3 substituents independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴;
each R¹² is independently selected from H, C₁-C₆alkyl, hydroxyl-C₁-C₆alkyl and C₃-C₈cycloalkyl, or each R¹² is independently a C₁-C₆alkyl that together with N they are attached form a heterocycloalkyl;
R¹³ is H, C₁-C₆alkyl, halo-substituted C₁-C₆alkyl or a 4-8 membered heterocycloalkyl containing 1 to 2 heteroatoms independently selected from N, O and S;
R¹⁴ is H, halogen, hydroxyl, hydroxyl-C₁-C₆alkyl, R¹³, —OR¹³, —OR¹², —O(CR¹²R¹²)ₙOR¹³, —C(O)R¹³, —N(R¹²)₂, —NR¹²OR¹³, —CN, —C(O)N(R¹²)₂, —S(O)₂R¹³, —(CR¹²R¹²)ₙOR¹³, —C(O)R¹⁰, —OC(O)R¹³, —C(O)OR¹³, —S(O)₂N(R¹²)₂, —N(R¹²R¹⁰), —N(R¹²R¹¹), —(CR¹²R¹²)ₙN(R¹²)₂, —NR¹²C(O)(R¹²), —(CR¹²R¹²)ₙR¹³, —N(R¹²)C(O)(CR¹²R¹²)ₙOR¹³, —N(R¹²)(CR¹²R¹²)ₙOR¹³, —N(R¹²)(CR¹²R¹²)ₙR¹⁰, —C(O)N(R¹²)₂, —N(R¹²)C(O)R¹³, —N(R¹²)C(O)OR¹³, —(CR¹²R¹²)ₙR¹⁰, and R¹⁵;
R¹⁵ is 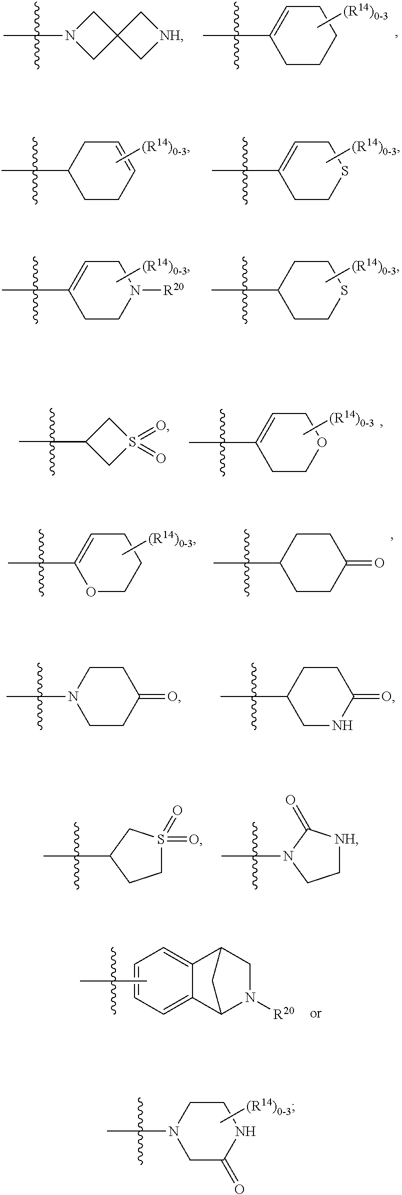
R²⁰ is H, —C₁-C₆alkyl, hydroxyl-C₁-C₆alkyl, —(CR¹²R¹²)₁₋₆R¹⁴ or —(CR¹²R¹²)ₙC(O)R¹³;
each R²⁵ is independently selected from H, hydroxyl, —C₁-C₆alkyl, —C₁-C₆haloalkyl, hydroxyl-C₁-C₆alkyl and —(CR¹²R¹²)ₙR¹⁴;
R²⁶ is H, halogen or C₁-C₆ alkyl;
each R²⁷ is independently selected from H or C₁-C₆alkyl, and
each n is independently 0, 1, 2, 3, 4, 5 or 6.

2. The compound of claim 1, wherein R¹ is —NR⁶R⁷.

3. The compound of claim 2, wherein R⁷ and R⁸ are H.

4. The compound of claim 3, wherein:
R⁶ is H, phenyl, C₁-C₆alkyl, C₃-C₈cycloalkyl, R¹⁵, —S(O)₂R¹³, —(CR¹²R¹²)₁₋₆R¹⁰; a 5, 6 or 9 membered heteroaryl containing 1 to 3 heteroatoms independently selected from N, O and S, or a 4-8 membered heterocycloalkyl containing 1 to 2 heteroatoms independently selected from N, O and S,
or R⁶ is phenyl, C₁-C₆alkyl, C₃-C₈cycloalkyl, a 5, 6 or 9 membered heteroaryl containing 1 to 3 heteroatoms independently selected from N, O and S, or a 4-8 membered heterocycloalkyl containing 1 to 2 heteroatoms independently selected from N, O and S, each of which is optionally substituted with 1 to 3 substituents independently selected from halogen, hydroxyl, —C₁-C₆alkyl, —C₁-C₆haloalkyl, deuterium, hydroxyl-C₁-C₆alkyl, —OR¹², R¹⁰, R¹⁵, C(O)R¹⁰, —C(O)R¹¹, —(CR¹²R¹²)₁₋₆R¹⁴, —(CR¹²R¹²)ₙC(O)R¹³, —(CR¹²R¹²)ₙR¹⁰, —(CR¹²R¹²)ₙC(O)R¹⁰, —O(CR¹²R¹²)ₙR¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —NR¹²R¹², —S(O)₂NR¹²(CR¹²R¹²)₁₋₆R¹⁴, —S(O)₂NR¹²R¹², —S(O)₂R¹², (CR¹²R¹²)ₙC(O)NR¹²(CR¹²R¹²)₁₋₆R¹⁴, and —C(O)N(R¹²)(CR¹²R¹²)ₙR¹¹, and
each n is independently 0, 1, 2, 3 or 4.

5. The compound of claim 4, wherein:
R⁶ is C₁-C₆alkyl or C₁-C₆alkyl substituted with 1 to 3 substituents independently selected from halogen, hydroxyl, C₁-C₆alkyl, C₁-C₆haloalkyl, hydroxyl-C₁-C₆alkyl, —OR¹², —O(CR¹²R¹²)ₙOR¹³, —C(O)R¹³, —N(R¹²)₂, —NR¹²OR¹³, —CN, —C(O)N(R¹²)₂, —S(O)₂R¹³ and R¹³;
or R⁶ is selected from 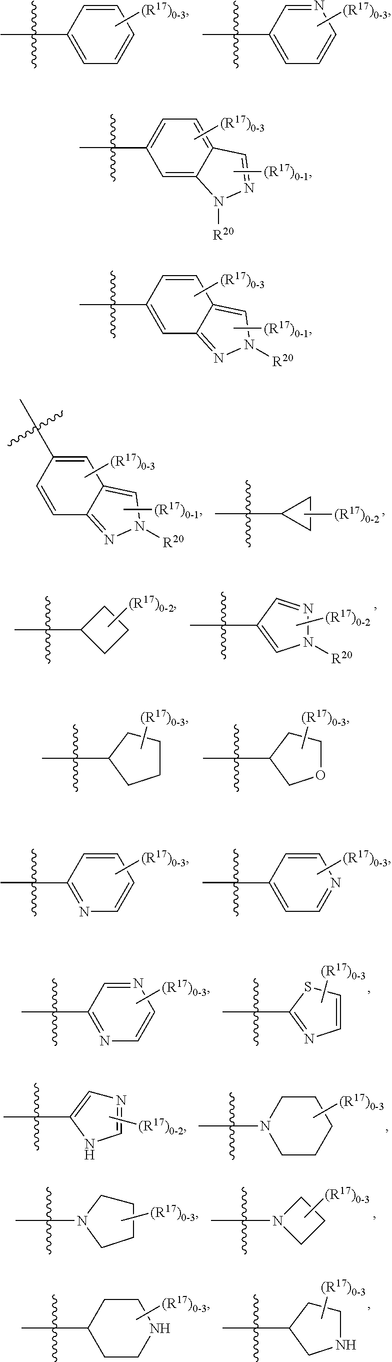 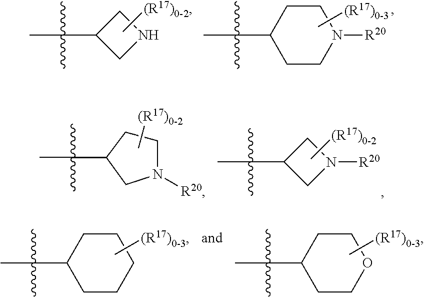
wherein
each R¹⁷ is independently selected from halogen, hydroxyl, —C₁-C₆alkyl, —C₁-C₆haloalkyl, deuterium, hydroxyl-C₁-C₆alkyl, —OR¹², R¹⁰, R¹⁵, —C(O)R¹⁰, —C(O)R¹¹, —(CR¹²R¹²)ₙR¹⁴, —(CR¹²R¹²)ₙR¹⁰, —(CR¹²R¹²)ₙC(O)R¹³, —(CR¹²R¹²)ₙR¹⁵, —(CR¹²R¹²)ₙC(O)R¹⁰, —O(CR¹²R¹²)₁₋₆R¹⁴, —O(CR¹²R¹²)ₙR¹⁰, —(CR¹²R¹²)ₙC(O)N(R¹²)₂, —C(O)N(R¹²)(CR¹²R¹²)₁₋₆R¹⁴, —(CR¹²R¹²)ₙR¹¹, —NR¹²R¹², —S(O)₂NR¹²(CR¹²R¹²)₁₋₆R¹⁴, —S(O)₂NR¹²R¹², —S(O)₂R¹², —(CR¹²R¹²)ₙC(O)NR¹²(CR¹²R¹²)₁₋₆R¹⁴, and —C(O)N(R¹²)(CR¹²R¹²)ₙR¹¹;
R²⁰ is H, —C₁-C₆alkyl, hydroxyl-C₁-C₆alkyl or —(CR¹²R¹²)ₙR¹⁰, and
each n is independently 0, 1, 2, 3 or 4.

6. The compound of claim 5, wherein R² is —NR⁸R¹⁰.

7. The compound of claim 6, wherein:
R¹⁰ is phenyl, a 5, 6 or 9 membered heteroaryl containing 1 to 3 N heteroatoms, an N-oxide of a 5-6 membered heteroaryl containing 1-3 N heteroatoms, a 4-8 membered heterocycloalkyl containing 1 to 2 heteroatoms independently selected from N, O and S, C₃-C₈cycloalkyl or —(CR¹²R¹²)ₙR¹¹,
or R¹⁰ is phenyl, a 5, 6 or 9 membered heteroaryl containing 1 to 3 N heteroatoms, an N-oxide of a 5-6 membered heteroaryl containing 1-3 N heteroatoms, a 4-8 membered heterocycloalkyl containing 1 to 2 heteroatoms independently selected from N, O and S, C₃-C₈cycloalkyl each of which is substituted with 1 to 3 substituents independently selected from halogen, hydroxyl, —NO₂, —CN, —C₁-C₆alkyl, —C₂-C₆alkene, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, R¹¹, —OR¹², —OR¹¹, —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴, —(CR¹²R¹²)ₙC(O)NR¹²OR¹², —O(CR¹²R¹²)ₙR¹⁴, —O(CR¹²R¹²)ₙR¹¹, —(CR³R³)₁₋₆R¹⁴, —(CR¹²R¹²)ₙC(O)NR¹²(CR¹²R¹²)₁₋₆R¹⁴, —C(O)NR²⁷(CR¹²R¹²)ₙR¹¹, —(CR¹²R¹²)ₙC(R¹²R¹⁴)(C(R¹²R¹²))ₙR¹⁴, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₆R¹⁴, —C(O)(CR¹²R¹²)₁₋₆R¹⁴, and —C(O)C(R¹²R¹²R¹⁴).

8. The compound of claim 7, wherein R¹⁰ is selected from 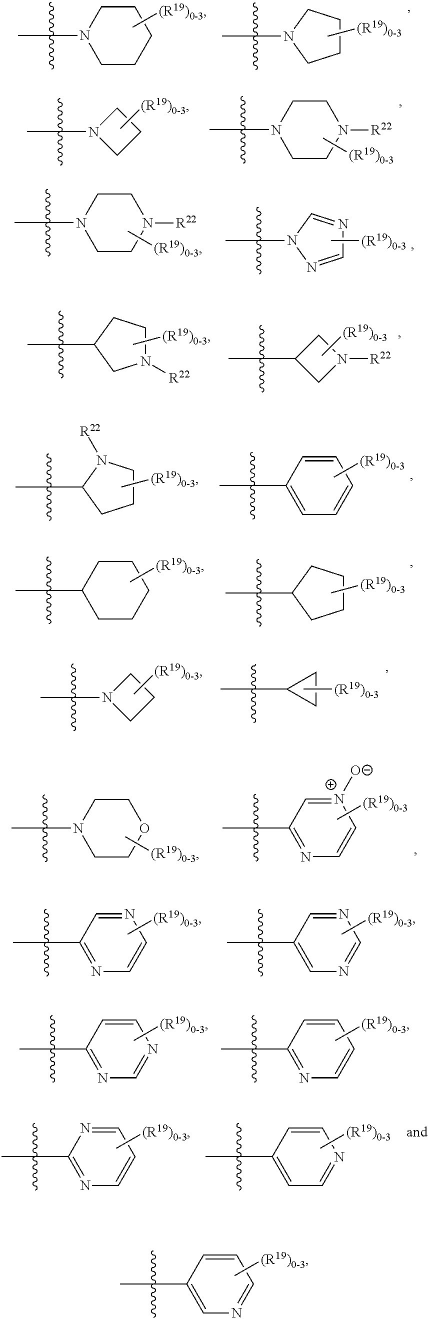
wherein
each R¹⁹ is independently selected from halogen, hydroxyl, —NO₂, —CN, —C₁-C₆alkyl, —C₂-C₆alkene, —C₁-C₆haloalkyl, hydroxyl-C₁-C₆alkyl, hydroxyl-C₁-C₆alkyl substituted with 1 to 6 deuterium, spiro attached C₃-C₈cycloalkyl, C₃-C₈cycloalkyl, R¹⁵, R¹¹, —OR¹², —OR¹¹, —C(O)R¹², —C(O)OR¹², —C(O)R¹¹, —C(O)R¹⁵, —N(R¹²)₂, —C(O)N(R¹²R¹²), —C(O)N(R¹²)(OR¹²), —(CR¹²R¹²)ₙC(O)N(R¹²R¹²), —(CR¹²R¹²)ₙC(O)OR¹², —C(R¹²R¹²R¹⁴), —(CR¹²R¹²)ₙR¹⁴, —(CR¹²R¹²)ₙC(O)NR¹²OR¹², —O(CR¹²R¹²)ₙR¹⁴, —O(CR¹²R¹²)ₙR¹¹, —(CR³R³)₁₋₄R¹⁴, —(CR¹²R¹²)ₙC(O)NR¹²(CR¹²R¹²)₁₋₆R¹⁴, —C(O)NR²⁷(CR¹²R¹²)ₙR¹¹, —(CR¹²R¹²)ₙC(R¹²R¹⁴)(C(R¹²R¹²))ₙR¹¹, —(CR¹²R¹²)ₙR¹¹, —(CR¹²R¹²)ₙC(O)R¹¹, —(CR¹²R¹²)ₙC(O)(CR¹²R¹²)₁₋₆R¹⁴, —C(O)C(R¹²R¹⁴)(C(R¹²R¹²))ₙR¹⁴, —C(R¹²R²⁵R¹³), —C(R¹²R²⁵)(CR¹²R¹²)ₙR¹⁴, —CR¹²═CR¹²(CR¹²R¹²)ₙR¹⁴, —CR²⁷═N—OR²⁷, —C(N(R²⁷)₂)═N—OR²⁷, —CR²⁷═N—O(CR¹²R¹²)₁₋₄R¹⁴, —C(O)(CR¹²R¹²)₁₋₄R¹⁴, and —C(O)C(R¹²R¹²R¹⁴);
R²² is H, —C₁-C₆alkyl, hydroxyl-C₁-C₆alkyl, —C(O)R¹², —C(O)R¹¹, R¹¹, —C(O)R¹⁵, —(CR¹²R¹²)₁₋₄R¹¹, —(CR¹²R¹²)₁₋₆R¹⁴, 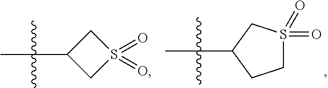
—(CR¹²R¹²)ₙC(O)N(R¹²R¹²), —(CR¹²R¹²)ₙC(O)NR¹²(CR¹²R¹²)₁₋₆R¹⁴, —C(O)C(R¹²R¹⁴)(C(R¹²R¹²))ₙR¹⁴, —(CR¹²R¹²)ₙC(O)NR¹²OR¹², —(CR¹²R¹²)ₙC(R¹²R¹⁴)(C(R¹²R¹²))ₙR¹⁴, —(CR¹²R¹²)ₙC(O)OR¹², —(CR¹²R¹²)ₙC(O)R¹¹ or —(CR¹²R¹²)ₙC(O)(CR¹²R¹²)₁₋₆R¹⁴, and
each n is independently 0, 1, 2, 3 or 4.

9. The compound of claim 8, wherein R¹⁴ is H, halogen, hydroxyl, hydroxyl-C₁-C₆alkyl, R¹³, —OR¹³, —OR¹², —O(CR¹²R¹²)ₙOR¹³, —C(O)R¹³, —N(R¹²)₂, —NR¹²OR¹³, —CN, —C(O)N(R¹²)₂, —S(O)₂R¹³—C(O)R¹⁰, —C(O)OR¹³, —S(O)₂N(R¹²)₂, N(R¹²R¹⁰), —N(R¹²R¹¹), —(CR¹²R¹²)ₙR¹³, —N(R¹²)(CR¹²R¹²)ₙOR¹³, —C(O)N(R¹²)₂, and R¹⁵.

10. The compound of claim 9, wherein R³, R⁵ and R²⁶ are H.

11. The compound of claim 10, wherein R⁴ is H, C₁-C₆alkyl, deuterated C₁-C₆alkyl, C₁-C₆haloalkyl, C₂-C₆alkene, or —CD₃.

12. The compound of claim 10, wherein R⁴ is hydroxyl-C₁-C₆alkyl.

13. The compound of claim 10, wherein R⁴ is —(CR²⁷R²⁷)₁₋₆R¹⁴, —(CR²⁷R²⁷)(CR²⁷R²⁵)R¹¹, —(CR²⁷R²⁷)(CR²⁷R²⁵)R²⁵, —C(R²⁷R²⁵R²⁵) or —(CR²⁷R²⁷)ₙR¹¹.

14. The compound of claim 13, wherein each R²⁵ is independently selected from H, hydroxyl, and hydroxyl-C₁-C₆alkyl.

15. The compound of claim 1, wherein R¹ is a 4-8 membered heterocycloalkyl containing 1 to 2 heteroatoms independently selected from N, O and S, or a 4-8 membered heterocycloalkyl containing 1 to 2 heteroatoms independently selected from N, O and S substituted with 1 to 3 substituents independently selected from hydroxyl and hydroxyl-C₁-C₆alkyl.

16. The compound of claim 15, wherein R¹ is selected from 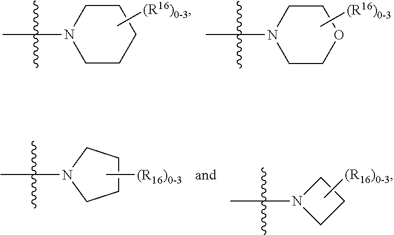
wherein each R¹⁶ is independently selected from hydroxyl and hydroxyl-C₁-C₆alkyl.

17. The compound of claim 1, wherein R² is —NR⁸R¹⁰, R¹⁵, —C(O)R¹², —(CR¹²R¹²)ₙR¹⁴, —CR¹²═NOR¹², C₁-C₆alkyl, C₂-C₆alkene, a C₁-C₆alkyl substituted with 1 to 3 substituents independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl or a C₂-C₆alkene substituted with 1 to 3 substituents independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl.

18. The compound of claim 1, wherein:
R² is selected from phenyl, a 5, 6, or 9 membered heteroaryl containing 1 to 3 N heteroatoms, and a 4-8 membered heterocycloalkyl containing 1 to 2 heteroatoms independently selected from N, O and S,
or R² is selected from phenyl, a 5, 6, or 9 membered heteroaryl containing 1 to 3 N heteroatoms, and a 4-8 membered heterocycloalkyl containing 1 to 2 heteroatoms independently selected from N, O and S each of which is substituted with 1 to 3 substituents independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl, and
each n is independently 0, 1, 2, 3 or 4.

19. The compound of claim 18, wherein R² is selected from 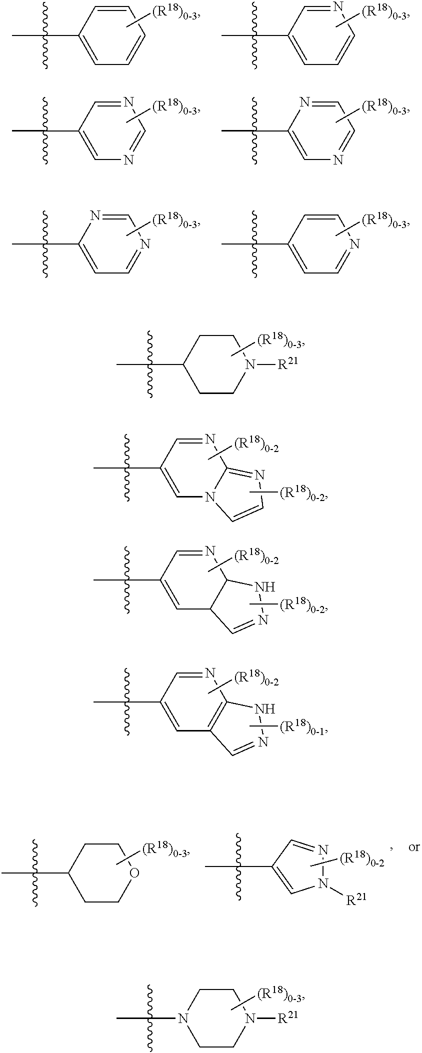
wherein
each R¹⁸ is independently selected from —OR¹², —OR¹⁰, —C(O)OR¹², —C(O)R¹⁰, —N(R¹²)₂, —(CR¹²R¹²)ₙR¹⁴, —C₁-C₆alkyl and hydroxyl-C₁-C₆alkyl;
each R¹² is independently selected from H, —C₁-C₆alkyl and C₃-C₈cycloalkyl;
R¹⁴ is —OR¹²;
R²¹ is H, C₁-C₆alkyl, —(CR¹²R¹²)₁₋₄R¹⁴ or hydroxyl-C₁-C₆alkyl, and
each n is independently 0, 1, 2, 3 or 4.

20. The compound of claim 1, wherein R¹⁰ is —(CR¹²R¹²)ₙR¹¹.

21. The compound of claim 20, wherein R¹¹ is a 4-8 membered heterocycloalkyl containing 1 to 2 heteroatoms independently selected from N, O and S or a 4-8 membered heterocycloalkyl containing 1 to 2 heteroatoms independently selected from N, O and S substituted with 1 to 3 substituents independently selected from halogen, hydroxyl, —C₁-C₆alkyl, C₃-C₈cycloalkyl, hydroxyl-C₁-C₆alkyl and —(CR¹²R¹²)ₙR¹⁴, and
each n is independently 0, 1, 2, 3, 4, 5 or 6.

22. The compound of claim 21, wherein R¹¹ is selected from 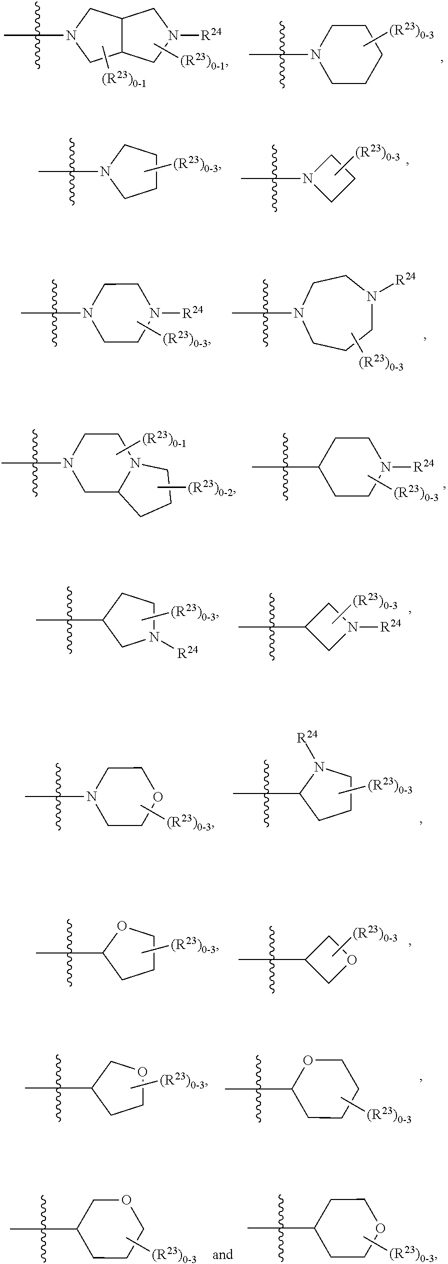
wherein
each R²³ is independently selected from halogen, hydroxyl, —C₁-C₆alkyl, C₃-C₈cycloalkyl, hydroxyl-C₁-C₆alkyl and —(CR¹²R¹²)ₙR¹⁴;
R²⁴ is H, —C₁-C₆alkyl, hydroxyl-C₁-C₆alkyl or —(CR¹²R¹²)₁₋₄R¹⁴, and
each n is independently 0, 1, 2, 3 or 4.

23. The compound of claim 1, wherein R¹¹ is a C₃-C₈cycloalkyl or a C₃-C₈cycloalkyl substituted with 1 to 3 substituents independently selected from halogen, hydroxyl, —C₁-C₆alkyl, C₃-C₈cycloalkyl, hydroxyl-C₁-C₆alkyl and —(CR¹²R¹²)ₙR¹⁴ and
each n is independently 0, 1, 2, 3, 4, 5 or 6.

24. The compound of claim 23, wherein R¹¹ is selected from 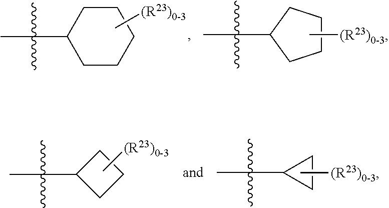
wherein,
each R²³ is independently selected from halogen, hydroxyl, —C₁-C₆alkyl, C₃-C₈cycloalkyl, hydroxyl-C₁-C₆alkyl and —(CR¹²R¹²)ₙR¹⁴, and
each n is independently 0, 1, 2, 3 or 4.

25. The compound of claim 1, wherein R¹¹ is a 5, 6 or 9 membered heteroaryl containing 1 to 4 heteroatoms independently selected from N, O and S, or a 5, 6 or 9 membered heteroaryl containing 1 to 4 heteroatoms independently selected from N, O and S substituted with 1 to 3 substituents independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴ and
each n is independently 0, 1, 2, 3, 4, 5 or 6.

26. The compound of claim 25, wherein R¹¹ is selected from 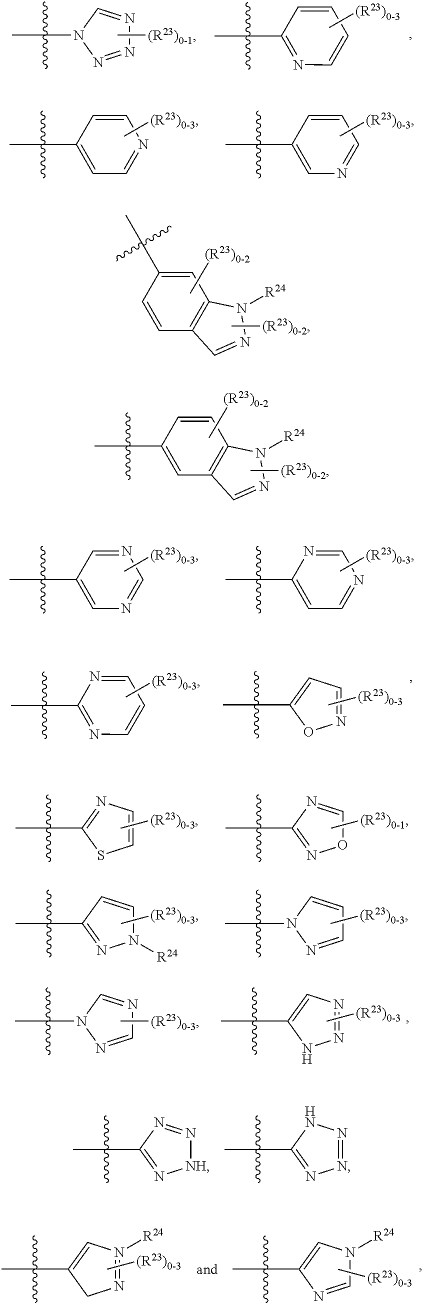
wherein
each R²³ is independently selected from halogen, hydroxyl, —C₁-C₆alkyl, halo-substituted C₁-C₆alkyl, C₃-C₈cycloalkyl, hydroxyl-C₁-C₆alkyl and —(CR¹²R¹²)ₙR¹⁴;
R²⁴ is H, —C₁-C₆alkyl, hydroxyl-C₁-C₆alkyl or —(CR¹²R¹²)₁₄R¹⁴, and
each n is independently 1, 2, 3 or 4.

27. The compound of claim 1, wherein R¹¹ is 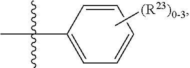
wherein
each R²³ is independently selected from halogen, hydroxyl, —C₁-C₆alkyl, C₃-C₈cycloalkyl, hydroxyl-C₁-C₆alkyl and —(CR¹²R¹²)ₙR¹⁴, and each n is independently 0, 1, 2, 3 or 4.

28. The compound of claim 1 selected from:
8-amino-2-methyl-6-(pyridin-2-ylamino)-1,2-dihydro-2,7-naphthyridin-1-one;
6-[(1-amino-7-methyl-8-oxo-7,8-dihydro-2,7-naphthyridin-3-yl)amino]pyridine-3-carbonitrile;
6-[(1-amino-7-methyl-8-oxo-7,8-dihydro-2,7-naphthyridin-3-yl)amino]pyridine-3-carboxamide;
8-amino-2-methyl-6-(pyrazin-2-ylamino)-1,2-dihydro-2,7-naphthyridin-1-one;
8-amino-2-benzyl-6-(pyrazin-2-ylamino)-1,2-dihydro-2,7-naphthyridin-1-one;
6-{3-methyl-1H-pyrazolo[3,4-b]pyridin-5-yl}-8-{[4-(morpholin-4-yl)phenyl]amino}-1,2-dihydro-2,7-naphthyridin-1-one;
8-{[3-fluoro-4-(2-methylpiperidin-4-yl)phenyl]amino}-6-(pyrimidin-5-yl)-1,2-dihydro-2,7-naphthyridin-1-one;
8-{[3-(4-ethylpiperazin-1-yl)-1H-indazol-6-yl]amino}-6-(pyrimidin-5-yl)-1,2-dihydro-2,7-naphthyridin-1-one;
8-{[3-(4-ethylpiperazin-1-yl)-1H-indazol-6-yl]amino}-6-(6-methylpyridin-3-yl)-1,2-dihydro-2,7-naphthyridin-1-one;
8-({3-[(4-ethylpiperazin-1-yl)methyl]-1H-indazol-6-yl}amino)-6-(pyrimidin-5-yl)-1,2-dihydro-2,7-naphthyridin-1-one;
8-({3-[(4-ethylpiperazin-1-yl)methyl]-1H-indazol-6-yl}amino)-6-(6-methylpyridin-3-yl)-1,2-dihydro-2,7-naphthyridin-1-one;
8-({3-[(4-ethylpiperazin-1-yl)methyl]-1H-indazol-6-yl}amino)-6-(2-methoxypyrimidin-5-yl)-1,2-dihydro-2,7-naphthyridin-1-one;
3-[4-(2-methyl-4-{[3-(6-methylpyridin-3-yl)-8-oxo-7,8-dihydro-2,7-naphthyridin-1-yl]amino}-phenyl)piperidin-1-yl]propanenitrile;
6-(6-methylpyridin-3-yl)-8-({1-[2-(morpholin-4-yl)ethyl]-1H-pyrazol-4-yl}amino)-1,2-dihydro-2,7-naphthyridin-1-one;
8-({4-[1-(2,3-dihydroxypropyl)piperidin-4-yl]phenyl}amino)-6-(6-methoxypyrazin-2-yl)-1,2-dihydro-2,7-naphthyridin-1-one;
3-[4-(4-{[3-(6-methoxypyrazin-2-yl)-8-oxo-7,8-dihydro-2,7-naphthyridin-1-yl]amino}-2-methylphenyl)piperidin-1-yl]propanenitrile;
8-{[4-(morpholin-4-yl)phenyl]amino}-6-(pyrazin-2-ylamino)-1,2-dihydro-2,7-naphthyridin-1-one;
8-{[4-(morpholin-4-yl)phenyl]amino}-6-(pyridin-2-ylamino)-1,2-dihydro-2,7-naphthyridin-1-one;
6-[(3-methylpyridin-2-yl)amino]-8-{[4-(morpholin-4-yl)phenyl]amino}-1,2-dihydro-2,7-naphthyridin-1-one;
6-[(5-methylpyridin-2-yl)amino]-8-{[4-(morpholin-4-yl)phenyl]amino}-1,2-dihydro-2,7-naphthyridin-1-one;
6-[(6-methylpyridin-2-yl)amino]-8-{[4-(morpholin-4-yl)phenyl]amino}-1,2-dihydro-2,7-naphthyridin-1-one;
6-(6-methoxypyrazin-2-yl)-8-[(4-{2-[4-(propan-2-yl)piperazin-1-yl]ethyl}phenyl)amino]-1,2-dihydro-2,7-naphthyridin-1-one;
8-{[4-(2-{6,9-diazaspiro[4.5]decan-9-yl}ethyl)phenyl]amino}-6-(6-methoxypyrazin-2-yl)-1,2-dihydro-2,7-naphthyridin-1-one;
8-({4-[2-(4-methoxypiperidin-1-yl)ethyl]phenyl}amino)-6-(6-methoxypyrazin-2-yl)-1,2-dihydro-2,7-naphthyridin-1-one;
6-(6-methoxypyrazin-2-yl)-8-({4-[2-(1H-1,2,4-triazol-1-yl)ethyl]phenyl}amino)-1,2-dihydro-2,7-naphthyridin-1-one;
6-(6-methoxypyrazin-2-yl)-8-({4-[3-(morpholin-4-yl)-3-oxopropyl]phenyl}amino)-1,2-dihydro-2,7-naphthyridin-1-one;
8-({4-[1-(3-methoxypropanoyl)piperidin-4-yl]phenyl}amino)-6-(6-methoxypyrazin-2-yl)-1,2-dihydro-2,7-naphthyridin-1-one;
8-[(4-{1-[2-(2-methoxyethoxy)acetyl]piperidin-4-yl}phenyl)amino]-6-(6-methoxypyrazin-2-yl)-1,2-dihydro-2,7-naphthyridin-1-one;
8-({4-[1-(2-ethoxyacetyl)piperidin-4-yl]phenyl}amino)-6-(6-methoxypyrazin-2-yl)-1,2-dihydro-2,7-naphthyridin-1-one;
2-{4-[(3-{imidazo[1,2-a]pyrimidin-6-yl}-8-oxo-7,8-dihydro-2,7-naphthyridin-1-yl)amino]phenyl}-N,2-dimethylpropanamide;
6-{imidazo[1,2-a]pyrimidin-6-yl}-8-({4-[1-(2-methoxyethyl)piperidin-4-yl]-3-methylphenyl}amino)-1,2-dihydro-2,7-naphthyridin-1-one;
6-(2-aminopyrimidin-5-yl)-2-methyl-8-[(1-methylpiperidin-4-yl)amino]-1,2-dihydro-2,7-naphthyridin-1-one;
6-(2-aminopyrimidin-5-yl)-2-(2-hydroxyethyl)-8-(propan-2-ylamino)-1,2-dihydro-2,7-naphthyridin-1-one;
8-({3-fluoro-4-[1-(3-methoxypropyl)piperidin-4-yl]phenyl}amino)-6-(pyrimidin-5-yl)-1,2-dihydro-2,7-naphthyridin-1-one;
8-{[3-fluoro-4-(piperidin-4-yl)phenyl]amino}-6-(pyrimidin-5-yl)-1,2-dihydro-2,7-naphthyridin-1-one;
8-({3-fluoro-4-[1-(3-methoxypropyl)piperidin-4-yl]phenyl}amino)-6-(2-methoxypyrimidin-5-yl)-1,2-dihydro-2,7-naphthyridin-1-one;
8-{[3-fluoro-4-(piperidin-4-yl)phenyl]amino}-6-(2-methoxypyrimidin-5-yl)-1,2-dihydro-2,7-naphthyridin-1-one;
8-{[3-(4-ethylpiperazin-1-yl)-1H-indazol-6-yl]amino}-6-{5-[(morpholin-4-yl)carbonyl]pyridin-3-yl}-1,2-dihydro-2,7-naphthyridin-1-one;
8-({3-[(4-ethylpiperazin-1-yl)methyl]-1H-indazol-6-yl}amino)-6-{5-[(morpholin-4-yl)carbonyl]pyridin-3-yl}-1,2-dihydro-2,7-naphthyridin-1-one;
8-{[4-(1-cyclopropylpiperidin-4-yl)-3-methylphenyl]amino}-6-{5-[(morpholin-4-yl)carbonyl]pyridin-3-yl}-1,2-dihydro-2,7-naphthyridin-1-one;
8-({3-[(dimethylamino)methyl]-1H-indazol-6-yl}amino)-6-{5-[(morpholin-4-yl)carbonyl]pyridin-3-yl}-1,2-dihydro-2,7-naphthyridin-1-one;
8-({4-[2-(4-ethyl-3-methylpiperazin-1-yl)ethyl]phenyl}amino)-6-(6-methoxypyrazin-2-yl)-1,2-dihydro-2,7-naphthyridin-1-one;
6-(pyrimidin-5-yl)-8-({4-[2-(1H-1,2,4-triazol-1-yl)ethyl]phenyl}amino)-1,2-dihydro-2,7-naphthyridin-1-one;
6-(2-aminopyrimidin-5-yl)-8-(propan-2-ylamino)-1,2-dihydro-2,7-naphthyridin-1-one;
6-(2-aminopyrimidin-5-yl)-2-methyl-8-{[2-(morpholin-4-yl)ethyl]amino}-1,2-dihydro-2,7-naphthyridin-1-one;
6-(2-aminopyrimidin-5-yl)-8-(cyclopropylamino)-2-ethyl-1,2-dihydro-2,7-naphthyridin-1-one;
6-(2-aminopyrimidin-5-yl)-8-(cyclopropylamino)-2-methyl-1,2-dihydro-2,7-naphthyridin-1-one;
6-[(1-amino-7-methyl-8-oxo-7,8-dihydro-2,7-naphthyridin-3-yl)amino]-N-propylpyridine-3-carboxamide;
6-(3,6-dimethylpyrazin-2-yl)-8-{[4-(morpholin-4-yl)phenyl]amino}-1,2-dihydro-2,7-naphthyridin-1-one;
6-(6-methoxypyrazin-2-yl)-8-({4-[3-(morpholin-4-yl)propyl]phenyl}amino)-1,2-dihydro-2,7-naphthyridin-1-one;
8-({4-[3-(4-ethylpiperazin-1-yl)propyl]phenyl}amino)-6-(6-methoxypyrazin-2-yl)-1,2-dihydro-2,7-naphthyridin-1-one;
3-[4-(4-{[3-(6-methoxypyrazin-2-yl)-8-oxo-7,8-dihydro-2,7-naphthyridin-1-yl]amino}-2-methylphenyl)piperidin-1-yl]-1$1̂{6}-thietane-1,1-dione;
8-{[4-(azetidin-3-yl)phenyl]amino}-6-(pyrimidin-5-yl)-1,2-dihydro-2,7-naphthyridin-1-one;
8-{[4-(azetidin-3-yl)phenyl]amino}-6-(2-methoxypyrimidin-5-yl)-1,2-dihydro-2,7-naphthyridin-1-one;
8-{[4-(azetidin-3-yl)phenyl]amino}-6-(6-methylpyridin-3-yl)-1,2-dihydro-2,7-naphthyridin-1-one;
8-({4-[1-(2-methanesulfonylethyl)piperidin-4-yl]-3-methylphenyl}amino)-6-(6-methoxypyrazin-2-yl)-1,2-dihydro-2,7-naphthyridin-1-one;
8-({4-[1-(3-methoxypropanoyl)piperidin-4-yl]-3-methylphenyl}amino)-6-(6-methoxypyrazin-2-yl)-1,2-dihydro-2,7-naphthyridin-1-one;
8-({4-[2-(4,4-difluoropiperidin-1-yl)ethyl]phenyl}amino)-6-(6-methoxypyrazin-2-yl)-1,2-dihydro-2,7-naphthyridin-1-one;
8-({4-[2-(4-acetylpiperazin-1-yl)ethyl]phenyl}amino)-6-(6-methoxypyrazin-2-yl)-1,2-dihydro-2,7-naphthyridin-1-one;
2-[4-(4-{[3-(6-methoxypyrazin-2-yl)-8-oxo-7,8-dihydro-2,7-naphthyridin-1-yl]amino}-2-methylphenyl)piperidin-1-yl]acetonitrile;
2-[4-(4-{[3-(6-methoxypyrazin-2-yl)-8-oxo-7,8-dihydro-2,7-naphthyridin-1-yl]amino}-2-methylphenyl)piperidin-1-yl]acetamide;
2-[4-(4-{[3-(6-methoxypyrazin-2-yl)-8-oxo-7,8-dihydro-2,7-naphthyridin-1-yl]amino}-2-methylphenyl)piperidin-1-yl]-N-methylacetamide;
2-[4-(4-{[3-(6-methoxypyrazin-2-yl)-8-oxo-7,8-dihydro-2,7-naphthyridin-1-yl]amino}-2-methylphenyl)piperidin-1-yl]propanamide;
8-{[4-(1-ethylpiperidin-4-yl)-3-methylphenyl]amino}-6-(6-methoxypyrazin-2-yl)-1,2-dihydro-2,7-naphthyridin-1-one;
8-{[4-(1-methylazetidin-3-yl)phenyl]amino}-6-(pyrimidin-5-yl)-1,2-dihydro-2,7-naphthyridin-1-one;
6-(2-methoxypyrimidin-5-yl)-8-{[4-(1-methylazetidin-3-yl)phenyl]amino}-1,2-dihydro-2,7-naphthyridin-1-one;
8-{[4-(1-methylazetidin-3-yl)phenyl]amino}-6-(6-methylpyridin-3-yl)-1,2-dihydro-2,7-naphthyridin-1-one;
6-[(5-fluoropyridin-2-yl)amino]-8-{[4-(morpholin-4-yl)phenyl]amino}-1,2-dihydro-2,7-naphthyridin-1-one;
8-{[4-(morpholin-4-yl)phenyl]amino}-6-{[5-(trifluoromethyl)pyridin-2-yl]amino}-1,2-dihydro-2,7-naphthyridin-1-one;
6-{[(1-[4-(morpholin-4-yl)phenyl]amino}-8-oxo-7,8-dihydro-2,7-naphthyridin-3-yl)amino]pyridine-3-carbonitrile;
6-{[(1-[4-(morpholin-4-yl)phenyl]amino}-8-oxo-7,8-dihydro-2,7-naphthyridin-3-yl)amino]pyridine-3-carboxamide;
8-{[4-(morpholin-4-yl)phenyl]amino}-6-[(5-nitropyridin-2-yl)amino]-1,2-dihydro-2,7-naphthyridin-1-one;
8-({3-fluoro-4-[1-(2-methoxyethyl)piperidin-4-yl]phenyl}amino)-6-(pyrimidin-5-yl)-1,2-dihydro-2,7-naphthyridin-1-one;
8-{[3-(4-ethylpiperazin-1-yl)-1H-indazol-6-yl]amino}-6-(6-methoxypyrazin-2-yl)-1,2-dihydro-2,7-naphthyridin-1-one;
8-{[4-(1-cyclopropylpiperidin-4-yl)-3-methylphenyl]amino}-6-(6-methoxypyrazin-2-yl)-1,2-dihydro-2,7-naphthyridin-1-one;
ethyl 6-[(1-{[4-(morpholin-4-yl)phenyl]amino}-8-oxo-7,8-dihydro-2,7-naphthyridin-3-yl)amino]pyridine-3-carboxylate;
6-[(5-chloropyridin-2-yl)amino]-8-{[4-(morpholin-4-yl)phenyl]amino}-1,2-dihydro-2,7-naphthyridin-1-one;
6-({5-[2-(morpholin-4-yl)ethyl]pyridin-2-yl}amino)-8-{[4-(morpholin-4-yl)phenyl]amino}-1,2-dihydro-2,7-naphthyridin-1-one;
6-(2-aminopyrimidin-5-yl)-4-fluoro-8-{[4-(morpholin-4-yl)phenyl]amino}-1,2-dihydro-2,7-naphthyridin-1-one;
4-fluoro-8-{[4-(morpholin-4-yl)phenyl]amino}-6-(pyrimidin-5-yl)-1,2-dihydro-2,7-naphthyridin-1-one;
4-fluoro-6-(2-methoxypyrimidin-5-yl)-8-{[4-(morpholin-4-yl)phenyl]amino}-1,2-dihydro-2,7-naphthyridin-1-one;
4-fluoro-6-(6-methylpyridin-3-yl)-8-{[4-(morpholin-4-yl)phenyl]amino}-1,2-dihydro-2,7-naphthyridin-1-one;
4-fluoro-6-(6-methoxypyrazin-2-yl)-8-{[4-(morpholin-4-yl)phenyl]amino}-1,2-dihydro-2,7-naphthyridin-1-one;
8-({4-[1-(3-methoxypropyl)piperidin-4-yl]-3-methylphenyl}amino)-6-(6-methoxypyrazin-2-yl)-1,2-dihydro-2,7-naphthyridin-1-one;
8-({4-[1-(2-hydroxy-3-methoxypropyl)piperidin-4-yl]-3-methylphenyl}amino)-6-(6-methoxypyrazin-2-yl)-1,2-dihydro-2,7-naphthyridin-1-one;
8-({4-[1-(2-hydroxyethyl)piperidin-4-yl]-3-methylphenyl}amino)-6-(6-methoxypyrazin-2-yl)-1,2-dihydro-2,7-naphthyridin-1-one;
8-{[4-(1-acetylpiperidin-4-yl)-3-methylphenyl]amino}-6-(6-methoxypyrazin-2-yl)-1,2-dihydro-2,7-naphthyridin-1-one;
8-{[4-(azetidin-3-yl)phenyl]amino}-6-(6-methoxypyrazin-2-yl)-1,2-dihydro-2,7-naphthyridin-1-one;
6-(6-methoxypyrazin-2-yl)-8-{[4-(1-methylazetidin-3-yl)phenyl]amino}-1,2-dihydro-2,7-naphthyridin-1-one;
8-({4-[1-(3-fluoropropyl)piperidin-4-yl]-3-methylphenyl}amino)-6-(6-methylpyridin-3-yl)-1,2-dihydro-2,7-naphthyridin-1-one;
8-({4-[1-(2-hydroxyethyl)azetidin-3-yl]phenyl}amino)-6-(6-methoxypyrazin-2-yl)-1,2-dihydro-2,7-naphthyridin-1-one;
8-({4-[1-(2-methanesulfonylethyl)azetidin-3-yl]phenyl}amino)-6-(6-methoxypyrazin-2-yl)-1,2-dihydro-2,7-naphthyridin-1-one;
8-{[4-(1-cyclopropylpiperidin-4-yl)-3-methylphenyl]amino}-6-(6-methylpyridin-3-yl)-1,2-dihydro-2,7-naphthyridin-1-one;
8-({4-[1-(2-methoxyethyl)piperidin-4-yl]-3-methylphenyl}amino)-6-(6-methylpyridin-3-yl)-1,2-dihydro-2,7-naphthyridin-1-one;
6-[6-(cyclopropylamino)pyrazin-2-yl]-8-{[4-(morpholin-4-yl)phenyl]amino}-1,2-dihydro-2,7-naphthyridin-1-one;
8-{[4-(1-acetylazetidin-3-yl)phenyl]amino}-6-(6-methoxypyrazin-2-yl)-1,2-dihydro-2,7-naphthyridin-1-one;
8-({4-[1-(2-methoxyethyl)azetidin-3-yl]phenyl}amino)-6-(6-methoxypyrazin-2-yl)-1,2-dihydro-2,7-naphthyridin-1-one;
6-(2-methoxypyrimidin-5-yl)-8-({4-[2-(3-oxopiperazin-1-yl)ethyl]phenyl}amino)-1,2-dihydro-2,7-naphthyridin-1-one;
6-(6-methylpyridin-3-yl)-8-({4-[2-(3-oxopiperazin-1-yl)ethyl]phenyl}amino)-1,2-dihydro-2,7-naphthyridin-1-one;
3-[3-(4-{[3-(6-methoxypyrazin-2-yl)-8-oxo-7,8-dihydro-2,7-naphthyridin-1-yl]amino}-phenyl)azetidin-1-yl]propanenitrile;
3-[4-(4-{[3-(5-amino-6-methoxypyrazin-2-yl)-8-oxo-7,8-dihydro-2,7-naphthyridin-1-yl]amino}-phenyl)piperidin-1-yl]propanenitrile;
8-({4-[1-(2-methoxyacetyl)piperidin-4-yl]-3-methylphenyl}amino)-6-(6-methoxypyrazin-2-yl)-1,2-dihydro-2,7-naphthyridin-1-one;
8-({4-[1-(2-ethoxyacetyl)piperidin-4-yl]-3-methylphenyl}amino)-6-(6-methoxypyrazin-2-yl)-1,2-dihydro-2,7-naphthyridin-1-one;
6-(2-amino-6-methylpyrimidin-4-yl)-8-({4-[1-(3-methoxypropanoyl)piperidin-4-yl]-3-methylphenyl}amino)-1,2-dihydro-2,7-naphthyridin-1-one;
8-({4-[1-(2-amino-2-methylpropanoyl)piperidin-4-yl]-3-methylphenyl}amino)-6-(6-methoxypyrazin-2-yl)-1,2-dihydro-2,7-naphthyridin-1-one;
6-(2-aminopyrimidin-4-yl)-8-{[4-(morpholin-4-yl)phenyl]amino}-1,2-dihydro-2,7-naphthyridin-1-one;
6-(6-methoxypyrazin-2-yl)-8-({4-[2-(piperazin-1-yl)ethyl]phenyl}amino)-1,2-dihydro-2,7-naphthyridin-1-one;
2-{4-[2-methyl-4-({3-[(5-methylpyridin-2-yl)amino]-8-oxo-7,8-dihydro-2,7-naphthyridin-1-yl}amino)phenyl]piperidin-1-yl}acetamide;
8-({4-[2-(4-acetylpiperazin-1-yl)ethyl]phenyl}amino)-6-[(5-methylpyridin-2-yl)amino]-1,2-dihydro-2,7-naphthyridin-1-one;
6-(6-methoxypyrazin-2-yl)-8-{[4-(2-{octahydropyrrolo[3,4-c]pyrrol-2-yl}ethyl)phenyl]amino}-1,2-dihydro-2,7-naphthyridin-1-one;
6-(6-methoxypyrazin-2-yl)-8-({4-[2-(4-methyl-1,4-diazepan-1-yl)ethyl]phenyl}amino)-1,2-dihydro-2,7-naphthyridin-1-one;
8-({4-[2-(4-methyl-1,4-diazepan-1-yl)ethyl]phenyl}amino)-6-[(5-methylpyridin-2-yl)amino]-1,2-dihydro-2,7-naphthyridin-1-one;
8-{[4-(1-cyclopropylpiperidin-4-yl)-3-methylphenyl]amino}-6-[1-(2-methylpropyl)-1H-pyrazol-4-yl]-1,2-dihydro-2,7-naphthyridin-1-one;
6-(6-methoxypyrazin-2-yl)-8-{[3-methyl-4-(1-propylpiperidin-4-yl)phenyl]amino}-1,2-dihydro-2,7-naphthyridin-1-one;
6-(6-methoxypyrazin-2-yl)-8-({4-[4-(morpholin-4-yl)cyclohexyl]phenyl}amino)-1,2-dihydro-2,7-naphthyridin-1-one;
6-(6-methoxypyrazin-2-yl)-8-({4-[2-(3-oxopiperazin-1-yl)ethyl]phenyl}amino)-1,2-dihydro-2,7-naphthyridin-1-one;
6-(6-methoxypyrazin-2-yl)-8-[(3-methyl-4-{1-[2-oxo-2-(pyrrolidin-1-yl)ethyl]piperidin-4-yl}phenyl)amino]-1,2-dihydro-2,7-naphthyridin-1-one;
methyl 2-[4-(4-{[3-(6-methoxypyrazin-2-yl)-8-oxo-7,8-dihydro-2,7-naphthyridin-1-yl]amino}-2-methylphenyl)piperidin-1-yl]acetate;
3-[4-(4-{[3-(6-methoxypyrazin-2-yl)-8-oxo-7,8-dihydro-2,7-naphthyridin-1-yl]amino}-2-methylphenyl)piperidin-1-yl]propanamide;
3-[4-(4-{[3-(6-methoxypyrazin-2-yl)-8-oxo-7,8-dihydro-2,7-naphthyridin-1-yl]amino}-2-methylphenyl)piperidin-1-yl]-N,N-dimethylpropanamide;
6-[(5-methylpyridin-2-yl)amino]-8-{[4-(2-octahydropyrrolo[3,4-c]pyrrol-2-yl ethyl)phenyl]amino}-1,2-dihydro-2,7-naphthyridin-1-one;
8-({4-[2-(4-methyl-1,4-diazepan-1-yl)ethyl]phenyl}amino)-6-(pyrimidin-5-yl)-1,2-dihydro-2,7-naphthyridin-1-one;
6-(2-methoxypyrimidin-5-yl)-8-({4-[2-(4-methyl-1,4-diazepan-1-yl)ethyl]phenyl}amino)-1,2-dihydro-2,7-naphthyridin-1-one;
8-({4-[2-(4-methyl-1,4-diazepan-1-yl)ethyl]phenyl}amino)-6-(6-methylpyridin-3-yl)-1,2-dihydro-2,7-naphthyridin-1-one;
8-({4-[2-(4-ethylpiperazin-1-yl)ethyl]phenyl}amino)-6-[(5-methylpyridin-2-yl)amino]-1,2-dihydro-2,7-naphthyridin-1-one;
8-({4-[2-(4-ethylpiperazin-1-yl)ethyl]phenyl}amino)-6-(pyridin-2-ylamino)-1,2-dihydro-2,7-naphthyridin-1-one;
8-{[4-(1-cyclopropylpiperidin-4-yl)-3-methylphenyl]amino}-6-[(5-methylpyridin-2-yl)amino]-1,2-dihydro-2,7-naphthyridin-1-one;
6-[(5-chloropyridin-2-yl)amino]-8-{[4-(1-cyclopropylpiperidin-4-yl)-3-methylphenyl]amino}-1,2-dihydro-2,7-naphthyridin-1-one;
8-{[4-(1-cyclopropylpiperidin-4-yl)-3-methylphenyl]amino}-6-(2-methylpyridin-4-yl)-1,2-dihydro-2,7-naphthyridin-1-one;
methyl 3-[4-(4-{[3-(6-methoxypyrazin-2-yl)-8-oxo-7,8-dihydro-2,7-naphthyridin-1-yl]amino}-2-methylphenyl)piperidin-1-yl]propanoate;
3-[4-(4-{[3-(6-methoxypyrazin-2-yl)-8-oxo-7,8-dihydro-2,7-naphthyridin-1-yl]amino}-2-methylphenyl)piperidin-1-yl]-N-methylpropanamide;
6-(6-methoxypyrazin-2-yl)-8-[(3-methyl-4-{1-[3-(morpholin-4-yl)-3-oxopropyl]piperidin-4-yl}phenyl)amino]-1,2-dihydro-2,7-naphthyridin-1-one;
3-[4-(4-{[3-(6-methoxypyrazin-2-yl)-8-oxo-7,8-dihydro-2,7-naphthyridin-1-yl]amino}-2-methylphenyl)piperidin-1-yl]propanoic acid;
8-{[4-(1-cyclopropylpiperidin-4-yl)-3-methylphenyl]amino}-6-[(5-methylpyrazin-2-yl)amino]-1,2-dihydro-2,7-naphthyridin-1-one;
8-({4-[4-(morpholin-4-yl)cyclohexyl]phenyl}amino)-6-(pyrimidin-5-yl)-1,2-dihydro-2,7-naphthyridin-1-one;
8-[(4-{1-[(2R)-2-methoxypropanoyl]piperidin-4-yl}-3-methylphenyl)amino]-6-(6-methoxypyrazin-2-yl)-1,2-dihydro-2,7-naphthyridin-1-one;
8-[(4-{1-[(2S)-2-methoxypropanoyl]piperidin-4-yl}-3-methylphenyl)amino]-6-(6-methoxypyrazin-2-yl)-1,2-dihydro-2,7-naphthyridin-1-one;
N-(2-methoxyethyl)-4-{[3-(6-methoxypyrazin-2-yl)-8-oxo-7,8-dihydro-2,7-naphthyridin-1-yl]amino}benzamide;
6-{[1-({4-[2-(4-ethylpiperazin-1-yl)ethyl]phenyl}amino)-8-oxo-7,8-dihydro-2,7-naphthyridin-3-yl]amino}pyridine-3-carboxamide;
6-{[1-({4-[2-(4-ethylpiperazin-1-yl)ethyl]phenyl}amino)-8-oxo-7,8-dihydro-2,7-naphthyridin-3-yl]amino}pyridine-3-carbonitrile;
6-[(5-chloropyridin-2-yl)amino]-8-({4-[2-(4-ethylpiperazin-1-yl)ethyl]phenyl}amino)-1,2-dihydro-2,7-naphthyridin-1-one;
8-({4-[2-(4-ethylpiperazin-1-yl)ethyl]phenyl}amino)-4-fluoro-6-(pyrimidin-5-yl)-1,2-dihydro-2,7-naphthyridin-1-one;
8-({4-[2-(4-ethylpiperazin-1-yl)ethyl]phenyl}amino)-4-fluoro-6-(2-methoxypyrimidin-5-yl)-1,2-dihydro-2,7-naphthyridin-1-one;
8-({4-[2-(4-ethylpiperazin-1-yl)ethyl]phenyl}amino)-4-fluoro-6-(6-methoxypyrazin-2-yl)-1,2-dihydro-2,7-naphthyridin-1-one;
8-({4-[2-(4-ethylpiperazin-1-yl)ethyl]phenyl}amino)-6-(1-methyl-1H-pyrazol-4-yl)-1,2-dihydro-2,7-naphthyridin-1-one;
8-({4-[2-(4-ethylpiperazin-1-yl)ethyl]phenyl}amino)-4-fluoro-6-(1-methyl-1H-pyrazol-4-yl)-1,2-dihydro-2,7-naphthyridin-1-one;
8-({4-[2-(4-ethylpiperazin-1-yl)ethyl]phenyl}amino)-4-fluoro-6-(6-methylpyridin-3-yl)-1,2-dihydro-2,7-naphthyridin-1-one;
8-({4-[2-(4-ethylpiperazin-1-yl)ethyl]phenyl}amino)-6-[1-(2-methylpropyl)-1H-pyrazol-4-yl]-1,2-dihydro-2,7-naphthyridin-1-one;
8-({4-[2-(4-ethylpiperazin-1-yl)ethyl]phenyl}amino)-4-fluoro-6-[1-(2-methylpropyl)-1H-pyrazol-4-yl]-1,2-dihydro-2,7-naphthyridin-1-one;
8-({4-[2-(4-ethylpiperazin-1-yl)ethyl]phenyl}amino)-6-[(5-fluoropyridin-2-yl)amino]-1,2-dihydro-2,7-naphthyridin-1-one;
8-({4-[2-(4-ethylpiperazin-1-yl)ethyl]phenyl}amino)-6-{[5-(trifluoromethyl)pyridin-2-yl]amino}-1,2-dihydro-2,7-naphthyridin-1-one;
6-{[1-({4-[2-(4-ethylpiperazin-1-yl)ethyl]phenyl}amino)-8-oxo-7,8-dihydro-2,7-naphthyridin-3-yl]amino}pyridine-3-carboxylic acid;
8-({3-methyl-4-[(2R)-2-methylmorpholin-4-yl]phenyl}amino)-6-[(5-methylpyridin-2-yl)amino]-1,2-dihydro-2,7-naphthyridin-1-one;
6-{[1-({3-methyl-4-[(2R)-2-methylmorpholin-4-yl]phenyl}amino)-8-oxo-7,8-dihydro-2,7-naphthyridin-3-yl]amino}pyridine-3-carboxamide;
6-{[1-({3-methyl-4-[(2R)-2-methylmorpholin-4-yl]phenyl}amino)-8-oxo-7,8-dihydro-2,7-naphthyridin-3-yl]amino}pyridine-3-carbonitrile;
8-({3-methyl-4-[(2R)-2-methylmorpholin-4-yl]phenyl}amino)-6-{[5-(trifluoromethyl)pyridin-2-yl]amino}-1,2-dihydro-2,7-naphthyridin-1-one;
8-[(4-{1-[(1-aminocyclopropyl)carbonyl]piperidin-4-yl}-3-methylphenyl)amino]-6-(6-methoxypyrazin-2-yl)-1,2-dihydro-2,7-naphthyridin-1-one;
8-({4-[1-(3,3-dimethylbutyl)piperidin-4-yl]-3-methylphenyl}amino)-6-(6-methoxypyrazin-2-yl)-1,2-dihydro-2,7-naphthyridin-1-one;
8-{[4-(1-cyclopentylpiperidin-4-yl)-3-methylphenyl]amino}-6-(6-methoxypyrazin-2-yl)-1,2-dihydro-2,7-naphthyridin-1-one;
8-[(4-{1-[2-(dimethylamino)ethyl]piperidin-4-yl}-3-methylphenyl)amino]-6-(6-methoxypyrazin-2-yl)-1,2-dihydro-2,7-naphthyridin-1-one;
8-({4-[(4-ethylpiperazin-1-yl)carbonyl]phenyl}amino)-6-(6-methoxypyrazin-2-yl)-1,2-dihydro-2,7-naphthyridin-1-one;
3-[3-(4-{[3-(6-methoxypyrazin-2-yl)-8-oxo-7,8-dihydro-2,7-naphthyridin-1-yl]amino}phenyl)pyrrolidin-1-yl]propanenitrile;
3-{4-[2-(4-{[3-(6-methoxypyrazin-2-yl)-8-oxo-7,8-dihydro-2,7-naphthyridin-1-yl]amino}phenyl)ethyl]piperazin-1-yl}propanenitrile;
3-[4-(4-{[3-(2-aminopyrimidin-4-yl)-8-oxo-7,8-dihydro-2,7-naphthyridin-1-yl]amino}-2-methylphenyl)piperidin-1-yl]propanenitrile;
3-{4-[4-({3-[6-(cyclopropylamino)pyrazin-2-yl]-8-oxo-7,8-dihydro-2,7-naphthyridin-1-yl}amino)-2-methylphenyl]piperidin-1-yl}propanenitrile;
6-(6-methoxypyrazin-2-yl)-8-({3-methyl-4-[1-(oxetan-3-yl)piperidin-4-yl]phenyl}amino)-1,2-dihydro-2,7-naphthyridin-1-one;
3-[4-(4-{[3-(6-amino-5-methylpyridin-3-yl)-8-oxo-7,8-dihydro-2,7-naphthyridin-1-yl]amino}-2-methylphenyl)piperidin-1-yl]propanenitrile;
6-(6-methoxypyrazin-2-yl)-8-({3-methyl-4-[1-(propan-2-yl)piperidin-4-yl]phenyl}amino)-1,2-dihydro-2,7-naphthyridin-1-one;
3-[4-(4-{[3-(2-amino-6-methylpyrimidin-4-yl)-8-oxo-7,8-dihydro-2,7-naphthyridin-1-yl]amino}-2-methylphenyl)piperidin-1-yl]propanenitrile;
8-({4-[1-(2,3-dihydroxypropyl)piperidin-4-yl]-3-methylphenyl}amino)-6-(6-methoxypyrazin-2-yl)-1,2-dihydro-2,7-naphthyridin-1-one;
2-[4-(4-{[3-(6-methoxypyrazin-2-yl)-8-oxo-7,8-dihydro-2,7-naphthyridin-1-yl]amino}-2-methylphenyl)piperidin-1-yl]-N,N-dimethylacetamide;
6-(5-amino-6-methoxypyrazin-2-yl)-8-{[3-methyl-4-(piperidin-4-yl)phenyl]amino}-1,2-dihydro-2,7-naphthyridin-1-one;
3-[4-(4-{[3-(5-amino-6-methoxypyrazin-2-yl)-8-oxo-7,8-dihydro-2,7-naphthyridin-1-yl]amino}-2-methylphenyl)piperidin-1-yl]propanenitrile;
6-[(1-{[4-(1-cyclopropylpiperidin-4-yl)-3-methylphenyl]amino}-8-oxo-7,8-dihydro-2,7-naphthyridin-3-yl)amino]pyridine-3-carboxamide;
8-{[4-(1-cyclopropylpiperidin-4-yl)-3-methylphenyl]amino}-6-(3,4-dimethoxyphenyl)-1,2-dihydro-2,7-naphthyridin-1-one;
8-({3-[4-(2-methoxyethyl)piperazin-1-yl]-1H-indazol-6-yl}amino)-6-(pyrimidin-5-yl)-1,2-dihydro-2,7-naphthyridin-1-one;
8-({3-[4-(2-methoxyethyl)piperazin-1-yl]-1H-indazol-6-yl}amino)-6-(6-methylpyridin-3-yl)-1,2-dihydro-2,7-naphthyridin-1-one;
8-({3-[4-(2-methoxyethyl)piperazin-1-yl]-1H-indazol-6-yl}amino)-6-(2-methoxypyrimidin-5-yl)-1,2-dihydro-2,7-naphthyridin-1-one;
8-{[4-(1-cyclopropylpiperidin-4-yl)-3-methylphenyl]amino}-6-[(4-methylpyridin-2-yl)amino]-1,2-dihydro-2,7-naphthyridin-1-one;
8-{[4-(1-cyclopropylpiperidin-4-yl)-3-methylphenyl]amino}-6-[(4-ethylpyridin-2-yl)amino]-1,2-dihydro-2,7-naphthyridin-1-one;
8-{[4-(1-cyclopropylpiperidin-4-yl)-3-methylphenyl]amino}-6-[(4-propylpyridin-2-yl)amino]-1,2-dihydro-2,7-naphthyridin-1-one;
8-{[4-(1-cyclopropylpiperidin-4-yl)-3-methylphenyl]amino}-6-{[4-(trifluoromethyl)pyridin-2-yl]amino}-1,2-dihydro-2,7-naphthyridin-1-one;
8-{[4-(1-cyclopropylpiperidin-4-yl)-3-methylphenyl]amino}-6-[(4,6-dimethylpyridin-2-yl)amino]-1,2-dihydro-2,7-naphthyridin-1-one;
6-[(1-{[4-(1-cyclopropylpiperidin-4-yl)-3-methylphenyl]amino}-8-oxo-7,8-dihydro-2,7-naphthyridin-3-yl)amino]pyridine-3-sulfonamide;
8-({3-methyl-4-[(2R)-2-methylmorpholin-4-yl]phenyl}amino)-6-[(4-methylpyridin-2-yl)amino]-1,2-dihydro-2,7-naphthyridin-1-one;
8-({4-[2-(4-ethylpiperazin-1-yl)ethyl]phenyl}amino)-6-[(4-methylpyridin-2-yl)amino]-1,2-dihydro-2,7-naphthyridin-1-one;
8-({4-[2-(4-ethylpiperazin-1-yl)ethyl]phenyl}amino)-6-[(4-ethylpyridin-2-yl)amino]-1,2-dihydro-2,7-naphthyridin-1-one;
8-({4-[2-(4-ethylpiperazin-1-yl)ethyl]phenyl}amino)-6-{[4-(trifluoromethyl)pyridin-2-yl]amino}-1,2-dihydro-2,7-naphthyridin-1-one;
2-{[1-({4-[2-(4-ethylpiperazin-1-yl)ethyl]phenyl}amino)-8-oxo-7,8-dihydro-2,7-naphthyridin-3-yl]amino}pyridine-4-carboxylic acid;
6-[(4,6-dimethylpyridin-2-yl)amino]-8-({4-[2-(4-ethylpiperazin-1-yl)ethyl]phenyl}amino)-1,2-dihydro-2,7-naphthyridin-1-one;
6-[(4-methylpyridin-2-yl)amino]-8-{[4-(morpholin-4-yl)phenyl]amino}-1,2-dihydro-2,7-naphthyridin-1-one;
6-(2-methylpyrimidin-5-yl)-8-({4-[4-(morpholin-4-yl)cyclohexyl]phenyl}amino)-1,2-dihydro-2,7-naphthyridin-1-one;
8-({4-[1-(2-methoxyethyl)piperidin-4-yl]-3-methylphenyl}amino)-6-(2-methylpyrimidin-5-yl)-1,2-dihydro-2,7-naphthyridin-1-one;
8-({4-[1-(3-fluoropropyl)piperidin-4-yl]-3-methylphenyl}amino)-6-(2-methylpyrimidin-5-yl)-1,2-dihydro-2,7-naphthyridin-1-one;
3-[4-(2-methyl-4-{[3-(2-methylpyrimidin-5-yl)-8-oxo-7,8-dihydro-2,7-naphthyridin-1-yl]amino}-phenyl)piperidin-1-yl]propanenitrile;
N-(2-methoxyethyl)-4-{[3-(2-methylpyrimidin-5-yl)-8-oxo-7,8-dihydro-2,7-naphthyridin-1-yl]amino}benzamide;
6-(6-methoxypyrazin-2-yl)-8-[(3-methyl-4-{1-[(3-methyloxetan-3-yl)carbonyl]piperidin-4-yl}phenyl)amino]-1,2-dihydro-2,7-naphthyridin-1-one;
6-(6-methoxypyrazin-2-yl)-8-({3-methyl-4-[1-(2-methylbutanoyl)piperidin-4-yl]phenyl}amino)-1,2-dihydro-2,7-naphthyridin-1-one;
6-(6-methoxypyrazin-2-yl)-8-({3-methyl-4-[1-(oxolan-2-ylmethyl)piperidin-4-yl]phenyl}amino)-1,2-dihydro-2,7-naphthyridin-1-one;
8-[(4-{1-[(2,2-difluorocyclopropyl)methyl]piperidin-4-yl}-3-methylphenyl)amino]-6-(6-methoxypyrazin-2-yl)-1,2-dihydro-2,7-naphthyridin-1-one;
8-({4-[1-(2-fluoroethyl)piperidin-4-yl]-3-methylphenyl}amino)-6-(6-methoxypyrazin-2-yl)-1,2-dihydro-2,7-naphthyridin-1-one;
6-(6-methoxypyrazin-2-yl)-8-{[3-methyl-4-(1-propanoylpiperidin-4-yl)phenyl]amino}-1,2-dihydro-2,7-naphthyridin-1-one;
8-({4-[1-(cyclopropylmethyl)piperidin-4-yl]-3-methylphenyl}amino)-6-(6-methoxypyrazin-2-yl)-1,2-dihydro-2,7-naphthyridin-1-one;
4-{[3-(6-methoxypyrazin-2-yl)-8-oxo-7,8-dihydro-2,7-naphthyridin-1-yl]amino}-N-[2-(pyrrolidin-1-yl)ethyl]benzamide;
N-{[(2R)-1-ethylpyrrolidin-2-yl]methyl}-4-{[3-(6-methoxypyrazin-2-yl)-8-oxo-7,8-dihydro-2,7-naphthyridin-1-yl]amino}benzamide;
6-(6-methoxypyrazin-2-yl)-8-[(3-methyl-4-{1-[2-methyl-2-(methylamino)propanoyl]piperidin-4-yl}phenyl)amino]-1,2-dihydro-2,7-naphthyridin-1-one;
8-({4-[1-(2-ethoxyethyl)piperidin-4-yl]-3-methylphenyl}amino)-6-(6-methoxypyrazin-2-yl)-1,2-dihydro-2,7-naphthyridin-1-one;
2-[4-(4-{[3-(6-methoxypyrazin-2-yl)-8-oxo-7,8-dihydro-2,7-naphthyridin-1-yl]amino}-2-methylphenyl)piperidin-1-yl]-N,N-dimethylpropanamide;
3-[4-(4-{[3-(6-methoxypyrazin-2-yl)-8-oxo-7,8-dihydro-2,7-naphthyridin-1-yl]amino}-2-methylphenyl)piperidin-1-yl]butanenitrile;
4-[4-(4-{[3-(6-methoxypyrazin-2-yl)-8-oxo-7,8-dihydro-2,7-naphthyridin-1-yl]amino}-2-methylphenyl)piperidin-1-yl]butanenitrile;
2-[4-(4-{[3-(6-methoxypyrazin-2-yl)-8-oxo-7,8-dihydro-2,7-naphthyridin-1-yl]amino}-2-methylphenyl)piperidin-1-yl]-N-methylpropanamide;
6-[(4-ethylpyridin-2-yl)amino]-8-(propan-2-ylamino)-1,2-dihydro-2,7-naphthyridin-1-one;
2-{[8-oxo-1-(propan-2-ylamino)-7,8-dihydro-2,7-naphthyridin-3-yl]amino}pyridine-4-carboxylic acid;
6-[(4-methylpyridin-2-yl)amino]-8-(propan-2-ylamino)-1,2-dihydro-2,7-naphthyridin-1-one;
6-[(4-chloropyridin-2-yl)amino]-8-(propan-2-ylamino)-1,2-dihydro-2,7-naphthyridin-1-one;
6-[(4-chloropyridin-2-yl)amino]-8-{[4-(1-cyclopropylpiperidin-4-yl)-3-methylphenyl]amino}-1,2-dihydro-2,7-naphthyridin-1-one;
8-{[4-(1-cyclopropylpiperidin-4-yl)-3-methylphenyl]amino}-6-[6-(dimethylamino)pyridin-3-yl]-1,2-dihydro-2,7-naphthyridin-1-one;
6-(3,6-dihydro-2H-pyran-4-yl)-8-{[4-(morpholin-4-yl)phenyl]amino}-1,2-dihydro-2,7-naphthyridin-1-one;
8-{[4-(morpholin-4-yl)phenyl]amino}-6-(oxan-4-yl)-1,2-dihydro-2,7-naphthyridin-1-one;
8-{[4-(morpholin-4-yl)phenyl]amino}-6-(1,2,3,6-tetrahydropyridin-4-yl)-1,2-dihydro-2,7-naphthyridin-1-one;
6-(6-methoxypyrazin-2-yl)-8-[(3-methyl-4-{1-[(oxolan-3-yl)carbonyl]piperidin-4-yl}phenyl)amino]-1,2-dihydro-2,7-naphthyridin-1-one;
8-({4-[1-(2-chloro-3-methoxypropanoyl)piperidin-4-yl]-3-methylphenyl}amino)-6-(6-methoxypyrazin-2-yl)-1,2-dihydro-2,7-naphthyridin-1-one;
8-({4-[1-(1-methoxypropan-2-yl)piperidin-4-yl]-3-methylphenyl}amino)-6-(6-methoxypyrazin-2-yl)-1,2-dihydro-2,7-naphthyridin-1-one;
2-[4-(4-{[3-(6-methoxypyrazin-2-yl)-8-oxo-7,8-dihydro-2,7-naphthyridin-1-yl]amino}-2-methylphenyl)piperidin-1-yl]-N-(propan-2-yl)acetamide;
N-methoxy-2-[4-(4-{[3-(6-methoxypyrazin-2-yl)-8-oxo-7,8-dihydro-2,7-naphthyridin-1-yl]amino}-2-methylphenyl)piperidin-1-yl]-N-methylacetamide;
4-(4-{[3-(6-methoxypyrazin-2-yl)-8-oxo-7,8-dihydro-2,7-naphthyridin-1-yl]amino}-2-methylphenyl)-N,N-dimethylpiperidine-1-carboxamide;
3-[4-(4-{[3-(6-methoxypyrazin-2-yl)-8-oxo-7,8-dihydro-2,7-naphthyridin-1-yl]amino}-2-methylphenyl)piperidin-1-yl]-2-methylpropanenitrile;
N-ethyl-2-[4-(4-{[3-(6-methoxypyrazin-2-yl)-8-oxo-7,8-dihydro-2,7-naphthyridin-1-yl]amino}-2-methylphenyl)piperidin-1-yl]acetamide;
N,N-diethyl-2-[4-(4-{[3-(6-methoxypyrazin-2-yl)-8-oxo-7,8-dihydro-2,7-naphthyridin-1-yl]amino}-2-methylphenyl)piperidin-1-yl]acetamide;
6-(6-methoxypyrazin-2-yl)-8-[(3-methyl-4-{1-[(pyrrolidin-1-yl)carbonyl]piperidin-4-yl}phenyl)amino]-1,2-dihydro-2,7-naphthyridin-1-one;
6-(6-methoxypyrazin-2-yl)-8-[(3-methyl-4-{1-[(2-oxoimidazolidin-1-yl)carbonyl]piperidin-4-yl}phenyl)amino]-1,2-dihydro-2,7-naphthyridin-1-one;
6-(6-methoxypyrazin-2-yl)-8-[(3-methyl-4-{1-[(morpholin-4-yl)carbonyl]piperidin-4-yl}phenyl)amino]-1,2-dihydro-2,7-naphthyridin-1-one;
methyl 2-[4-(4-{[3-(6-methoxypyrazin-2-yl)-8-oxo-7,8-dihydro-2,7-naphthyridin-1-yl]amino}-2-methylphenyl)piperidin-1-yl]propanoate;
8-({3-[4-(2-methoxyethyl)piperazin-1-yl]-1H-indazol-6-yl}amino)-6-(6-methoxypyrazin-2-yl)-1,2-dihydro-2,7-naphthyridin-1-one;
8-{[4-(1-cyclopropylpiperidin-4-yl)-3-methylphenyl]amino}-6-[(4-methoxypyridin-2-yl)amino]-1,2-dihydro-2,7-naphthyridin-1-one;
8-{[4-(1-cyclopropylpiperidin-4-yl)-3-methylphenyl]amino}-6-{[4-(hydroxymethyl)pyridin-2-yl]amino}-1,2-dihydro-2,7-naphthyridin-1-one;
8-{[4-(morpholin-4-yl)phenyl]amino}-6-(piperidin-4-yl)-1,2-dihydro-2,7-naphthyridin-1-one;
6-[1-(2-methoxyethyl)piperidin-4-yl]-8-{[4-(morpholin-4-yl)phenyl]amino}-1,2-dihydro-2,7-naphthyridin-1-one;
8-{[4-(morpholin-4-yl)phenyl]amino}-6-[1-(propan-2-yl)piperidin-4-yl]-1,2-dihydro-2,7-naphthyridin-1-one;
6-(2-aminopyrimidin-5-yl)-2-[(2,2-difluorocyclopropyl)methyl]-8-{[4-(morpholin-4-yl)phenyl]amino}-1,2-dihydro-2,7-naphthyridin-1-one;
6-acetyl-8-({3-methyl-4-[(2R)-2-methylmorpholin-4-yl]phenyl}amino)-1,2-dihydro-2,7-naphthyridin-1-one;
8-[(4-{2,6-diazaspiro[3.3]heptan-2-yl}phenyl)amino]-6-(6-methoxypyrazin-2-yl)-1,2-dihydro-2,7-naphthyridin-1-one;
2-[(1-{[4-(1-cyclopropylpiperidin-4-yl)-3-methylphenyl]amino}-8-oxo-7,8-dihydro-2,7-naphthyridin-3-yl)amino]pyridine-4-carboxylic acid;
8-({4-[1-(2-hydroxy-3-methylbutanoyl)piperidin-4-yl]-3-methylphenyl}amino)-6-(6-methoxypyrazin-2-yl)-1,2-dihydro-2,7-naphthyridin-1-one;
6-(6-methoxypyrazin-2-yl)-8-{[4-(2-{octahydropyrrolo[1,2-a]piperazin-2-yl}ethyl)phenyl]amino}-1,2-dihydro-2,7-naphthyridin-1-one;
6-acetyl-8-{[4-(1-cyclopropylpiperidin-4-yl)-3-methylphenyl]amino}-1,2-dihydro-2,7-naphthyridin-1-one;
8-({4-[1-(3-methoxy-2,2-dimethylpropanoyl)piperidin-4-yl]-3-methylphenyl}amino)-6-(6-methoxypyrazin-2-yl)-1,2-dihydro-2,7-naphthyridin-1-one;
8-{[4-(1-cyclopropylpiperidin-4-yl)-3-methylphenyl]amino}-6-[(4,6-dimethylpyridin-2-yl)amino]-1,2-dihydro-2,7-naphthyridin-1-one;
6-[(4-methylpyridin-2-yl)amino]-8-(propan-2-ylamino)-1,2-dihydro-2,7-naphthyridin-1-one;
8-({4-[4-(morpholin-4-yl)cyclohexyl]phenyl}amino)-6-(pyrimidin-5-yl)-1,2-dihydro-2,7-naphthyridin-1-one;
6-(2-methylpyrimidin-5-yl)-8-({4-[4-(morpholin-4-yl)cyclohexyl]phenyl}amino)-1,2-dihydro-2,7-naphthyridin-1-one;
6-(6-methoxypyrazin-2-yl)-8-({4-[4-(morpholin-4-yl)cyclohexyl]phenyl}amino)-1,2-dihydro-2,7-naphthyridin-1-one;
6-(2-ethoxyethyl)-8-({3-methyl-4-[(2R)-2-methylmorpholin-4-yl]phenyl}amino)-1,2-dihydro-2,7-naphthyridin-1-one;
8-{[4-(3,6-dihydro-2H-thiopyran-4-yl)-3-methylphenyl]amino}-6-(6-methoxypyrazin-2-yl)-1,2-dihydro-2,7-naphthyridin-1-one;
6-(6-methoxypyrazin-2-yl)-8-{[3-methyl-4-(1,2,3,6-tetrahydropyridin-4-yl)phenyl]amino}-1,2-dihydro-2,7-naphthyridin-1-one;
8-{[4-(1-cyclopropylpiperidin-4-yl)-3-methylphenyl]amino}-6-[(Z)-2-ethoxyethenyl]-1,2-dihydro-2,7-naphthyridin-1-one;
6-(3,6-dihydro-2H-thiopyran-4-yl)-8-{[4-(morpholin-4-yl)phenyl]amino}-1,2-dihydro-2,7-naphthyridin-1-one;
8-({3-methyl-4-[(2R)-2-methylmorpholin-4-yl]phenyl}amino)-6-propyl-1,2-dihydro-2,7-naphthyridin-1-one;
8-{[4-(morpholin-4-yl)phenyl]amino}-6-(thian-4-yl)-1,2-dihydro-2,7-naphthyridin-1-one;
8-{[4-(1-cyclopropylpiperidin-4-yl)-3-methylphenyl]amino}-6-(2-ethoxyethyl)-1,2-dihydro-2,7-naphthyridin-1-one;
8-{[4-(1-cyclopropylpiperidin-4-yl)-3-methylphenyl]amino}-6-[(1E)-prop-1-en-1-yl]-1,2-dihydro-2,7-naphthyridin-1-one;
8-{[4-(1-cyclopropylpiperidin-4-yl)-3-methylphenyl]amino}-6-(prop-1-en-2-yl)-1,2-dihydro-2,7-naphthyridin-1-one;
2-[4-(4-{[3-(6-methoxypyrazin-2-yl)-8-oxo-7,8-dihydro-2,7-naphthyridin-1-yl]amino}-2-methylphenyl)piperidin-1-yl]propanoic acid;
methyl 2-[4-(4-{[3-(6-methoxypyrazin-2-yl)-8-oxo-7,8-dihydro-2,7-naphthyridin-1-yl]amino}-2-methylphenyl)piperidin-1-yl]butanoate;
2-[4-(4-{[3-(6-methoxypyrazin-2-yl)-8-oxo-7,8-dihydro-2,7-naphthyridin-1-yl]amino}-2-methylphenyl)piperidin-1-yl]butanoic acid;
N-ethyl-2-[4-(4-{[3-(6-methoxypyrazin-2-yl)-8-oxo-7,8-dihydro-2,7-naphthyridin-1-yl]amino}-2-methylphenyl)piperidin-1-yl]propanamide;
N-cyclopropyl-2-[4-(4-{[3-(6-methoxypyrazin-2-yl)-8-oxo-7,8-dihydro-2,7-naphthyridin-1-yl]amino}-2-methylphenyl)piperidin-1-yl]propanamide;
N-(2-hydroxyethyl)-2-[4-(4-{[3-(6-methoxypyrazin-2-yl)-8-oxo-7,8-dihydro-2,7-naphthyridin-1-yl]amino}-2-methylphenyl)piperidin-1-yl]propanamide;
N-(2-methoxyethyl)-2-[4-(4-{[3-(6-methoxypyrazin-2-yl)-8-oxo-7,8-dihydro-2,7-naphthyridin-1-yl]amino}-2-methylphenyl)piperidin-1-yl]propanamide;
2-[4-(4-{[3-(6-methoxypyrazin-2-yl)-8-oxo-7,8-dihydro-2,7-naphthyridin-1-yl]amino}-2-methylphenyl)piperidin-1-yl]-N-methylbutanamide;
2-methyl-6,8-bis[(4-methylpyridin-2-yl)amino]-1,2-dihydro-2,7-naphthyridin-1-one;
8-(cyclopropylamino)-6-(pyridin-2-ylamino)-1,2-dihydro-2,7-naphthyridin-1-one;
8-(cyclopropylamino)-6-[(4-methylpyridin-2-yl)amino]-1,2-dihydro-2,7-naphthyridin-1-one;
6-[(4-chloropyridin-2-yl)amino]-8-(cyclopropylamino)-1,2-dihydro-2,7-naphthyridin-1-one;
8-(cyclopropylamino)-6-[(4-methoxypyridin-2-yl)amino]-1,2-dihydro-2,7-naphthyridin-1-one;
2-methyl-6-[(4-methylpyridin-2-yl)amino]-8-(propan-2-ylamino)-1,2-dihydro-2,7-naphthyridin-1-one;
8-{[4-(1-{[1-(methoxymethyl)cyclopropyl]carbonyl}piperidin-4-yl)-3-methylphenyl]amino}-6-(6-methoxypyrazin-2-yl)-1,2-dihydro-2,7-naphthyridin-1-one;
8-({6-[(2R,6S)-2,6-dimethylmorpholin-4-yl]pyridin-3-yl}amino)-6-(2-methylpyrimidin-5-yl)-1,2-dihydro-2,7-naphthyridin-1-one;
8-({6-[(2R,6S)-2,6-dimethylmorpholin-4-yl]pyridin-3-yl}amino)-6-(pyrimidin-5-yl)-1,2-dihydro-2,7-naphthyridin-1-one;
6-(5-amino-6-methylpyrazin-2-yl)-8-({6-[(2R,6S)-2,6-dimethylmorpholin-4-yl]pyridin-3-yl}amino)-1,2-dihydro-2,7-naphthyridin-1-one;
8-{[4-(4-methoxypiperidin-1-yl)phenyl]amino}-6-(6-methoxypyrazin-2-yl)-1,2-dihydro-2,7-naphthyridin-1-one;
8-({3-methyl-4-[(2R)-2-methylmorpholin-4-yl]phenyl}amino)-6-(propan-2-yl)-1,2-dihydro-2,7-naphthyridin-1-one;
8-{[4-(1-cyclopropylpiperidin-4-yl)-3-methylphenyl]amino}-6-propyl-1,2-dihydro-2,7-naphthyridin-1-one;
8-{[4-(1-cyclopropylpiperidin-4-yl)-3-methylphenyl]amino}-6-(propan-2-yl)-1,2-dihydro-2,7-naphthyridin-1-one;
6-acetyl-8-{[3-methyl-4-(piperidin-4-yl)phenyl]amino}-1,2-dihydro-2,7-naphthyridin-1-one;
6-[(1E)-1-(methoxyimino)ethyl]-8-({3-methyl-4-[(2R)-2-methylmorpholin-4-yl]phenyl}amino)-1,2-dihydro-2,7-naphthyridin-1-one;
8-(butan-2-ylamino)-6-[(4-methylpyridin-2-yl)amino]-1,2-dihydro-2,7-naphthyridin-1-one;
8-(butan-2-ylamino)-6-(pyridin-2-ylamino)-1,2-dihydro-2,7-naphthyridin-1-one;
8-(butan-2-ylamino)-6-[(5-fluoropyridin-2-yl)amino]-1,2-dihydro-2,7-naphthyridin-1-one;
8-[(1-hydroxypropan-2-yl)amino]-6-[(4-methylpyridin-2-yl)amino]-1,2-dihydro-2,7-naphthyridin-1-one;
8-[(1-methoxypropan-2-yl)amino]-6-[(4-methylpyridin-2-yl)amino]-1,2-dihydro-2,7-naphthyridin-1-one;
8-{[(1R,2R)-2-hydroxycyclopentyl]amino}-6-[(4-methylpyridin-2-yl)amino]-1,2-dihydro-2,7-naphthyridin-1-one;
8-[(3-methylbutan-2-yl)amino]-6-[(4-methylpyridin-2-yl)amino]-1,2-dihydro-2,7-naphthyridin-1-one;
6-(1-ethoxyethyl)-8-({3-methyl-4-[(2R)-2-methylmorpholin-4-yl]phenyl}amino)-1,2-dihydro-2,7-naphthyridin-1-one;
6-[(1E)-1-(methoxyimino)ethyl]-8-{[3-methyl-4-(piperidin-4-yl)phenyl]amino}-1,2-dihydro-2,7-naphthyridin-1-one;
8-(cyclopropylamino)-6-{[4-(hydroxymethyl)pyridin-2-yl]amino}-1,2-dihydro-2,7-naphthyridin-1-one;
8-(cyclopropylamino)-6-[(4-ethylpyridin-2-yl)amino]-1,2-dihydro-2,7-naphthyridin-1-one;
8-(cyclopropylamino)-6-{[4-(trifluoromethyl)pyridin-2-yl]amino}-1,2-dihydro-2,7-naphthyridin-1-one;
2-{[1-(cyclopropylamino)-8-oxo-7,8-dihydro-2,7-naphthyridin-3-yl]amino}pyridine-4-carboxamide;
8-(cyclopropylamino)-2-methyl-6-[(4-methylpyridin-2-yl)amino]-1,2-dihydro-2,7-naphthyridin-1-one;
8-(ethylamino)-6-[(4-methylpyridin-2-yl)amino]-1,2-dihydro-2,7-naphthyridin-1-one;
8-[(2-hydroxyethyl)amino]-6-[(4-methylpyridin-2-yl)amino]-1,2-dihydro-2,7-naphthyridin-1-one;
8-[(2-methoxyethyl)amino]-6-[(4-methylpyridin-2-yl)amino]-1,2-dihydro-2,7-naphthyridin-1-one;
8-{[2-(dimethylamino)ethyl]amino}-6-[(4-methylpyridin-2-yl)amino]-1,2-dihydro-2,7-naphthyridin-1-one;
8-({[(2R)-1-ethylpyrrolidin-2-yl]methyl}amino)-6-[(4-methylpyridin-2-yl)amino]-1,2-dihydro-2,7-naphthyridin-1-one;
8-[(1-methylpiperidin-4-yl)amino]-6-[(4-methylpyridin-2-yl)amino]-1,2-dihydro-2,7-naphthyridin-1-one;
6-[(4-methylpyridin-2-yl)amino]-8-({[4-(morpholin-4-yl)phenyl]methyl}amino)-1,2-dihydro-2,7-naphthyridin-1-one;
6-[(4-methylpyridin-2-yl)amino]-8-{[(2R)-oxolan-2-ylmethyl]amino}-1,2-dihydro-2,7-naphthyridin-1-one;
8-(butylamino)-6-[(4-methylpyridin-2-yl)amino]-1,2-dihydro-2,7-naphthyridin-1-one;
8-[(4-methylpentan-2-yl)amino]-6-[(4-methylpyridin-2-yl)amino]-1,2-dihydro-2,7-naphthyridin-1-one;
6-[(4-methylpyridin-2-yl)amino]-8-(pyrrolidin-1-yl)-1,2-dihydro-2,7-naphthyridin-1-one;
8-{[(1S,2R)-2-hydroxycyclopentyl]amino}-6-[(4-methylpyridin-2-yl)amino]-1,2-dihydro-2,7-naphthyridin-1-one;
6-[(4-methylpyridin-2-yl)amino]-8-(morpholin-4-yl)-1,2-dihydro-2,7-naphthyridin-1-one;
6-[(4-methylpyridin-2-yl)amino]-8-(oxolan-3-ylamino)-1,2-dihydro-2,7-naphthyridin-1-one;
8-[(1-hydroxypropan-2-yl)amino]-6-(pyridin-2-ylamino)-1,2-dihydro-2,7-naphthyridin-1-one;
8-[(4-methylpentan-2-yl)amino]-6-(pyridin-2-ylamino)-1,2-dihydro-2,7-naphthyridin-1-one;
8-[(1-methoxypropan-2-yl)amino]-6-(pyridin-2-ylamino)-1,2-dihydro-2,7-naphthyridin-1-one;
8-{[(1R,2R)-2-hydroxycyclopentyl]amino}-6-(pyridin-2-ylamino)-1,2-dihydro-2,7-naphthyridin-1-one;
8-[(3-methylbutan-2-yl)amino]-6-(pyridin-2-ylamino)-1,2-dihydro-2,7-naphthyridin-1-one;
6-(pyridin-2-ylamino)-8-(pyrrolidin-1-yl)-1,2-dihydro-2,7-naphthyridin-1-one;
8-{[(1S,2R)-2-hydroxycyclopentyl]amino}-6-(pyridin-2-ylamino)-1,2-dihydro-2,7-naphthyridin-1-one;
8-(morpholin-4-yl)-6-(pyridin-2-ylamino)-1,2-dihydro-2,7-naphthyridin-1-one;
8-(oxolan-3-ylamino)-6-(pyridin-2-ylamino)-1,2-dihydro-2,7-naphthyridin-1-one;
6-(2-aminopyrimidin-5-yl)-8-[(1-methoxypropan-2-yl)amino]-1,2-dihydro-2,7-naphthyridin-1-one;
6-(2-aminopyrimidin-5-yl)-8-(pyrrolidin-1-yl)-1,2-dihydro-2,7-naphthyridin-1-one;
6-(2-aminopyrimidin-5-yl)-8-{[(1S,2R)-2-hydroxycyclopentyl]amino}-1,2-dihydro-2,7-naphthyridin-1-one;
8-(propan-2-ylamino)-6-[(4-propylpyridin-2-yl)amino]-1,2-dihydro-2,7-naphthyridin-1-one;
8-[(1-benzylpyrrolidin-3-yl)amino]-6-[(4-methylpyridin-2-yl)amino]-1,2-dihydro-2,7-naphthyridin-1-one;
6-[(4-methylpyridin-2-yl)amino]-8-{[(2S)-oxolan-2-ylmethyl]amino}-1,2-dihydro-2,7-naphthyridin-1-one;
8-(cyclobutylamino)-6-[(4-methylpyridin-2-yl)amino]-1,2-dihydro-2,7-naphthyridin-1-one;
8-{[2-(3-chlorophenyl)-2-hydroxyethyl]amino}-6-[(4-methylpyridin-2-yl)amino]-1,2-dihydro-2,7-naphthyridin-1-one;
8-{[(2R)-2-hydroxypropyl]amino}-6-[(4-methylpyridin-2-yl)amino]-1,2-dihydro-2,7-naphthyridin-1-one;
8-{[(2S)-2-hydroxypropyl]amino}-6-[(4-methylpyridin-2-yl)amino]-1,2-dihydro-2,7-naphthyridin-1-one;
6-(2-aminopyrimidin-5-yl)-8-[(2-hydroxyethyl)amino]-1,2-dihydro-2,7-naphthyridin-1-one;
6-(2-aminopyrimidin-5-yl)-8-[(2-methoxyethyl)amino]-1,2-dihydro-2,7-naphthyridin-1-one;
8-[(3-hydroxypropyl)amino]-6-[(4-methylpyridin-2-yl)amino]-1,2-dihydro-2,7-naphthyridin-1-one;
6-[(4-methylpyridin-2-yl)amino]-8-[(piperidin-4-ylmethyl)amino]-1,2-dihydro-2,7-naphthyridin-1-one;
8-({[(2S)-1-ethylpyrrolidin-2-yl]methyl}amino)-6-[(4-methylpyridin-2-yl)amino]-1,2-dihydro-2,7-naphthyridin-1-one;
8-(benzylamino)-6-[(4-methylpyridin-2-yl)amino]-1,2-dihydro-2,7-naphthyridin-1-one;
6-[(4-methylpyridin-2-yl)amino]-8-[(2,2,2-trifluoroethyl)amino]-1,2-dihydro-2,7-naphthyridin-1-one;
6-(2-aminopyrimidin-5-yl)-8-[(1-hydroxypropan-2-yl)amino]-1,2-dihydro-2,7-naphthyridin-1-one;
8-(propan-2-ylamino)-6-(pyrazin-2-ylamino)-1,2-dihydro-2,7-naphthyridin-1-one;
6-(2-aminopyrimidin-5-yl)-8-{[(1R,2R)-2-hydroxycyclopentyl]amino}-1,2-dihydro-2,7-naphthyridin-1-one;
6-[(5-chloropyridin-2-yl)amino]-8-(propan-2-ylamino)-1,2-dihydro-2,7-naphthyridin-1-one;
8-{[4-(4-oxocyclohexyl)phenyl]amino}-6-(pyrimidin-5-yl)-1,2-dihydro-2,7-naphthyridin-1-one;
6-{[8-oxo-1-(propan-2-ylamino)-7,8-dihydro-2,7-naphthyridin-3-yl]amino}pyridine-3-carbonitrile;
6-(2-aminopyrimidin-5-yl)-8-(oxolan-3-ylamino)-1,2-dihydro-2,7-naphthyridin-1-one;
6-{[8-oxo-1-(propan-2-ylamino)-7,8-dihydro-2,7-naphthyridin-3-yl]amino}pyridine-3-carboxamide;
8-[(3-methoxypropyl)amino]-6-[(4-methylpyridin-2-yl)amino]-1,2-dihydro-2,7-naphthyridin-1-one;
8-[(2-methylpropyl)amino]-6-[(4-methylpyridin-2-yl)amino]-1,2-dihydro-2,7-naphthyridin-1-one;
8-({4-[4-(dimethylamino)cyclohexyl]phenyl}amino)-6-(pyrimidin-5-yl)-1,2-dihydro-2,7-naphthyridin-1-one;
8-[(2S)-butan-2-ylamino]-6-[(4-methylpyridin-2-yl)amino]-1,2-dihydro-2,7-naphthyridin-1-one;
6-[(4-methylpyridin-2-yl)amino]-8-{[2-(pyridin-3-yl)ethyl]amino}-1,2-dihydro-2,7-naphthyridin-1-one;
8-[(cyclopropylmethyl)amino]-6-[(4-methylpyridin-2-yl)amino]-1,2-dihydro-2,7-naphthyridin-1-one;
6-(2-aminopyrimidin-5-yl)-8-[(2-methylpropyl)amino]-1,2-dihydro-2,7-naphthyridin-1-one;
8-[(4-{4-[(2R)-2-methylmorpholin-4-yl]cyclohexyl}phenyl)amino]-6-(pyrimidin-5-yl)-1,2-dihydro-2,7-naphthyridin-1-one;
6-(2-aminopyrimidin-5-yl)-8-[(2S)-butan-2-ylamino]-1,2-dihydro-2,7-naphthyridin-1-one;
6-(2-aminopyrimidin-5-yl)-8-{[2-(pyridin-3-yl)ethyl]amino}-1,2-dihydro-2,7-naphthyridin-1-one;
8-{[4-(1-tert-butyl-1,2,3,6-tetrahydropyridin-4-yl)phenyl]amino}-6-(pyrimidin-5-yl)-1,2-dihydro-2,7-naphthyridin-1-one;
6-(2-aminopyrimidin-5-yl)-8-[(cyclopropylmethyl)amino]-1,2-dihydro-2,7-naphthyridin-1-one;
8-[(3-methoxypropyl)amino]-6-(pyridin-2-ylamino)-1,2-dihydro-2,7-naphthyridin-1-one;
8-[(2-methylpropyl)amino]-6-(pyridin-2-ylamino)-1,2-dihydro-2,7-naphthyridin-1-one;
6-(pyridin-2-ylamino)-8-{[2-(pyridin-3-yl)ethyl]amino}-1,2-dihydro-2,7-naphthyridin-1-one;
6-(6-amino-5-methylpyridin-3-yl)-8-({4-[4-(morpholin-4-yl)cyclohexyl]phenyl}amino)-1,2-dihydro-2,7-naphthyridin-1-one;
6-(5-amino-6-methylpyrazin-2-yl)-8-({4-[4-(morpholin-4-yl)cyclohexyl]phenyl}amino)-1,2-dihydro-2,7-naphthyridin-1-one;
6-(1-methyl-1H-pyrazol-4-yl)-8-({4-[4-(morpholin-4-yl)cyclohexyl]phenyl}amino)-1,2-dihydro-2,7-naphthyridin-1-one;
6-(2-amino-4-methylpyrimidin-5-yl)-8-({4-[4-(morpholin-4-yl)cyclohexyl]phenyl}amino)-1,2-dihydro-2,7-naphthyridin-1-one;
6-(6-methoxypyrazin-2-yl)-8-{[10-(2,2,2-trifluoroacetyl)-10-azatricyclo[6.3.1.0̂{2,7}]dodeca-2,4,6-trien-4-yl]amino}-1,2-dihydro-2,7-naphthyridin-1-one;
8-{10-azatricyclo[6.3.1.0̂{2,7}]dodeca-2,4,6-trien-4-ylamino}-6-(6-methoxypyrazin-2-yl)-1,2-dihydro-2,7-naphthyridin-1-one;
6-(pyrimidin-5-yl)-8-{[10-(2,2,2-trifluoroacetyl)-10-azatricyclo[6.3.1.0̂ {2,7}]dodeca-2,4,6-trien-4-yl]amino}-1,2-dihydro-2,7-naphthyridin-1-one;
8-{10-azatricyclo[6.3.1.0̂{2,7}]dodeca-2,4,6-trien-4-ylamino}-6-(pyrimidin-5-yl)-1,2-dihydro-2,7-naphthyridin-1-one;
8-{[4-(1-cyclopropylpiperidin-4-yl)-3-methylphenyl]amino}-6-(3-methoxypropyl)-1,2-dihydro-2,7-naphthyridin-1-one;
6-(3-methoxypropyl)-8-({3-methyl-4-[(2R)-2-methylmorpholin-4-yl]phenyl}amino)-1,2-dihydro-2,7-naphthyridin-1-one;
6-(3-methoxypropyl)-8-{[4-(morpholin-4-yl)phenyl]amino}-1,2-dihydro-2,7-naphthyridin-1-one;
(2E)-3-[1-({6-[(2R)-2-methylmorpholin-4-yl]pyridin-3-yl}amino)-8-oxo-7,8-dihydro-2,7-naphthyridin-3-yl]prop-2-enoic acid;
8-[(2S)-butan-2-ylamino]-6-(pyridin-2-ylamino)-1,2-dihydro-2,7-naphthyridin-1-one;
8-[(cyclopropylmethyl)amino]-6-(pyridin-2-ylamino)-1,2-dihydro-2,7-naphthyridin-1-one;
8-(cyclopentylamino)-6-[(4-methylpyridin-2-yl)amino]-1,2-dihydro-2,7-naphthyridin-1-one;
6-[(4-methylpyridin-2-yl)amino]-8-(oxan-4-ylamino)-1,2-dihydro-2,7-naphthyridin-1-one;
8-{[(2S)-1-hydroxypropan-2-yl]amino}-6-[(4-methylpyridin-2-yl)amino]-1,2-dihydro-2,7-naphthyridin-1-one;
8-{[(2R)-1-hydroxypropan-2-yl]amino}-6-[(4-methylpyridin-2-yl)amino]-1,2-dihydro-2,7-naphthyridin-1-one;
8-(cyclopentylamino)-6-(pyridin-2-ylamino)-1,2-dihydro-2,7-naphthyridin-1-one;
8-(oxan-4-ylamino)-6-(pyridin-2-ylamino)-1,2-dihydro-2,7-naphthyridin-1-one;
8-{[(2S)-1-hydroxypropan-2-yl]amino}-6-(pyridin-2-ylamino)-1,2-dihydro-2,7-naphthyridin-1-one;
8-{[(2R)-1-hydroxypropan-2-yl]amino}-6-(pyridin-2-ylamino)-1,2-dihydro-2,7-naphthyridin-1-one;
8-({4-[4-(4-methylpiperazin-1-yl)cyclohexyl]phenyl}amino)-6-(pyrimidin-5-yl)-1,2-dihydro-2,7-naphthyridin-1-one;
6-(6-methoxypyrazin-2-yl)-8-[(3-methyl-4-{1-[(oxan-4-yl)carbonyl]piperidin-4-yl}phenyl)amino]-1,2-dihydro-2,7-naphthyridin-1-one;
6-[(4-methylpyridin-2-yl)amino]-8-{[2-(1-methylpyrrolidin-2-yl)ethyl]amino}-1,2-dihydro-2,7-naphthyridin-1-one;
8-{[(2S)-1-methoxypropan-2-yl]amino}-6-[(4-methylpyridin-2-yl)amino]-1,2-dihydro-2,7-naphthyridin-1-one;
8-(tert-butylamino)-6-[(4-methylpyridin-2-yl)amino]-1,2-dihydro-2,7-naphthyridin-1-one;
4-fluoro-2-methyl-6-[(4-methylpyridin-2-yl)amino]-8-(propan-2-ylamino)-1,2-dihydro-2,7-naphthyridin-1-one;
ethyl (2E)-3-(1-{[4-(morpholin-4-yl)phenyl]amino}-8-oxo-7,8-dihydro-2,7-naphthyridin-3-yl)prop-2-enoate;
6-(2-ethoxyethyl)-8-{[4-(morpholin-4-yl)phenyl]amino}-1,2-dihydro-2,7-naphthyridin-1-one;
6-(2-hydroxyethyl)-8-{[4-(morpholin-4-yl)phenyl]amino}-1,2-dihydro-2,7-naphthyridin-1-one;
ethyl 3-(1-{[4-(morpholin-4-yl)phenyl]amino}-8-oxo-7,8-dihydro-2,7-naphthyridin-3-yl)propanoate;
8-{[4-(1-cyclopropylpiperidin-4-yl)-3-methylphenyl]amino}-6-(2-hydroxyethyl)-1,2-dihydro-2,7-naphthyridin-1-one;
6-{[4-(hydroxymethyl)pyridin-2-yl]amino}-8-(propan-2-ylamino)-1,2-dihydro-2,7-naphthyridin-1-one;
2-{[8-oxo-1-(propan-2-ylamino)-7,8-dihydro-2,7-naphthyridin-3-yl]amino}pyridine-4-carboxamide;
8-[(2-methylbutan-2-yl)amino]-6-[(4-methylpyridin-2-yl)amino]-1,2-dihydro-2,7-naphthyridin-1-one;
8-[(1-hydroxy-2-methylpropan-2-yl)amino]-6-[(4-methylpyridin-2-yl)amino]-1,2-dihydro-2,7-naphthyridin-1-one;
6-(3-hydroxypropyl)-8-{[4-(morpholin-4-yl)phenyl]amino}-1,2-dihydro-2,7-naphthyridin-1-one;
8-(azetidin-3-ylamino)-6-[(4-methylpyridin-2-yl)amino]-1,2-dihydro-2,7-naphthyridin-1-one;
6-[(4-methylpyridin-2-yl)amino]-8-[(3R)-oxolan-3-ylamino]-1,2-dihydro-2,7-naphthyridin-1-one;
6-[(4-methylpyridin-2-yl)amino]-8-[(3S)-oxolan-3-ylamino]-1,2-dihydro-2,7-naphthyridin-1-one;
8-[(4-hydroxycyclohexyl)amino]-6-[(4-methylpyridin-2-yl)amino]-1,2-dihydro-2,7-naphthyridin-1-one;
6-(2-aminopyrimidin-5-yl)-8-{[(2S)-1-methoxypropan-2-yl]amino}-1,2-dihydro-2,7-naphthyridin-1-one;
6-(2-aminopyrimidin-5-yl)-8-{[(2S)-2-(3-chlorophenyl)-2-hydroxyethyl]amino}-1,2-dihydro-2,7-naphthyridin-1-one;
6-(2-aminopyrimidin-5-yl)-8-[(2,2,2-trifluoroethyl)amino]-1,2-dihydro-2,7-naphthyridin-1-one;
6-(2-aminopyrimidin-5-yl)-8-{[(2S)-2-hydroxypropyl]amino}-1,2-dihydro-2,7-naphthyridin-1-one;
6-(2-aminopyrimidin-5-yl)-8-{[(2R)-2-hydroxypropyl]amino}-1,2-dihydro-2,7-naphthyridin-1-one;
8-{[(2S)-2-(3-chlorophenyl)-2-hydroxyethyl]amino}-6-[(4-methylpyridin-2-yl)amino]-1,2-dihydro-2,7-naphthyridin-1-one;
8-[(3R)-3-hydroxypyrrolidin-1-yl]-6-[(4-methylpyridin-2-yl)amino]-1,2-dihydro-2,7-naphthyridin-1-one;
8-[(3S)-3-(hydroxymethyl)pyrrolidin-1-yl]-6-[(4-methylpyridin-2-yl)amino]-1,2-dihydro-2,7-naphthyridin-1-one;
8-[(3R)-3-(hydroxymethyl)pyrrolidin-1-yl]-6-[(4-methylpyridin-2-yl)amino]-1,2-dihydro-2,7-naphthyridin-1-one;
6-(2-aminopyrimidin-5-yl)-8-(cyclobutylamino)-1,2-dihydro-2,7-naphthyridin-1-one;
6-(2-aminopyrimidin-5-yl)-8-[(2-methylbutan-2-yl)amino]-1,2-dihydro-2,7-naphthyridin-1-one;
6-(2-aminopyrimidin-5-yl)-8-(tert-butylamino)-1,2-dihydro-2,7-naphthyridin-1-one;
2-ethyl-6-[(4-methylpyridin-2-yl)amino]-8-(propan-2-ylamino)-1,2-dihydro-2,7-naphthyridin-1-one;
2-ethyl-6-[(4-ethylpyridin-2-yl)amino]-8-(propan-2-ylamino)-1,2-dihydro-2,7-naphthyridin-1-one;
6-[(4-chloropyridin-2-yl)amino]-2-ethyl-8-(propan-2-ylamino)-1,2-dihydro-2,7-naphthyridin-1-one;
2-ethyl-8-(propan-2-ylamino)-6-[(4-propylpyridin-2-yl)amino]-1,2-dihydro-2,7-naphthyridin-1-one;
2-ethyl-8-(propan-2-ylamino)-6-(pyrazin-2-ylamino)-1,2-dihydro-2,7-naphthyridin-1-one;
2-methyl-6-[(4-methylpyridin-2-yl)amino]-8-{[4-(piperidin-4-yl)phenyl]amino}-1,2-dihydro-2,7-naphthyridin-1-one;
2-methyl-8-{[4-(1-methylpiperidin-4-yl)phenyl]amino}-6-[(4-methylpyridin-2-yl)amino]-1,2-dihydro-2,7-naphthyridin-1-one;
8-({4-[1-acetylpiperidin-4-yl)phenyl]amino}-2-methyl-6-[(4-methylpyridin-2-yl)amino]-1,2-dihydro-2,7-naphthyridin-1-one;
8-({4-[1-(2-methanesulfonylethyl)piperidin-4-yl]phenyl}amino)-2-methyl-6-[(4-methylpyridin-2-yl)amino]-1,2-dihydro-2,7-naphthyridin-1-one;
3-{4-[4-({7-methyl-3-[(4-methylpyridin-2-yl)amino]-8-oxo-7,8-dihydro-2,7-naphthyridin-1-yl}amino)phenyl]piperidin-1-yl}propanenitrile;
2-(2-hydroxyethyl)-6-[(4-methylpyridin-2-yl)amino]-8-(propan-2-ylamino)-1,2-dihydro-2,7-naphthyridin-1-one;
2-(2-methoxyethyl)-6-[(4-methylpyridin-2-yl)amino]-8-(propan-2-ylamino)-1,2-dihydro-2,7-naphthyridin-1-one;
6-[(4-methylpyridin-2-yl)amino]-2-[2-(morpholin-4-yl)ethyl]-8-(propan-2-ylamino)-1,2-dihydro-2,7-naphthyridin-1-one;
N-ethyl-2-{6-[(4-methylpyridin-2-yl)amino]-1-oxo-8-(propan-2-ylamino)-1,2-dihydro-2,7-naphthyridin-2-yl}acetamide;
2-(2-hydroxyethyl)-8-(propan-2-ylamino)-6-(pyrazin-2-ylamino)-1,2-dihydro-2,7-naphthyridin-1-one;
2-(2-methoxyethyl)-8-(propan-2-ylamino)-6-(pyrazin-2-ylamino)-1,2-dihydro-2,7-naphthyridin-1-one;
2-[2-(morpholin-4-yl)ethyl]-8-(propan-2-ylamino)-6-(pyrazin-2-ylamino)-1,2-dihydro-2,7-naphthyridin-1-one;
N-ethyl-2-[1-oxo-8-(propan-2-ylamino)-6-(pyrazin-2-ylamino)-1,2-dihydro-2,7-naphthyridin-2-yl]acetamide;
8-{[(2S)-1-hydroxypropan-2-yl]amino}-2-methyl-6-[(4-methylpyridin-2-yl)amino]-1,2-dihydro-2,7-naphthyridin-1-one;
8-{[(1S,2R)-2-hydroxycyclopentyl]amino}-2-methyl-6-[(4-methylpyridin-2-yl)amino]-1,2-dihydro-2,7-naphthyridin-1-one;
2-methyl-6-[(4-methylpyridin-2-yl)amino]-8-[(3S)-oxolan-3-ylamino]-1,2-dihydro-2,7-naphthyridin-1-one;
8-[(4-hydroxycyclohexyl)amino]-2-methyl-6-[(4-methylpyridin-2-yl)amino]-1,2-dihydro-2,7-naphthyridin-1-one;
8-{[(2S)-1-hydroxypropan-2-yl]amino}-2-methyl-6-(pyrazin-2-ylamino)-1,2-dihydro-2,7-naphthyridin-1-one;
8-{[(1S,2R)-2-hydroxycyclopentyl]amino}-2-methyl-6-(pyrazin-2-ylamino)-1,2-dihydro-2,7-naphthyridin-1-one;
2-methyl-8-[(3S)-oxolan-3-ylamino]-6-(pyrazin-2-ylamino)-1,2-dihydro-2,7-naphthyridin-1-one;
8-[(4-hydroxycyclohexyl)amino]-2-methyl-6-(pyrazin-2-ylamino)-1,2-dihydro-2,7-naphthyridin-1-one;
8-[(3-hydroxypropyl)amino]-2-methyl-6-[(4-methylpyridin-2-yl)amino]-1,2-dihydro-2,7-naphthyridin-1-one;
2-methyl-6-[(4-methylpyridin-2-yl)amino]-8-[(2,2,2-trifluoroethyl)amino]-1,2-dihydro-2,7-naphthyridin-1-one;
8-(tert-butylamino)-2-methyl-6-[(4-methylpyridin-2-yl)amino]-1,2-dihydro-2,7-naphthyridin-1-one;
8-[(1-hydroxy-2-methylpropan-2-yl)amino]-2-methyl-6-[(4-methylpyridin-2-yl)amino]-1,2-dihydro-2,7-naphthyridin-1-one;
8-(cyclobutylamino)-2-methyl-6-[(4-methylpyridin-2-yl)amino]-1,2-dihydro-2,7-naphthyridin-1-one;
2-methyl-6-[(4-methylpyridin-2-yl)amino]-8-{[(2R)-oxolan-2-ylmethyl]amino}-1,2-dihydro-2,7-naphthyridin-1-one;
8-{[(2R)-2-hydroxypropyl]amino}-2-methyl-6-[(4-methylpyridin-2-yl)amino]-1,2-dihydro-2,7-naphthyridin-1-one;
2-methyl-6-[(4-methylpyridin-2-yl)amino]-8-({4-[(morpholin-4-yl)carbonyl]phenyl}amino)-1,2-dihydro-2,7-naphthyridin-1-one;
N-[2-(diethylamino)ethyl]-4-({7-methyl-3-[(4-methylpyridin-2-yl)amino]-8-oxo-7,8-dihydro-2,7-naphthyridin-1-yl}amino)benzamide;
8-[(4-methanesulfonylphenyl)amino]-2-methyl-6-[(4-methylpyridin-2-yl)amino]-1,2-dihydro-2,7-naphthyridin-1-one;
3-({7-methyl-3-[(4-methylpyridin-2-yl)amino]-8-oxo-7,8-dihydro-2,7-naphthyridin-1-yl}amino)benzene-1-sulfonamide;
8-{[4-(2-ethoxyethoxy)phenyl]amino}-2-methyl-6-[(4-methylpyridin-2-yl)amino]-1,2-dihydro-2,7-naphthyridin-1-one;
8-{[3-(2-ethoxyethoxy)phenyl]amino}-2-methyl-6-[(4-methylpyridin-2-yl)amino]-1,2-dihydro-2,7-naphthyridin-1-one;
8-[(4-methanesulfonylphenyl)amino]-2-methyl-6-(pyrazin-2-ylamino)-1,2-dihydro-2,7-naphthyridin-1-one;
8-{[3-(2-ethoxyethoxy)phenyl]amino}-2-methyl-6-(pyrazin-2-ylamino)-1,2-dihydro-2,7-naphthyridin-1-one;
8-({4-[4-(dimethylamino)cyclohexyl]phenyl}amino)-6-(2-methylpyrimidin-5-yl)-1,2-dihydro-2,7-naphthyridin-1-one;
3-[4-(2-methyl-4-{[3-(2-methylpyrimidin-5-yl)-8-oxo-7,8-dihydro-2,7-naphthyridin-1-yl]amino}-phenyl)piperidin-1-yl]-1$1̂{6}-thietane-1,1-dione;
8-({4-[4-(4-ethylpiperazin-1-yl)cyclohexyl]phenyl}amino)-6-(pyrimidin-5-yl)-1,2-dihydro-2,7-naphthyridin-1-one;
8-({4-[4-(4-methylpiperazin-1-yl)cyclohexyl]phenyl}amino)-6-(pyrimidin-5-yl)-1,2-dihydro-2,7-naphthyridin-1-one;
8-({4-[4-(dimethylamino)cyclohexyl]phenyl}amino)-6-(6-methoxypyrazin-2-yl)-1,2-dihydro-2,7-naphthyridin-1-one;
N-[2-(diethylamino)ethyl]-4-{[7-methyl-8-oxo-3-(pyrazin-2-ylamino)-7,8-dihydro-2,7-naphthyridin-1-yl]amino}benzamide;
3-{[7-methyl-8-oxo-3-(pyrazin-2-ylamino)-7,8-dihydro-2,7-naphthyridin-1-yl]amino}benzene-1-sulfonamide;
8-{[4-(2-ethoxyethoxy)phenyl]amino}-2-methyl-6-(pyrazin-2-ylamino)-1,2-dihydro-2,7-naphthyridin-1-one;
2-methyl-8-{[4-(piperidin-4-yl)phenyl]amino}-6-(pyrazin-2-ylamino)-1,2-dihydro-2,7-naphthyridin-1-one;
N-methyl-4-({7-methyl-3-[(4-methylpyridin-2-yl)amino]-8-oxo-7,8-dihydro-2,7-naphthyridin-1-yl}amino)benzamide;
N-methyl-4-{[7-methyl-8-oxo-3-(pyrazin-2-ylamino)-7,8-dihydro-2,7-naphthyridin-1-yl]amino}benzamide;
2-methyl-8-{[4-(1-methylpiperidin-4-yl)phenyl]amino}-6-(pyrazin-2-ylamino)-1,2-dihydro-2,7-naphthyridin-1-one;
8-{[4-(1-acetylpiperidin-4-yl)phenyl]amino}-2-methyl-6-(pyrazin-2-ylamino)-1,2-dihydro-2,7-naphthyridin-1-one;
2-methyl-8-({4-[1-(oxetan-3-yl)piperidin-4-yl]phenyl}amino)-6-(pyrazin-2-ylamino)-1,2-dihydro-2,7-naphthyridin-1-one;
2-methyl-8-(propan-2-ylamino)-6-(pyrazin-2-ylamino)-1,2-dihydro-2,7-naphthyridin-1-one;
2-ethyl-6-[(3-methylpyrazin-2-yl)amino]-8-(propan-2-ylamino)-1,2-dihydro-2,7-naphthyridin-1-one;
8-[(3-hydroxypropyl)amino]-2-methyl-6-(pyrazin-2-ylamino)-1,2-dihydro-2,7-naphthyridin-1-one;
2-methyl-6-(pyrazin-2-ylamino)-8-[(2,2,2-trifluoroethyl)amino]-1,2-dihydro-2,7-naphthyridin-1-one;
8-(tert-butylamino)-2-methyl-6-(pyrazin-2-ylamino)-1,2-dihydro-2,7-naphthyridin-1-one;
8-[(1-hydroxy-2-methylpropan-2-yl)amino]-2-methyl-6-(pyrazin-2-ylamino)-1,2-dihydro-2,7-naphthyridin-1-one;
8-(cyclobutylamino)-2-methyl-6-(pyrazin-2-ylamino)-1,2-dihydro-2,7-naphthyridin-1-one;
8-(tert-butylamino)-2-(2-hydroxyethyl)-6-[(4-methylpyridin-2-yl)amino]-1,2-dihydro-2,7-naphthyridin-1-one;
8-(tert-butylamino)-2-(2-hydroxyethyl)-6-(pyrazin-2-ylamino)-1,2-dihydro-2,7-naphthyridin-1-one;
8-[(1-hydroxy-2-methylpropan-2-yl)amino]-2-(2-hydroxyethyl)-6-[(4-methylpyridin-2-yl)amino]-1,2-dihydro-2,7-naphthyridin-1-one;
8-[(1-hydroxy-2-methylpropan-2-yl)amino]-2-(2-hydroxyethyl)-6-(pyrazin-2-ylamino)-1,2-dihydro-2,7-naphthyridin-1-one;
N-methyl-4-({7-methyl-3-[(4-methylpyridin-2-yl)amino]-8-oxo-7,8-dihydro-2,7-naphthyridin-1-yl}amino)benzene-1-sulfonamide;
N-methyl-4-{[7-methyl-8-oxo-3-(pyrazin-2-ylamino)-7,8-dihydro-2,7-naphthyridin-1-yl]amino}benzene-1-sulfonamide;
8-[(4-methanesulfonylphenyl)amino]-2-methyl-6-[(4-methylpyridin-2-yl)amino]-1,2-dihydro-2,7-naphthyridin-1-one;
2-ethyl-8-{[(2S)-1-hydroxypropan-2-yl]amino}-6-(pyrazin-2-ylamino)-1,2-dihydro-2,7-naphthyridin-1-one;
2-ethyl-8-{[(1S,2R)-2-hydroxycyclopentyl]amino}-6-(pyrazin-2-ylamino)-1,2-dihydro-2,7-naphthyridin-1-one;
2-ethyl-8-[(3S)-oxolan-3-ylamino]-6-(pyrazin-2-ylamino)-1,2-dihydro-2,7-naphthyridin-1-one;
2-ethyl-8-[(4-hydroxycyclohexyl)amino]-6-(pyrazin-2-ylamino)-1,2-dihydro-2,7-naphthyridin-1-one;
8-[(4-hydroxycyclohexyl)amino]-2-methyl-6-[(4-methylpyridin-2-yl)amino]-1,2-dihydro-2,7-naphthyridin-1-one;
8-[(4-hydroxycyclohexyl)amino]-2-methyl-6-(pyrazin-2-ylamino)-1,2-dihydro-2,7-naphthyridin-1-one;
2-ethyl-8-{[(2S)-1-hydroxypropan-2-yl]amino}-6-[(4-methylpyridin-2-yl)amino]-1,2-dihydro-2,7-naphthyridin-1-one;
2-ethyl-8-{[(1S,2R)-2-hydroxycyclopentyl]amino}-6-[(4-methylpyridin-2-yl)amino]-1,2-dihydro-2,7-naphthyridin-1-one;
2-ethyl-6-[(4-methylpyridin-2-yl)amino]-8-[(3S)-oxolan-3-ylamino]-1,2-dihydro-2,7-naphthyridin-1-one;
2-ethyl-8-[(4-hydroxycyclohexyl)amino]-6-[(4-methylpyridin-2-yl)amino]-1,2-dihydro-2,7-naphthyridin-1-one;
4-({7-methyl-3-[(4-methylpyridin-2-yl)amino]-8-oxo-7,8-dihydro-2,7-naphthyridin-1-yl}amino)-N-propylbenzamide;
8-[(4-methoxyphenyl)amino]-2-methyl-6-[(4-methylpyridin-2-yl)amino]-1,2-dihydro-2,7-naphthyridin-1-one;
8-[(4-methoxyphenyl)amino]-2-methyl-6-(pyrazin-2-ylamino)-1,2-dihydro-2,7-naphthyridin-1-one;
8-(tert-butylamino)-2-ethyl-6-[(4-methylpyridin-2-yl)amino]-1,2-dihydro-2,7-naphthyridin-1-one;
8-(tert-butylamino)-2-ethyl-6-(pyrazin-2-ylamino)-1,2-dihydro-2,7-naphthyridin-1-one;
2-ethyl-8-[(1-hydroxy-2-methylpropan-2-yl)amino]-6-[(4-methylpyridin-2-yl)amino]-1,2-dihydro-2,7-naphthyridin-1-one;
2-ethyl-8-[(1-hydroxy-2-methylpropan-2-yl)amino]-6-(pyrazin-2-ylamino)-1,2-dihydro-2,7-naphthyridin-1-one;
3-({7-methyl-3-[(4-methylpyridin-2-yl)amino]-8-oxo-7,8-dihydro-2,7-naphthyridin-1-yl}amino)benzamide;
8-[(1-hydroxy-2-methylpropan-2-yl)amino]-2-methyl-6-[(4-methylpyridin-2-yl)amino]-1,2-dihydro-2,7-naphthyridin-1-one;
8-(tert-butylamino)-2-methyl-6-(pyrazin-2-ylamino)-1,2-dihydro-2,7-naphthyridin-1-one;
4-{[7-methyl-8-oxo-3-(pyrazin-2-ylamino)-7,8-dihydro-2,7-naphthyridin-1-yl]amino}-N-propylbenzamide;
2-(2-hydroxyethyl)-8-{[(2S)-1-hydroxypropan-2-yl]amino}-6-[(4-methylpyridin-2-yl)amino]-1,2-dihydro-2,7-naphthyridin-1-one;
2-(2-hydroxyethyl)-6-[(4-methylpyridin-2-yl)amino]-8-[(3S)-oxolan-3-ylamino]-1,2-dihydro-2,7-naphthyridin-1-one;
8-{[(1S,2R)-2-hydroxycyclopentyl]amino}-2-(2-hydroxyethyl)-6-[(4-methylpyridin-2-yl)amino]-1,2-dihydro-2,7-naphthyridin-1-one;
8-[(4-hydroxycyclohexyl)amino]-2-(2-hydroxyethyl)-6-[(4-methylpyridin-2-yl)amino]-1,2-dihydro-2,7-naphthyridin-1-one;
8-[(3,4-dimethoxyphenyl)amino]-6-[(4-methylpyridin-2-yl)amino]-1,2-dihydro-2,7-naphthyridin-1-one;
2-methyl-6-[(4-methylpyridin-2-yl)amino]-8-{[6-(oxan-4-yl)pyridin-3-yl]amino}-1,2-dihydro-2,7-naphthyridin-1-one;
2-(2-hydroxyethyl)-{8-[(2S)-1-hydroxypropan-2-yl]amino}-6-(pyrazin-2-ylamino)-1,2-dihydro-2,7-naphthyridin-1-one;
8-{[(1S,2R)-2-hydroxycyclopentyl]amino}-2-(2-hydroxyethyl)-6-(pyrazin-2-ylamino)-1,2-dihydro-2,7-naphthyridin-1-one;
2-(2-hydroxyethyl)-8-[(3S)-oxolan-3-ylamino]-6-(pyrazin-2-ylamino)-1,2-dihydro-2,7-naphthyridin-1-one;
8-[(4-hydroxycyclohexyl)amino]-2-(2-hydroxyethyl)-6-(pyrazin-2-ylamino)-1,2-dihydro-2,7-naphthyridin-1-one;
8-[(4-hydroxycyclohexyl)amino]-2-methyl-6-(pyrazin-2-ylamino)-1,2-dihydro-2,7-naphthyridin-1-one;
8-[(4-hydroxycyclohexyl)amino]-2-methyl-6-[(4-methylpyridin-2-yl)amino]-1,2-dihydro-2,7-naphthyridin-1-one;
8-{[(2R)-2-hydroxypropyl]amino}-2-methyl-6-(pyrazin-2-ylamino)-1,2-dihydro-2,7-naphthyridin-1-one;
2-methyl-8-{[(2R)-oxolan-2-ylmethyl]amino}-6-(pyrazin-2-ylamino)-1,2-dihydro-2,7-naphthyridin-1-one;
3-{[7-methyl-8-oxo-3-(pyrazin-2-ylamino)-7,8-dihydro-2,7-naphthyridin-1-yl]amino}benzamide;
4-{[7-methyl-8-oxo-3-(pyrazin-2-ylamino)-7,8-dihydro-2,7-naphthyridin-1-yl]amino}benzamide;
2-methyl-8-({4-[(morpholin-4-yl)carbonyl]phenyl}amino)-6-(pyrazin-2-ylamino)-1,2-dihydro-2,7-naphthyridin-1-one;
6-[(4-chloropyridin-2-yl)amino]-8-[(4-hydroxycyclohexyl)amino]-2-methyl-1,2-dihydro-2,7-naphthyridin-1-one;
N-ethyl-4-({7-methyl-3-[(4-methylpyridin-2-yl)amino]-8-oxo-7,8-dihydro-2,7-naphthyridin-1-yl}amino)benzamide;
N-ethyl-4-{[7-methyl-8-oxo-3-(pyrazin-2-ylamino)-7,8-dihydro-2,7-naphthyridin-1-yl]amino}benzamide;
4-({3-[(4-chloropyridin-2-yl)amino]-7-methyl-8-oxo-7,8-dihydro-2,7-naphthyridin-1-yl}amino)-N-ethylbenzamide;
6-[(4-chloropyridin-2-yl)amino]-2-ethyl-8-[(4-hydroxycyclohexyl)amino]-1,2-dihydro-2,7-naphthyridin-1-one;
6-[(4-chloropyridin-2-yl)amino]-8-[(4-hydroxycyclohexyl)amino]-2-(2-hydroxyethyl)-1,2-dihydro-2,7-naphthyridin-1-one;
6-[(4-chloropyridin-2-yl)amino]-8-[(4-hydroxycyclohexyl)amino]-2-methyl-1,2-dihydro-2,7-naphthyridin-1-one;
2-[2-(dimethylamino)ethyl]-8-[(4-hydroxycyclohexyl)amino]-6-[(4-methylpyridin-2-yl)amino]-1,2-dihydro-2,7-naphthyridin-1-one;
2-[2-(dimethylamino)ethyl]-8-[(4-hydroxycyclohexyl)amino]-6-(pyrazin-2-ylamino)-1,2-dihydro-2,7-naphthyridin-1-one;
6-[(4-chloropyridin-2-yl)amino]-2-[2-(dimethylamino)ethyl]-8-[(4-hydroxycyclohexyl)amino]-1,2-dihydro-2,7-naphthyridin-1-one;
8-[(3-methanesulfonylphenyl)amino]-2-methyl-6-[(4-methylpyridin-2-yl)amino]-1,2-dihydro-2,7-naphthyridin-1-one;
8-[(3-methanesulfonylphenyl)amino]-2-methyl-6-(pyrazin-2-ylamino)-1,2-dihydro-2,7-naphthyridin-1-one;
8-[(4-hydroxycyclohexyl)amino]-2-(2-hydroxyethyl)-6-[(4-methylpyridin-2-yl)amino]-1,2-dihydro-2,7-naphthyridin-1-one;
2-ethyl-8-[(4-hydroxycyclohexyl)amino]-6-[(5-methylpyrazin-2-yl)amino]-1,2-dihydro-2,7-naphthyridin-1-one;
2-ethyl-6-[(5-fluoro-4-methylpyridin-2-yl)amino]-8-[(4-hydroxycyclohexyl)amino]-1,2-dihydro-2,7-naphthyridin-1-one;
2-ethyl-8-{[(2S)-1-hydroxypropan-2-yl]amino}-6-[(5-methylpyrazin-2-yl)amino]-1,2-dihydro-2,7-naphthyridin-1-one;
2-ethyl-6-[(5-fluoro-4-methylpyridin-2-yl)amino]-8-{[(2S)-1-hydroxypropan-2-yl]amino}-1,2-dihydro-2,7-naphthyridin-1-one;
2-[2-(dimethylamino)ethyl]-8-[(4-methanesulfonylphenyl)amino]-6-[(4-methylpyridin-2-yl)amino]-1,2-dihydro-2,7-naphthyridin-1-one;
2-[2-(dimethylamino)ethyl]-8-[(4-methanesulfonylphenyl)amino]-6-(pyrazin-2-ylamino)-1,2-dihydro-2,7-naphthyridin-1-one;
8-{[(2S)-1-hydroxypropan-2-yl]amino}-2-methyl-6-[(4-methylpyridin-2-yl)amino]-1,2-dihydro-2,7-naphthyridin-1-one;
8-(cyclopropylamino)-2-methyl-6-(pyrazin-2-ylamino)-1,2-dihydro-2,7-naphthyridin-1-one;
6-[(4-chloropyridin-2-yl)amino]-8-(cyclopropylamino)-2-methyl-1,2-dihydro-2,7-naphthyridin-1-one;
6-[(5-chloro-4-methylpyridin-2-yl)amino]-8-(cyclopropylamino)-2-methyl-1,2-dihydro-2,7-naphthyridin-1-one;
8-(cyclopropylamino)-6-[(5-fluoro-4-methylpyridin-2-yl)amino]-2-methyl-1,2-dihydro-2,7-naphthyridin-1-one;
6-[(4-chloropyridin-2-yl)amino]-8-(cyclobutylamino)-2-methyl-1,2-dihydro-2,7-naphthyridin-1-one;
8-(tert-butylamino)-2-methyl-6-(pyrazin-2-ylamino)-1,2-dihydro-2,7-naphthyridin-1-one;
8-(tert-butylamino)-6-[(4-chloropyridin-2-yl)amino]-2-methyl-1,2-dihydro-2,7-naphthyridin-1-one;
8-(tert-butylamino)-6-[(5-chloro-4-methylpyridin-2-yl)amino]-2-methyl-1,2-dihydro-2,7-naphthyridin-1-one;
8-(tert-butylamino)-6-[(5-fluoro-4-methylpyridin-2-yl)amino]-2-methyl-1,2-dihydro-2,7-naphthyridin-1-one;
6-[(5-chloro-4-methylpyridin-2-yl)amino]-8-[(1-hydroxy-2-methylpropan-2-yl)amino]-2-(2-hydroxyethyl)-1,2-dihydro-2,7-naphthyridin-1-one;
6-[(5-fluoro-4-methylpyridin-2-yl)amino]-8-[(1-hydroxy-2-methylpropan-2-yl)amino]-2-(2-hydroxyethyl)-1,2-dihydro-2,7-naphthyridin-1-one;
8-[(1-hydroxy-2-methylpropan-2-yl)amino]-2-methyl-6-[(4-methylpyridin-2-yl)amino]-1,2-dihydro-2,7-naphthyridin-1-one;
6-[(5-fluoro-4-methylpyridin-2-yl)amino]-8-[(1-hydroxy-2-methylpropan-2-yl)amino]-2-methyl-1,2-dihydro-2,7-naphthyridin-1-one;
2-methyl-8-{[(2S)-oxolan-2-ylmethyl]amino}-6-(pyrazin-2-ylamino)-1,2-dihydro-2,7-naphthyridin-1-one;
2-ethyl-6-(pyrazin-2-ylamino)-8-[(2,2,2-trifluoroethyl)amino]-1,2-dihydro-2,7-naphthyridin-1-one;
8-(propan-2-ylamino)-6-{[4-(pyridin-3-yl)pyridin-2-yl]amino}-1,2-dihydro-2,7-naphthyridin-1-one;
2-methyl-8-(propan-2-ylamino)-6-{[4-(pyridin-3-yl)pyridin-2-yl]amino}-1,2-dihydro-2,7-naphthyridin-1-one;
2-ethyl-8-{[(2S)-1-hydroxybutan-2-yl]amino}-6-(pyrazin-2-ylamino)-1,2-dihydro-2,7-naphthyridin-1-one;
2-ethyl-8-{[(2S)-1-hydroxy-3-methylbutan-2-yl]amino}-6-[(4-methylpyridin-2-yl)amino]-1,2-dihydro-2,7-naphthyridin-1-one;
2-ethyl-8-{[(2S)-1-hydroxy-3-methylbutan-2-yl]amino}-6-(pyrazin-2-ylamino)-1,2-dihydro-2,7-naphthyridin-1-one;
8-{[(2S)-1-hydroxypropan-2-yl]amino}-2-methyl-6-(pyrazin-2-ylamino)-1,2-dihydro-2,7-naphthyridin-1-one;
6-[(4-chloropyridin-2-yl)amino]-8-{[(2S)-1-hydroxypropan-2-yl]amino}-2-methyl-1,2-dihydro-2,7-naphthyridin-1-one;
2-(2-hydroxyethyl)-8-[(4-methanesulfonylphenyl)amino]-6-[(4-methylpyridin-2-yl)amino]-1,2-dihydro-2,7-naphthyridin-1-one;
2-(2-hydroxyethyl)-8-[(4-methanesulfonylphenyl)amino]-6-(pyrazin-2-ylamino)-1,2-dihydro-2,7-naphthyridin-1-one;
2-ethyl-8-{[(1S,2S)-2-hydroxycyclohexyl]amino}-6-[(4-methylpyridin-2-yl)amino]-1,2-dihydro-2,7-naphthyridin-1-one;
2-ethyl-8-{[(1S,2S)-2-hydroxycyclohexyl]amino}-6-(pyrazin-2-ylamino)-1,2-dihydro-2,7-naphthyridin-1-one;
2-ethyl-8-{[(1S,2R)-2-hydroxycyclohexyl]amino}-6-[(4-methylpyridin-2-yl)amino]-1,2-dihydro-2,7-naphthyridin-1-one;
2-ethyl-8-{[(1S,2R)-2-hydroxycyclohexyl]amino}-6-(pyrazin-2-ylamino)-1,2-dihydro-2,7-naphthyridin-1-one;
6-[(4-chloropyridin-2-yl)amino]-2-methyl-8-{[(2S)-oxolan-2-ylmethyl]amino}-1,2-dihydro-2,7-naphthyridin-1-one;
2-ethyl-8-({[(1S,3S)-3-hydroxycyclopentyl]methyl}amino)-6-[(4-methylpyridin-2-yl)amino]-1,2-dihydro-2,7-naphthyridin-1-one;
2-ethyl-8-({[(1S,3S)-3-hydroxycyclopentyl]methyl}amino)-6-(pyrazin-2-ylamino)-1,2-dihydro-2,7-naphthyridin-1-one;
2-ethyl-8-({[(1R,3R)-3-hydroxycyclopentyl]methyl}amino)-6-[(4-methylpyridin-2-yl)amino]-1,2-dihydro-2,7-naphthyridin-1-one;
2-ethyl-8-({[(1R,3R)-3-hydroxycyclopentyl]methyl}amino)-6-(pyrazin-2-ylamino)-1,2-dihydro-2,7-naphthyridin-1-one;
6-[(4-chloropyridin-2-yl)amino]-2-ethyl-8-[(2,2,2-trifluoroethyl)amino]-1,2-dihydro-2,7-naphthyridin-1-one;
2-(2-hydroxyethyl)-6-(pyrazin-2-ylamino)-8-[(2,2,2-trifluoroethyl)amino]-1,2-dihydro-2,7-naphthyridin-1-one;
6-[(4-chloropyridin-2-yl)amino]-2-(2-hydroxyethyl)-8-[(2,2,2-trifluoroethyl)amino]-1,2-dihydro-2,7-naphthyridin-1-one;
6-[(4-phenylpyridin-2-yl)amino]-8-(propan-2-ylamino)-1,2-dihydro-2,7-naphthyridin-1-one;
6-{[4-(1-methyl-1H-pyrazol-4-yl)pyridin-2-yl]amino}-8-(propan-2-ylamino)-1,2-dihydro-2,7-naphthyridin-1-one;
2-methyl-6-{[4-(1-methyl-1H-pyrazol-4-yl)pyridin-2-yl]amino}-8-(propan-2-ylamino)-1,2-dihydro-2,7-naphthyridin-1-one;
2-ethyl-8-{[(2S)-1-hydroxybutan-2-yl]amino}-6-[(4-methylpyridin-2-yl)amino]-1,2-dihydro-2,7-naphthyridin-1-one;
2-ethyl-8-{[(2R)-1-hydroxybutan-2-yl]amino}-6-[(4-methylpyridin-2-yl)amino]-1,2-dihydro-2,7-naphthyridin-1-one;
2-ethyl-8-{[(2R)-1-hydroxybutan-2-yl]amino}-6-(pyrazin-2-ylamino)-1,2-dihydro-2,7-naphthyridin-1-one;
2-(2-hydroxyethyl)-8-[(1-methylcyclobutyl)amino]-6-[(4-methylpyridin-2-yl)amino]-1,2-dihydro-2,7-naphthyridin-1-one;
2-(2-hydroxyethyl)-8-[(1-methylcyclobutyl)amino]-6-(pyrazin-2-ylamino)-1,2-dihydro-2,7-naphthyridin-1-one;
2-ethyl-8-({[(1R,3S)-3-hydroxy-3-methylcyclopentyl]methyl}amino)-6-[(4-methylpyridin-2-yl)amino]-1,2-dihydro-2,7-naphthyridin-1-one;
2-ethyl-8-({[(1R,3S)-3-hydroxy-3-methylcyclopentyl]methyl}amino)-6-(pyrazin-2-ylamino)-1,2-dihydro-2,7-naphthyridin-1-one;
2-ethyl-8-({[(1S,3R)-3-hydroxy-3-methylcyclopentyl]methyl}amino)-6-[(4-methylpyridin-2-yl)amino]-1,2-dihydro-2,7-naphthyridin-1-one;
2-ethyl-8-({[(1S,3R)-3-hydroxy-3-methylcyclopentyl]methyl}amino)-6-(pyrazin-2-ylamino)-1,2-dihydro-2,7-naphthyridin-1-one;
2-ethyl-8-{[(1R,2S)-2-hydroxycyclopentyl]amino}-6-[(4-methylpyridin-2-yl)amino]-1,2-dihydro-2,7-naphthyridin-1-one;
2-ethyl-8-{[(1R,2S)-2-hydroxycyclopentyl]amino}-6-(pyrazin-2-ylamino)-1,2-dihydro-2,7-naphthyridin-1-one;
2-ethyl-8-{[(1S,2R)-2-hydroxycyclopentyl]amino}-6-[(4-methylpyridin-2-yl)amino]-1,2-dihydro-2,7-naphthyridin-1-one;
2-ethyl-8-{[(1S,2R)-2-hydroxycyclopentyl]amino}-6-(pyrazin-2-ylamino)-1,2-dihydro-2,7-naphthyridin-1-one;
2-ethyl-8-{[(1S,2S)-2-hydroxycyclopentyl]amino}-6-[(4-methylpyridin-2-yl)amino]-1,2-dihydro-2,7-naphthyridin-1-one;
2-ethyl-8-{[(1S,2S)-2-hydroxycyclopentyl]amino}-6-(pyrazin-2-ylamino)-1,2-dihydro-2,7-naphthyridin-1-one;
2-ethyl-8-{[(1S,3R)-3-hydroxycyclopentyl]amino}-6-[(4-methylpyridin-2-yl)amino]-1,2-dihydro-2,7-naphthyridin-1-one;
2-ethyl-8-{[(1S,3R)-3-hydroxycyclopentyl]amino}-6-(pyrazin-2-ylamino)-1,2-dihydro-2,7-naphthyridin-1-one;
2-ethyl-8-{[(1R,3S)-3-hydroxycyclopentyl]amino}-6-[(4-methylpyridin-2-yl)amino]-1,2-dihydro-2,7-naphthyridin-1-one;
2-ethyl-8-{[(1R,3S)-3-hydroxycyclopentyl]amino}-6-(pyrazin-2-ylamino)-1,2-dihydro-2,7-naphthyridin-1-one;
8-(tert-butylamino)-6-({4-[(3-hydroxyazetidin-1-yl)methyl]pyridin-2-yl}amino)-2-(2-hydroxyethyl)-1,2-dihydro-2,7-naphthyridin-1-one;
8-(tert-butylamino)-6-({4-[(3-hydroxy-3-methylazetidin-1-yl)methyl]pyridin-2-yl}amino)-2-(2-hydroxyethyl)-1,2-dihydro-2,7-naphthyridin-1-one;
8-(tert-butylamino)-2-(2-hydroxyethyl)-6-{[4-(2,2,2-trifluoro-1-hydroxyethyl)pyridin-2-yl]amino}-1,2-dihydro-2,7-naphthyridin-1-one;
8-(tert-butylamino)-2-(2-hydroxyethyl)-6-{[4-(morpholin-4-ylmethyl)pyridin-2-yl]amino}-1,2-dihydro-2,7-naphthyridin-1-one;
8-(tert-butylamino)-2-(2-hydroxyethyl)-6-{[4-(piperidin-1-ylmethyl)pyridin-2-yl]amino}-1,2-dihydro-2,7-naphthyridin-1-one;
8-(tert-butylamino)-2-(2-hydroxyethyl)-6-({4-[(oxetan-3-ylamino)methyl]pyridin-2-yl}amino)-1,2-dihydro-2,7-naphthyridin-1-one;
8-(tert-butylamino)-2-(2-hydroxyethyl)-6-[(5-methoxypyridin-2-yl)amino]-1,2-dihydro-2,7-naphthyridin-1-one;
8-(tert-butylamino)-2-(2-hydroxyethyl)-6-{[6-(hydroxymethyl)pyrimidin-4-yl]amino}-1,2-dihydro-2,7-naphthyridin-1-one;
8-(tert-butylamino)-2-(2-hydroxyethyl)-6-[(4-{[(2-hydroxyethyl)amino]methyl}pyridin-2-yl)amino]-1,2-dihydro-2,7-naphthyridin-1-one;
8-(tert-butylamino)-2-(2-hydroxyethyl)-6-{[4-(3-hydroxyoxetan-3-yl)pyridin-2-yl]amino}-1,2-dihydro-2,7-naphthyridin-1-one;
8-(tert-butylamino)-6-({5-fluoro-4-[(3-hydroxy-3-methylazetidin-1-yl)methyl]pyridin-2-yl}amino)-2-(2-hydroxyethyl)-1,2-dihydro-2,7-naphthyridin-1-one;
8-(tert-butylamino)-6-({5-fluoro-4-[(3-hydroxyazetidin-1-yl)methyl]pyridin-2-yl}amino)-2-(2-hydroxyethyl)-1,2-dihydro-2,7-naphthyridin-1-one;
6-{[4-(2-amino-1-hydroxyethyl)pyridin-2-yl]amino}-8-(tert-butylamino)-2-(2-hydroxyethyl)-1,2-dihydro-2,7-naphthyridin-1-one;
2-[8-(tert-butylamino)-1-oxo-6-{[4-(2,2,2-trifluoro-1-hydroxyethyl)pyridin-2-yl]amino}-1,2-dihydro-2,7-naphthyridin-2-yl]acetamide;
2-{8-[(1-methylcyclopropyl)amino]-1-oxo-6-{[4-(2,2,2-trifluoro-1-hydroxyethyl)pyridin-2-yl]amino}-1,2-dihydro-2,7-naphthyridin-2-yl}acetamide;
8-(tert-butylamino)-6-{[4-(difluoromethyl)pyridin-2-yl]amino}-2-(2-hydroxyethyl)-1,2-dihydro-2,7-naphthyridin-1-one;
8-(tert-butylamino)-6-[(4-tert-butylpyridin-2-yl)amino]-2-(2-hydroxyethyl)-1,2-dihydro-2,7-naphthyridin-1-one;
2-(6-{[4-(3-hydroxyoxetan-3-yl)pyridin-2-yl]amino}-8-[(1-methylcyclopropyl)amino]-1-oxo-1,2-dihydro-2,7-naphthyridin-2-yl)acetamide;
8-(tert-butylamino)-6-[(6-ethenylpyrimidin-4-yl)amino]-2-(2-hydroxyethyl)-1,2-dihydro-2,7-naphthyridin-1-one;
8-(tert-butylamino)-6-[(6-ethylpyrimidin-4-yl)amino]-2-(2-hydroxyethyl)-1,2-dihydro-2,7-naphthyridin-1-one;
2-(6-{[6-(2-hydroxypropan-2-yl)pyrimidin-4-yl]amino}-8-[(1-methylcyclopropyl)amino]-1-oxo-1,2-dihydro-2,7-naphthyridin-2-yl)acetamide;
8-(tert-butylamino)-2-(2-hydroxyethyl)-6-({6-[(1E)-3-methoxyprop-1-en-1-yl]pyrimidin-4-yl}amino)-1,2-dihydro-2,7-naphthyridin-1-one;
8-(tert-butylamino)-2-(2-hydroxyethyl)-6-{[6-(3-methoxypropyl)pyrimidin-4-yl]amino}-1,2-dihydro-2,7-naphthyridin-1-one;
8-(tert-butylamino)-6-{[6-(cyclohex-1-en-1-yl)pyrimidin-4-yl]amino}-2-(2-hydroxyethyl)-1,2-dihydro-2,7-naphthyridin-1-one;
8-(tert-butylamino)-6-{[6-(3,4-dihydro-2H-pyran-6-yl)pyrimidin-4-yl]amino}-2-(2-hydroxyethyl)-1,2-dihydro-2,7-naphthyridin-1-one;
8-(tert-butylamino)-6-[(6-cyclohexylpyrimidin-4-yl)amino]-2-(2-hydroxyethyl)-1,2-dihydro-2,7-naphthyridin-1-one;
8-(tert-butylamino)-2-(2-hydroxyethyl)-6-{[6-(oxan-2-yl)pyrimidin-4-yl]amino}-1,2-dihydro-2,7-naphthyridin-1-one;
8-(tert-butylamino)-6-({6-[(1E)-4-hydroxybut-1-en-1-yl]pyrimidin-4-yl}amino)-2-(2-hydroxyethyl)-1,2-dihydro-2,7-naphthyridin-1-one;
8-(tert-butylamino)-2-(2-hydroxyethyl)-6-{[4-(1,1,1-trifluoro-2-hydroxypropan-2-yl)pyridin-2-yl]amino}-1,2-dihydro-2,7-naphthyridin-1-one;
8-(tert-butylamino)-6-{[6-(4-hydroxybutyl)pyrimidin-4-yl]amino}-2-(2-hydroxyethyl)-1,2-dihydro-2,7-naphthyridin-1-one;
8-(tert-butylamino)-6-{[6-(3-hydroxy-3-methylazetidin-1-yl)pyrimidin-4-yl]amino}-2-(2-hydroxyethyl)-1,2-dihydro-2,7-naphthyridin-1-one;
8-(tert-butylamino)-6-{[6-(3-hydroxyazetidin-1-yl)pyrimidin-4-yl]amino}-2-(2-hydroxyethyl)-1,2-dihydro-2,7-naphthyridin-1-one;
8-(tert-butylamino)-2-(2-hydroxyethyl)-6-{[6-(4-oxopiperidin-1-yl)pyrimidin-4-yl]amino}-1,2-dihydro-2,7-naphthyridin-1-one;
6-[(6-acetylpyrimidin-4-yl)amino]-8-(tert-butylamino)-2-(2-hydroxyethyl)-1,2-dihydro-2,7-naphthyridin-1-one;
8-(tert-butylamino)-2-(2-hydroxyethyl)-6-{[6-(4-hydroxypiperidin-1-yl)pyrimidin-4-yl]amino}-1,2-dihydro-2,7-naphthyridin-1-one;
8-(tert-butylamino)-2-(2-hydroxyethyl)-6-({6-[3-(trifluoromethyl)-1H-pyrazol-4-yl]pyrimidin-4-yl}amino)-1,2-dihydro-2,7-naphthyridin-1-one;
8-(tert-butylamino)-2-(2-hydroxyethyl)-6-{[6-(1-methyl-1H-pyrazol-4-yl)pyrimidin-4-yl]amino}-1,2-dihydro-2,7-naphthyridin-1-one;
8-(tert-butylamino)-2-(2-hydroxyethyl)-6-{[6-(2-methoxypyrimidin-5-yl)pyrimidin-4-yl]amino}-1,2-dihydro-2,7-naphthyridin-1-one;
8-(tert-butylamino)-2-(2-hydroxyethyl)-6-{[6-(piperidin-4-yl)pyrimidin-4-yl]amino}-1,2-dihydro-2,7-naphthyridin-1-one;
8-(tert-butylamino)-2-(2-hydroxyethyl)-6-{[6-(1H-pyrazol-4-yl)pyrimidin-4-yl]amino}-1,2-dihydro-2,7-naphthyridin-1-one;
8-(tert-butylamino)-2-(2-hydroxyethyl)-6-({6-[1-(2-hydroxyethyl)piperidin-4-yl]pyrimidin-4-yl}amino)-1,2-dihydro-2,7-naphthyridin-1-one;
8-(tert-butylamino)-6-{[6-(2-hydroxy-2-methylpropoxy)pyrimidin-4-yl]amino}-2-(2-hydroxyethyl)-1,2-dihydro-2,7-naphthyridin-1-one;
8-(tert-butylamino)-2-[(2S)-2,3-dihydroxypropyl]-6-{[6-(2-hydroxyethoxy)pyrimidin-4-yl]amino}-1,2-dihydro-2,7-naphthyridin-1-one;
8-(tert-butylamino)-2-[(2S)-2,3-dihydroxypropyl]-6-[(6-{[(2S)-1-hydroxypropan-2-yl]oxy}pyrimidin-4-yl)amino]-1,2-dihydro-2,7-naphthyridin-1-one;
8-(tert-butylamino)-2-[(2S)-2,3-dihydroxypropyl]-6-{[6-(piperidin-4-yloxy)pyrimidin-4-yl]amino}-1,2-dihydro-2,7-naphthyridin-1-one;
8-(tert-butylamino)-2-(2-hydroxyethyl)-6-{[6-(piperidin-4-yloxy)pyrimidin-4-yl]amino}-1,2-dihydro-2,7-naphthyridin-1-one;
8-(tert-butylamino)-2-(2-hydroxyethyl)-6-[(6-{[1-(2-hydroxyethyl)piperidin-4-yl]oxy}pyrimidin-4-yl)amino]-1,2-dihydro-2,7-naphthyridin-1-one;
8-(tert-butylamino)-2-(2-hydroxyethyl)-6-[(6-{[1-(2-methoxyethyl)piperidin-4-yl]oxy}pyrimidin-4-yl)amino]-1,2-dihydro-2,7-naphthyridin-1-one;
8-(tert-butylamino)-2-(2-hydroxyethyl)-6-({6-[(1-methylpiperidin-4-yl)oxy]pyrimidin-4-yl}amino)-1,2-dihydro-2,7-naphthyridin-1-one;
8-(tert-butylamino)-2-[(2S)-2,3-dihydroxypropyl]-6-({6-[(1-methylpiperidin-4-yl)oxy]pyrimidin-4-yl}amino)-1,2-dihydro-2,7-naphthyridin-1-one;
2-(2-hydroxyethyl)-8-({3-methyl-4-[(morpholin-4-yl)carbonyl]phenyl}amino)-6-[(4-methylpyridin-2-yl)amino]-1,2-dihydro-2,7-naphthyridin-1-one;
N-(3-methoxypropyl)-N-methyl-4-({7-methyl-3-[(4-methylpyridin-2-yl)amino]-8-oxo-7,8-dihydro-2,7-naphthyridin-1-yl}amino)benzene-1-sulfonamide;
N-(2-methoxyethyl)-4-({7-methyl-3-[(4-methylpyridin-2-yl)amino]-8-oxo-7,8-dihydro-2,7-naphthyridin-1-yl}amino)benzamide;
2-methyl-6-[(4-methylpyridin-2-yl)amino]-8-({4-[2-(morpholin-4-yl)ethoxy]phenyl}amino)-1,2-dihydro-2,7-naphthyridin-1-one;
2-methyl-5-({7-methyl-3-[(4-methylpyridin-2-yl)amino]-8-oxo-7,8-dihydro-2,7-naphthyridin-1-yl}amino)benzene-1-sulfonamide;
2-(2-hydroxyethyl)-8-[(2-methyl-2H-indazol-6-yl)amino]-6-(pyrazin-2-ylamino)-1,2-dihydro-2,7-naphthyridin-1-one;
8-[(2,3-dimethyl-2H-indazol-6-yl)amino]-2-(2-hydroxyethyl)-6-(pyrazin-2-ylamino)-1,2-dihydro-2,7-naphthyridin-1-one;
6-[(4-hydroxycyclohexyl)amino]-8-{[4-(morpholin-4-yl)phenyl]amino}-1,2-dihydro-2,7-naphthyridin-1-one;
3-{[7-(2-hydroxyethyl)-1-[(2-methyl-2H-indazol-5-yl)amino]-8-oxo-7,8-dihydro-2,7-naphthyridin-3-yl]amino}pyrazin-1-ium-1-olate;
2-(2-hydroxyethyl)-8-[(2-methyl-2H-indazol-5-yl)amino]-6-(pyrazin-2-ylamino)-1,2-dihydro-2,7-naphthyridin-1-one;
2-[8-(tert-butylamino)-1-oxo-6-(pyrazin-2-ylamino)-1,2-dihydro-2,7-naphthyridin-2-yl]propanamide;
3-[8-(tert-butylamino)-1-oxo-6-(pyrazin-2-ylamino)-1,2-dihydro-2,7-naphthyridin-2-yl]propanamide;
2-[8-(tert-butylamino)-1-oxo-6-(pyrazin-2-ylamino)-1,2-dihydro-2,7-naphthyridin-2-yl]acetamide;
2-[6-(2-aminopyrimidin-5-yl)-8-(tert-butylamino)-1-oxo-1,2-dihydro-2,7-naphthyridin-2-yl]acetamide;
8-(tert-butylamino)-2-(2,3-dihydroxypropyl)-6-(pyrazin-2-ylamino)-1,2-dihydro-2,7-naphthyridin-1-one;
2-[6-(2-aminopyrimidin-5-yl)-8-(tert-butylamino)-1-oxo-1,2-dihydro-2,7-naphthyridin-2-yl]propanamide;
6-(2-aminopyrimidin-5-yl)-8-(tert-butylamino)-4-chloro-2-(2-hydroxyethyl)-1,2-dihydro-2,7-naphthyridin-1-one;
2-[6-(2-aminopyrimidin-5-yl)-8-(tert-butylamino)-1-oxo-1,2-dihydro-2,7-naphthyridin-2-yl]acetic acid;
8-(tert-butylamino)-2-[(2S)-2,3-dihydroxypropyl]-6-(pyrazin-2-ylamino)-1,2-dihydro-2,7-naphthyridin-1-one;
8-(tert-butylamino)-2-[(2R)-2,3-dihydroxypropyl]-6-(pyrazin-2-ylamino)-1,2-dihydro-2,7-naphthyridin-1-one;
8-[(2-cyclopropylpropan-2-yl)amino]-2-(2-hydroxyethyl)-6-(pyrazin-2-ylamino)-1,2-dihydro-2,7-naphthyridin-1-one;
6-(2-aminopyrimidin-5-yl)-8-(tert-butylamino)-2-(2-hydroxyethyl)-4-methyl-1,2-dihydro-2,7-naphthyridin-1-one;
2-[8-(tert-butylamino)-1-oxo-6-(pyrazin-2-ylamino)-1,2-dihydro-2,7-naphthyridin-2-yl]-2-methylpropanamide;
2-[6-(2-aminopyrimidin-5-yl)-8-(tert-butylamino)-1-oxo-1,2-dihydro-2,7-naphthyridin-2-yl]-2-methylpropanamide;
3-{[1-(tert-butylamino)-7-[(2S)-2,3-dihydroxypropyl]-8-oxo-7,8-dihydro-2,7-naphthyridin-3-yl]amino}pyrazin-1-ium-1-olate;
3-{[1-(tert-butylamino)-7-[(2R)-2,3-dihydroxypropyl]-8-oxo-7,8-dihydro-2,7-naphthyridin-3-yl]amino}pyrazin-1-ium-1-olate;
8-(tert-butylamino)-2-[(2S)-2-hydroxy-2-phenylethyl]-6-(pyrazin-2-ylamino)-1,2-dihydro-2,7-naphthyridin-1-one;
8-(tert-butylamino)-2-(2-hydroxy-3-methoxypropyl)-6-(pyrazin-2-ylamino)-1,2-dihydro-2,7-naphthyridin-1-one;
6-(2-aminopyrimidin-5-yl)-8-(tert-butylamino)-2-[2-(morpholin-4-yl)-2-oxoethyl]-1,2-dihydro-2,7-naphthyridin-1-one;
8-(tert-butylamino)-6-(pyrazin-2-ylamino)-1,2-dihydro-2,7-naphthyridin-1-one;
2-{[1-(tert-butylamino)-7-(2-hydroxyethyl)-8-oxo-7,8-dihydro-2,7-naphthyridin-3-yl]amino}pyridine-4-carboxylic acid;
2-{[1-(tert-butylamino)-7-(2-hydroxyethyl)-8-oxo-7,8-dihydro-2,7-naphthyridin-3-yl]amino}pyridine-4-carboxamide;
2-{[1-(tert-butylamino)-7-(2-hydroxyethyl)-8-oxo-7,8-dihydro-2,7-naphthyridin-3-yl]amino}-N-(2-methoxyethyl)pyridine-4-carboxamide;
2-{[1-(tert-butylamino)-7-(2-hydroxyethyl)-8-oxo-7,8-dihydro-2,7-naphthyridin-3-yl]amino}-N-[2-(dimethylamino)ethyl]pyridine-4-carboxamide;
6-[(4-acetylpyridin-2-yl)amino]-8-(tert-butylamino)-2-(2-hydroxyethyl)-1,2-dihydro-2,7-naphthyridin-1-one;
8-(tert-butylamino)-2-(2-hydroxyethyl)-6-({4-[(1E)-1-(hydroxyimino)ethyl]pyridin-2-yl}amino)-1,2-dihydro-2,7-naphthyridin-1-one;
8-(tert-butylamino)-6-({4-[(1E)-1-{[2-(dimethylamino)ethoxy]imino}ethyl]pyridin-2-yl}amino)-2-(2-hydroxyethyl)-1,2-dihydro-2,7-naphthyridin-1-one;
2-{[1-(tert-butylamino)-7-(2-hydroxyethyl)-8-oxo-7,8-dihydro-2,7-naphthyridin-3-yl]amino}-N-[2-(1H-imidazol-4-yl)ethyl]pyridine-4-carboxamide;
8-(tert-butylamino)-2-(2-hydroxyethyl)-6-[(4-{[3-(hydroxymethyl)piperidin-1-yl]carbonyl}pyridin-2-yl)amino]-1,2-dihydro-2,7-naphthyridin-1-one;
8-(tert-butylamino)-2-(2-hydroxyethyl)-6-{[4-(1,2-oxazol-5-yl)pyridin-2-yl]amino}-1,2-dihydro-2,7-naphthyridin-1-one;
8-(tert-butylamino)-2-(2-hydroxyethyl)-6-{[4-(1H-pyrazol-3-yl)pyridin-2-yl]amino}-1,2-dihydro-2,7-naphthyridin-1-one;
6-{[4-(2-aminopyrimidin-4-yl)pyridin-2-yl]amino}-8-(tert-butylamino)-2-(2-hydroxyethyl)-1,2-dihydro-2,7-naphthyridin-1-one;
8-(tert-butylamino)-2-(2-hydroxyethyl)-6-(1,3-thiazol-2-ylamino)-1,2-dihydro-2,7-naphthyridin-1-one;
8-(tert-butylamino)-2-(2-hydroxyethyl)-6-{[4-(hydroxymethyl)pyridin-2-yl]amino}-1,2-dihydro-2,7-naphthyridin-1-one;
8-(tert-butylamino)-2-(2-hydroxyethyl)-6-[(4-{[(2-methoxyethyl)amino]methyl}pyridin-2-yl)amino]-1,2-dihydro-2,7-naphthyridin-1-one;
8-(tert-butylamino)-2-(2-hydroxyethyl)-6-({4-[(1E)-1-(methoxyimino)ethyl]pyridin-2-yl}amino)-1,2-dihydro-2,7-naphthyridin-1-one;
8-(tert-butylamino)-6-[(5-fluoropyridin-2-yl)amino]-2-(2-hydroxyethyl)-1,2-dihydro-2,7-naphthyridin-1-one;
6-{[1-(tert-butylamino)-7-(2-hydroxyethyl)-8-oxo-7,8-dihydro-2,7-naphthyridin-3-yl]amino}pyridine-3-carbonitrile;
8-(tert-butylamino)-2-(2-hydroxyethyl)-6-{[4-(2-hydroxypropan-2-yl)pyridin-2-yl]amino}-1,2-dihydro-2,7-naphthyridin-1-one;
8-(tert-butylamino)-2-(2-hydroxyethyl)-6-[(4-methanesulfonylpyridin-2-yl)amino]-1,2-dihydro-2,7-naphthyridin-1-one;
8-(tert-butylamino)-2-(2-hydroxyethyl)-6-{[4-(1-hydroxyethyl)pyridin-2-yl]amino}-1,2-dihydro-2,7-naphthyridin-1-one;
2-ethyl-8-[(1-hydroxy-2-methylpropan-2-yl)amino]-6-{[4-(2-hydroxypropan-2-yl)pyridin-2-yl]amino}-1,2-dihydro-2,7-naphthyridin-1-one;
2-ethyl-6-{[4-(2-hydroxypropan-2-yl)pyridin-2-yl]amino}-8-[(1-methylcyclobutyl)amino]-1,2-dihydro-2,7-naphthyridin-1-one;
2-ethyl-8-{[1-(hydroxymethyl)cyclobutyl]amino}-6-{[4-(2-hydroxypropan-2-yl)pyridin-2-yl]amino}-1,2-dihydro-2,7-naphthyridin-1-one;
2-(2-hydroxyethyl)-6-{[4-(2-hydroxypropan-2-yl)pyridin-2-yl]amino}-8-[(1-methylcyclobutyl)amino]-1,2-dihydro-2,7-naphthyridin-1-one;
2-(2-hydroxyethyl)-6-{[4-(2-hydroxypropan-2-yl)pyridin-2-yl]amino}-8-(propan-2-ylamino)-1,2-dihydro-2,7-naphthyridin-1-one;
8-[(4-hydroxycyclohexyl)amino]-6-{[4-(2-hydroxypropan-2-yl)pyridin-2-yl]amino}-2-(propan-2-yl)-1,2-dihydro-2,7-naphthyridin-1-one;
6-{[4-(aminomethyl)pyridin-2-yl]amino}-8-(tert-butylamino)-2-(2-hydroxyethyl)-1,2-dihydro-2,7-naphthyridin-1-one;
8-(tert-butylamino)-2-(2-hydroxyethyl)-6-{[4-(3-hydroxypentan-3-yl)pyridin-2-yl]amino}-1,2-dihydro-2,7-naphthyridin-1-one;
8-(tert-butylamino)-2-(2-hydroxyethyl)-6-{[4-(methoxymethyl)pyridin-2-yl]amino}-1,2-dihydro-2,7-naphthyridin-1-one;
8-(tert-butylamino)-2-(2-hydroxyethyl)-6-{[4-(1-hydroxypropyl)pyridin-2-yl]amino}-1,2-dihydro-2,7-naphthyridin-1-one;
6-{[4-(2-aminopropan-2-yl)pyridin-2-yl]amino}-8-(tert-butylamino)-2-(2-hydroxyethyl)-1,2-dihydro-2,7-naphthyridin-1-one;
6-{[4-(1-aminoethyl)pyridin-2-yl]amino}-8-(tert-butylamino)-2-(2-hydroxyethyl)-1,2-dihydro-2,7-naphthyridin-1-one;
8-(tert-butylamino)-2-(2-hydroxyethyl)-6-({4-[(1R)-1-hydroxyethyl]pyridin-2-yl}amino)-1,2-dihydro-2,7-naphthyridin-1-one;
8-(tert-butylamino)-2-(2-hydroxyethyl)-6-({4-[(1S)-1-hydroxyethyl]pyridin-2-yl}amino)-1,2-dihydro-2,7-naphthyridin-1-one;
8-(tert-butylamino)-2-(2-hydroxyethyl)-6-{[4-(1-hydroxyethyl)pyrimidin-2-yl]amino}-1,2-dihydro-2,7-naphthyridin-1-one;
8-(tert-butylamino)-2-(2-hydroxyethyl)-6-{[6-(2-hydroxypropan-2-yl)pyrimidin-4-yl]amino}-1,2-dihydro-2,7-naphthyridin-1-one;
8-(tert-butylamino)-6-{[5-fluoro-4-(2-hydroxypropan-2-yl)pyridin-2-yl]amino}-2-(2-hydroxyethyl)-1,2-dihydro-2,7-naphthyridin-1-one;
2-(2-hydroxyethyl)-6-{[4-(1-hydroxyethyl)pyridin-2-yl]amino}-8-[(1-methylcyclobutyl)amino]-1,2-dihydro-2,7-naphthyridin-1-one;
8-(tert-butylamino)-2-(2-hydroxyethyl)-6-{[4-(1-hydroxyethyl)pyridin-2-yl]amino}-1,2-dihydro-2,7-naphthyridin-1-one;
6-[6-amino-5-(hydroxymethyl)pyridin-3-yl]-8-(tert-butylamino)-2-(2-hydroxyethyl)-1,2-dihydro-2,7-naphthyridin-1-one;
2-(6-{[4-(1-hydroxyethyl)pyridin-2-yl]amino}-8-[(1-methylcyclobutyl)amino]-1-oxo-1,2-dihydro-2,7-naphthyridin-2-yl)acetamide;
2-(6-{[4-(2-hydroxypropan-2-yl)pyridin-2-yl]amino}-8-[(1-methylcyclobutyl)amino]-1-oxo-1,2-dihydro-2,7-naphthyridin-2-yl)acetamide;
2-(6-{[4-(1-hydroxyethyl)pyridin-2-yl]amino}-8-[(1-methylcyclopropyl)amino]-1-oxo-1,2-dihydro-2,7-naphthyridin-2-yl)acetamide;
2-(6-{[4-(2-hydroxypropan-2-yl)pyridin-2-yl]amino}-8-[(1-methylcyclopropyl)amino]-1-oxo-1,2-dihydro-2,7-naphthyridin-2-yl)acetamide;
2-[8-(tert-butylamino)-6-{[4-(1-hydroxyethyl)pyridin-2-yl]amino}-1-oxo-1,2-dihydro-2,7-naphthyridin-2-yl]acetamide;
2-[8-(tert-butylamino)-6-{[4-(2-hydroxypropan-2-yl)pyridin-2-yl]amino}-1-oxo-1,2-dihydro-2,7-naphthyridin-2-yl]acetamide;
8-(tert-butylamino)-2-(2-hydroxyethyl)-6-{[6-(1-hydroxyethyl)pyrimidin-4-yl]amino}-1,2-dihydro-2,7-naphthyridin-1-one;
8-(tert-butylamino)-6-{[5-chloro-4-(2-hydroxypropan-2-yl)pyridin-2-yl]amino}-2-(2-hydroxyethyl)-1,2-dihydro-2,7-naphthyridin-1-one;
2-(2,3-dihydroxypropyl)-6-{[6-(2-hydroxypropan-2-yl)pyrimidin-4-yl]amino}-8-[(1-methylcyclopropyl)amino]-1,2-dihydro-2,7-naphthyridin-1-one;
8-(tert-butylamino)-2-(2,3-dihydroxypropyl)-6-{[6-(2-hydroxypropan-2-yl)pyrimidin-4-yl]amino}-1,2-dihydro-2,7-naphthyridin-1-one;
8-(tert-butylamino)-2-(2-hydroxy-2-methylpropyl)-6-{[6-(2-hydroxypropan-2-yl)pyrimidin-4-yl]amino}-1,2-dihydro-2,7-naphthyridin-1-one;
2-(2,3-dihydroxypropyl)-6-{[6-(2-hydroxypropan-2-yl)pyrimidin-4-yl]amino}-8-[(1-methylcyclobutyl)amino]-1,2-dihydro-2,7-naphthyridin-1-one;
2-(2-hydroxy-2-methylpropyl)-6-{[6-(2-hydroxypropan-2-yl)pyrimidin-4-yl]amino}-8-[(1-methylcyclobutyl)amino]-1,2-dihydro-2,7-naphthyridin-1-one;
8-(tert-butylamino)-6-{[6-(2-hydroxypropan-2-yl)pyrimidin-4-yl]amino}-2-(oxetan-3-yl)-1,2-dihydro-2,7-naphthyridin-1-one;
8-(tert-butylamino)-2-(2-hydroxyethyl)-6-[(6-methoxypyrimidin-4-yl)amino]-1,2-dihydro-2,7-naphthyridin-1-one;
8-(tert-butylamino)-2-(2-hydroxyethyl)-6-(pyrimidin-4-ylamino)-1,2-dihydro-2,7-naphthyridin-1-one;
6-(2-aminopyrimidin-5-yl)-8-(tert-butylamino)-2-(2-methanesulfonylethyl)-1,2-dihydro-2,7-naphthyridin-1-one;
8-(tert-butylamino)-2-(2-methanesulfonylethyl)-6-(pyrazin-2-ylamino)-1,2-dihydro-2,7-naphthyridin-1-one;
8-(tert-butylamino)-6-{[6-(2-hydroxypropan-2-yl)pyrimidin-4-yl]amino}-2-(2-methanesulfonylethyl)-1,2-dihydro-2,7-naphthyridin-1-one;
8-(tert-butylamino)-2-[(2S)-2,3-dihydroxypropyl]-6-{[6-(2-hydroxypropan-2-yl)pyrimidin-4-yl]amino}-1,2-dihydro-2,7-naphthyridin-1-one;
8-(tert-butylamino)-2-(1,3-dihydroxypropan-2-yl)-6-{[6-(2-hydroxypropan-2-yl)pyrimidin-4-yl]amino}-1,2-dihydro-2,7-naphthyridin-1-one;
6-(2-aminopyrimidin-5-yl)-8-(oxan-4-ylamino)-1,2-dihydro-2,7-naphthyridin-1-one;
8-amino-6-(2-aminopyrimidin-5-yl)-2-ethyl-1,2-dihydro-2,7-naphthyridin-1-one;
N-[3-(2-aminopyrimidin-5-yl)-7-ethyl-8-oxo-7,8-dihydro-2,7-naphthyridin-1-yl]methanesulfonamide;
N-[7-ethyl-8-oxo-3-(pyrazin-2-ylamino)-7,8-dihydro-2,7-naphthyridin-1-yl]methanesulfonamide;
2-[6-(2-aminopyrimidin-5-yl)-8-{[3-(hydroxymethyl)cyclobutyl]amino}-1-oxo-1,2-dihydro-2,7-naphthyridin-2-yl]acetamide;
2-(8-{[3-(hydroxymethyl)cyclobutyl]amino}-1-oxo-6-(pyrazin-2-ylamino)-1,2-dihydro-2,7-naphthyridin-2-yl)acetamide;
6-(2-aminopyrimidin-5-yl)-2-ethyl-8-{[3-(hydroxymethyl)cyclobutyl]amino}-1,2-dihydro-2,7-naphthyridin-1-one;
6-(2-aminopyrimidin-5-yl)-2-ethyl-8-{[(1R)-3-fluorocyclopentyl]amino}-1,2-dihydro-2,7-naphthyridin-1-one;
2-[6-(2-aminopyrimidin-5-yl)-8-(oxan-4-ylamino)-1-oxo-1,2-dihydro-2,7-naphthyridin-2-yl]acetamide;
2-[6-(2-aminopyrimidin-5-yl)-8-(oxan-4-ylamino)-1-oxo-1,2-dihydro-2,7-naphthyridin-2-yl]acetonitrile;
2-[8-(cyclopentylamino)-1-oxo-6-(pyrazin-2-ylamino)-1,2-dihydro-2,7-naphthyridin-2-yl]acetamide;
2-[8-(cyclopentylamino)-1-oxo-6-(pyrazin-2-ylamino)-1,2-dihydro-2,7-naphthyridin-2-yl]acetonitrile;
2-[8-(cyclobutylamino)-1-oxo-6-(pyrazin-2-ylamino)-1,2-dihydro-2,7-naphthyridin-2-yl]acetamide;
8-(cyclobutylamino)-2-[(3-methyloxetan-3-yl)methyl]-6-(pyrazin-2-ylamino)-1,2-dihydro-2,7-naphthyridin-1-one;
2-[6-(2-aminopyrimidin-5-yl)-8-(cyclopentylamino)-1-oxo-1,2-dihydro-2,7-naphthyridin-2-yl]acetonitrile;
2-[6-(2-aminopyrimidin-5-yl)-8-(cyclopentylamino)-1-oxo-1,2-dihydro-2,7-naphthyridin-2-yl]acetamide;
6-(2-aminopyrimidin-5-yl)-8-[(3,3-difluorocyclobutyl)amino]-2-[(3-methyloxetan-3-yl)methyl]-1,2-dihydro-2,7-naphthyridin-1-one;
2-[6-(2-aminopyrimidin-5-yl)-8-[(3,3-difluorocyclobutyl)amino]-1-oxo-1,2-dihydro-2,7-naphthyridin-2-yl]acetamide;
6-(2-aminopyrimidin-5-yl)-8-(cyclopentylamino)-2-[(3-methyloxetan-3-yl)methyl]-1,2-dihydro-2,7-naphthyridin-1-one;
6-(2-aminopyrimidin-5-yl)-8-(cyclopentylamino)-2-{[3-(hydroxymethyl)oxetan-3-yl]methyl}-1,2-dihydro-2,7-naphthyridin-1-one;
8-(cyclopentylamino)-2-[(3-methyloxetan-3-yl)methyl]-6-(pyrazin-2-ylamino)-1,2-dihydro-2,7-naphthyridin-1-one;
8-(cyclopentylamino)-2-{[3-(hydroxymethyl)oxetan-3-yl]methyl}-6-(pyrazin-2-ylamino)-1,2-dihydro-2,7-naphthyridin-1-one;
8-[(3,3-difluorocyclobutyl)amino]-6-(pyrazin-2-ylamino)-1,2-dihydro-2,7-naphthyridin-1-one;
8-[(3,3-difluorocyclobutyl)amino]-2-[(3-methyloxetan-3-yl)methyl]-6-(pyrazin-2-ylamino)-1,2-dihydro-2,7-naphthyridin-1-one;
8-(cyclopentylamino)-6-(pyrazin-2-ylamino)-1,2-dihydro-2,7-naphthyridin-1-one;
8-(cyclobutylamino)-6-(pyrazin-2-ylamino)-1,2-dihydro-2,7-naphthyridin-1-one;
2-[(3-methyloxetan-3-yl)methyl]-8-(oxan-4-ylamino)-6-(pyrazin-2-ylamino)-1,2-dihydro-2,7-naphthyridin-1-one;
6-(2-aminopyrimidin-5-yl)-2-[(3-methyloxetan-3-yl)methyl]-8-(oxan-4-ylamino)-1,2-dihydro-2,7-naphthyridin-1-one;
6-(2-aminopyrimidin-5-yl)-8-(oxan-4-ylamino)-2-(oxetan-3-yl)-1,2-dihydro-2,7-naphthyridin-1-one;
6-(2-aminopyrimidin-5-yl)-8-(cyclobutylamino)-2-[(3-methyloxetan-3-yl)methyl]-1,2-dihydro-2,7-naphthyridin-1-one;
6-(2-aminopyrimidin-5-yl)-8-(cyclobutylamino)-2-(oxetan-3-yl)-1,2-dihydro-2,7-naphthyridin-1-one;
6-(2-aminopyrimidin-5-yl)-2-[(3-methyloxetan-3-yl)methyl]-8-(oxetan-3-ylamino)-1,2-dihydro-2,7-naphthyridin-1-one;
6-(2-aminopyrimidin-5-yl)-8-(cyclobutylamino)-2-[(2S)-2,3-dihydroxypropyl]-1,2-dihydro-2,7-naphthyridin-1-one;
8-(cyclopentylamino)-2-[(2S)-2,3-dihydroxypropyl]-6-(pyrazin-2-ylamino)-1,2-dihydro-2,7-naphthyridin-1-one;
6-(2-aminopyrimidin-5-yl)-8-(cyclopentylamino)-2-[(2S)-2,3-dihydroxypropyl]-1,2-dihydro-2,7-naphthyridin-1-one;
6-[(5-chloro-4-methylpyridin-2-yl)amino]-8-[(1-hydroxy-2-methylpropan-2-yl)amino]-2-methyl-1,2-dihydro-2,7-naphthyridin-1-one;
2-ethyl-6-[(4-methylpyridin-2-yl)amino]-8-[(3,3,3-trifluoro-2-hydroxypropyl)amino]-1,2-dihydro-2,7-naphthyridin-1-one;
2-ethyl-6-(pyrazin-2-ylamino)-8-[(3,3,3-trifluoro-2-hydroxypropyl)amino]-1,2-dihydro-2,7-naphthyridin-1-one;
2-ethyl-6-[(4-methylpyridin-2-yl)amino]-8-[(3,3,3-trifluoropropyl)amino]-1,2-dihydro-2,7-naphthyridin-1-one;
2-ethyl-6-(pyrazin-2-ylamino)-8-[(3,3,3-trifluoropropyl)amino]-1,2-dihydro-2,7-naphthyridin-1-one;
2-ethyl-8-[(4-hydroxy-2-methylbutan-2-yl)amino]-6-[(4-methylpyridin-2-yl)amino]-1,2-dihydro-2,7-naphthyridin-1-one;
2-ethyl-8-[(4-hydroxy-2-methylbutan-2-yl)amino]-6-(pyrazin-2-ylamino)-1,2-dihydro-2,7-naphthyridin-1-one;
4-chloro-8-[(4-hydroxycyclohexyl)amino]-2-methyl-6-[(4-methylpyridin-2-yl)amino]-1,2-dihydro-2,7-naphthyridin-1-one;
4-chloro-8-[(4-hydroxycyclohexyl)amino]-2-methyl-6-(pyrazin-2-ylamino)-1,2-dihydro-2,7-naphthyridin-1-one;
6-[(4-chloropyridin-2-yl)amino]-8-[(1-hydroxy-2-methylpropan-2-yl)amino]-2-methyl-1,2-dihydro-2,7-naphthyridin-1-one;
2-ethyl-8-{[1-(hydroxymethyl)cyclopentyl]amino}-6-[(4-methylpyridin-2-yl)amino]-1,2-dihydro-2,7-naphthyridin-1-one;
2-ethyl-8-{[1-(hydroxymethyl)cyclopentyl]amino}-6-(pyrazin-2-ylamino)-1,2-dihydro-2,7-naphthyridin-1-one;
2-ethyl-8-{[(1S,3S)-3-(hydroxymethyl)cyclopentyl]amino}-6-[(4-methylpyridin-2-yl)amino]-1,2-dihydro-2,7-naphthyridin-1-one;
2-ethyl-8-{[(1R,3R)-3-(hydroxymethyl)cyclopentyl]amino}-6-[(4-methylpyridin-2-yl)amino]-1,2-dihydro-2,7-naphthyridin-1-one;
2-ethyl-8-{[(1R,3R)-3-(hydroxymethyl)cyclopentyl]amino}-6-(pyrazin-2-ylamino)-1,2-dihydro-2,7-naphthyridin-1-one;
2-ethyl-8-{[(1S,3R)-3-(hydroxymethyl)cyclopentyl]amino}-6-[(4-methylpyridin-2-yl)amino]-1,2-dihydro-2,7-naphthyridin-1-one;
2-ethyl-8-{[(1S,3R)-3-(hydroxymethyl)cyclopentyl]amino}-6-(pyrazin-2-ylamino)-1,2-dihydro-2,7-naphthyridin-1-one;
2-ethyl-8-{[(1R,3S)-3-(hydroxymethyl)cyclopentyl]amino}-6-[(4-methylpyridin-2-yl)amino]-1,2-dihydro-2,7-naphthyridin-1-one;
2-ethyl-8-{[(1R,3S)-3-(hydroxymethyl)cyclopentyl]amino}-6-(pyrazin-2-ylamino)-1,2-dihydro-2,7-naphthyridin-1-one;
2-ethyl-8-{[1-(hydroxymethyl)cyclopropyl]amino}-6-[(4-methylpyridin-2-yl)amino]-1,2-dihydro-2,7-naphthyridin-1-one;
2-ethyl-8-{[1-(hydroxymethyl)cyclopropyl]amino}-6-(pyrazin-2-ylamino)-1,2-dihydro-2,7-naphthyridin-1-one;
8-[(4-hydroxycyclohexyl)amino]-6-[(4-hydroxypyrimidin-2-yl)amino]-2-methyl-1,2-dihydro-2,7-naphthyridin-1-one;
8-[(4-hydroxycyclohexyl)amino]-6-[(4-hydroxypyrimidin-2-yl)amino]-2-methyl-1,2-dihydro-2,7-naphthyridin-1-one;
8-(cyclobutylamino)-6-[(4-hydroxypyrimidin-2-yl)amino]-2-methyl-1,2-dihydro-2,7-naphthyridin-1-one;
8-amino-2-ethyl-6-[(4-methylpyridin-2-yl)amino]-1,2-dihydro-2,7-naphthyridin-1-one;
8-amino-2-ethyl-6-(pyrazin-2-ylamino)-1,2-dihydro-2,7-naphthyridin-1-one;
8-amino-2-ethyl-6-[(4-hydroxypyrimidin-2-yl)amino]-1,2-dihydro-2,7-naphthyridin-1-one;
4-({3-[(4-hydroxypyrimidin-2-yl)amino]-7-methyl-8-oxo-7,8-dihydro-2,7-naphthyridin-1-yl}amino)benzamide;
6-[(4-hydroxypyrimidin-2-yl)amino]-2-methyl-8-({4-[(morpholin-4-yl)carbonyl]phenyl}amino)-1,2-dihydro-2,7-naphthyridin-1-one;
2-ethyl-8-{[(2S)-1-hydroxy-3,3-dimethylbutan-2-yl]amino}-6-[(4-methylpyridin-2-yl)amino]-1,2-dihydro-2,7-naphthyridin-1-one;
2-ethyl-8-{[(2S)-1-hydroxy-3,3-dimethylbutan-2-yl]amino}-6-(pyrazin-2-ylamino)-1,2-dihydro-2,7-naphthyridin-1-one;
8-[(1,3-dihydroxy-2-methylpropan-2-yl)amino]-2-ethyl-6-[(4-methylpyridin-2-yl)amino]-1,2-dihydro-2,7-naphthyridin-1-one;
8-[(1,3-dihydroxy-2-methylpropan-2-yl)amino]-2-ethyl-6-(pyrazin-2-ylamino)-1,2-dihydro-2,7-naphthyridin-1-one;
2-ethyl-8-{[1-(hydroxymethyl)cyclobutyl]amino}-6-[(4-methylpyridin-2-yl)amino]-1,2-dihydro-2,7-naphthyridin-1-one;
8-amino-2-(2-hydroxyethyl)-6-(pyrazin-2-ylamino)-1,2-dihydro-2,7-naphthyridin-1-one;
8-amino-2-(2-hydroxyethyl)-6-[(4-hydroxypyrimidin-2-yl)amino]-1,2-dihydro-2,7-naphthyridin-1-one;
8-amino-2-(2-hydroxyethyl)-6-[(4-methylpyridin-2-yl)amino]-1,2-dihydro-2,7-naphthyridin-1-one;
8-[(2,2-dimethylpropyl)amino]-2-(2-hydroxyethyl)-6-[(4-methylpyridin-2-yl)amino]-1,2-dihydro-2,7-naphthyridin-1-one;
8-[(2,2-difluoroethyl)amino]-2-(2-hydroxyethyl)-6-[(4-methylpyridin-2-yl)amino]-1,2-dihydro-2,7-naphthyridin-1-one;
8-[(2,2-difluoroethyl)amino]-2-(2-hydroxyethyl)-6-(pyrazin-2-ylamino)-1,2-dihydro-2,7-naphthyridin-1-one;
8-(cyclopropylamino)-2-(2-hydroxyethyl)-6-[(4-methylpyridin-2-yl)amino]-1,2-dihydro-2,7-naphthyridin-1-one;
8-(cyclopropylamino)-2-(2-hydroxyethyl)-6-(pyrazin-2-ylamino)-1,2-dihydro-2,7-naphthyridin-1-one;
8-[(1-hydroxy-2-methylpropan-2-yl)amino]-6-[(4-methylpyridin-2-yl)amino]-2-(propan-2-yl)-1,2-dihydro-2,7-naphthyridin-1-one;
8-[(1-hydroxy-2-methylpropan-2-yl)amino]-2-(propan-2-yl)-6-(pyrazin-2-ylamino)-1,2-dihydro-2,7-naphthyridin-1-one;
8-(tert-butylamino)-6-[(4-methylpyridin-2-yl)amino]-2-(propan-2-yl)-1,2-dihydro-2,7-naphthyridin-1-one;
8-(tert-butylamino)-2-(propan-2-yl)-6-(pyrazin-2-ylamino)-1,2-dihydro-2,7-naphthyridin-1-one;
8-(tert-butylamino)-4-chloro-2-(2-hydroxyethyl)-6-(pyrazin-2-ylamino)-1,2-dihydro-2,7-naphthyridin-1-one;
8-(cyclopropylamino)-2-methyl-6-(pyrazin-2-ylamino)-1,2-dihydro-2,7-naphthyridin-1-one;
2-(2-hydroxyethyl)-6-(pyrazin-2-ylamino)-8-[(2,2,2-trifluoroethyl)amino]-1,2-dihydro-2,7-naphthyridin-1-one;
8-(tert-butylamino)-2-(2-hydroxyethyl)-6-[(4-hydroxypyrimidin-2-yl)amino]-1,2-dihydro-2,7-naphthyridin-1-one;
2-ethyl-8-{[(1S,3S)-3-(hydroxymethyl)cyclopentyl]amino}-6-(pyrazin-2-ylamino)-1,2-dihydro-2,7-naphthyridin-1-one;
8-(tert-butylamino)-2-(2-hydroxyethyl)-6-{[4-(pyridin-3-yl)pyridin-2-yl]amino}-1,2-dihydro-2,7-naphthyridin-1-one;
8-(tert-butylamino)-2-(2-hydroxyethyl)-6-[(4-phenylpyridin-2-yl)amino]-1,2-dihydro-2,7-naphthyridin-1-one;
8-(tert-butylamino)-2-(2-hydroxyethyl)-6-{[4-(1-methyl-1H-pyrazol-4-yl)pyridin-2-yl]amino}-1,2-dihydro-2,7-naphthyridin-1-one;
4-{[7-ethyl-8-oxo-3-(pyrazin-2-ylamino)-7,8-dihydro-2,7-naphthyridin-1-yl]amino}-N-(2-hydroxyethyl)benzamide;
2-ethyl-8-({4-[(4-hydroxypiperidin-1-yl)carbonyl]phenyl}amino)-6-(pyrazin-2-ylamino)-1,2-dihydro-2,7-naphthyridin-1-one;
2-ethyl-8-({4-[(3-hydroxypyrrolidin-1-yl)carbonyl]phenyl}amino)-6-(pyrazin-2-ylamino)-1,2-dihydro-2,7-naphthyridin-1-one;
4-({7-ethyl-3-[(4-methylpyridin-2-yl)amino]-8-oxo-7,8-dihydro-2,7-naphthyridin-1-yl}amino)-N-(2-hydroxyethyl)benzamide;
2-ethyl-8-({4-[(4-hydroxypiperidin-1-yl)carbonyl]phenyl}amino)-6-[(4-methylpyridin-2-yl)amino]-1,2-dihydro-2,7-naphthyridin-1-one;
2-ethyl-8-({4-[(3-hydroxypyrrolidin-1-yl)carbonyl]phenyl}amino)-6-[(4-methylpyridin-2-yl)amino]-1,2-dihydro-2,7-naphthyridin-1-one;
8-(tert-butylamino)-5-chloro-2-(2-hydroxyethyl)-6-(pyrazin-2-ylamino)-1,2-dihydro-2,7-naphthyridin-1-one;
2-(2-hydroxyethyl)-8-[(1-methylcyclopropyl)amino]-6-(pyrazin-2-ylamino)-1,2-dihydro-2,7-naphthyridin-1-one;
8-[(1-methylcyclopropyl)amino]-6-(pyrazin-2-ylamino)-1,2-dihydro-2,7-naphthyridin-1-one;
8-(tert-butylamino)-2-(2-hydroxyethyl)-6-{[4-(1H-pyrazol-4-yl)pyridin-2-yl]amino}-1,2-dihydro-2,7-naphthyridin-1-one;
8-(tert-butylamino)-2-(2-hydroxyethyl)-6-{[4-(pyridin-2-yl)pyridin-2-yl]amino}-1,2-dihydro-2,7-naphthyridin-1-one;
8-(tert-butylamino)-2-(2-hydroxyethyl)-6-{[4-(pyridin-4-yl)pyridin-2-yl]amino}-1,2-dihydro-2,7-naphthyridin-1-one;
8-(tert-butylamino)-2-(2-hydroxyethyl)-6-({4-[4-(hydroxymethyl)-1,3-thiazol-2-yl]pyridin-2-yl}amino)-1,2-dihydro-2,7-naphthyridin-1-one;
2-{[1-(tert-butylamino)-7-(2-hydroxyethyl)-8-oxo-7,8-dihydro-2,7-naphthyridin-3-yl]amino}pyridine-4-carbonitrile;
8-(tert-butylamino)-2-(2-hydroxyethyl)-6-({[4-(3-(hydroxymethyl)piperidin-1-yl]pyridin-2-yl}amino)-1,2-dihydro-2,7-naphthyridin-1-one;
8-(tert-butylamino)-2-(2-hydroxyethyl)-6-{[4-(4-hydroxypiperidin-1-yl)pyridin-2-yl]amino}-1,2-dihydro-2,7-naphthyridin-1-one;
8-(tert-butylamino)-2-(2-hydroxyethyl)-6-({4-[4-(hydroxymethyl)piperidin-1-yl]pyridin-2-yl}amino)-1,2-dihydro-2,7-naphthyridin-1-one;
8-(tert-butylamino)-2-(2-hydroxyethyl)-6-{[4-(3-hydroxypiperidin-1-yl)pyridin-2-yl]amino}-1,2-dihydro-2,7-naphthyridin-1-one;
2-[4-(2-{[1-(tert-butylamino)-7-(2-hydroxyethyl)-8-oxo-7,8-dihydro-2,7-naphthyridin-3-yl]amino}pyridin-4-yl)-1H-pyrazol-1-yl]acetamide;
8-(tert-butylamino)-2-(2-hydroxyethyl)-6-{[4-(1H-indazol-6-yl)pyridin-2-yl]amino}-1,2-dihydro-2,7-naphthyridin-1-one;
8-(tert-butylamino)-2-(2-hydroxyethyl)-6-{[4-(1H-indazol-5-yl)pyridin-2-yl]amino}-1,2-dihydro-2,7-naphthyridin-1-one;
2-(2-hydroxyethyl)-6-{[4-(4-hydroxypiperidin-1-yl)pyridin-2-yl]amino}-8-(propan-2-ylamino)-1,2-dihydro-2,7-naphthyridin-1-one;
8-(tert-butylamino)-2-(2-hydroxyethyl)-6-{[4-(3-methylpyridin-4-yl)pyridin-2-yl]amino}-1,2-dihydro-2,7-naphthyridin-1-one;
8-(tert-butylamino)-2-(2-hydroxyethyl)-6-{[4-(2-methylpyridin-4-yl)pyridin-2-yl]amino}-1,2-dihydro-2,7-naphthyridin-1-one;
8-(tert-butylamino)-2-(2-hydroxyethyl)-6-{[4-(5-methyl-1H-pyrazol-4-yl)pyridin-2-yl]amino}-1,2-dihydro-2,7-naphthyridin-1-one;
1-(2-{[1-(tert-butylamino)-7-(2-hydroxyethyl)-8-oxo-7,8-dihydro-2,7-naphthyridin-3-yl]amino}pyridin-4-yl)cyclopropane-1-carboxylic acid;
8-(tert-butylamino)-2-(2-hydroxyethyl)-6-{[4-(2-hydroxyethyl)pyridin-2-yl]amino}-1,2-dihydro-2,7-naphthyridin-1-one;
8-(tert-butylamino)-6-[(4-ethenylpyridin-2-yl)amino]-2-(2-hydroxyethyl)-1,2-dihydro-2,7-naphthyridin-1-one;
1-(2-{[1-(tert-butylamino)-7-(2-hydroxyethyl)-8-oxo-7,8-dihydro-2,7-naphthyridin-3-yl]amino}pyridin-4-yl)cyclopropane-1-carboxamide;
8-(tert-butylamino)-2-(2-hydroxyethyl)-6-({4-[1-(hydroxymethyl)cyclopropyl]pyridin-2-yl}amino)-1,2-dihydro-2,7-naphthyridin-1-one;
2-(2-{[1-(tert-butylamino)-7-(2-hydroxyethyl)-8-oxo-7,8-dihydro-2,7-naphthyridin-3-yl]amino}pyridin-4-yl)-2-methylpropanoic acid;
2-(2-{[1-(tert-butylamino)-7-(2-hydroxyethyl)-8-oxo-7,8-dihydro-2,7-naphthyridin-3-yl]amino}pyridin-4-yl)-2-methylpropanamide;
8-(tert-butylamino)-6-{[4-(1-hydroxy-2-methylpropan-2-yl)pyridin-2-yl]amino}-2-(2-hydroxyethyl)-1,2-dihydro-2,7-naphthyridin-1-one;
8-(tert-butylamino)-2-(2-hydroxyethyl)-6-{[4-(1-hydroxypropan-2-yl)pyridin-2-yl]amino}-1,2-dihydro-2,7-naphthyridin-1-one;
2-(2-{[1-(tert-butylamino)-7-(2-hydroxyethyl)-8-oxo-7,8-dihydro-2,7-naphthyridin-3-yl]amino}pyridin-4-yl)-2-methylpropanenitrile;
8-(tert-butylamino)-6-{[4-(2-hydroxyethoxy)pyridin-2-yl]amino}-2-(2-hydroxyethyl)-1,2-dihydro-2,7-naphthyridin-1-one;
8-(tert-butylamino)-6-{[5-fluoro-4-(1-hydroxyethyl)pyridin-2-yl]amino}-2-(2-hydroxyethyl)-1,2-dihydro-2,7-naphthyridin-1-one;
8-(tert-butylamino)-2-(2-hydroxyethyl)-6-{[4-(1-methoxyethyl)pyridin-2-yl]amino}-1,2-dihydro-2,7-naphthyridin-1-one;
2-{[1-(tert-butylamino)-7-(2-hydroxyethyl)-8-oxo-7,8-dihydro-2,7-naphthyridin-3-yl]amino}-N′-hydroxypyridine-4-carboximidamide;
8-(tert-butylamino)-6-{[4-(1-hydroxycyclobutyl)pyridin-2-yl]amino}-2-(2-hydroxyethyl)-1,2-dihydro-2,7-naphthyridin-1-one;
8-(tert-butylamino)-6-{[4-(1,1-difluoro-2-hydroxyethyl)pyridin-2-yl]amino}-2-(2-hydroxyethyl)-1,2-dihydro-2,7-naphthyridin-1-one;
8-(tert-butylamino)-6-{[4-(1-fluoro-2-hydroxyethyl)pyridin-2-yl]amino}-2-(2-hydroxyethyl)-1,2-dihydro-2,7-naphthyridin-1-one;
8-(tert-butylamino)-2-(2-hydroxyethyl)-6-{[4-(5-methyl-1,2,4-oxadiazol-3-yl)pyridin-2-yl]amino}-1,2-dihydro-2,7-naphthyridin-1-one;
8-(tert-butylamino)-6-{[4-(1,1-difluoroethyl)pyridin-2-yl]amino}-2-(2-hydroxyethyl)-1,2-dihydro-2,7-naphthyridin-1-one;
2-[8-(tert-butylamino)-6-{[4-(hydroxymethyl)pyridin-2-yl]amino}-1-oxo-1,2-dihydro-2,7-naphthyridin-2-yl]acetic acid;
8-(tert-butylamino)-6-{[5-fluoro-4-(1-hydroxyethyl)pyridin-2-yl]amino}-2-(2-hydroxyethyl)-1,2-dihydro-2,7-naphthyridin-1-one;
2-[8-(tert-butylamino)-6-{[5-fluoro-4-(2-hydroxypropan-2-yl)pyridin-2-yl]amino}-1-oxo-1,2-dihydro-2,7-naphthyridin-2-yl]acetamide;
8-(tert-butylamino)-2-(2-hydroxyethyl)-6-{[4-(pyrimidin-2-yl)pyridin-2-yl]amino}-1,2-dihydro-2,7-naphthyridin-1-one;
2-{[1-(tert-butylamino)-7-(2-hydroxyethyl)-8-oxo-7,8-dihydro-2,7-naphthyridin-3-yl]amino}-N-hydroxypyridine-4-carboxamide;
8-(tert-butylamino)-6-{[4-(2-hydroxy-2-methylpropyl)pyridin-2-yl]amino}-2-(2-hydroxyethyl)-1,2-dihydro-2,7-naphthyridin-1-one;
8-(tert-butylamino)-6-(pyrazin-2-ylamino)-2-(1H-1,2,3-triazol-5-ylmethyl)-1,2-dihydro-2,7-naphthyridin-1-one;
8-(tert-butylamino)-2-(prop-2-yn-1-yl)-6-(pyrazin-2-ylamino)-1,2-dihydro-2,7-naphthyridin-1-one;
8-(tert-butylamino)-2-(2-hydroxy-2-methylpropyl)-6-(pyrazin-2-ylamino)-1,2-dihydro-2,7-naphthyridin-1-one;
8-(tert-butylamino)-6-(pyrazin-2-ylamino)-2-(1H-1,2,3,4-tetrazol-5-ylmethyl)-1,2-dihydro-2,7-naphthyridin-1-one;
8-(tert-butylamino)-2-(2,3-dihydroxypropyl)-6-{[6-(2-methoxyethoxy)pyrimidin-4-yl]amino}-1,2-dihydro-2,7-naphthyridin-1-one;
8-(tert-butylamino)-2-(2-hydroxyethyl)-6-[(6-methylpyrimidin-4-yl)amino]-1,2-dihydro-2,7-naphthyridin-1-one;
8-(tert-butylamino)-2-(2,3-dihydroxypropyl)-6-{[6-(oxolan-3-ylmethoxy)pyrimidin-4-yl]amino}-1,2-dihydro-2,7-naphthyridin-1-one;
1-(6-{[1-(tert-butylamino)-7-(2-hydroxyethyl)-8-oxo-7,8-dihydro-2,7-naphthyridin-3-yl]amino}pyrimidin-4-yl)-1H-pyrazole-4-carboxylic acid;
ethyl 1-(6-{[1-(tert-butylamino)-7-(2-hydroxyethyl)-8-oxo-7,8-dihydro-2,7-naphthyridin-3-yl]amino}pyrimidin-4-yl)-1H-pyrazole-4-carboxylate;
8-(tert-butylamino)-2-[(2S)-2,3-dihydroxypropyl]-6-{[6-(1H-pyrazol-4-yloxy)pyrimidin-4-yl]amino}-1,2-dihydro-2,7-naphthyridin-1-one;
8-(tert-butylamino)-2-(2,3-dihydroxypropyl)-6-{[6-(oxolan-2-ylmethoxy)pyrimidin-4-yl]amino}-1,2-dihydro-2,7-naphthyridin-1-one;
6-(2-aminopyrimidin-5-yl)-2-(2-hydroxyethyl)-8-(propan-2-ylamino)-1,2-dihydro-2,7-naphthyridin-1-one;
6-(2-aminopyrimidin-5-yl)-8-[(2-methylbutan-2-yl)amino]-1,2-dihydro-2,7-naphthyridin-1-one;
6-(2-aminopyrimidin-5-yl)-8-(tert-butylamino)-1,2-dihydro-2,7-naphthyridin-1-one;
2-ethyl-8-[(1-hydroxy-2-methylpropan-2-yl)amino]-6-(pyrazin-2-ylamino)-1,2-dihydro-2,7-naphthyridin-1-one;
2-(2-hydroxyethyl)-8-[(1-methylcyclobutyl)amino]-6-(pyrazin-2-ylamino)-1,2-dihydro-2,7-naphthyridin-1-one;
2-ethyl-8-{[1-(hydroxymethyl)cyclobutyl]amino}-6-(pyrazin-2-ylamino)-1,2-dihydro-2,7-naphthyridin-1-one;
8-[(2,2-dimethylpropyl)amino]-2-(2-hydroxyethyl)-6-(pyrazin-2-ylamino)-1,2-dihydro-2,7-naphthyridin-1-one;
6-(2-aminopyrimidin-5-yl)-8-(tert-butylamino)-2-(2-hydroxyethyl)-1,2-dihydro-2,7-naphthyridin-1-one;
6-(6-aminopyridin-3-yl)-8-(tert-butylamino)-2-(2-hydroxyethyl)-1,2-dihydro-2,7-naphthyridin-1-one;
8-(tert-butylamino)-2-(2-hydroxyethyl)-6-{[4-(trifluoromethyl)pyridin-2-yl]amino}-1,2-dihydro-2,7-naphthyridin-1-one;
6-(2-aminopyrimidin-5-yl)-8-[(1-hydroxy-2-methylpropan-2-yl)amino]-2-(2-hydroxyethyl)-1,2-dihydro-2,7-naphthyridin-1-one;
6-(2-aminopyrimidin-5-yl)-2-(2-hydroxyethyl)-8-{[1-(hydroxymethyl)cyclobutyl]amino}-1,2-dihydro-2,7-naphthyridin-1-one;
6-(2-aminopyrimidin-5-yl)-2-ethyl-8-[(1-hydroxy-2-methylpropan-2-yl)amino]-1,2-dihydro-2,7-naphthyridin-1-one;
6-(2-aminopyrimidin-5-yl)-2-ethyl-8-{[(1-(hydroxymethyl)cyclobutyl]amino}-1,2-dihydro-2,7-naphthyridin-1-one;
6-(2-aminopyrimidin-5-yl)-2-ethyl-8-[(1-methylcyclobutyl)amino]-1,2-dihydro-2,7-naphthyridin-1-one;
6-(2-aminopyrimidin-5-yl)-2-(2-hydroxyethyl)-8-[(1-methylcyclobutyl)amino]-1,2-dihydro-2,7-naphthyridin-1-one;
6-(2-aminopyrimidin-5-yl)-2-(2-hydroxyethyl)-8-[(2-methylbutan-2-yl)amino]-1,2-dihydro-2,7-naphthyridin-1-one;
6-(2-aminopyrimidin-5-yl)-2-(2-hydroxyethyl)-8-(pyrrolidin-1-yl)-1,2-dihydro-2,7-naphthyridin-1-one;
6-(2-aminopyrimidin-5-yl)-8-(tert-butylamino)-2-(2-methoxyethyl)-1,2-dihydro-2,7-naphthyridin-1-one;
6-(2-aminopyrimidin-5-yl)-8-[(2,2-dimethylpropyl)amino]-2-(2-hydroxyethyl)-1,2-dihydro-2,7-naphthyridin-1-one;
6-(2-aminopyrimidin-5-yl)-2-(2-hydroxyethyl)-8-{[(1S,3R)-3-(hydroxymethyl)cyclopentyl]amino}-1,2-dihydro-2,7-naphthyridin-1-one;
6-(2-aminopyrimidin-5-yl)-2-ethyl-8-{[(1S,3R)-3-(hydroxymethyl)cyclopentyl]amino}-1,2-dihydro-2,7-naphthyridin-1-one;
6-(2-aminopyrimidin-5-yl)-8-(tert-butylamino)-2-propyl-1,2-dihydro-2,7-naphthyridin-1-one;
6-(2-aminopyrimidin-5-yl)-8-(tert-butylamino)-2-(3-hydroxypropyl)-1,2-dihydro-2,7-naphthyridin-1-one;
8-(tert-butylamino)-2-(2-hydroxyethyl)-6-[2-(methylamino)pyrimidin-5-yl]-1,2-dihydro-2,7-naphthyridin-1-one;
2-(2-hydroxyethyl)-8-[(2-methylbutan-2-yl)amino]-6-(pyrazin-2-ylamino)-1,2-dihydro-2,7-naphthyridin-1-one;
6-(2-aminopyrimidin-5-yl)-8-(tert-butylamino)-2-(oxolan-2-ylmethyl)-1,2-dihydro-2,7-naphthyridin-1-one;
6-(2-aminopyrimidin-5-yl)-8-(tert-butylamino)-2-(oxan-4-ylmethyl)-1,2-dihydro-2,7-naphthyridin-1-one;
6-(2-aminopyrimidin-5-yl)-8-(tert-butylamino)-2-(4-hydroxybutyl)-1,2-dihydro-2,7-naphthyridin-1-one;
6-(2-aminopyrimidin-5-yl)-8-(tert-butylamino)-2-(oxetan-3-yl)-1,2-dihydro-2,7-naphthyridin-1-one;
6-(2-aminopyrimidin-5-yl)-8-(tert-butylamino)-2-(1,3-dihydroxypropan-2-yl)-1,2-dihydro-2,7-naphthyridin-1-one;
6-(2-aminopyrimidin-5-yl)-8-(tert-butylamino)-2-{[3-(hydroxymethyl)oxetan-3-yl]methyl}-1,2-dihydro-2,7-naphthyridin-1-one;
6-(2-aminopyrimidin-5-yl)-8-(tert-butylamino)-2-[(3-methyloxetan-3-yl)methyl]-1,2-dihydro-2,7-naphthyridin-1-one;
6-(2-aminopyrimidin-5-yl)-8-{[2-(4-fluorophenyl)propan-2-yl]amino}-2-(2-hydroxyethyl)-1,2-dihydro-2,7-naphthyridin-1-one;
6-(2-aminopyrimidin-5-yl)-8-(tert-butylamino)-2-(3,3,3-trifluoro-2-hydroxypropyl)-1,2-dihydro-2,7-naphthyridin-1-one;
6-(2-aminopyrimidin-5-yl)-8-(tert-butylamino)-2-(2-hydroxypropyl)-1,2-dihydro-2,7-naphthyridin-1-one;
6-(2-aminopyrimidin-5-yl)-8-(tert-butylamino)-2-(2-hydroxy-2-methylpropyl)-1,2-dihydro-2,7-naphthyridin-1-one;
6-(2-aminopyrimidin-5-yl)-8-(tert-butylamino)-2-(2,3-dihydroxypropyl)-1,2-dihydro-2,7-naphthyridin-1-one;
6-(2-aminopyrimidin-5-yl)-8-[(1-methylcyclobutyl)amino]-2-(oxan-4-ylmethyl)-1,2-dihydro-2,7-naphthyridin-1-one;
6-(2-aminopyrimidin-5-yl)-8-[(1-methylcyclobutyl)amino]-2-(oxetan-3-yl)-1,2-dihydro-2,7-naphthyridin-1-one;
6-(2-aminopyrimidin-5-yl)-8-[(1-methylcyclobutyl)amino]-2-[(3-methyloxetan-3-yl)methyl]-1,2-dihydro-2,7-naphthyridin-1-one;
6-(2-aminopyrimidin-5-yl)-2-{[3-(hydroxymethyl)oxetan-3-yl]methyl}-8-[(1-methylcyclobutyl)amino]-1,2-dihydro-2,7-naphthyridin-1-one;
6-(2-aminopyrimidin-5-yl)-8-(tert-butylamino)-2-[(2S)-2,3-dihydroxypropyl]-1,2-dihydro-2,7-naphthyridin-1-one;
6-(2-aminopyrimidin-5-yl)-8-(tert-butylamino)-2-[(2R)-2,3-dihydroxypropyl]-1,2-dihydro-2,7-naphthyridin-1-one;
6-(2-aminopyrimidin-5-yl)-8-(tert-butylamino)-2-(1,3-dimethoxypropan-2-yl)-1,2-dihydro-2,7-naphthyridin-1-one;
8-(tert-butylamino)-2-(1,3-dimethoxypropan-2-yl)-6-(pyrazin-2-ylamino)-1,2-dihydro-2,7-naphthyridin-1-one;
2-ethyl-8-{[(1S,2R)-2-hydroxycyclopentyl]amino}-6-(pyrazin-2-ylamino)-1,2-dihydro-2,7-naphthyridin-1-one;
2-ethyl-8-{[(1R,2R)-2-hydroxycyclopentyl]amino}-6-[(4-methylpyridin-2-yl)amino]-1,2-dihydro-2,7-naphthyridin-1-one;
2-ethyl-8-{[(1R,2R)-2-hydroxycyclopentyl]amino}-6-(pyrazin-2-ylamino)-1,2-dihydro-2,7-naphthyridin-1-one;
2-ethyl-8-{[(1R,2S)-2-(hydroxymethyl)cyclopentyl]amino}-6-[(4-methylpyridin-2-yl)amino]-1,2-dihydro-2,7-naphthyridin-1-one;
2-ethyl-8-{[(1R,2S)-2-(hydroxymethyl)cyclopentyl]amino}-6-(pyrazin-2-ylamino)-1,2-dihydro-2,7-naphthyridin-1-one;
8-[(4-hydroxycyclohexyl)amino]-6-[(4-methylpyridin-2-yl)amino]-2-(propan-2-yl)-1,2-dihydro-2,7-naphthyridin-1-one;
8-[(4-hydroxycyclohexyl)amino]-2-(propan-2-yl)-6-(pyrazin-2-ylamino)-1,2-dihydro-2,7-naphthyridin-1-one;
2-ethyl-8-{[(1S,3S)-3-hydroxycyclopentyl]amino}-6-[(4-methylpyridin-2-yl)amino]-1,2-dihydro-2,7-naphthyridin-1-one;
2-ethyl-8-{[(1S,3S)-3-hydroxycyclopentyl]amino}-6-(pyrazin-2-ylamino)-1,2-dihydro-2,7-naphthyridin-1-one;
8-[(4-hydroxycyclohexyl)amino]-2-(3-hydroxypropyl)-6-[(4-methylpyridin-2-yl)amino]-1,2-dihydro-2,7-naphthyridin-1-one;
8-[(4-hydroxycyclohexyl)amino]-2-(3-hydroxypropyl)-6-(pyrazin-2-ylamino)-1,2-dihydro-2,7-naphthyridin-1-one;
2-ethyl-8-[(4-hydroxycyclohexyl)amino]-6-(pyrazin-2-ylamino)-1,2-dihydro-2,7-naphthyridin-1-one;
2-ethyl-8-[(4-hydroxy-4-methylcyclohexyl)amino]-6-[(4-methylpyridin-2-yl)amino]-1,2-dihydro-2,7-naphthyridin-1-one;
2-ethyl-8-[(4-hydroxy-4-methylcyclohexyl)amino]-6-[(4-methylpyridin-2-yl)amino]-1,2-dihydro-2,7-naphthyridin-1-one;
2-ethyl-8-[(4-hydroxy-4-methylcyclohexyl)amino]-6-(pyrazin-2-ylamino)-1,2-dihydro-2,7-naphthyridin-1-one;
2-ethyl-8-[(4-hydroxy-4-methylcyclohexyl)amino]-6-(pyrazin-2-ylamino)-1,2-dihydro-2,7-naphthyridin-1-one;
2-ethyl-8-[(4-methylcyclohex-3-en-1-yl)amino]-6-[(4-methylpyridin-2-yl)amino]-1,2-dihydro-2,7-naphthyridin-1-one;
2-ethyl-8-[(4-methylcyclohex-3-en-1-yl)amino]-6-(pyrazin-2-ylamino)-1,2-dihydro-2,7-naphthyridin-1-one;
2-ethyl-8-{[(1S,2S)-2-hydroxycyclopentyl]amino}-6-[(4-hydroxypyrimidin-2-yl)amino]-1,2-dihydro-2,7-naphthyridin-1-one;
2-ethyl-8-{[(1S,2R)-2-hydroxycyclopentyl]amino}-6-[(4-hydroxypyrimidin-2-yl)amino]-1,2-dihydro-2,7-naphthyridin-1-one;
2-ethyl-8-{[(1R,3S)-3-hydroxycyclopentyl]amino}-6-[(4-hydroxypyrimidin-2-yl)amino]-1,2-dihydro-2,7-naphthyridin-1-one;
2-ethyl-8-({[(1R,3R)-3-hydroxycyclopentyl]methyl}amino)-6-[(4-hydroxypyrimidin-2-yl)amino]-1,2-dihydro-2,7-naphthyridin-1-one;
2-ethyl-8-{[(1R,3R)-3-hydroxycyclopentyl]amino}-6-[(4-hydroxypyrimidin-2-yl)amino]-1,2-dihydro-2,7-naphthyridin-1-one;
2-ethyl-8-{[(1S,3R)-3-hydroxycyclopentyl]amino}-6-[(4-hydroxypyrimidin-2-yl)amino]-1,2-dihydro-2,7-naphthyridin-1-one;
2-ethyl-8-{[(2R)-1-hydroxypropan-2-yl]amino}-6-[(4-methylpyridin-2-yl)amino]-1,2-dihydro-2,7-naphthyridin-1-one;
2-ethyl-8-{[(2R)-1-hydroxypropan-2-yl]amino}-6-(pyrazin-2-ylamino)-1,2-dihydro-2,7-naphthyridin-1-one;
8-[(4-hydroxycyclohexyl)amino]-6-[(4-methylpyridin-2-yl)amino]-2-(propan-2-yl)-1,2-dihydro-2,7-naphthyridin-1-one;
8-[(4-hydroxycyclohexyl)amino]-2-(propan-2-yl)-6-(pyrazin-2-ylamino)-1,2-dihydro-2,7-naphthyridin-1-one;
8-{[(2S)-1-hydroxypropan-2-yl]amino}-6-[(4-methylpyridin-2-yl)amino]-2-(propan-2-yl)-1,2-dihydro-2,7-naphthyridin-1-one;
8-{[(2S)-1-hydroxypropan-2-yl]amino}-2-(propan-2-yl)-6-(pyrazin-2-ylamino)-1,2-dihydro-2,7-naphthyridin-1-one;
2-ethyl-6-[(4-methylpyridin-2-yl)amino]-8-{[(3R)-6-oxopiperidin-3-yl]amino}-1,2-dihydro-2,7-naphthyridin-1-one;
2-ethyl-8-{[(3R)-6-oxopiperidin-3-yl]amino}-6-(pyrazin-2-ylamino)-1,2-dihydro-2,7-naphthyridin-1-one;
3-({7-ethyl-3-[(4-methylpyridin-2-yl)amino]-8-oxo-7,8-dihydro-2,7-naphthyridin-1-yl}amino)-1$1̂{6}-thiolane-1,1-dione;
3-{[7-ethyl-8-oxo-3-(pyrazin-2-ylamino)-7,8-dihydro-2,7-naphthyridin-1-yl]amino}-1$1̂{6}-thiolane-1,1-dione;
2-ethyl-8-[(3-hydroxycyclohexyl)amino]-6-[(4-methylpyridin-2-yl)amino]-1,2-dihydro-2,7-naphthyridin-1-one;
2-ethyl-8-[(3-hydroxycyclohexyl)amino]-6-(pyrazin-2-ylamino)-1,2-dihydro-2,7-naphthyridin-1-one;
3-{[7-(2-hydroxyethyl)-3-[(4-methylpyridin-2-yl)amino]-8-oxo-7,8-dihydro-2,7-naphthyridin-1-yl]amino}-1$1̂{6}-thiolane-1,1-dione;
3-{[7-(2-hydroxyethyl)-8-oxo-3-(pyrazin-2-ylamino)-7,8-dihydro-2,7-naphthyridin-1-yl]amino}-1$1̂{6}-thiolane-1,1-dione;
8-[(4-hydroxycyclohexyl)amino]-6-(pyrazin-2-ylamino)-1,2-dihydro-2,7-naphthyridin-1-one;
8-{[(1S,3R)-3-hydroxycyclopentyl]amino}-6-[(4-methylpyridin-2-yl)amino]-2-(propan-2-yl)-1,2-dihydro-2,7-naphthyridin-1-one;
8-{[(1S,3R)-3-hydroxycyclopentyl]amino}-2-(propan-2-yl)-6-(pyrazin-2-ylamino)-1,2-dihydro-2,7-naphthyridin-1-one;
8-{[(1S,2R)-2-hydroxycyclopentyl]amino}-6-[(4-methylpyridin-2-yl)amino]-2-(propan-2-yl)-1,2-dihydro-2,7-naphthyridin-1-one;
8-{[(1S,2R)-2-hydroxycyclopentyl]amino}-2-(propan-2-yl)-6-(pyrazin-2-ylamino)-1,2-dihydro-2,7-naphthyridin-1-one;
8-[(4-hydroxycyclohexyl)amino]-6-(pyrazin-2-ylamino)-1,2-dihydro-2,7-naphthyridin-1-one;
8-[(4-hydroxycyclohexyl)amino]-2-(2-methanesulfonylethyl)-6-(pyrazin-2-ylamino)-1,2-dihydro-2,7-naphthyridin-1-one;
8-[(4-hydroxycyclohexyl)amino]-2-methyl-6-[(4-methylpyridin-2-yl)amino]-1,2-dihydro-2,7-naphthyridin-1-one;
8-[(4-hydroxycyclohexyl)amino]-2-methyl-6-(pyrazin-2-ylamino)-1,2-dihydro-2,7-naphthyridin-1-one;
8-{[(1S,3R)-3-(hydroxymethyl)cyclopentyl]amino}-2-methyl-6-[(4-methylpyridin-2-yl)amino]-1,2-dihydro-2,7-naphthyridin-1-one;
8-{[(1S,3R)-3-(hydroxymethyl)cyclopentyl]amino}-2-methyl-6-(pyrazin-2-ylamino)-1,2-dihydro-2,7-naphthyridin-1-one;
8-{[(1S,3R)-3-(hydroxymethyl)cyclopentyl]amino}-6-[(4-methylpyridin-2-yl)amino]-2-(propan-2-yl)-1,2-dihydro-2,7-naphthyridin-1-one;
8-{[(1S,3R)-3-(hydroxymethyl)cyclopentyl]amino}-2-(propan-2-yl)-6-(pyrazin-2-ylamino)-1,2-dihydro-2,7-naphthyridin-1-one;
8-[(4-hydroxycyclohexyl)amino]-2-(2-hydroxyethyl)-6-(pyrazin-2-ylamino)-1,2-dihydro-2,7-naphthyridin-1-one;
2-ethyl-8-[(4-hydrogenio-4-hydroxycyclohexyl)amino]-6-[(4-methylpyridin-2-yl)amino]-1,2-dihydro-2,7-naphthyridin-1-one;
2-ethyl-8-[(4-hydrogenio-4-hydroxycyclohexyl)amino]-6-(pyrazin-2-ylamino)-1,2-dihydro-2,7-naphthyridin-1-one;
2-ethyl-8-[(4-hydrogenio-4-hydroxycyclohexyl)amino]-6-(pyrazin-2-ylamino)-1,2-dihydro-2,7-naphthyridin-1-one;
8-[(4-hydroxycyclohexyl)amino]-6-[(4-methylpyridin-2-yl)amino]-2-[2-(morpholin-4-yl)ethyl]-1,2-dihydro-2,7-naphthyridin-1-one;
8-[(4-hydroxycyclohexyl)amino]-2-[2-(morpholin-4-yl)ethyl]-6-(pyrazin-2-ylamino)-1,2-dihydro-2,7-naphthyridin-1-one;
8-{[(2S)-1-hydroxypropan-2-yl]amino}-6-[(4-methylpyridin-2-yl)amino]-2-[2-(morpholin-4-yl)ethyl]-1,2-dihydro-2,7-naphthyridin-1-one;
8-{[(2S)-1-hydroxypropan-2-yl]amino}-2-[2-(morpholin-4-yl)ethyl]-6-(pyrazin-2-ylamino)-1,2-dihydro-2,7-naphthyridin-1-one;
8-(tert-butylamino)-6-[(4-methylpyridin-2-yl)amino]-2-[2-(morpholin-4-yl)ethyl]-1,2-dihydro-2,7-naphthyridin-1-one;
8-(tert-butylamino)-2-[2-(morpholin-4-yl)ethyl]-6-(pyrazin-2-ylamino)-1,2-dihydro-2,7-naphthyridin-1-one;
2-(2-fluoroethyl)-8-[(4-hydroxycyclohexyl)amino]-6-(pyrazin-2-ylamino)-1,2-dihydro-2,7-naphthyridin-1-one;
2-(2-fluoroethyl)-8-{[(2S)-1-hydroxypropan-2-yl]amino}-6-(pyrazin-2-ylamino)-1,2-dihydro-2,7-naphthyridin-1-one;
8-(tert-butylamino)-2-(2-fluoroethyl)-6-(pyrazin-2-ylamino)-1,2-dihydro-2,7-naphthyridin-1-one;
3-{8-[(4-hydroxycyclohexyl)amino]-1-oxo-6-(pyrazin-2-ylamino)-1,2-dihydro-2,7-naphthyridin-2-yl}propanenitrile;
3-(8-{[(2S)-1-hydroxypropan-2-yl]amino}-1-oxo-6-(pyrazin-2-ylamino)-1,2-dihydro-2,7-naphthyridin-2-yl)propanenitrile;
3-[8-(tert-butylamino)-1-oxo-6-(pyrazin-2-ylamino)-1,2-dihydro-2,7-naphthyridin-2-yl]propanenitrile;
8-[(4-hydroxycyclohexyl)amino]-2-(2-methoxyethyl)-6-(pyrazin-2-ylamino)-1,2-dihydro-2,7-naphthyridin-1-one;
8-(tert-butylamino)-2-(2-methoxyethyl)-6-(pyrazin-2-ylamino)-1,2-dihydro-2,7-naphthyridin-1-one;
8-[(4-hydroxycyclohexyl)amino]-2-[2-(morpholin-4-yl)-2-oxoethyl]-6-(pyrazin-2-ylamino)-1,2-dihydro-2,7-naphthyridin-1-one;
8-(tert-butylamino)-2-[2-(morpholin-4-yl)-2-oxoethyl]-6-(pyrazin-2-ylamino)-1,2-dihydro-2,7-naphthyridin-1-one;
8-[(4-hydroxycyclohexyl)amino]-2-(oxetan-3-yl)-6-(pyrazin-2-ylamino)-1,2-dihydro-2,7-naphthyridin-1-one;
3-{8-[(4-hydroxycyclohexyl)amino]-1-oxo-6-(pyrazin-2-ylamino)-1,2-dihydro-2,7-naphthyridin-2-yl}-1$1̂{6}-thietane-1,1-dione;
8-(tert-butylamino)-6-(pyrazin-2-ylamino)-2-(3,3,3-trifluoro-2-hydroxypropyl)-1,2-dihydro-2,7-naphthyridin-1-one;
8-[(4-hydroxycyclohexyl)amino]-6-(pyrazin-2-ylamino)-2-(3,3,3-trifluoro-2-hydroxypropyl)-1,2-dihydro-2,7-naphthyridin-1-one;
8-(tert-butylamino)-2-[2-(2-hydroxyethoxy)ethyl]-6-(pyrazin-2-ylamino)-1,2-dihydro-2,7-naphthyridin-1-one;
8-(tert-butylamino)-2-(oxetan-3-yl)-6-(pyrazin-2-ylamino)-1,2-dihydro-2,7-naphthyridin-1-one;
3-[8-(tert-butylamino)-1-oxo-6-(pyrazin-2-ylamino)-1,2-dihydro-2,7-naphthyridin-2-yl]-1$1̂{6}-thietane-1,1-dione;
3-({1-[(4-hydroxycyclohexyl)amino]-8-oxo-7-(propan-2-yl)-7,8-dihydro-2,7-naphthyridin-3-yl}amino)pyrazin-1-ium-1-olate;
3-{[(1-[(1S,3R)-3-hydroxycyclopentyl]amino}-8-oxo-7-(propan-2-yl)-7,8-dihydro-2,7-naphthyridin-3-yl)amino]pyrazin-1-ium-1-olate;
3-[(1-{[(1S,3R)-3-(hydroxymethyl)cyclopentyl]amino}-8-oxo-7-(propan-2-yl)-7,8-dihydro-2,7-naphthyridin-3-yl)amino]pyrazin-1-ium-1-olate;
8-(tert-butylamino)-6-(pyrazin-2-ylamino)-2-(pyridin-4-ylmethyl)-1,2-dihydro-2,7-naphthyridin-1-one;
8-(tert-butylamino)-6-(pyrazin-2-ylamino)-2-(pyridin-3-ylmethyl)-1,2-dihydro-2,7-naphthyridin-1-one;
8-(tert-butylamino)-6-(pyrazin-2-ylamino)-2-(pyridin-2-ylmethyl)-1,2-dihydro-2,7-naphthyridin-1-one;
2-(2,2-difluoroethyl)-8-[(4-hydroxycyclohexyl)amino]-6-(pyrazin-2-ylamino)-1,2-dihydro-2,7-naphthyridin-1-one;
8-(tert-butylamino)-2-(2,2-difluoroethyl)-6-(pyrazin-2-ylamino)-1,2-dihydro-2,7-naphthyridin-1-one;
8-[(4-hydroxycyclohexyl)amino]-6-(pyrazin-2-ylamino)-2-(pyridin-4-ylmethyl)-1,2-dihydro-2,7-naphthyridin-1-one;
8-[(4-hydroxycyclohexyl)amino]-6-(pyrazin-2-ylamino)-2-(2,2,2-trifluoroethyl)-1,2-dihydro-2,7-naphthyridin-1-one;
6-(2-aminopyrimidin-5-yl)-8-[(4-hydroxycyclohexyl)amino]-2-methyl-1,2-dihydro-2,7-naphthyridin-1-one;
6-(2-aminopyrimidin-5-yl)-8-[(4-hydroxycyclohexyl)amino]-2-(propan-2-yl)-1,2-dihydro-2,7-naphthyridin-1-one;
6-(2-aminopyrimidin-5-yl)-8-[(4-hydroxycyclohexyl)amino]-2-(2-hydroxyethyl)-1,2-dihydro-2,7-naphthyridin-1-one;
6-(2-aminopyrimidin-5-yl)-8-[(4-hydroxycyclohexyl)amino]-2-[2-(morpholin-4-yl)ethyl]-1,2-dihydro-2,7-naphthyridin-1-one;
6-(2-aminopyrimidin-5-yl)-8-{[(1S,3R)-3-(hydroxymethyl)cyclopentyl]amino}-2-(propan-2-yl)-1,2-dihydro-2,7-naphthyridin-1-one;
6-(2-aminopyrimidin-5-yl)-8-{[(1S,3R)-3-hydroxycyclopentyl]amino}-2-(propan-2-yl)-1,2-dihydro-2,7-naphthyridin-1-one;
6-(2-aminopyrimidin-5-yl)-2-ethyl-8-[(4-hydroxycyclohexyl)amino]-1,2-dihydro-2,7-naphthyridin-1-one;
6-(2-aminopyrimidin-5-yl)-8-(tert-butylamino)-2-[2-(morpholin-4-yl)ethyl]-1,2-dihydro-2,7-naphthyridin-1-one;
8-(tert-butylamino)-2-(3-hydroxypropyl)-6-(pyrazin-2-ylamino)-1,2-dihydro-2,7-naphthyridin-1-one;
6-(2-aminopyrimidin-5-yl)-8-(cyclopropylamino)-2-(2-hydroxyethyl)-1,2-dihydro-2,7-naphthyridin-1-one;
6-(2-aminopyrimidin-5-yl)-2-(2-hydroxyethyl)-8-[(1-methylcyclopropyl)amino]-1,2-dihydro-2,7-naphthyridin-1-one;
6-(2-aminopyrimidin-5-yl)-2-ethyl-8-[(4-hydroxy-2-methylbutan-2-yl)amino]-1,2-dihydro-2,7-naphthyridin-1-one;
6-(2-aminopyrimidin-5-yl)-2-ethyl-8-{[1-(hydroxymethyl)cyclopropyl]amino}-1,2-dihydro-2,7-naphthyridin-1-one, and
6-(2-aminopyrimidin-5-yl)-2-ethyl-8-{[(1S,3R)-3-hydroxycyclopentyl]amino}-1,2-dihydro-2,7-naphthyridin-1-one.

29. A pharmaceutical composition for treating a Syk kinase mediated disease comprising a therapeutically effective amount of a compound of Formula (I) of claim 1 and a pharmaceutically acceptable excipient.

30. A medicament for treating a Syk kinase mediated disease, wherein the medicament comprises a therapeutically effective amount of a compound of Formula (I) of claim 1.

31. Use of a compound of a compound of Formula (I) of claim 1 in the manufacture of a medicament for treating a Syk-mediated disease in a subject in need thereof.

32. A method for inhibiting a Syk kinase, comprising administering to a system or a subject in need thereof a therapeutically effective amount of a compound of Formula (I) of claim 1.

33. A method for treating a Syk-mediated disease comprising administering to a subject in need thereof a therapeutically effective amount of a compound of Formula (I) of claim 1.

34. The method of claim 33, wherein the disease is an inflammatory disease, an allergic disease, a cell-proliferative disease, an autoimmune disease or cytopenia.

35. The method of claim 33, wherein the disease is allergic asthma, allergic rhinitis, rheumatoid arthritis, multiple sclerosis, lupus, systemic lupus erythematosus, lymphoma, B cell lymphoma, T cell lymphoma, myelodysplasic syndrome, anemia, leucopenia, neutropenia, thrombocytopenia, granuloctopenia, pancytoia or idiopathic thrombocytopenic purpura.

36. A compound for use in a method of medical treatment, wherein the method of medical treatment is for treating a Syk kinase mediated disease, wherein the disease is selected from allergic asthma, allergic rhinitis, rheumatoid arthritis, multiple sclerosis, lupus, systemic lupus erythematosus, lymphoma, B cell lymphoma, T cell lymphoma, myelodysplasic syndrome, anemia, leucopenia, neutropenia, thrombocytopenia, granuloctopenia, pancytoia and idiopathic thrombocytopenic purpura, and wherein the compound is a compound of Formula (I) of claim 1.
